(19) United States
(12) Patent Application Publication
(10) Pub. No.: US 2023/0234917 A1
(43) Pub. Date: Jul. 27, 2023

(54) ASPARAGINE DERIVATIVES AND METHODS OF USING SAME

(71) Applicant: SENDA BIOSCIENCES, INC., Cambridge, MA (US)

(72) Inventors: Spencer Cory PECK, Watertown, MA (US); Jenny LIU, Cambridge, MA (US); Timothy F. BRIGGS, Waltham, MA (US); John Robert PROUDFOOT, Newton, CT (US); William MCELROY, Cambridge, MA (US); Robert Walter MYERS, Cresskill, NJ (US); Steven John TAYLOR, Winchester, MA (US)

(21) Appl. No.: 18/001,238

(22) PCT Filed: Jun. 9, 2021
(86) PCT No.: PCT/US2021/036634

§ 371 Date: Dec. 8, 2022

Related U.S. Application Data
(60) Provisional application No. 63/036,961, filed on Jun. 9, 2020.
(60) Provisional application No. 63/112,784, filed on Nov. 12, 2020.

(51) Int. Cl. C07C 237/10; A61P 35/00; C07K 5/062; C07D 305/08; C07C 275/14; C07C 271/20; C07F 9/40; C07F 9/42; C07C 317/08; C07F 5/02; C07C 237/22; C07D 263/32; C07D 233/64; C07D 257/04; C07D 249/08; C07D 271/06; C07D 271/10; C07D 249/04; C07C 255/29; C07D 213/40; C07D 333/20; C07D 235/16

(52) U.S. Cl. CPC C07C 237/10; A61P 35/00; C07K 5/06026; C07D 305/08; C07C 275/14; C07C 271/20; C07F 9/4006; C07F 9/425; C07C 317/08; C07F 5/027; C07C 237/22; C07D 263/32; C07D 233/64; C07D 257/04; C07D 249/08; C07D 271/06; C07D 271/10; C07D 249/04; C07C 255/29; C07D 213/40; C07D 333/20; C07D 235/16; C07C 2601/14

(57) ABSTRACT

The present disclosure relates to compounds of formulas (A) and (I), pharmaceutically acceptable salts thereof, and solvates of any of the foregoing, pharmaceutical compositions comprising the same, methods of preparing the same, intermediate compounds useful for preparing the same, and methods for treating or prophylaxis of diseases, in particular cancer, such as colorectal cancer, using the same.

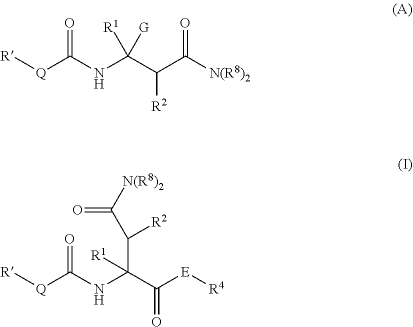

This application claims the benefit of priority to U.S. Provisional Patent Application No. 63/036,961, filed Jun. 9, 2020, and U.S. Provisional Patent Application No. 63/112,784, filed Nov. 12, 2020, which are incorporated herein by reference in their entirety.

Disclosed herein are novel compounds of formula (A) and pharmaceutically acceptable salts thereof, pharmaceutical compositions comprising the same, methods of preparing the same, intermediate compounds useful for preparing the same, and methods for treating or prophylaxis of diseases, in particular cancer, such as colorectal cancer, using the same.

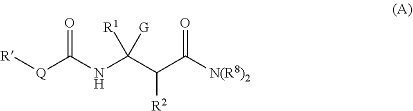

Colorectal cancer (CRC) is the third most frequent cancer in the world, with an incidence of 1.2 million new cases and more than 600,000 deaths a year. Inherited forms of the disease represent only 5 to 10% of cases while 1 to 2% occur in a context of inflammatory bowel diseases. Almost all cases (about 90%) of CRC are therefore sporadic and influenced by environmental factors such as diet or intestinal microbiota. Overall CRC survival rate at 5 years at any stage of the disease is 50%, but only 10% in metastatic stages. It is therefore essential to prevent, detect and treat this cancer efficiently before the occurrence of metastasis. Resecting surgery is usually the primary or first treatment. Chemotherapy and radiotherapy can also be proposed to patients, as an alternative to surgery if the latter is impossible, or in addition to surgery (which is frequently the case for stage III patients and stage II patients with high recurrence risk). In stage IV patients (when the cancer has spread to distant organs/tissues), surgery is unable to cure the disease and most patients receive chemotherapy.

Chemotherapy is thus an important part of CRC treatment and numerous chemotherapy regimens are currently available for patients. Recently, the therapeutic arsenal available for CRC has doubled with the emergence of targeted therapies. Indeed, in addition to the chemotherapy molecules classically used (5-fluorouracil, capecitabin, irinotecan and oxaliplatin), targeted therapies have recently been FDA approved (bevacizumab, aflibercept, cetuximab, panitumumab, and regorafenib). These new treatments target specific biological functions or alterations found in cancer cells or pathways that are crucial for cancer development such as vascular endothelial growth factor (VEGF), a pro-angiogenic pathway, and epidermal growth factor receptor (EGFR). The combined use of these molecules with additional cytotoxic molecules has improved patient survival. However, it has been observed that the efficacy of the treatment depends on the type of mutations found in the tumor. For example, chemotherapy adjuvant treatment was not beneficial in patients with microsatellite instability/mismatch repair (MSI/MMR) and is therefore not recommended in the MSI/MMR CRCs. Anti-EGFR molecules should only be used in patients with RAS wild-type tumors. Thus, investigating ras mutation status has become an important aspect of diagnosis and pre-therapeutic evaluation and is an indispensable prerequisite in choosing the most suitable treatment.

Although genetics plays an important role in cancers, increasing evidence supports the part played by infections in the occurrence of cancers. They can induce tumorigenesis and/or sustain tumor growth and progression. Gut microbiota is highly suspected to play an important role in CRC and therefore has become a wide area of investigation in recent years. Studies comparing bacteria associated with tumor tissue and with tumor-adjacent mucosa of CRC patients, versus bacteria associated with the mucosa of healthy patients have demonstrated the existence of a dysbiotic microbiota associated with CRC. Although it is still difficult to identify a “typical” dysbiotic microbiota associated with CRC, an abnormal abundance of certain species in colonic tissues of CRC patients has been found in numerous studies. Thus, studies of CRC have observed a specific enrichment in Fusobacterium and Bacteroides bacteria that may potentially play a role in colorectal carcinogenesis. In contrast, microbiota from healthy patients seems to be enriched in Bifidobacterium, which are generally anti-inflammatory bacteria. Interestingly, early signs of dysbiosis have been reported at the adenoma stage, and there is a significant increase in some species (such as Bacteroides) from healthy to advanced adenoma and from advanced adenoma to carcinoma. However, these studies were not able to identify adenoma-associated bacterial communities predictive of cancer progression; this remains a challenge for the future. In addition to the analysis of microbiota in terms of species, studies have been performed to investigate microbiota composition in terms of metabolic capacities. They revealed enrichments in some bacterial functions (xenobiotic metabolism, utilization of polyamines and degradation of polycyclic aromatic compounds) in meta-communities associated with CRC. These functions may lead to the production of pro-tumorigenic metabolites, which are potentially important for cancer development. Thus, gut microbiota could be a new element to consider in the personalized treatment of CRC. The first step is to identify “harmful” bacterial genes so that they or their products can be targeted to limit their deleterious effects.

It has been observed that human CRC biopsies are highly colonized by E. coli. Molecular analyses of these strains have revealed that they frequently harbor in their genome one or several pathogenic islands responsible for the production of toxins. These toxins can induce DNA damage and/or affect the cellular cycle. E. coli harboring cytotoxic necrotizing factor (Cnf) and cytolethal distending toxin (Cdt) are significantly associated with CRC biopsies. However, the toxin most frequently associated with E. coli colonizing CRC is colibactin. Colibactin is a genotoxic polyketide non-ribosomal peptide (PK-NRP) not yet purified that is synthesized by the pks genomic island. Colibactin-producing E. coli (E. coli clb+) increased the number of tumors in different CRC mouse models upon innoculation. E. coli clb+ induced DNA damage such as interstrand crosslinks, which leads to double-strand breaks, cell cycle arrest, and cellular senescence. Senescent cells produce a senescence-associated secretory phenotype (SASP) comprising cytokines, chemokines and growth factors, particularly hepatocyte growth factor (HGF), a marker of poor prognosis in CRC that was involved in the growth of human tumor xenografts in nude mice transiently infected with colibactin-producing E. coli. For all these reasons, targeting colibactin synthesis could be of interest in reducing the impact of E. coli clb+ on CRC development. The role of ClbP peptidase in colibactin maturation and activation has been demonstrated. Structural studies of this periplasmic protein revealed an active serine site, which is accessible to inhibitors and makes ClbP a potential drug target.

Herein, compounds able to bind to and block the catalytic pocket of ClbP, thereby inhibiting its enzyme activity, are disclosed.

The present disclosure is drawn to novel compounds of formula (A), pharmaceutically acceptable salts thereof, and solvates of any of the foregoing and/or pharmaceutically acceptable salts thereof. Compounds of the present disclosure have surprisingly been found to effectively inhibit the enzymatic activity of ClbP and may therefore be used for treatment or prophylaxis of cancer, such as colorectal cancer.

The present disclosure also relates to pharmaceutical compositions comprising at least one entity chosen from compounds of formula (A), pharmaceutically acceptable salts thereof, and solvates of any of the foregoing. The present disclosure also relates to methods of treatment comprising administering at least one entity chosen from compounds of formula (A), pharmaceutically acceptable salts thereof, or pharmaceutical compositions comprising at least one of the foregoing. In some embodiments, the disclosure provides a method of treating a disease. In some embodiments, the disclosure provides a method of treating a disease or condition such as cancer. In some embodiments, the disclosure provides a method of cancer such as colorectal cancer. In some embodiments, the disclosure provides a method of modulating a cancer marker, such as carbohydrate antigen 19-9 and/or carcinoembryonic antigen.

As used herein, the term “pharmaceutically acceptable salt” refers to a salt that is pharmaceutically acceptable as defined herein and that has the desired pharmacological activity of the parent compound. Non-limiting examples of pharmaceutically acceptable salts include those derived from inorganic acids, non-limiting examples of which include hydrochloric acid, hydrobromic acid, hydroiodic acid, sulfuric acid, nitric acid, and phosphoric acid, and those derived from organic acids, non-limiting examples of which include acetic acid, trifluoroacetic acid, propionic acid, glycolic acid, pyruvic acid, oxalic acid, stearic acid, malic acid, maleic acid, malonic acid, salicylic acid, succinic acid, fumaric acid, tartaric acid, citric acid, benzoic acid, cinnamic acid, mandelic acid, p-toluenesulfonic acid, methanesulfonic acid, ethanesulfonic acid, and lactic acid.

Additional non-limiting examples of pharmaceutically acceptable salts include those formed when an acidic proton in a parent compound is replaced by a metal ion, non-limiting examples of which include an alkali metal ion and an alkaline earth metal ion, and those formed when an acidic proton present in a parent compound is replaced by a ammonium ion, a primary ammonium ion, a secondary ammonium ion, a tertiary ammonium ion, or a quaternary ammonium ion. Non-limiting examples of alkali metals and alkaline earth metals include sodium, potassium, lithium, calcium, aluminum, magnesium, copper, zinc, iron, and manganese. Additional non-limiting examples of pharmaceutically acceptable salts include those comprising one or more counterions and zwitterions.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. The same rule applies for any other ranges described herein, even if the values within the range are not specifically called out in this disclosure.

The term “compound,” as used herein unless otherwise indicated, refers to a collection of molecules having an identical chemical structure as a collection of stereoisomers (for example, a collection of racemates, a collection of cis/trans stereoisomers, or a collection of (E) and (Z) stereoisomers). Therefore, geometric and conformational mixtures of the present compounds and salts are within the scope of the disclosure. Unless otherwise stated, all tautomeric forms of the compounds of the disclosure are within the scope of the disclosure.

“Stereoisomer” as used herein refers to enantiomers and diastereomers.

The term “tautomer,” as used herein, refers to one of two or more isomers of a compound that exist together in equilibrium, and are readily interchanged by migration of an atom or group within the molecule.

Unless indicated otherwise, nomenclature used to describe chemical groups or moieties as used herein follow the convention where, reading the name from left to right, the point of attachment to the rest of the molecule is at the right-hand side of the name. For example, the group “(C₁₋₃ alkoxy)C₁₋₃ alkyl,” is attached to the rest of the molecule at the alkyl end. Further examples include methoxyethyl, where the point of attachment is at the ethyl end, and methylamino, where the point of attachment is at the amine end.

Unless indicated otherwise, where a chemical group is described by its chemical formula or structure having a terminal bond moiety indicated by “-”, it will be understood that the “-” represents the point of attachment. In some embodiments, a wavy line (i.e., [symbol] ) depicts the point of attachment.

As used herein, an “acyl” or “alkanoyl” is a functional group with formula RCO— where R is bound to the carbon atom of the carbonyl functional group by a single bond and the “-” denotes the point of attachment to the rest of the molecule. Non-limiting examples of acyls include formyl (HC(O)—, also called methanoyl), acetyl (CH₃C(O)—, also called ethanoyl), and benzoyl (PhC(O)—).

The term “alkyl” or “aliphatic” as used herein, means a straight-chain (i.e., unbranched) or branched, substituted or unsubstituted hydrocarbon chain that is completely saturated and that has a single point of attachment to the rest of the molecule. Unless otherwise specified, an alkyl group is a hydrocarbon chain of 1 to 20 alkyl carbon atoms. In some embodiments, an alkyl group contains one to fourteen carbon atoms (C₁-C₁₄). In some embodiments, an alkyl group contains one to eight carbon atoms (C₁-C₈). In some embodiments, an alkyl group contains one to six carbon atoms (C₁-C₆). In some embodiments, an alkyl group contains one to four carbon atoms (C₁-C₄). In some embodiments, a cyclic alkyl group contains three to six carbon atoms (C₃-C₆). Non-limiting examples of substituted and unsubstituted linear, branched, and cyclic alkyl groups include methyl, ethyl, n-propyl, iso-propyl, cyclopropyl, n-butyl, sec-butyl, iso-butyl, tert-butyl, cyclobutyl, cyclopentyl, cyclohexyl, heptyl, cycloheptyl, octyl, cyclooctyl, nonyl, cyclonony, decyl, cyclodecyl, hydroxymethyl, chloromethyl, fluoromethyl, trifluoromethyl, aminomethyl, 2-aminoethyl, 3-aminopropyl, 4-aminobutyl, dimethylaminomethyl, 2-dimethylaminoethyl, 3-dimethylaminopropyl, 4-dimethylaminobutyl, cyclopropylmethyl, cyclobutylmethyl, cyclopentylmethyl, trifluoroethyl, and trifluoropropyl.

“Alkoxy,” as used herein, refers to an alkyl group, as previously defined, attached to the principal carbon chain through an oxygen (“alkoxy”) atom.

“Halo” and “halogen,” as used herein, are interchangeable and refer to halogen atoms such as fluoro (F), chloro (Cl), bromo (Br), and iodo (I).

“Haloalkyl” refers to an alkyl group substituted with one or more halo atoms (F, Cl, Br, I). For example, “fluoromethyl” refers to a methyl group substituted with one or more fluoro atoms (e.g., monofluoromethyl, difluoromethyl, or trifluoromethyl).

“Haloalkoxy” refers to an alkoxy group substituted with one or more halo atoms (F, Cl, Br, I). For example, “fluoromethoxy” refers to a methoxy group substituted with one or more fluoro atoms (e.g., monofluoromethoxy, difluoromethoxy, or trifluoromethoxy).

“Hydroxyalkyl” refers to an alkyl group substituted with one or more hydroxy groups (—OH).

The terms “cycloalkyl” and “cycloalkyl group” as used interchangeably herein refer to a cyclic saturated monovalent hydrocarbon radical of three to twelve carbon atoms that has a single point of attachment to the rest of the molecule. Cycloalkyl groups may be unsubstituted or substituted. In some embodiments, a cycloalkyl group comprises three to eight carbon atoms (C₃-C₈). In some embodiments, a cycloalkyl group comprises three to six carbon atoms (C₃-C₆). Non-limiting examples of substituted and unsubstituted cycloalkyls include cyclopropyl, cyclopropylmethyl, cyclobutyl, cyclobutylmethyl, cyclopentyl, cyclopentylmethyl, cyclohexyl, cycloheptyl, and cyclooctyl.

The terms “alkylene” and “alkylene group” as used interchangeably herein refer to a saturated divalent (i.e., having two points of attachment to the rest of the molecule) hydrocarbon radical comprising one to twelve carbon atoms (C₁-C₁₂). Alkylene groups may be linear, branched, or cyclic. Alkylene groups may be unsubstituted or substituted. In some embodiments, an alkylene group comprises one to eight carbon atoms (C₁-C₈). In some embodiments, an alkylene group comprises one to six carbon atoms (C₁-C₆). In some embodiments, an alkylene group comprises one to four carbon atoms (C₁-C₄). Non-limiting examples of alkylene groups include methylene and ethylene.

The terms “alkenyl” and “alkenyl group” as used interchangeably herein refer to a monovalent (i.e., having a single point of attachment to the rest of the molecule) hydrocarbon radical comprising two to eight carbon atoms (C₂-C₈) with at least one site of unsaturation (i.e., an sp2 carbon-carbon double bond). Alkenyl groups may be linear, branched, or cyclic. Alkenyl groups may be unsubstituted or substituted. In some embodiments, an alkenyl group contains two to six carbon atoms (C₂-C₆). In some embodiments, an alkenyl group contains two to four carbon atoms (C₂-C₄). Alkenyl groups may have E or Z orientations. Non-limiting examples of alkenyl groups include ethenyl (also called vinyl), 1-propenyl, iso-propenyl, and 2-chloroethenyl.

The terms “alkenylene” and “alkenylene group” as used interchangeably herein refer to a divalent (i.e., having two points of attachment to the rest of the molecule) hydrocarbon radical of two to eight carbon atoms (C₂-C₈) with at least one site of unsaturation (e.g., an sp2 carbon-carbon double bond). Alkenylene groups may be linear, branched, or cyclic. Alkenylene groups may be unsubstituted or substituted. In some embodiments, an alkylene group contains two to six carbon atoms (C₂-C₆). In some embodiments, an alkylene group contains two to four carbon atoms (C₂-C₄). Alkylene groups may have E or Z orientations. A non-limiting example of an alkenyl group is ethenylene (also called vinylene).

The terms “alkynyl” and “alkynyl group” as used interchangeably herein refer to a monovalent (i.e., having a single point of attachment to the rest of the molecule) hydrocarbon radical of two to eight carbon atoms (C₂-C₈) with at least one site of unsaturation (i.e., an sp carbon-carbon triple bond). Alkynyl groups may be linear or branched. Alkynyl groups may be unsubstituted or substituted. In some embodiments, an alkynyl group contains two to six carbon atoms (C₂-C₆). In some embodiments, an alkynyl group contains two to four carbon atoms (C₂-C₄). A non-limiting example of an alkynyl group is ethynyl.

The terms “alkynylene” and “alkynylene group” as used interchangeably herein refer to a divalent (i.e., having two points of attachment to the rest of the molecule) hydrocarbon radical of two to eight carbon atoms (C₂-C₈) with at least one site of unsaturation (i.e., an sp carbon-carbon triple bond). Alkynylene groups may be linear or branched. Alkynylene groups may be unsubstituted or substituted. In some embodiments, an alkynylene group contains two to six carbon atoms (C₂-C₆). In some embodiments, an alkynylene group contains two to four carbon atoms (C₂-C₄). A non-limiting example of an alkynylene group is ethynylene.

As used herein, “aromatic groups” or “aromatic rings” refer to chemical groups that contain conjugated, planar ring systems with delocalized pi electron orbitals comprised of [4n+2]p orbital electrons, wherein n is an integer ranging from 0 to 6. Non-limiting examples of aromatic groups include aryl and heteroaryl groups.

The terms “aryl” and “aryl group” as used interchangeably herein refer to a monovalent (i.e., having a single point of attachment to the rest of the molecule) aromatic hydrocarbon radical of 6-20 carbon atoms (C₆-C₂₀). Aryl groups can be unsubstituted or substituted. Non-limiting examples of unsubstituted and substituted aryl groups include phenyl, 2-fluorophenyl, 3-fluorophenyl, 4-fluorophenyl, 2-methylphenyl, 3-methylphenyl, 4-methylphenyl, 2-chlorophenyl, 3-chlorophenyl, 4-chlorophenyl, 2,6-dichlorophenyl, 3,4-difluorophenyl, 2-hydroxyphenyl, 3-hydroxyphenyl, 4-hydroxyphenyl, 2-methoxyphenyl, 3-methoxyphenyl, 4-methoxyphenyl, 2-phenoxyphenyl, 3-phenoxyphenyl, 4-phenoxyphenyl, 2-cyanophenyl, 3-cyanophenyl, 4-cyanophenyl, 2-dimethylaminophenyl, 3-dimethylaminophenyl, 4-dimethylaminophenyl, 3-methylsulfonylphenyl, 4-methylsulfonylphenyl, 3-aminophenyl, 3-methylaminophenyl, 3-(2-hydroxyethoxy)phenyl, 2-trifluoromethylphenyl, 3-trifluoromethylphenyl, 4-trifluoromethylphenyl, 2-isopropylphenyl, 3-isopropylphenyl, 4-isopropylphenyl, 1-naphthyl and 2-naphthyl.

The term “heteroalkyl” as used herein refers to an alkyl group wherein at least one of the carbon atoms in the chain is replaced by a heteroatom, such as nitrogen, oxygen, phosphorous, and sulfur. A heteroalkyl group may be unsubstituted or substituted.

The terms “heterocycloalkyl,” “heterocycle,” “heterocyclyl,” and “heterocyclic group” as used interchangeably herein refer to a saturated or partially unsaturated ring system of 3 to 20 atoms, wherein at least one of the ring atoms is a heteroatom, such as nitrogen, oxygen, phosphorous, and sulfur. A heterocycloalkyl group may be unsubstituted or substituted. In some embodiments, a heterocycloalkyl group comprises 3 to 10 atoms. In some embodiments, a heterocycloalkyl group contains 3 to 7 atoms. In some embodiments, a heterocycloalkyl group is monocyclic. In some embodiments, a heterocycloalkyl group is bicyclic. In some embodiments, a heterocycloalkyl group comprises fused rings. Non-limiting examples of unsubstituted and substituted heterocycloalkyl groups include pyrrolidinyl, N-methylpyrrolidinyl, azetidinyl, dihydrofuranyl, tetrahydrofuranyl, tetrahydropyranyl, 3-hydroxypyrrolidinyl, 3-methoxypyrrolidinyl, and benzodioxolyl.

The terms “heteroaryl” and “heteroaryl group” as used interchangeably herein refer to an aromatic ring system of 5 to 10 atoms, wherein at least one of the ring atoms is a heteroatom, such as nitrogen, oxygen, phosphorous, and sulfur. A heteroaryl group may be unsubstituted or substituted. In some embodiments, a heteroaryl group contains 5 to 10 atoms. In some embodiments, a heteroaryl group contains 5 to 9 atoms. In some embodiments, a heteroaryl group contains 5 atoms. In some embodiments, a heteroaryl group contains 6 atoms. In some embodiments, a heteroaryl group contains 7 atoms. In some embodiments, a heteroaryl group is monocyclic. In some embodiments, a heteroaryl group is bicyclic. In some embodiments, a heteroaryl group contains fused rings. Non-limiting examples of heteroaryl groups include pyridinyl, imidazolyl, imidazopyridinyl, pyrimidinyl, pyrazolyl, triazolyl, pyrazinyl, tetrazolyl, furyl, 2-thienyl, 3-thienyl, isoxazolyl, thiazolyl, oxadiazolyl, 3-methyl-1,2,4-oxadiazolyl, 3-phenyl-1,2,4-oxadiazolyl, oxazolyl, isothiazolyl, pyrrolyl, quinolinyl, isoquinolinyl, tetrahydroisoquinolinyl, indolyl, benzimidazolyl, benzofuranyl, indazolyl, indolizinyl, phthalazinyl, pyridazinyl, triazinyl, thiadiazolyl, furazanyl, benzofurazanyl, benzothiophenyl, benzothiazolyl, benzoxazolyl, quinazolinyl, quinoxalinyl, naphthyridinyl, furopyridinyl, and 1H-pyrrolo[2,3-b]pyridinyl. Non-limiting examples of heteroaryl groups include:

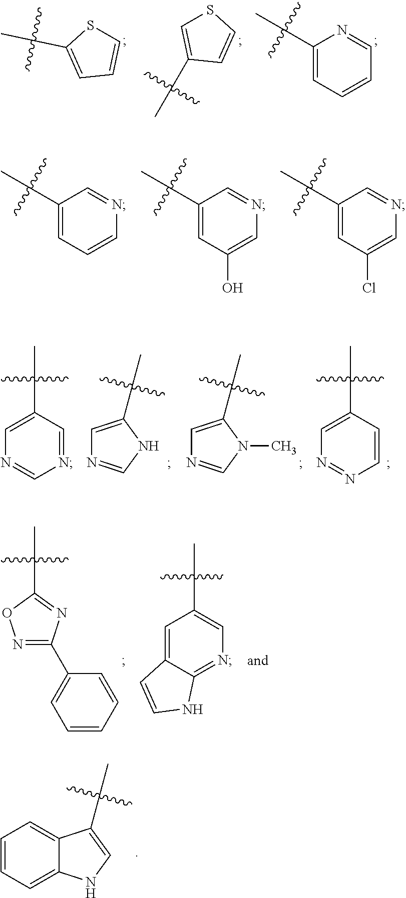

The terms “amino,” “amino group,” and “amine group,” as used interchangeably herein, include primary amine groups, secondary amine groups, and tertiary amine groups, respectively substituted with 0, 1, or 2 non-hydrogen substituents, which may be identical or different, such as alkyl, haloalkyl, alkoxy, cycloalkyl, heterocycloalkyl, aryl, and heteroaryl substituents. Non-limiting examples of amino groups are alkylamine groups, dialkylamine groups, arylamine groups, diarylamine groups, aralkylamine groups, and diaralkylamine groups, such as methylamine, ethylamine, propylamine, isopropylamine, phenylamine, benzylamine, dimethylamine, diethylamine, dipropylamine, diisopropylamine, methylethylamine, methylphenylamine, and methylbenzylamine.

The phrase “optionally substituted” as used herein means may or may not be “substituted.” The term “substituted” as used herein refers to the replacement of one or more hydrogen atoms on a group (such as on an alkyl group, haloalkyl group, alkylene group, alkenyl group, alkenylene group, alkynyl group, alkynylene group, aryl group, heterocycloalkyl group, heteroaryl group, or an amino group) by one or more substituents. Non-limiting examples of substituents that replace a single hydrogen atom include halogen, hydroxyl, and amino. Non-limiting examples of substituents that replace two hydrogen atoms include oxo and methene. Non-limiting examples of substituents that replace three hydrogen atoms include nitrile.

Additional non-limiting examples of substituents include:
C₁-C₁₀ linear, branched, and cyclic alkyl groups, non-limiting examples of which include methyl, ethyl, n-propyl, iso-propyl, cyclopropyl, n-butyl sec-butyl, iso-butyl, tert-butyl, cyclobutyl, cyclopentyl, and cyclohexyl;
C₂-C₈ linear, branched, and cyclic alkenyl groups, non-limiting examples of which include ethenyl (also called vinyl), 1-propenyl, and iso-propenyl;
C₂-C₈ linear and branched alkynyl groups, non-limiting examples of which include ethynyl;
substituted and unsubstituted aryl groups, non-limiting examples of which include phenyl, 2-fluorophenyl, 3-methylphenyl, 4-chlorophenyl, 2,6-dichlorophenyl, 3,4-difluorophenyl, 3-hydroxyphenyl, 4-cyanophenyl, 2-dimethylaminophenyl, 3-methylsulfonylphenyl, 4-trifluoromethylphenyl, 3-isopropylphenyl, 1-naphthyl, and 2-naphthyl;
substituted and unsubstituted heterocyclic groups, non-limiting examples of which include pyrrolidinyl, N-methylpyrrolidinyl, azetidinyl, dihydrofuranyl, tetrahydrofuranyl, tetrahydropyranyl, 3-hydroxypyrrolidinyl, and 3-methoxypyrrolidinyl;
substituted and unsubstituted heteroaryl groups, non-limiting examples of which include pyridinyl, imidazolyl, pyrimidinyl, pyrazolyl, furyl, 2-thienyl, 3-thienyl, isoxazolyl, thiazolyl, oxadiazolyl, 3-methyl-1,2,4-oxadiazolyl, 3-phenyl-1,2,4-oxadiazolyl, indolyl, benzothiazolyl, and 1H-pyrrolo[2,3-b]pyridinyl;
—(CRₐR_{b})_{z}OR_{c}, non-limiting examples of which include —OH, —OCH₃, —OCH₂OH, and —OCH₂CH₃;
—(CRₐR_{b})_{z}N(R_{c})(R_{d}), non-limiting examples of which include —NH₂, —NHCH₃, —N(CH₃)₂, —CH₂NH₂, and —CH₂NHCH₃;
a halogen atom, non-limiting examples of which include a fluorine atom (—F) and a chlorine atom (—Cl);
—(CR^{a}R^{b})_{z}CN;
—(CR^{a}R^{b})_{z}NO₂;
—CHₓX_{y}, wherein X is a halogen atom and x+y sum to 3, non-limiting examples of which include —CH₂F, —CHF₂, and —CF₃;
—(CR^{a}R^{b})_{z}C(O)R^{c}, non-limiting examples of which include —COCH₃, —COCH₂CH₃, and —CH₂COCH₃;
—(CR^{a}R^{b})_{z}C(O)OR^{c}, non-limiting examples of which include —CO₂H, —CO₂CH₃, —CO₂CH₂CH₃, and —CH₂CO₂CH₃;
—(CR^{a}R^{b})_{z}C(O)N(R^{c})(R^{d}), non-limiting examples of which include —CONH₂, —CONHCH₃, —CON(CH₃)₂, —CH₂CONH₂, —CH₂CONHCH₃, —CH₂CON(CH₃)₂;
—(CR^{a}R^{b})_{z}SO₂R^{c}; non-limiting examples of which include —SO₂H, —SO₂CH₃, —CH₂SO₂H, —CH₂SO₂CH₃, —SO₂C₆H₅, and —CH₂SO₂C₆H₅; and
—(CR^{a}R_{b})_{z}SO₃R^{c}; non-limiting examples of which include —SO₂H, —SO₂CH₃, —CH₂SO₂H, —CH₂SO₂CH₃, —SO₂C₆H₅, and —CH₂SO₂C₆H₅; and
wherein each of R^{a} and R^{b} is independently chosen from hydrogen and substituted or unsubstituted C₁-C₆ linear, branched, or cyclic alkyl, each of R^{c} and R^{d} is independently chosen from hydrogen, substituted or unsubstituted C₁-C₆ linear, branched, or cyclic alkyl, and aryl, or wherein R^{c} and R^{d} together form a ring system comprising 3 to 7 atoms, and z is chosen from 0, 1, 2, 3, and 4.

As used herein, the term “pharmaceutical composition” refers to a preparation that is in such form as to permit the biological activity of the active ingredient to be effective, and that contains no additional components that are unacceptably toxic to a subject to which the composition would be administered. In some embodiments, such compositions may be sterile.

The term “pharmaceutically acceptable,” as used herein in “pharmaceutically acceptable salt” and “pharmaceutically acceptable excipient,” refers to a component that is, within the scope of sound medical judgment, suitable for use in contact with the tissues of humans and other mammals without undue toxicity, irritation, allergic response and the like, and are commensurate with a reasonable benefit/risk ratio.

The phrase “pharmaceutically acceptable excipient” is employed herein to refer to a pharmaceutically acceptable material chosen from a solvent, dispersion media, diluent, dispersion, suspension aid, surface active agent, isotonic agent, thickening or emulsifying agent, preservative, polymer, peptide, protein, cell, hyaluronidase, and mixtures thereof. In some embodiments, the solvent is an aqueous solvent.

“Treatment,” “treat,” and “treating” refer to reversing, alleviating (e.g., alleviating one or more symptoms), and/or delaying the progression of a medical condition or disorder described herein.

The terms “disease” and “disorder” are used interchangeably herein and refer to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, sickness, illness, complaint, indisposition, or affection.

“Subject,” as used herein, means an animal subject, such as a mammalian subject, and particularly human beings.

As used herein, the term “administering” refers to the placement of a compound, pharmaceutically acceptable salt thereof, and/or a pharmaceutical composition comprising into a mammalian tissue or a subject by a method or route that results in at least partial localization of the compound, salt, and/or composition at a desired site or tissue location.

The term “therapeutically effective amount” as used herein refers to an amount of a compound or salt that produces a desired effect for which it is administered (e.g., improvement in symptoms of a disease or condition such as cancer, lessening the severity of such a disease or condition or a symptom thereof, and/or reducing progression any one of the foregoing). The exact amount of an effective dose will depend on the purpose of the treatment and will be ascertainable by one skilled in the art using known techniques (see, e.g., Lloyd (1999) The Art, Science and Technology of Pharmaceutical Compounding).

One of ordinary skill in the art would recognize that, when an amount of a compound is disclosed, the relevant amount of a pharmaceutically acceptable salt form of the compound is an amount equivalent to the amount of the free base of the compound. The amounts of the compounds and pharmaceutically acceptable salts disclosed herein are based upon the free base form of the relevant compound. For example, “10 mg of at least one entity chosen from compounds of Formulas I or Ia and pharmaceutically acceptable salts thereof” refers to 10 mg of a compound of Formulas I or Ia or an amount of a pharmaceutically acceptable salt of the compound of Formulas I or Ia equivalent to 10 mg of the relevant compound of Formulas I or Ia.

The “effectiveness” of a compound or composition of the disclosure can be assessed by any method known to one of ordinary skill in the art, including those described in the examples of this disclosure. Effectiveness can be established in vitro (biochemical and/or biological in cultured cells) and/or in vivo. Effectiveness in vitro may be used to extrapolate or predict some degree of effectiveness in vivo, in an animal or in a human subject. A reference or standard or comparison may be used. The term “effective” at inhibiting a receptor (such as ClbP), and/or signaling mediated by the enzyme in the context of this disclosure and claims means reducing/activating the activity of the receptor and/or the activation and propagation of the signaling pathway in terms of activation of a downstream molecule or known biological effect by a detectable or measurable amount relative to the baseline activity. This can be assessed in vitro or in vivo and, in some cases, extrapolated to what an activity or benefit in vivo might be by one of ordinary skill in the art. In some embodiments, the reduction or activation is measured in terms of percentage reduction or activation, relative to the activity in the absence of exposure to the compound of the disclosure, including, for example, at least 5%, at least 10%, 20%, at least 30%, at least 40%, at least 50%, at least 60%, at least 70%, at least 80%, at least 90%, or about 100%. The activity might also fall within a range, e.g., 5-10%, 10-20%, and any other range interval between 1% and 100%. An amount is “effective” in vivo if it produces any benefit to the subject to whom the compound or salt is administered.

Disclosed herein are compounds of Formula (A):

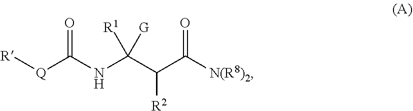

pharmaceutically acceptable salts thereof, and solvates of any of the foregoing, wherein:
R′ is chosen from hydrogen, C₁₋₁₄ alkyl and C₁₋₁₄ alkenyl, wherein alkyl and alkenyl are optionally substituted;
Q is CR″R″′, NR″, or O;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl; and
G is chosen from C(═O)CF₃, C(═O)H, B(OH)₂, B(Oalkyl)₂, CN, P(═O)(Oaryl)₂, P(═O)(OCH₃)F, S(═O)₂F, C(═O)aryl, C(═O)heteroaryl,

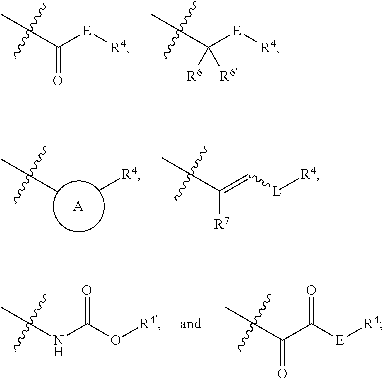

wherein alkyl, aryl, and heteroaryl are optionally substituted;
wherein:
E is chosen from CR³R³′, NR³, and O, wherein optionally R¹ and R³ or R² and R³ together form a ring;
L is chosen from S(═O)₂, C(═O), C(═O)NR³, and a bond;
R³ and R^{3′} each independently is chosen from H, halogen, and C₁₋₃ alkyl;
Ring A is chosen from 4-8 membered heteroaryl and 4-8 membered heterocyclyl; wherein the 4-8 membered heteroaryl or 4-8 membered heterocyclyl is optionally substituted;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted;
R^{4′} is chosen from C₁₋₇ alkyl and C₀₋₆ alkyl-R⁵; wherein the C₁₋₇ alkyl or C₀₋₆ alkyl-R⁵ is optionally substituted;
R⁵ is chosen from C₆₋₁₀ aryl, 5-10 membered heteroaryl, 3-10 membered cycloalkyl, 3-10 membered heterocycloalkyl and C(═O)O—C₁₋₆ alkyl; wherein the C₆₋₁₀ aryl, 5-10 membered heteroaryl, 3-10 membered heterocycloalkyl, 3-10 membered cycloalkyl, and C(═O)O—C₁₋₆ alkyl are optionally substituted;
R⁶ and R^{6′} each independently is chosen from H, OH, haloalkyl, and C₁₋₃ alkyl, or R⁶ and R^{6′} together form a 4-6 membered ring; and
R⁷ is chosen from H, C₁₋₃ alkyl, and halogen.

Disclosed herein are compounds of Formula (I):

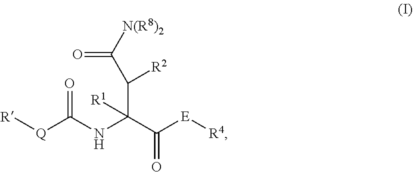

pharmaceutically acceptable salts thereof, and solvates of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
E is chosen from CR³R³′, NR³, and O, wherein optionally R¹ and R³ or R² and R³ together form a ring;
R³ and R^{3′} each independently is chosen from H, halogen, and C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted; and
R⁵ is chosen from C₆₋₁₀ aryl, 5-10 membered heteroaryl, 3-10 membered cycloalkyl, 3-10 membered heterocycloalkyl and C(═O)O—C₁₋₆ alkyl; wherein the C₆₋₁₀ aryl, 5-10 membered heteroaryl, 3-10 membered heterocycloalkyl, 3-10 membered cycloalkyl, and C(═O)O—C₁₋₆ alkyl are optionally substituted.

Disclosed herein are compounds of Formula (II):

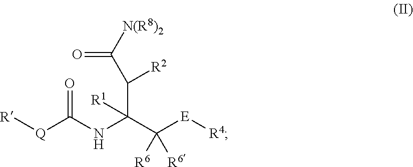

pharmaceutically acceptable salts thereof, and solvates of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
E is chosen from CR³R³′, NR³, and O, wherein optionally R¹ and R³ or R² and R³ together form a ring;
R³ and R^{3′} each independently is chosen from H, halogen, or C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl, and O-aryl; wherein the C₁₋₇ alkyl, C₁₋₆ alkyl-R⁵, C(═O)—C₀₋₆ alkyl-R⁵, O-alkyl or O-aryl is optionally substituted;
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted; and
R⁶ and R^{6′} each independently is chosen from H, OH, haloalkyl, and C₁₋₃ alkyl, or R⁶ and R^{6′} together form a 4-6 membered ring.

Disclosed herein are compounds of Formula (III):

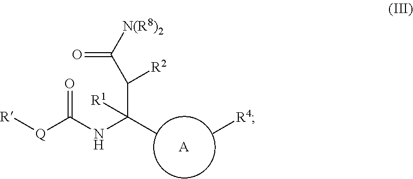

pharmaceutically acceptable salts thereof, and solvates of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
Ring A is chosen from 4-8 membered heteroaryl and 4-8 membered heterocyclyl; wherein the 4-8 membered heteroaryl or 4-8 membered heterocyclyl is optionally substituted;
R⁴ is chosen from C₁₋₇ alkyl, C₁₋₆ alkyl-R⁵, C(═O)—C₀₋₆ alkyl-R⁵, Cl, F, NH₂, O-alkyl, and O-aryl; wherein the C₁₋₇ alkyl, C₁₋₆ alkyl-R⁵, C(═O)—C₀₋₆ alkyl-R⁵, O-alkyl or O-aryl is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.

Disclosed herein are compounds of Formula (IV):

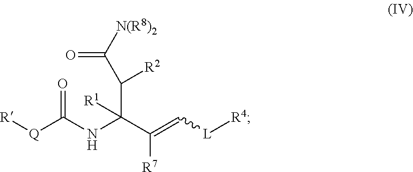

pharmaceutically acceptable salts thereof, and solvates of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
L is chosen from S(═O)₂, C(═O), C(═O)NR³, and a bond;
R³ and R^{3′} each independently is chosen from H, halogen, and C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted;
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted; and
R⁷ is chosen from H, C₁₋₃ alkyl, and halogen.

Disclosed herein are compounds of Formula (V):

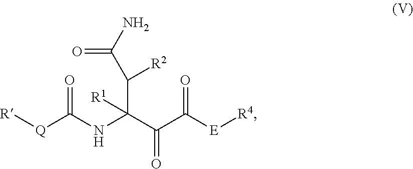

pharmaceutically acceptable salts thereof, and solvates of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
E is chosen from CR³R³′, NR³, and O, wherein optionally R¹ and R³ or R² and R³ together form a ring;
R³ and R^{3′} each independently is chosen from H, halogen, and C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.

Disclosed herein are compounds of Formula (VI):

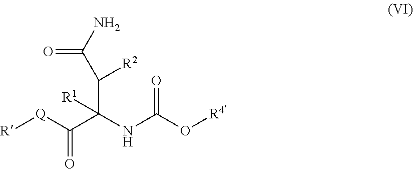

pharmaceutically acceptable salts thereof, and solvates of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
R^{4′} is chosen from C₁₋₇ alkyl and C₀₋₆ alkyl-R⁵; wherein the C₁₋₇ alkyl or C₀₋₆ alkyl-R⁵ is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.

In some embodiments, the present disclosure is drawn to one or more compounds recited in Table 1.

TABLE 1

| | |
|---|---|
| 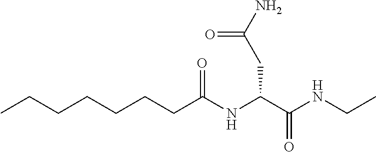 [SMILES: CCCCCCCC(=O)N[C@H](CC(N)=O)C(=O)NCC] | Compound I |
| 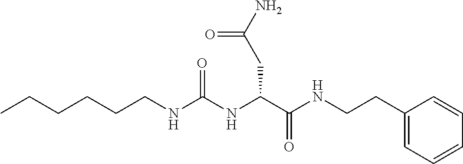 [SMILES: CCCCCCNC(=O)N[C@H](CC(N)=O)C(=O)NCCc1ccccc1] | Compound II |
| 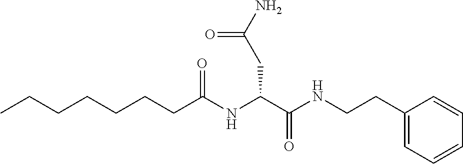 [SMILES: CCCCCCCC(=O)N[C@H](CC(N)=O)C(=O)NCCc1ccccc1] | Compound III |
| 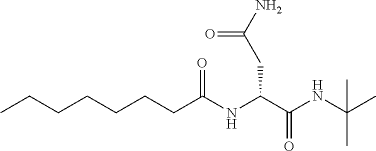 [SMILES: CCCCCCCC(=O)N[C@H](CC(N)=O)C(=O)NC(C)(C)C] | Compound IV |
| 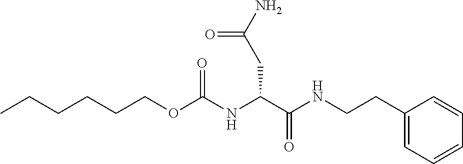 [SMILES: CCCCCCOC(=O)N[C@H](CC(N)=O)C(=O)NCCc1ccccc1] | Compound V |
| 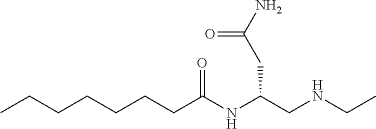 [SMILES: CCCCCCCC(=O)N[C@@H](CNCC)CC(N)=O] | Compound VI |
| 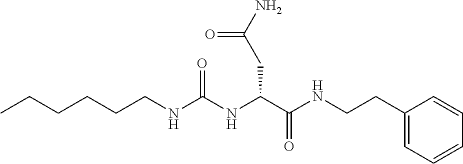 [SMILES: CCCCCCNC(=O)N[C@H](CC(N)=O)C(=O)NCCc1ccccc1] | Compound VII |
| 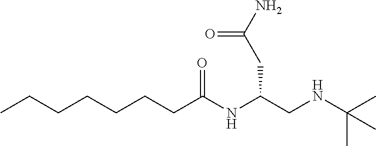 [SMILES: CCCCCCCC(=O)N[C@@H](CNC(C)(C)C)CC(N)=O] | Compound VIII |
| 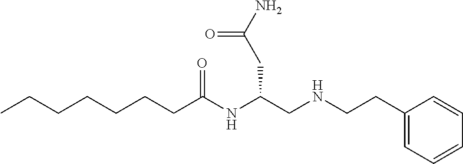 [SMILES: CCCCCCCC(=O)N[C@@H](CNCCc1ccccc1)CC(N)=O] | Compound IX |
| 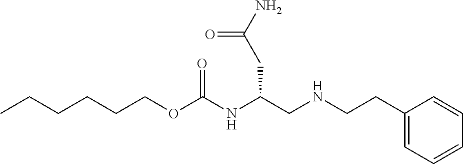 [SMILES: CCCCCCOC(=O)N[C@@H](CNCCc1ccccc1)CC(N)=O] | Compound X |
| 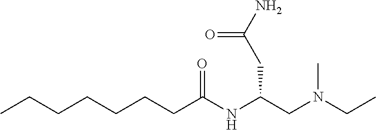 [SMILES: CCCCCCCC(=O)N[C@H](CC(N)=O)CN(C)CC] | Compound XI |
| 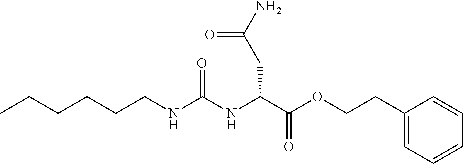 [SMILES: CCCCCCNC(=O)N[C@H](CC(N)=O)C(=O)OCCc1ccccc1] | Compound XII |
| 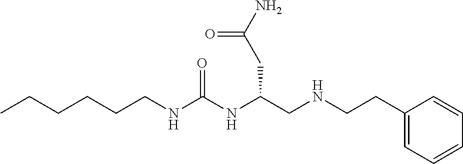 [SMILES: CCCCCCNC(=O)N[C@@H](CNCCc1ccccc1)CC(N)=O] | Compound XIII |
| 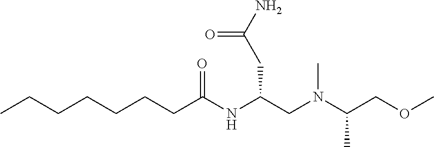 [SMILES: CCCCCCCC(=O)N[C@H](CC(N)=O)CN(C)[C@@H](C)COC] | Compound XIV |
| 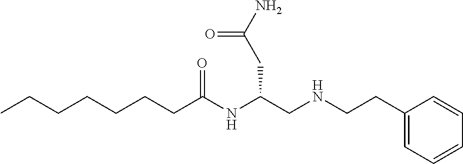 [SMILES: CCCCCCCC(=O)N[C@@H](CNCCc1ccccc1)CC(N)=O] | Compound XV |
| 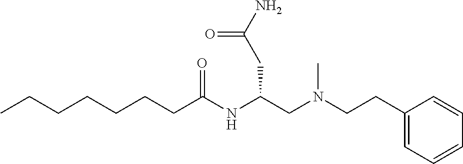 [SMILES: CCCCCCCC(=O)N[C@H](CC(N)=O)CN(C)CCc1ccccc1] | Compound XVI |
| 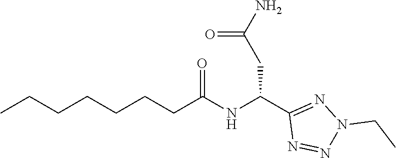 [SMILES: CCCCCCCC(=O)N[C@H](CC(N)=O)c1nnn(CC)n1] | Compound XVII |
| 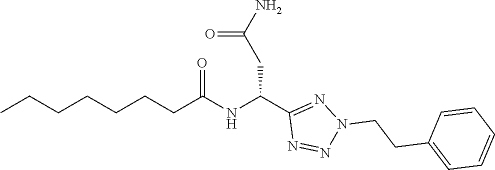 [SMILES: CCCCCCCC(=O)N[C@H](CC(N)=O)c1nnn(CCc2ccccc2)n1] | Compound XVIII |
| 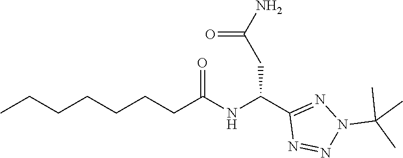 [SMILES: CCCCCCCC(=O)N[C@H](CC(N)=O)c1nnn(C(C)(C)C)n1] | Compound XIX |
| 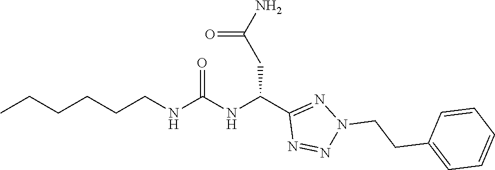 [SMILES: CCCCCCNC(=O)N[C@H](CC(N)=O)c1nnn(CCc2ccccc2)n1] | Compound XX |
| 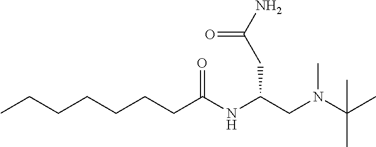 [SMILES: CCCCCCCC(=O)N[C@H](CC(N)=O)CN(C)C(C)(C)C] | Compound XXI |
| 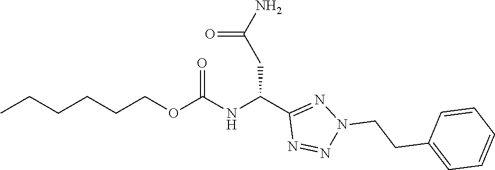 [SMILES: CCCCCCOC(=O)N[C@H](CC(N)=O)c1nnn(CCc2ccccc2)n1] | Compound XXII |
| 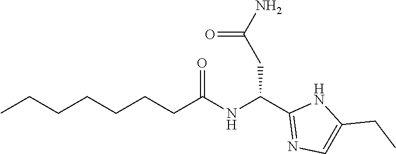 [SMILES: CCCCCCCC(=O)N[C@H](CC(N)=O)c1ncc(CC)[nH]1] | Compound XXIII |
| 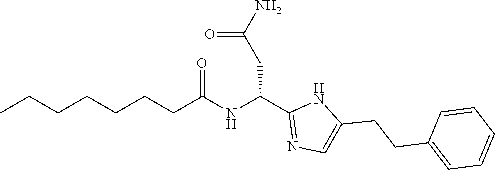 [SMILES: CCCCCCCC(=O)N[C@H](CC(N)=O)c1ncc(CCc2ccccc2)[nH]1] | Compound XXIV |
| 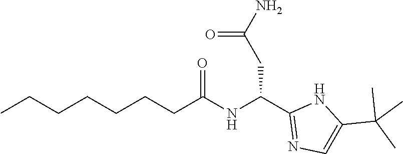 [SMILES: CCCCCCCC(=O)N[C@H](CC(N)=O)c1ncc(C(C)(C)C)[nH]1] | Compound XXV |
| 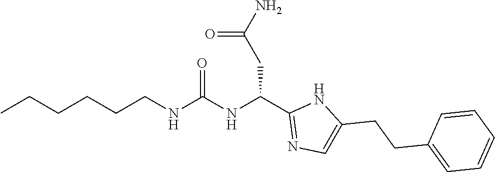 [SMILES: CCCCCCNC(=O)N[C@H](CC(N)=O)c1ncc(CCc2ccccc2)[nH]1] | Compound XXVI |
| 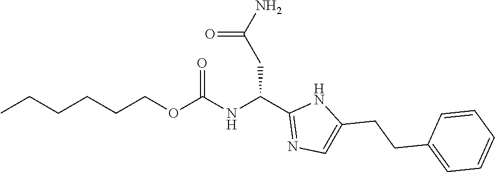 [SMILES: CCCCCCOC(=O)N[C@H](CC(N)=O)c1ncc(CCc2ccccc2)[nH]1] | Compound XXVII |
| 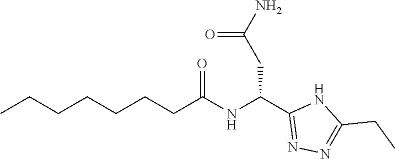 [SMILES: CCCCCCCC(=O)N[C@H](CC(N)=O)c1nnc(CC)[nH]1] | Compound XXVIII |
| 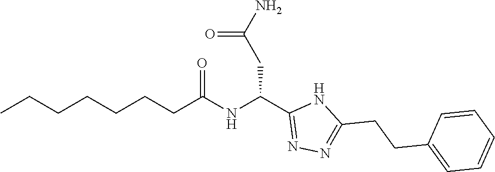 [SMILES: CCCCCCCC(=O)N[C@H](CC(N)=O)c1nnc(CCc2ccccc2)[nH]1] | Compound XXIX |
| 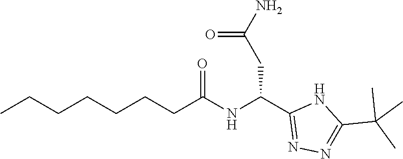 [SMILES: CCCCCCCC(=O)N[C@H](CC(N)=O)c1nnc(C(C)(C)C)[nH]1] | Compound XXX |
| 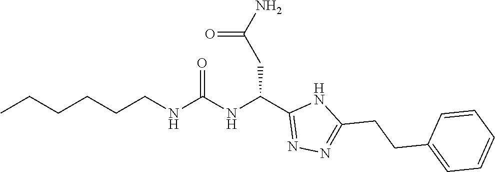 [SMILES: CCCCCCNC(=O)N[C@H](CC(N)=O)c1nnc(CCc2ccccc2)[nH]1] | Compound XXXI |
| 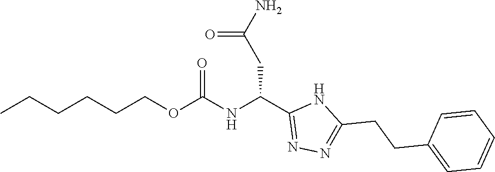 [SMILES: CCCCCCOC(=O)N[C@H](CC(N)=O)c1nnc(CCc2ccccc2)[nH]1] | Compound XXXII |
| 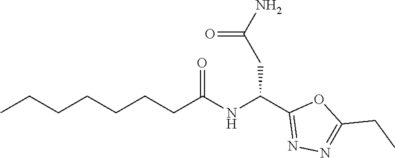 [SMILES: CCCCCCCC(=O)N[C@H](CC(N)=O)c1nnc(CC)o1] | Compound XXXIII |
| 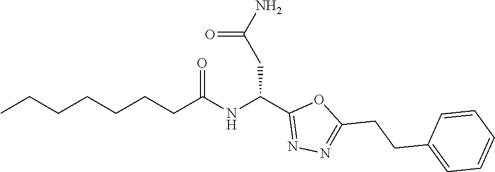 [SMILES: CCCCCCCC(=O)N[C@H](CC(N)=O)c1nnc(CCc2ccccc2)o1] | Compound XXXIV |
| 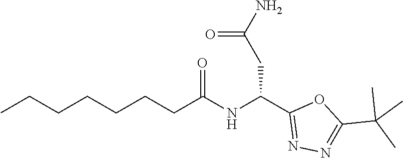 [SMILES: CCCCCCCC(=O)N[C@H](CC(N)=O)c1nnc(C(C)(C)C)o1] | Compound XXXV |
| 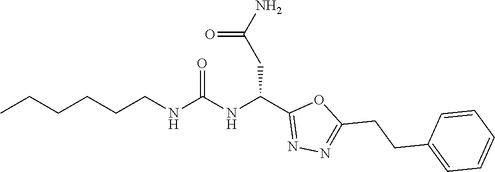 [SMILES: CCCCCCNC(=O)N[C@H](CC(N)=O)c1nnc(CCc2ccccc2)o1] | Compound XXXVI |
| 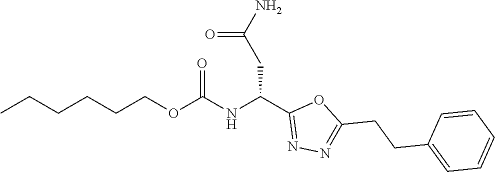 [SMILES: CCCCCCOC(=O)N[C@H](CC(N)=O)c1nnc(CCc2ccccc2)o1] | Compound XXXVII |
| 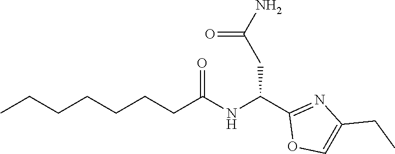 [SMILES: CCCCCCCC(=O)N[C@H](CC(N)=O)c1nc(CC)co1] | Compound XXXVIII |
| 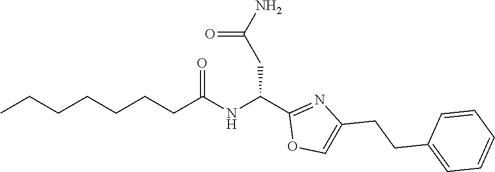 [SMILES: CCCCCCCC(=O)N[C@H](CC(N)=O)c1nc(CCc2ccccc2)co1] | Compound XXXIX |
| 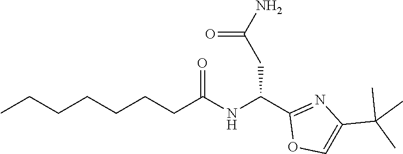 [SMILES: CCCCCCCC(=O)N[C@H](CC(N)=O)c1nc(C(C)(C)C)co1] | Compound XL |
| 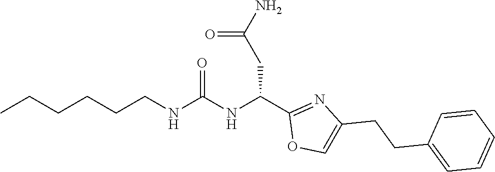 [SMILES: CCCCCCNC(=O)N[C@H](CC(N)=O)c1nc(CCc2ccccc2)co1] | Compound XLI |
| 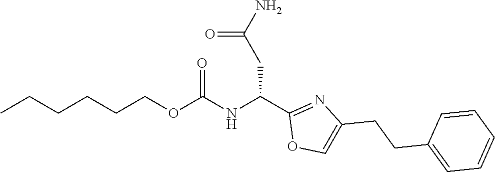 [SMILES: CCCCCCOC(=O)N[C@H](CC(N)=O)c1nc(CCc2ccccc2)co1] | Compound XLII |
| 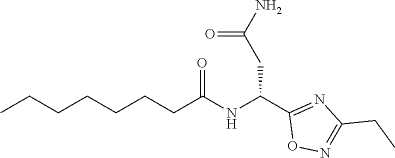 [SMILES: CCCCCCCC(=O)N[C@H](CC(N)=O)c1nc(CC)no1] | Compound XLIII |
| 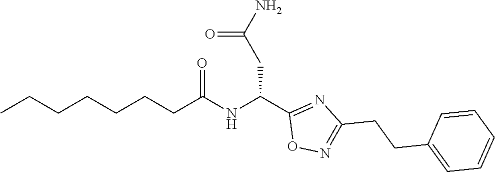 [SMILES: CCCCCCCC(=O)N[C@H](CC(N)=O)c1nc(CCc2ccccc2)no1] | Compound XLIV |
| 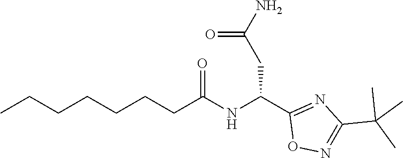 [SMILES: CCCCCCCC(=O)N[C@H](CC(N)=O)c1nc(C(C)(C)C)no1] | Compound XLV |
| 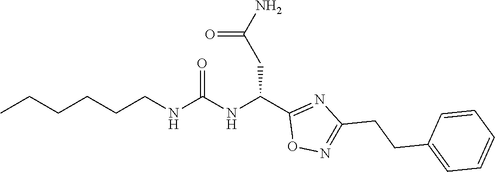 [SMILES: CCCCCCNC(=O)N[C@H](CC(N)=O)c1nc(CCc2ccccc2)no1] | Compound XLVI |
| 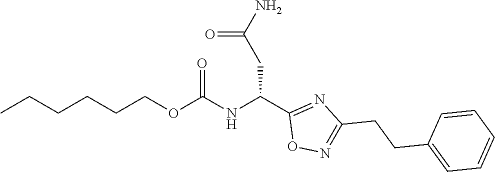 [SMILES: CCCCCCOC(=O)N[C@H](CC(N)=O)c1nc(CCc2ccccc2)no1] | Compound XLVII |
| 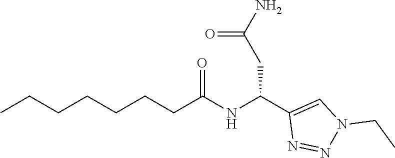 [SMILES: CCCCCCCC(=O)N[C@H](CC(N)=O)c1cn(CC)nn1] | Compound XLVIII |
| 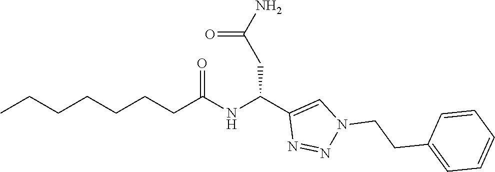 [SMILES: CCCCCCCC(=O)N[C@H](CC(N)=O)c1cn(CCc2ccccc2)nn1] | Compound XLIX |
| 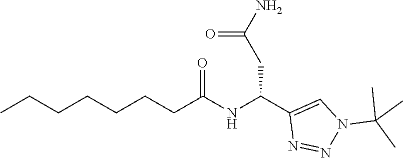 [SMILES: CCCCCCCC(=O)N[C@H](CC(N)=O)c1cn(C(C)(C)C)nn1] | Compound L |
| 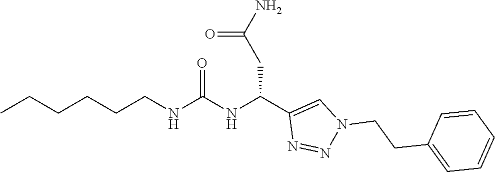 [SMILES: CCCCCCNC(=O)N[C@H](CC(N)=O)c1cn(CCc2ccccc2)nn1] | Compound LI |
| 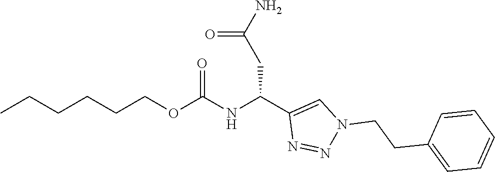 [SMILES: CCCCCCOC(=O)N[C@H](CC(N)=O)c1cn(CCc2ccccc2)nn1] | Compound LII |
| 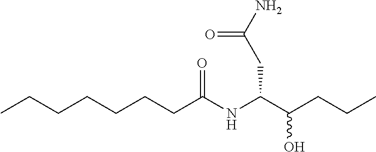 [SMILES: CCCCCCCC(=O)N[C@H](CC(N)=O)C(O)CCC] | Compound LIII |
| 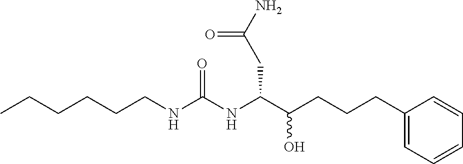 [SMILES: CCCCCCNC(=O)N[C@H](CC(N)=O)C(O)CCCc1ccccc1] | Compound LIV |
| 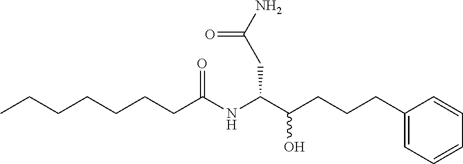 [SMILES: CCCCCCCC(=O)N[C@H](CC(N)=O)C(O)CCCc1ccccc1] | Compound LV |
| 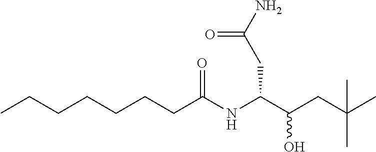 [SMILES: CCCCCCCC(=O)N[C@H](CC(N)=O)C(O)CC(C)(C)C] | Compound LVI |
| 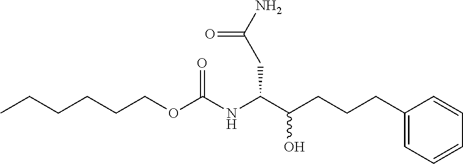 [SMILES: CCCCCCOC(=O)N[C@H](CC(N)=O)C(O)CCCc1ccccc1] | Compound LVII |
| 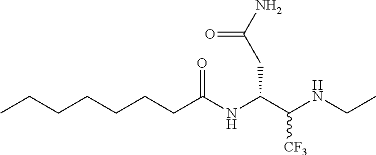 [SMILES: CCCCCCCC(=O)N[C@H](CC(N)=O)C(NCC)C(F)(F)F] | Compound LVIII |
| 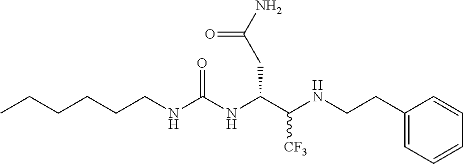 [SMILES: CCCCCCNC(=O)N[C@H](CC(N)=O)C(NCCc1ccccc1)C(F)(F)F] | Compound LIX |
| 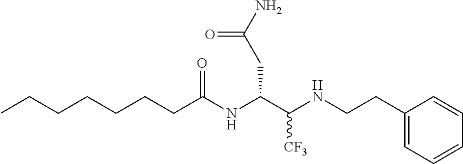 [SMILES: CCCCCCCC(=O)N[C@H](CC(N)=O)C(NCCc1ccccc1)C(F)(F)F] | Compound LX |
| 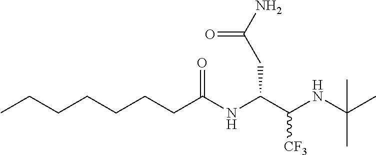 [SMILES: CCCCCCCC(=O)N[C@H](CC(N)=O)C(NC(C)(C)C)C(F)(F)F] | Compound LXI |
| 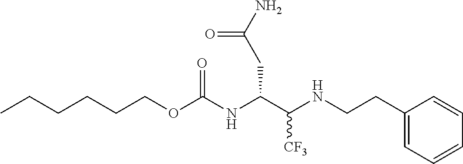 [SMILES: CCCCCCOC(=O)N[C@H](CC(N)=O)C(NCCc1ccccc1)C(F)(F)F] | Compound LXII |
| 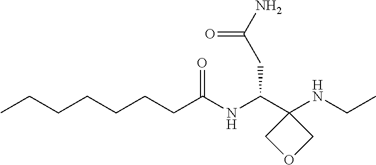 [SMILES: CCCCCCCC(=O)N[C@H](CC(N)=O)C1(NCC)COC1] | Compound LXIII |
| 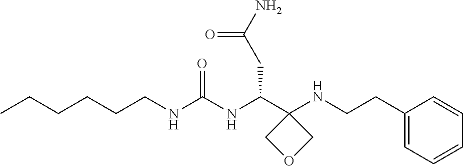 [SMILES: CCCCCCNC(=O)N[C@H](CC(N)=O)C1(NCCc2ccccc2)COC1] | Compound LXIV |
| 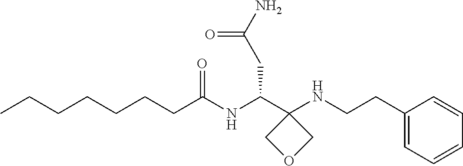 [SMILES: CCCCCCCC(=O)N[C@H](CC(N)=O)C1(NCCc2ccccc2)COC1] | Compound LXV |
| 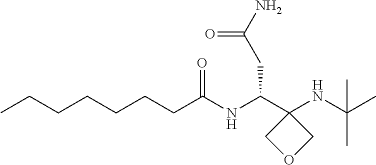 [SMILES: CCCCCCCC(=O)N[C@H](CC(N)=O)C1(NC(C)(C)C)COC1] | Compound LXVI |
| 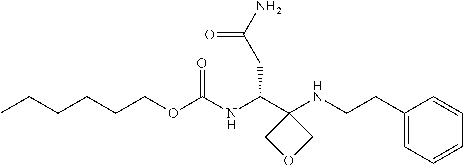 [SMILES: CCCCCCOC(=O)N[C@H](CC(N)=O)C1(NCCc2ccccc2)COC1] | Compound LXVII |
| 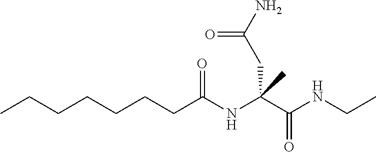 [SMILES: CCCCCCCC(=O)N[C@](C)(CC(N)=O)C(=O)NCC] | Compound LXVIII |
| 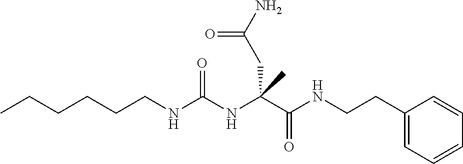 [SMILES: CCCCCCNC(=O)N[C@](C)(CC(N)=O)C(=O)NCCc1ccccc1] | Compound LXIX |
| 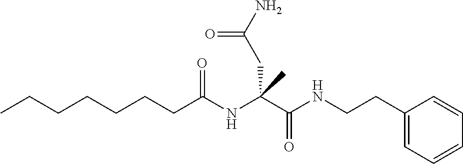 [SMILES: CCCCCCCC(=O)N[C@](C)(CC(N)=O)C(=O)NCCc1ccccc1] | Compound LXX |
| 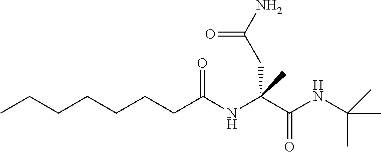 [SMILES: CCCCCCCC(=O)N[C@](C)(CC(N)=O)C(=O)NC(C)(C)C] | Compound LXXI |
| 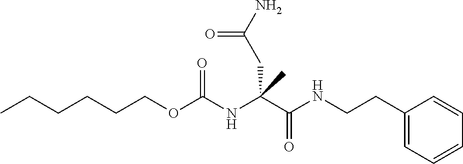 [SMILES: CCCCCCOC(=O)N[C@](C)(CC(N)=O)C(=O)NCCc1ccccc1] | Compound LXXII |
| 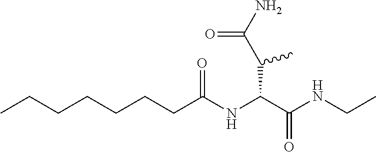 [SMILES: CCCCCCCC(=O)N[C@@H](C(=O)NCC)C(C)C(N)=O] | Compound LXXIII |
| 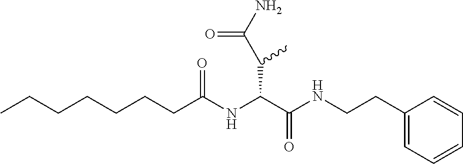 [SMILES: CCCCCCCC(=O)N[C@@H](C(=O)NCCc1ccccc1)C(C)C(N)=O] | Compound LXXIV |
| 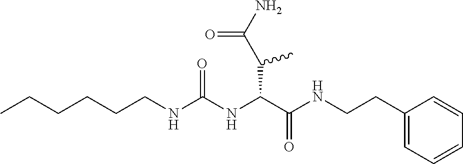 [SMILES: CCCCCCNC(=O)N[C@@H](C(=O)NCCc1ccccc1)C(C)C(N)=O] | Compound LXXV |
| 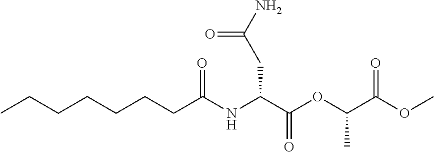 [SMILES: CCCCCCCC(=O)N[C@H](CC(N)=O)C(=O)O[C@@H](C)C(=O)OC] | Compound LXXVI |
| 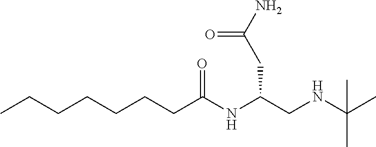 [SMILES: CCCCCCCC(=O)N[C@@H](CNC(C)(C)C)CC(N)=O] | Compound LXXVII |
| 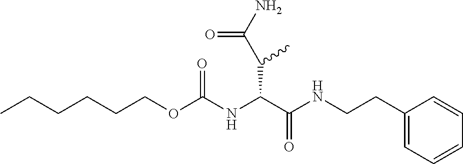 [SMILES: CCCCCCOC(=O)N[C@@H](C(=O)NCCc1ccccc1)C(C)C(N)=O] | Compound LXXVIII |
| 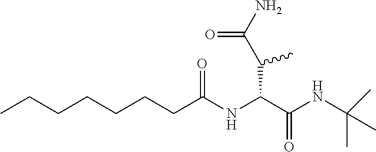 [SMILES: CCCCCCCC(=O)N[C@@H](C(=O)NC(C)(C)C)C(C)C(N)=O] | Compound LXXIX |
| 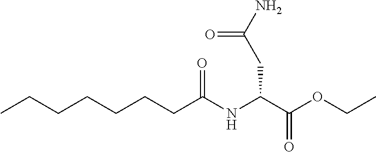 [SMILES: CCCCCCCC(=O)N[C@H](CC(N)=O)C(=O)OCC] | Compound LXXX |

In some embodiments, the present disclosure is drawn to one or more compounds recited in Table 2.

TABLE 2

| | |
|---|---|
| 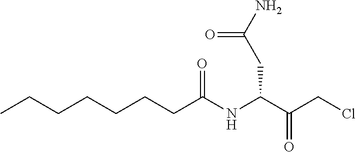 [SMILES: CCCCCCCC(=O)N[C@H](CC(N)=O)C(=O)CCl] | Compound LXXXI |
| 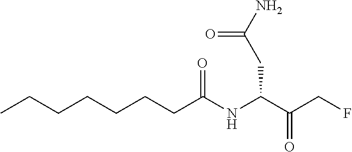 [SMILES: CCCCCCCC(=O)N[C@H](CC(N)=O)C(=O)CF] | Compound LXXXII |
| 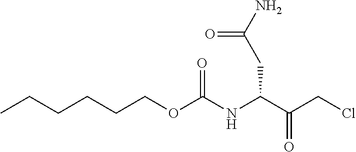 [SMILES: CCCCCCOC(=O)N[C@H](CC(N)=O)C(=O)CCl] | Compound LXXXIII |
| 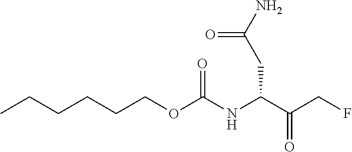 [SMILES: CCCCCCOC(=O)N[C@H](CC(N)=O)C(=O)CF] | Compound LXXXIV |
| 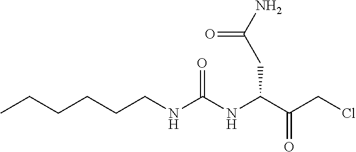 [SMILES: CCCCCCNC(=O)N[C@H](CC(N)=O)C(=O)CCl] | Compound LXXXV |
| 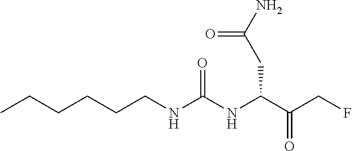 [SMILES: CCCCCCNC(=O)N[C@H](CC(N)=O)C(=O)CF] | Compound LXXXVI |
| 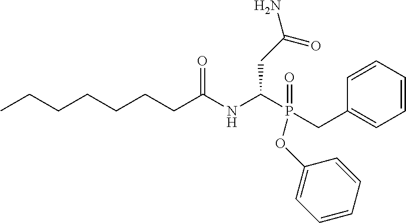 [SMILES: CCCCCCCC(=O)N[C@H](CC(N)=O)P(=O)(Cc1ccccc1)Oc1ccccc1] | Compound LXXXVII |
| 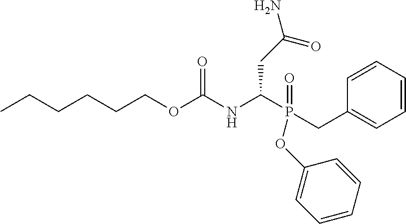 [SMILES: CCCCCCOC(=O)N[C@H](CC(N)=O)P(=O)(Cc1ccccc1)Oc1ccccc1] | Compound LXXXVIII |
| 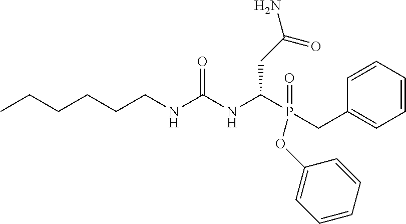 [SMILES: CCCCCCNC(=O)N[C@H](CC(N)=O)P(=O)(Cc1ccccc1)Oc1ccccc1] | Compound LXXXIX |
| 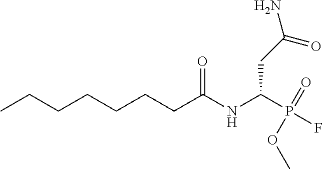 [SMILES: CCCCCCCC(=O)N[C@H](CC(N)=O)P(=O)(F)OC] | Compound XC |
| 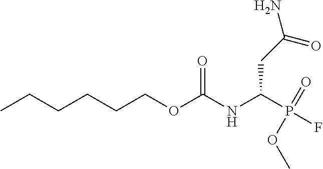 [SMILES: CCCCCCOC(=O)N[C@H](CC(N)=O)P(=O)(F)OC] | Compound XCI |
| 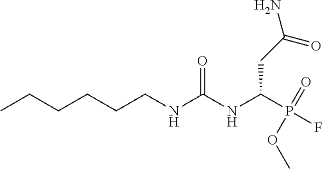 [SMILES: CCCCCCNC(=O)N[C@H](CC(N)=O)P(=O)(F)OC] | Compound XCII |
| 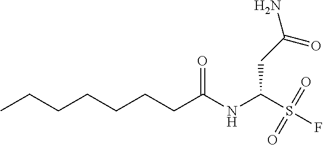 [SMILES: CCCCCCCC(=O)N[C@H](CC(N)=O)S(=O)(=O)F] | Compound XCIII |
| 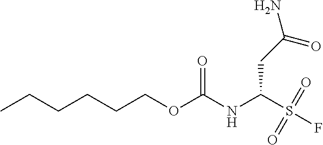 [SMILES: CCCCCCOC(=O)N[C@H](CC(N)=O)S(=O)(=O)F] | Compound XCIV |
| 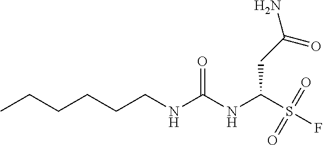 [SMILES: CCCCCCNC(=O)N[C@H](CC(N)=O)S(=O)(=O)F] | Compound XCV |
| 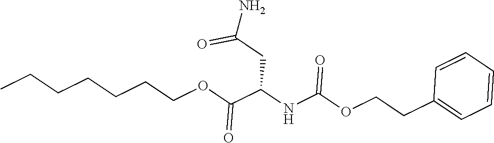 [SMILES: CCCCCCCOC(=O)[C@H](CC(N)=O)NC(=O)OCCc1ccccc1] | Compound XCVI |
| 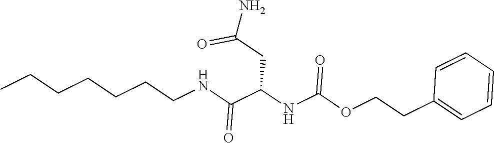 [SMILES: CCCCCCCNC(=O)[C@H](CC(N)=O)NC(=O)OCCc1ccccc1] | Compound XCVII |
| 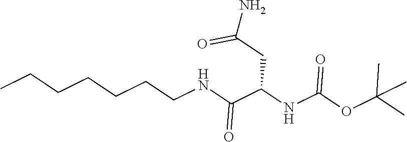 [SMILES: CCCCCCCNC(=O)[C@H](CC(N)=O)NC(=O)OC(C)(C)C] | Compound XCVIII |
| 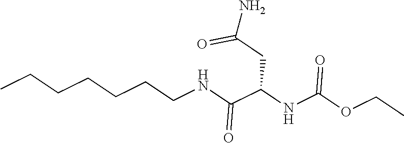 [SMILES: CCCCCCCNC(=O)[C@H](CC(N)=O)NC(=O)OCC] | Compound XCIX |
| 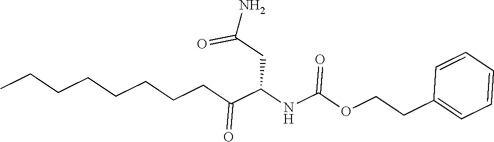 [SMILES: CCCCCCCCC(=O)[C@H](CC(N)=O)NC(=O)OCCc1ccccc1] | Compound C |
| 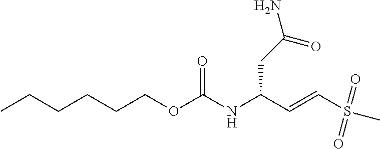 [SMILES: CCCCCCOC(=O)N[C@@H](/C=C/S(C)(=O)=O)CC(N)=O] | Compound CI |
| 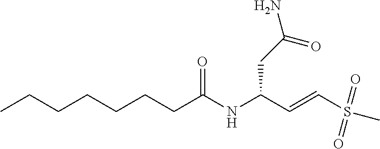 [SMILES: CCCCCCCC(=O)N[C@@H](/C=C/S(C)(=O)=O)CC(N)=O] | Compound CII |
| 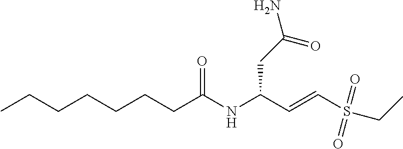 [SMILES: CCCCCCCC(=O)N[C@@H](/C=C/S(=O)(=O)CC)CC(N)=O] | Compound CIII |
| 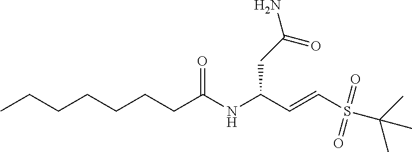 [SMILES: CCCCCCCC(=O)N[C@@H](/C=C/S(=O)(=O)C(C)(C)C)CC(N)=O] | Compound CIV |
| 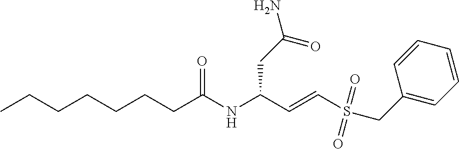 [SMILES: CCCCCCCC(=O)N[C@@H](/C=C/S(=O)(=O)Cc1ccccc1)CC(N)=O] | Compound CV |
| 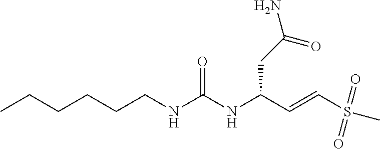 [SMILES: CCCCCCNC(=O)N[C@@H](/C=C/S(C)(=O)=O)CC(N)=O] | Compound CVI |
| 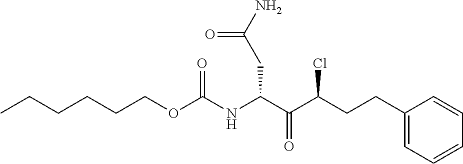 [SMILES: CCCCCCOC(=O)N[C@H](CC(N)=O)C(=O)[C@@H](Cl)CCc1ccccc1] | Compound CVII |
| 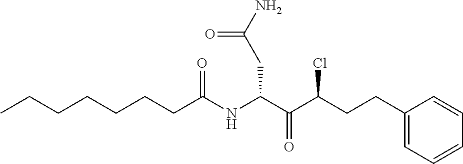 [SMILES: CCCCCCCC(=O)N[C@H](CC(N)=O)C(=O)[C@@H](Cl)CCc1ccccc1] | Compound CVIII |
| 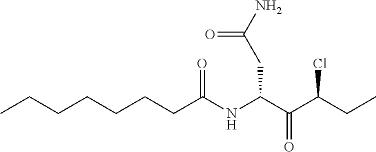 [SMILES: CCCCCCCC(=O)N[C@H](CC(N)=O)C(=O)[C@@H](Cl)CC] | Compound CIX |
| 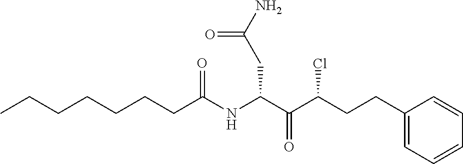 [SMILES: CCCCCCCC(=O)N[C@H](CC(N)=O)C(=O)[C@H](Cl)CCc1ccccc1] | Compound CX |
| 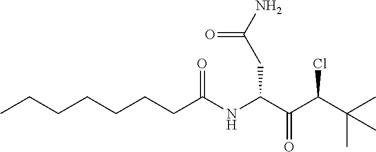 [SMILES: CCCCCCCC(=O)N[C@H](CC(N)=O)C(=O)[C@@H](Cl)C(C)(C)C] | Compound CXI |
| 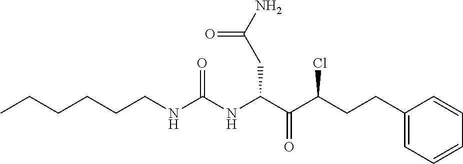 [SMILES: CCCCCCNC(=O)N[C@H](CC(N)=O)C(=O)[C@@H](Cl)CCc1ccccc1] | Compound CXII |
| 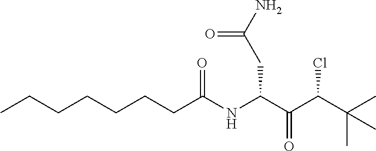 [SMILES: CCCCCCCC(=O)N[C@H](CC(N)=O)C(=O)[C@H](Cl)C(C)(C)C] | Compound CXIII |
| 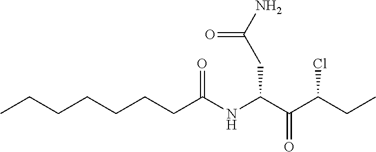 [SMILES: CCCCCCCC(=O)N[C@H](CC(N)=O)C(=O)[C@H](Cl)CC] | Compound CXIV |
| 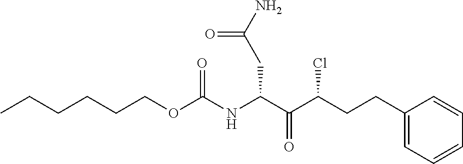 [SMILES: CCCCCCOC(=O)N[C@H](CC(N)=O)C(=O)[C@H](Cl)CCc1ccccc1] | Compound CXV |
| 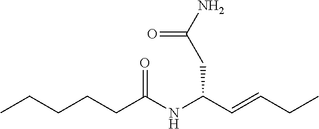 [SMILES: CC/C=C/[C@@H](CC(N)=O)NC(=O)CCCCC] | Compound CXVI |
| 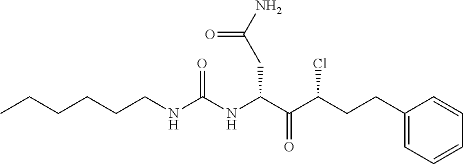 [SMILES: CCCCCCNC(=O)N[C@H](CC(N)=O)C(=O)[C@H](Cl)CCc1ccccc1] | Compound CXVII |
| 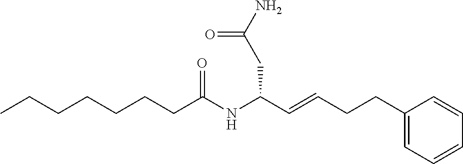 [SMILES: CCCCCCCC(=O)N[C@@H](/C=C/CCc1ccccc1)CC(N)=O] | Compound CXVIII |
| 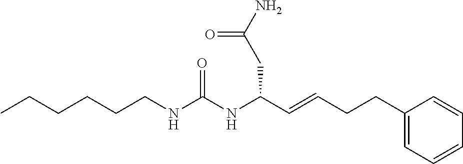 [SMILES: CCCCCCNC(=O)N[C@@H](/C=C/CCc1ccccc1)CC(N)=O] | Compound CXIX |
| 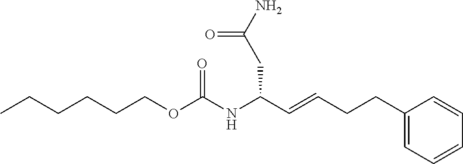 [SMILES: CCCCCCOC(=O)N[C@@H](/C=C/CCc1ccccc1)CC(N)=O] | Compound CXX |
| 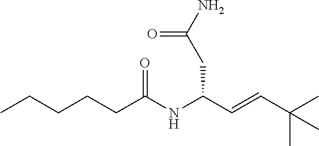 [SMILES: CCCCCC(=O)N[C@@H](/C=C/C(C)(C)C)CC(N)=O] | Compound CXXI |
| 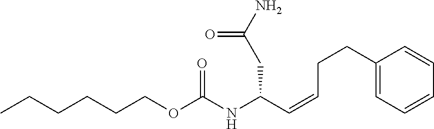 [SMILES: CCCCCCOC(=O)N[C@@H](/C=C\CCc1ccccc1)CC(N)=O] | Compound CXXII |
| 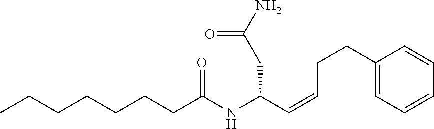 [SMILES: CCCCCCCC(=O)N[C@@H](/C=C\CCc1ccccc1)CC(N)=O] | Compound CXXIII |
| 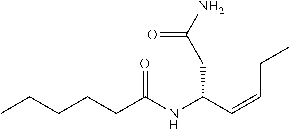 [SMILES: CC/C=C\[C@@H](CC(N)=O)NC(=O)CCCCC] | Compound CXXIV |
| 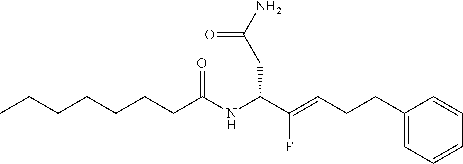 [SMILES: CCCCCCCC(=O)N[C@H](CC(N)=O)/C(F)=C/CCc1ccccc1] | Compound CXXV |
| 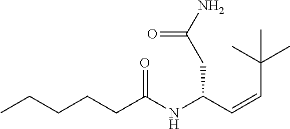 [SMILES: CCCCCC(=O)N[C@@H](/C=C\C(C)(C)C)CC(N)=O] | Compound CXXVI |
| 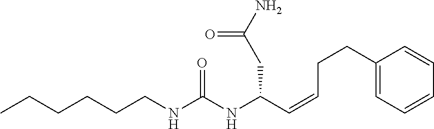 [SMILES: CCCCCCNC(=O)N[C@@H](/C=C\CCc1ccccc1)CC(N)=O] | Compound CXXVII |
| 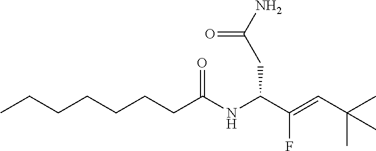 [SMILES: CCCCCCCC(=O)N[C@H](CC(N)=O)/C(F)=C/C(C)(C)C] | Compound CXXVIII |
| 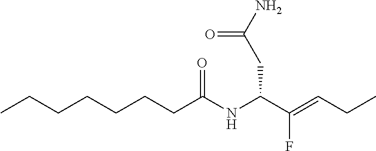 [SMILES: CC/C=C(\F)[C@@H](CC(N)=O)NC(=O)CCCCCCC] | Compound CXXIX |
| 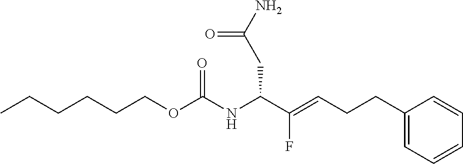 [SMILES: CCCCCCOC(=O)N[C@H](CC(N)=O)/C(F)=C/CCc1ccccc1] | Compound CXXX |
| 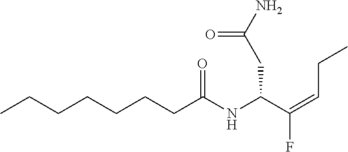 [SMILES: CC/C=C(/F)[C@@H](CC(N)=O)NC(=O)CCCCCCC] | Compound CXXXI |
| 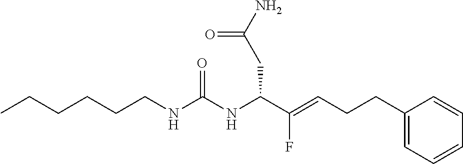 [SMILES: CCCCCCNC(=O)N[C@H](CC(N)=O)/C(F)=C/CCc1ccccc1] | Compound CXXXII |
| 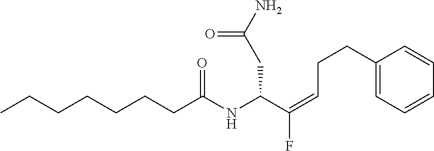 [SMILES: CCCCCCCC(=O)N[C@H](CC(N)=O)/C(F)=C\CCc1ccccc1] | Compound CXXXIII |
| 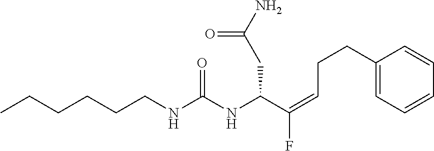 [SMILES: CCCCCCNC(=O)N[C@H](CC(N)=O)/C(F)=C\CCc1ccccc1] | Compound CXXXIV |
| 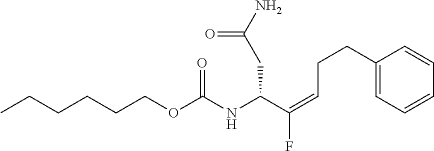 [SMILES: CCCCCCOC(=O)N[C@H](CC(N)=O)/C(F)=C\CCc1ccccc1] | Compound CXXXV |
| 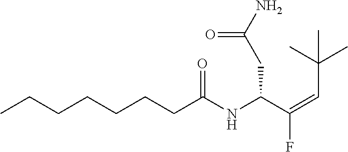 [SMILES: CCCCCCCC(=O)N[C@H](CC(N)=O)/C(F)=C\C(C)(C)C] | Compound CXXXVI |
| 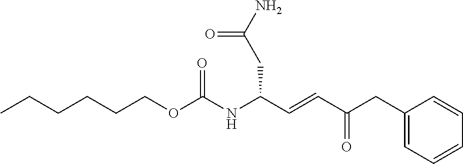 [SMILES: CCCCCCOC(=O)N[C@@H](/C=C/C(=O)Cc1ccccc1)CC(N)=O] | Compound CXXXVII |
| 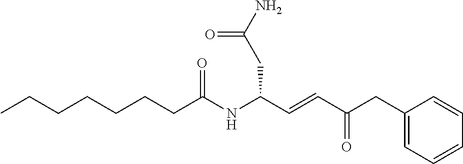 [SMILES: CCCCCCCC(=O)N[C@@H](/C=C/C(=O)Cc1ccccc1)CC(N)=O] | Compound CXXXVIII |
| 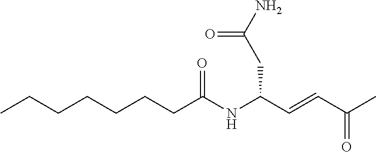 [SMILES: CCCCCCCC(=O)N[C@@H](/C=C/C(C)=O)CC(N)=O] | Compound CXXXIX |
| 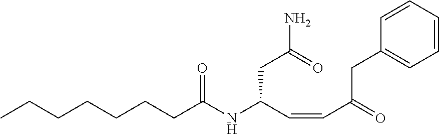 [SMILES: CCCCCCCC(=O)N[C@@H](/C=C\C(=O)Cc1ccccc1)CC(N)=O] | Compound CXL |
| 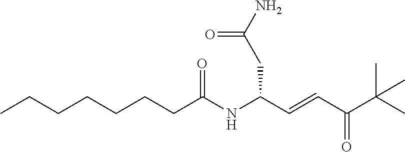 [SMILES: CCCCCCCC(=O)N[C@@H](/C=C/C(=O)C(C)(C)C)CC(N)=O] | Compound CXLI |
| 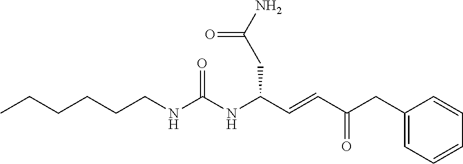 [SMILES: CCCCCCNC(=O)N[C@@H](/C=C/C(=O)Cc1ccccc1)CC(N)=O] | Compound CXLII |
| 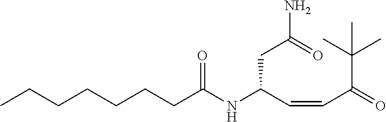 [SMILES: CCCCCCCC(=O)N[C@@H](/C=C\C(=O)C(C)(C)C)CC(N)=O] | Compound CXLIII |
| 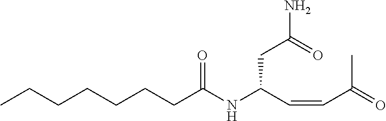 [SMILES: CCCCCCCC(=O)N[C@@H](/C=C\C(C)=O)CC(N)=O] | Compound CXLIV |
| 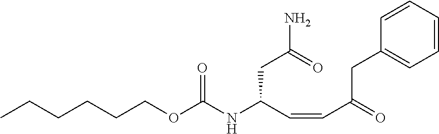 [SMILES: CCCCCCOC(=O)N[C@@H](/C=C\C(=O)Cc1ccccc1)CC(N)=O] | Compound CXLV |
| 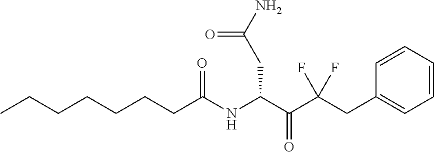 [SMILES: CCCCCCCC(=O)N[C@H](CC(N)=O)C(=O)C(F)(F)Cc1ccccc1] | Compound CXLVI |
| 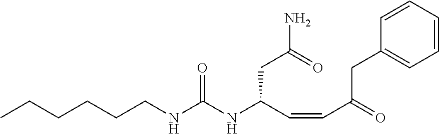 [SMILES: CCCCCCNC(=O)N[C@@H](/C=C\C(=O)Cc1ccccc1)CC(N)=O] | Compound CXLVII |

In some embodiments, the present disclosure is drawn to one or more compounds recited in Table 3.

TABLE 3

| | |
|---|---|
| 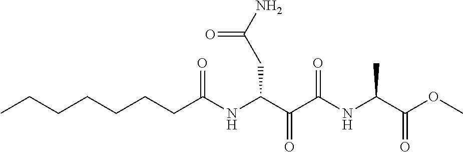 [SMILES: CCCCCCCC(=O)N[C@H](CC(N)=O)C(=O)C(=O)N[C@@H](C)C(=O)OC] | Compound CXLVIII |
| 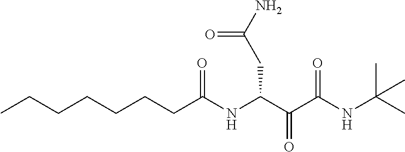 [SMILES: CCCCCCCC(=O)N[C@H](CC(N)=O)C(=O)C(=O)NC(C)(C)C] | Compound CXLIX |
| 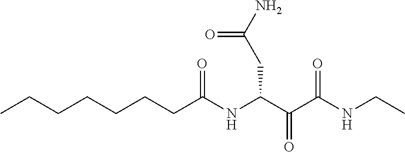 [SMILES: CCCCCCCC(=O)N[C@H](CC(N)=O)C(=O)C(=O)NCC] | Compound CL |
| 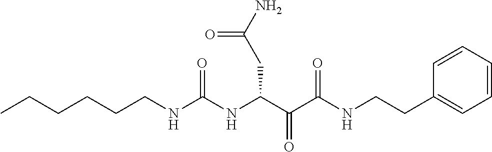 [SMILES: CCCCCCNC(=O)N[C@H](CC(N)=O)C(=O)C(=O)NCCc1ccccc1] | Compound CLI |
| 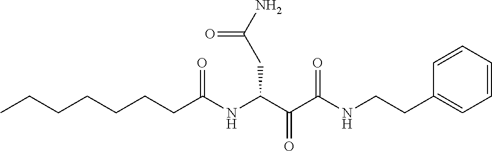 [SMILES: CCCCCCCC(=O)N[C@H](CC(N)=O)C(=O)C(=O)NCCc1ccccc1] | Compound CLII |
| 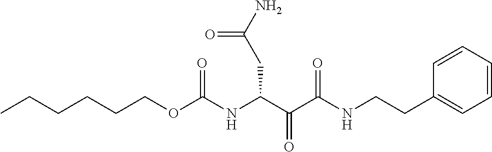 [SMILES: CCCCCCOC(=O)N[C@H](CC(N)=O)C(=O)C(=O)NCCc1ccccc1] | Compound CLIII |
| 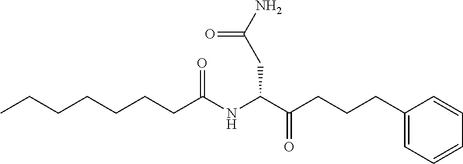 [SMILES: CCCCCCCC(=O)N[C@H](CC(N)=O)C(=O)CCCc1ccccc1] | Compound CLIV |
| 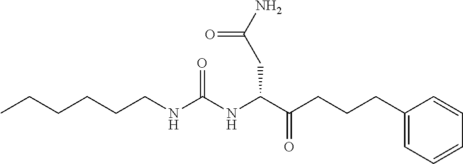 [SMILES: CCCCCCNC(=O)N[C@H](CC(N)=O)C(=O)CCCc1ccccc1] | Compound CLV |
| 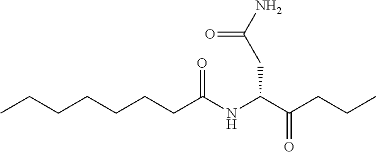 [SMILES: CCCCCCCC(=O)N[C@H](CC(N)=O)C(=O)CCC] | Compound CLVI |
| 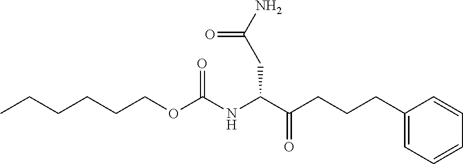 [SMILES: CCCCCCOC(=O)N[C@H](CC(N)=O)C(=O)CCCc1ccccc1] | Compound CLVII |
| 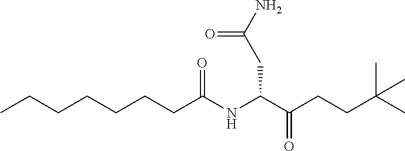 [SMILES: CCCCCCCC(=O)N[C@H](CC(N)=O)C(=O)CCC(C)(C)C] | Compound CLVIII |
| 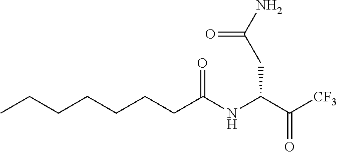 [SMILES: CCCCCCCC(=O)N[C@H](CC(N)=O)C(=O)C(F)(F)F] | Compound CLIX |
| 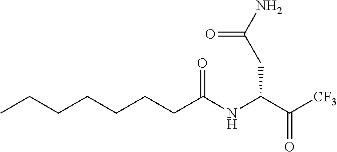 [SMILES: CCCCCCCC(=O)N[C@H](CC(N)=O)C(=O)C(F)(F)F] | Compound CLX |
| 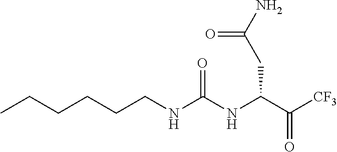 [SMILES: CCCCCCNC(=O)N[C@H](CC(N)=O)C(=O)C(F)(F)F] | Compound CLXI |
| 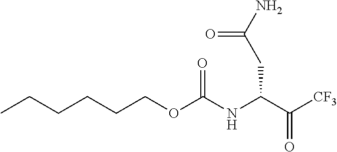 [SMILES: CCCCCCOC(=O)N[C@H](CC(N)=O)C(=O)C(F)(F)F] | Compound CLXII |
| 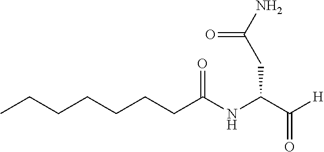 [SMILES: CCCCCCCC(=O)N[C@@H](C=O)CC(N)=O] | Compound CLXIII |
| 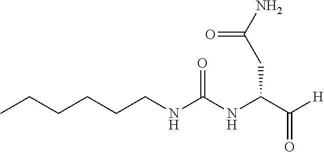 [SMILES: CCCCCCNC(=O)N[C@@H](C=O)CC(N)=O] | Compound CLXIV |
| 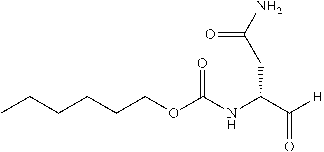 [SMILES: CCCCCCOC(=O)N[C@@H](C=O)CC(N)=O] | Compound CLXV |
| 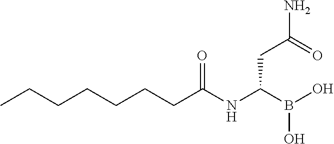 [SMILES: CCCCCCCC(=O)N[C@H](CC(N)=O)B(O)O] | Compound CLXVI |
| 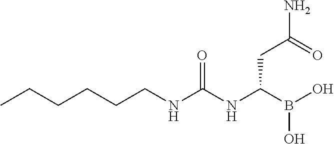 [SMILES: CCCCCCNC(=O)N[C@H](CC(N)=O)B(O)O] | Compound CLXVII |
| 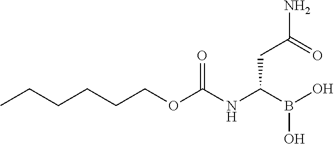 [SMILES: CCCCCCOC(=O)N[C@H](CC(N)=O)B(O)O] | Compound CLXVIII |
| 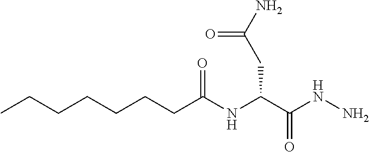 [SMILES: CCCCCCCC(=O)N[C@H](CC(N)=O)C(=O)NN] | Compound CLXIX |
| 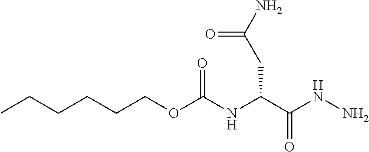 [SMILES: CCCCCCOC(=O)N[C@H](CC(N)=O)C(=O)NN] | Compound CLXX |
| 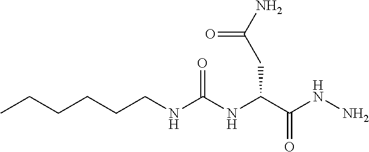 [SMILES: CCCCCCNC(=O)N[C@H](CC(N)=O)C(=O)NN] | Compound CLXXI |
| 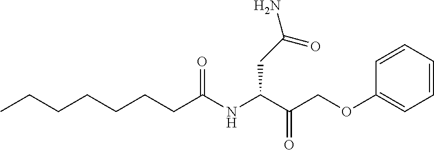 [SMILES: CCCCCCCC(=O)N[C@H](CC(N)=O)C(=O)COc1ccccc1] | Compound CLXXII |
| 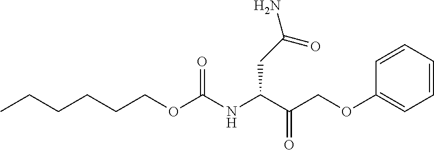 [SMILES: CCCCCCOC(=O)N[C@H](CC(N)=O)C(=O)COc1ccccc1] | Compound CLXXIII |
| 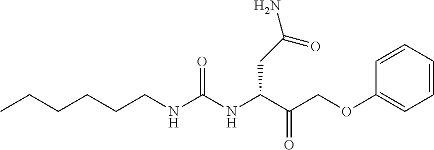 [SMILES: CCCCCCNC(=O)N[C@H](CC(N)=O)C(=O)COc1ccccc1] | Compound CLXXIV |
| 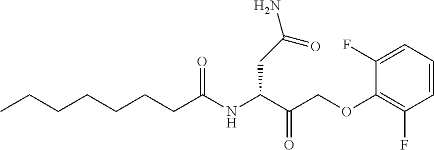 [SMILES: CCCCCCCC(=O)N[C@H](CC(N)=O)C(=O)COc1c(F)cccc1F] | Compound CLXXV |
| 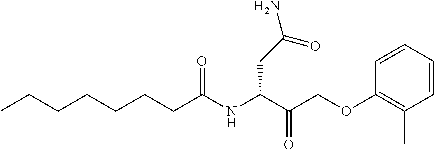 [SMILES: CCCCCCCC(=O)N[C@H](CC(N)=O)C(=O)COc1ccccc1C] | Compound CLXXVI |
| 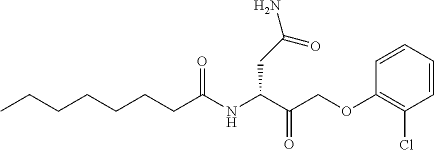 [SMILES: CCCCCCCC(=O)N[C@H](CC(N)=O)C(=O)COc1ccccc1Cl] | Compound CLXXVII |
| 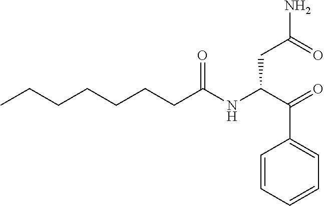 [SMILES: CCCCCCCC(=O)N[C@H](CC(N)=O)C(=O)c1ccccc1] | Compound CLXXVIII |
| 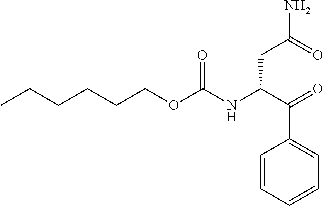 [SMILES: CCCCCCOC(=O)N[C@H](CC(N)=O)C(=O)c1ccccc1] | Compound CLXXIX |
| 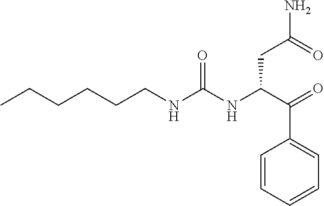 [SMILES: CCCCCCNC(=O)N[C@H](CC(N)=O)C(=O)c1ccccc1] | Compound CLXXX |
| 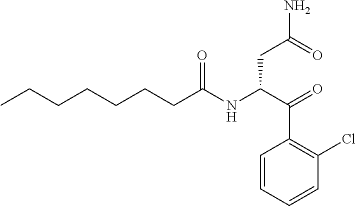 [SMILES: CCCCCCCC(=O)N[C@H](CC(N)=O)C(=O)c1ccccc1Cl] | Compound CLXXXI |
| 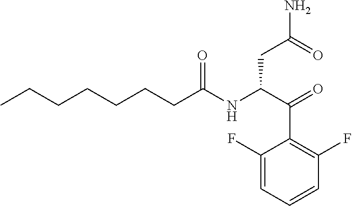 [SMILES: CCCCCCCC(=O)N[C@H](CC(N)=O)C(=O)c1c(F)cccc1F] | Compound CLXXXII |
| 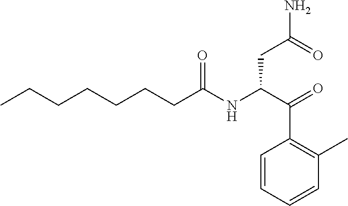 [SMILES: CCCCCCCC(=O)N[C@H](CC(N)=O)C(=O)c1ccccc1C] | Compound CLXXXIII |
| 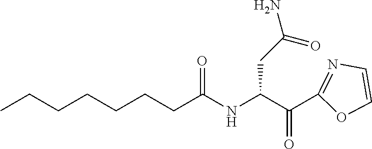 [SMILES: CCCCCCCC(=O)N[C@H](CC(N)=O)C(=O)c1ncco1] | Compound CLXXXIV |
| 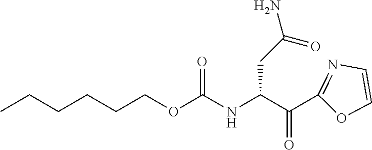 [SMILES: CCCCCCOC(=O)N[C@H](CC(N)=O)C(=O)c1ncco1] | Compound CLXXXV |
| 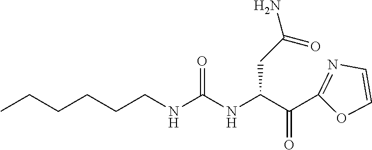 [SMILES: CCCCCCNC(=O)N[C@H](CC(N)=O)C(=O)c1ncco1] | Compound CLXXXVI |
| 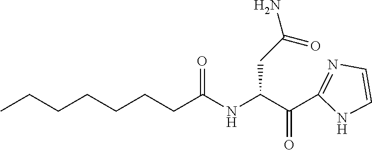 [SMILES: CCCCCCCC(=O)N[C@H](CC(N)=O)C(=O)c1ncc[nH]1] | Compound CLXXXVII |
| 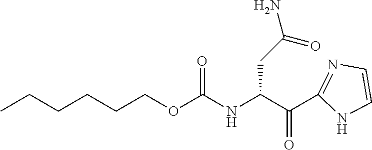 [SMILES: CCCCCCOC(=O)N[C@H](CC(N)=O)C(=O)c1ncc[nH]1] | Compound CLXXXVIII |
| 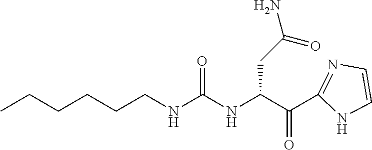 [SMILES: CCCCCCNC(=O)N[C@H](CC(N)=O)C(=O)c1ncc[nH]1] | Compound CLXXXIX |
| 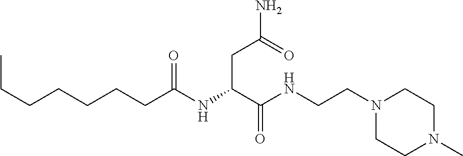 [SMILES: CCCCCCCC(=O)N[C@H](CC(N)=O)C(=O)NCCN1CCN(C)CC1] | Compound CXC |
| 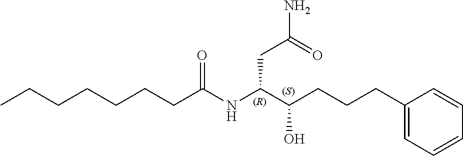 | Compound CXCI |
| 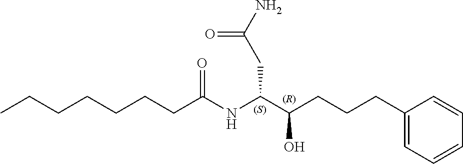 | Compound CXCII |

In some embodiments, the present disclosure is drawn to one or more compounds recited in Table 4.

TABLE 4

| | |
|---|---|
| 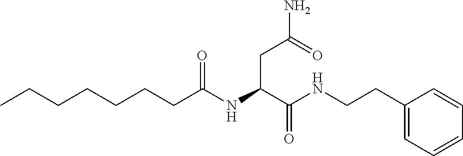 [SMILES: CCCCCCCC(=O)N[C@@H](CC(N)=O)C(=O)NCCc1ccccc1] | Compound CXCIII |
| 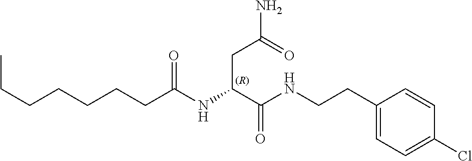 | Compound CXCIV |
| 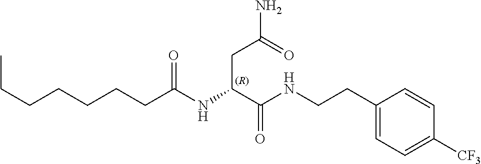 | Compound CXCV |
| 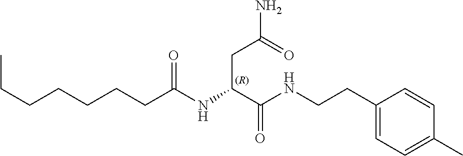 | Compound CXCVI |
| 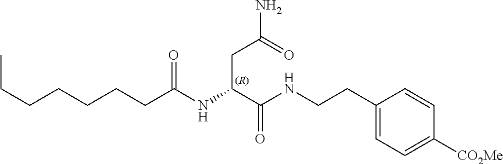 | Compound CXCVII |
| 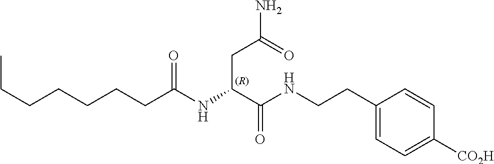 | Compound CXCVIII |
| 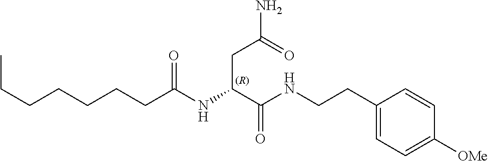 | Compound CXCIX |
| 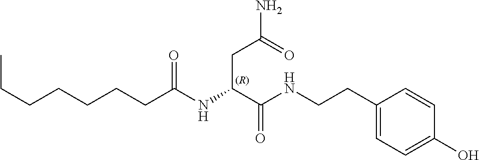 | Compound CC |
| 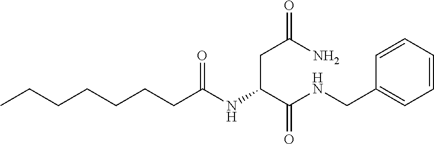 [SMILES: CCCCCCCC(=O)N[C@H](CC(N)=O)C(=O)NCc1ccccc1] | Compound CCI |
| 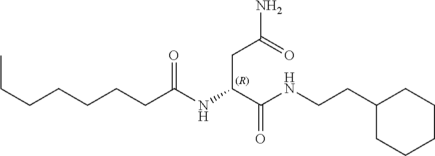 | Compound CCII |
| 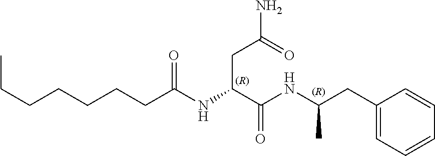 | Compound CCIII |
| 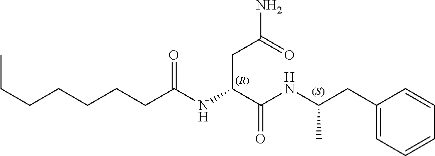 | Compound CCIV |
| 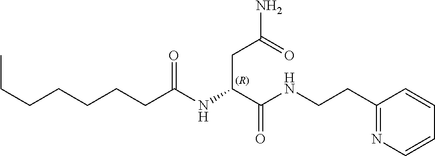 | Compound CCV |
| 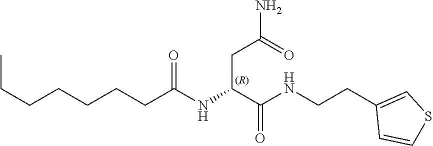 | Compound CCVI |
| 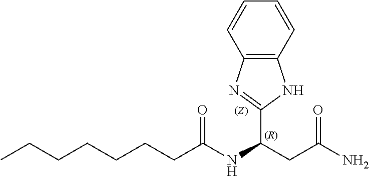 | Compound CCVII |
| 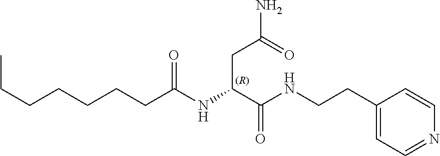 | Compound CCVIII |
| 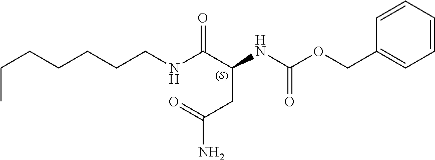 | Compound CCIX |
| 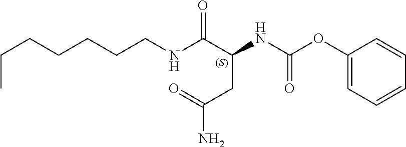 | Compound CCX |
| 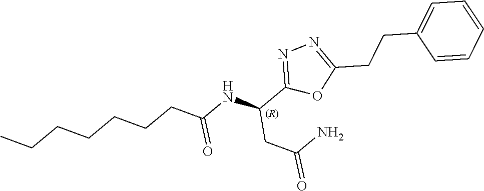 | Compound CCXI |
| 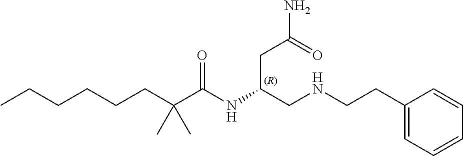 | Compound CCXII |
| 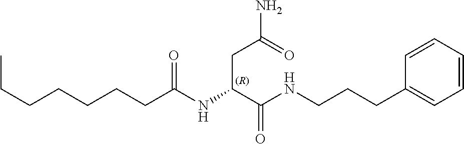 | Compound CCXIII |
| 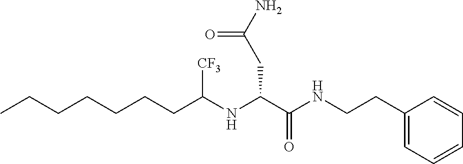 [SMILES: CCCCCCCC(N[C@H](CC(N)=O)C(=O)NCCc1ccccc1)C(F)(F)F] | Compound CCXIV |
| 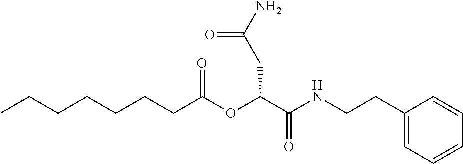 [SMILES: CCCCCCCC(=O)O[C@H](CC(N)=O)C(=O)NCCc1ccccc1] | Compound CCXV |
| 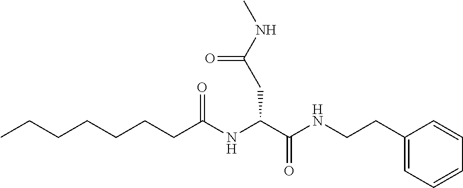 [SMILES: CCCCCCCC(=O)N[C@H](CC(=O)NC)C(=O)NCCc1ccccc1] | Compound CCXVI |
| 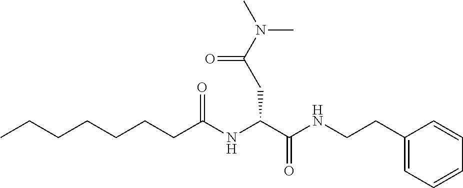 [SMILES: CCCCCCCC(=O)N[C@H](CC(=O)N(C)C)C(=O)NCCc1ccccc1] | Compound CCXVII |
| 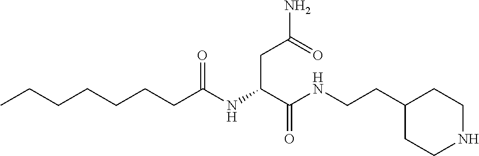 [SMILES: CCCCCCCC(=O)N[C@H](CC(N)=O)C(=O)NCCC1CCNCC1] | Compound CCXVIII |
| 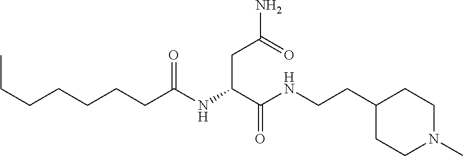 [SMILES: CCCCCCCC(=O)N[C@H](CC(N)=O)C(=O)NCCC1CCN(C)CC1] | Compound CCXIX |
| 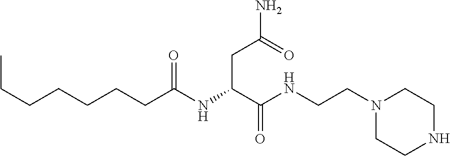 [SMILES: CCCCCCCC(=O)N[C@H](CC(N)=O)C(=O)NCCN1CCNCC1] | Compound CCXX |
| 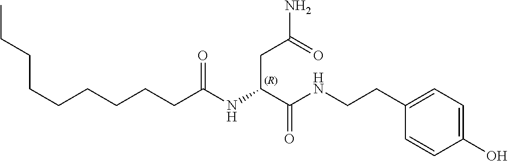 | Compound CCXXI |
| 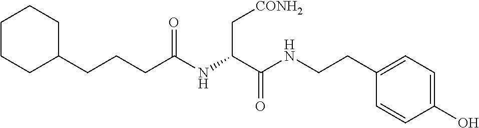 [SMILES: NC(=O)C[C@@H](NC(=O)CCCC1CCCCC1)C(=O)NCCc1ccc(O)cc1] | Compound CCXXII |
| 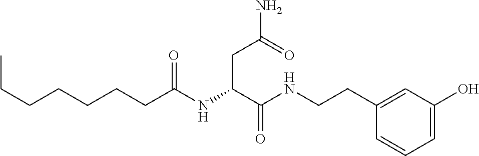 [SMILES: CCCCCCCC(=O)N[C@H](CC(N)=O)C(=O)NCCc1cccc(O)c1] | Compound CCXXIII |
| 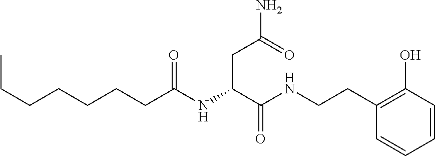 [SMILES: CCCCCCCC(=O)N[C@H](CC(N)=O)C(=O)NCCc1ccccc1O] | Compound CCXXIV |
| 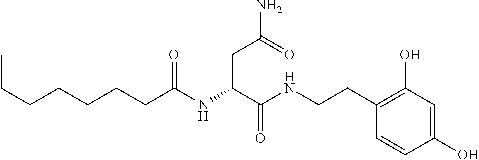 [SMILES: CCCCCCCC(=O)N[C@H](CC(N)=O)C(=O)NCCc1ccc(O)cc1O] | Compound CCXXV |
| 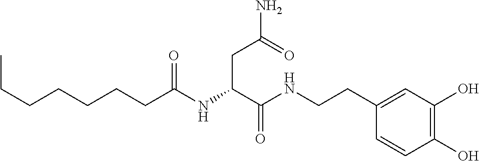 [SMILES: CCCCCCCC(=O)N[C@H](CC(N)=O)C(=O)NCCc1ccc(O)c(O)c1] | Compound CCXXVI |
| 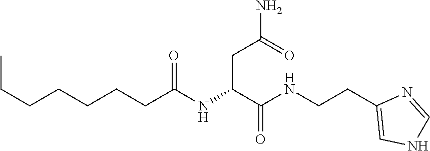 [SMILES: CCCCCCCC(=O)N[C@H](CC(N)=O)C(=O)NCCc1c[nH]cn1] | Compound CCXXVII |
| 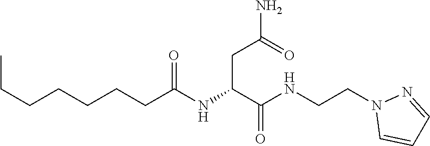 [SMILES: CCCCCCCC(=O)N[C@H](CC(N)=O)C(=O)NCCn1cccn1] | Compound CCXXVIII |
| 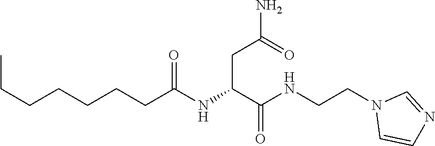 [SMILES: CCCCCCCC(=O)N[C@H](CC(N)=O)C(=O)NCCn1ccnc1] | Compound CCXXIX |
| 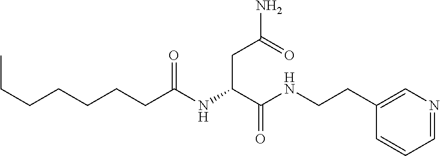 [SMILES: CCCCCCCC(=O)N[C@H](CC(N)=O)C(=O)NCCc1cccnc1] | Compound CCXXX |
| 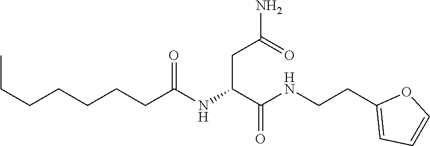 [SMILES: CCCCCCCC(=O)N[C@H](CC(N)=O)C(=O)NCCc1ccco1] | Compound CCXXXI |
| 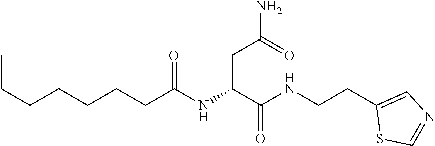 [SMILES: CCCCCCCC(=O)N[C@H](CC(N)=O)C(=O)NCCc1cncs1] | Compound CCXXXII |
| 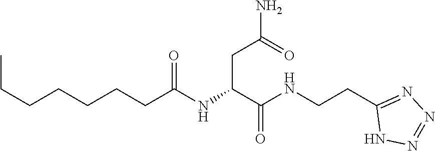 [SMILES: CCCCCCCC(=O)N[C@H](CC(N)=O)C(=O)NCCc1nnn[nH]1] | Compound CCXXXIII |
| 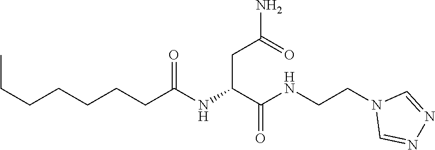 [SMILES: CCCCCCCC(=O)N[C@H](CC(N)=O)C(=O)NCCn1cnnc1] | Compound CCXXXIV |
| 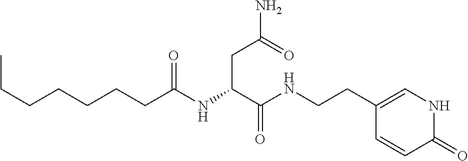 [SMILES: CCCCCCCC(=O)N[C@H](CC(N)=O)C(=O)NCCc1ccc(=O)[nH]c1] | Compound CCXXXV |
| 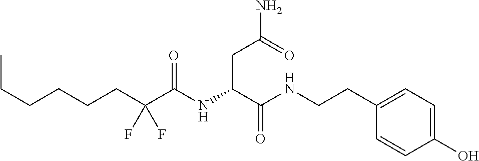 [SMILES: CCCCCCC(F)(F)C(=O)N[C@H](CC(N)=O)C(=O)NCCc1ccc(O)cc1] | Compound CCXXXVI |
| 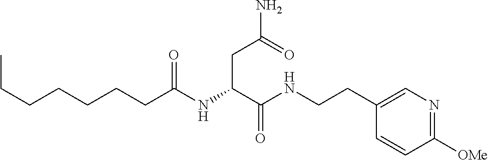 [SMILES: CCCCCCCC(=O)N[C@H](CC(N)=O)C(=O)NCCc1ccc(OC)nc1] | Compound CCXXXVII |
| 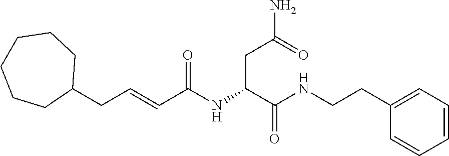 [SMILES: NC(=O)C[C@@H](NC(=O)/C=C/CC1CCCCCC1)C(=O)NCCc1ccccc1] | Compound CCXXXVIII |
| 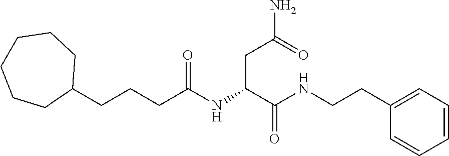 [SMILES: NC(=O)C[C@@H](NC(=O)CCCC1CCCCCC1)C(=O)NCCc1ccccc1] | Compound CCXXXIX |
| 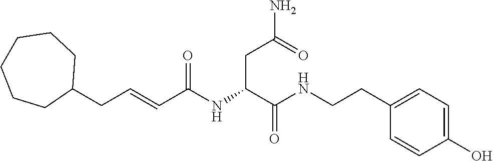 [SMILES: NC(=O)C[C@@H](NC(=O)/C=C/CC1CCCCCC1)C(=O)NCCc1ccc(O)cc1] | Compound CCXL |
| 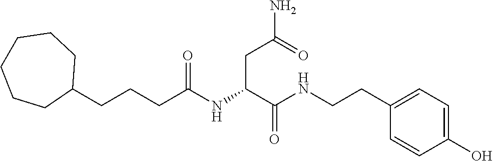 [SMILES: NC(=O)C[C@@H](NC(=O)CCCC1CCCCCC1)C(=O)NCCc1ccc(O)cc1] | Compound CCXLI |
| 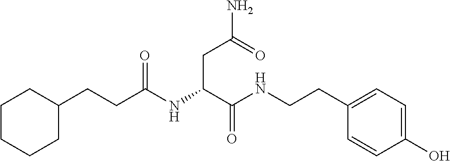 [SMILES: NC(=O)C[C@@H](NC(=O)CCC1CCCCC1)C(=O)NCCc1ccc(O)cc1] | Compound CCXLII |
| 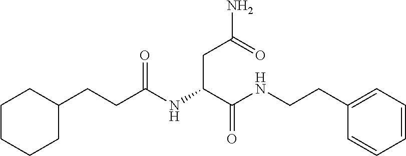 [SMILES: NC(=O)C[C@@H](NC(=O)CCC1CCCCC1)C(=O)NCCc1ccccc1] | Compound CCXLIII |
| 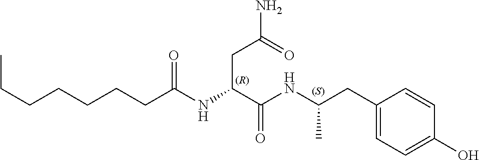 | Compound CCXLIV |
| 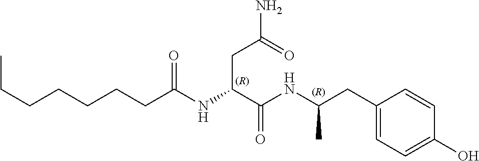 | Compound CCXLV |
| 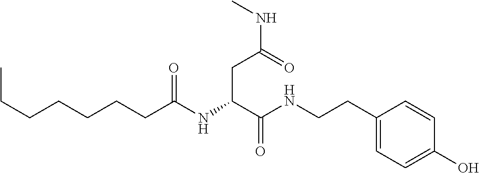 [SMILES: CCCCCCCC(=O)N[C@H](CC(=O)NC)C(=O)NCCc1ccc(O)cc1] | Compound CCXLVI |
| 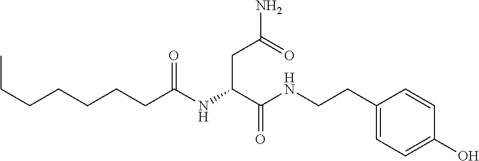 [SMILES: CCCCCCCC(=O)N[C@H](CC(N)=O)C(=O)NCCc1ccc(O)cc1] | Compound CCXLVII |
| 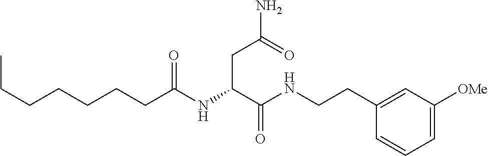 [SMILES: CCCCCCCC(=O)N[C@H](CC(N)=O)C(=O)NCCc1cccc(OC)c1] | Compound CCXLVIII |
| 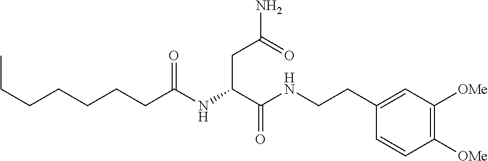 [SMILES: CCCCCCCC(=O)N[C@H](CC(N)=O)C(=O)NCCc1ccc(OC)c(OC)c1] | Compound CCXLIX |
| 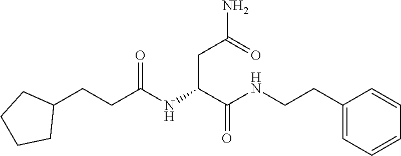 [SMILES: NC(=O)C[C@@H](NC(=O)CCC1CCCC1)C(=O)NCCc1ccccc1] | Compound CCL |
| 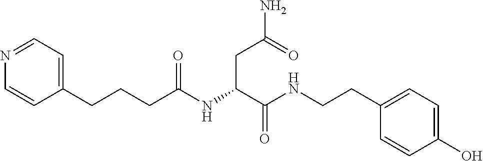 [SMILES: NC(=O)C[C@@H](NC(=O)CCCc1ccncc1)C(=O)NCCc1ccc(O)cc1] | Compound CCLI |
| 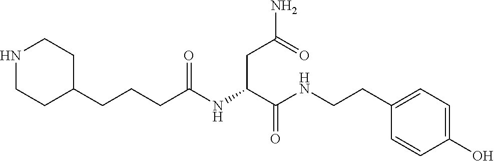 [SMILES: NC(=O)C[C@@H](NC(=O)CCCC1CCNCC1)C(=O)NCCc1ccc(O)cc1] | Compound CCLII |
| 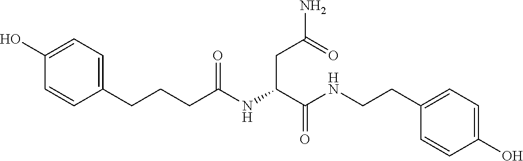 [SMILES: NC(=O)C[C@@H](NC(=O)CCCc1ccc(O)cc1)C(=O)NCCc1ccc(O)cc1] | Compound CCLIII |
| 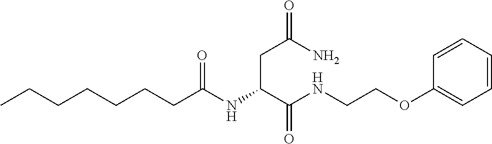 [SMILES: CCCCCCCC(=O)N[C@H](CC(N)=O)C(=O)NCCOc1ccccc1] | Compound CCLIV |
| 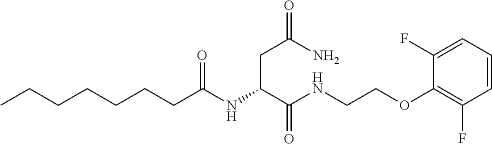 [SMILES: CCCCCCCC(=O)N[C@H](CC(N)=O)C(=O)NCCOc1c(F)cccc1F] | Compound CCLV |
| 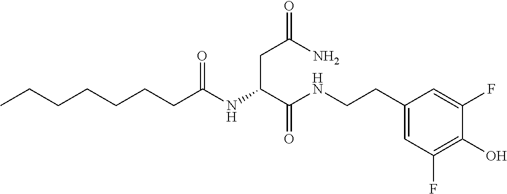 [SMILES: CCCCCCCC(=O)N[C@H](CC(N)=O)C(=O)NCCc1cc(F)c(O)c(F)c1] | Compound CCLVI |
| 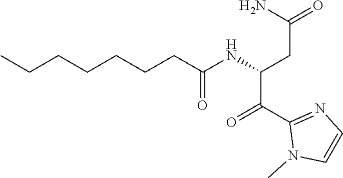 [SMILES: CCCCCCCC(=O)N[C@H](CC(N)=O)C(=O)c1nccn1C] | Compound CCLVII |
| 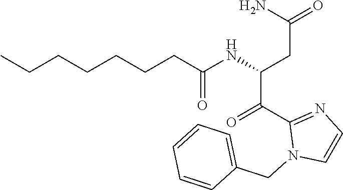 [SMILES: CCCCCCCC(=O)N[C@H](CC(N)=O)C(=O)c1nccn1Cc1ccccc1] | Compound CCLVIII |
| 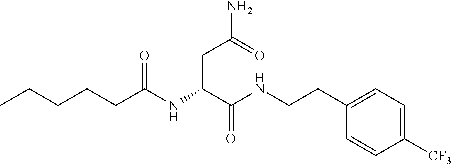 [SMILES: CCCCCC(=O)N[C@H](CC(N)=O)C(=O)NCCc1ccc(C(F)(F)F)cc1] | Compound CCLIX |
| 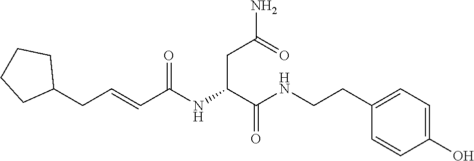 [SMILES: NC(=O)C[C@@H](NC(=O)/C=C/CC1CCCC1)C(=O)NCCc1ccc(O)cc1] | Compound CCLX |
| 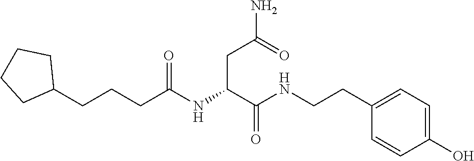 [SMILES: NC(=O)C[C@@H](NC(=O)CCCC1CCCC1)C(=O)NCCc1ccc(O)cc1] | Compound CCLXI |
| 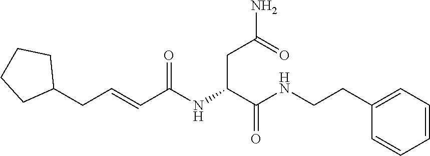 [SMILES: NC(=O)C[C@@H](NC(=O)/C=C/CC1CCCC1)C(=O)NCCc1ccccc1] | Compound CCLXII |
| 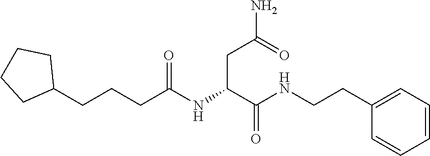 [SMILES: NC(=O)C[C@@H](NC(=O)CCCC1CCCC1)C(=O)NCCc1ccccc1] | Compound CCLXIII |
| 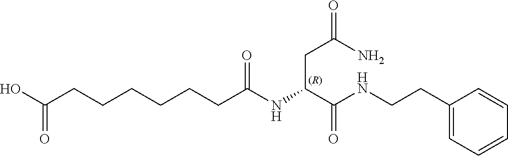 | Compound CCLXIV |
| 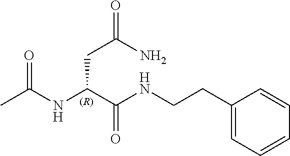 | Compound CCLXV |
| 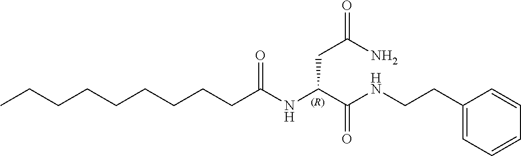 | Compound CCLXVI |
| 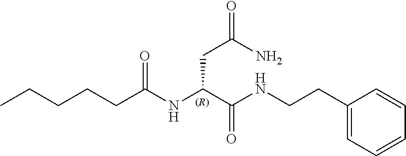 | Compound CCLXVII |
| 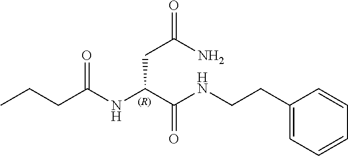 | Compound CCLXVIII |
| 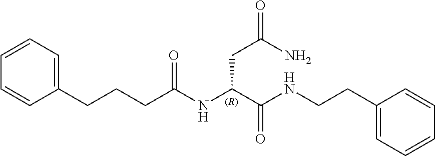 | Compound CCLXIX |
| 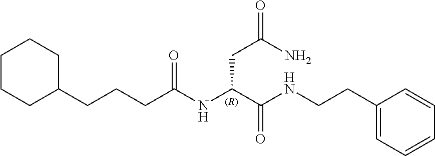 | Compound CCLXX |
| 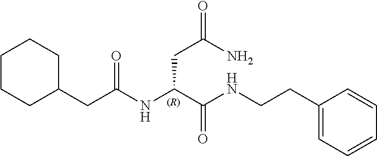 | Compound CCLXXI |
| 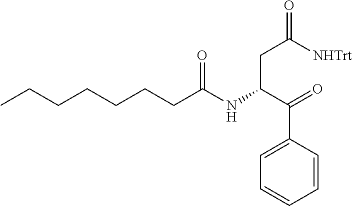 | Compound CCLXXII |
| 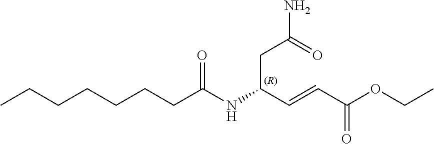 | Compound CCLXXIII |
| 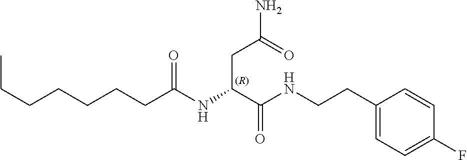 | Compound CCLXXIV |
| 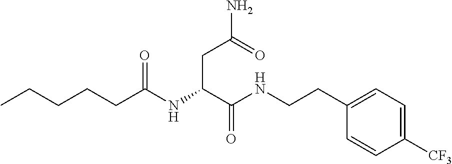 [SMILES: CCCCCC(=O)N[C@H](CC(N)=O)C(=O)NCCc1ccc(C(F)(F)F)cc1] | Compound CCLXXV |
| 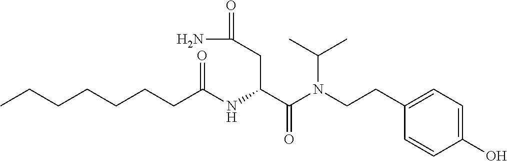 [SMILES: CCCCCCCC(=O)N[C@H](CC(N)=O)C(=O)N(CCc1ccc(O)cc1)C(C)C] | Compound CCLXXVI |
| 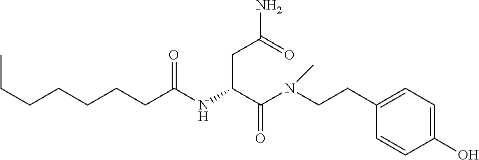 [SMILES: CCCCCCCC(=O)N[C@H](CC(N)=O)C(=O)N(C)CCc1ccc(O)cc1] | Compound CCLXXVII |
| 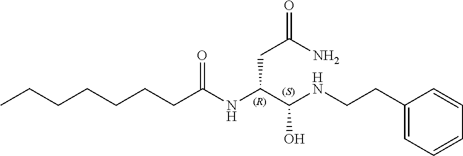 | Compound CCLXXVIII |
| 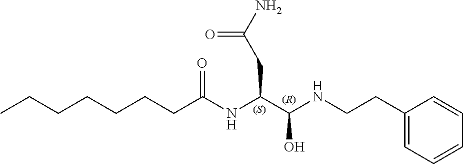 | Compound CCLXXIX |
| 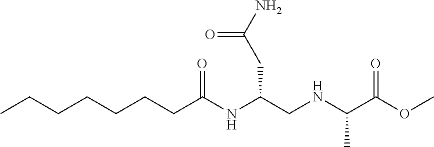 [SMILES: CCCCCCCC(=O)N[C@@H](CN[C@@H](C)C(=O)OC)CC(N)=O] | Compound CCLXXX |
| 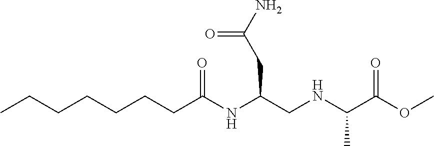 [SMILES: CCCCCCCC(=O)N[C@H](CN[C@@H](C)C(=O)OC)CC(N)=O] | Compound CCLXXXI |
| 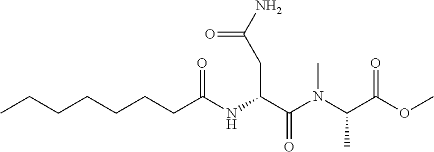 [SMILES: CCCCCCCC(=O)N[C@H](CC(N)=O)C(=O)N(C)[C@@H](C)C(=O)OC] | Compound CCLXXXII |
| 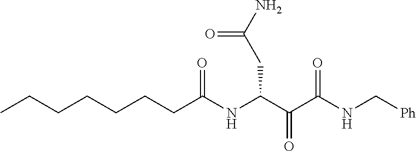 [SMILES: CCCCCCCC(=O)N[C@H](CC(N)=O)C(=O)C(=O)NCc1ccccc1] | Compound CCLXXXIII |

TABLE 5 ¹

| | |
|---|---|
| 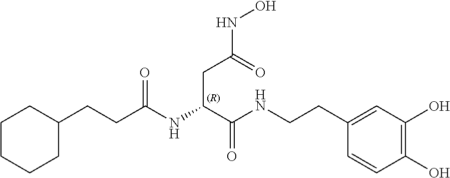 | Compound CCLXXXIV |
| 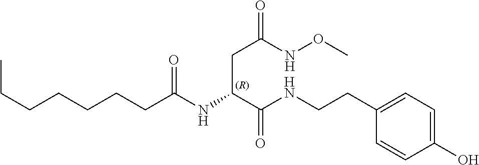 | Compound CCLXXXV |
| 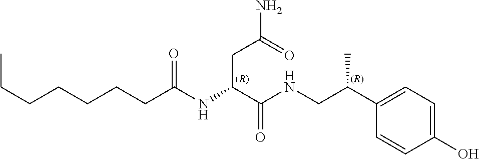 | Compound CCLXXXVI |
| 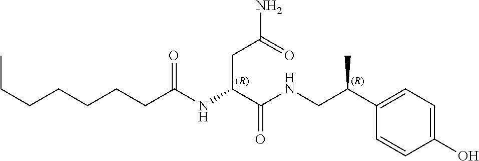 | Compound CCLXXXVII |
| 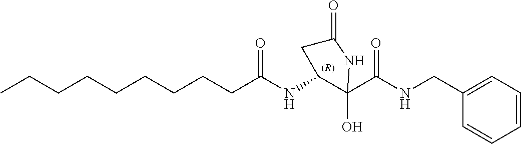 | Compound CCLXXXVIII |
| 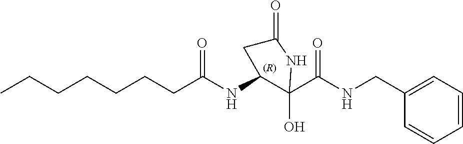 | Compound CCLXXXIX |
| 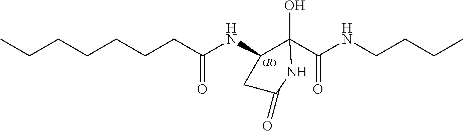 | Compound CCXC |
| 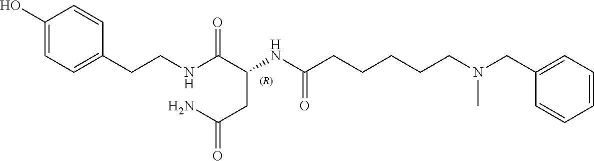 | Compound CCXCI |
| 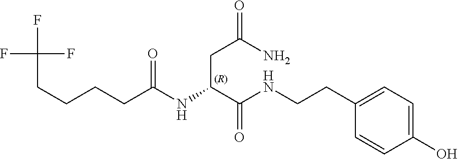 | Compound CCXCII |
| 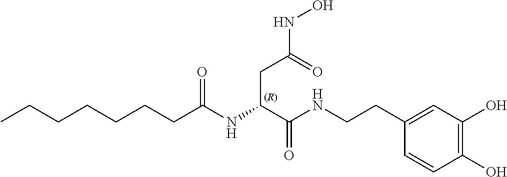 | Compound CCXCIII |
| 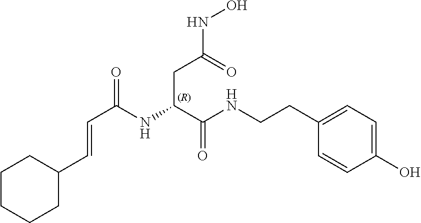 | Compound CCXCIV |
| 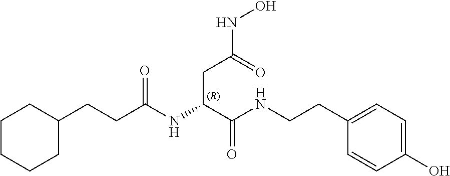 | Compound CCXCV |
| 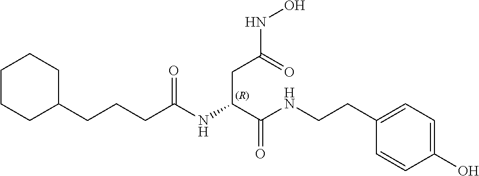 | Compound CCXCVI |
| 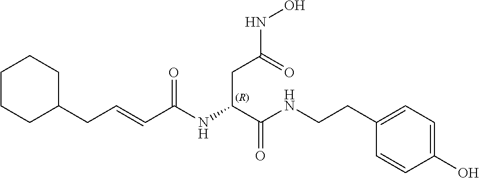 | Compound CCXCVII |
| 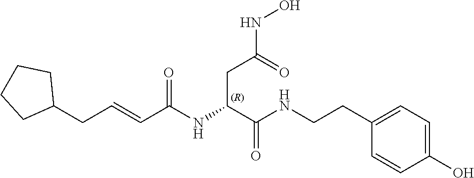 | Compound CCXCVIII |
| 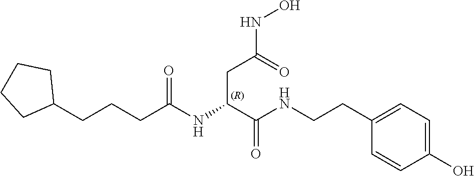 | Compound CCXCIX |
| 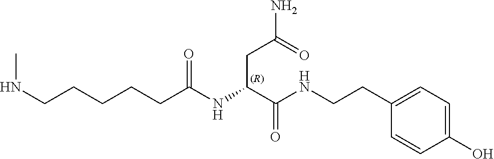 | Compound CCC |
| 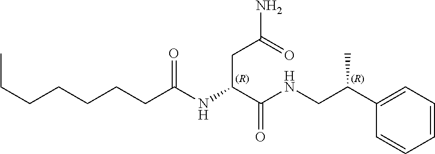 | Compound CCCI |
| 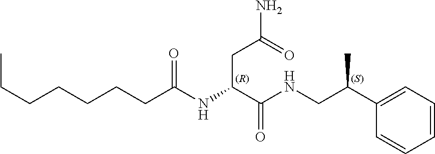 | Compound CCCII |
| 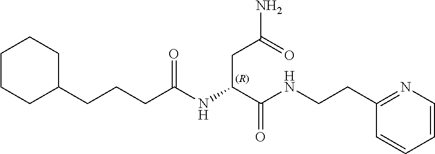 | Compound CCCIII |
| 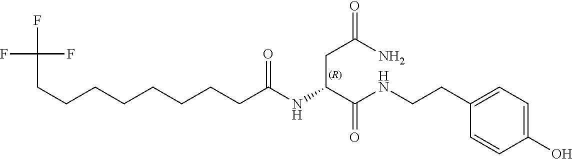 | Compound CCCIV |
| 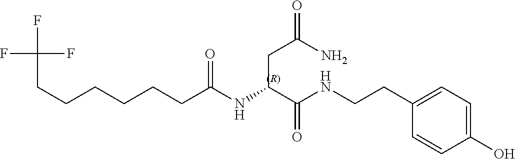 | Compound CCCV |
| 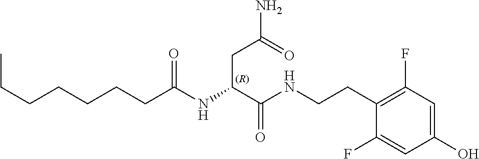 | Compound CCCVI |
| 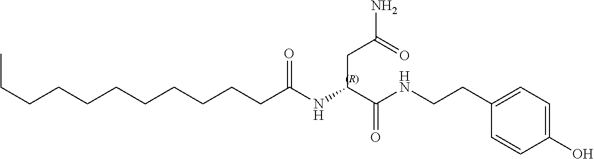 | Compound CCCVII |
| 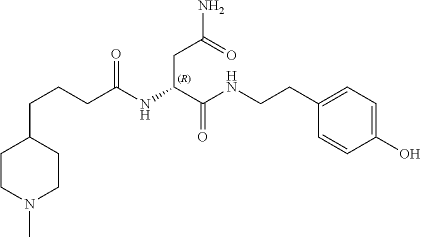 | Compound CCCVIII |
| 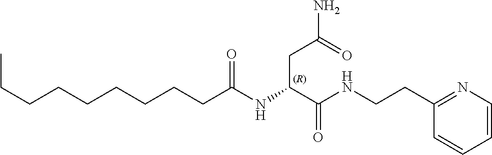 | Compound CCCIX |
| 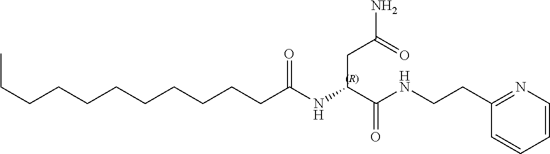 | Compound CCCX |
| 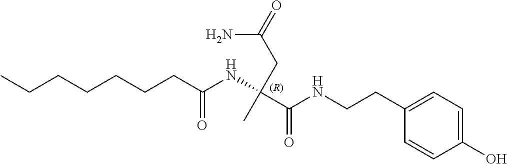 | Compound CCCXI |
| 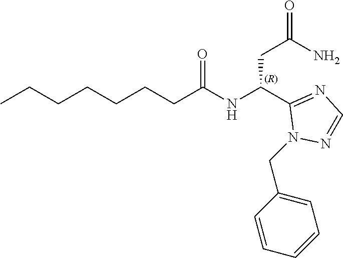 | Compound CCCXII |

¹ Compounds CCLXXXVIII, CCLXXXIX, and CCXC exist in an equilibrium state between the ring open form and the ring closed form depicted in this table.

The compounds of formula (A), pharmaceutically acceptable salts thereof, and solvates of any of the foregoing can be incorporated into pharmaceutical compositions. In some embodiments, the disclosure is drawn to a pharmaceutical composition comprising at least one entity chosen from compounds of formula (A), pharmaceutically acceptable salts thereof, and solvates of any of the foregoing. In some embodiments, the disclosure is drawn to a pharmaceutical composition consisting essentially of at least one entity chosen from compounds of formula (A), pharmaceutically acceptable salts thereof, and solvates of any of the foregoing.

In some embodiments, the pharmaceutical composition comprises at least one entity chosen from compounds of formula (A) pharmaceutically acceptable salts thereof, and solvates of any of the foregoing and at least one pharmaceutically acceptable excipient. Pharmaceutically acceptable excipients are well-known to persons having ordinary skill in the art and are described in, as a non-limiting example, Remington: The Science and Practice of Pharmacy, 22nd Edition, Lippincott Williams & Wilkins, Philadelphia, Pa. (2013) and any other editions, which are hereby incorporated by reference.

Compounds of the disclosure, pharmaceutically acceptable salts thereof, and solvates of any of the foregoing, and/or pharmaceutical compositions comprising said at least one entity chosen from compounds of formula (A) pharmaceutically acceptable salts thereof, and solvates of any of the foregoing can be used in therapeutic treatments.

The compounds, pharmaceutically acceptable salts thereof, solvates of any of the foregoing, and/or pharmaceutical compositions can be administered in unit forms of administration to mammalian subjects, including human beings. Suitable non-limiting examples of unit forms of administration include orally administered forms and forms administered via a parenteral/systemic route, non-limiting examples of which including inhalation, subcutaneous administration, intramuscular administration, intravenous administration, intradermal administration, and intravitreal administration.

In some embodiments, pharmaceutical compositions suitable for oral administration can be in the form of tablets, pills, powders, hard gelatine capsules, soft gelatine capsules, and/or granules. In some embodiments of such pharmaceutical compositions, a compound of the disclosure, pharmaceutically acceptable salt thereof, and/or solvate of any of the foregoing (or are) mixed with one or more inert diluents, non-limiting examples of which including starch, cellulose, sucrose, lactose, and silica. In some embodiments, such pharmaceutical compositions may further comprise one or more substances other than diluents, such as (as non-limiting examples), lubricants, coloring agents, coatings, or varnishes.

In some embodiments, pharmaceutical compositions for parenteral administration can be in the form of aqueous solutions, non-aqueous solutions, suspensions, emulsions, drops, or any combination(s) thereof. In some embodiments, such pharmaceutical compositions may comprise one or more of water, pharmaceutically acceptable glycol(s), pharmaceutically acceptable oil(s), pharmaceutically acceptable organic esters, or other pharmaceutically acceptable solvents.

In some embodiments, disclosed herein is a method of inhibiting ClbP comprising administering to a subject in need thereof at least one entity chosen from compounds of formula (A), pharmaceutically acceptable salts thereof, and solvates of any of the foregoing. In some embodiments, disclosed herein is a method of reducing the activity of ClbP comprising administering to a subject in need thereof at least one entity chosen from compounds of formula (A), pharmaceutically acceptable salts thereof, and solvates of any of the foregoing.

In some embodiments, disclosed herein is a method of treating a cancer comprising administering to a subject in need thereof at least one entity chosen from compounds of formula (A), pharmaceutically acceptable salts thereof, and solvates of any of the foregoing. In some embodiments, the cancers are chosen from liquid tumors and solid tumors. In some embodiments, the cancer is colorectal cancer. In some embodiments, the cancer is chosen from breast cancers, respiratory tract cancers, brain cancers, cancers of reproductive organs, digestive tract cancers, urinary tract cancers, eye cancers, liver cancers, skin cancers, head and neck cancers, thyroid cancers, parathyroid cancers, and metastases of any of the foregoing. In some embodiments, the cancers are chosen from breast cancers, pancreatic cancers, prostate cancers, and colon cancers. In some embodiments, the cancers are chosen from lymphomas, sarcomas, and leukemias.

In some embodiments, disclosed herein is a method of treating ocular disorders comprising administering to a subject in need thereof at least one entity chosen from compounds of formula (A), pharmaceutically acceptable salts thereof, and solvates of any of the foregoing.

With regard to the methods disclosed herein, the mode (or modes) of administration, dose (or doses), and pharmaceutical form (or forms) can be determined according to criteria generally considered during the establishment of a treatment of a patient, such as, by way of non-limiting examples, the potency of the compound(s) and/or pharmaceutically acceptable salts of the compound(s), the age of the patient, the body weight of the patient, the severity of the patient's condition (or conditions), the patient's tolerance to the treatment, and secondary effects observed in treatment. Determination of doses effective to provide therapeutic benefit for specific modes and frequency of administration is within the capabilities of those skilled in the art.

In some embodiments, a compound of formula (A), pharmaceutically acceptable salts thereof, and solvates of any of the foregoing is present in a pharmaceutical composition in an amount ranging from 5 μg to 2,000 mg. In some embodiments, a compound of the disclosure, pharmaceutically acceptable salt thereof, and/or solvate of any of the foregoing is present in a pharmaceutical composition in an amount ranging from 5 μg to 1,000 mg. In some embodiments, a compound of the disclosure, pharmaceutically acceptable salt thereof, and/or solvate of any of the foregoing is present in a pharmaceutical composition in an amount ranging from 5 μg to 500 mg. In some embodiments, a compound of the disclosure, pharmaceutically acceptable salt thereof, and/or solvate of any of the foregoing is present in a pharmaceutical composition in an amount ranging from 5 μg to 250 mg. In some embodiments, a compound of the disclosure, pharmaceutically acceptable salt thereof, and/or solvate of any of the foregoing is present in a pharmaceutical composition in an amount ranging from 5 μg to 100 mg. In some embodiments, a compound of the disclosure, pharmaceutically acceptable salt thereof, and/or solvate of any of the foregoing is present in a pharmaceutical composition in an amount ranging from 5 μg to 50 mg.

In some embodiments, a compound of the disclosure, pharmaceutically acceptable salt thereof, and/or solvate of any of the foregoing is present in a pharmaceutical composition in an amount ranging from 1 mg to 5,000 mg. In some embodiments, a compound of the disclosure, pharmaceutically acceptable salt thereof, and/or solvate of any of the foregoing thereof is present in a pharmaceutical composition in an amount ranging from 1 mg to 3,000 mg. In some embodiments, a compound of the disclosure, pharmaceutically acceptable salt thereof, and/or solvate of any of the foregoing is present in a pharmaceutical composition in an amount ranging from 1 mg to 2,000 mg. In some embodiments, a compound of the disclosure, pharmaceutically acceptable salt thereof, and/or solvate of any of the foregoing is present in a pharmaceutical composition in an amount ranging from 1 mg to 1,000 mg. In some embodiments, a compound of the disclosure, pharmaceutically acceptable salt thereof, and/or solvate of any of the foregoing is present in a pharmaceutical composition in an amount ranging from 1 mg to 500 mg. In some embodiments, a compound of the disclosure, pharmaceutically acceptable salt thereof, and/or solvate of any of the foregoing is present in a pharmaceutical composition in an amount ranging from 1 mg to 250 mg. In some embodiments, a compound of the disclosure, pharmaceutically acceptable salt thereof, and/or solvate of any of the foregoing is present in a pharmaceutical composition in an amount ranging from 1 mg to 100 mg. In some embodiments, a compound of the disclosure, pharmaceutically acceptable salt thereof, and/or solvate of any of the foregoing is present in a pharmaceutical composition in an amount ranging from 1 mg to 50 mg.

In some embodiments, a compound of the disclosure, pharmaceutically acceptable salt thereof, and/or solvate of any of the foregoing is present in a pharmaceutical composition in an amount of 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 125 mg, 150 mg, 175 mg, 200 mg, 225 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1,000 mg, 1,100 mg, 1,200 mg, 1,300 mg, 1,400 mg, 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, 3,000 mg, 3,100 mg, 3,200 mg, 3,300 mg, 3,400 mg, 3,500 mg, 3,600 mg, 3,700 mg, 3,800 mg, 3,900 mg, 4,000 mg, 4,100 mg, 4,200 mg, 4,300 mg, 4,400 mg, 4,500 mg, 4,600 mg, 4,700 mg, 4,800 mg, 4,900 mg, or 5,000 mg.

Effective amounts and dosages can be estimated initially from in vitro assays. For example, an initial dosage for use in animals can be formulated to achieve a circulating blood or serum concentration of active compound that is at or above an IC₅₀ of the particular compound as measured in an in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” in Goodman and Gilman's The Pharmaceutical Basis of Therapeutics, Chapter 1, pp. 1-46, latest edition, Pergamagon Press, and the references cited therein, which methods are incorporated herein by reference in their entirety. Initial dosages can also be estimated from in vivo data, such as animal models. Animal models useful for testing the efficacy of compounds to treat or prevent the various diseases described in this disclosure are well-known in the art.

In some embodiments, the administered dose ranges from 0.0001 or 0.001 or 0.01 mg/kg/day to 100 mg/kg/day, but can be higher or lower, depending upon, among other factors, the activity of the compound, its bioavailability, the mode of administration and various factors discussed above. Doses and intervals can be adjusted individually to provide plasma levels of the compound(s) which are sufficient to maintain therapeutic or prophylactic effect. For example, the compounds can be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Non-limiting embodiments of the present disclosure include:

Embodiment 1. A compound of Formula (A):

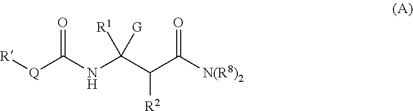

a pharmaceutically acceptable salt thereof, or a solvate of any of the foregoing,
R′ is chosen from hydrogen, C₁₋₁₄ alkyl and C₁₋₁₄ alkenyl, wherein alkyl and alkenyl are optionally substituted;
Q is CR″R″′, NR″, or O;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl; and
G is chosen from C(═O)CF₃, C(═O)H, B(OH)₂, B(Oalkyl)₂, CN, P(═O)(Oaryl)₂, P(═O)(OCH₃)F, S(═O)₂F, C(═O)aryl, C(═O)heteroaryl,

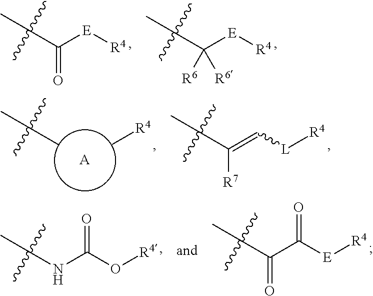

wherein alkyl, aryl, and heteroaryl are optionally substituted;
wherein:
E is chosen from CR³R³′, NR³, and O, wherein optionally R¹ and R³ or R² and R³ together form a ring;
L is chosen from S(═O)₂, C(═O), C(═O)NR³, and a bond;
R³ and R^{3′} each independently is chosen from H, halogen, and C₁₋₃ alkyl;
Ring A is chosen from 4-8 membered heteroaryl and 4-8 membered heterocyclyl; wherein the 4-8 membered heteroaryl or 4-8 membered heterocyclyl is optionally substituted;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted;
R^{4′} is chosen from C₁₋₇ alkyl and C₀₋₆ alkyl-R⁵; wherein the C₁₋₇ alkyl or C₀₋₆ alkyl-R⁵ is optionally substituted;
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted;
R⁶ and R^{6′} each independently is chosen from H, OH, haloalkyl, and C₁₋₃ alkyl, or R⁶ and R^{6′} together form a 4-6 membered ring; and
R⁷ is chosen from H, C₁₋₃ alkyl, and halogen.
   Embodiment 2. The compound of embodiment 1, wherein R′ is heptyl.
   Embodiment 3. The compound of embodiment 1 or 2, wherein Q is NH.
   Embodiment 4. The compound of embodiment 1 or 2, wherein Q is CH₂.
   Embodiment 5. A compound of embodiment 1 chosen from:

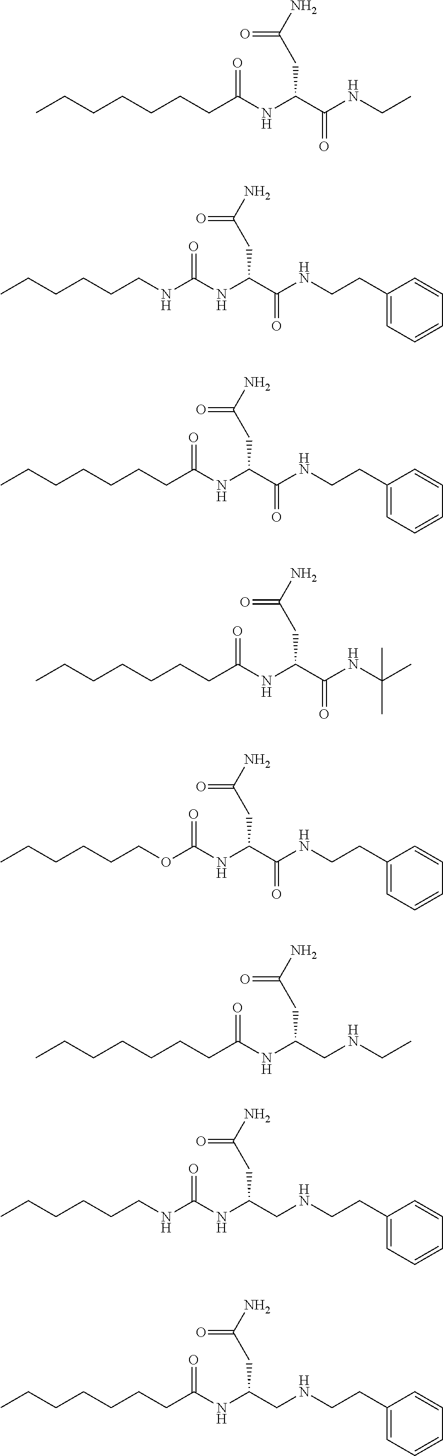

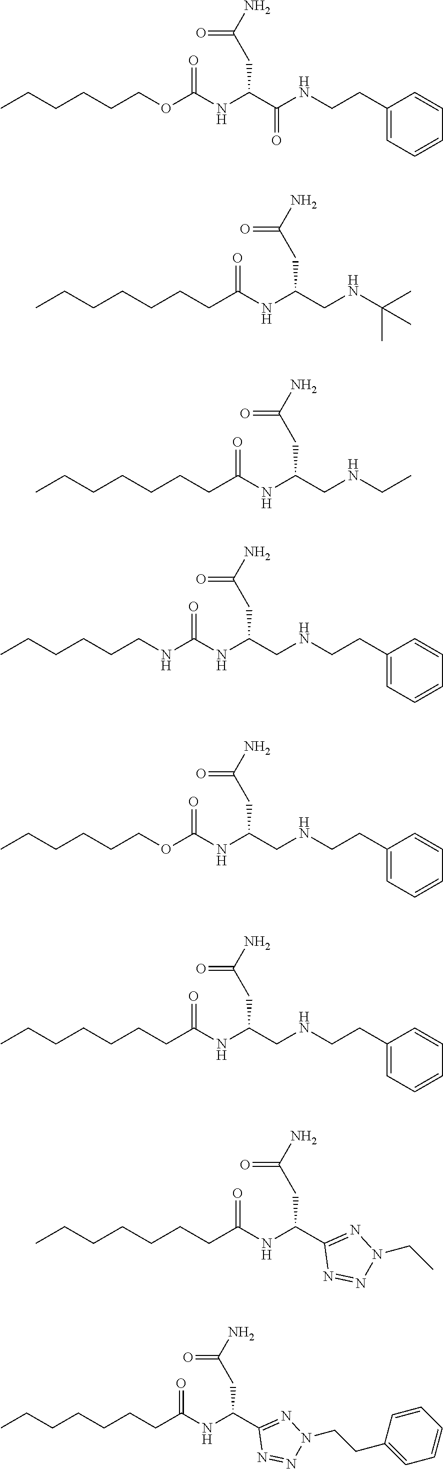

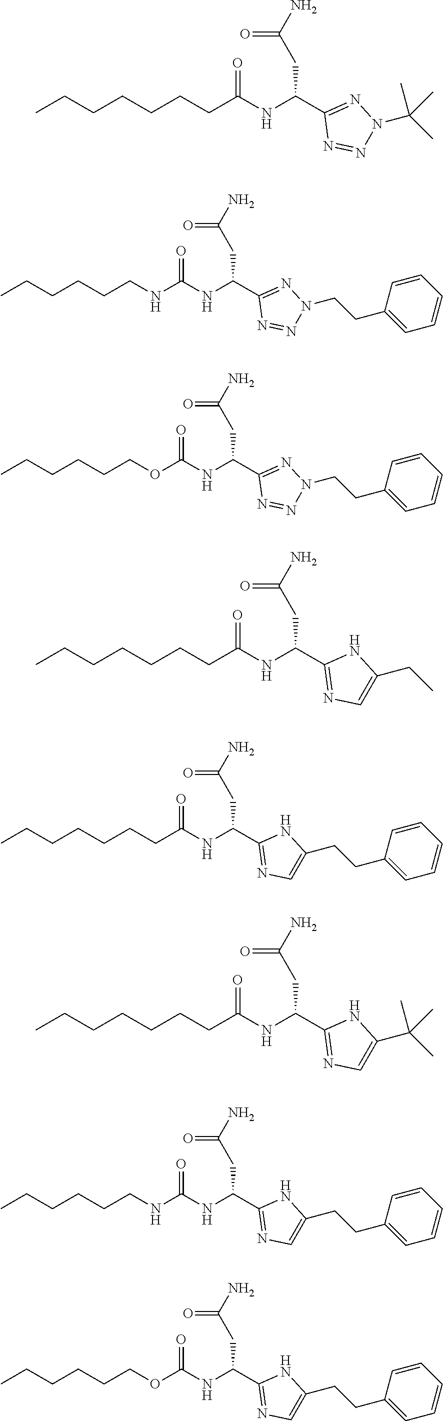

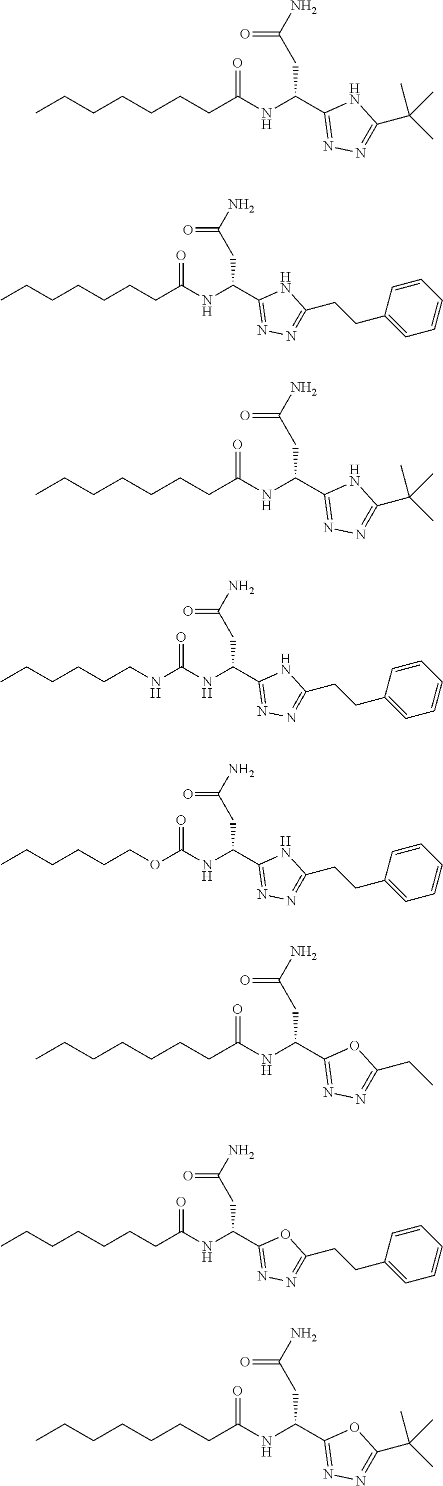

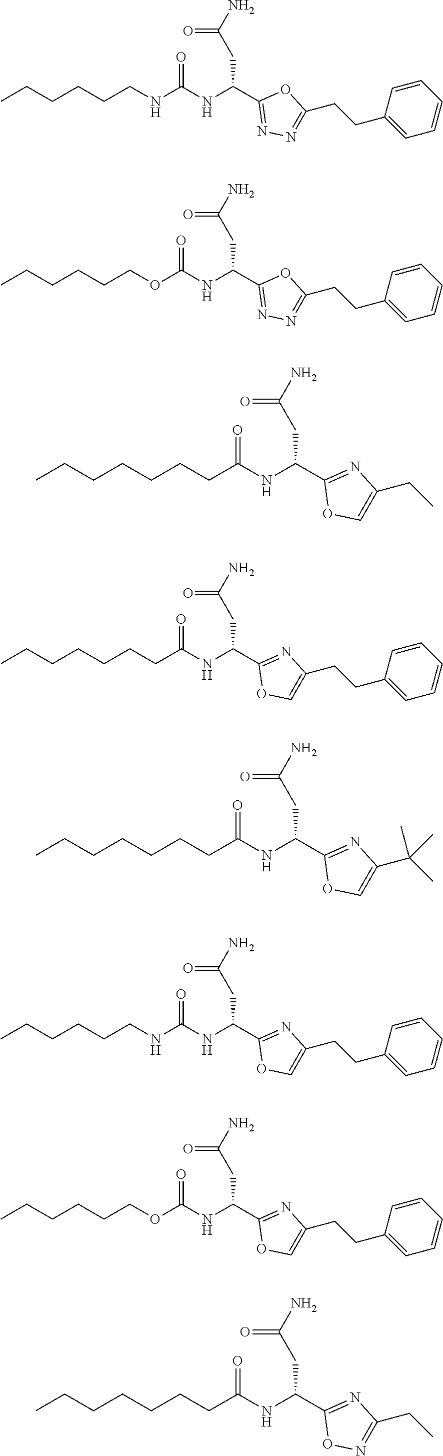

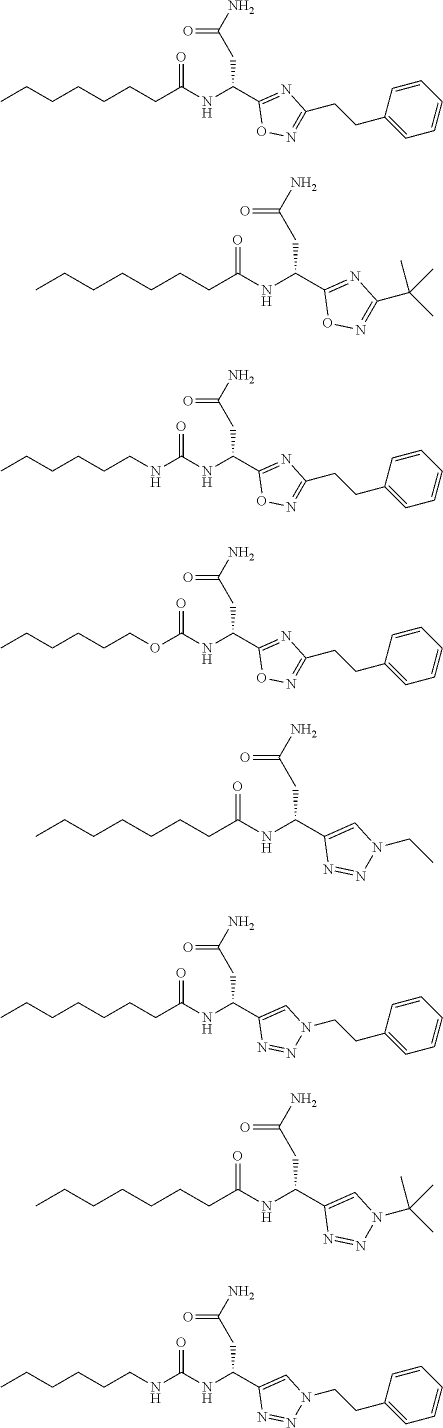

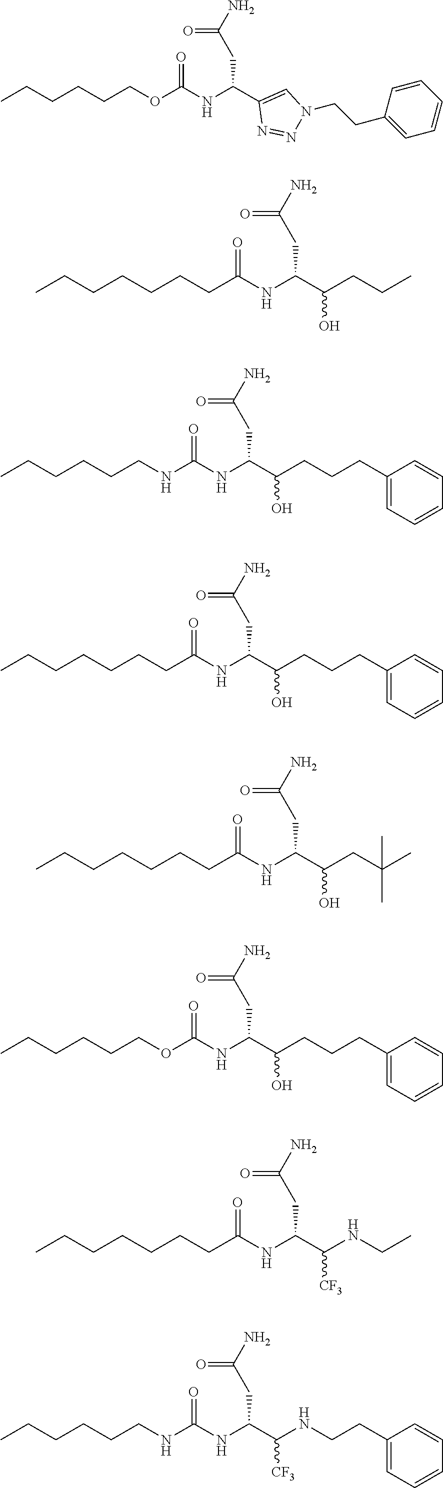

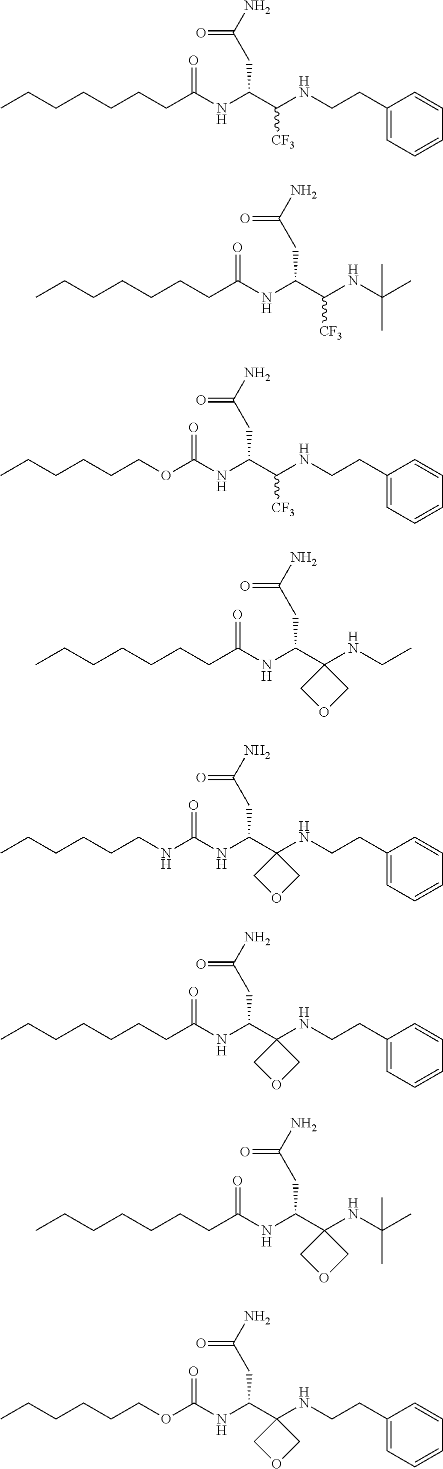

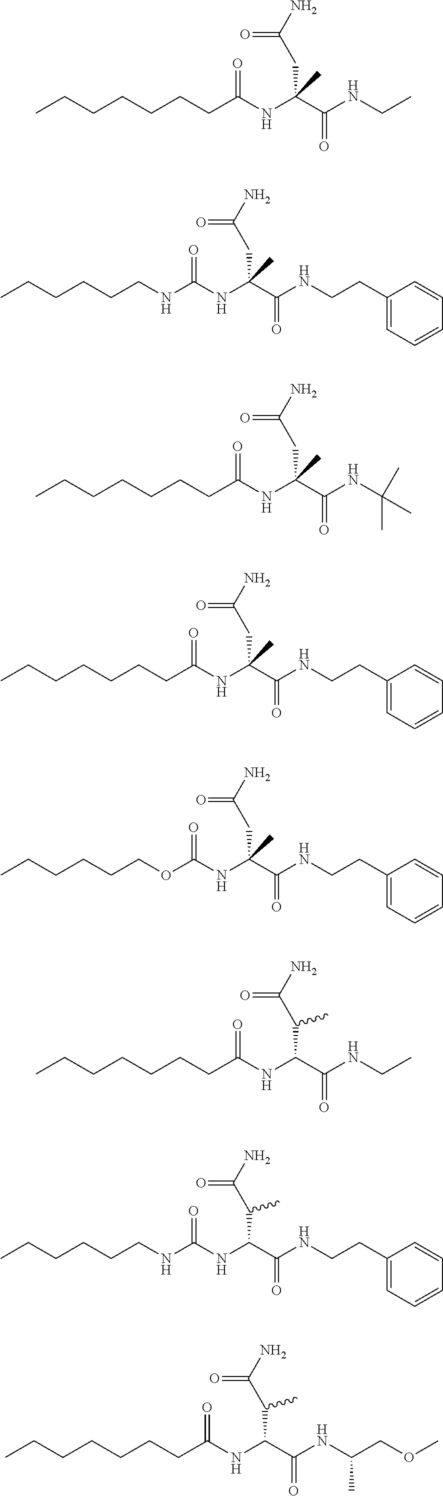

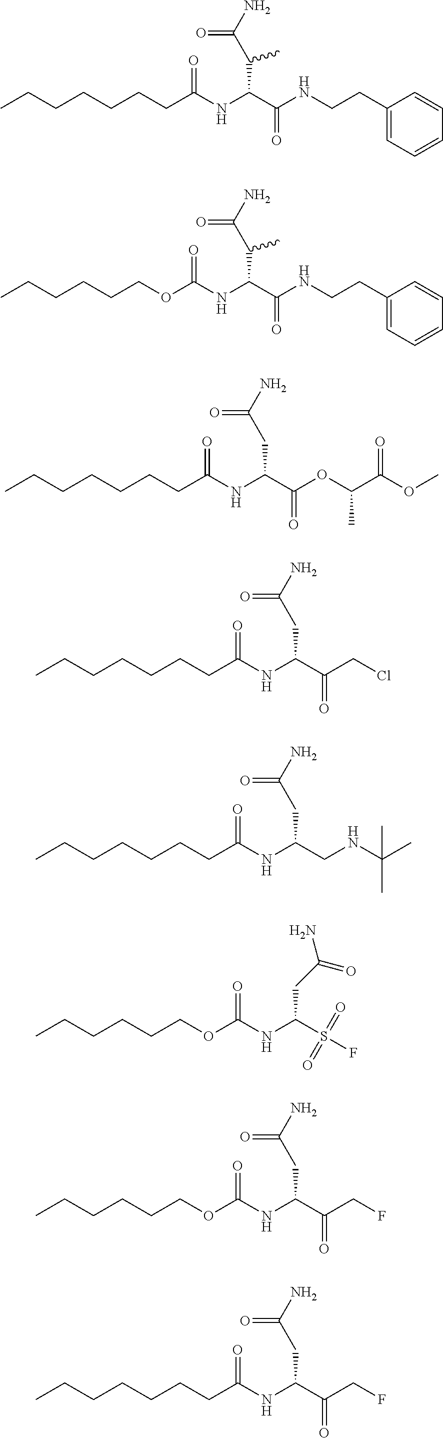

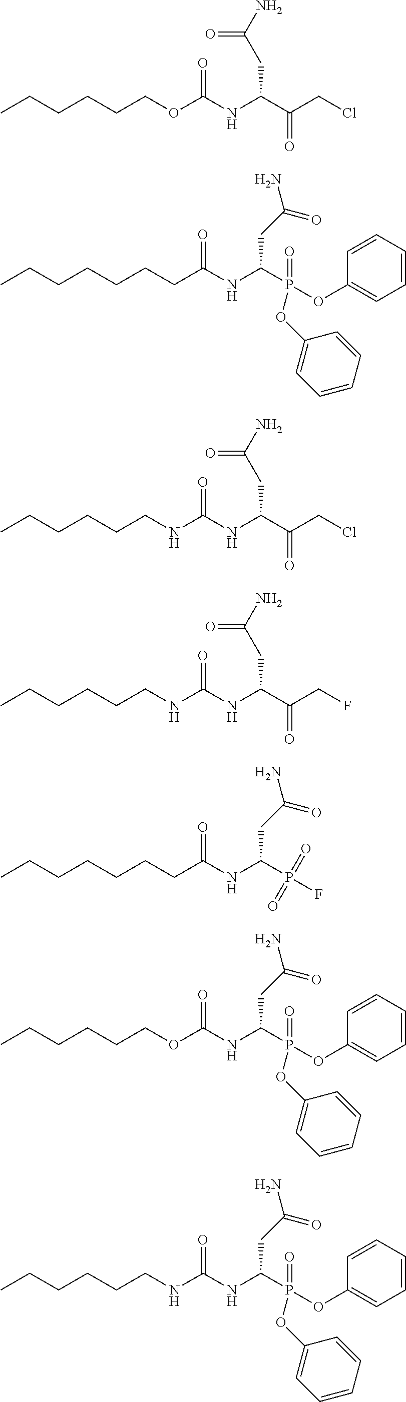

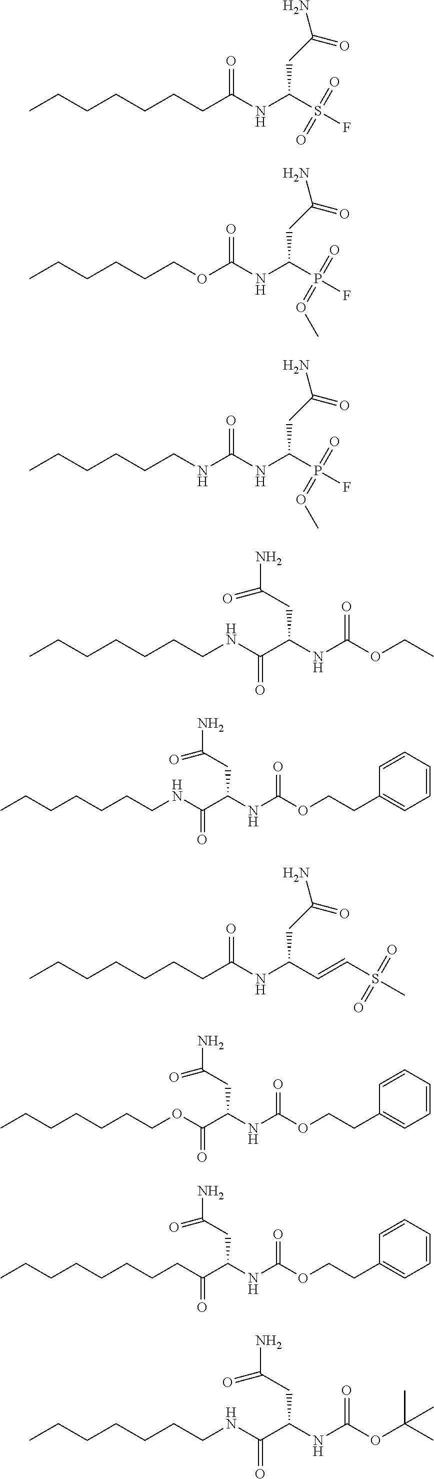

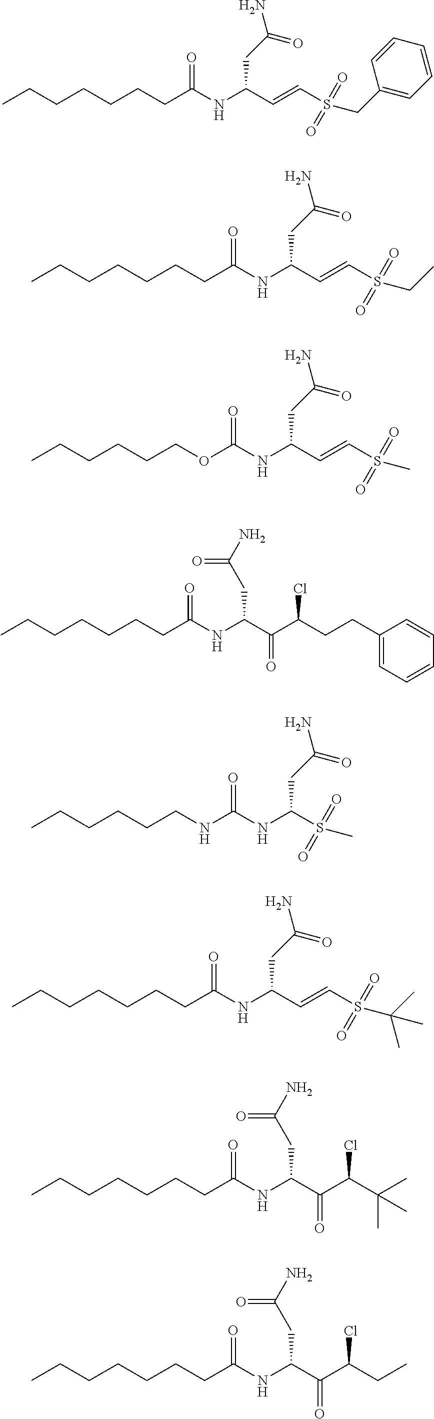

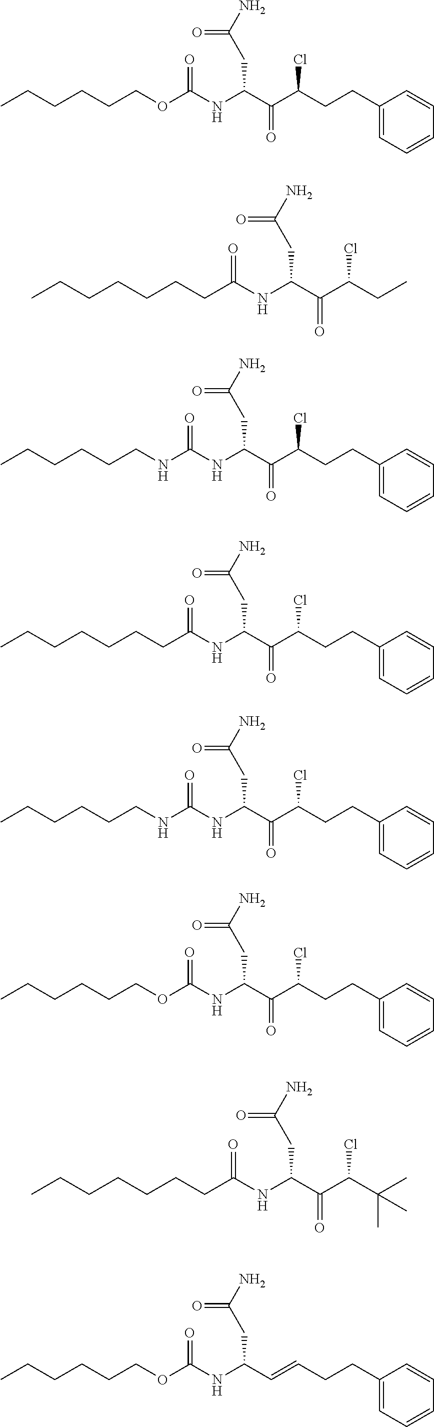

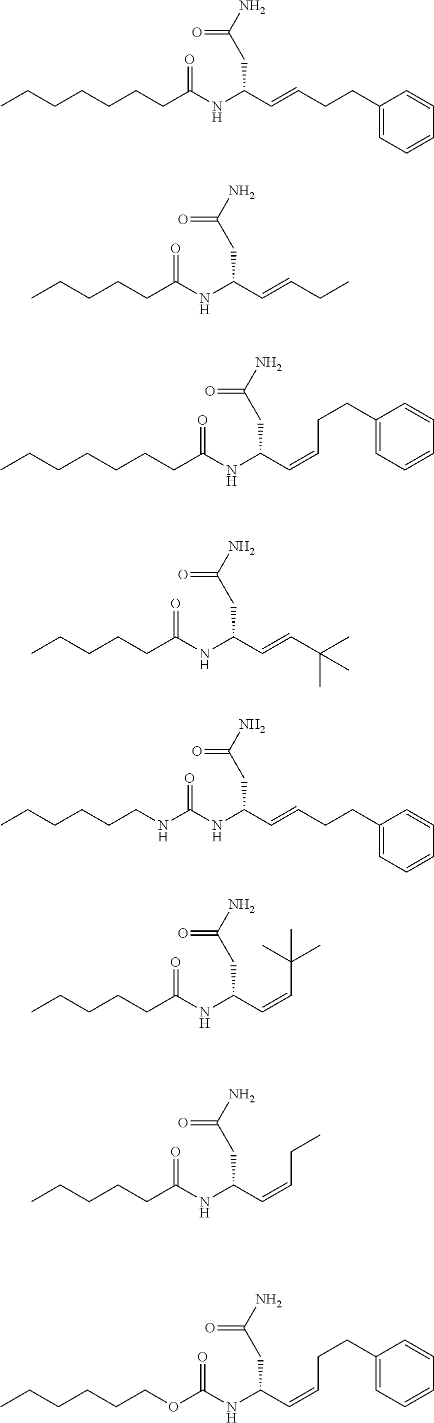

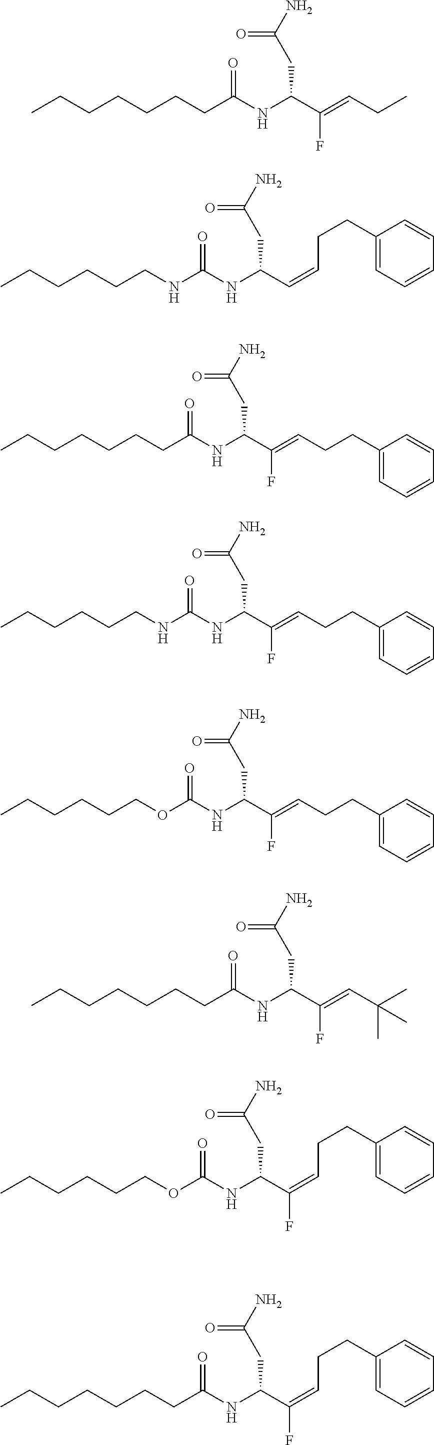

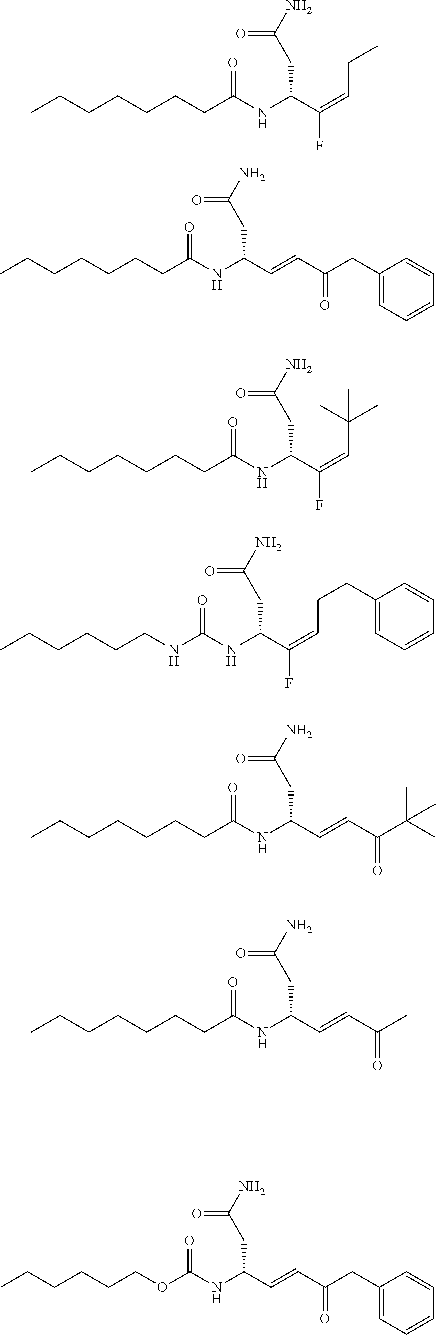

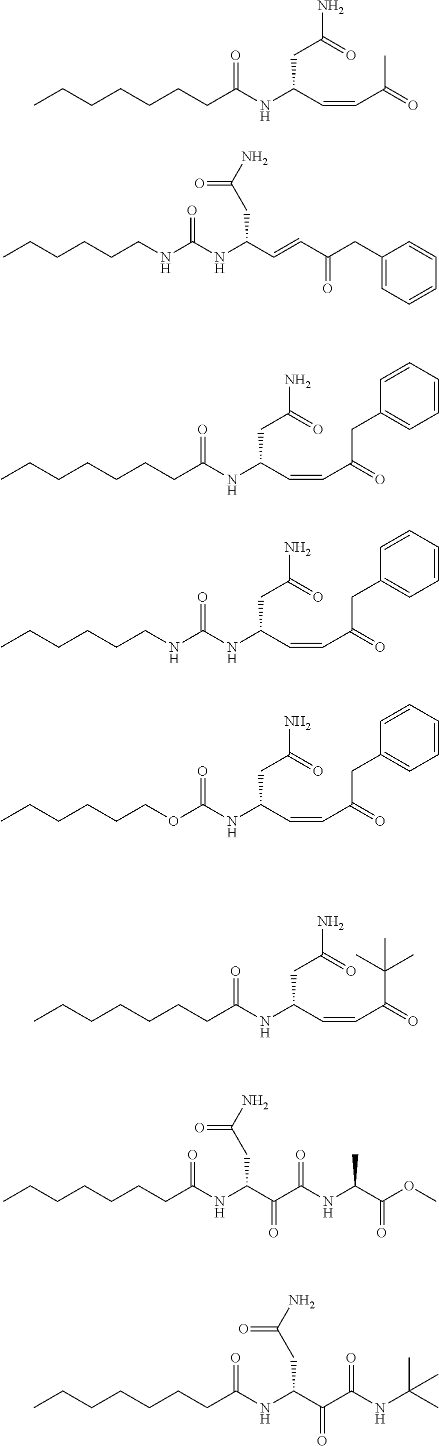

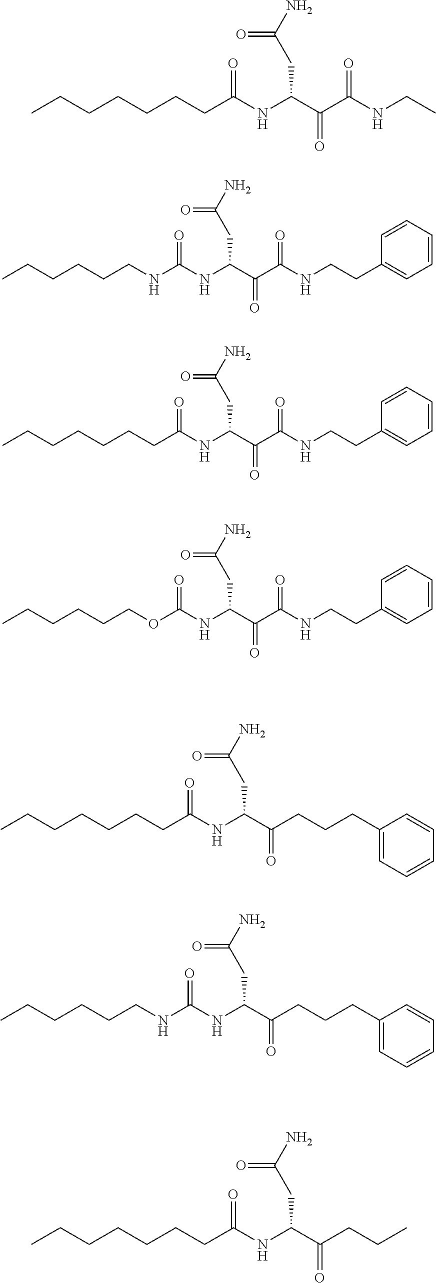

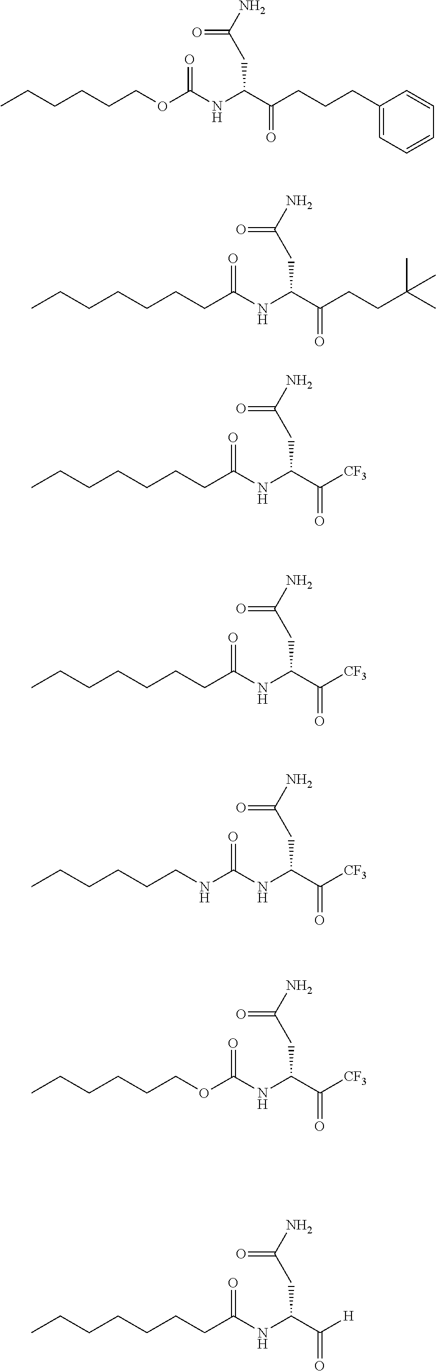

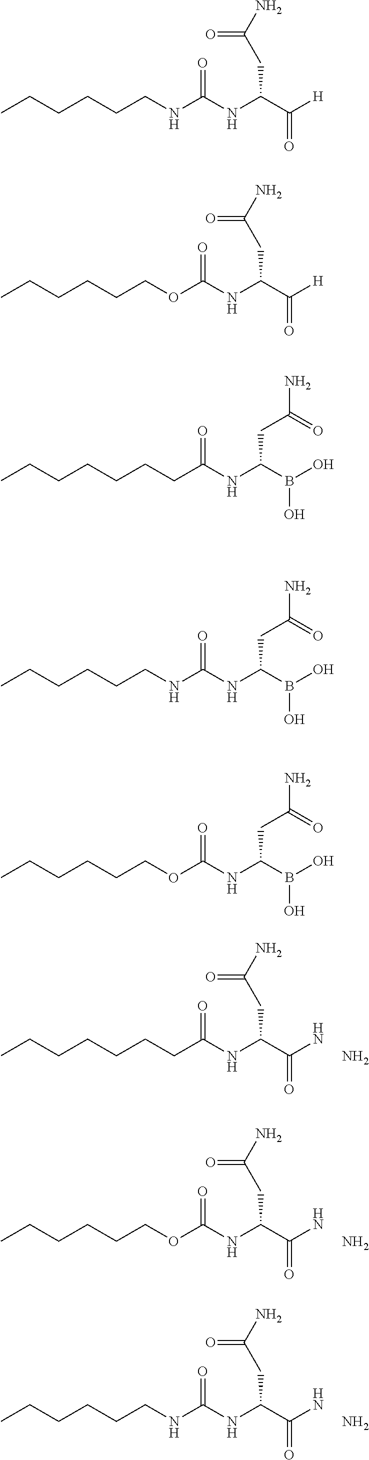

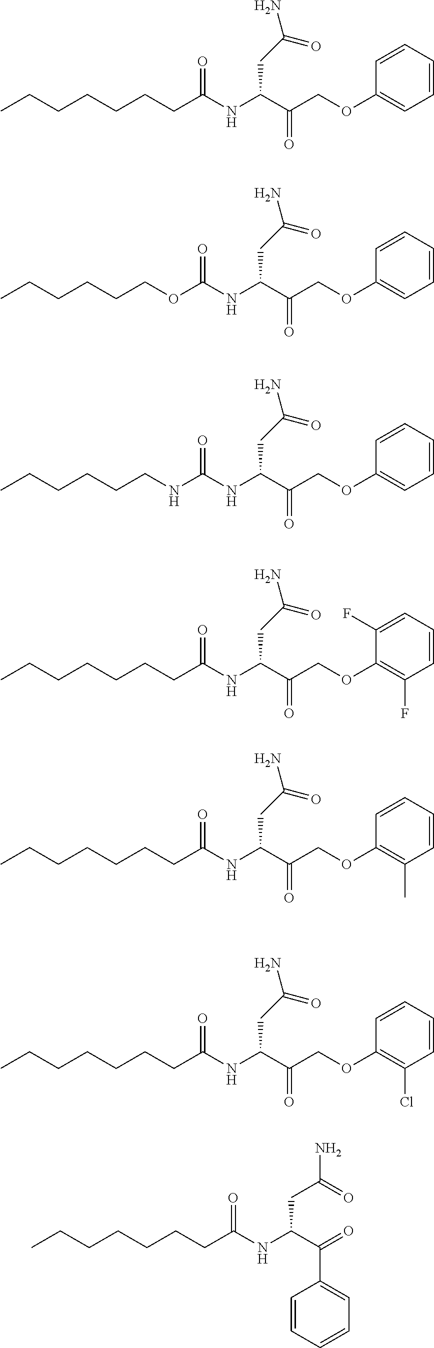

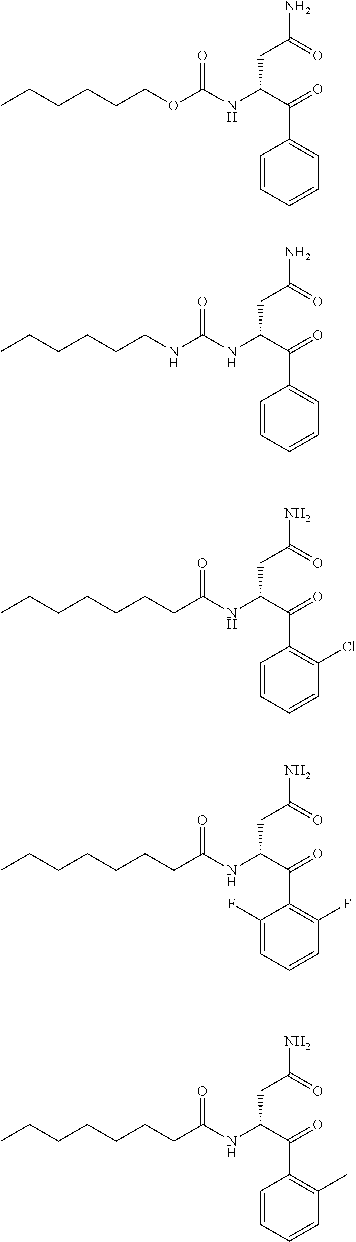

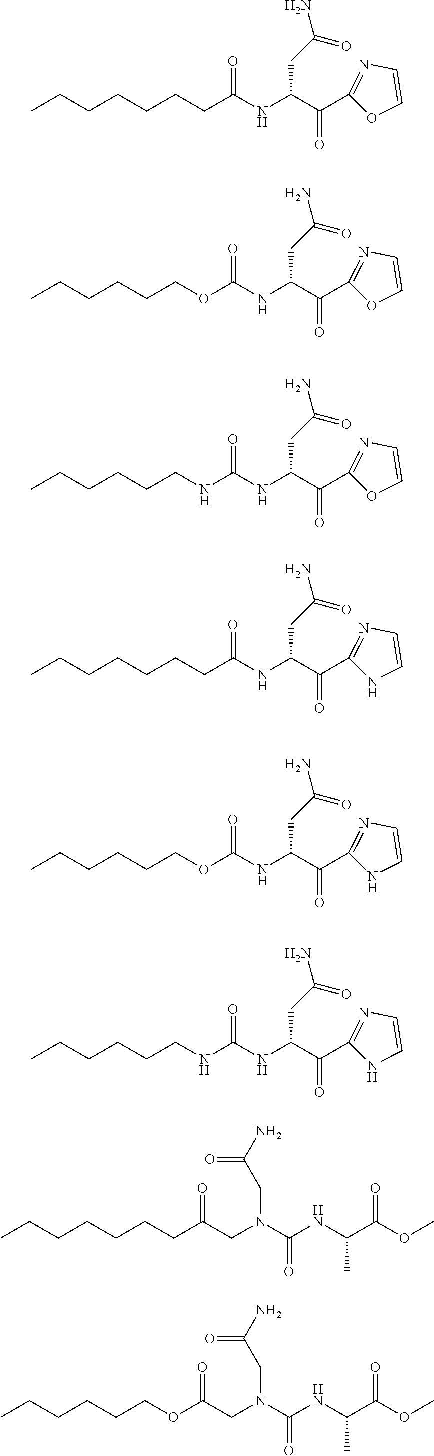

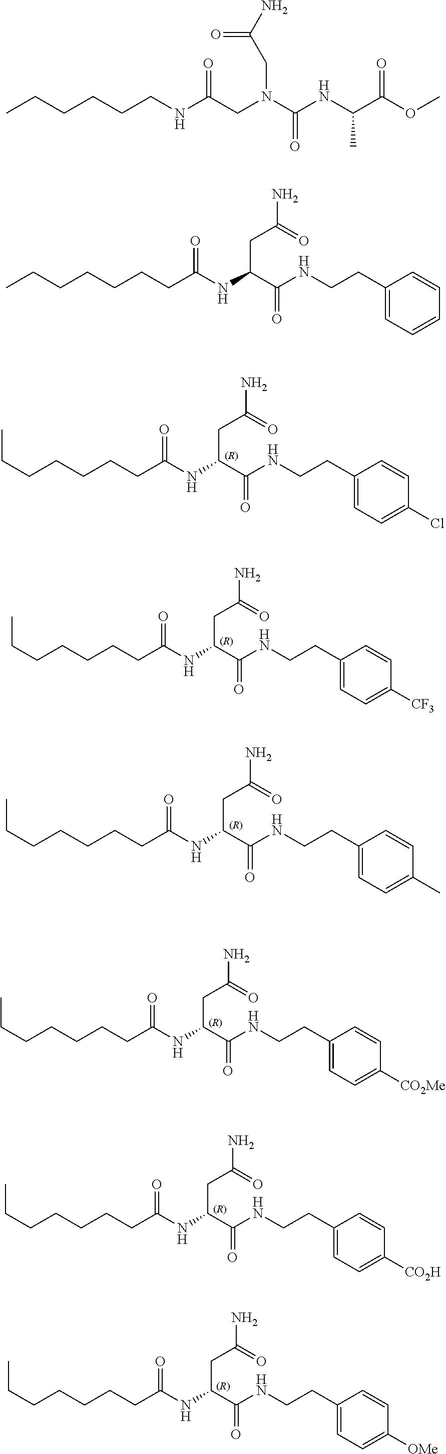

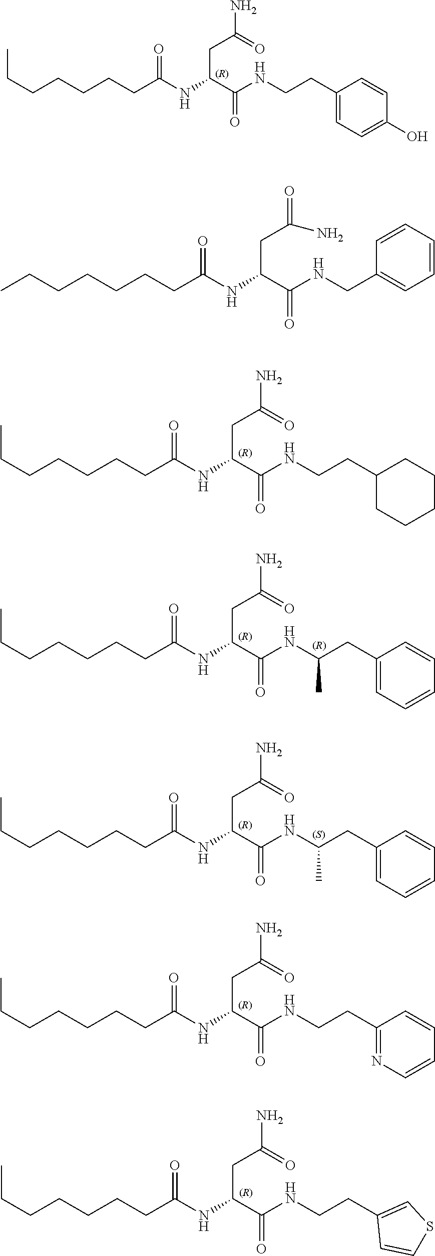

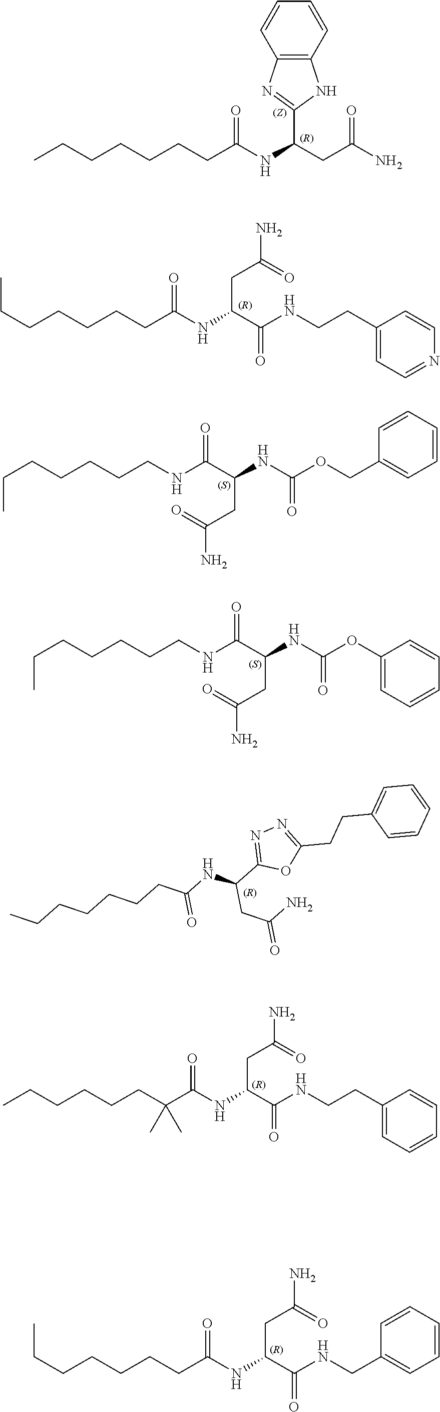

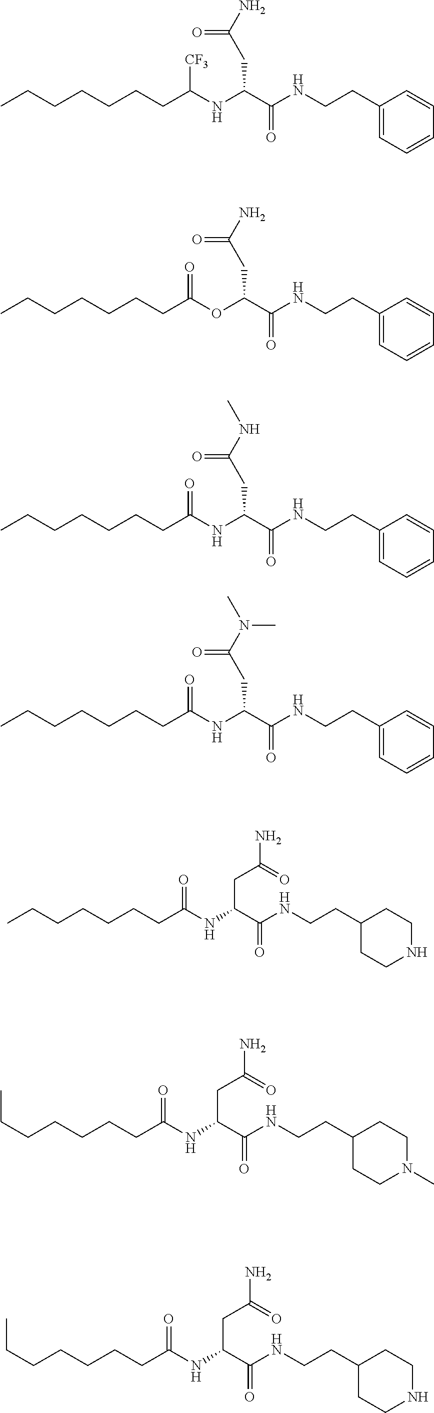

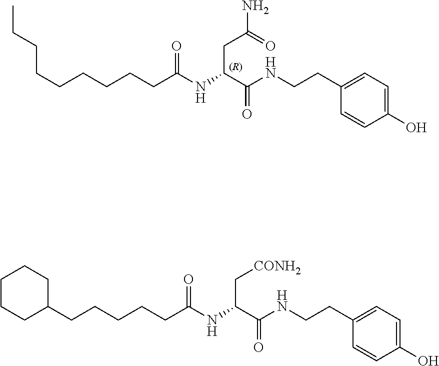

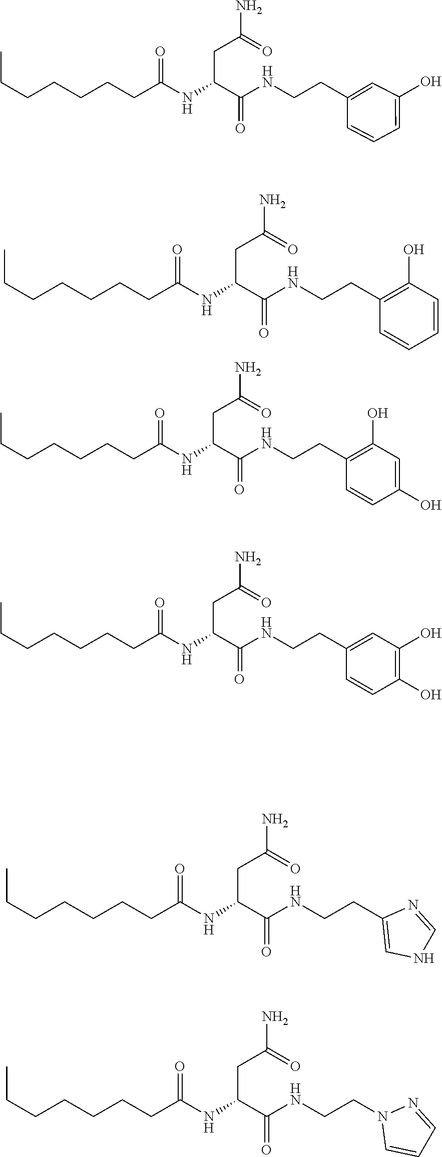

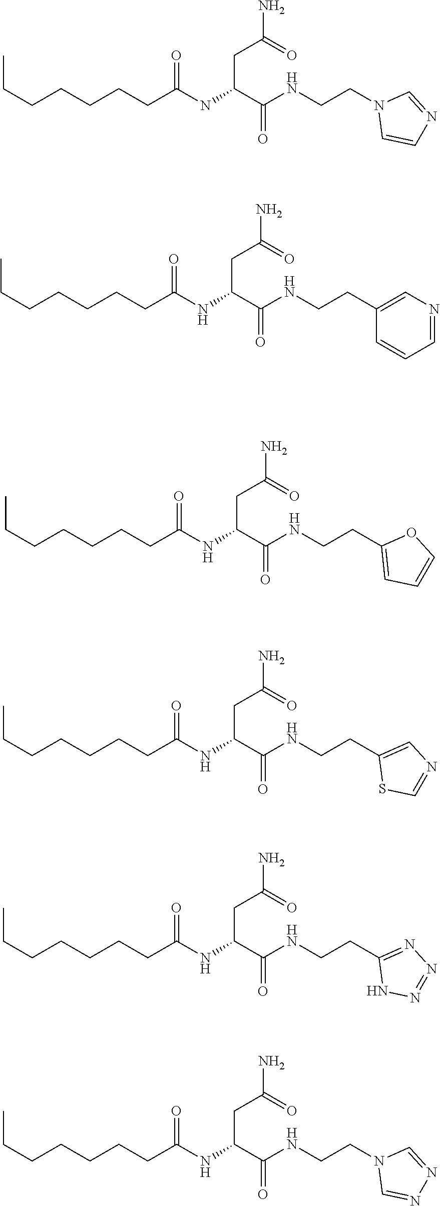

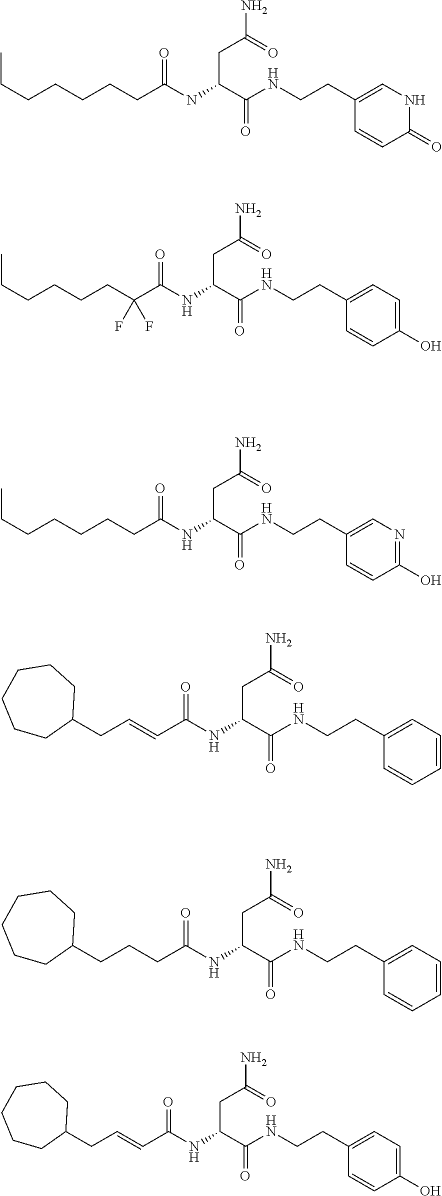 [SMILES: CCCCCCCC(=O)N[C@H](CC(N)=O)C(=O)NCCc1ccc(=O)[nH]c1 ; CCCCCCC(F)(F)C(=O)N[C@H](CC(N)=O)C(=O)NCCc1ccc(O)cc1 ; CCCCCCCC(=O)N[C@H](CC(N)=O)C(=O)NCCc1ccc(O)nc1 ; NC(=O)C[C@@H](NC(=O)/C=C/CC1CCCCCC1)C(=O)NCCc1ccccc1 ; NC(=O)C[C@@H](NC(=O)CCCC1CCCCCC1)C(=O)NCCc1ccccc1 ; NC(=O)C[C@@H](NC(=O)/C=C/CC1CCCCCC1)C(=O)NCCc1ccc(O)cc1]

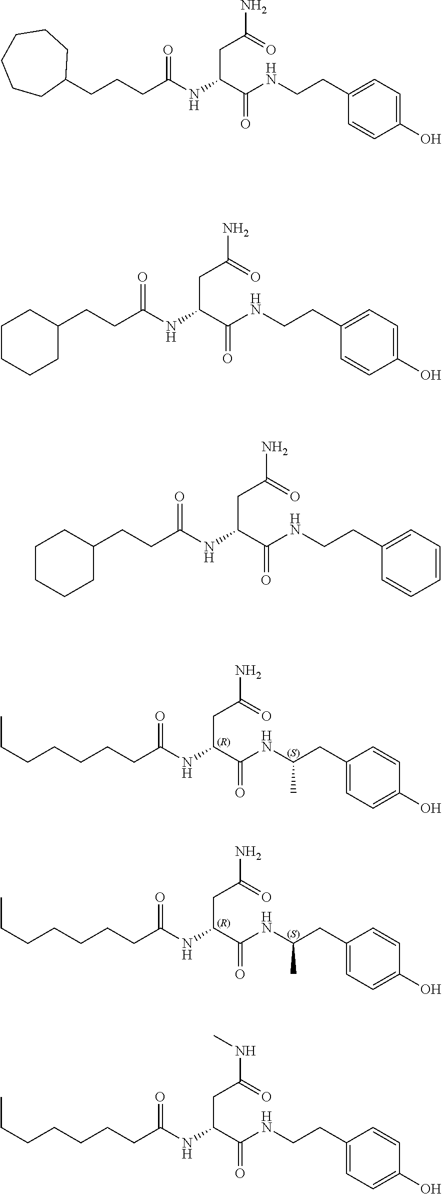

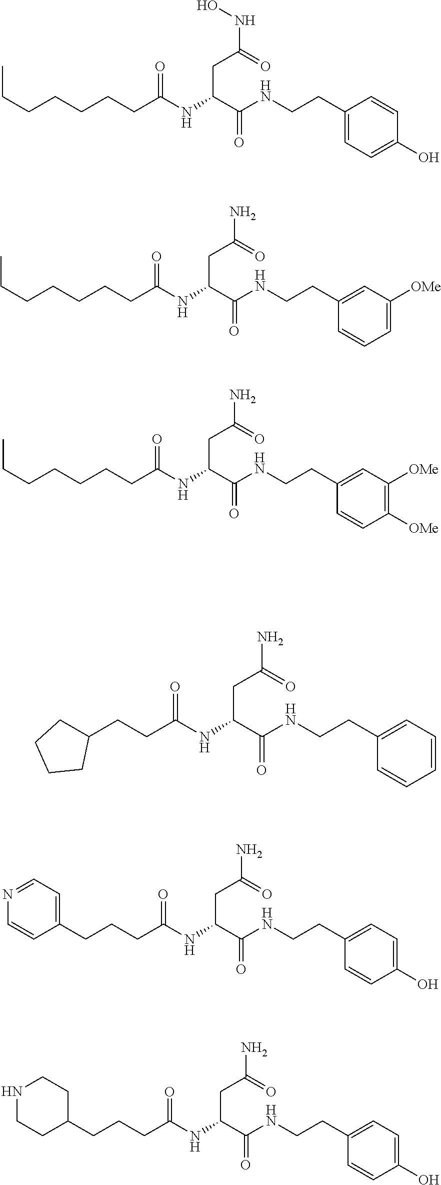 [SMILES: CCCCCCCC(=O)N[C@H](CC(=O)NO)C(=O)NCCc1ccc(O)cc1 ; CCCCCCCC(=O)N[C@H](CC(N)=O)C(=O)NCCc1cccc(OC)c1 ; CCCCCCCC(=O)N[C@H](CC(N)=O)C(=O)NCCc1ccc(OC)c(OC)c1 ; NC(=O)C[C@@H](NC(=O)CCC1CCCC1)C(=O)NCCc1ccccc1 ; NC(=O)C[C@@H](NC(=O)CCCc1ccncc1)C(=O)NCCc1ccc(O)cc1 ; NC(=O)C[C@@H](NC(=O)CCCC1CCNCC1)C(=O)NCCc1ccc(O)cc1]

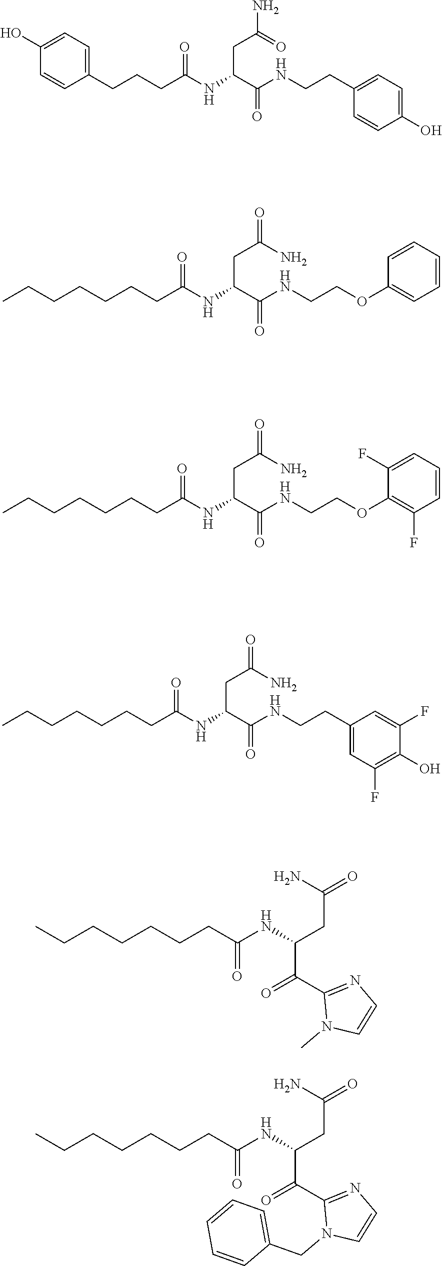

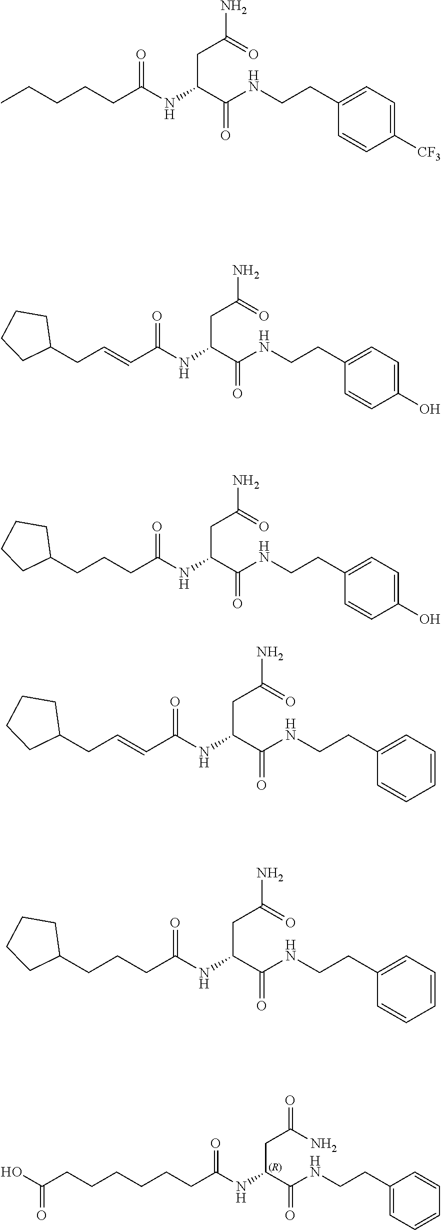

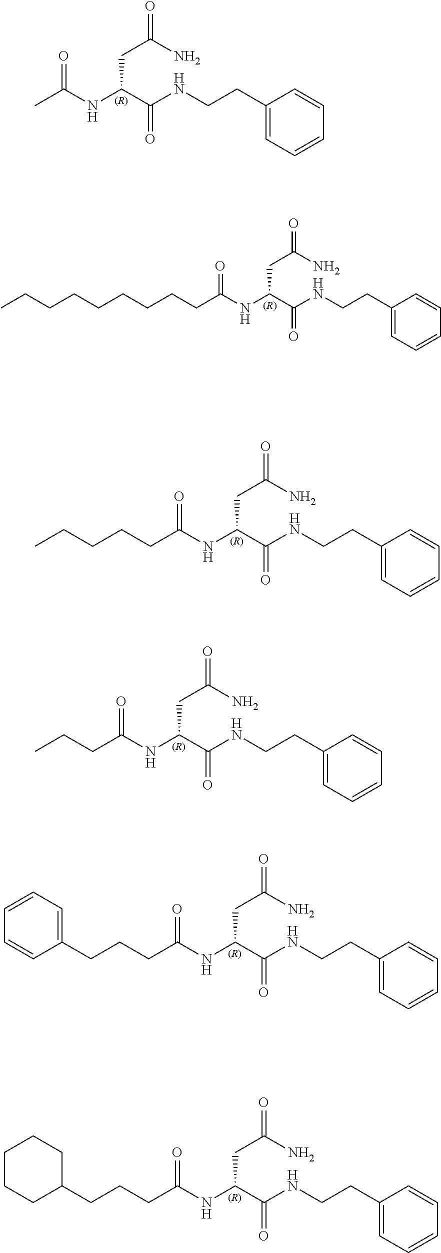

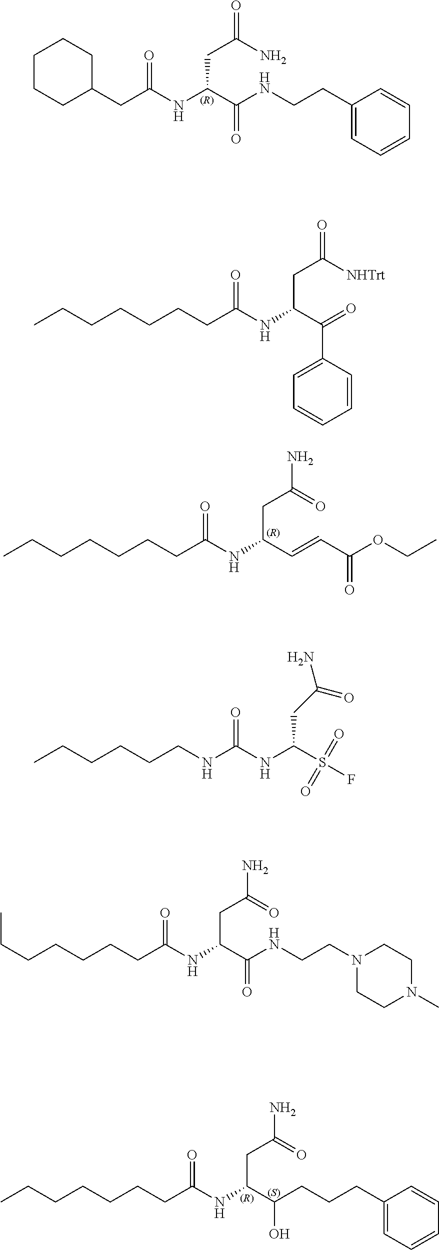

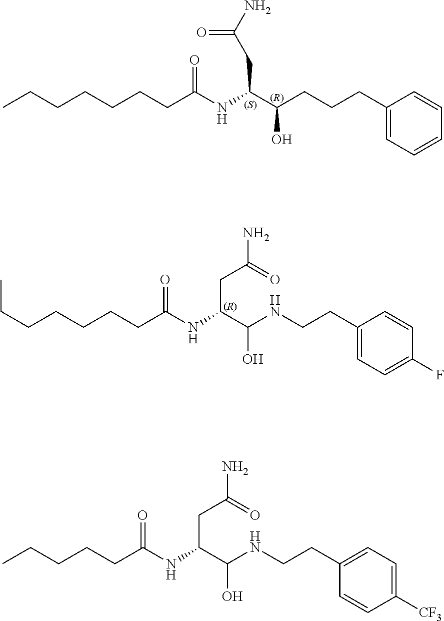

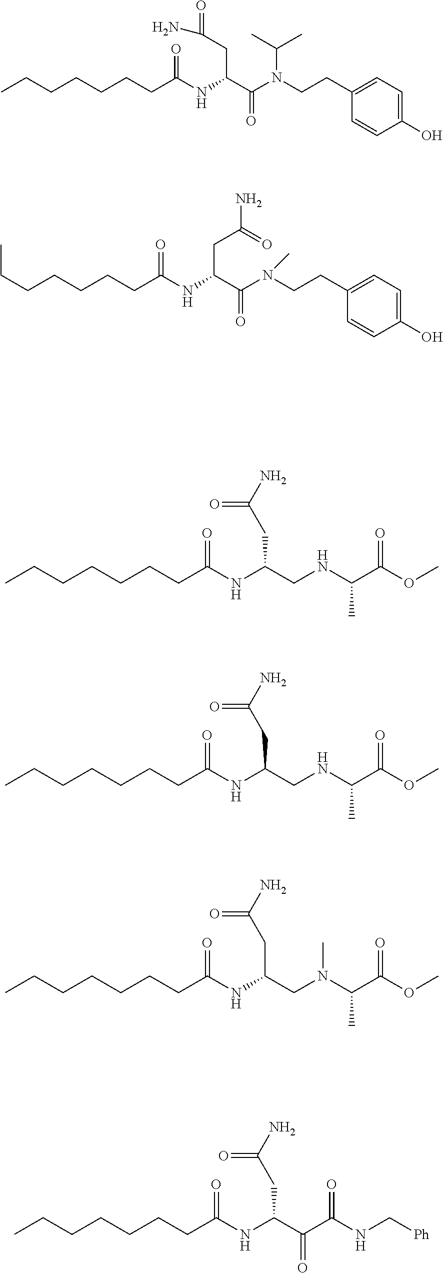

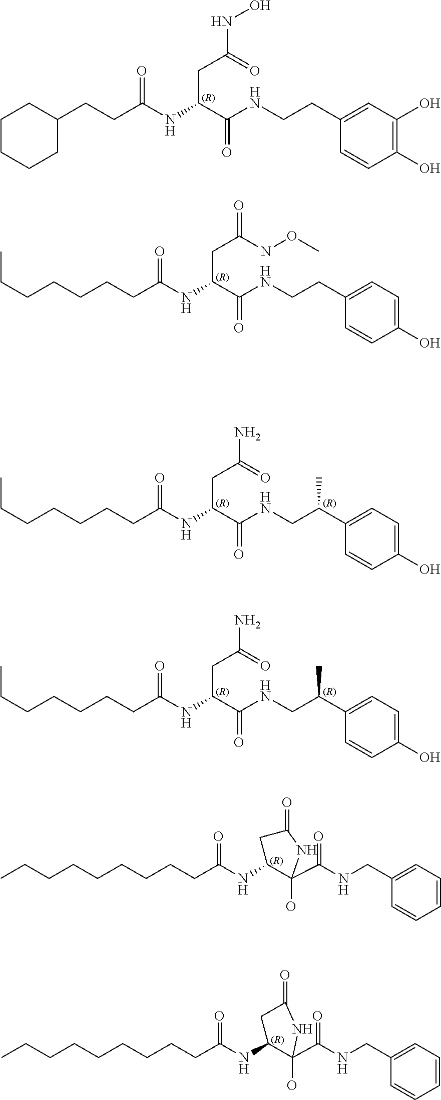

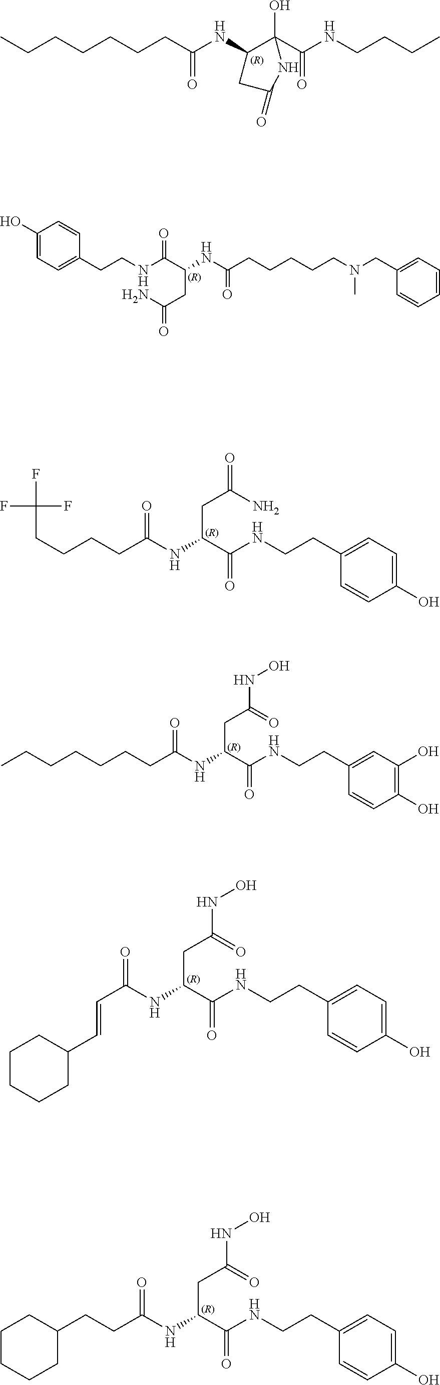

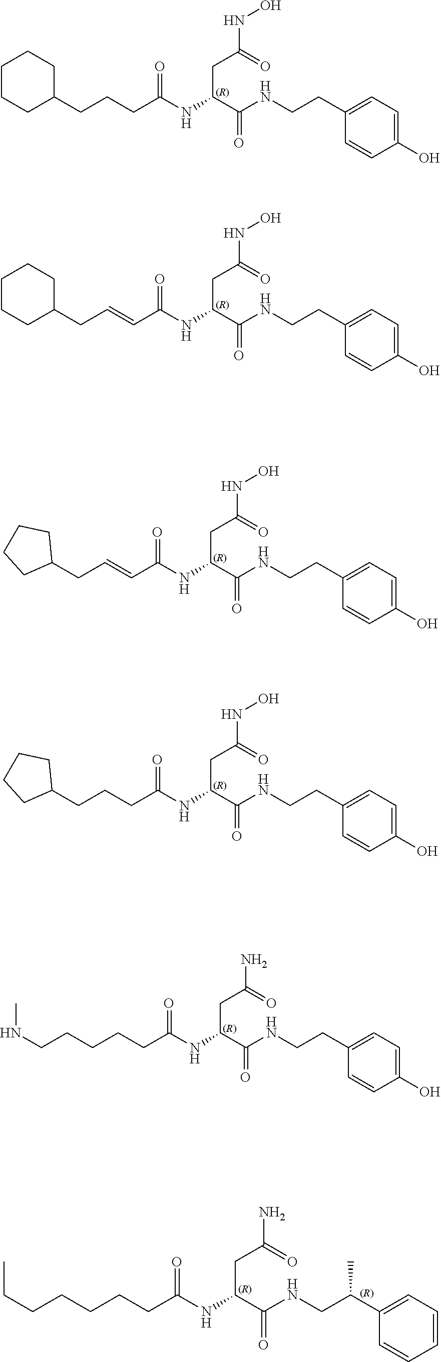

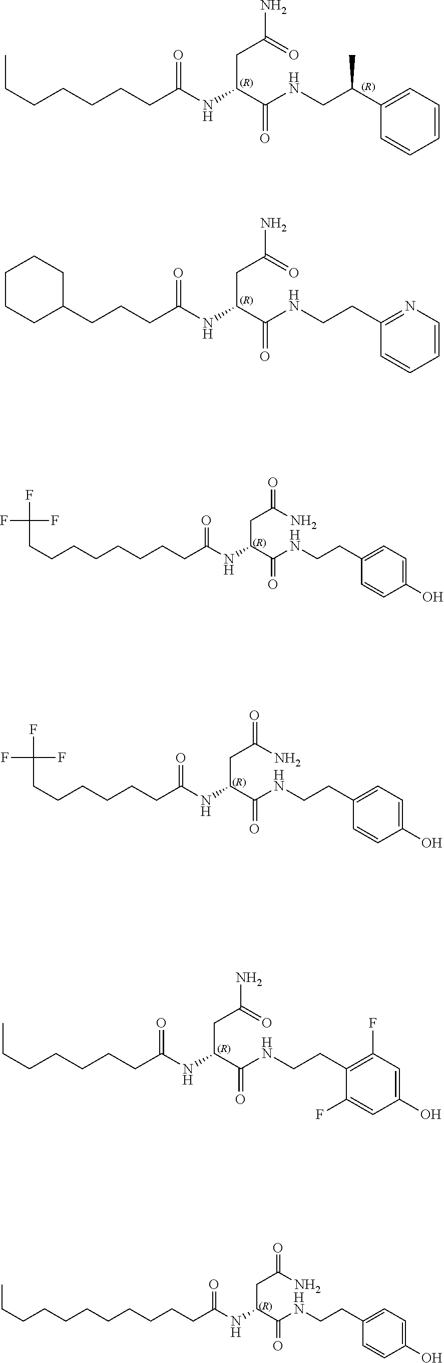

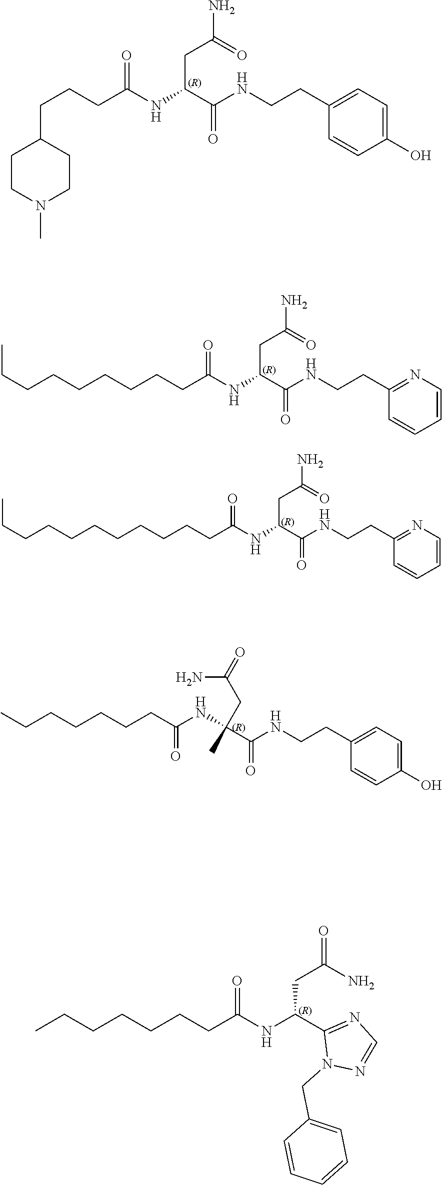

pharmaceutically acceptable salts thereof, and solvates of any of the foregoing.
Embodiment 6. A compound of Formula (I):

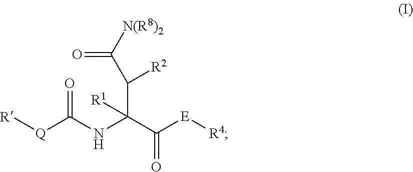

a pharmaceutically acceptable salt thereof, or a solvate of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
E is chosen from CR³R³′, NR³, and O, wherein optionally R¹ and R³ or R² and R³ together form a ring;
R³ and R^{3′} each independently is chosen from H, halogen, and C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.
   Embodiment 7. The compound of embodiment 6, wherein R′ is heptyl.
   Embodiment 8. The compound of embodiment 6 or 7, wherein Q is NH.
   Embodiment 9. The compound of embodiment 6 or 7, wherein Q is CH₂.
   Embodiment 10. A compound of Formula (II):

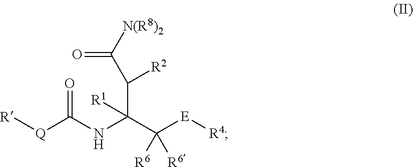

a pharmaceutically acceptable salt thereof, or a solvate of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
E is chosen from CR³R³′, NR³, and O, wherein optionally R¹ and R³ or R² and R³ together form a ring;
R³ and R^{3′} each independently is chosen from H, halogen, or C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl, and O-aryl; wherein the C₁₋₇ alkyl, C₁₋₆ alkyl-R⁵, C(═O)—C₀₋₆ alkyl-R⁵, O-alkyl or O-aryl is optionally substituted;
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted; and
R⁶ and R^{6′} each independently is chosen from H, OH, haloalkyl, and C₁₋₃ alkyl, or R⁶ and R^{6′} together form a 4-6 membered ring.
   Embodiment 11. The compound of embodiment 10, wherein R′ is heptyl.
   Embodiment 12. The compound of embodiment 10 or 11, wherein Q is NH.
   Embodiment 13. The compound of embodiment 10 or 11, wherein Q is CH₂.
   Embodiment 14. A compound of Formula (III):

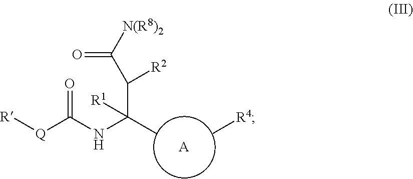

a pharmaceutically acceptable salt thereof, or a solvate of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
Ring A is chosen from 4-8 membered heteroaryl and 4-8 membered heterocyclyl; wherein the 4-8 membered heteroaryl or 4-8 membered heterocyclyl is optionally substituted;
R⁴ is chosen from C₁₋₇ alkyl, C₁₋₆ alkyl-R⁵, C(═O)—C₀₋₆ alkyl-R⁵, Cl, F, NH₂, O-alkyl, and O-aryl; wherein the C₁₋₇ alkyl, C₁₋₆ alkyl-R⁵, C(═O)—C₀₋₆ alkyl-R⁵, O-alkyl or O-aryl is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.
   Embodiment 15. The compound of embodiment 14, wherein R′ is heptyl.
   Embodiment 16. The compound of embodiment 14 or 15, wherein Q is NH.
   Embodiment 17. The compound of embodiment 14 or 15, wherein Q is CH₂.
   Embodiment 18. A compound of Formula (IV):

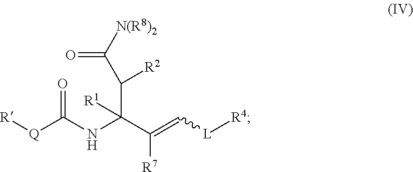

a pharmaceutically acceptable salt thereof, or a solvate of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl; L is chosen from S(═O)₂, C(═O), C(═O)NR³, and a bond;
R³ and R^{3′} each independently is chosen from H, halogen, and C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted;
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted; and
R⁷ is chosen from H, C₁₋₃ alkyl, and halogen.
   Embodiment 19. The compound of embodiment 18, wherein R′ is heptyl.
   Embodiment 20. The compound of embodiment 18 or 19, wherein Q is NH.
   Embodiment 21. The compound of embodiment 18 or 19, wherein Q is CH₂.
   Embodiment 22. A compound of Formula (V):

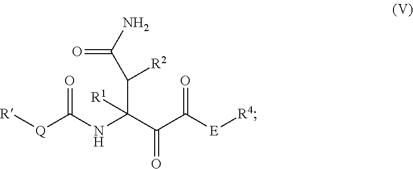

a pharmaceutically acceptable salt thereof, or a solvate of any of the foregoing,
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
E is chosen from CR³R³′, NR³, and O, wherein optionally R¹ and R³ or R² and R³ together form a ring;
R³ and R^{3′} each independently is chosen from H, halogen, and C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.
   Embodiment 23. The compound of embodiment 22, wherein R′ is heptyl.
   Embodiment 24. The compound of embodiment 22 or 23, wherein Q is NH.
   Embodiment 25. The compound of embodiment 22 or 23, wherein Q is CH₂.
   Embodiment 26. A compound of Formula (VI):

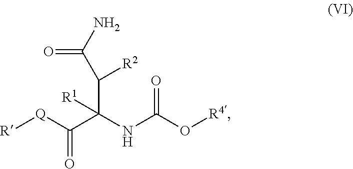

a pharmaceutically acceptable salt thereof, or a solvate of any of the foregoing, pharmaceutically acceptable salts thereof, and solvates of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
R^{4′} is chosen from C₁₋₇ alkyl and C₀₋₆ alkyl-R⁵; wherein the C₁₋₇ alkyl or C₀₋₆ alkyl-R⁵ is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.
   Embodiment 27. The compound of embodiment 26, wherein R′ is heptyl.
   Embodiment 28. The compound of embodiment 26 or 27, wherein Q is NH.
   Embodiment 29. The compound of embodiment 26 or 27, wherein Q is CH₂.
   Embodiment 30. A pharmaceutical composition comprising at least one pharmaceutically acceptable excipient and at least one entity chosen from compounds of any one of embodiments 1 to 29, pharmaceutically acceptable salts thereof, and solvates of any of the foregoing.
   Embodiment 31. A method of treating a disease in a subject in need thereof, the method comprising administering to the subject a therapeutically effective amount of the at least one entity chosen from compounds of any one of embodiments 1 to 29, pharmaceutically acceptable salts thereof, and solvates of any of the foregoing or a pharmaceutical composition of embodiment 30.
   Embodiment 32. A method of treating a disease or condition associated with colibactin expression, the method comprising administering to the subject a therapeutically effective amount of the at least one entity chosen from compounds of any one of embodiments 1 to 29, pharmaceutically acceptable salts thereof, and solvates of any of the foregoing or a pharmaceutical composition of embodiment 30.
   Embodiment 33. The method of embodiment 31 or 32, wherein the disease is cancer or familial adenomatous polyposis (FAP).
   Embodiment 34. The method of embodiment 33, wherein the cancer is colorectal cancer.
   Embodiment 35. A method of modulating a cancer marker in a subject in need thereof, the method comprising administering to the subject a therapeutically effective amount of the at least one entity chosen from compounds of any one of embodiments 1 to 29, pharmaceutically acceptable salts thereof, and solvates of any of the foregoing.
   Embodiment 36. The method of embodiment 35, wherein the cancer marker is a colorectal cancer marker selected from carbohydrate antigen 19-9 and/or carcinoembryonic antigen levels in the subject's blood, plasma, serum, stool, or urine.
   Embodiment 37. A method of treating colorectal cancer in a patient in need thereof, comprising:
(a) determining from a sample that the patient has a detectable level of a colibactin-adenine adduct or a metabolic derivative thereof, and
(b) administering an inhibitor of colibactin synthesis to the patient.
   Embodiment 38. A method of treating a colorectal cancer patient in need thereof, comprising:
(a) obtaining a biological sample from the patient;
(b) determining from the sample that the patient has a detectable level of a colibactin-adenine adduct or a metabolic derivative thereof; and
(c) administering an inhibitor of colibactin synthesis to the patient.
   Embodiment 39. The method of embodiment 38, wherein the biological sample comprises a plasma sample, a urine sample, a stool sample, an intestinal sample, or a combination thereof.
   Embodiment 40. The method of any one of embodiments 37 to 39, wherein the inhibitor of colibactin synthesis is a ClbP inhibitor or an antibiotic.

### EXAMPLES

The following non-limiting examples and data illustrate various aspects and features relating to the compounds and/or methods of the present disclosure, including the preparation of various compounds, as are available through the synthetic methodologies described herein. In comparison with the prior art, in some embodiments, the present compounds and/or methods provide results and data which are surprising, unexpected and contrary thereto. While the utility of this disclosure is illustrated through the use of several compounds and moieties/groups which can be used therewith, it will be understood by those skilled in the art that comparable results are obtainable with various other compounds, moieties and/or groups, as are commensurate with the scope of this disclosure.

### Preparation of Intermediates

### Example A: (R)-4-amino-1,4-dioxo-1-(phenethylamino)butan-2-aminium trifluoacetate

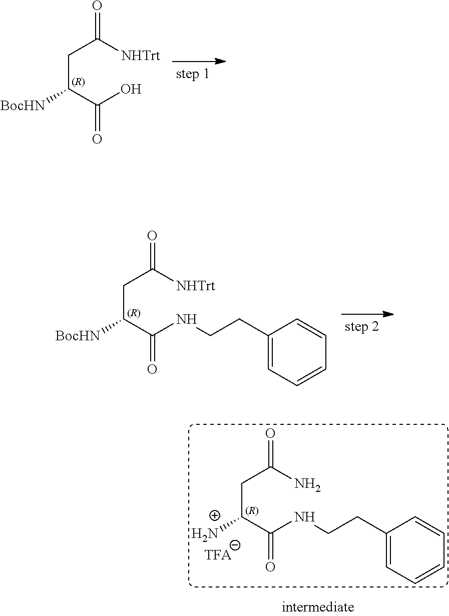

### Step 1: Synthesis of tert-butyl N-[(1R)-1-[(2-phenylethyl)carbamoyl]-2-[(triphenylmethyl) carbamoyl]ethyl]carbamate

To a solution of Boc-D-Asn(Trt)-OH (5.0 g, 10.5 mmol, 1.0 eq) and phenethylamine (1.53 g, 12.6 mmol, 1.2 eq) in anhydrous DMF (75 ml) was added HATU (4.81 g, 12.6 mmol, 1.2 eq) and DIPEA (3.00 g, 23.2 mmol, 2.2 eq). After 12 h, the LCMS analysis confirmed the formation of desired product. Then water was added to the reaction mixture and the precipitate was filtered, washed with water and dried under reduced pressure to give tert-butyl N-[(1R)-1-[(2-phenylethyl)carbamoyl]-2-[(triphenylmethyl)carbamoyl]ethyl]carbamate (6.00 g, 79%) as an yellow solid.

¹H NMR (300 MHz, Chloroform-d): δ 7.36-7.10 (m, 20H), 7.01 (s, 1H), 6.74 (s, 1H), 6.18 (d, J=8.0 Hz, 1H), 4.44 (s, 1H), 3.47 (dp, J=19.7, 6.4 Hz, 2H), 3.11 (dd, J=15.1, 4.0 Hz, 1H), 2.75 (t, J=7.3 Hz, 2H), 2.55 (dd, J=15.0, 5.8 Hz, 1H), 1.41 (s, 9H).

### Step 2: Synthesis of (1R)-2-carbamoyl-1-[(2-phenylethyl)carbamoyl]ethan-1-aminium trifluoroacetate

To a solution of tert-butyl N-[(1R)-1-[(2-phenylethyl)carbamoyl]-2-[(triphenylmethyl)carbamoyl]ethyl]carbamate (7.5 g, 10.4 mmol, 1.0 eq) in dichloromethane (9 ml) was added water (2 mL, 10.0 eq), triethylsilane (3.32 ml, 20.8 mmol, 2.0 eq) and finally trifluoroacetic acid (39.7 ml, 520 mmol, 50.0 eq). After 12 h, LC-MS analysis confirmed formation of the desired product. Then water was added to the reaction mixture and the aqueous phase was washed with dichloromethane. After that, the aqueous layer was dried under reduced pressure to give (1R)-2-carbamoyl-1-[(2-phenylethyl)carbamoyl]ethan-1-aminium trifluoroacetate (3.5 g, 87%) as a white solid.

¹H NMR (300 MHz, Methanol-d₄): δ 7.35-7.28 (m, 2H), 7.26-7.19 (m, 3H), 4.12 (dd, J=8.1, 5.2 Hz, 1H), 3.63-3.37 (m, 2H), 2.87-2.80 (m, 2H), 2.77-2.72 (m, 2H).

### Example B: (R)

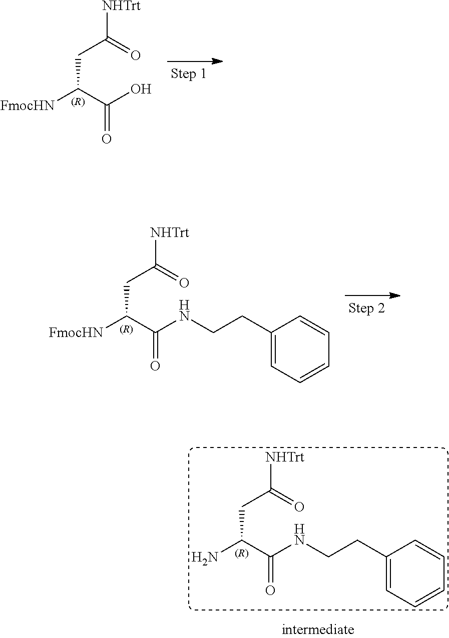

### Step 1: Synthesis of 9H-fluoren-9-ylmethyl N-[(1R)-1-[(2-phenylethyl)carbamoyl]-2-[(triphenylmethyl)carbamoyl]ethyl]carbamate

To a solution of Fmoc-D-Asn(Trt)-OH (2.5 g, 4.19 mmol, 1.0 eq) and phenethylamine (0.50 g, 4.19 mmol, 1.0 eq) in anhydrous ACN (75 ml) HBTU (1.90 g, 5.03 mmol, 1.2 eq) and N,N-diisopropylethylamine (0.59 g, 4.61 mmol, 1.1 eq) were added. After 12 h, the LCMS analysis confirmed the formation of desired product. The solvent was evaporated and the precipitate was filtered, washed with water, acetonitrile, and dried under reduced pressure to give 9H-fluoren-9-ylmethyl N-[(1R)-1-[(2-phenylethyl)carbamoyl]-2-[(triphenylmethyl)-carbamoyl]ethyl]carbamate (1.5 g, 50%) as a white solid.

### Step 2: Synthesis of (2R)-2-amino-N-(2-phenylethyl)-N′-(triphenylmethyl)butanediamide

To a solution of N-[(1R)-1-[(2-phenylethyl)carbamoyl]-2-[(triphenylmethyl)carbamoyl]-ethyl]carbamate (1.5 g, 2.14 mmol, 1.0 eq) in anhydrous ACN (75 mL) piperidine (1.50 g, 12.2 mmol, 6.0 eq) was added. After 1 h, the LCMS analysis confirmed the formation of desired product. The solvent was evaporated under reduced pressure and residue was washed with hot hexane and filtered to give (2R)-2-amino-N-(2-phenylethyl)-N′-(triphenylmethyl)butanediamide (0.90 g, 87%) as a yellow oil. Crude product was used in the next step.

### Example 00: Methyl octanoyl-D-asparaginyl-L-alaninate

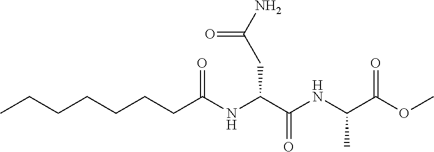 [SMILES: CCCCCCCC(=O)N[C@H](CC(N)=O)C(=O)N[C@@H](C)C(=O)OC]

### Step 1

To a solution of (tert-butoxycarbonyl)-D-asparagine (998 mg, 4.30 mmol, 1.2 equiv.), HOBt (532 mg, 3.94 mmol, 1.1 equiv.), methyl L-alaninate·HCl (500 mg, 3.58 mmol, 1 equiv.), TEA (797 mg, 7.88 mmol, 1.10 mL, 2.2 equiv.) in DMF (5 mL) was added EDCI (755 mg, 3.94 mmol, 1.1 equiv.) at 0° C. The mixture was stirred at 25° C. for 12 hr. The reaction mixture was filtered and concentrated under reduced pressure and the residue was purified by prep-HPLC (column: Phenomenex luna C18 250*50 mm*10 um; mobile phase: [water(0.1% TFA)-ACN]; B %: 5%-30%, 20 min) to give methyl (tert-butoxycarbonyl)-D-asparaginyl-L-alaninate (1 g, 88%) as a white solid.

### Step 2

To a solution of methyl (tert-butoxycarbonyl)-D-asparaginyl-L-alaninate (300 mg, 945 μmol) in EtOAc (3 mL) was added HCl/EtOAc (3 mL) (4M) at 25° C. The mixture was stirred at 25° C. for 2 hr. The reaction mixture was concentrated under reduced pressure to give methyl (tert-butoxycarbonyl)-D-asparaginyl-L-alaninate hydrochloride (100 mg, 394.19 μmol, 42%) as a yellow solid.

### Step 3

To a solution of methyl (tert-butoxycarbonyl)-D-asparaginyl-L-alaninate hydrochloride (100 mg, 394 μmol 1 equiv.), octanoic acid (68 mg, 473 μmol, 75 μL, 1.2 equiv.) and HOBt (59 mg, 434 μmol, 1.1 equiv.) in DMF (5 mL) was added TEA (88 mg, 867 μmol, 121 μL, 2.2 equiv.) at 25° C. The mixture was stirred at 25° C. for 5 min. The reaction mixture was cooled to 0° C. and EDCI (83 mg, 434 μmol, 1.1 equiv.) was added. The mixture was stirred at 0° C. for 15 min and was then warmed to 25° C. and stirred at 25° C. for 12 hr. The reaction mixture was filtered and concentrated under reduced pressure and the residue was purified by prep-HPLC (column: Nano-micro Kromasil C18 100*30 mm 5 um; mobile phase: [water(0.1% TFA)-ACN]; B %: 18%-48%, 10 min) to give the title compound (55 mg, 40%) as a white solid. LCMS: (M+H⁺) 344.2 ¹H NMR (400 MHz, DMSO-d₆) δ 8.08 (d, J=7.3 Hz, 1H), 7.94 (d, J=8.2 Hz, 1H), 7.27 (s, 1H), 6.87 (s, 1H), 4.59 (q, J=7.7 Hz, 1H), 4.25 (p, J=7.2 Hz, 1H), 3.62 (s, 3H), 2.50-2.44 (m, 1H), 2.33 (dd, J=15.3, 7.8 Hz, 1H), 2.10 (t, J=7.5 Hz, 2H), 1.48 (t, J=7.2 Hz, 2H), 1.29-1.22 (m, 11H), 0.86 (t, J=6.6 Hz, 3H).

### Example 01: N-((3R)-5-amino-2-(ethylamino)-1,1,1-trifluoro-5-oxopentan-3-yl)octanamide

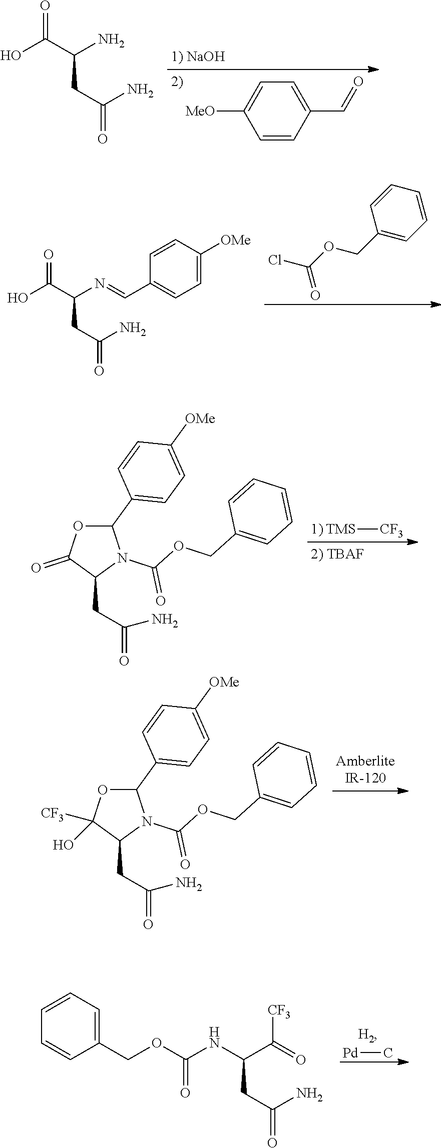

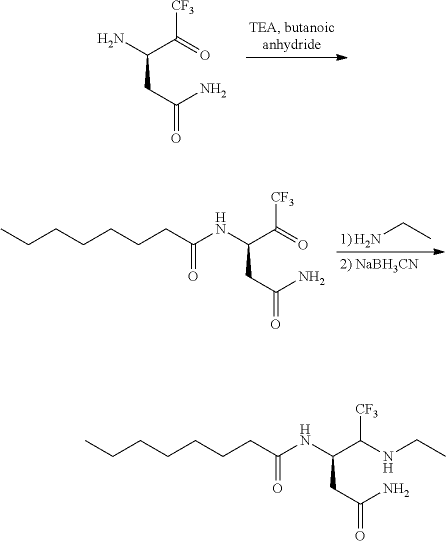

### Steps 1-3

D-asparagine dissolved in a 1 M solution of sodium hydroxide is treated with 1 mol equiv of aldehyde. Concentration in vacuo is continued until the reaction mixture solidified and the solid is filtered, washed thoroughly with same solvent and dried in vacuo to yield the Schiff base salts. To a suspension of carefully ground Schiff base salt in DCM is added benzyl chloroformate slowly by syringe pump under an argon atmosphere. Upon reaction completion the mixture is reduced in vacuo and extracted with ethyl acetate. The solvents are removed in vacuo and the oxazolidinone is purified by column chromatography from suitable solvents.

Steps 4-6: To a solution of oxazolidin-5-one (1 mM) in dry THF is added cesium fluoride (catalytic amounts) and (trifluoromethyl)trimethylsilane (1.2 equiv) under an argon atmosphere. After all starting material is consumed the mixture is extracted with ethyl acetate, purified by flash column chromatography to produce the oxazolidine. The oxazolidine is treated with 1.20 equiv of TBAF in THF. Upon consumption of starting materials the trifluoromethyl ketone is extracted with ethyl acetate and volatiles removed to produce desilylated oxazolidine. The product is then subjected to strongly acidic cation-exchange resin Amberlite IR-120. After completion of hydrolysis the solution is filtered through Celite with acetonitrile, extracted with ethyl acetate, and the trifluoromethyl ketone is purified by column chromatography from suitable solvents.

Steps 7-10: To a solution of trifluoromethyl ketone (1 mM) in ethanol is added palladium on carbon catalyst and hydrochloric acid (1 M solution). The mixture is placed under a hydrogen balloon and stirred until reaction is completed. The catalyst is filtered off through a plug of Celite. Following evaporation of volatiles the crude amino trifluoro ketone is taken up in aqueous hydrochloric acid which is extracted with ethyl acetate. The hydrochloride salt is isolated following evaporation of volatiles. The salt is then treated with butanoic anhydride and triethylamine. Following work-up the trifluoromethyl ketone is purified by column chromatography from suitable solvents. The resultant trifluoromethyl ketone is treated with ethylamine to form trifluoromethyl imine. The solution is then treated with sodium cyanoborohydride. Upon completion of reduction the reaction is worked up and then purified via column chromatography from suitable solvents to produce the desired trifluoromethylamine.

Synthesis adapted from Walter et al., (1998) J. Org. Chem. 63, 5179-5192.

### Example 02: N-((3R)-5-amino-2-(tert-butylamino)-1,1,1-trifluoro-5-oxopentan-3-yl)octanamide

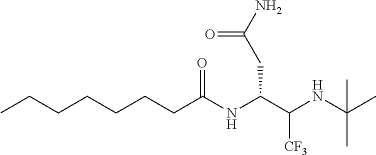 [SMILES: CCCCCCCC(=O)N[C@H](CC(N)=O)C(NC(C)(C)C)C(F)(F)F]

To be prepared according to procedure for Example 01, replacing ethyl amine in step 9, with tert-butylamine.

### Example 03: N-((3R)-5-amino-1,1,1-trifluoro-5-oxo-2-(phenethylamino)pentan-3-yl)octanamide

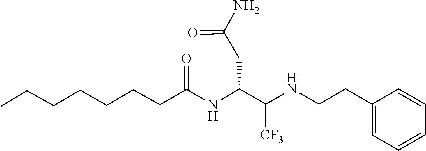 [SMILES: CCCCCCCC(=O)N[C@H](CC(N)=O)C(NCCc1ccccc1)C(F)(F)F]

To be prepared according to procedure for Example 01, replacing ethyl amine in step 9, with phenethylamine.

### Example 04: (3R)-5,5,5-trifluoro-3-(3-hexylureido)-4-(phenethylamino)pentanamide

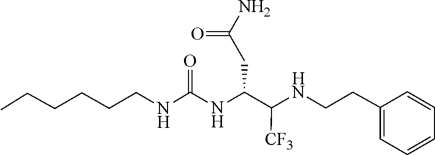 [SMILES: CCCCCCNC(=O)N[C@H](CC(N)=O)C(NCCc1ccccc1)C(F)(F)F]

To be prepared according to procedure for Example 01, replacing octanoic anhydride with 1-isocyanatohexane.

### Example 05: hexyl ((3R)-5-amino-1,1,1-trifluoro-5-oxo-2-(phenethylamino)pentan-3-yl)carbamate

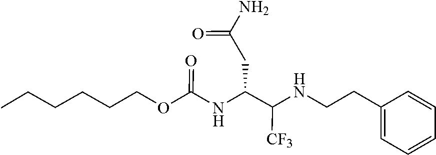 [SMILES: CCCCCCOC(=O)N[C@H](CC(N)=O)C(NCCc1ccccc1)C(F)(F)F]

To be prepared according to procedure for Example 01, replacing octanoic anhydride with hexylchloroformate.

### Example 06: (R)-N-(3-amino-1-(3-(ethylamino)oxetan-3-yl)-3-oxopropyl)octanamide

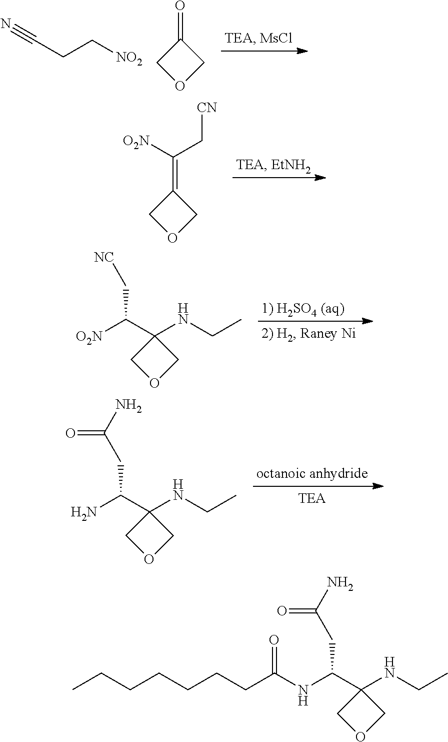

Compound 3-nitropropanenitrile (1 equiv) is stirred with oxetan-3-one (1.1 equiv), triethylamine (1.1 equiv), and methanesulfonyl chloride (1.1 equiv), followed by reaction with triethylamine (1.1 equiv) and ethylamine (1.1 equiv). The resulting product is stirred in aqueous sulfuric acid, and then treated with H₂ over Raney Ni. The amine is treated with octanoic anhydride (1.1 equiv) and triethylamine (1.1 equiv), then purified by reverse-phase C18 column chromatography to afford the title compound.

### Example 07: (R)-N-(3-amino-3-oxo-1-(3-(phenethylamino)oxetan-3-yl)propyl)octanamide

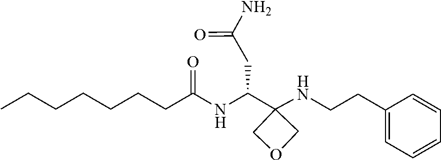 [SMILES: CCCCCCCC(=O)N[C@H](CC(N)=O)C1(NCCc2ccccc2)COC1]

To be prepared according to procedure for Example 06, replacing ethylamine with phenethylamine in step 2.

### Example 08: (R)-N-(3-amino-1-(3-(tert-butylamino)oxetan-3-yl)-3-oxopropyl)octanamide

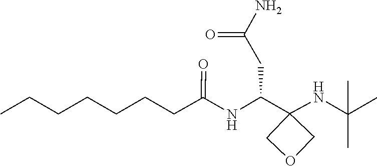 [SMILES: CCCCCCCC(=O)N[C@H](CC(N)=O)C1(NC(C)(C)C)COC1]

To be prepared according to procedure for Example 06, replacing ethylamine with tert-butylamine in step 2.

### Example 09: (R)-3-(3-hexylureido)-3-(3-(phenethylamino)oxetan-3-yl)propenamide

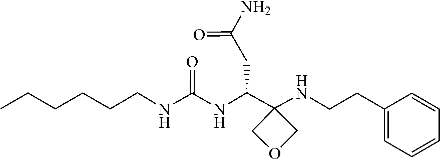 [SMILES: CCCCCCNC(=O)N[C@H](CC(N)=O)C1(NCCc2ccccc2)COC1]

To be prepared according to procedure or Example 6, replacing octanoic anhydride with 1-isocyanatohexane.

### Example 10: hexyl (R)-(3-amino-3-oxo-1-(3-(phenethylamino)oxetan-3-yl)propyl)carbamate

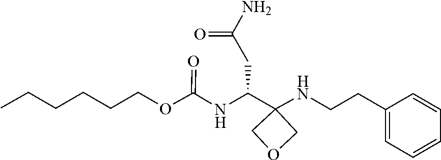 [SMILES: CCCCCCOC(=O)N[C@H](CC(N)=O)C1(NCCc2ccccc2)COC1]

To be prepared according to procedure for Example 06, replacing octanoic anhydride with hexylchloroformate

### Example 11: (R)-N1-(tert-butyl)-2-octanamidosuccinamide

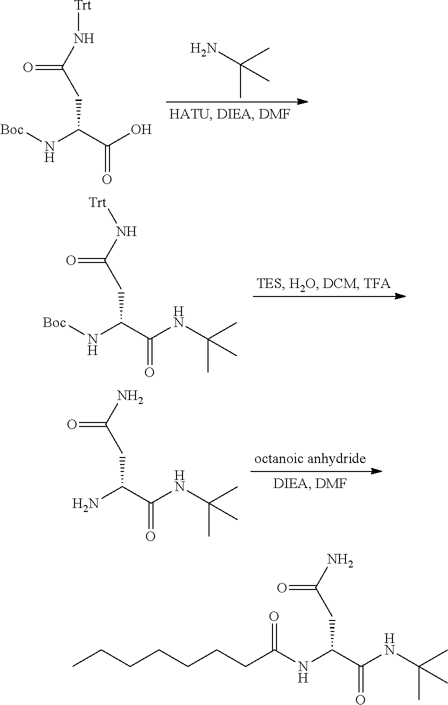

Nα-Boc-Nγ-trityl-D-asparagine (1 equiv) was treated with HATU (1.05 equiv), DIPEA (2.1 equiv), and tert-butylamine (1.1 equiv) in DMF overnight at room temperature, followed by treatment with triethylsilane, water, dichloromethane, and TFA (10 equiv). The resulting compound was stirred in octanoic anhydride (1.05 equiv) and N,N-diisopropylethylamine (1.1 equiv) in DMF, then purified by reverse phase C18 column chromatography to yield the title compound.

¹H NMR (400 MHz, DMSO-d₆) δ 7.92 (d, J=8.0 Hz, 1H), 7.28-7.18 (m, 2H), 6.85 (bs), 4.47 (ddd, J=8.1, 7.2, 6.6 Hz, 1H), 2.44 (dd, J=15.2, 6.3 Hz, 1H), 2.28 (dd, J=15.3, 7.6 Hz, 1H), 2.08 (t, J=7.4, 2H), 1.51-1.40 (m, 2H), 1.30-1.15 (m, 17H), 0.85 (t, J=6.7 Hz, 3H).

### Example 12A: (R)-2-octanamido-N1-phenethylsuccinamide

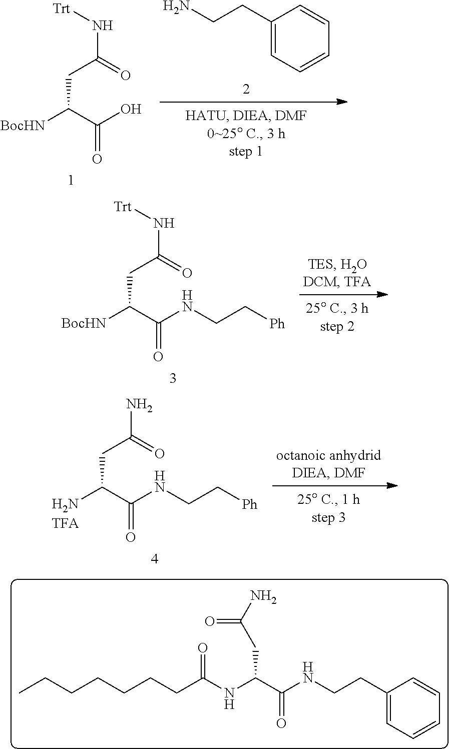

### Step 1

To a solution of (2S)-2-(tert-butoxycarbonylamino)-4-oxo-4-(tritylamino)butanoic acid (500 mg, 1.05 mmol, 1 eq) and 2-phenylethanamine (153.21 mg, 1.26 mmol, 158.77 uL, 1.2 eq) in DMF (15 mL) was added HATU (480.75 mg, 1.26 mmol, 1.2 eq) and DIPEA (299.59 mg, 2.32 mmol, 403.75 uL, 2.2 eq) at 0° C. The mixture was stirred at 25° C. for 3 hr. LCMS showed the starting reactant was consumed and have the desired mass. The reaction mixture was quenched by addition H2O 30 mL at 0° C., and the precipitate was filtered and filter cake was concentrated to give compound tert-butyl N-[(1R)-3-oxo-1-(2-phenylethylcarbamoyl)-3-(tritylamino)propyl]carbamate (500 mg, 778.94 umol, 73.93% yield, 90% purity) as a yellow solid.

### Step 2

The mixture of tert-butyl N-[(1R)-3-oxo-1-(2-phenylethylcarbamoyl)-3-(tritylamino)propyl]carbamate (400 mg, 692.39 umol, 1 eq) in TES (0.25 mL), H2O (0.25 mL), DCM (0.5 mL) and TFA (9 mL) was stirred at 25° C. for 3 hr. LCMS showed the desired mass. The mixture was concentrated to give compound (2R)-2-amino-N-(2-phenylethyl)butanediamide (250 mg, crude, TFA) as a yellow solid.

### Step 3

To a solution of (2R)-2-amino-N-(2-phenylethyl)butanediamide (250 mg, 715.71 umol, 1 eq, TFA) and octanoyl octanoate (290.30 mg, 1.07 mmol, 1.5 eq) in DMF (5 mL) was added DIPEA (277.50 mg, 2.15 mmol, 373.99 uL, 3 eq). The mixture was stirred at 25° C. for 1 hr. LCMS showed the starting reactant was consumed and the desired mass was detected. The mixture was concentrated to give a residue. The residue was purified by prep-HPLC (column: Phenomenex Luna C18 100*30 mm*5 um; mobile phase: [water(0.1% TFA)-ACN]; B %: 30%-55%, 10 min) to give compound (2R)-2-(octanoylamino)-N-(2-phenylethyl)butanediamide (131 mg, 362.40 umol, 50.64% yield, 100% purity) as a white solid.

¹H NMR (400 MHz, DMSO-d₆) δ 7.88 (d, J=8.1 Hz, 1H), 7.74 (t, J=5.8 Hz, 1H), 7.31-7.25 (m, 2H), 7.24-7.16 (m, 4H), 6.81 (bs, 1H), 4.49 (ddd, J=8.3, 7.3, 6.1 Hz, 1H), 3.26 (m, 2H), 2.68 (t, 7.4 Hz, 2H), 2.45 (dd, J=15.3, 5.9 Hz, 1H), 2.31 (dd, J=15.1, 7.8 Hz, 1H), 2.08 (t, J=7.5, 2H), 1.51-1.40 (m, 2H), 1.32-1.17 (m, 8H), 0.85 (t, J=6.6 Hz, 3H). LCMS (ESI, m/z): (M+H)⁺ calcd for C₂₀H₃₂N₃O₃, 362.2; found, 362.2.

### Example 12B: (S)-2-octanamido-N1-phenethylsuccinamide

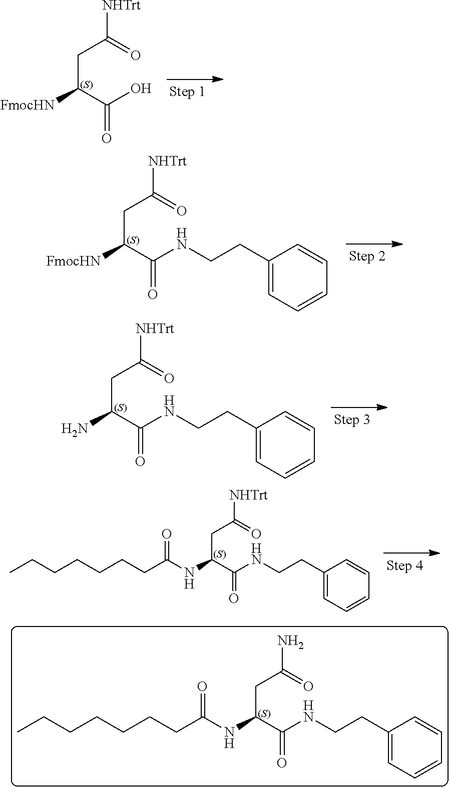

Step 1: Synthesis of (9H-fluoren-9-yl)methyl N-[(1S)-1-[(2-phenylethyl)carbamoyl]-2-[(triphenylmethyl)carbamoyl]ethyl]carbamate

To a solution of Fmoc-L-Asn(Trt)-OH (0.500 g, 0.84 mmol, 1.0 eq) and phenethylamine (0.106 mL, 0.84 mmol, 1.0 eq) in anhydrous ACN (100 ml) HBTU (0.38 g, 1.01 mmol, 1.2 eq) and N,N-diisopropylethylamine (0.161 mL, 0.92 mmol, 1.1 eq) were added. After 16 h, the LCMS analysis confirmed the formation of desired product. The solvent was evaporated and the precipitate was filtered, washed with water, acetonitrile, and dried under reduced pressure to give (9H-fluoren-9-yl)methyl N-[(1S)-1-[(2-phenylethyl)carbamoyl]-2-[(triphenylmethyl)carbamoyl]ethyl]carbamate (0.30 g, 50%) as a white solid.

### Step 2: Synthesis of (2S)-2-amino-N-(2-phenylethyl)-N′-(triphenylmethyl)butanediamide

To a solution of (9H-fluoren-9-yl)methyl N-[(1S)-1-[(2-phenylethyl)carbamoyl]-2-[(triphenylmethyl)carbamoyl]ethyl]carbamate (0.29 g, 0.41 mmol, 1.0 eq) in anhydrous ACN (20 mL) piperidine (0.25 mL, 0.25 mmol, 6.0 eq) was added. After 2 h, the LCMS analysis confirmed formation of the desired product. The solvent was evaporated under reduced pressure, the residue was washed with hot hexane and filtered to give (2S)-2-amino-N-(2-phenylethyl)-N′-(triphenylmethyl)butanediamide (0.16 g, 81%) as an yellow oil. The crude product was used in the next step.

### Step 3: Synthesis of (2S)-2-octanamido-N-(2-phenylethyl)-N′-(triphenylmethyl)butanediamide

To a solution of (2S)-2-amino-N-(2-phenylethyl)-N′-(triphenylmethyl)butanediamide (0.15 g, 0.32 mmol, 1.0 eq) and octanoic acid (0.051 mL, 0.32 mmol, 1.0 eq) in anhydrous ACN (5 ml) was added HBTU (0.15 g, 0.39 mmol, 1.2 eq) and N,N-diisopropylethylamine (0.062 mL, 0.36 mmol, 1.1 eq) and reaction mixture was stirred at room temperature for 16 h. The LCMS analysis confirmed the formation of desired product. The precipitate was filtered, washed with water, acetonitrile, and dried under reduced pressure to give (2S)-2-octanamido-N-(2-phenylethyl)-N′-(triphenylmethyl)butanediamide (0.12 g, 61%) as a white solid.

### Step 4: Synthesis of (2S)-2-octanamido-N-(2-phenylethyl)butanediamide

To a solution of (2S)-2-octanamido-N-(2-phenylethyl)-N′-(triphenylmethyl)butanediamide (0.12 g, 0.20 mmol, 1.0 eq) in dichloromethane (10 ml) was added water (0.1 mL, 10 eq), triethylsilane (0.063 ml, 0.40 mmol, 2.0 eq) and finally trifluoroacetic acid (0.76 ml, 10 mmol, 50 eq). The reaction mixture was stirred at room temperature for 2 h. The LC-MS analysis confirmed formation of the desired product. Water was added and the aqueous phase was washed with dichloromethane. After that, the aqueous layer was dried under reduced pressure and further purified by flash column chromatography (DCM:MeOH, 9:1) to give (2S)-2-octanamido-N-(2-phenylethyl)butanediamide (0.030 g, 42%) as a white solid.

¹H NMR (400 MHz, DMSO-d₆) δ 7.91 (d, J=8.1 Hz, 1H), 7.78 (t, J=5.7 Hz, 1H), 7.34-7.16 (m, 5H), 6.89-6.80 (m, 1H), 4.50 (td, J=7.9, 6.0 Hz, 1H), 3.30-3.20 (m, 2H), 2.68 (t, J=7.4 Hz, 2H), 2.45 (dd, J=15.2, 5.9 Hz, 1H), 2.31 (dd, J=15.2, 7.7 Hz, 1H), 2.09 (t, J=7.5 Hz, 2H), 1.48 (q, J=7.2 Hz, 2H), 1.24 (s, 8H), 0.90-0.80 (m, 3H).

### Example 13: (R)-2-octanamido-N1-ethylsuccinamide

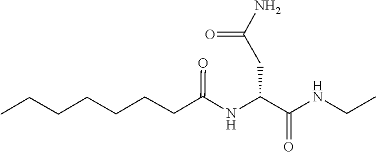 [SMILES: CCCCCCCC(=O)N[C@H](CC(N)=O)C(=O)NCC]

The title compound was synthesized according to the experimental procedure described for Example 11, replacing tert-butyl amine with ethylamine.

¹H NMR (400 MHz, DMSO-d₆) δ 7.88 (d, J=8.1 Hz, 1H), 7.74 (t, J=5.8 Hz, 1H), 7.23 (bs, 1H), 6.83 (bs, 1H), 4.47 (ddd, J=7.9, 7.5, 6.3 Hz, 1H), 3.03 (m, 2H), 2.45 (dd, J=15.3, 6.0 Hz, 1H), 2.31 (dd, J=15.2, 7.6 Hz, 1H), 2.08 (t, J=7.4, 2H), 1.51-1.41 (m, 2H), 1.31-1.13 (m, 8H), 0.97 (t, J=7.2 Hz, 3H), 0.85 (t, J=6.7 Hz, 3H). LCMS (ESI, m/z): (M+H)⁺ calcd for C₁₄H₂₈N₃O₃, 286.2; found, 286.1.

### Example 14: (R)-2-(3-hexylureido)-N1-phenethylsuccinamide

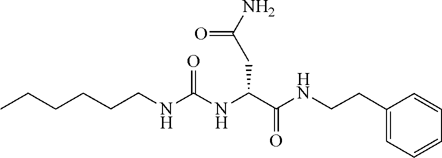 [SMILES: CCCCCCNC(=O)N[C@H](CC(N)=O)C(=O)NCCc1ccccc1]

The title compound may be synthesized according to the experimental procedure described for Example 11, replacing tert-butyl amine with phenethylamine and octanoic anhydride with hexyl isocyanate.

### Example 15: (R)-2-(3-hexylureido)-N1-phenethylsuccinamide

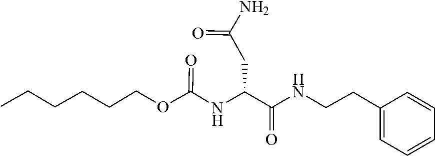 [SMILES: CCCCCCOC(=O)N[C@H](CC(N)=O)C(=O)NCCc1ccccc1]

The title compound may be synthesized according to the experimental procedure described for Example 11, replacing tert-butyl amine with phenethylamine and octanoic anhydride with hexyl chloroformate.

### Example 16: methyl N-methyl-N-(octanoyl-D-asparaginyl)-L-alaninate

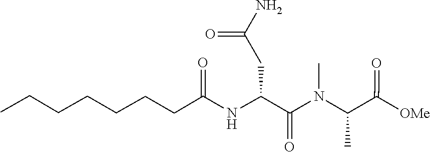 [SMILES: CCCCCCCC(=O)N[C@H](CC(N)=O)C(=O)N(C)[C@@H](C)C(=O)OC]

The title compound may be synthesized according to the experimental procedure described for Example 11, replacing tert-butyl amine with N-methyl-L-alanine methyl ester.

¹H NMR (400 MHz, DMSO-d₆, mixture of rotamers) δ 8.27 (d, J=8.7 Hz, 0.3H), 8.12 (d, J=8.9 Hz, 0.7H), 7.33-7.24 (m, 1H), 6.86-6.74 (m, 1H), 5.11-4.99 (m, 1H), 4.96-4.89 (m, 0.3H), 4.82-4.75 (m, 0.7H), 3.67 (s, 0.9H), 3.58 (s, 2.1H), 2.97 (s, 2.1H), 2.67 (s, 0.9H), 2.62-2.51 (m, 1.4H), 2.33 (m, 0.3H), 2.24-2.14 (m, 0.9H), 2.10-2.02 (m, 1.4H), 1.51-1.41 (m, 2H), 1.33 (d, J=6.9 Hz, 0.9H), 1.29-1.16 (m, 10.1H), 0.85 (t, J=6.7 Hz, 3H). LCMS (ESI, m/z): (M+H)⁺ calcd for C₁₇H₃₂N₃O₅, 358.2; found, 358.1.

### Example 17: (R)-N1-methyl-2-octanamido-N1-phenethylsuccinamide

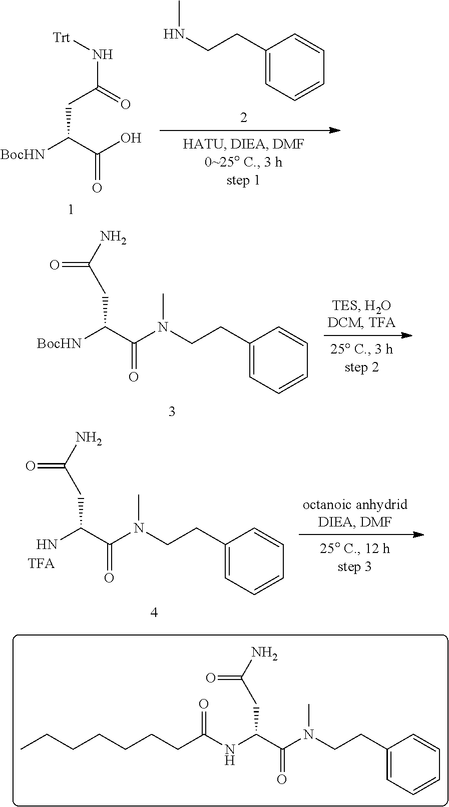

### Step 1

To a solution of (2R)-2-(tert-butoxycarbonylamino)-4-oxo-4-(tritylamino)butanoic acid (1 g, 2.11 mmol, 1 eq) and N-methyl-2-phenyl-ethanamine (341.90 mg, 2.53 mmol, 367.63 uL, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 3 hr. TLC indicated (2R)-2-(tert-butoxycarbonylamino)-4-oxo-4-(tritylamino)butanoic acid was consumed completely and one new spot formed. The reaction mixture was quenched by addition H₂O 30 mL at 0° C., and the precipitate was filtered and filter cake was concentrated under reduced pressure. Compound tert-butyl N-[(1R)-1-[methyl(2-phenylethyl)carbamoyl]-3-oxo-3-(tritylamino)propyl]carbamate (1.3 g, 2.09 mmol, 99.04% yield, 95% purity) was obtained as a white solid.

### Step 2

A mixture of tert-butyl N-[(1R)-1-[methyl(2-phenylethyl)carbamoyl]-3-oxo-3-(tritylamino)propyl]carbamate (1 g, 1.69 mmol, 1 eq) in TES (968.55 mg, 4.22 mmol, 0.5 mL, 2.5 eq), H₂O (500.00 mg, 27.75 mmol, 0.5 mL, 16.42 eq), DCM (1.32 g, 15.54 mmol, 1 mL, 9.20 eq) and TFA (27.72 g, 243.11 mmol, 18 mL, 143.86 eq) stirred at 25° C. for 3 hr under N₂ atmosphere. TLC indicated tert-butyl N-[(1R)-1-[methyl(2-phenylethyl)carbamoyl]-3-oxo-3-(tritylamino)propyl]carbamate was consumed completely and two new spots formed. The reaction mixture was concentrated under reduced pressure to give compound (2R)-2-amino-N-methyl-N-(2-phenylethyl) butanediamide (0.4 g, crude, TFA) as a white solid.

### Step 3

A mixture of (2R)-2-amino-N-methyl-N-(2-phenylethyl)butanediamide (0.4 g, 1.10 mmol, 1 eq, TFA) in DMF (5 mL) was added DIEA (426.86 mg, 3.30 mmol, 575.28 uL, 3 eq) and octanoyl octanoate (446.55 mg, 1.65 mmol, 1.5 eq) and was stirred at 25° C. for 12 hr under N₂ atmosphere. The residue was purified by prep-HPLC (column: Phenomenex luna C18 100*40 mm*5 um; mobile phase: [water(0.1% TFA)-ACN]; B %: 20%-58%, 8 min) to give compound (2R)-N-methyl-2-(octanoylamino)-N-(2-phenylethyl)butanediamide (210 mg, 619.57 umol, 56.28% yield, 99% purity) as white solid.

### Example 18: (R)-N1-methyl-2-octanamido-N1-ethylsuccinamide

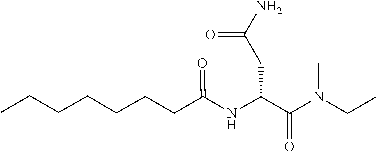 [SMILES: CCCCCCCC(=O)N[C@H](CC(N)=O)C(=O)N(C)CC]

The title compound may be synthesized according to the experimental procedure described for Example 11, replacing tert-butyl amine with ethylmethylamine.

### Example 19: (R)-N1-(tert-butyl)-N1-methyl-2-octanamidosuccinamide

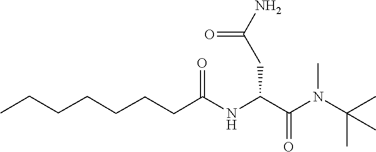 [SMILES: CCCCCCCC(=O)N[C@H](CC(N)=O)C(=O)N(C)C(C)(C)C]

The title compound may be synthesized according to the experimental procedure described for Example 11, replacing tert-butyl amine with tert-butylmethylamine.

### Example 20: (R)-2-(3-hexylureido)-N1-methyl-N1-phenethylsuccinamide

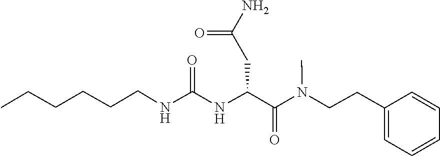 [SMILES: CCCCCCNC(=O)N[C@H](CC(N)=O)C(=O)N(C)CCc1ccccc1]

The title compound may be synthesized according to the experimental procedure described for Example 11, replacing tert-butyl amine with N-methylphenethylamine and octanoic anhydride with hexyl isocyanate.

### Example 21: hexyl (R)-(4-amino-1-(methyl(phenethyl)amino)-1,4-dioxobutan-2-yl)carbamate

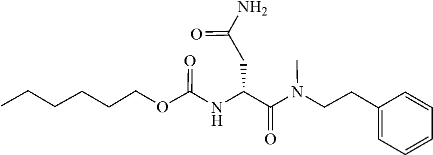 [SMILES: CCCCCCOC(=O)N[C@H](CC(N)=O)C(=O)N(C)CCc1ccccc1]

The title compound may be synthesized according to the experimental procedure described for Example 11, replacing tert-butyl amine with N-methylphenethylamine and octanoic anhydride with hexyl chloroformate.

### Example 22: (S)-1-methoxy-1-oxopropan-2-yl octanoyl-D-asparaginate

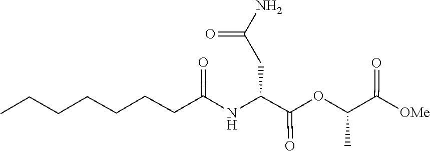 [SMILES: CCCCCCCC(=O)N[C@H](CC(N)=O)C(=O)O[C@@H](C)C(=O)OC]

The title compound was synthesized according to the experimental procedure described for Example 11, replacing tert-butyl amine with methyl (S)-2-hydroxypropanoate and octanoic anhydride with hexyl chloroformate.

¹H NMR (400 MHz, CDCl₃-d) δ 6.71-6.64 (m, 1H), 5.66 (bs, 1H), 5.31 (bs, 1H), 5.19 (q, J=7.1 Hz, 1H), 4.89 (dt, J=8.3, 4.4 Hz, 1H), 3.75 (s, 3H), 2.99 (dd, J=16.3, 4.4 Hz, 1H), 2.83 (dd, J=16.3, 4.5 Hz, 1H), 2.23 (dd, J=8.4, 6.7 Hz, 2H), 1.69-1.53 (m, 2H), 1.50 (d, J=7.0 Hz, 3H), 1.36-1.20 (m, 8H), 0.87 (t, J=6.15 Hz, 3H). LCMS (ESI, m/z): (M+H)⁺ calcd for C₁₆H₂₉N₂O₆, 345.2; found, 345.2.

### Example 23: (R)-N1-ethyl-3-methyl-2-octanamidosuccinamide

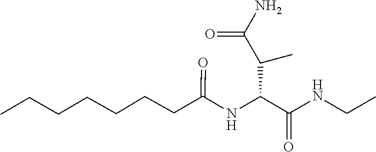 [SMILES: CCCCCCCC(=O)N[C@@H](C(=O)NCC)C(C)C(N)=O]

To be prepared according to Example 11, replacing D-asparagine with β-methyl-D-asparagine and tert-butylamine with ethylamine.

### Example 24: (R)-N1-(tert-butyl)-3-methyl-2-octanamidosuccinamide

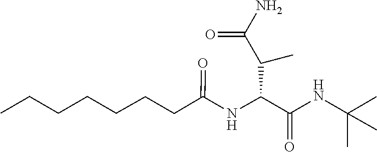 [SMILES: CCCCCCCC(=O)N[C@@H](C(=O)NC(C)(C)C)C(C)C(N)=O]

To be prepared according to Example 11, replacing D-asparagine with β-methyl-D-asparagine.

### Example 25: (R)-3-methyl-2-octanamido-N1-phenethylsuccinamide

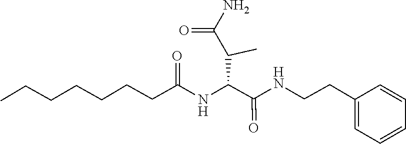 [SMILES: CCCCCCCC(=O)N[C@@H](C(=O)NCCc1ccccc1)C(C)C(N)=O]

To be prepared according to Example 11, replacing D-asparagine with β-methyl-D-asparagine and tert-butylamine with phenethylamine.

### Example 26: (R)-2-(3-hexylureido)-3-methyl-N1-phenethylsuccinamide

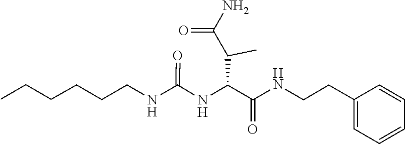 [SMILES: CCCCCCNC(=O)N[C@@H](C(=O)NCCc1ccccc1)C(C)C(N)=O]

To be prepared according to Example 11, replacing D-asparagine with β-methyl-D-asparagine, tert-butylamine with phenethylamine, and octanoic anhydride with hexyl isocyanate.

### Example 27: hexyl ((R)-4-amino-3-methyl-1,4-dioxo-1-(phenethylamino)butan-2-yl)carbamate

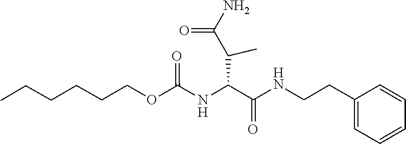 [SMILES: CCCCCCOC(=O)N[C@@H](C(=O)NCCc1ccccc1)C(C)C(N)=O]

To be prepared according to Example 11, replacing D-asparagine with β-methyl-D-asparagine, tert-butylamine with phenethylamine, and octanoic anhydride with hexyl chloroformate.

### Example 28: (R)-N1-ethyl-2-methyl-2-octanamidosuccinamide

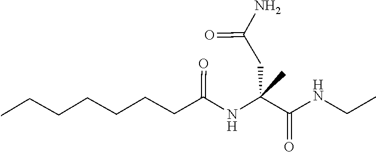 [SMILES: CCCCCCCC(=O)N[C@](C)(CC(N)=O)C(=O)NCC]

To be prepared according to Example 11, replacing D-asparagine with a-methyl-D-asparagine, and tert-butylamine with ethylamine.

### Example 29: (R)-N1-(tert-butyl)-2-methyl-2-octanamidosuccinamide

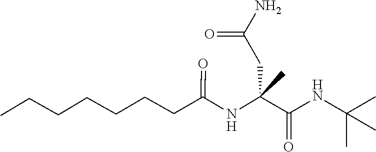 [SMILES: CCCCCCCC(=O)N[C@](C)(CC(N)=O)C(=O)NC(C)(C)C]

To be prepared according to Example 11, replacing D-asparagine with a-methyl-D-asparagine.

### Example 30: (R)-2-methyl-2-octanamido-N1-phenethylsuccinamide

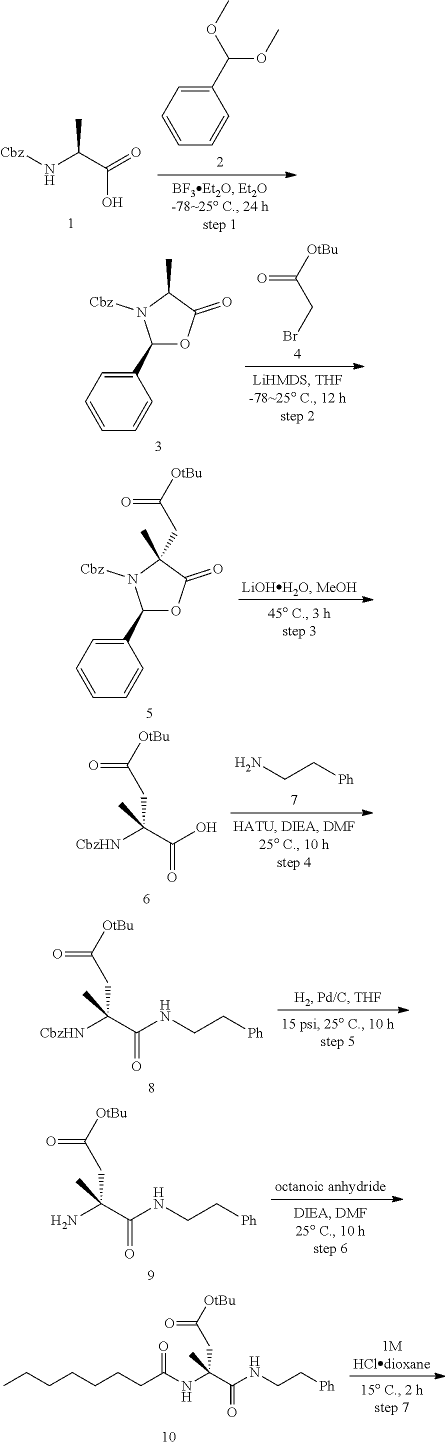

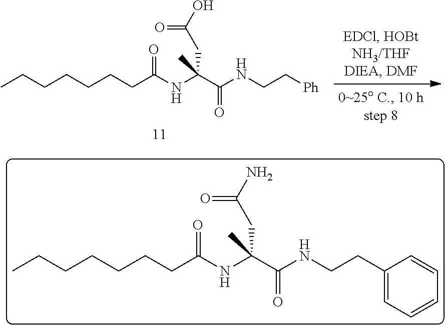 [SMILES: CCCCCCCC(=O)N[C@](C)(CC(=O)O)C(=O)NCCc1ccccc1 ; CCCCCCCC(=O)N[C@](C)(CC(N)=O)C(=O)NCCc1ccccc1]

### Step 1

A solution of (2S)-2-(benzyloxycarbonylamino)propanoic acid (5 g, 22.40 mmol, 1 eq), dimethoxymethylbenzene (3.41 g, 22.40 mmol, 3.38 mL, 1 eq) in Et₂O (35.30 g, 476.26 mmol, 50.00 mL, 21.26 eq) was added BF₃·Et₂O (19.07 g, 134.39 mmol, 16.59 mL, 6 eq) at −78° C. The mixture was warmed to 25° C. and stirred at 25° C. for 24 hr under N₂ atmosphere. The reaction mixture was slowly added to cooled saturated aqueous NaHCO₃ (1000 mL) and stirred for 30 min, then the organic layers was washed H₂O, dried with MgSO₄. The solvent was removed in vacuo to get a residue. The residue was purified by column chromatography (SiO₂, Petroleum ether/Ethyl acetate=20/1 to 10/1) and recrystallization with (Et₂O: PE=1:2). The mixture was filtered and the filter cake was concentrated under reduced pressure to give compound benzyl (2S,4S)-4-methyl-5-oxo-2-phenyl-oxazolidine-3-carboxylate (12 g, 37.77 mmol, 47.04% yield, 98% purity) as colorless crystals.

### Step 2

A solution of LiHMDS (1 M, 12.53 mL, 1.3 eq) in THF (5 mL) was added benzyl (2S,4S)-4-methyl-5-oxo-2-phenyl-oxazolidine-3-carboxylate (3 g, 9.64 mmol, 1 eq) in THF (3 mL) at −78° C. The mixture was stirred at −78° C. for 0.5 hr under N2 atmosphere. Then tert-butyl 2-bromoacetate (2.26 g, 11.56 mmol, 1.71 mL, 1.2 eq) in THF (2 mL) was added at −78° C. The mixture was stirred at 25° C. for 11.5 hr. The reaction mixture was quenched by addition H₂O 500 mL at 0° C., extracted with EtOAc 300 mL (100 mL*3). The combined organic layers were dried over Na₂SO₄, filtered and concentrated under reduced pressure to give a residue. The residue was purified by column chromatography (SiO₂, Petroleum ether/Ethyl acetate=10/1 to 3/1) to give compound benzyl (2S,4R)-4-(2-tert-butoxy-2-oxo-ethyl)-4-methyl-5-oxo-2-phenyl-oxazolidine-3-carboxylate (6 g, 11.42 mmol, 29.64% yield, 81% purity) as white solid.

### Step 3

A solution of benzyl (2S,4R)-4-(2-tert-butoxy-2-oxo-ethyl)-4-methyl-5-oxo-2-phenyl-oxazolidine-3-carboxylate (2 g, 4.70 mmol, 1 eq) in MeOH (4.7 mL) was added LiOH·H₂O (2 M, 4.70 mL, 2 eq). Then the mixture was stirred at 45° C. for 3 hr under N₂ atmosphere. The reaction mixture was diluted with H₂O 60 mL and washed with EtOAc 60 mL(20 mL*3). The aqueous phase was adjusted pH=3 with 1N HCl aqueous. Then the aqueous phase was extracted with EtOAc 60 mL(20 mL*3), dried over Na₂SO₄, filtered and concentrated under reduced pressure to give a residue. The residue was purified by column chromatography (SiO₂, Petroleum ether/Ethyl acetate=10/1 to 5/1) to give compound (2R)-2-(benzyloxycarbonylamino)-4-tert-butoxy-2-methyl-4-oxo-butanoic acid (1 g, 2.96 mmol, 63.06% yield) as colorless oil.

### Step 4

A mixture of (2R)-2-(benzyloxycarbonylamino)-4-tert-butoxy-2-methyl-4-oxo-butanoic acid (0.6 g, 1.78 mmol, 1 eq), 2-phenylethanamine (258.62 mg, 2.13 mmol, 268.00 uL, 1.2 eq), HATU (811.48 mg, 2.13 mmol, 1.2 eq), DIEA (505.67 mg, 3.91 mmol, 681.50 uL, 2.2 eq) in DMF (3 mL) was stirred at 25° C. for 10 hr under N₂ atmosphere. The combined organic phase was diluted with EtOAc 20 mL and washed with water 60 mL (20 mL*3) and brine 40 mL (20 mL*2), dried with anhydrous Na₂SO₄, filtered and concentrated under reduced pressure to give a residue. The residue was purified by column chromatography (SiO₂, Petroleum ether/Ethyl acetate=10/1 to 4/1) to give

compound tert-butyl (3R)-3-(benzyloxycarbonylamino)-3-methyl-4-oxo-4-(2-phenylethylamino) butanoate (0.4 g, 808.12 umol, 45.44% yield, 89% purity) as white solid.

### Step 5

To a solution of methyl tert-butyl (3R)-3-(benzyloxycarbonylamino)-3-methyl-4-oxo-4-(2-phenylethylamino)butanoate (0.4 g, 908.00 umol, 1 eq) in THF (3 mL) was added Pd/C (70 mg, 908.00 umol, 10% purity, 1 eq) under N2. The suspension was degassed under vacuum and purged with H₂ several times. The mixture was stirred under H₂ (15 psi) at 25° C. for 10 hr. The reaction mixture was filtered and the filtrate was concentrated to get a residue. The residue was purified by column chromatography (SiO₂, Petroleum ether/Ethyl acetate=10/1 to 2/1) to give compound tert-butyl (3R)-3-amino-3-methyl-4-oxo-4-(2-phenylethylamino)butanoate (0.24 g, 759.79 umol, 83.68% yield, 97% purity) as colorless oil.

### Step 6

A solution of tert-butyl (3R)-3-amino-3-methyl-4-oxo-4-(2-phenylethylamino)butanoate (0.24 g, 783.29 umol, 1 eq) in DMF (2 mL) was added DIEA (303.70 mg, 2.35 mmol, 409.30 uL, 3 eq), octanoyl octanoate (317.71 mg, 1.17 mmol, 1.5 eq) and was degassed and purged with N₂ for 3 times. The mixture was stirred at 25° C. for 10 hr under N₂ atmosphere. The mixture was diluted with EtOAc 20 mL and washed with water 60 mL (20 mL*3) and brine 40 mL (20 mL*2), dried with anhydrous Na₂SO₄, filtered and concentrated under reduced pressure to give a residue. The residue was purified by column chromatography (SiO₂, Petroleum ether/Ethyl acetate=10/1 to 3/1) to give compound tert-butyl (3R)-3-methyl-3-(octanoylamino)-4-oxo-4-(2-phenylethylamino) butanoate (0.3 g, 658.82 umol, 84.11% yield, 95% purity) as white solid.

### Step 7

A solution of tert-butyl (3R)-3-methyl-3-(octanoylamino)-4-oxo-4-(2-phenylethylamino)butanoate (80 mg, 184.93 umol, 1 eq) in HCl/dioxane (2 M, 2.00 mL, 21.63 eq) was stirred at 15° C. for 2 hr under N₂ atmosphere. The reaction mixture was concentrated under reduced pressure to get a residue. The residue was purified by column chromatography (SiO₂, Petroleum ether/Ethyl acetate=1/1 to Ethyl acetate/MeOH=0/1) to give compound (3R)-3-methyl-3-(octanoylamino)-4-oxo-4-(2-phenylethylamino)butanoic acid (40 mg, crude) as white solid.

### Step 8

A mixture of (3R)-3-methyl-3-(octanoylamino)-4-oxo-4-(2-phenylethylamino)butanoic acid (40 mg, 106.24 umol, 1 eq), HOBt (17.23 mg, 127.49 umol, 1.2 eq) and EDCI (24.44 mg, 127.49 umol, 1.2 eq) in DMF (2 mL) was stirred at 0° C. for 30 min. The mixture was added NH₃·THF (9.4 M, 11.30 uL, 1 eq) and stirred at 25° C. for 9.5 hr under N₂ atmosphere. The reaction mixture was quenched by addition H₂O 20 mL at 0° C. and extracted with EtOAc 30 mL (10 mL*3). The combined organic layers were dried over Na₂SO₄, filtered and concentrated under reduced pressure to give a residue. The residue was purified by prep-HPLC (column: Phenomenex Gemini-NX C18 75*30 mm*3 um; mobile phase: [water(0.05% NH₃H₂O+10 mM NH4HCO3)-ACN]; B %: 20%-60%, 8 min) to give compound (2R)-2-methyl-2-(octanoylamino)-N-(2-phenylethyl)butanediamide (12 mg, 29.08 umol, 27.37% yield, 91% purity) as colorless oil.

### Example 31: (R)-2-(3-hexylureido)-2-methyl-N1-phenethylsuccinamide

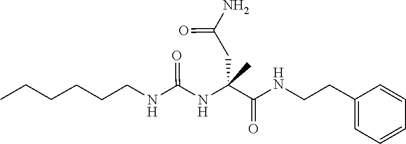 [SMILES: CCCCCCNC(=O)N[C@](C)(CC(N)=O)C(=O)NCCc1ccccc1]

To be prepared according to Example 11, replacing D-asparagine with a-methyl-D-asparagine, tert-butylamine with phenethylamine, and octanoic anhydride with hexyl isocyanate.

### Example 32: hexyl ((R)-4-amino-2-methyl-1,4-dioxo-1-(phenethylamino)butan-2-yl)carbamate

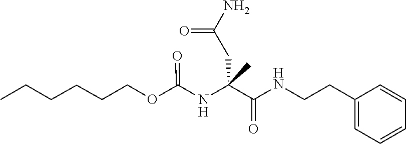 [SMILES: CCCCCCOC(=O)N[C@](C)(CC(N)=O)C(=O)NCCc1ccccc1]

To be prepared according to Example 11, replacing D-asparagine with a-methyl-D-asparagine, tert-butylamine with phenethylamine, and octanoic anhydride with hexyl chloroformate.

### Example 33: methyl ((R)-4-amino-2-octanamido-4-oxobutyl)-L-alaninate

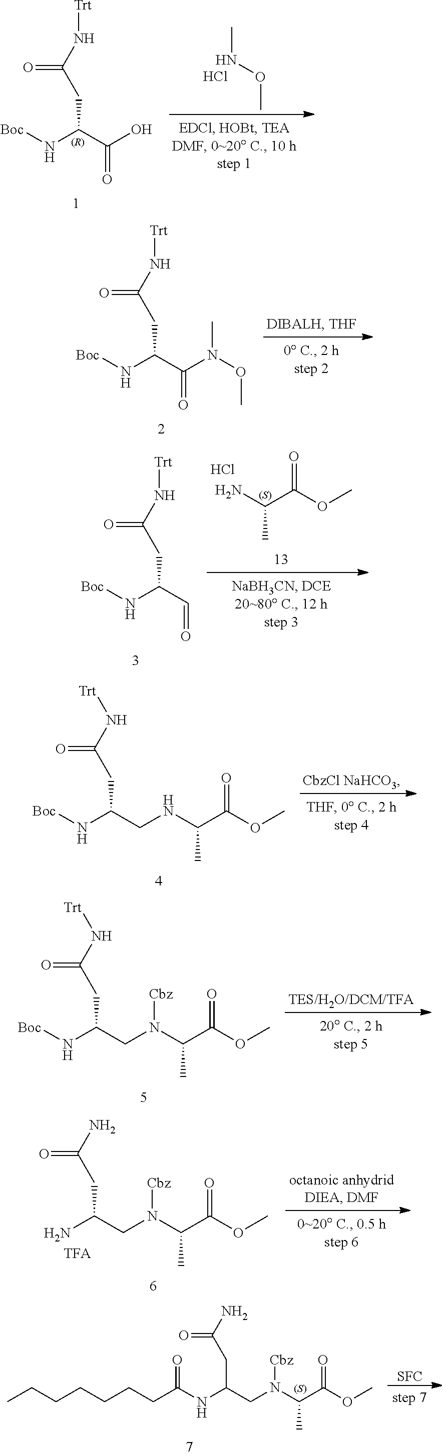

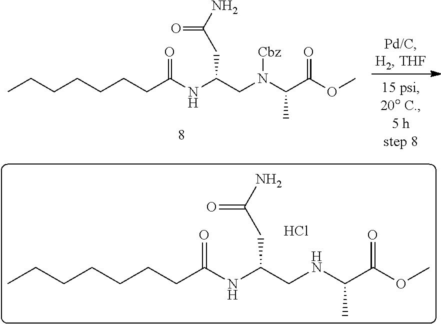

### Step 1

To a solution of (2R)-2-(tert-butoxycarbonylamino)-4-oxo-4-(tritylamino)butanoic acid (4 g, 8.43 mmol, 1 eq) in DMF (50 mL) was added EDCI (1.78 g, 9.27 mmol, 1.1 eq) and HOBt (1.25 g, 9.27 mmol, 1.1 eq) at 0° C. Then N-methoxymethanamine hydrochloride (904.42 mg, 9.27 mmol, 1.1 eq) and TEA (938.22 mg, 9.27 mmol, 1.29 mL, 1.1 eq) was added to the mixture and stirred at 20° C. for 10 hr. TLC indicated one new spot formed. The reaction mixture was diluted by addition H2O 50 mL, and then filtered and the filter cake was concentrated under reduced pressure to give a residue. Compound tert-butyl N-[(1R)-1-[methoxy(methyl)carbamoyl]-3-oxo-3-(tritylamino) propyl]carbamate (4.2 g, 7.95 mmol, 94.34% yield, 98% purity) was obtained as a white solid without further purification.

### Step 2

A solution of tert-butyl N-[(1R)-1-[methoxy(methyl)carbamoyl]-3-oxo-3-(tritylamino)propyl]carbamate (2 g, 3.86 mmol, 1 eq) in THF (20 mL) was added DIBAL-H (1 M, 11.59 mL, 3 eq) at 0° C. The mixture was stirred at 0° C. for 2 h. TLC indicated one new spot formed. The reaction mixture was quenched by addition H2O 200 mL at 0° C., and then extracted with EtOAc 300 mL (100 mL*3). The combined organic layers were washed with 0.1N HCl (200 mL), dried over Na2SO4, filtered and the filtrate was concentrated under reduced pressure to give a residue. Compound tert-butyl N-[(1R)-1-formyl-3-oxo-3-(tritylamino)propyl]carbamate (2.2 g, crude) was obtained as a yellow solid without further purification.

### Step 3

To a solution of tert-butyl N-[(1R)-1-formyl-3-oxo-3-(tritylamino)propyl]carbamate (2.2 g, 4.80 mmol, 1 eq) and methyl (2S)-2-aminopropanoate hydrochloride (669.67 mg, 4.80 mmol, 1 eq) in DCE (50 mL) was stirred at 20° C. for 30 min. Then sodium triacetoxyboranuide (2.03 g, 9.60 mmol, 2 eq) was added to the mixture, stirred at 20° C. for 1.5 h and stirred at 80° C. for 10 h. TLC indicated three new spots formed. The combined organic phase was diluted with EtOAc 200 mL and washed with water 60 mL (200 mL*3) and brine 40 mL (200 mL*2), dried with anhydrous Na₂SO₄, filtered and concentrated under reduced pressure to give a residue. The residue was purified by column chromatography (SiO2, Petroleum ether/Ethyl acetate=2/1 to 1/2). Compound methyl (2S)-2-[[(2R)-2-(tert-butoxycarbonylamino)-4-oxo-4-(tritylamino)butyl]amino]propanoate (833 mg, 931.21 umol, 19.41% yield, 61% purity) was obtained as a yellow solid.

### Step 4

A mixture of methyl (2S)-2-[[(2R)-2-(tert-butoxycarbonylamino)-4-oxo-4-(tritylamino)butyl]amino]propanoate (770 mg, 1.41 mmol, 1 eq) in THF (4 mL) was added NaHCO₃ (8.64 g, 102.85 mmol, 4 mL, 72.88 eq) and CbzCl (288.87 mg, 1.69 mmol, 240.72 uL, 1.2 eq) at 0° C. and was degassed and purged with N2 for 3 times. The mixture was stirred at 0° C. for 2 hr under N2 atmosphere. TLC indicated methyl (2S)-2-[[(2R)-2-(tert-butoxycarbonylamino)-4-oxo-4-(tritylamino)butyl]amino]propanoate was consumed completely and one new spot formed. The reaction mixture was diluted with H2O 20 mL and extracted with EtOAc 60 mL(20 mL*3). The combined organic layers were dried over Na₂SO₄, filtered and concentrated under reduced pressure to give a residue. The residue was purified by column chromatography (SiO2, Petroleum ether/Ethyl acetate=5/1 to 2/1). Compound methyl (2S)-2-[benzyloxycarbonyl-[(2R)-2-(tert-butoxycarbonylamino)-4-oxo-4-(tritylamino)butyl]amino]propanoate (650 mg, 879.67 umol, 62.34% yield, 92% purity) was obtained as a white solid.

### Step 5

A mixture of methyl (2S)-2-[benzyloxycarbonyl-[(2R)-2-(tert-butoxycarbonylamino)-4-oxo-4-(tritylamino)butyl]amino]propanoate (300 mg, 441.31 umol, 1 eq) in a mixture of TES (252.92 mg, 1.10 mmol, 2.5 eq), Water (19.88 mg, 1.10 mmol, 19.88 uL, 2.5 eq), TFA (4.53 g, 39.72 mmol, 2.94 mL, 90 eq) and DCM (187.40 mg, 2.21 mmol, 141.97 uL, 5 eq) was stirred at 20° C. for 2 hr under N2 atmosphere. TLC indicated methyl (2S)-2-[benzyloxycarbonyl-[(2R)-2-(tert-butoxycarbonylamino)-4-oxo-4-(tritylamino)butyl]amino]propanoate was consumed completely and one new spot formed. The reaction mixture was concentrated under reduced pressure to get a residue. Compound methyl (2S)-2-[benzyloxycarbonyl-[(2R)-2,4-diamino-4-oxo-butyl]amino]propanoate (300 mg, crude, TFA) was used for next step as a white solid without more purification.

### Step 6

To a solution of methyl (2S)-2-[benzyloxycarbonyl-[(2R)-2,4-diamino-4-oxo-butyl]amino]propanoate (300 mg, 664.61 umol, 1 eq, TFA) in DMF (1 mL) was added TEA (201.76 mg, 1.99 mmol, 277.52 uL, 3 eq) and octanoyl octanoate (359.43 mg, 1.33 mmol, 2 eq) at 0° C. The mixture was stirred at 20° C. for 0.5 hr. TLC showed desired spot. The combined organic phase was diluted with EtOAc 20 mL and washed with water 60 mL (20 mL*3) and brine 40 mL (20 mL*2), dried with anhydrous Na2SO4, filtered and concentrated under reduced pressure to give a residue. The residue was purified by prep-TLC (SiO2, DCM: MeOH=10:1). Compound methyl (2S)-2-[[4-amino-2-(octanoylamino)-4-oxo-butyl]-benzyloxycarbonyl-amino]propanoate (150 mg, 307.40 umol, 46.25% yield, 95% purity) was obtained as a white solid.

### Step 7

Methyl (2S)-2-[[4-amino-2-(octanoylamino)-4-oxo-butyl]-benzyloxycarbonyl-amino]propanoate (280.00 mg, 604.01 umol, 1 eq) was separated by SFC (column: DAICEL CHIRALPAK AS(250 mm*30 mm, 10 um); mobile phase: [0.1% NH3H2O MEOH]; B %: 30%-20%, 10 min). Compound methyl (2S)-2-[[(2R)-4-amino-2-(octanoylamino)-4-oxo-butyl]-benzyloxycarbonyl-amino]propanoate (140 mg, 274.83 umol, 45.50% yield, 91% purity) was obtained as colorless oil.

### Step 8

To a solution of methyl (2S)-2-[[(2R)-4-amino-2-(octanoylamino)-4-oxo-butyl]-benzyloxycarbonyl-amino]propanoate (130 mg, 280.43 umol, 1 eq) in THF (5 mL) was added Pd/C (20 mg, 10% purity) under N2. The suspension was degassed under vacuum and purged with H2 several times. The mixture was stirred under H2 (15 psi) at 20° C. for 5 hr. The reaction mixture was filtered and the filter was concentrated. The residue was purified by prep-TLC (SiO2, DCM: MeOH=10:1) to give the product. The product was freeze-dried after working with HCl (0.25 M, 1.21 mL, 2 eq) to give compound methyl (2S)-2-[[(2R)-4-amino-2-(octanoylamino)-4-oxo-butyl]amino]propanoate (44 mg, 117.85 umol, 77.65% yield, 98% purity, HCl) as a white solid.

Diastereomer 1 (HCl salt): ¹H NMR (400 MHz, DMSO-d₆) δ 9.51 (s, 1H), 9.17 (s, 1H), 8.04 (d, J=7.9 Hz, 1H), 7.51 (s, 1H), 7.02 (s, 1H), 4.32-4.24 (m, 1H), 4.16 (d, J=6.8 Hz, 1H), 3.74 (s, 3H), 3.15-3.11 (m, 1H), 3.06-2.99 (m, 1H), 2.38 (dd, J=6.6, 3.3 Hz, 2H), 2.08 (t, J=7.5 Hz, 2H), 1.53-1.40 (m, 5H), 1.23 (s, 8H), 0.88-0.80 (m, 3H). LCMS (ESI, m/z): (M+H)⁺ calcd for C₁₆H₃₂N₃O₄, 330.2; found, 330.2.

Diastereomer 2 (HCl salt): ¹H NMR (400 MHz, DMSO-d₆) δ 9.37 (s, 1H), 9.31 (s, 1H), 8.01 (d, J=7.8 Hz, 1H), 7.51 (s, 1H), 7.03 (s, 1H), 4.28-4.23 (m, 1H), 4.21-4.14 (m, 1H), 3.74 (s, 3H), 3.12-3.06 (m, 2H), 2.42-2.36 (m, 2H), 2.07 (t, J=7.5 Hz, 2H), 1.51-1.40 (m, 5H), 1.23 (s, 8H), 0.88-0.80 (m, 3H). LCMS (ESI, m/z): (M+H)⁺ calcd for C₁₆H₃₂N₃O₄, 330.2; found, 330.2.

### Example 34: N-(4-amino-4-oxo-1-(phenethylamino)butan-2-yl)octanamide

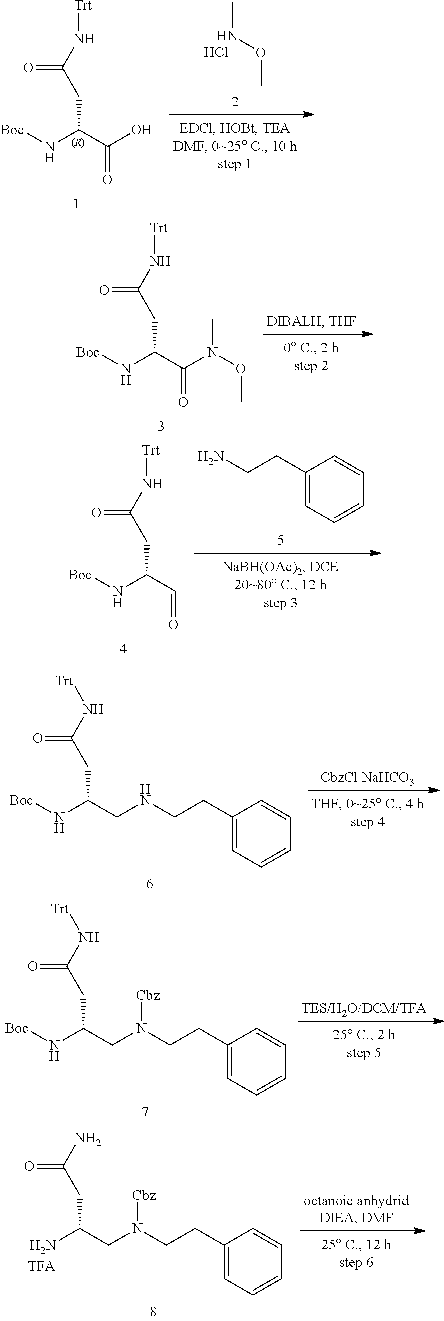

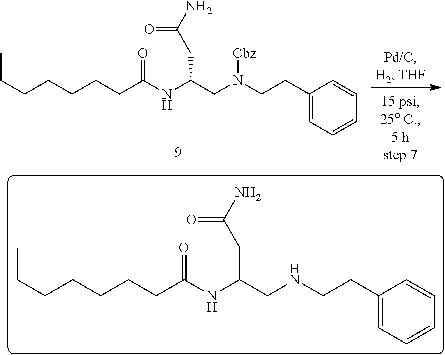 [SMILES: CCCCCCCC(=O)N[C@H](CC(N)=O)CN(CCc1ccccc1)C(=O)OCc1ccccc1 ; CCCCCCCC(=O)NC(CNCCc1ccccc1)CC(N)=O]

### Step 1

To a solution of (2R)-2-(tert-butoxycarbonylamino)-4-oxo-4-(tritylamino)butanoic acid (5 g, 10.54 mmol, 1 eq) in DMF (100 mL) was added EDCI (2.22 g, 11.59 mmol, 1.1 eq) and HOBt (1.57 g, 11.59 mmol, 1.1 eq) at 0° C. The N-methoxymethanamine hydrochloride (1.13 g, 11.59 mmol, 1.1 eq) and TEA (1.17 g, 11.59 mmol, 1.61 mL, 1.1 eq) was added to the mixture at 0° C. and stirred at 25° C. for 10 hr. The reaction mixture was quenched by addition H₂O 200 mL, then filtered and the filter cake was concentrated under reduced pressure to give compound tert-butyl N-[(1R)-1-[methoxy(methyl)carbamoyl]-3-oxo-3-(tritylamino)propyl]carbamate (10 g, 19.32 mmol, 91.68% yield) as white solid.

### Step 2

To a solution of tert-butyl N-[(1R)-1-[methoxy(methyl)carbamoyl]-3-oxo-3-(tritylamino)propyl]carbamate (2 g, 3.86 mmol, 1 eq) in THF (20 mL) was added DIBAL-H (1 M, 11.59 mL, 3 eq) at 0° C. and stirred at 0° C. for 2 h. The reaction mixture was quenched by addition H₂O 300 mL at 0° C., and then extracted with EtOAc 420 mL (140 mL*3). The combined organic layers were washed with 0.1N HCl (400 mL), dried over Na₂SO₄, filtered and the filtrate was concentrated under reduced pressure to give compound tert-butyl N-[1-formyl-3-oxo-3-(tritylamino)propyl]carbamate (1.8 g, crude) as white solid.

### Step 3

A solution of tert-butyl N-[1-formyl-3-oxo-3-(tritylamino)propyl]carbamate (1.8 g, 3.93 mmol, 1 eq) and 2-phenylethanamine (475.68 mg, 3.93 mmol, 492.93 uL, 1 eq) in DCE (10 mL) was stirred at 20° C. for 30 min. The sodium triacetoxyboranuide (1.66 g, 7.85 mmol, 2 eq) was added to the mixture and stirred at 20° C. for 1.5 h. The reaction mixture was warmed to 80° C. and stirred at 80° C. for 10 h. The combined organic phase was diluted with EtOAc 200 mL and washed with water 600 mL (200 mL*3) and brine 400 mL (200 mL*2), dried with anhydrous Na₂SO₄, filtered and concentrated under reduced pressure to give a residue. The residue was purified by column chromatography (SiO₂, Petroleum ether/Ethyl acetate=0/1 to Ethyl acetate: MeOH=10/1) to give compound tert-butyl N-[3-oxo-1-[(2-phenylethylamino)methyl]-3-(tritylamino)propyl]carbamate (1.1 g, 1.33 mmol, 33.80% yield, 68% purity) as yellow solid

### Step 4

To a solution of tert-butyl N-[3-oxo-1-[(2-phenylethylamino)methyl]-3-(tritylamino)propyl]carbamate (1 g, 1.77 mmol, 1 eq) in THF (20 mL) was added NaHCO₃ (39.27 g, 467.48 mmol, 18.18 mL, 263.53 eq) and CbzCl (363.13 mg, 2.13 mmol, 302.61 uL, 1.2 eq) at 0° C. The mixture was stirred at 25° C. for 5 hr under N₂ atmosphere. The reaction mixture was diluted with H₂O 100 mL and extracted with EtOAc 600 mL(200 mL*3). The combined organic layers were dried over Na₂SO₄, filtered and concentrated under reduced pressure to give a residue. The residue was purified by column chromatography (SiO₂, Petroleum ether/Ethyl acetate=5/1 to 2/1) to give compound benzyl N-[2-(tert-butoxycarbonylamino)-4-oxo-4-(tritylamino)butyl]-N-(2-phenyl ethyl)carbamate (0.7 g, 952.91 umol, 53.72% yield, 95% purity) as a white solid.

### Step 5

A mixture of benzyl N-[2-(tert-butoxycarbonylamino)-4-oxo-4-(tritylamino)butyl]-N-(2-phenylethyl)carbamate (600 mg, 859.77 umol, 1 eq), TES (0.25 mL), H₂O (0.25 mL), DCM (0.5 mL) and TFA (9 mL) was stirred at 25° C. for 2 hr under N₂ atmosphere. The reaction mixture was concentrated under reduced pressure to give compound benzyl N-(2,4-diamino-4-oxo-butyl)-N-(2-phenylethyl)carbamate (400 mg, crude, TFA) as white solid.

### Step 6

To a solution of benzyl N-(2,4-diamino-4-oxo-butyl)-N-(2-phenylethyl)carbamate (400 mg, 852.06 umol, 1 eq, TFA) in DMF (3 mL) was added octanoyl octanoate (345.60 mg, 1.28 mmol, 1.5 eq), DIEA (330.36 mg, 2.56 mmol, 445.23 uL, 3 eq). The mixture was stirred at 25° C. for 12 hr under N₂ atmosphere. The combined organic phase was diluted with EtOAc 60 mL and washed with water 180 mL (60 mL*3) and brine 120 mL (60 mL*2), dried with anhydrous Na₂SO₄, filtered and concentrated under reduced pressure to give a residue. Then crude product was washed with the mixture of Petroleum ether (20 mL) and Ethyl acetate (0.2 mL). The mixture was filtered and the filter cake was concentrated under reduced pressure to give compound benzyl N-[4-amino-2-(octanoylamino)-4-oxo-butyl]-N-(2-phenylethyl)carbamate (230 mg, 429.79 umol, 50.44% yield, 90% purity) as white solid.

### Step 7

To a solution of benzyl N-[4-amino-2-(octanoylamino)-4-oxo-butyl]-N-(2-phenylethyl)carbamate (200.00 mg, 415.26 umol, 1 eq) in THF (5 mL) was added Pd/C (20 mg, 64.72 umol, 10% purity) under N₂. The suspension was degassed under vacuum and purged with H₂ several times. The mixture was stirred under H₂ (15 psi) at 25° C. for 5 hr. The reaction mixture was filtered and the filter was concentrated to get a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 30%-55%, 8 min) to give compound N-[3-amino-3-oxo-1-[(2-phenylethylamino)methyl]propyl]octanamide (30 mg, 75.97 umol, 18.30% yield, 88% purity) as white solid.

### Example 35: (R)-N-(4-amino-1-(ethylamino)-4-oxobutan-2-yl)octanamide

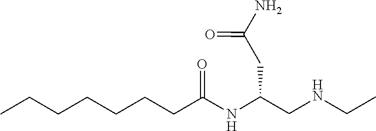 [SMILES: CCCCCCCC(=O)N[C@@H](CNCC)CC(N)=O]

To be prepared according to procedure for Example 33, replacing L-alanine methyl ester with ethylamine.

### Example 36: (R)-N-(4-amino-1-(tert-butylamino)-4-oxobutan-2-yl)octanamide

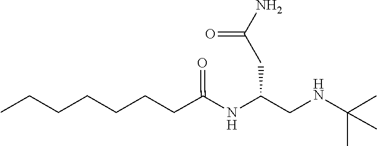 [SMILES: CCCCCCCC(=O)N[C@@H](CNC(C)(C)C)CC(N)=O]

To be prepared according to procedure for Example 33, replacing L-alanine methyl ester with tert-butylamine.

### Example 37: (R)-3-(3-hexylureido)-4-(phenethylamino)butanamide

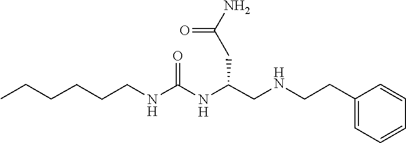 [SMILES: CCCCCCNC(=O)N[C@@H](CNCCc1ccccc1)CC(N)=O]

To be prepared according to procedure for Example 33, replacing L-alanine methyl ester with phenethylamine and octanoic anhydride with hexyl isocyanate.

### Example 38: hexyl (R)-(4-amino-4-oxo-1-(phenethylamino)butan-2-yl)carbamate

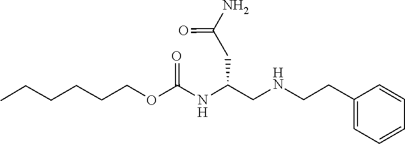 [SMILES: CCCCCCOC(=O)N[C@@H](CNCCc1ccccc1)CC(N)=O]

To be prepared according to procedure for Example 33, replacing L-alanine methyl ester with phenethylamine and octanoic anhydride with hexyl chloroformate.

### Example 39: (R)-N-(4-amino-1,4-dioxobutan-2-yl)octanamide

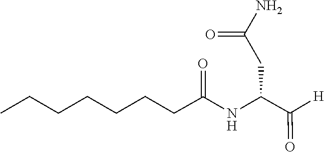 [SMILES: CCCCCCCC(=O)N[C@@H](C=O)CC(N)=O]

Step 1: tert-butyl (R)-(1-(methoxy(methyl)amino)-1,4-dioxo-4-(tritylamino)butan-2-yl)carbamate is treated with TFA, triethylsilane, CH₂Cl₂, and water at room temperature for 2 h to afford (R)-2-amino-N1-methoxy-N1-methylsuccinamide.

Step 2: (R)-2-amino-N1-methoxy-N1-methylsuccinamide is allowed to react with octanoic anhydride, DIPEA, and DMF at 0° C. then warmed to room temperature to afford (R)-N1-methoxy-N1-methyl-2-octanamidosuccinamide.

Step 3: (R)-N1-methoxy-N1-methyl-2-octanamidosuccinamide is treated with DIBAL-H and THF to afford the title compound (R)-N-(4-amino-1,4-dioxobutan-2-yl)octanamide.

### Example 40: (R)-3-(3-hexylureido)-4-oxobutanamide

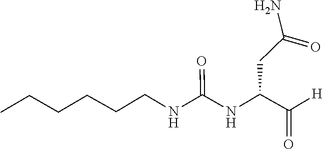 [SMILES: CCCCCCNC(=O)N[C@@H](C=O)CC(N)=O]

The title compound (R)-3-(3-hexylureido)-4-oxobutanamide is prepared following according to the procedures outlined for (R)-N-(4-amino-1,4-dioxobutan-2-yl)octanamide, using 1-isocyanatohexane in place of octanoic anhydride.

### Example 41: hexyl (R)-(4-amino-1,4-dioxobutan-2-yl)carbamate

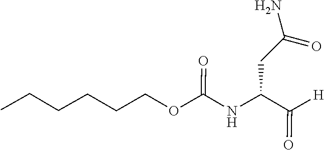 [SMILES: CCCCCCOC(=O)N[C@@H](C=O)CC(N)=O]

The title compound hexyl (R)-(4-amino-1,4-dioxobutan-2-yl)carbamate is prepared following according to the procedures outlined for (R)-N-(4-amino-1,4-dioxobutan-2-yl)octanamide, using hexylchloroformate in place of octanoic anhydride.

### Example 42: (R)-N-(5-amino-1,1,1-trifluoro-2,5-dioxopentan-3-yl)octanamide

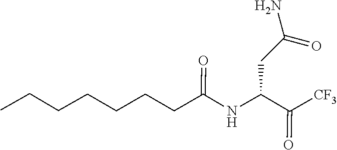 [SMILES: CCCCCCCC(=O)N[C@H](CC(N)=O)C(=O)C(F)(F)F]

Step 1: (R)-(1-(methoxy(methyl)amino)-1,4-dioxo-4-(tritylamino)butan-2-yl)carbamate is allowed to react with LAH and THF to afford tert-butyl (R)-(1,4-dioxo-4-(tritylamino)butan-2-yl)carbamate.

Step 2: tert-butyl (R)-(1,4-dioxo-4-(tritylamino)butan-2-yl)carbamate is treated with trifluoromethyltrimethylsilane, TBAF, and THF to afford tert-butyl ((3R)-1,1,1-trifluoro-2-hydroxy-5-oxo-5-(tritylamino)pentan-3-yl)carbamate.

Step 3: tert-butyl ((3R)-1,1,1-trifluoro-2-hydroxy-5-oxo-5-(tritylamino)pentan-3-yl)carbamate is treated with TFA and CH₂Cl₂ to afford (3R)-3-amino-5,5,5-trifluoro-4-hydroxypentanamide.

Step 4: tert-butyl ((3R)-1,1,1-trifluoro-2-hydroxy-5-oxo-5-(tritylamino)pentan-3-yl)carbamate is allowed to react with octanoic anhydride, DIPEA, and DMF at 0° C. then warmed to room temperature to afford N-((3R)-5-amino-1,1,1-trifluoro-2-hydroxy-5-oxopentan-3-yl)octanamide.

Step 5: N-((3R)-5-amino-1,1,1-trifluoro-2-hydroxy-5-oxopentan-3-yl)octanamide is treated with Dess-Martin periodinane and CH₂Cl₂ to afford the title compound (R)-N-(5-amino-1,1,1-trifluoro-2,5-dioxopentan-3-yl)octanamide.

### Example 43: (R)-5,5,5-trifluoro-3-(3-hexylureido)-4-oxopentanamide

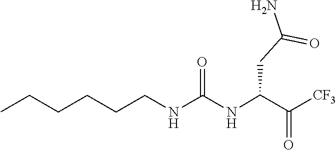 [SMILES: CCCCCCNC(=O)N[C@H](CC(N)=O)C(=O)C(F)(F)F]

The title compound (R)-5,5,5-trifluoro-3-(3-hexylureido)-4-oxopentanamide is prepared following according to the procedures outlined for (R)-N-(5-amino-1,1,1-trifluoro-2,5-dioxopentan-3-yl)octanamide, using 1-isocyanatohexane in place of octanoic anhydride.

### Example 44: hexyl (R)-(5-amino-1,1,1-trifluoro-2,5-dioxopentan-3-yl)carbamate

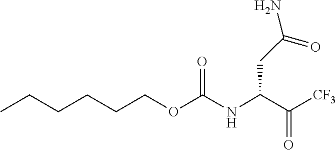 [SMILES: CCCCCCOC(=O)N[C@H](CC(N)=O)C(=O)C(F)(F)F]

The title compound hexyl (R)-(5-amino-1,1,1-trifluoro-2,5-dioxopentan-3-yl)carbamate is prepared following according to the procedures outlined for (R)-N-(5-amino-1,1,1-trifluoro-2,5-dioxopentan-3-yl)octanamide, using hexylchloroformate in place of octanoic anhydride.

### Example 45: (R)-N-(1-amino-5-chloro-1,4-dioxopentan-3-yl)octanamide

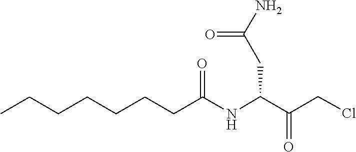 [SMILES: CCCCCCCC(=O)N[C@H](CC(N)=O)C(=O)CCl]

Step 1: N2-(tert-butoxycarbonyl)-N4-trityl-D-asparagine is treated with benzyl alcohol, carbonyl diimidazole, DMF, and CH₂Cl₂ to give benzyl N2-(tert-butoxycarbonyl)-N4-trityl-D-asparaginate.

Step 2: Benzyl N2-(tert-butoxycarbonyl)-N4-trityl-D-asparaginate is treated with TFA, triethylsilane, CH₂Cl₂, and water at room temperature for 2 h to afford benzyl D-asparaginate.

Step 3: Benzyl D-asparaginate is allowed to react with octanoic anhydride, DIPEA, and DMF at 0° C. then warmed to room temperature to afford benzyl octanoyl-D-asparaginate.

Step 4: Octanoyl-D-asparaginate is treated with trimethylsulfoxonium chloride, KOtBu, and DMF to afford (R)-(5-amino-3-octanamido-2,5-dioxopentyl)dimethyl-14-sulfanolate.

Step 5: (R)-(5-amino-3-octanamido-2,5-dioxopentyl)dimethyl-14-sulfanolate is treated with LiCl and methanesulfonic acid to give the title compound (R)-N-(1-amino-5-chloro-1,4-dioxopentan-3-yl)octanamide.

### Example 46: (R)-5-chloro-3-(3-hexylureido)-4-oxopentanamide

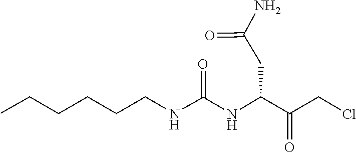 [SMILES: CCCCCCNC(=O)N[C@H](CC(N)=O)C(=O)CCl]

The title compound (R)-5-chloro-3-(3-hexylureido)-4-oxopentanamide is prepared following according to the procedures outlined for (R)-N-(1-amino-5-chloro-1,4-dioxopentan-3-yl)octanamide, using 1-isocyanatohexane in place of octanoic anhydride.

### Example 47: hexyl (R)-(1-amino-5-chloro-1,4-dioxopentan-3-yl)carbamate

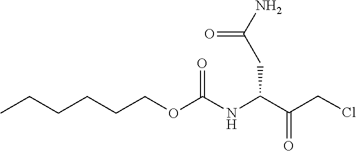 [SMILES: CCCCCCOC(=O)N[C@H](CC(N)=O)C(=O)CCl]

The title compound hexyl (R)-(1-amino-5-chloro-1,4-dioxopentan-3-yl)carbamate is prepared following according to the procedures outlined for (R)-N-(1-amino-5-chloro-1,4-dioxopentan-3-yl)octanamide, using hexylchloroformate in place of octanoic anhydride.

### Example 48: (R,E)-N-(1-amino-1-oxo-7-phenylhept-4-en-3-yl)octanamide

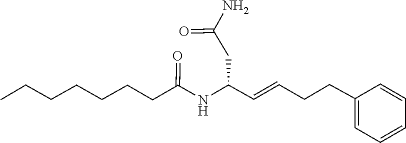 [SMILES: CCCCCCCC(=O)N[C@@H](/C=C/CCc1ccccc1)CC(N)=O]

Step 1: tert-butyl (R)-(1,4-dioxo-4-(tritylamino)butan-2-yl)carbamate is allowed to reacted with (3-phenylpropyl)triphenylphosphonium bromide, NaHMDS, and THF to give tert-butyl (R,E)-(1-oxo-7-phenyl-1-(tritylamino)hept-4-en-3-yl)carbamate.

Step 2: tert-butyl (R,E)-(1-oxo-7-phenyl-1-(tritylamino)hept-4-en-3-yl)carbamate is treated with TFA, triethylsilane, CH₂Cl₂, and water to afford (R,E)-3-amino-7-phenylhept-4-enamide.

Step 3: (R,E)-3-amino-7-phenylhept-4-enamide is allowed to react with octanoic anhydride, DIPEA, and DMF at 0° C. then warmed to room temperature to afford the title compound (R,E)-N-(1-amino-1-oxo-7-phenylhept-4-en-3-yl)octanamide.

### Example 49: (R,E)-3-(3-hexylureido)-7-phenylhept-4-enamide

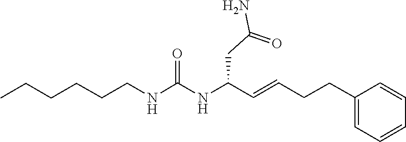 [SMILES: CCCCCCNC(=O)N[C@@H](/C=C/CCc1ccccc1)CC(N)=O]

The title compound (R,E)-3-(3-hexylureido)-7-phenylhept-4-enamide is prepared following according to the procedures outlined for (R,E)-N-(1-amino-1-oxo-7-phenylhept-4-en-3-yl)octanamide, using 1-isocyanatohexane in place of octanoic anhydride.

### Example 50: hexyl (R,E)-(1-amino-1-oxo-7-phenylhept-4-en-3-yl)carbamate

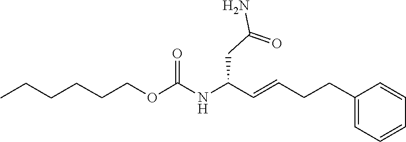 [SMILES: CCCCCCOC(=O)N[C@@H](/C=C/CCc1ccccc1)CC(N)=O]

The title compound hexyl (R,E)-(1-amino-1-oxo-7-phenylhept-4-en-3-yl)carbamate is prepared following according to the procedures outlined for (R,E)-N-(1-amino-1-oxo-7-phenylhept-4-en-3-yl)octanamide, using hexylchloroformate in place of octanoic anhydride.

### Example 51: (R,E)-N-(1-amino-1-oxohept-4-en-3-yl)octanamide

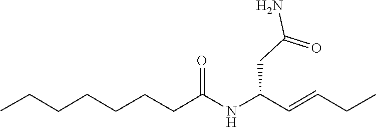 [SMILES: CC/C=C/[C@@H](CC(N)=O)NC(=O)CCCCCCC]

The title compound (R,E)-N-(1-amino-1-oxohept-4-en-3-yl)octanamide is prepared following according to the procedures outlined for (R,E)-N-(1-amino-1-oxo-7-phenylhept-4-en-3-yl)octanamide, using propyltriphenylphosphonium bromide in place of (3-phenylpropyl)triphenylphosphonium bromide.

### Example 52: (R,E)-N-(1-amino-6,6-dimethyl-1-oxohept-4-en-3-yl)octanamide

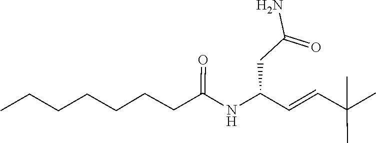 [SMILES: CCCCCCCC(=O)N[C@@H](/C=C/C(C)(C)C)CC(N)=O]

The title compound (R,E)-N-(1-amino-6,6-dimethyl-1-oxohept-4-en-3-yl)octanamide is prepared following according to the procedures outlined for (R,E)-N-(1-amino-1-oxo-7-phenylhept-4-en-3-yl)octanamide, using neopentyl triphenylphosphonium bromide in place of (3-phenylpropyl)triphenylphosphonium bromide.

### Example 53: N-((3R)-1-amino-5-chloro-1,4-dioxo-7-phenylheptan-3-yl)octanamide

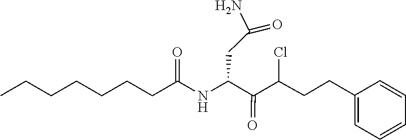 [SMILES: CCCCCCCC(=O)N[C@H](CC(N)=O)C(=O)C(Cl)CCc1ccccc1]

Step 1: (R,E)-N-(1-amino-1-oxo-7-phenylhept-4-en-3-yl)octanamide is treated with m-CPBA and CH₂Cl₂ to afford N-((1R)-3-amino-3-oxo-1-(3-phenethyloxiran-2-yl)propyl)octanamide.

Step 2: N-((1R)-3-amino-3-oxo-1-(3-phenethyloxiran-2-yl)propyl)octanamide is treated with dimethylsulfoxonium chloride, TEA, and CH₂Cl₂ to give the title compound N-((3R)-1-amino-5-chloro-1,4-dioxo-7-phenylheptan-3-yl)octanamide.

### Example 54: (3R)-5-chloro-3-(3-hexylureido)-4-oxo-7-phenylheptanamide

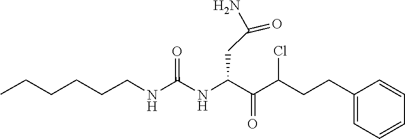 [SMILES: CCCCCCNC(=O)N[C@H](CC(N)=O)C(=O)C(Cl)CCc1ccccc1]

The title compound (3R)-5-chloro-3-(3-hexylureido)-4-oxo-7-phenylheptanamide is prepared following according to the procedures outlined for N-((3R)-1-amino-5-chloro-1,4-dioxo-7-phenylheptan-3-yl)octanamide, using (R)-3-(3-hexylureido)-4-oxobutanamide in place of tert-butyl (R)-(1,4-dioxo-4-(tritylamino)butan-2-yl)carbamate.

### Example 55: hexyl ((3R)-1-amino-5-chloro-1,4-dioxo-7-phenylheptan-3-yl)carbamate

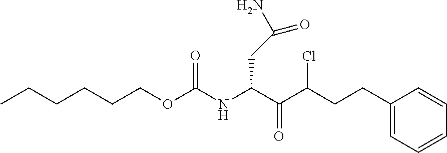 [SMILES: CCCCCCOC(=O)N[C@H](CC(N)=O)C(=O)C(Cl)CCc1ccccc1]

The title compound hexyl ((3R)-1-amino-5-chloro-1,4-dioxo-7-phenylheptan-3-yl)carbamate is prepared following according to the procedures outlined for N-((3R)-1-amino-5-chloro-1,4-dioxo-7-phenylheptan-3-yl)octanamide, using (R)-3-(3-hexylureido)-4-oxobutanamide in place of tert-butyl (R)-(1,4-dioxo-4-(tritylamino)butan-2-yl)carbamate.

### Example 56: (R,E)-N-(1-amino-1,6-dioxo-7-phenylhept-4-en-3-yl)octanamide

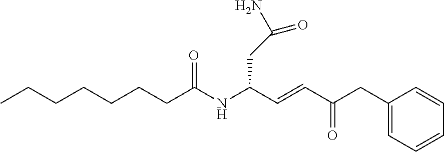 [SMILES: CCCCCCCC(=O)N[C@@H](/C=C/C(=O)Cc1ccccc1)CC(N)=O]

(R)-N-(4-amino-1,4-dioxobutan-2-yl)octanamide is treated with dimethyl (2-oxo-3-phenylpropyl)phosphonate, NaHMDS, and THF to afford the title compound (R,E)-N-(1-amino-1,6-dioxo-7-phenylhept-4-en-3-yl)octanamide.

### Example 57: (R,E)-3-(3-hexylureido)-6-oxo-7-phenylhept-4-enamide

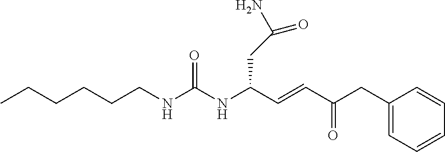 [SMILES: CCCCCCNC(=O)N[C@@H](/C=C/C(=O)Cc1ccccc1)CC(N)=O]

(R)-3-(3-hexylureido)-4-oxobutanamide is treated with dimethyl (2-oxo-3-phenylpropyl)phosphonate, NaHMDS, and THF to afford the title compound (R,E)-3-(3-hexylureido)-6-oxo-7-phenylhept-4-enamide.

### Example 58: hexyl (R,E)-(1-amino-1,6-dioxo-7-phenylhept-4-en-3-yl)carbamate

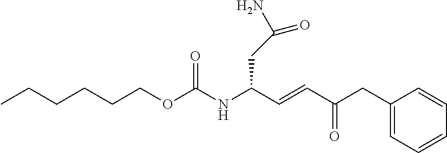 [SMILES: CCCCCCOC(=O)N[C@@H](/C=C/C(=O)Cc1ccccc1)CC(N)=O]

Hexyl (R)-(4-amino-1,4-dioxobutan-2-yl)carbamate is treated with dimethyl (2-oxo-3-phenylpropyl)phosphonate, NaHMDS, and THF to afford the title compound hexyl (R,E)-(1-amino-1,6-dioxo-7-phenylhept-4-en-3-yl)carbamate.

### Example 59: (R,E)-N-(1-amino-1,6-dioxohept-4-en-3-yl)octanamide

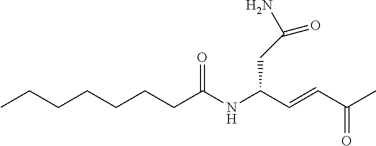 [SMILES: CCCCCCCC(=O)N[C@@H](/C=C/C(C)=O)CC(N)=O]

(R)-N-(4-amino-1,4-dioxobutan-2-yl)octanamide is treated with 1-dimethoxyphosphorylpropan-2-one, NaHMDS, and THF to afford the title compound (R,E)-N-(1-amino-1,6-dioxohept-4-en-3-yl)octanamide.

### Example 60: (R,E)-7,7-dimethyl-3-octanamido-6-oxooct-4-enamide

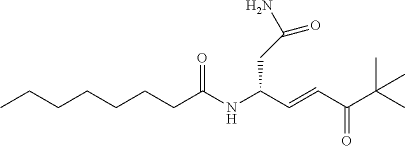 [SMILES: CCCCCCCC(=O)N[C@@H](/C=C/C(=O)C(C)(C)C)CC(N)=O]

(R)-N-(4-amino-1,4-dioxobutan-2-yl)octanamide is treated with diethyl (3,3-dimethyl-2-oxobutyl)phosphonate, NaHMDS, and THF to afford the title compound (R,E)-N-(1-amino-1,6-dioxohept-4-en-3-yl)octanamide.

### Example 61: (R,Z)-N-(1-amino-4-fluoro-1-oxo-7-phenylhept-4-en-3-yl)octanamide

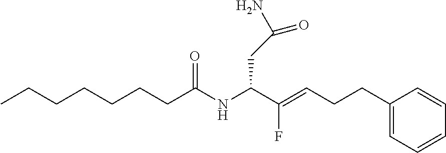 [SMILES: CCCCCCCC(=O)N[C@H](CC(N)=O)/C(F)=C/CCc1ccccc1]

(R)-N-(1-amino-1,4-dioxo-7-phenylheptan-3-yl)octanamide is treated with DAST and CH₂Cl₂ to afford the title compound (R,Z)-N-(1-amino-4-fluoro-1-oxo-7-phenylhept-4-en-3-yl)octanamide.

### Example 62: (R,Z)-4-fluoro-3-(3-hexylureido)-7-phenylhept-4-enamide

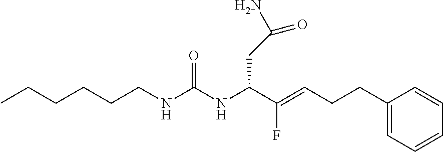 [SMILES: CCCCCCNC(=O)N[C@H](CC(N)=O)/C(F)=C/CCc1ccccc1]

(R)-3-(3-Hexylureido)-4-oxo-7-phenylheptanamide is treated with DAST and CH₂Cl₂ to afford the title compound (R,Z)-4-fluoro-3-(3-hexylureido)-7-phenylhept-4-enamide.

### Example 63: hexyl (R,Z)-(1-amino-4-fluoro-1-oxo-7-phenylhept-4-en-3-yl)carbamate

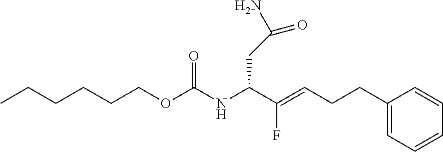 [SMILES: CCCCCCOC(=O)N[C@H](CC(N)=O)/C(F)=C/CCc1ccccc1]

Hexyl (R)-(1-amino-1,4-dioxo-7-phenylheptan-3-yl)carbamate is treated with DAST and CH₂Cl₂ to afford the title compound hexyl (R,Z)-(1-amino-4-fluoro-1-oxo-7-phenylhept-4-en-3-yl)carbamate.

### Example 64: (R,Z)-N-(1-amino-4-fluoro-1-oxohept-4-en-3-yl)octanamide

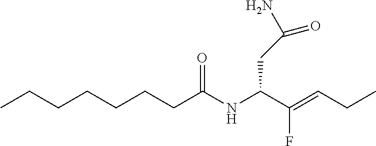 [SMILES: CC/C=C(\F)[C@@H](CC(N)=O)NC(=O)CCCCCCC]

(R)-N-(1-amino-1,4-dioxoheptan-3-yl)octanamide is treated with DAST and CH₂Cl₂ to afford the title compound (R,Z)-N-(1-amino-4-fluoro-1-oxohept-4-en-3-yl)octanamide.

### Example 65: (R,Z)-N-(1-amino-4-fluoro-6,6-dimethyl-1-oxohept-4-en-3-yl)octanamide

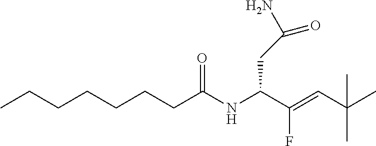 [SMILES: CCCCCCCC(=O)N[C@H](CC(N)=O)/C(F)=C/C(C)(C)C]

(R)-N-(1-amino-6,6-dimethyl-1,4-dioxoheptan-3-yl)octanamide is treated with DAST and CH₂Cl₂ to afford the title compound (R,Z)-N-(1-amino-4-fluoro-6,6-dimethyl-1-oxohept-4-en-3-yl)octanamide.

### Example 66: Methyl ((R)-5-amino-3-octanamido-2,5-dioxopentanoyl)-L-alaninate

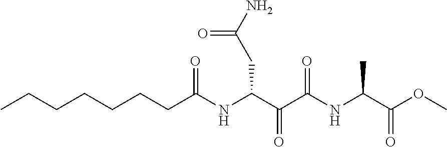 [SMILES: CCCCCCCC(=O)N[C@H](CC(N)=O)C(=O)C(=O)N[C@@H](C)C(=O)OC]

Nα-Boc-Nγ-trityl-D-asparagine (1 equiv) is stirred with (triphenylphosphoranylidene)acetonitrile (1.05 equiv), N-(3-dimethylaminopropyl)-N′-ethylcarbodiimide hydrochloride (1.05 equiv), and 4-(dimethylamino)pyridine (1.05 equiv) in DCM at room temperature overnight. The product (1 equiv) is treated with ozone in DCM at −78° C. for 30 minutes before addition of L-alanine methyl ester hydrochloride (1.05 equiv), and triethylamine (1.05 equiv) in DCM. Deprotection with trifluoroacetic acid (10 equiv) in DCM, followed by reaction with octanoic anhydride (1.05 equiv) and N,N-diisopropylethylamine (1.05 equiv) in DMF for 30 minutes, and purification by reverse-phase C18 column chromatography affords the title compound.

### Example 67: (R)-N1-(tert-butyl)-3-octanamido-2-oxopentanediamide

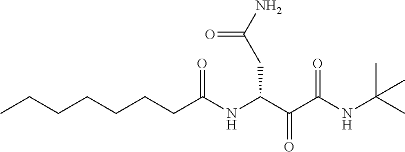 [SMILES: CCCCCCCC(=O)N[C@H](CC(N)=O)C(=O)C(=O)NC(C)(C)C]

The title compound may be synthesized according to the experimental procedure described for Example 66, replacing L-alanine methyl ester hydrochloride with tert-butylamine.

### Example 68: (R)-N1-ethyl-3-octanamido-2-oxopentanediamide

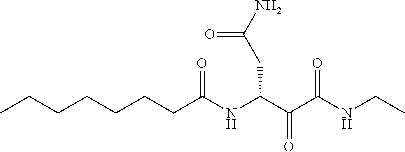 [SMILES: CCCCCCCC(=O)N[C@H](CC(N)=O)C(=O)C(=O)NCC]

The title compound may be synthesized according to the experimental procedure described for Example 66, replacing L-alanine methyl ester hydrochloride with ethylamine.

### Example 69: (R)-3-(3-hexylureido)-2-oxo-N1-phenethylpentanediamide

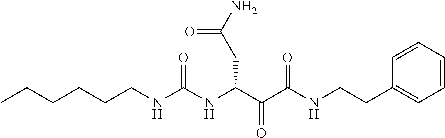 [SMILES: CCCCCCNC(=O)N[C@H](CC(N)=O)C(=O)C(=O)NCCc1ccccc1]

The title compound may be synthesized according to the experimental procedure described for Example 66, replacing octanoic anhydride with hexyl isocyanate and L-alanine methyl ester hydrochloride with ethylamine.

### Example 70: (R)-3-octanamido-2-oxo-N1-phenethylpentanediamide

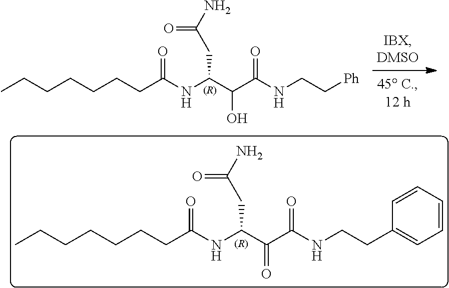

To a solution of (3R)-2-hydroxy-3-(octanoylamino)-N-(2-phenylethyl)pentanediamide (15 mg, 38.31 umol, 1 eq) in DMSO (1 mL) was added IBX (53.64 mg, 191.57 umol, 5 eq). Then the mixture was stirred at 45° C. for 12 hr. The reaction mixture was concentrated under reduced pressure to get a residue. The residue was purified by prep-HPLC (column: Phenomenex Gemini-NX C18 75*30 mm*3 um; mobile phase: [water(10 mM NH₄HCO₃)-ACN]; B %: 20%-50%, 12 min) to give compound (3R)-3-(octanoylamino)-2-oxo-N-(2-phenylethyl) pentanediamide (3 mg, 7.70 umol, 20.10% yield) as a white solid.

### Example 71: Hexyl (R)-(5-amino-1,2,5-trioxo-1-(phenethylamino)pentan-3-yl)carbamate

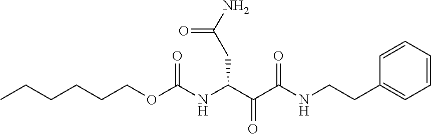 [SMILES: CCCCCCOC(=O)N[C@H](CC(N)=O)C(=O)C(=O)NCCc1ccccc1]

The title compound may be synthesized according to the experimental procedure described for Example 66, replacing octanoic anhydride with hexyl chloroformate and L-alanine methyl ester hydrochloride with phenethylamine.

### Example 72: Methyl ((2-amino-2-oxoethyl)(2-oxononyl)carbamoyl)-L-alaninate

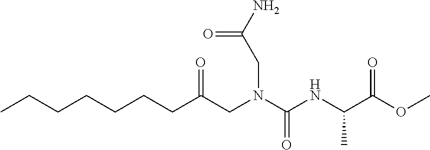 [SMILES: CCCCCCCC(=O)CN(CC(N)=O)C(=O)N[C@@H](C)C(=O)OC]

Step 1: Octanoyl chloride (1 equiv) is reacted with ethyl chloroformate (1.05 equiv), N-methylmorpholine (1.05 equiv), and diazomethane (1.05 equiv) at 0° C. for 3 hours, followed by slow addition of 1M HCl solution in dioxane to yield 1-chlorononan-2-one.

Step 2: Boc-glycinamide (1 equiv) is reacted with trityl chloride (1.05 equiv) in DCM overnight at room temperature. The solution is than stirred with 1-chlorononan-2-one (1.05 equiv) at 80° C. overnight. The product is deprotected with trifluoroacetic acid (5 equiv) in DCM, followed by addition of (2S)-2-isocyanatopropanoic acid methyl ester (1.05 equiv) in DMF. The product is purified by reverse-phase C18 column chromatography to yield the title compound.

### Example 73: 2-(3-ethyl-1-(2-oxononyl)ureido)acetamide

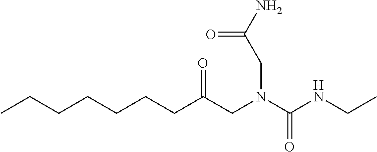 [SMILES: CCCCCCCC(=O)CN(CC(N)=O)C(=O)NCC]

The title compound may be synthesized according to the experimental procedure described for Example 72, replacing (2S)-2-isocyanatopropanoic acid methyl with ethyl isocyanate.

### Example 74: 2-(1-(2-oxononyl)-3-phenethylureido)acetamide

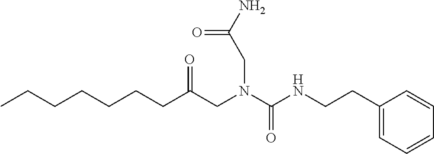 [SMILES: CCCCCCCC(=O)CN(CC(N)=O)C(=O)NCCc1ccccc1]

The title compound may be synthesized according to the experimental procedure described for Example 72, replacing (2S)-2-isocyanatopropanoic acid methyl with phenethyl isocyanate.

### Example 75: 2-(3-neopentyl-1-(2-oxononyl)ureido)acetamide

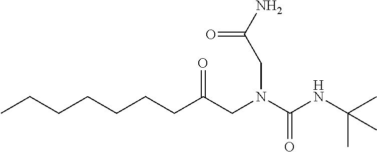 [SMILES: CCCCCCCC(=O)CN(CC(N)=O)C(=O)NC(C)(C)C]

The title compound may be synthesized according to the experimental procedure described for Example 72, replacing (2S)-2-isocyanatopropanoic acid methyl with tert-butyl isocyanate.

### Example 76: Diphenyl (S)-(3-amino-1-octanamido-3-oxopropyl)phosphonate

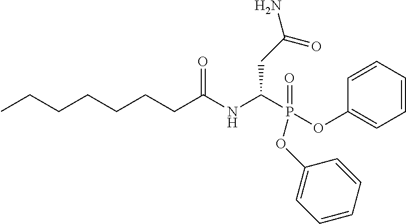 [SMILES: CCCCCCCC(=O)N[C@H](CC(N)=O)P(=O)(Oc1ccccc1)Oc1ccccc1]

N-(bromomethyl)phthalimide (1 equiv) is reacted with diphenylethyl phosphite (1.05 equiv) in xylene at reflux over 5 days, followed by reaction with hydrazine hydrate (1.05 equiv) in acetic acid and tetrahydrofuran at reflux. The resulting product is protected with benzophenone imine (1.05 equiv) in DCM at room temperature overnight. The resulting phosphodiester is reacted with potassium bis(trimethylsilyl)amide (1.05 equiv) and bromoacetamide (1.05 equiv). The benzophenone imine is deprotected with 1M HCl (2 equiv) in dioxane, and the resulting amine is reacted with octanoic anhydride (1.05 equiv) and N,N-diisopropylethylamine (1.05 equiv) in DMF for 30 minutes. Purification by reverse-phase C18 column chromatography affords the title compound.

### Example 77: Hexyl (S)-(3-amino-1-(diphenoxyphosphoryl)-3-oxopropyl)carbamate

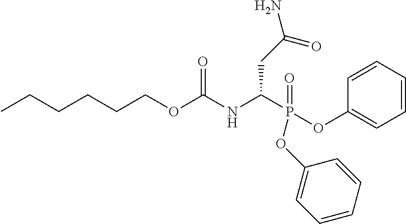 [SMILES: CCCCCCOC(=O)N[C@H](CC(N)=O)P(=O)(Oc1ccccc1)Oc1ccccc1]

The title compound may be synthesized according to the experimental procedure described for Example 76, replacing octanoic anhydride with hexyl chloroformate.

### Example 78: Diphenyl (S)-(3-amino-1-(3-hexylureido)-3-oxopropyl)phosphonate

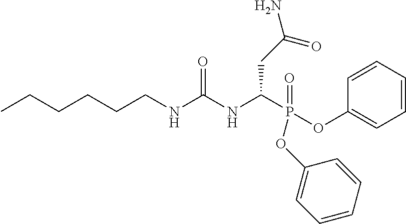 [SMILES: CCCCCCNC(=O)N[C@H](CC(N)=O)P(=O)(Oc1ccccc1)Oc1ccccc1]

The title compound may be synthesized according to the experimental procedure described for Example 76, replacing octanoic anhydride with hexyl isocyanate.

### Example 79: Methyl ((S)-3-amino-1-octanamido-3-oxopropyl)phosphonofluoridate

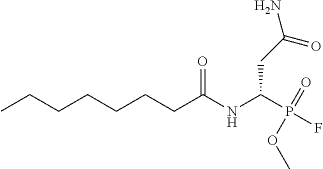 [SMILES: CCCCCCCC(=O)N[C@H](CC(N)=O)P(=O)(F)OC]

The compound in Example 76 (diphenyl (S)-(3-amino-1-octanamido-3-oxopropyl)phosphonate) is reacted with ammonium fluoride (1.05 equiv) in acetonitrile at 60° C., followed by addition of diazomethane (1.05 equiv) in DMSO. The product is purified by reverse phase C18 column chromatography to yield the title compound.

### Example 80: Hexyl ((1S)-3-amino-1-(fluoro(methoxy)phosphoryl)-3-oxopropyl)carbamate

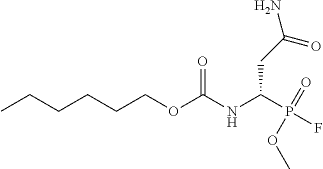 [SMILES: CCCCCCOC(=O)N[C@H](CC(N)=O)P(=O)(F)OC]

The compound in Example 77 (hexyl (S)-(3-amino-1-(diphenoxyphosphoryl)-3-oxopropyl)carbamate) is reacted with ammonium fluoride (1.05 equiv) in acetonitrile at 60° C., followed by addition of diazomethane (1.05 equiv) in DMSO. The product is purified by reverse phase C18 column chromatography to yield the title compound.

### Example 81: Methyl ((S)-3-amino-1-(3-hexylureido)-3-oxopropyl)phosphonofluoridate

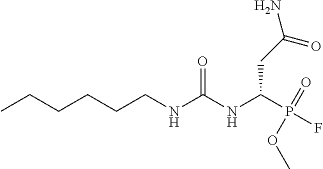 [SMILES: CCCCCCNC(=O)N[C@H](CC(N)=O)P(=O)(F)OC]

The compound in Example 78 (diphenyl (S)-(3-amino-1-(3-hexylureido)-3-oxopropyl)phosphonate) is reacted with ammonium fluoride (1.05 equiv) in acetonitrile at 60° C., followed by addition of diazomethane (1.05 equiv) in DMSO. The product is purified by reverse phase C18 column chromatography to yield the title compound.

### Example 87: Phenethyl (S)-(4-amino-1-(heptylamino)-1,4-dioxobutan-2-yl)carbamate

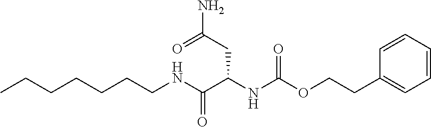 [SMILES: CCCCCCCNC(=O)[C@H](CC(N)=O)NC(=O)OCCc1ccccc1]

Nα-Boc-Nγ-trityl-L-asparagine (1 equiv) is stirred with 1-aminoheptane (1.05 equiv), HATU (1.05 equiv), and N,N-diisopropylethylamine (1.05 equiv) in DMF overnight at room temperature, followed by reaction with TFA (10 equiv) in DCM. The resulting compound is reacted with 2-phenylethyl chloroformate in DMF and purified by C18 column chromatography to yield the title compound.

### Example 88: Heptyl (phenethoxycarbonyl)-L-asparaginate

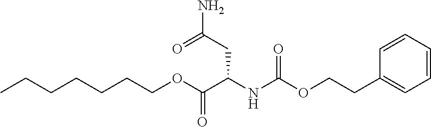 [SMILES: CCCCCCCOC(=O)[C@H](CC(N)=O)NC(=O)OCCc1ccccc1]

The title compound may be synthesized according to the experimental procedure described for Example 87, replacing octanoic anhydride with hexyl chloroformate.

### Example 89: Ethyl (S)-(4-amino-1-(heptylamino)-1,4-dioxobutan-2-yl)carbamate

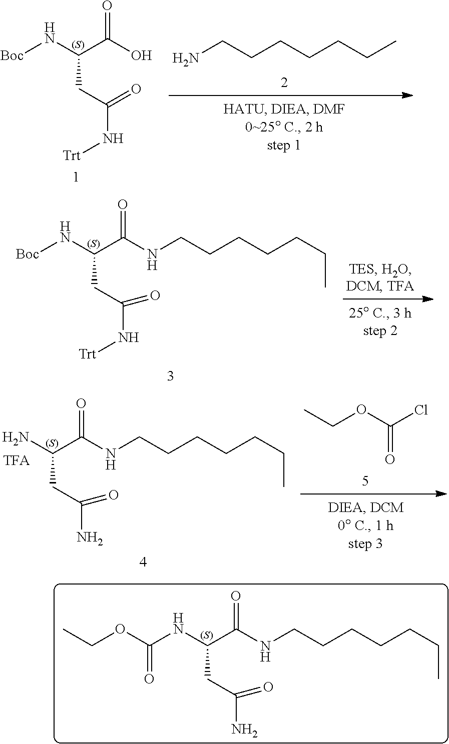

### Step 1

To a solution of (2S)-2-(tert-butoxycarbonylamino)-4-oxo-4-(tritylamino)butanoic acid (300 mg, 632.18 umol, 1 eq) and heptan-1-amine (87.40 mg, 758.62 umol, 113.07 uL, 1.2 eq) in DMF (30 mL) was added HATU (288.45 mg, 758.62 umol, 1.2 eq) and DIPEA (179.75 mg, 1.39 mmol, 242.25 uL, 2.2 eq) at 0° C. The mixture was stirred at 25° C. for 2 hr. TLC showed the starting reactant was consumed and two new spots were formed. The mixture was quenched by addition H₂O 10 mL at 0° C., and then extracted with EtOAc 15 mL (5 mL*3). The combined organic layers were washed with saturated brine 15 mL (5 mL*3), dried over Na₂SO₄, filtered and concentrated under reduced pressure to give a residue. The residue was purified by column chromatography (SiO₂, Petroleum ether/Ethyl acetate=30/1 to 1/1). Compound tert-butyl N-[(1S)-1-(heptylcarbamoyl)-3-oxo-3-(tritylamino)propyl]carbamate (300 mg, 524.71 umol, 83.00% yield) was obtained as a white solid.

### Step 2

The mixture of tert-butyl N-[(1S)-1-(heptylcarbamoyl)-3-oxo-3-(tritylamino)propyl]carbamate (300 mg, 524.71 umol, 1 eq), TES (0.1 mL), H₂O (0.1 mL), DCM (0.2 mL) and TFA (3.6 mL) was stirred at 25° C. for 3 hr. TLC showed the starting reactant was consumed and one new spot was formed. The mixture was concentrated under reduced pressure to give compound (2S)-2-amino-N-heptyl-butanediamide (150 mg, crude, TFA) as a white solid.

### Step 3

To a solution of (2S)-2-amino-N-heptyl-butanediamide (150 mg, 436.88 umol, 1 eq, TFA) in DCM (5 mL) was added DIEA (112.93 mg, 873.77 umol, 152.19 uL, 2 eq) at 0° C. Then it was added ethyl carbonochloridate (52.15 mg, 480.57 umol, 45.75 uL, 1.1 eq) slowly at 0° C. The mixture was stirred at 0° C. for 1 hr. LCMS showed the starting reactant was consumed and have the desired mass. The reaction mixture was quenched by addition H₂O (5 mL) at 0° C., and then extracted with EtOAc 15 mL (5 mL*3). The combined organic layers were washed with saturated brine 15 mL (5 mL*3), dried over Na₂SO₄, filtered and concentrated under reduced pressure to give a residue. The residue was purified by prep-HPLC (column: Phenomenex Luna C18 100*30 mm*5 um; mobile phase: [water(0.1% TFA)-ACN]; B %: 30%-45%, 10 min). Compound ethyl N-[(1S)-3-amino-1-(heptylcarbamoyl)-3-oxo-propyl]carbamate (24 mg, 78.84 umol, 18.05% yield, 99% purity) was obtained as a white solid.

### Example 90: Tert-butyl (S)-(4-amino-1-(heptylamino)-1,4-dioxobutan-2-yl)carbamate

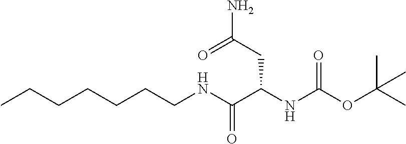 [SMILES: CCCCCCCNC(=O)[C@H](CC(N)=O)NC(=O)OC(C)(C)C]

The title compound may be synthesized according to the experimental procedure described for Example 87, replacing 2-phenylethylchloroformate with tert-butyl chloroformate.

### Example 91: (R,E)-N-(5-amino-1-(methylsulfonyl)-5-oxopent-1-en-3-yl)octanamide

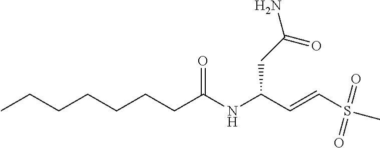 [SMILES: CCCCCCCC(=O)N[C@@H](/C=C/S(C)(=O)=O)CC(N)=O]

Nα-Boc-Nγ-trityl-D-asparagine (1 equiv) is treated with HATU (1.05 equiv), HOAt (1.05 equiv), DIPEA (2.1 equiv), and N,O-dimethylhydroxylamine hydrochloride (1.1 equiv) in DMF overnight at room temperature, followed by reduction with diisobutylaluminium hydride (1.05 equiv) in THF. The aldehyde is treated with a solution of sodium hydride (1.1 equiv) and diethyl (methylsulfonyl)methylphosphonate (1.1 equiv) in THF. The resulting product is deprotected with TFA (10 equiv) in DCM, followed by reaction with octanoic anhydride (1.05 equiv) and N,N-diisopropylethylamine (1.05 equiv) in DMF for 30 minutes. Purification by reverse-phase C18 column chromatography affords the title compound.

### Example 92: Hexyl (R,E)-(5-amino-1-(methylsulfonyl)-5-oxopent-1-en-3-yl)carbamate

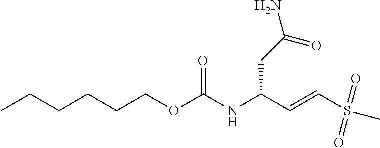 [SMILES: CCCCCCOC(=O)N[C@@H](/C=C/S(C)(=O)=O)CC(N)=O]

The title compound may be synthesized according to the experimental procedure described for Example 91, replacing octanoic anhydride with hexyl chloroformate.

### Example 93: (R,E)-3-(3-hexylureidol-5-(methylsulfonyl)pent-4-enamide

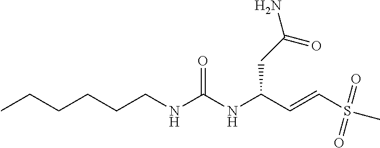 [SMILES: CCCCCCNC(=O)N[C@@H](/C=C/S(C)(=O)=O)CC(N)=O]

The title compound may be synthesized according to the experimental procedure described for Example 91, replacing octanoic anhydride with hexyl isocyanate.

### Example 94: N-((3R,4S)-1-amino-4-hydroxy-1-oxo-7-phenylheptan-3-yl)octanamide and N-((3S,4R)-1-amino-4-hydroxy-1-oxo-7-phenylheptan-3-yl)octanamide

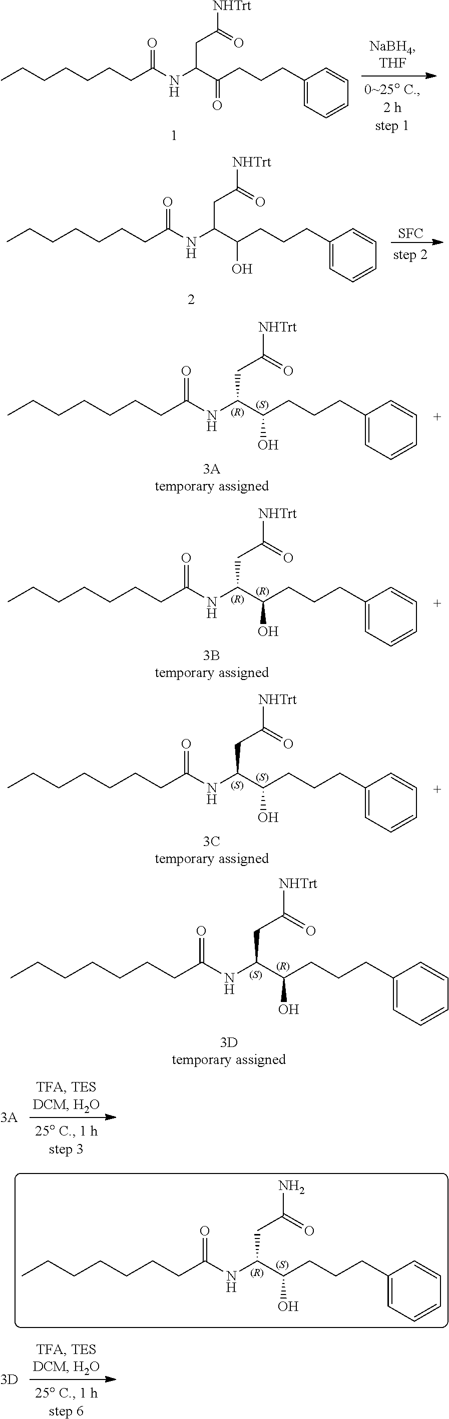

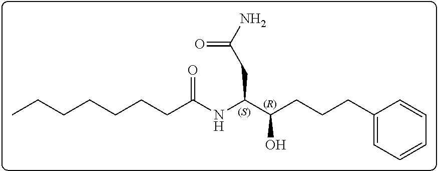

### Step 1

To a solution of N-[(1R)-2-oxo-1-[2-oxo-2-(tritylamino)ethyl]-5-phenyl-pentyl]octanamide (1.30 g, 2.16 mmol, 1 eq) in THF (10 mL) was added NaBH₄ (163.18 mg, 4.31 mmol, 2 eq) at 0° C. The mixture was stirred at 25° C. for 2 hr. TLC showed the starting reactant was consumed and one new spot was formed. The reaction mixture was quenched by addition H₂O 10 mL at 0° C. Then the mixture was extracted with EtOAc 30 mL (10 mL*3). The combined organic layers were washed with saturated brine 30 mL (10 mL*3), dried over Na₂SO₄, filtered and concentrated under reduced pressure to give a residue. The residue was purified by column chromatography (SiO₂, Petroleum ether/Ethyl acetate=30/1 to 3/1) to give compound N-[2-hydroxy-1-[2-oxo-2-(tritylamino)ethyl]-5-phenyl-pentyl]octanamide (900 mg, 1.49 mmol, 69.00% yield) as a white solid.

### Step 2

N-[2-hydroxy-1-[2-oxo-2-(tritylamino)ethyl]-5-phenyl-pentyl]octanamide (800 mg, 1.32 mmol, 1 eq) was purified by SFC (column: DAICEL CHIRALCEL OD(250 mm*30 mm, 10 um); mobile phase: [0.1% NH₃H₂O ETOH]; B %: 45%-45%, 15 min) to give compound 3A N-[(1R,2S)-2-hydroxy-1-[2-oxo-2-(tritylamino)ethyl]-5-phenyl-pentyl]octanamide (80 mg, 132.27 umol, 10.00% yield, 100% purity) as a white solid.

Compound 3B N-[(1R,2R)-2-hydroxy-1-[2-oxo-2-(tritylamino)ethyl]-5-phenyl-pentyl]octanamide (170 mg, 281.08 umol, 21.25% yield, 100% purity) as a white solid.

Compound 3C N-[(1S,2S)-2-hydroxy-1-[2-oxo-2-(tritylamino)ethyl]-5-phenyl-pentyl]octanamide (200 mg, 330.68 umol, 25.00% yield, 100% purity) as a white solid.

Compound 3D N-[(1S,2R)-2-hydroxy-1-[2-oxo-2-(tritylamino)ethyl]-5-phenyl-pentyl]octanamide (110 mg, 174.60 umol, 13.20% yield, 96% purity) as a white solid.

### Step 3

The solution of N-[(1R,2S)-2-hydroxy-1-[2-oxo-2-(tritylamino)ethyl]-5-phenyl-pentyl]octanamide (80 mg, 132.27 umol, 1 eq), TES (0.05 mL), H₂O (0.05 mL), DCM (0.1 mL) and TFA (3.6 mL) was stirred at 25° C. for 1 hr. LCMS showed the starting reactant was consumed and have the desired mass. The mixture was concentrated under reduced pressure to give a residue. The residue was purified by prep-HPLC (column: Phenomenex Luna C18 100*30 mm*5 um; mobile phase: [water(0.1% TFA)-ACN]; B %: 45%-75%, 10 min) to give N-[(1R,2S)-1-(2-amino-2-oxo-ethyl)-2-hydroxy-5-phenyl-pentyl]octanamide (3.6 mg, 9.93 umol, 7.51% yield) as a white solid.

### Step 4

The solution of N-[(1S,2R)-2-hydroxy-1-[2-oxo-2-(tritylamino)ethyl]-5-phenyl-pentyl]octanamide (110 mg, 181.87 umol, 1 eq), TES (0.05 mL), H₂O (0.05 mL), DCM (0.1 mL) and TFA (3.6 mL) was stirred at 25° C. for 1 hr. The mixture was concentrated under reduced pressure to give a residue. The residue was purified by prep-HPLC (column: Phenomenex Luna C18 100*30 mm*5 um; mobile phase: [water(0.1% TFA)-ACN]; B %: 45%-75%, 10 min) to give Compound N-[(1S,2R)-1-(2-amino-2-oxo-ethyl)-2-hydroxy-5-phenyl-pentyl] octanamide (1.3 mg, 2.94 umol, 1.62% yield, 82% purity) as a white solid.

### Example 95: (3R)-3-(3-hexylureido)-4-hydroxy-7-phenylheptanamide

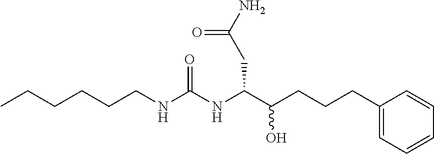 [SMILES: CCCCCCNC(=O)N[C@H](CC(N)=O)C(O)CCCc1ccccc1]

To be prepared according to procedure for compound 94, replacing octanoic anhydride with n-hexylamine and 1,1′-carbonyldiimidazole in step-3.

### Example 96: Hexyl ((3R)-1-amino-4-hydroxy-1-oxo-7-phenylheptan-3-yl)carbamate

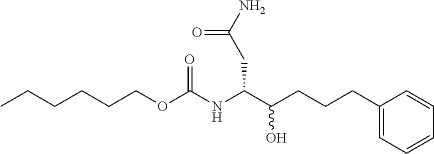 [SMILES: CCCCCCOC(=O)N[C@H](CC(N)=O)C(O)CCCc1ccccc1]

To be prepared according to procedure for compound 94, replacing octanoic anhydride with n-hexanol and 1,1′-carbonyldiimidazole in step-3.

### Example 97: N-((3R)-1-amino-4-hydroxy-6,6-dimethyl-1-oxoheptan-3-yl)octanamide

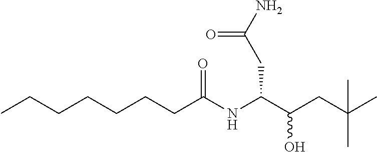 [SMILES: CCCCCCCC(=O)N[C@H](CC(N)=O)C(O)CC(C)(C)C]

To be prepared according to procedure for compound 94, replacing tert-butyl (R)-(1,4-dioxo-7-phenyl-1-(tritylamino)heptan-3-yl)carbamate with tert-butyl (R)-(6,6-dimethyl-1,4-dioxo-1-(tritylamino)heptan-3-yl)carbamate in step-1.

### Example 98: (R)-N-(1-amino-1,4-dioxo-7-phenylheptan-3-yl)octanamide

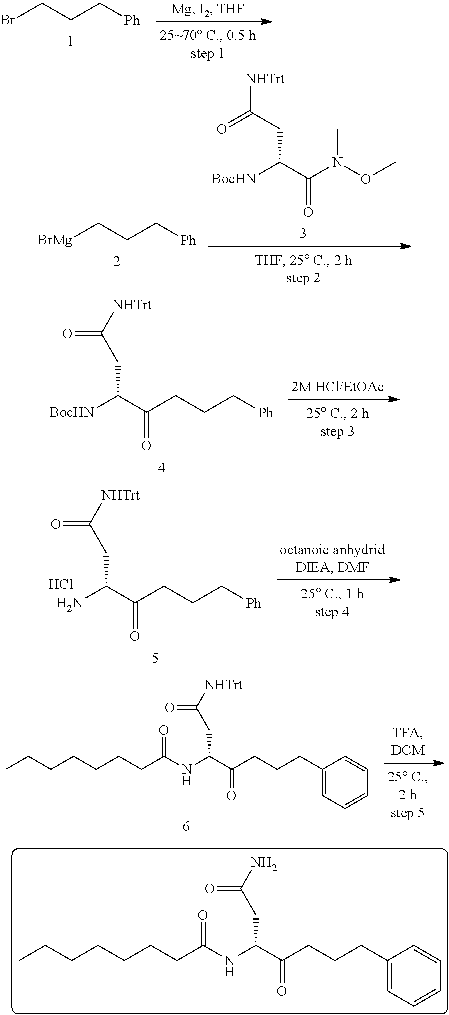

### Step 1

To a solution of Mg (3.05 g, 125.57 mmol, 5 eq) and molecular iodine (20 mg, 78.80 umol, 15.87 uL, 3.14e-3 eq) in THF (50 mL) was added 3-bromopropylbenzene (5 g, 25.11 mmol, 3.79 mL, 1 eq) at 25° C. The mixture was stirred at 70° C. for 0.5 hr. TLC showed the starting reactant was consumed and one new spot was formed. Compound bromo(3-phenylpropyl)magnesium (5.6 g, crude) as a colorless liquid was used into the next step.

### Step 2

To a solution of tert-butyl N-[(1R)-1-[methoxy(methyl)carbamoyl]-3-oxo-3-(tritylamino) propyl]carbamate (1 g, 1.93 mmol, 1 eq) and bromo(3-phenylpropyl)magnesium (4.32 g, 19.32 mmol, 10 eq) in THF (10 mL). The mixture was stirred at 25° C. for 2 hr. TLC showed most starting reactant was consumed and one new spot was formed. The reaction mixture was quenched by addition H₂O 10 mL at 0° C. and extracted with EtOAc 30 mL(10 mL*3). The combined organic layers were washed with saturated brine 30 mL (10 mL*3), dried over Na₂SO₄, filtered and concentrated under reduced pressure to give a residue. The residue was purified by column chromatography (SiO₂, Petroleum ether/Ethyl acetate=30/1 to 5/1) to give compound tert-butyl N-[(1R)-2-oxo-1-[2-oxo-2-(tritylamino)ethyl]-5-phenyl-pentyl]carbamate (856 mg, 1.48 mmol, 76.83% yield) as a white solid.

### Step 3

To a solution of tert-butyl N-[(1R)-2-oxo-1-[2-oxo-2-(tritylamino)ethyl]-5-phenyl-pentyl] carbamate (800 mg, 1.39 mmol, 1 eq) in EtOAc (5 mL) was added HCl/EtOAc (4 M, 6.67 mL, 19.22 eq). The mixture was stirred at 25° C. for 2 hr. TLC showed the starting reactant was consumed and one new spot was formed. The mixture was concentrated under reduced pressure to give a residue to give compound (3R)-3-amino-4-oxo-7-phenyl-N-trityl-heptanamide (670 mg, crude, HCl) as a white solid.

### Step 4

To a solution of (3R)-3-amino-4-oxo-7-phenyl-N-trityl-heptanamide (670 mg, 1.31 mmol, 1 eq, HCl) and octanoyl octanoate (529.67 mg, 1.96 mmol, 1.5 eq) in DMF (10 mL) was added DIPEA (506.32 mg, 3.92 mmol, 682.38 uL, 3 eq). The mixture was stirred at 25° C. for 1 hr. TLC showed one new spot was formed. The reaction mixture was quenched by addition H₂O 10 mL at 0° C. and extracted with EtOAc 30 mL (10 mL*3). The combined organic layers were washed with saturated brine 30 mL (10 mL*3), dried over Na₂SO₄, filtered and concentrated under reduced pressure to give a residue. The residue was purified by column chromatography (SiO₂, Petroleum ether/Ethyl acetate=30/1 to 3/1) to give compound N-[(1R)-2-oxo-1-[2-oxo-2-(tritylamino)ethyl]-5-phenyl-pentyl]octanamide (600 mg, 995.35 umol, 76.22% yield) as a white solid.

### Step 5

To a solution of N-[2-oxo-1-[2-oxo-2-(tritylamino)ethyl]-5-phenyl-pentyl]octanamide (500 mg, 829.46 umol, 1 eq) in DCM (5 mL) was added TFA (1 mL). The mixture was stirred at 25° C. for 2 hr. The mixture was concentrated under reduced pressure to give a residue. The residue was purified by prep-HPLC (column: Phenomenex Luna C18 100*30 mm*5 um; mobile phase: [water(0.1% TFA)-ACN]; B %: 40%-70%, 10 min) to give compound N-[1-(2-amino-2-oxo-ethyl)-2-oxo-5-phenyl-pentyl]octanamide (13 mg, 36.06 umol, 4.35% yield, 100% purity) as a white solid and compound N-[5-hydroxy-2-(3-phenylpropyl)-1H-pyrrol-3-yl]octanamide (24 mg, 70.08 umol, 8.45% yield, 100% purity) as a light yellow solid.

### Example 99: (R)-3-(3-hexylureido)-4-oxo-7-phenylheptanamide

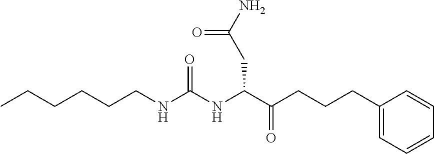 [SMILES: CCCCCCNC(=O)N[C@H](CC(N)=O)C(=O)CCCc1ccccc1]

To be prepared according to procedure for compound 98, starting from step-2 instead of step-1 with tert-butyl (R)-(1,4-dioxo-7-phenyl-1-(tritylamino)heptan-3-yl)carbamate and in step-3 replacing octanoic anhydride with n-hexylamine and 1,1′-carbonyldiimidazole.

### Example 100: hexyl (R)-(1-amino-1,4-dioxo-7-phenylheptan-3-yl)carbamate

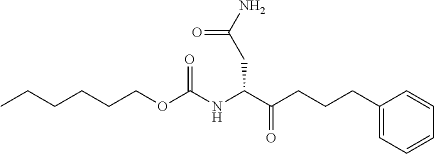 [SMILES: CCCCCCOC(=O)N[C@H](CC(N)=O)C(=O)CCCc1ccccc1]

To be prepared according to procedure for compound 98, starting from step-2 instead of step-1 with tert-butyl (R)-(1,4-dioxo-7-phenyl-1-(tritylamino)heptan-3-yl)carbamate and in step-3 replacing octanoic anhydride with n-hexanol and 1,1′-carbonyldiimidazole.

### Example 101: (R)-N-(1-amino-1,4-dioxo-7-phenylheptan-3-yl)octanamide

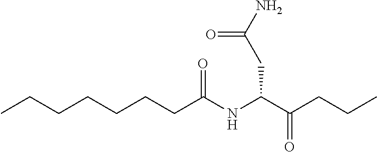 [SMILES: CCCCCCCC(=O)N[C@H](CC(N)=O)C(=O)CCC]

To be prepared according to procedure for compound 98, starting from step-2 instead of step-1 with tert-butyl (R)-(1,4-dioxo-1-(tritylamino)heptan-3-yl)carbamate.

### Example 102: (R)-7,7-dimethyl-3-octanamido-4-oxooctanamide

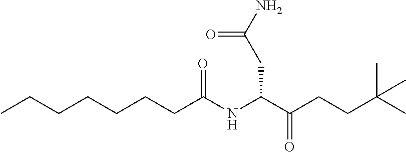 [SMILES: CCCCCCCC(=O)N[C@H](CC(N)=O)C(=O)CCC(C)(C)C]

To be prepared according to procedure for compound 98, starting from step-2 instead of step-1 with tert-butyl (R)-(7,7-dimethyl-1,4-dioxo-1-(tritylamino)octan-3-yl)carbamate.

### Example 103: (S)-(3-amino-1-octanamido-3-oxopropyl)boronic acid

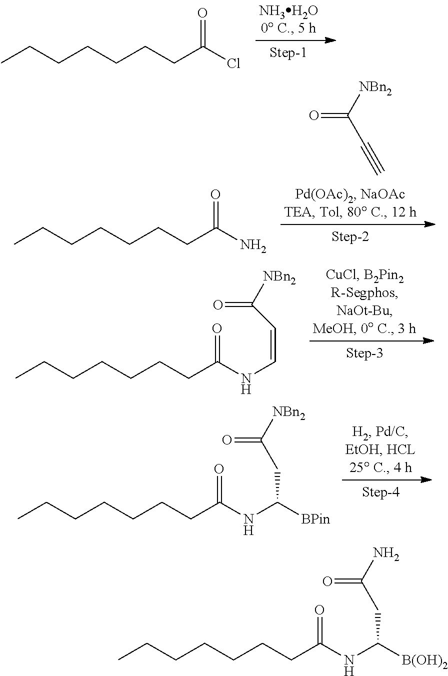

### Step 1

Octanoyl chloride is treated with 1 M solution of ammonia at 0° C. Upon reaction completion the mixture is extracted with ethyl acetate. The solvents are removed in vacuo and the amide is purified by normal phase flash chromatography with a suitable solvent system to give octanamide.

### Step 2

To a solution of octanamide (1 mM) in toluene is added 3 eq. triethylamine, Pd(OAc)₂ (catalytic amounts), 1 eq. NaOAc and N,N-dibenzylpropiolamide under an argon atmosphere. The reaction was heated to 80° C. After all starting material is consumed the mixture is extracted with ethyl acetate, purified by normal phase flash column chromatography to give (Z)-N-(3-(dibenzylamino)-3-oxoprop-1-en-1-yl)octanamide.

### Step 3

To a solution of (Z)-N-(3-(dibenzylamino)-3-oxoprop-1-en-1-yl)octanamide (1 mM) in methanol is treated with CuCl, bis(pinacolato)diboron, R-Segphos and NaO^{t}Bu at 0° C. Upon completion the reaction is worked up and then purified via column chromatography with a suitable solvents to give the (S)-N-(3-(dibenzylamino)-3-oxo-1-(4,4,5,5-tetramethyl-1,3,2-dioxaborolan-2-yl)propyl)octanamide.

### Step 4

To a solution of (S)-N-(3-(dibenzylamino)-3-oxo-1-(4,4,5,5-tetramethyl-1,3,2-dioxaborolan-2-yl)propyl)octanamide (1 mM) in ethanol is treated with palladium on carbon catalyst and hydrochloric acid (5 eq). The mixture is placed under a hydrogen balloon and stirred until reaction is completed. The catalyst is filtered off through a plug of celite. The hydrochloride salt is isolated via reverse phase column chromatography from suitable solvents to produce the desired (S)-(3-amino-1-octanamido-3-oxopropyl)boronic acid.

### Example 104: (S)-(3-amino-1-(3-hexylureido)-3-oxopropyl)boronic acid

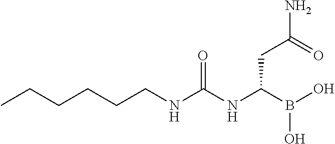 [SMILES: CCCCCCNC(=O)N[C@H](CC(N)=O)B(O)O]

To be prepared according to procedure for compound 103, starting from step 2 replacing octanamide with 1-hexylurea.

### Example 105: (((hexyloxy)carbonyl)amino)-3-oxopropyl)boronic acid

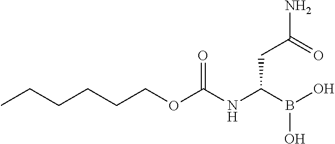 [SMILES: CCCCCCOC(=O)N[C@H](CC(N)=O)B(O)O]

To be prepared according to procedure for compound 103, starting from step 2 replacing octanamide with hexyl carbamate.

### Example 106: (R)-N-(1-amino-1,4-dioxo-5-phenoxypentan-3yl)octanamide

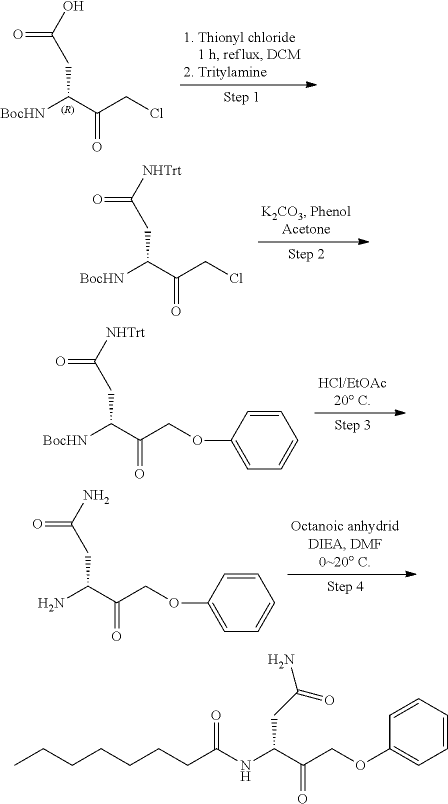

### Step 1

Commercially available (R)-3-((tert-butoxycarbonyl)amino)-5-chloro-4-oxopentanoic acid is dissolve in DCM (0.5 M) and treated with thionyl chloride (2 eq) and reflux for 1 h. After cooling to room temperature 1 eq of tritylamine is added slowly. After completion reaction is worked-up and then purified via column chromatography with a suitable solvents to give the desired tert-butyl (R)-(5-chloro-1,4-dioxo-1-(tritylamino)pentan-3-yl)carbamate.

### Step 2

To a solution of tert-butyl (R)-(5-chloro-1,4-dioxo-1-(tritylamino)pentan-3-yl)carbamate (0.5 M) in acetone is added 3 eq phenol followed by 5 eq of potassium carbonate. After all starting material is consumed the mixture is extracted with ethyl acetate, purified by normal phase flash column chromatography to give tert-butyl (R)-(1,4-dioxo-5-phenoxy-1-(tritylamino)pentan-3-yl)carbamate.

### Step 3

To a solution of tert-butyl (R)-(1,4-dioxo-5-phenoxy-1-(tritylamino)pentan-3-yl)carbamate (0.5 M) in dry ethyl acetate is added HCl in EtOAc under an argon atmosphere. After all starting material is consumed the mixture is extracted with ethyl acetate, purified by reverse phase flash column chromatography to produce the (R)-3-amino-4-oxo-5-phenoxypentanamide.

### Step 4

To a solution of (R)-3-amino-4-oxo-5-phenoxypentanamide (1 mM) in DMF and 5 eq of DIEA at room temperature is added 3 eq of octanoic anhydride. Upon completion of the reaction is worked up and then purified via column chromatography from suitable solvents to produce the desired (R)-N-(1-amino-1,4-dioxo-5-phenoxypentan-3-yl)octanamide.

### Example 107: (R)-N-(1-amino-5-(2,6-difluorophenoxy)-1,4-dioxopentan-3-yl)octanamide

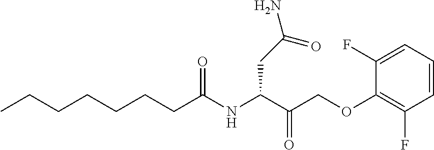 [SMILES: CCCCCCCC(=O)N[C@H](CC(N)=O)C(=O)COc1c(F)cccc1F]

To be prepared according to procedure for compound 106, replacing phenol in step-2 with 2,6-difluorophenol.

### Example 108: (R)-N-(1-amino-5-(2-chlorophenoxy)-1,4-dioxopentan-3-yl)octanamide

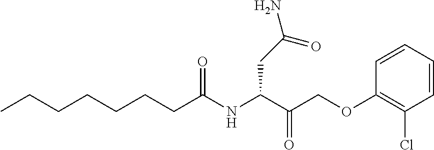 [SMILES: CCCCCCCC(=O)N[C@H](CC(N)=O)C(=O)COc1ccccc1Cl]

To be prepared according to procedure for compound 106, replacing phenol in step-2 with 2-chlorophenol.

### Example 109: (R)-N-(1-amino-1,4-dioxo-5-(o-tolyloxy)pentan-3-yl)octanamide

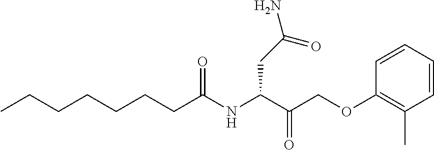 [SMILES: CCCCCCCC(=O)N[C@H](CC(N)=O)C(=O)COc1ccccc1C]

To be prepared according to procedure for Example 106, replacing phenol in step-2 with 2-methylphenol.

### Example 110: hexyl (R)-(1-amino-1,4-dioxo-5-phenoxypentan-3-yl)carbamate

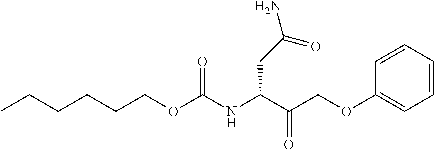 [SMILES: CCCCCCOC(=O)N[C@H](CC(N)=O)C(=O)COc1ccccc1]

To be prepared according to procedure for compound 106, replacing octanoic anhydride with n-hexanol and 1,1′-carbonyldiimidazole in step-4.

### Example 111: (R)-3-(3-hexylureido)-4-oxo-5-phenoxypentanamide

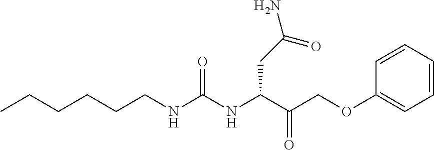 [SMILES: CCCCCCNC(=O)N[C@H](CC(N)=O)C(=O)COc1ccccc1]

To be prepared according to procedure for compound 106, replacing octanoic anhydride with n-hexylamine and 1,1′-carbonyldiimidazole in step-4.

### Example 112: (R)-N-(4-amino-1,4-dioxo-1-phenylbutan-2-yl)octanamide

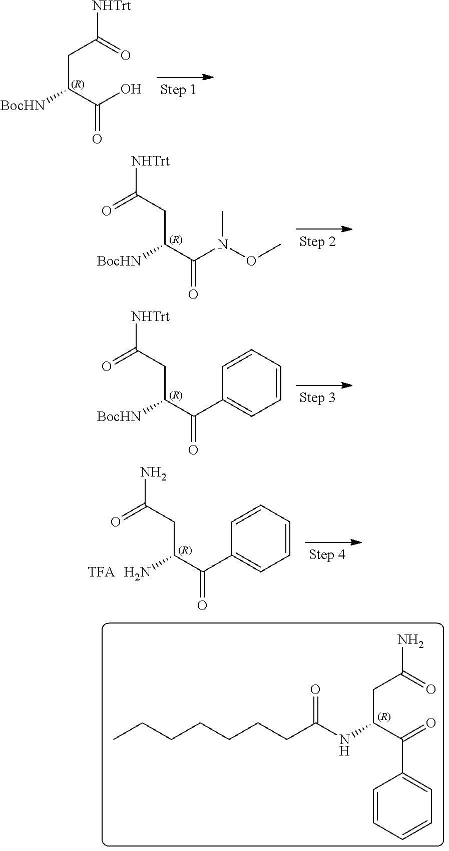

### Step 1: Synthesis of tert-butyl N-[(2R)-1-oxo-1-phenyl-3-[(triphenylmethyl)carbamoyl]-propan-2-yl]carbamate

tert-Butyl N-[(1R)-1-[methoxy(methyl)carbamoyl]-2-[(triphenylmethyl)carbamoyl]ethyl]carbamate (980 mg, 1.89 mmol, 1.00 eq) was dissolved in anhydrous THF (15 mL) and cooled to −78° C. under inert atmosphere. Phenyllithium (1.9 M in dibutyl ether, 3.19 mL, 6.06 mmol, 3.2 eq) was slowly added dropwise. The reaction was continued at −78° C. for 1 h and monitored by TLC (33% AcOEt in Hexane)/LC-MS. Upon completion of the reaction, the temperature was increased up to 0° C. and water was added slowly. The mixture was diluted with AcOEt and extracted. Organic layer was dried over anhydrous Na₂SO₄, filtered and concentrated. The residue was triturated with Et₂O and filtered. The filtrate was disposed. Solids were dried thoroughly to afford the title compound as a white solid (650 mg, purity: ˜50%).

### Step 2: Synthesis of (3R)-3-amino-4-oxo-4-phenylbutanamide

To a solution of tert-butyl N-[(2R)-1-oxo-1-phenyl-3-[(triphenylmethyl)carbamoyl]propan-2-yl]carbamate (700 mg, 1.309 mmol, 1.00 eq) in DCM (10 mL), water (2.5 mL) was added. The mixture was stirred vigorously with trifluoroacetic acid (10 mL, 131 mmol, 100 eq) being added dropwise. The reaction was carried out for 24 h at room temperature. Solvents were removed in vacuo. The residue was redissolved in DCM and extracted with water. Organic layer was disposed. Aqueous layer was separated, concentrated in vacuo and lyophilized until dryness to afford the title compound as trifluoroacetic salt (360 mg, purity: ˜50%)

### Step 3: Synthesis of N-[(2R)-3-carbamoyl-1-oxo-1-phenylpropan-2-yl]octanamide

(3R)-3-amino-4-oxo-4-phenylbutanamide trifluoroacetic acid salt (350 mg, 1.143 mmol, 1.00 eq) was dissolved in anhydrous DMF (5 mL). N,N-diisopropylethylamine (DIPEA, 597 μL, 3.429 mmol, 3.00 eq) was added, followed by dropwise addition of n-caprylic anhydride (374 μL, 1.257 mmol, 1.1 eq). The reaction was carried out at room temperature for 2 h until substrate was fully consumed. Reaction mixture was then diluted in AcOEt, washed subsequently with 0.01M HCl, 1M NaOH and brine. Organic layer was dried over Na₂SO₄, filtered and concentrated. The residue was purified by prep-HPLC to afford the title compound as a white solid (35 mg, 9%).

### Example 113: (R)-N-(4-amino-1-(2,6-difluorophenyl)-1,4-dioxobutan-2-yl)octanamide

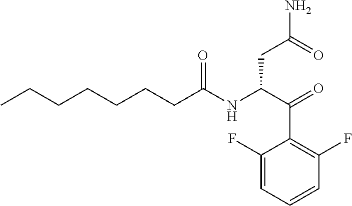 [SMILES: CCCCCCCC(=O)N[C@H](CC(N)=O)C(=O)c1c(F)cccc1F]

To be prepared according to procedure for compound 112, replacing phenyllithium in step-2, with (2,6-difluorophenyl)lithium.

### Example 114: (R)-N-(4-amino-1-(2-chlorophenyl)-1,4-dioxobutan-2-yl)octanamide

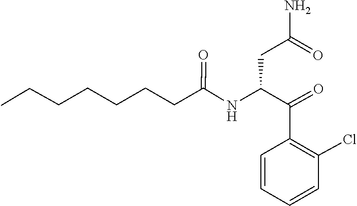 [SMILES: CCCCCCCC(=O)N[C@H](CC(N)=O)C(=O)c1ccccc1Cl]

To be prepared according to procedure for compound 112, replacing phenyllithium in step-2, with (2-chlorophenyl)lithium.

### Example 115: (R)-N-(4-amino-1,4-dioxo-1-(o-tolyl)butan-2-yl)octanamide

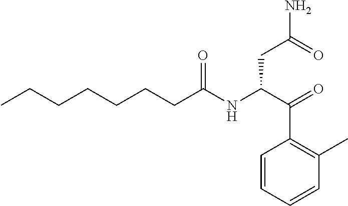 [SMILES: CCCCCCCC(=O)N[C@H](CC(N)=O)C(=O)c1ccccc1C]

To be prepared according to procedure for compound 112, replacing phenyllithium in step-2, with o-tolyllithium.

### Example 116: hexyl (R)-(4-amino-1,4-dioxo-1-phenylbutan-2-yl)carbamate

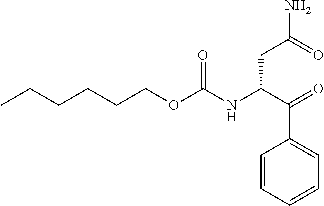 [SMILES: CCCCCCOC(=O)N[C@H](CC(N)=O)C(=O)c1ccccc1]

To be prepared according to procedure for compound 112, replacing octanoic anhydride with n-hexanol and 1,1′-carbonyldiimidazole in step-4.

### Example 117: (R)-3-(3-hexylureido)-4-oxo-4-phenylbutanamide

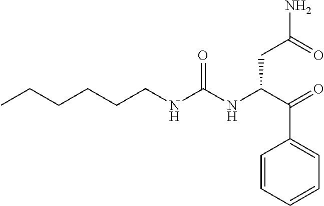 [SMILES: CCCCCCNC(=O)N[C@H](CC(N)=O)C(=O)c1ccccc1]

To be prepared according to procedure for compound 112, replacing octanoic anhydride with n-hexylamine and 1,1′-carbonyldiimidazole in step-4.

### Example 118: (R)-N-(4-amino-1-(oxazol-2-yl)-1,4-dioxobutan-2-yl)octanamide

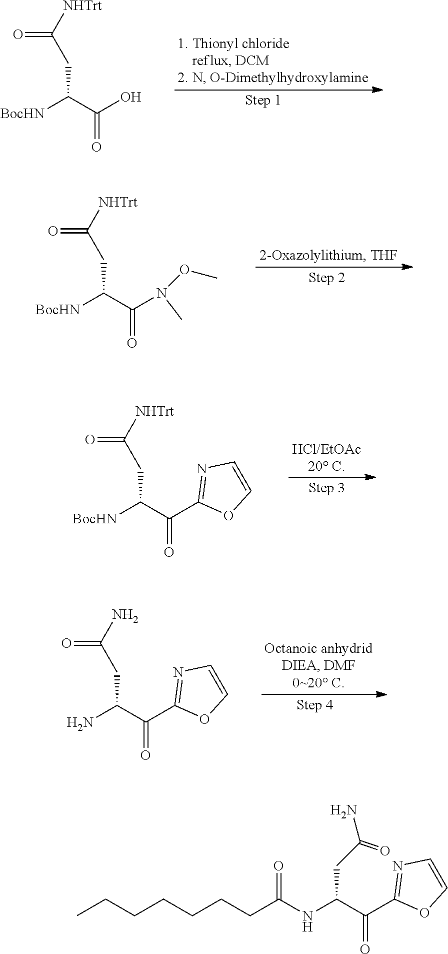

### Step 1

Commercially available N2-(tert-butoxycarbonyl)-N4-trityl-D-asparagine is dissolve in DCM (0.5 M) and treated with thionyl chloride (2 eq) and reflux for 1 h. After cooling to room temperature 1 eq of N,O-dimethylhydroxylamine added slowly. After completion, reaction is worked-up and then purified via column chromatography with a suitable solvents to give the desired tert-butyl (R)-(1-(methoxy(methyl)amino)-1,4-dioxo-4-(tritylamino)butan-2-yl)carbamate.

### Step 2

To a solution of tert-butyl (R)-(1-(methoxy(methyl)amino)-1,4-dioxo-4-(tritylamino)butan-2-yl)carbamate (0.5 M) in THF is added 2 eq 2-Oxazolyllithium slowly at 0° C. After all starting material is consumed the reaction is quench with sat NH₄Cl and the mixture is extracted with ethyl acetate, purified by normal phase flash column chromatography to give tert-butyl (R)-(1-(oxazol-2-yl)-1,4-dioxo-4-(tritylamino)butan-2-yl)carbamate.

### Step 3

To a solution of tert-butyl (R)-(1-(oxazol-2-yl)-1,4-dioxo-4-(tritylamino)butan-2-yl)carbamate (0.5 M) in dry ethyl acetate is added HCl/in EtOAc (catalytic amounts) under an argon atmosphere. After all the starting material is consumed the mixture is extracted with ethyl acetate, purified by reverse phase flash column chromatography to produce the (R)-3-amino-4-(oxazol-2-yl)-4-oxobutanamide.

### Step 4

To a solution of (R)-3-amino-4-oxo-4-phenylbutanamide(R)-3-amino-4-(oxazol-2-yl)-4-oxobutanamide (1 mM) in DMF and 5 eq of DIEA at room temperature is added 3 eq of octanoic anhydride. Upon completion of the reaction, it is worked-up and then purified via column chromatography from suitable solvents to produce the desired (R)-N-(4-amino-1,4-dioxo-1-phenylbutan-2-yl)octanamide.

### Example 119: hexyl (R)-(4-amino-1-(oxazol-2-yl)-1,4-dioxobutan-2-yl)carbamate

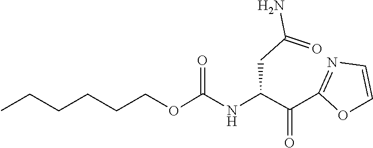 [SMILES: CCCCCCOC(=O)N[C@H](CC(N)=O)C(=O)c1ncco1]

To be prepared according to procedure for compound 118, replacing octanoic anhydride with n-hexanol and 1,1′-carbonyldiimidazole in step-4.

### Example 120: (R)-3-(3-hexylureido)-4-(oxazol-2-yl)-4-oxobutanamide

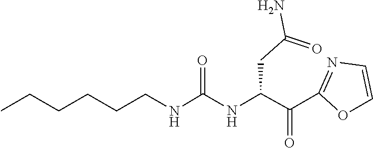 [SMILES: CCCCCCNC(=O)N[C@H](CC(N)=O)C(=O)c1ncco1]

To be prepared according to procedure for compound 118, replacing octanoic anhydride with n-hexylamine and 1,1′-carbonyldiimidazole in step-4.

### Example 121: (R)-N-(4-amino-1-(1H-imidazol-2-yl)-1,4-dioxobutan-2-yl)octanamide

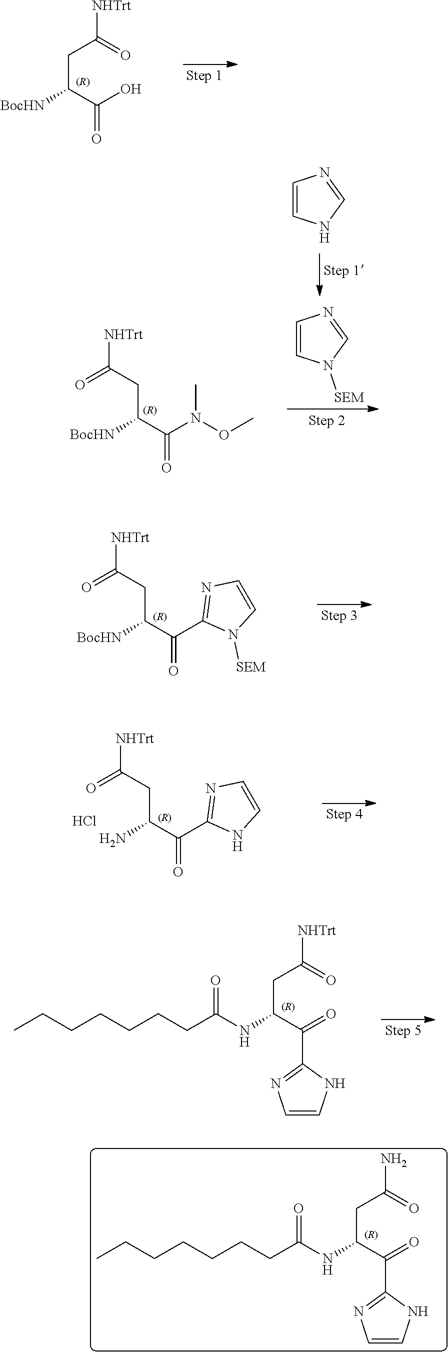

### Step 1: Synthesis of 1-{[2-(trimethylsilyl)ethoxy]methyl}-1H-imidazole

60% NaH (60% mineral oil, 0.881 g, 22.0 mmol, 1.5 eq) was added to a solution of imidazole (1.00 g, 14.7 mmol, 1.0 eq) in DMF (10 mL) at 0° C. and stirred for 1 h. Then, SEM-Cl (3.09 mL, 17.6 mmol, 1.2 eq) was added dropwise at 0° C. and the reaction was warmed to room temperature and stirred for 24 h. The reaction was quenched with a solution of NaHCO₃ sat. and extracted with AcOEt. The organic layer was washed with water, dried over Na₂SO₄, filtered and concentrated under reduced pressure. The crude was purified by flash column chromatography (5% of MeOH in DCM) to give 1-{[2-(trimethylsilyl)ethoxy]methyl}-1H-imidazole as a yellow oil (2.06 g, 69%).

¹H NMR (300 MHz, DMSO-d₆) δ 7.77 (t, J=1.1 Hz, 1H), 7.26 (t, J=1.3 Hz, 1H), 6.93 (t, J=1.1 Hz, 1H), 5.32 (s, 2H), 3.51-3.39 (m, 2H), 0.89-0.77 (m, 2H), −0.04 (s, 9H).

### Step 1: Synthesis of tert-butyl N-[(1R)-1-[methoxy(methyl)carbamoyl]-2-[(triphenylmethyl)carbamoyl]ethyl]carbamate

To a solution of (2R)-2-{[(tert-butoxy)carbonyl]amino}-3-(triphenylmethyl)carbamoyl]propanoic acid (1.00 g, 2.107 mmol, 1.00 eq) and 1-hydroxybenzotriazole monohydrate (323 mg, 2.11 mmol, 1.00 eq) in anhydrous DMF (11 mL), diisopropylcarbodiimide (DIC, 326 μL, 2.107 mmol, 1.00 eq) was added dropwise. The mixture was stirred at room temperature for 20 min and then followed by the addition of N,N-diisopropylethylamine (DIPEA, 367 μL, 2.107 mmol, 1.00 eq) and N,O-dimethylhydroxylamine hydrochloride (206 mg, 2.107 mmol, 1.00 eq) in one portion. The reaction was continued for 24 h and monitored by TLC (35-50% AcOEt in hexane) and LC-MS. Upon completion, the mixture was diluted with AcOEt, washed with water, sat. NaHCO₃ and brine. The organic layer was dried over anhydrous Na₂SO₄, filtered and concentrated. Although the product contained ˜10% of diisopropyl urea, it was used in the next step without further purification. Product as a white solid (1.00 g, 83%).

¹H NMR (300 MHz, Chloroform-d) 67.33-7.27 (m, 4H), 7.25-7.14 (m, 11H), 5.64 (d, J=8.7 Hz, 1H), 4.96 (dd, J=6.7 Hz, 1H), 3.69 (s, 3H), 3.14 (s, 3H), 2.82-2.55 (m, 2H), 1.40 (s, 9H).

### Step 2: Synthesis of tert-butyl N-[(2R)-1-oxo-1-(1-{[2-(trimethylsilyl)ethoxy]methyl}-1H-imidazol-2-yl)-3-[(triphenylmethyl)carbamoyl]propan-2-yl]carbamate

n-BuLi solution (1.6 M in hexanes, 4.41 mL, 7.05 mmol, 7.3 eq) was added dropwise to a solution of 1-{[2-(trimethylsilyl)ethoxy]methyl}-1H-imidazole (0.958 g, 4.83 mmol, 5.0 eq) in THF (10 mL) at −78° C. After 30 min, a solution of tert-butyl N-[(1R)-1-[methoxy(methyl)carbamoyl]-2-[(triphenylmethyl)carbamoyl]ethyl]carbamate (0.500 g, 0.966 mmol, 1.0 eq) in THF (5 mL) was added dropwise and the reaction mixture was stirred for 30 min. Then reaction mixture was slowly quenched with sat. NH₄Cl at 0° C. and partitioned between AcOEt and water. The organic layer was dried over Na₂SO₄, filtered and concentrated in vacuum. The residue was purified by FCC (20-40% EtOAc in hexane) to give the tert-butyl N-[(2R)-1-oxo-1-(1-{[2-(trimethylsilyl)ethoxy]methyl}-1H-imidazol-2-yl)-3-[(triphenylmethyl)carbamoyl]propan-2-yl]carbamate as an colorless oil (285 mg, 43%).

¹H NMR (300 MHz, Chloroform-d) 67.38 (s, 1H), 7.34-7.15 (m, 16H), 7.13 (d, J=1.0 Hz, 1H), 5.76 (s, 1H), 5.75 (d, J=10.4 Hz, 1H), 5.56 (d, J=10.3 Hz, 1H), 5.53-5.44 (m, 1H), 3.62-3.47 (m, 2H), 3.22-3.03 (m, 2H), 1.44 (s, 9H), 0.99-0.84 (m, 2H), −0.01 (s, 9H).

### Step 3: Synthesis of (2R)-1-(1H-imidazol-2-yl)-1-oxo-3-[(triphenylmethyl)carbamoyl]propan-2-aminium chloride

To a solution of tert-butyl N-[(2R)-1-oxo-1-(1-{[2-(trimethylsilyl)ethoxy]methyl}-1H-imidazol-2-yl)-3-[(triphenylmethyl)carbamoyl]propan-2-yl]carbamate (190 mg, 0.29 mmol, 1.0 eq) in dioxane (1 mL) was added 4M HCl in dioxane (1.45 mL, 5.80 mmol, 20 eq) and reaction mixture was stirred at room temperature for 24 h. After this time the solvent was removed under reduced pressure and crude product was used directly in the next step (130 mg, HCl salt, crude).

### Step 4: Synthesis of N-[(2R)-1-(1H-imidazol-2-yl)-1-oxo-3-[(triphenylmethyl)carbamoyl]propan-2-yl]octanamide

n-Octanoic anhydride (62 uL, 0.363 mmol, 1.1 eq) was added to a stirring solution of (2R)-1-(1H-imidazol-2-yl)-1-oxo-3-[(triphenylmethyl)carbamoyl]propan-2-aminium chloride (140 mg, 0.330 mmol, 1.0 eq) and DIPEA (0.172 mL, 0.989 mmol, 3.0 eq) in anhydrous DMF (3 mL). After 24 h, LC-MS analysis confirmed formation of the desired product. The reaction was diluted with DCM and washed with water, brine, dried over Na₂SO₄, filtered and concentrated under reduced pressure. The crude product was used directly in the next step (190 mg, crude).

### Step 5: Synthesis of N-[(2R)-3-carbamoyl-1-(1H-imidazol-2-yl)-1-oxopropan-2-yl]octanamide

To a solution of N-[(2R)-1-(1H-imidazol-2-yl)-1-oxo-3-[(triphenylmethyl)carbamoyl]propan-2-yl]octanamide (180 mg, 0.327 mmol, 1.0 eq) in DCM (1 mL) was added TFA (0.25 mL, 3.27 mmol, 10 eq) and the reaction mixture was stirred at room temperature for 24 h. After 24 h, LC-MS analysis confirmed formation of the desired product. The solvent was removed under reduced pressure and crude product was purified by preparative HPLC to give N-[(2R)-3-carbamoyl-1-(1H-imidazol-2-yl)-1-oxopropan-2-yl]octanamide (21 mg, 20%).

¹H NMR (300 MHz, DMSO-d₆) δ 13.65 (s, 1H), 8.95 (s, 1H), 7.94 (d, J=7.9 Hz, 1H), 7.36 (s, 2H), 7.13 (s, 1H), 4.69-4.56 (m, 1H), 2.48-2.40 (m, 2H), 2.15-1.95 (m, 2H), 1.46-1.33 (m, 2H), 1.30-1.09 (m, 8H), 0.91-0.79 (m, 3H).

### Example 122: hexyl (R)-(4-amino-1-(1H-imidazol-2-yl)-1,4-dioxobutan-2-yl)carbamate

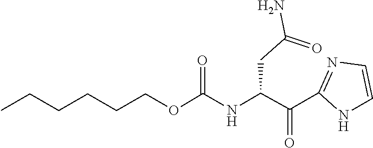 [SMILES: CCCCCCOC(=O)N[C@H](CC(N)=O)C(=O)c1ncc[nH]1]

To be prepared according to procedure for compound 121, replacing octanoic anhydride with n-hexanol and 1,1′-carbonyldiimidazole in step-4.

### Example 123: (R)-3-(3-hexylureido)-4-(1H-imidazol-2-yl)-4-oxobutanamide

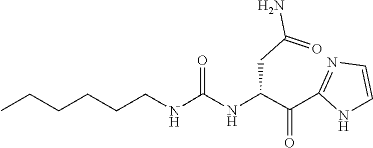 [SMILES: CCCCCCNC(=O)N[C@H](CC(N)=O)C(=O)c1ncc[nH]1]

To be prepared according to procedure for compound 121, replacing octanoic anhydride with n-hexylamine and 1,1′-carbonyldiimidazole in step-4.

### Example 124: (R)-N-(3-amino-3-oxo-1-(2-phenethyl-2H-tetrazol-5-yl)propyl)octanamide

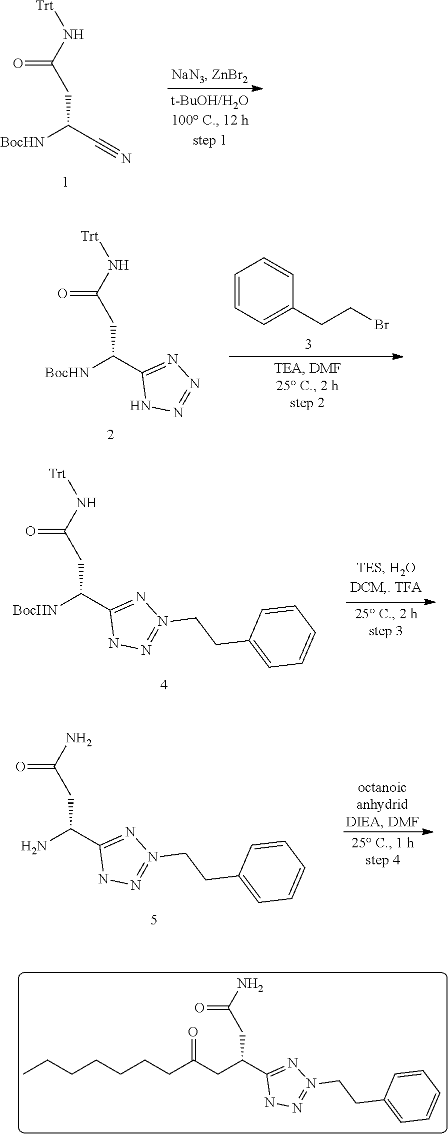

### Step 1

To a solution of tert-butyl N-[(1R)-1-cyano-3-oxo-3-(tritylamino)propyl]carbamate (500 mg, 1.10 mmol, 1 eq) in t-BuOH (9 mL) and H₂O (3 mL) was added NaN₃ (214.06 mg, 3.29 mmol, 3 eq) and ZnBr₂ (271.89 mg, 1.21 mmol, 60.42 uL, 1.1 eq). The mixture was stirred at 100° C. for 12 hrs. TLC showed most starting reactant was consumed and one new spot was formed. The reaction mixture was quenched by addition sat. NaHCO₃ 10 mL at 0° C., extracted with EtOAc 30 mL (10 mL*3). The combined organic layers were washed with saturated brine 30 mL (10 mL*3), dried over Na₂SO₄, filtered and concentrated under reduced pressure to give a residue. The residue was purified by column chromatography (SiO₂, Petroleum ether/Ethyl acetate=30/1 to 1/1) to give compound tert-butyl N-[(1R)-3-oxo-1-(1H-tetrazol-5-yl)-3-(tritylamino)propyl]carbamate (515 mg, 1.03 mmol, 94.11% yield) as a white solid.

### Step 2

To a solution of tert-butyl N-[(1R)-3-oxo-1-(1H-tetrazol-5-yl)-3-(tritylamino)propyl]carbamate (515 mg, 1.03 mmol, 1 eq) in DMF (5 mL) was added TEA (209.05 mg, 2.07 mmol, 287.54 uL, 2 eq) and 2-bromoethylbenzene (382.31 mg, 2.07 mmol, 279.06 uL, 2 eq). The mixture was stirred at 25° C. for 2 hrs. TLC showed the starting reactant was consumed and one new spot was formed. The reaction mixture was quenched by addition H₂O 10 mL at 0° C., extracted with EtOAc 30 mL (10 mL*3). The combined organic layers were washed with saturated brine 30 mL (10 mL*3), dried over Na₂SO₄, filtered and concentrated under reduced pressure to give a residue. The residue was purified by column chromatography (SiO₂, Petroleum ether/Ethyl acetate=30/1 to 2/1) to give compound tert-butyl N-[(1R)-3-oxo-1-[2-(2-phenylethyl)tetrazol-5-yl]-3-(tritylamino) propyl]carbamate (330 mg, 547.51 umol, 53.01% yield) as a white solid.

### Step 3

The mixture of tert-butyl N-[(1R)-3-oxo-1-[2-(2-phenylethyl)tetrazol-5-yl]-3-(tritylamino)propyl]carbamate (300 mg, 497.74 umol, 1 eq), TES (0.1 mL), H₂O (0.1 mL), DCM (0.2 mL) and TFA (3.6 mL) was stirred at 25° C. for 2 hr. The mixture was concentrated under reduced pressure to give compound (3R)-3-amino-3-[2-(2-phenylethyl)tetrazol-5-yl]propanamide(186 mg, crude, TFA) as a white solid.

### Step 4

To a solution of (3R)-3-amino-3-[2-(2-phenylethyl)tetrazol-5-yl]propanamide(186 mg, 496.90 umol, 1 eq, TFA) and octanoyl octanoate (201.55 mg, 745.36 umol, 1.5 eq) in DMF (5 mL) was added DIPEA (192.66 mg, 1.49 mmol, 259.66 uL, 3 eq). The mixture was stirred at 25° C. for 1 hr. The mixture was concentrated under reduced pressure to give a residue. The residue was purified by prep-HPLC (column: Phenomenex Luna C18 100*30 mm*5 um; mobile phase: [water(0.1% TFA)-ACN]; B %: 35%-65%, 10 min) to give compound N-[(1R)-3-amino-3-oxo-1-[2-(2-phenylethyl)tetrazol-5-yl]propyl]octanamide (51 mg, 131.96 umol, 26.56% yield, 100% purity) as a white solid.

### Example 125: (R)-N-(3-amino-1-(2-ethyl-2H-tetrazol-5-yl)-3-oxopropyl)octanamide

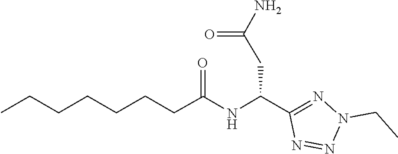 [SMILES: CCCCCCCC(=O)N[C@H](CC(N)=O)c1nnn(CC)n1]

The title compound may be synthesized according to the experimental procedure for Example 124, replacing the (2-bromoethyl)benzene with bromoethane.

### Example 126: (R)-N-(3-amino-1-(2-(tert-butyl)-2H-tetrazol-5-yl)-3-oxopropyl)octanamide

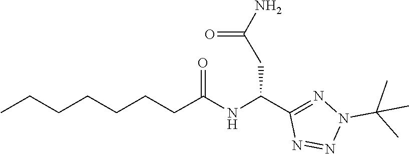 [SMILES: CCCCCCCC(=O)N[C@H](CC(N)=O)c1nnn(C(C)(C)C)n1]

The title compound may be synthesized according to the experimental procedure for Example 124, replacing the (2-bromoethyl)benzene with 2-bromo-2-methylpropane.

### Example 127: (R)-3-(3-hexylureido)-3-(2-phenethyl-2H-tetrazol-5-yl)propenamide

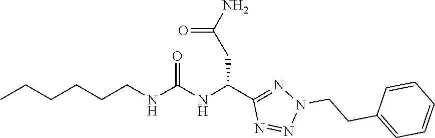 [SMILES: CCCCCCNC(=O)N[C@H](CC(N)=O)c1nnn(CCc2ccccc2)n1]

The title compound may be synthesized according to the experimental procedure for Example 124, replacing the octanoic anhydride with 1-isocyanatohexane.

### Example 128: hexyl (R)-(3-amino-3-oxo-1-(2-phenethyl-2H-tetrazol-5-yl)propyl)carbamate

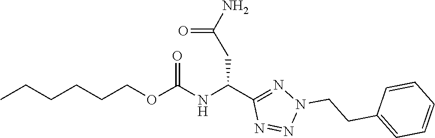 [SMILES: CCCCCCOC(=O)N[C@H](CC(N)=O)c1nnn(CCc2ccccc2)n1]

The title compound may be synthesized according to the experimental procedure for Example 124, replacing the octanoic anhydride with hexyl carbonochloridate.

### Example 129: (R)-N-(3-amino-3-oxo-1-(5-phenethyl-1H-imidazol-2-yl)propyl)octanamide

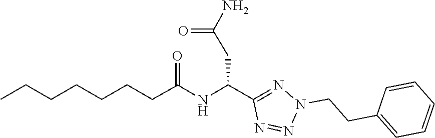 [SMILES: CCCCCCCC(=O)N[C@H](CC(N)=O)c1nnn(CCc2ccccc2)n1]

Step 1: 1-amino-4-phenylbutan-2-one is synthesized by a reaction of phenylpropionic acid with CDI (1 equiv), isocyanoethyl acetate (1.2 equiv), and DBU (3 equiv) in THF. Compound is purified and is then treated with concentrated HCl in methanol to yield 1-amino-4-phenylbutan-2-one.

Step 2: tert-butyl (R)-(1,4-dioxo-4-(tritylamino)butan-2-yl)carbamate is reacted with HATU, 1-amino-4-phenylbutan-2-one, and DIPEA in DMF to give tert-butyl (R)-(1,4-dioxo-1-((2-oxo-4-phenylbutyl)amino)-4-(tritylamino)butan-2-yl)carbamate. A solution of this compound in xylene is treated with excess ammonium acetate and heated to 130° C. for 24 hours. Upon completion, reaction is worked up and purified to yield tert-butyl (R)-(3-oxo-1-(5-phenethyl-1H-imidazol-2-yl)-3-(tritylamino)propyl)carbamate, which is then deprotected with a solution of triethylsilane, dichloromethane, water, and trifluoroacetic acid. The resulting product is treated with octanoic anhydride and DIPEA in DMF at room temperature. Upon completion, reaction is worked up and purified by column chromatography to yield (R)-N-(3-amino-3-oxo-1-(5-phenethyl-1H-imidazol-2-yl)propyl)octanamide.

### Example 130: (R)-N-(3-amino-1-(5-ethyl-1H-imidazol-2-yl)-3-oxopropyl)octanamide

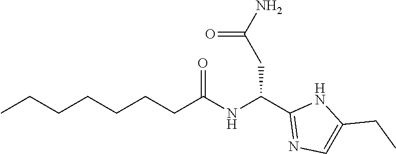 [SMILES: CCCCCCCC(=O)N[C@H](CC(N)=O)c1ncc(CC)[nH]1]

The title compound may be synthesized according to the experimental procedure for Example 129 Step 2, replacing the 1-amino-4-phenylbutan-2-one with 1-aminobutan-2-one.

### Example 131: (R)-N-(3-amino-1-(5-(tert-butyl)-1H-imidazol-2-yl)-3-oxopropyl)octanamide

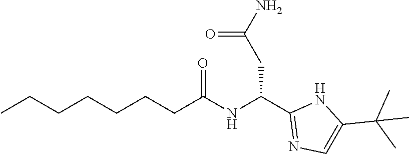 [SMILES: CCCCCCCC(=O)N[C@H](CC(N)=O)c1ncc(C(C)(C)C)[nH]1]

The title compound may be synthesized according to the experimental procedure for Example 129 Step 2, replacing the 1-amino-4-phenylbutan-2-one with 1-amino-3,3-dimethylbutan-2-one.

### Example 132: (R)-3-(3-hexylureido)-3-(5-phenethyl-1H-imidazol-2-yl)propenamide

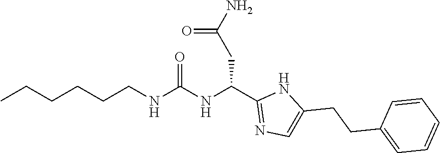 [SMILES: CCCCCCNC(=O)N[C@H](CC(N)=O)c1ncc(CCc2ccccc2)[nH]1]

The title compound may be synthesized according to the experimental procedure for Example 129 Step 2, replacing the octanoic anhydride with 1-isocyanatohexane.

### Example 133: hexyl (R)-(3-amino-3-oxo-1-(5-phenethyl-1H-imidazol-2-yl)propyl)carbamate

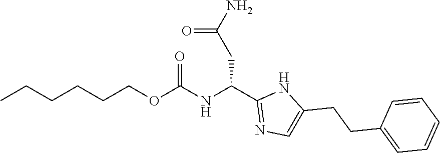 [SMILES: CCCCCCOC(=O)N[C@H](CC(N)=O)c1ncc(CCc2ccccc2)[nH]1]

The title compound may be synthesized according to the experimental procedure for Example 129 Step 2, replacing the octanoic anhydride with hexyl carbonochloridate.

### Example 134: (R)-N-(3-amino-3-oxo-1-(5-phenethyl-4H-1,2,4-triazol-3-yl)propyl)octanamide

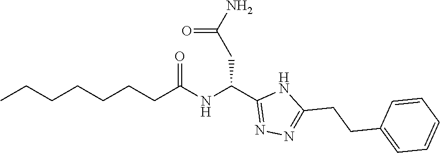 [SMILES: CCCCCCCC(=O)N[C@H](CC(N)=O)c1nnc(CCc2ccccc2)[nH]1]

N²-(tert-butoxycarbonyl)-N⁴-trityl-D-asparagine is reacted with potassium carbonate (1.2 equiv) methyl iodide (2 equiv) in DMF. Upon completion, reaction is worked up and purified to give methyl N²-(tert-butoxycarbonyl)-N⁴-trityl-D-asparaginate, which is then reacted with hydrazine hydrate (10 equiv) in methanol at 50° C. The resulting product tert-butyl (R)-(1-hydrazinyl-1,4-dioxo-4-(tritylamino)butan-2-yl)carbamate is refluxed with sodium methoxide (1 equiv) in methanol followed by the addition of 3-phenylpropanenitrile (1 equiv) to form tert-butyl (R)-(3-oxo-1-(5-phenethyl-4H-1,2,4-triazol-3-yl)-3-(tritylamino)propyl)carbamate. Product is isolated and deprotected with a solution of triethylsilane, dichloromethane, water, and trifluoroacetic acid to give (R)-3-amino-3-(5-phenethyl-4H-1,2,4-triazol-3-yl)propenamide. (R)-3-amino-3-(5-phenethyl-4H-1,2,4-triazol-3-yl)propenamide is then reacted with octanoic anhydride (1.5 equiv) and DIPEA (3 equiv). Upon completion, reaction is worked up and purified by column chromatography to yield (R)-N-(3-amino-3-oxo-1-(5-phenethyl-4H-1,2,4-triazol-3-yl)propyl)octanamide.

### Example 135: (R)-N-(3-amino-1-(5-ethyl-4H-1,2,4-triazol-3-yl)-3-oxopropyl)octanamide

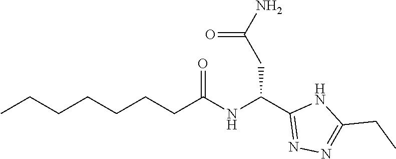 [SMILES: CCCCCCCC(=O)N[C@H](CC(N)=O)c1nnc(CC)[nH]1]

The title compound may be synthesized according to the experimental procedure for Example 134, replacing the 3-phenylpropanenitrile with propiononitrile.

### Example 136: (R)-N-(3-amino-1-(5-(tert-butyl)-4H-1,2,4-triazol-3-yl)-3-oxopropyl)octanamide

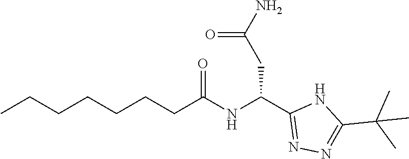 [SMILES: CCCCCCCC(=O)N[C@H](CC(N)=O)c1nnc(C(C)(C)C)[nH]1]

The title compound may be synthesized according to the experimental procedure for Example 134, replacing the 3-phenylpropanenitrile with pivalonitrile.

### Example 137: (R)-3-(3-hexylureido)-3-(5-phenethyl-4H-1,2,4-triazol-3-yl)propenamide

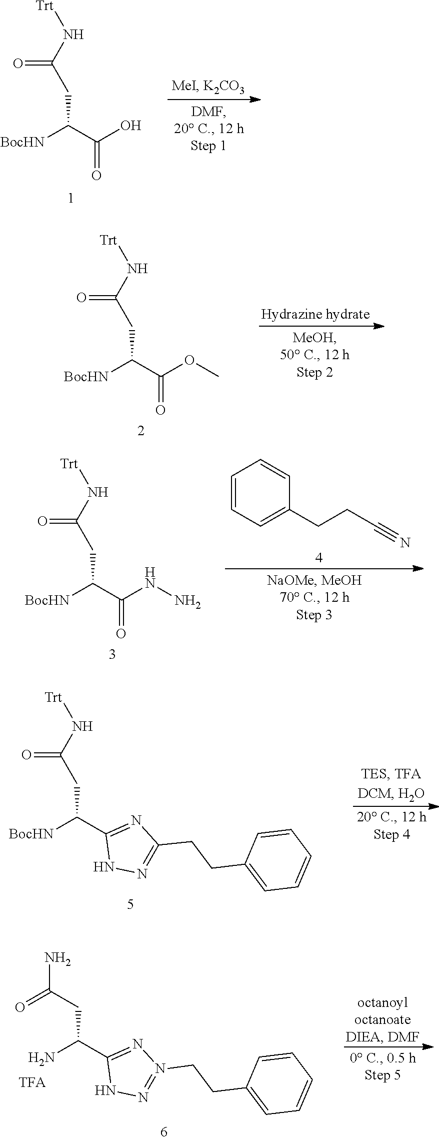

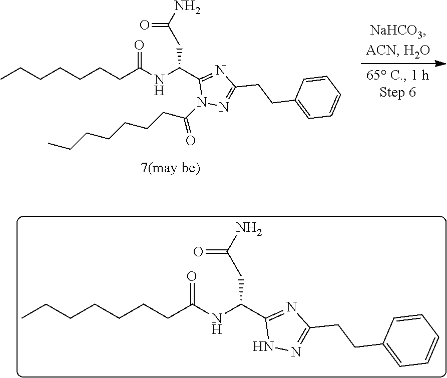 [SMILES: CCCCCCCC(=O)N[C@H](CC(N)=O)c1nc(CCc2ccccc2)nn1C(=O)CCCCCCC ; CCCCCCCC(=O)N[C@H](CC(N)=O)c1nc(CCc2ccccc2)n[nH]1]

### Step 1

To a solution of (2R)-2-(tert-butoxycarbonylamino)-4-oxo-4-(tritylamino)butanoic acid (2 g, 4.21 mmol, 1 eq) in DMF (20 mL) was added K₂CO₃ (873.71 mg, 6.32 mmol, 1.5 eq) and iodomethane (717.85 mg, 5.06 mmol, 314.85 uL, 1.2 eq). The mixture was stirred at 20° C. for 12 hr. TLC indicated reactant was consumed completely. The reaction mixture was poured to water (100 mL) and then the mixture was filtered and the filter cake was concentrated under reduced pressure to give the product methyl (2R)-2-(tert-butoxycarbonylamino)-4-oxo-4-(tritylamino)butanoate (1.7 g, 3.48 mmol, 82.56% yield) as white solid.

### Step 2

To a solution of methyl (2R)-2-(tert-butoxycarbonylamino)-4-oxo-4-(tritylamino)butanoate (1 g, 2.05 mmol, 1 eq) in MeOH (30 mL) was added hydrazine hydrate (627.32 mg, 12.28 mmol, 609.05 uL, 98% purity, 6 eq). The mixture was stirred at 50° C. for 12 h. The reaction mixture was concentrated under reduced pressure to get a residue. The residue was diluted with DCM/MeOH 200 mL and extracted with H₂O 150 mL (50 mL*3). The combined organic layers were washed with brine 100 mL (50 mL*2), dried over Na₂SO₄, filtered and the filtrate was concentrated under reduced pressure to give the product tert-butyl N-[(1R)-1-(hydrazinecarbonyl)-3-oxo-3-(tritylamino)propyl]carbamate (800 mg, 1.60 mmol, 78.40% yield, 98% purity) as white solid.

### Step 3

To a solution of tert-butyl N-[(1R)-1-(hydrazinecarbonyl)-3-oxo-3-(tritylamino)propyl]carbamate (500 mg, 1.02 mmol, 1 eq) in MeOH (10 mL) was added 3-phenylpropanenitrile (268.48 mg, 2.05 mmol, 268.48 uL, 2 eq) and NaOMe (27.64 mg, 511.69 umol, 0.5 eq). The mixture was stirred at 70° C. for 12 hr. The reaction mixture was concentrated under reduced pressure to give a residue. The residue was purified by column chromatography (SiO₂, Petroleum ether/Ethyl acetate=10/1 to 0/1) to give compound tert-butyl N-[(1R)-3-oxo-1-[3-(2-phenylethyl)-1H-1,2,4-triazol-5-yl]-3-(tritylamino)propyl]carbamate (80 mg, 118.32 umol, 11.56% yield, 89% purity) as white solid.

### Step 4

A mixture of triethylsilane (36.40 mg, 313.05 umol, 0.05 mL, 6.28 eq), H₂O (50.00 mg, 2.78 mmol, 0.05 mL, 55.67 eq), DCM (132.00 mg, 1.55 mmol, 0.1 mL, 31.17 eq) and TFA (2.77 g, 24.31 mmol, 1.8 mL, 487.62 eq) was added tert-butyl N-[(1R)-3-oxo-1-[3-(2-phenylethyl)-1H-1,2,4-triazol-5-yl]-3-(tritylamino)propyl]carbamate(30 mg, 49.86 umol, 1 eq). The mixture was stirred at 20° C. for 12 hr under N₂ atmosphere. The reaction mixture was concentrated under reduced pressure to give the product (3R)-3-amino-3-[3-(2-phenylethyl)-1H-1,2,4-triazol-5-yl]propanamide (40 mg, crude) as colorless oil.

### Step 5

To a solution of (3R)-3-amino-3-[3-(2-phenylethyl)-1H-1,2,4-triazol-5-yl]propanamide (40 mg, 107.14 umol, 1 eq, TFA) in DMF (3 mL) was added DIEA (27.69 mg, 214.29 umol, 37.32 uL, 2 eq) and octanoyl octanoate (43.46 mg, 160.72 umol, 1.5 eq). The mixture was stirred at 0° C. for 0.5 hr. LCMS showed the desired compound was detected. The reaction mixture was partitioned between EtOAc 50 mL and H₂O 20 mL. The organic phase was separated, washed with brine 30 mL (10 mL*3), dried over Na₂SO₄, filtered and the filtrate was concentrated under reduced pressure to give the crude product N-[(1R)-3-amino-1-[2-octanoyl-5-(2-phenylethyl)-1,2,4-triazol-3-yl]-3-oxo-propyl]octanamide (50 mg, crude) as brown oil.

### Step 6

A solution of N-[(1R)-3-amino-1-[2-octanoyl-5-(2-phenylethyl)-1,2,4-triazol-3-yl]-3-oxo-propyl]octanamide (50 mg, 97.71 umol, 1 eq) in 4 mL ACN/H₂O (1:1) was added sat. NaHCO₃ (1.08 g, 12.86 mmol, 0.5 mL, 131.57 eq), and stirred at 65° C. for 1 h. The residue was purified by prep-HPLC ([water(10 mM NH₄HCO₃)-ACN]) to give compound N-[(1R)-3-amino-3-oxo-1-[3-(2-phenylethyl)-1H-1,2,4-triazol-5-yl]propyl]octanamide (8 mg, 20.75 umol, 13.33% yield) as white solid.

### Example 138: hexyl (R)-(3-amino-3-oxo-1-(5-phenethyl-4H-1,2,4-triazol-3-yl)propyl)carbamate

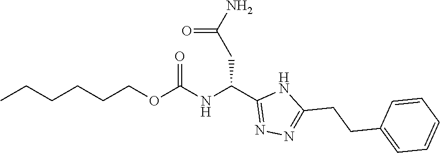 [SMILES: CCCCCCOC(=O)N[C@H](CC(N)=O)c1nnc(CCc2ccccc2)[nH]1]

The title compound may be synthesized according to the experimental procedure for Example 134, replacing the octanoic anhydride with hexyl carbonochloridate.

### Example 139: (R)-N-(3-amino-3-oxo-1-(5-phenethyl-1,3,4-oxadiazol-2-yl)propyl)octanamide

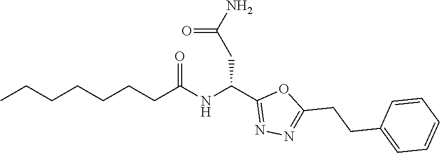 [SMILES: CCCCCCCC(=O)N[C@H](CC(N)=O)c1nnc(CCc2ccccc2)o1]

N²-(tert-butoxycarbonyl)-N⁴-trityl-D-asparagine is reacted with potassium carbonate (1.2 equiv) methyl iodide (2 equiv) in DMF. Upon completion, reaction is worked up and purified to give methyl N²-(tert-butoxycarbonyl)-N⁴-trityl-D-asparaginate, which is then reacted with hydrazine hydrate (10 equiv) in methanol at 50° C. to give tert-butyl (R)-(1-hydrazineyl-1,4-dioxo-4-(tritylamino)butan-2-yl)carbamate. This compound is treated with 3-phenylpropanal in ethanol at 70° C. for 12 hours to give intermediate tert-butyl (R,E)-(1,4-dioxo-1-(2-(3-phenylpropylidene)hydrazineyl)-4-(tritylamino)butan-2-yl)carbamate, which is further reacted with I₂ (1.2 equiv) and potassium carbonate (3 equiv) in DMSO to yield tert-butyl (R)-(3-oxo-1-(5-phenethyl-1,3,4-oxadiazol-2-yl)-3-(tritylamino)propyl)carbamate. This compound is deprotected with a solution of triethylsilane, dichloromethane, water, and trifluoroacetic acid, then reacted with octanoic anhydride and DIPEA in DMF. Upon completion, the reaction is worked up and purified by column chromatography to yield the title compound.

### Example 140: (R)-N-(3-amino-1-(5-ethyl-1,3,4-oxadiazol-2-yl)-3-oxopropyl)octanamide

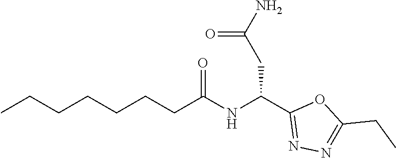 [SMILES: CCCCCCCC(=O)N[C@H](CC(N)=O)c1nnc(CC)o1]

The title compound may be synthesized according to the experimental procedure for Example 139, replacing the 3-phenylpropanal with propionaldehyde.

### Example 141: (R)-N-(3-amino-1-(5-(tert-butyl)-1,3,4-oxadiazol-2-yl)-3-oxopropyl)octanamide

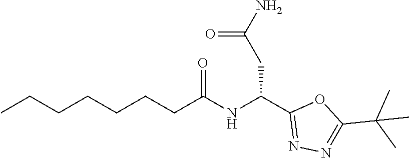 [SMILES: CCCCCCCC(=O)N[C@H](CC(N)=O)c1nnc(C(C)(C)C)o1]

The title compound may be synthesized according to the experimental procedure for Example 139, replacing the 3-phenylpropanal with pivalaldehyde.

### Example 142: (R)-3-(3-hexylureido)-3-(5-phenethyl-1,3,4-oxadiazol-2-yl)propenamide

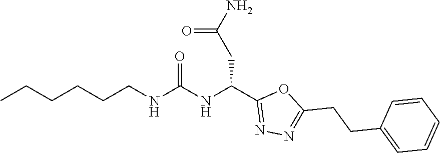 [SMILES: CCCCCCNC(=O)N[C@H](CC(N)=O)c1nnc(CCc2ccccc2)o1]

The title compound may be synthesized according to the experimental procedure for Example 139, replacing the octanoic anhydride with 1-isocyanatohexane.

### Example 143: hexyl (R)-(3-amino-3-oxo-1-(5-phenethyl-1,3,4-oxadiazol-2-yl)propyl)carbamate

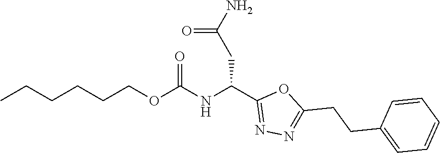 [SMILES: CCCCCCOC(=O)N[C@H](CC(N)=O)c1nnc(CCc2ccccc2)o1]

The title compound may be synthesized according to the experimental procedure for Example 139, replacing the octanoic anhydride with hexyl carbonochloridate.

### Example 144: (R)-N-(3-amino-3-oxo-1-(4-phenethyloxazol-2-yl)propyl)octanamide

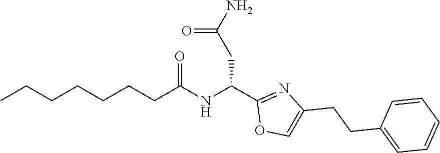 [SMILES: CCCCCCCC(=O)N[C@H](CC(N)=O)c1nc(CCc2ccccc2)co1]

N²-(tert-butoxycarbonyl)-N4-trityl-D-asparagine (1 equiv) is treated with HATU (1.05 equiv), DIPEA (2.1 equiv), and methyl 2-amino-4-phenylbutanoate (1.1 equiv) in DMF to give methyl 2-((R)-2-((tert-butoxycarbonyl)amino)-4-oxo-4-(tritylamino)butanamido)-4-phenylbutanoate. Methyl ester hydrolysis with LiOH yields 2-((R)-2-((tert-butoxycarbonyl)amino)-4-oxo-4-(tritylamino)butanamido)-4-phenylbutanoic acid which is then treated with HATU (1.05 equiv), HOAt (1.05 equiv), DIPEA (2.1 equiv), and N,O-dimethylhydroxylamine hydrochloride (1.1 equiv) in DMF followed by reduction with diisobutylaluminium hydride (1.05 equiv) in THF to yield tert-butyl ((2R)-1,4-dioxo-1-((1-oxo-4-phenylbutan-2-yl)amino)-4-(tritylamino)butan-2-yl)carbamate. This compound is reacted with triphenylphosphine (2 equiv), iodine (2 equiv), and triethylamine, followed by addition of saturated aqueous sodium pyrosulfite to the solution. The resulting product is deprotected with triethylsilane, water, dichloromethane, and TFA (10 equiv) to give (R)-3-amino-3-(4-phenethyloxazol-2-yl)propanamide and which is then reacted with octanoic anhydride (1.05 equiv) and N,N-diisopropylethylamine (1.1 equiv) in DMF. Purification by reverse phase C18 column chromatography and supercritical fluid chromatography yields the title compound.

### Example 145: (R)-N-(3-amino-1-(4-ethyloxazol-2-yl)-3-oxopropyl)octanamide

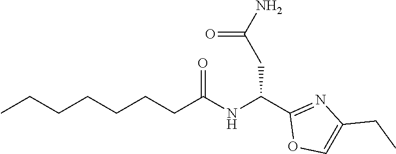 [SMILES: CCCCCCCC(=O)N[C@H](CC(N)=O)c1nc(CC)co1]

The title compound may be synthesized according to the experimental procedure for Example 144, replacing methyl 2-amino-4-phenylbutanoate with methyl 2-aminobutanoate.

### Example 146: (R)-N-(3-amino-1-(4-(tert-butyl)oxazol-2-yl)-3-oxopropyl)octanamide

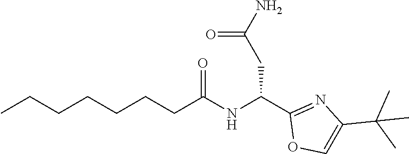 [SMILES: CCCCCCCC(=O)N[C@H](CC(N)=O)c1nc(C(C)(C)C)co1]

The title compound may be synthesized according to the experimental procedure for Example 144, replacing methyl 2-amino-4-phenylbutanoate with methyl 2-amino-3,3-dimethylbutanoate.

### Example 147: (R)-3-(3-hexylureido)-3-(4-phenethyloxazol-2-yl)propenamide

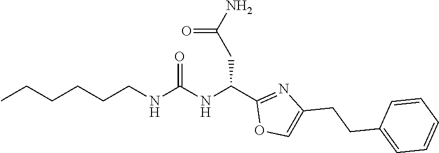 [SMILES: CCCCCCNC(=O)N[C@H](CC(N)=O)c1nc(CCc2ccccc2)co1]

The title compound may be synthesized according to the experimental procedure for Example 144, replacing the octanoic anhydride with 1-isocyanatohexane.

### Example 148: hexyl (R)-(3-amino-3-oxo-1-(4-phenethyloxazol-2-yl)propyl)carbamate

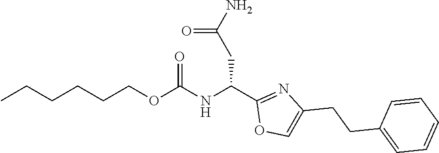 [SMILES: CCCCCCOC(=O)N[C@H](CC(N)=O)c1nc(CCc2ccccc2)co1]

The title compound may be synthesized according to the experimental procedure for Example 144, replacing the octanoic anhydride with hexyl carbonochloridate.

### Example 149: (R)-N-(3-amino-3-oxo-1-(3-phenethyl-1,2,4-oxadiazol-5-yl)propyl)octanamide

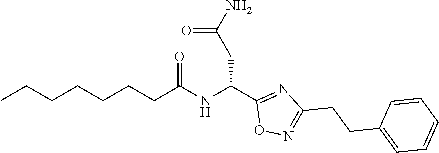 [SMILES: CCCCCCCC(=O)N[C@H](CC(N)=O)c1nc(CCc2ccccc2)no1]

Benzenepropanenitrile (1 equiv) is treated with potassium carbonate (2 equiv) and hydroxylamine hydrochloride to yield N-hydroxy-3-phenylpropanimidamide. Compound N-hydroxy-3-phenylpropanimidamide (1 equiv) is reacted with Nα-Boc-Nγ-trityl-D-asparagine (1.1 equiv), diisopropylcarbodiimide (1.1 equiv), and 1-hydroxybenzotriazole (1.1 equiv) to yield tert-butyl (R)-(1,4-dioxo-1-((3-phenylpropanimidamido)oxy)-4-(tritylamino)butan-2-yl)carbamate. This compound is stirred in acetic acid and acetonitrile at 90° C. for 12 hours. The resulting product is deprotected with triethylsilane, water, dichloromethane, and TFA (10 equiv), and stirred in octanoic anhydride (1.05 equiv) and N,N-diisopropylethylamine (1.1 equiv) in DMF. Upon completion, reaction is worked up and purified by reverse phase C18 column chromatography to yield the title compound.

### Example 150: (R)-N-(3-amino-1-(3-ethyl-1,2,4-oxadiazol-5-yl)-3-oxopropyl)octanamide

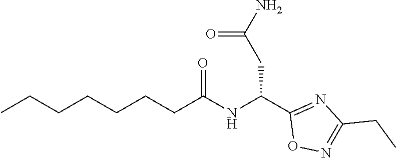 [SMILES: CCCCCCCC(=O)N[C@H](CC(N)=O)c1nc(CC)no1]

The title compound may be synthesized according to the experimental procedure for Example 149, replacing the benzenepropanenitrile with propiononitrile.

### Example 151: (R)-N-(3-amino-1-(3-(tert-butyl)-1,2,4-oxadiazol-5-yl)-3-oxopropyl)octanamide

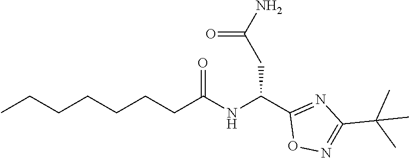 [SMILES: CCCCCCCC(=O)N[C@H](CC(N)=O)c1nc(C(C)(C)C)no1]

The title compound may be synthesized according to the experimental procedure for Example 149, replacing the benzenepropanenitrile with pivalonitrile.

### Example 152: (R)-3-(3-hexylureido)-3-(3-phenethyl-1,2,4-oxadiazol-5-yl)propenamide

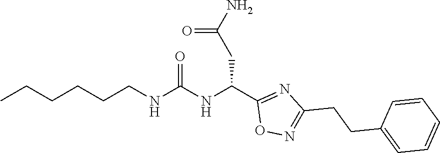 [SMILES: CCCCCCNC(=O)N[C@H](CC(N)=O)c1nc(CCc2ccccc2)no1]

The title compound may be synthesized according to the experimental procedure for Example 149, replacing the octanoic anhydride with 1-isocyanatohexane.

### Example 153: hexyl (R)-(3-amino-3-oxo-1-(3-phenethyl-1,2,4-oxadiazol-5-yl)propyl)carbamate

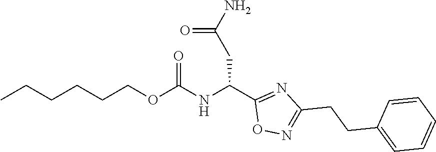 [SMILES: CCCCCCOC(=O)N[C@H](CC(N)=O)c1nc(CCc2ccccc2)no1]

The title compound may be synthesized according to the experimental procedure for Example 152, replacing the octanoic anhydride with hexyl carbonochloridate.

### Example 154: (R)-N-(3-amino-3-oxo-1-(1-phenethyl-1H-1,2,3-triazol-4-yl)propyl)octanamide

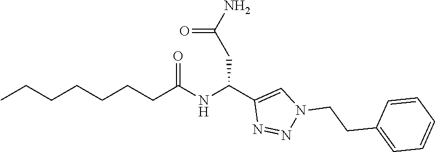 [SMILES: CCCCCCCC(=O)N[C@H](CC(N)=O)c1cn(CCc2ccccc2)nn1]

N²-(tert-butoxycarbonyl)-N⁴-trityl-D-asparagine is stirred with HOBt (1 equiv), DIC (1 equiv), and N,O-dimethylhydroxylamine hydrochloride 1 equiv) in DMF for 30 minutes, followed by the addition of DIPEA (1 equiv). Upon completion, the reaction is worked up and purified to yield tert-butyl (R)-(1-(methoxy(methyl)amino)-1,4-dioxo-4-(tritylamino)butan-2-yl)carbamate, which is then dissolved in DCM and reacted with diisobutylaluminum hydride (1.2 equiv) at −78° C. for 40 minutes. Excess hydride is quenched with anhydrous methanol and the resulting solution was warmed to 0° C. Potassium carbonate (2 equiv) and dimethyl (1-diazo-2-oxopropyl)phosphonate (1.2 equiv) is then added and the resulting solution is stirred. Upon completion, the reaction is worked up and purified to yield tert-butyl (R)-(5-oxo-5-(tritylamino)pent-1-yn-3-yl)carbamate, which is reacted with (2-azidoethyl)benzene in the presence of copper sulfate pentahydrate (0.01 equiv) and aqueous sodium ascorbate. Upon completion, the reaction is worked up and purified to give tert-butyl (R)-(3-oxo-1-(1-phenethyl-1H-1,2,3-triazol-4-yl)-3-(tritylamino)propyl)carbamate, which is then deprotected with a solution of triethylsilane, water, dichloromethane, and trifluoroacetic acid. The resulting compound (R)-(3-amino-3-oxo-1-(1-phenethyl-1H-1,2,3-triazol-4-yl)propyl)carbamic acid is then reacted with octanoic anhydride (1.5 equiv) and DIPEA (3 equiv), concentrated, and purified to yield (R)-N-(3-amino-3-oxo-1-(1-phenethyl-1H-1,2,3-triazol-4-yl)propyl)octanamide.

### Example 155: (R)-N-(3-amino-1-(1-ethyl-1H-1,2,3-triazol-4-yl)-3-oxopropyl)octanamide

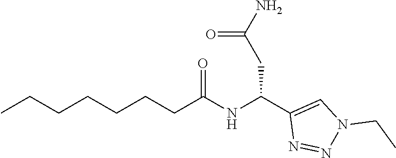 [SMILES: CCCCCCCC(=O)N[C@H](CC(N)=O)c1cn(CC)nn1]

The title compound may be synthesized according to the experimental procedure for Example 154, replacing (2-azidoethyl)benzene with azidoethane, which is prepared by treating ethyl bromide with sodium azide.

### Example 156: (R)-N-(3-amino-1-(1-(tert-butyl)-1H-1,2,3-triazol-4-yl)-3-oxopropyl)octanamide

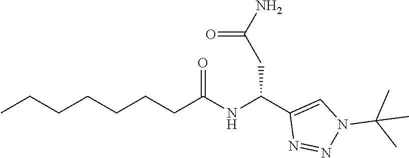 [SMILES: CCCCCCCC(=O)N[C@H](CC(N)=O)c1cn(C(C)(C)C)nn1]

The title compound may be synthesized according to the experimental procedure for Example 154, replacing (2-azidoethyl)benzene with 2-azido-2-methylpropane.

### Example 1: (R)-3-(3-hexylureido)-3-(1-phenethyl-1H-1,2,3-triazol-4-yl)propenamide

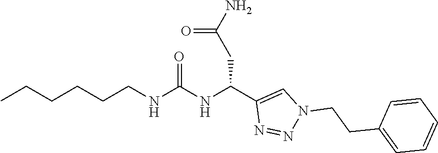 [SMILES: CCCCCCNC(=O)N[C@H](CC(N)=O)c1cn(CCc2ccccc2)nn1]

The title compound may be synthesized according to the experimental procedure for Example 154, replacing the octanoic anhydride with 1-isocyanatohexane.

### Example 161: hexyl (R)-(3-amino-3-oxo-1-(1-phenethyl-1H-1,2,3-triazol-4-yl)propyl)carbamate

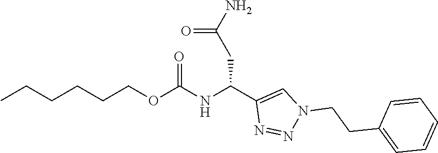 [SMILES: CCCCCCOC(=O)N[C@H](CC(N)=O)c1cn(CCc2ccccc2)nn1]

The title compound may be synthesized according to the experimental procedure for Example 154, replacing the octanoic anhydride with hexyl carbonochloridate.

### Example 162: (R)-N-(4-amino-1-hydrazineyl-1,4-dioxobutan-2-yl)octanamide

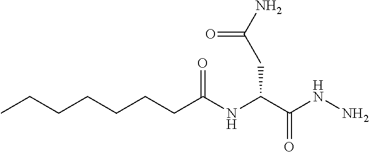 [SMILES: CCCCCCCC(=O)N[C@H](CC(N)=O)C(=O)NN]

Methyl N⁴-trityl-D-asparaginate is treated with octanoic anhydride (1.5 equiv) and triethylamine (3 equiv). Upon completion, the reaction is worked up and purified to yield methyl N²-octanoyl-N⁴-trityl-D-asparaginate, which is then reacted with hydrazine hydrate to yield tert-butyl (R)-(1-hydrazineyl-1,4-dioxo-4-(tritylamino)butan-2-yl)carbamate.

### Example 163: (R)-3-(3-hexylureido)-4-hydrazineyl-4-oxobutanamide

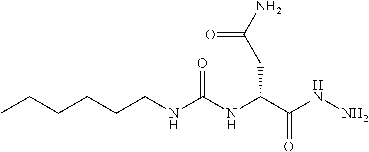 [SMILES: CCCCCCNC(=O)N[C@H](CC(N)=O)C(=O)NN]

The title compound may be synthesized according to the experimental procedure for Example 162, replacing octanoic anhydride with 1-isocyanatohexane.

### Example 164: hexyl (R)-(4-amino-1-hydrazineyl-1,4-dioxobutan-2-yl)carbamate

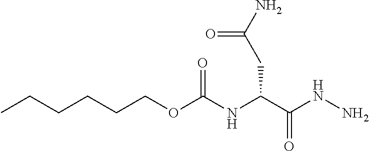 [SMILES: CCCCCCOC(=O)N[C@H](CC(N)=O)C(=O)NN]

The title compound may be synthesized according to the experimental procedure for Example 162, replacing the octanoic anhydride with hexyl carbonochloridate.

### Example 165: (R)-N-(3-amino-1-cyano-3-oxopropyl)octanamide

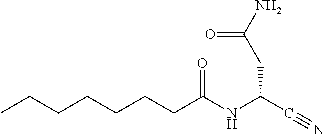 [SMILES: CCCCCCCC(=O)N[C@@H](C#N)CC(N)=O]

To a solution of N²-(tert-butoxycarbonyl)-N⁴-trityl-D-asparagine and 1-hydroxybenzotriazole (1.1 equiv) in DCM at 0° C., EDCI (1.1 equiv) is added. Reaction is warmed to room temperature for 30 minutes, then chilled to 0° C. and ammonia (4 equiv) is added. Reaction is stirred at room temperature for 30 minutes, concentrated, and purified by column chromatography. The resulting tert-butyl (R)-(1-amino-1,4-dioxo-4-(tritylamino)butan-2-yl)carbamate is treated with T3P and DIPEA in THF to give tert-butyl (R)-(1-cyano-3-oxo-3-(tritylamino)propyl)carbamate, which is then deprotected with HCl in ethyl acetate to yield (R)-3-amino-3-cyanopropanamide. (R)-3-amino-3-cyanopropanamide is then treated with octanoic anhydride (1.5 equiv) and triethylamine (3 equiv). Upon completion, the reaction is worked up and purified to yield (R)-N-(3-amino-1-cyano-3-oxopropyl)octanamide.

### Example 166: (R)-3-cyano-3-(3-hexylureido)propenamide

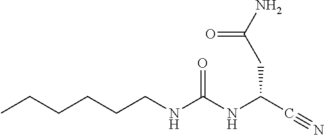 [SMILES: CCCCCCNC(=O)N[C@@H](C#N)CC(N)=O]

To a solution of N²-(tert-butoxycarbonyl)-N⁴-trityl-D-asparagine and 1-hydroxybenzotriazole (1.1 equiv) in DCM at 0° C., EDCI (1.1 equiv) is added. Reaction is warmed to room temperature for 30 minutes, then chilled to 0° C. and ammonia (4 equiv) is added. Reaction is stirred at room temperature for 30 minutes, concentrated, and purified by column chromatography. The resulting tert-butyl (R)-(1-amino-1,4-dioxo-4-(tritylamino)butan-2-yl)carbamate is treated with T3P and DIPEA in THF to give tert-butyl (R)-(1-cyano-3-oxo-3-(tritylamino)propyl)carbamate, which is then deprotected with HCl in ethyl acetate to yield (R)-3-amino-3-cyanopropanamide. (R)-3-amino-3-cyanopropanamide is then treated with 1-isocyanatohexane (1.1 equiv) and triethylamine (1.5 equiv). Upon completion, reaction as worked up and purified by column chromatography to yield (R)-3-cyano-3-(3-hexylureido)propenamide.

### Example 167: hexyl (R)-(3-amino-1-cyano-3-oxopropyl)carbamate

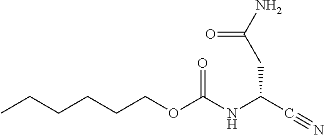 [SMILES: CCCCCCOC(=O)N[C@@H](C#N)CC(N)=O]

To a solution of N²-(tert-butoxycarbonyl)-N⁴-trityl-D-asparagine and 1-hydroxybenzotriazole (1.1 equiv) in DCM at 0° C., EDCI (1.1 equiv) is added. Reaction is warmed to room temperature for 30 minutes, then chilled to 0° C. and ammonia (4 equiv) is added. Reaction is stirred at room temperature for 30 minutes, concentrated, and purified by column chromatography. The resulting tert-butyl (R)-(1-amino-1,4-dioxo-4-(tritylamino)butan-2-yl)carbamate is treated with T3P and DIPEA in THF to give tert-butyl (R)-(1-cyano-3-oxo-3-(tritylamino)propyl)carbamate, which is then deprotected with HCl in ethyl acetate to yield (R)-3-amino-3-cyanopropanamide. (R)-3-amino-3-cyanopropanamide is then treated with hexyl carbonochloridate (1.1 equiv) and triethylamine (1.1 equiv). Upon completion, reaction is worked up and purified by column chromatography to yield hexyl (R)-(3-amino-1-cyano-3-oxopropyl)carbamate.

### Example 168: (R)-N′-(4-chlorophenethyl)-2-octanamidosuccinamide

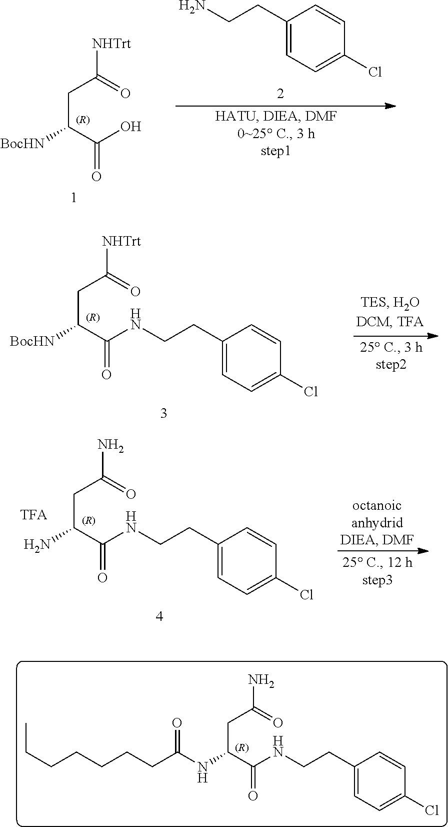

### Step 1

To a solution of (2R)-2-(tert-butoxycarbonylamino)-4-oxo-4-(tritylamino)butanoic acid (2 g, 4.21 mmol, 1 eq) and 2-(4-chlorophenyl)ethanamine (787.06 mg, 5.06 mmol, 709.07 uL, 1.2 eq) in DMF (10 mL) was added HATU (1.92 g, 5.06 mmol, 1.2 eq) and DIEA (1.20 g, 9.27 mmol, 1.61 mL, 2.2 eq) at 0° C. The mixture was stirred at 25° C. for 3 hr. TLC indicated (2R)-2-(tert-butoxycarbonylamino)-4-oxo-4-(tritylamino)butanoic acid was consumed completely and one new spot formed. The reaction mixture was quenched by addition H₂O 60 mL at 0° C., and the precipitate was filtered and filter cake was concentrated under reduced pressure to give compound tert-butyl N-[(1R)-1-[2-(4-chlorophenyl)ethylcarbamoyl]-3-oxo-3-(tritylamino)propyl]carbamate (2.2 g, 2.05 mmol, 48.61% yield, 57% purity) as a white solid.

### Step 2

A mixture of tert-butyl N-[(1R)-1-[2-(4-chlorophenyl)ethylcarbamoyl]-3-oxo-3-(tritylamino) propyl]carbamate (800 mg, 1.31 mmol, 1 eq), TES (748.99 mg, 3.27 mmol, 0.5 mL, 2.5 eq), H₂O (500.00 mg, 27.75 mmol, 0.5 mL, 21.23 eq), DCM (1.32 g, 15.54 mmol, 1 mL, 11.89 eq) and TFA (27.72 g, 243.12 mmol, 18 mL, 186.03 eq) was stirred at 25° C. for 3 hr under N₂ atmosphere. TLC indicated tert-butyl N-[(1R)-1-[2-(4-chlorophenyl)ethylcarbamoyl]-3-oxo-3-(tritylamino) propyl]carbamate was consumed completely and one new spot formed. The reaction mixture was concentrated under reduced pressure to give compound (2R)-2-amino-N-[2-(4-chlorophenyl)ethyl]butanediamide (450 mg, crude, TFA) as a white solid.

### Step 3

A mixture of (2R)-2-amino-N-[2-(4-chlorophenyl)ethyl]butanediamide (450 mg, 1.17 mmol, 1 eq, TFA) in DMF (5 mL) was added DIEA (454.66 mg, 3.52 mmol, 612.74 uL, 3 eq), octanoyl octanoate (475.63 mg, 1.76 mmol, 1.5 eq). Then the mixture was stirred at 25° C. for 12 hr under N₂ atmosphere. The mixture was filtered and the filter cake was concentrated under reduced pressure to give compound (2R)-N-[2-(4-chlorophenyl)ethyl]-2-(octanoylamino)butanediamide (200 mg, 500.10 umol, 42.65% yield, 99% purity) as a white solid.

### Example 169: (R)-2-octanamido-N′-(4-(trifluoromethyl)phenethyl)succinamide

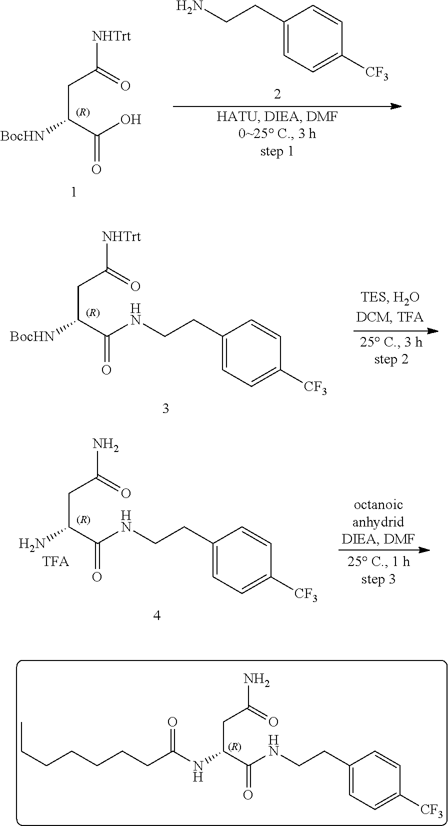

### Step 1

To a solution of (2R)-2-(tert-butoxycarbonylamino)-4-oxo-4-(tritylamino)butanoic acid (1 g, 2.11 mmol, 1 eq) and 2-[4-(trifluoromethyl)phenyl]ethanamine (478.38 mg, 2.53 mmol, 1.2 eq) in DMF (15 mL) was added HATU (961.50 mg, 2.53 mmol, 1.2 eq) and DIEA (599.17 mg, 4.64 mmol, 807.51 uL, 2.2 eq) at 0° C. Then the mixture was stirred at 25° C. for 3 hr. The reaction mixture was quenched by addition H₂O 30 mL at 0° C., and then filtered and the filter cake was concentrated under reduced pressure to give a residue. The crude product was triturated with Petroleum ether/Ethyl acetate=5/1 (15 mL) at 25° C. for 30 min to give tert-butyl N-[(1R)-3-oxo-1-[2-[4-(trifluoromethyl)phenyl]ethylcarbamoyl]-3-(tritylamino)propyl]carbamate (1 g, 1.55 mmol, 73.49% yield) as a white solid.

### Step 2

A mixture of tert-butyl N-[(1R)-3-oxo-1-[2-[4-(trifluoromethyl)phenyl]ethylcarbamoyl]-3-(tritylamino)propyl]carbamate (0.9 g, 1.39 mmol, 1 eq) in TES (0.5 mL), H₂O (0.5 mL), DCM (1 mL) and TFA (18 mL) was degassed and purged with N₂ for 3 times. And then the mixture was stirred at 25° C. for 3 hr under N₂ atmosphere. LCMS showed desired compound was detected. The reaction mixture was concentrated under reduced pressure to give (2R)-2-amino-N-[2-[4-(trifluoromethyl)phenyl]ethyl]butanediamide (600 mg, crude, TFA) as a yellow solid.

### Step 3

To a solution of (2R)-2-amino-N-[2-[4-(trifluoromethyl)phenyl]ethyl]butanediamide (600 mg, 1.44 mmol, 1 eq, TFA) in DMF (10 mL) was added octanoyl octanoate (583.19 mg, 2.16 mmol, 1.5 eq) and DIEA (557.48 mg, 4.31 mmol, 751.32 uL, 3 eq). The mixture was stirred at 25° C. for 1 hr. The reaction mixture was concentrated under reduced pressure to give a residue. The residue was purified by prep-HPLC (column: Waters Xbridge Prep OBD C18 150*40 mm*10 um; mobile phase: [water(10 mM NH₄HCO₃)-ACN]; B %: 30%-60%, 8 min) to give (2R)-2-(octanoylamino)-N-[2-[4-(trifluoromethyl)phenyl]ethyl]butanediamide (207 mg, 472.34 umol, 32.85% yield, 98% purity) as a white solid.

### Example 170: (R)-2-octanamido-N′-(4-(trifluoromethyl)phenethyl)succinamide

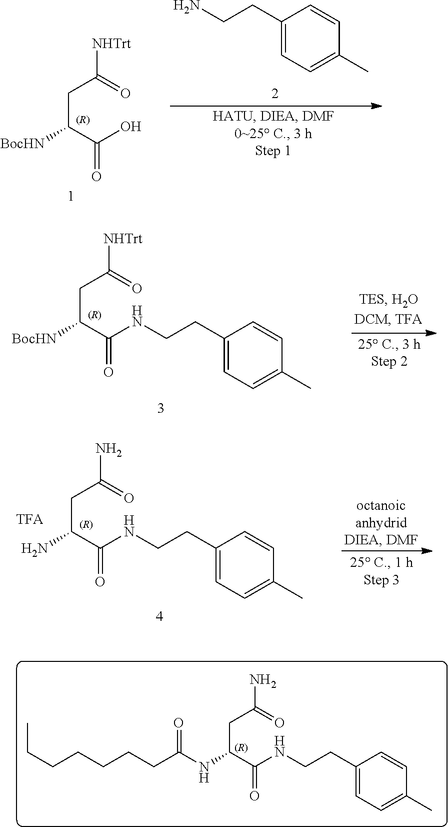

### Step 1

To a solution of (2R)-2-(tert-butoxycarbonylamino)-4-oxo-4-(tritylamino)butanoic acid (1 g, 2.11 mmol, 1 eq) and 2-(p-tolyl)ethanamine (341.90 mg, 2.53 mmol, 367.63 uL, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 3 h. LC-MS showed the desired compound was detected. The reaction mixture was poured to water (100 mL) and then the mixture was filtered and the filter cake was concentrated under reduced pressure to give compound tert-butyl N-[(1R)-3-oxo-1-[2-(p-tolyl)ethylcarbamoyl]-3-(tritylamino)propyl]carbamate (1.00 g, 1.59 mmol, 75.38% yield, 94% purity) as white solid.

### Step 2

A mixture of triethylsilane (364.00 mg, 3.13 mmol, 0.5 mL, 2.06 eq), H₂O (500.00 mg, 27.75 mmol, 0.5 mL, 18.25 eq), DCM (1.32 g, 15.54 mmol, 1 mL, 10.22 eq) and TFA (13.09 g, 129.32 mmol, 18 mL, 85.03 eq) was added tert-butyl N-[(1R)-3-oxo-1-[2-(p-tolyl)ethylcarbamoyl]-3-(tritylamino)propyl]carbamate (900 mg, 1.52 mmol, 1 eq) and then the mixture was stirred at 25° C. for 3 h under N2 atmosphere. LC-MS showed the desired compound was detected. The reaction mixture was concentrated under reduced pressure to give the crude product (2R)-2-amino-N-[2-(p-tolyl)ethyl]butanediamide (550 mg, crude, TFA) as white solid. It was used into the next step without further purification.

### Step 3

To a solution of (2R)-2-amino-N-[2-(p-tolyl)ethyl]butanediamide (550 mg, 1.51 mmol, 1 eq, TFA) in DMF (10 mL) was added DIEA (586.93 mg, 4.54 mmol, 791.01 uL, 3 eq) and octanoyl octanoate (614.00 mg, 2.27 mmol, 1.5 eq). The mixture was stirred at 25° C. for 1 h. LC-MS showed the desired compound was detected. The reaction mixture was concentrated under reduced pressure to give a residue. The residue was purified by prep-HPLC ([water(10 mM NH₄HCO₃)-ACN]) to give compound (2R)-2-(octanoylamino)-N-[2-(p-tolyl)ethyl]butanediamide (110 mg, 290.01 umol, 15.34% yield, 99% purity) as white solid.

### Example 171: methyl (R)-4-(2-(4-amino-2-octanamido-4-oxobutanamido)ethyl)benzoate and (R)-4-(2-(4-amino-2-octanamido-4-oxobutanamido)ethyl)benzoic acid

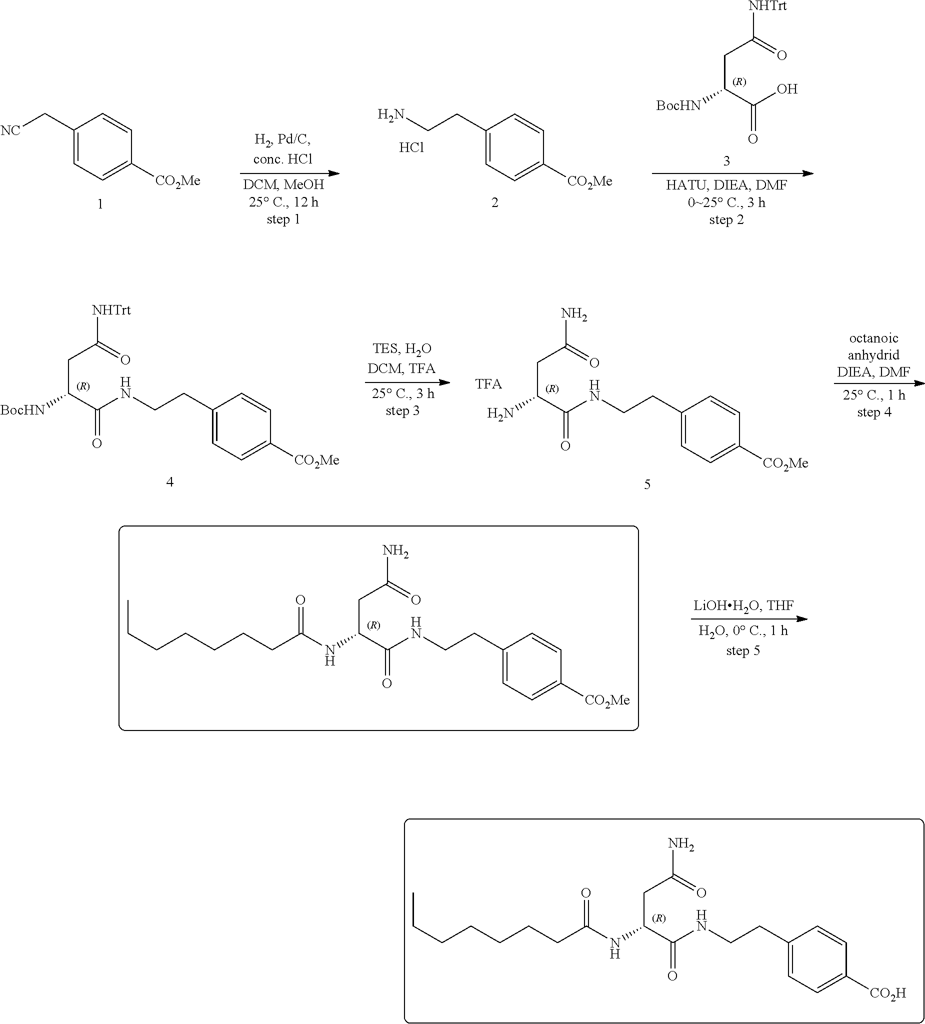

### Step 1

A mixture of methyl 4-(cyanomethyl)benzoate (2 g, 11.42 mmol, 1 eq) and Pd/C (1 g, 10% purity) in DCM (40 mL), MeOH (40 mL) and conc. HCl (3 mL) was degassed and purged with H₂ for 3 times, and then the mixture was stirred at 25° C. for 12 hr under H₂ (50 Psi) atmosphere. The reaction mixture was filtered and the filtrate was concentrated under reduced pressure to give a residue. The residue was triturated with DCM (10 mL) at 25° C. for 30 min to give methyl 4-(2-aminoethyl)benzoate (1.6 g, 7.42 mmol, 64.98% yield, HCl) as a light yellow solid.

### Step 2

To a solution of (2R)-2-(tert-butoxycarbonylamino)-4-oxo-4-(tritylamino)butanoic acid (2 g, 4.21 mmol, 1 eq) and methyl 4-(2-aminoethyl)benzoate (1.09 g, 5.06 mmol, 1.2 eq, HCl) in DMF (30 mL) was added HATU (1.92 g, 5.06 mmol, 1.2 eq) and DIEA (1.20 g, 9.27 mmol, 1.62 mL, 2.2 eq) at 0° C. The mixture was stirred at 25° C. for 3 hr. The reaction mixture was quenched by addition H₂O 50 mL at 0° C., and then filtered and the filter cake was concentrated under reduced pressure to give methyl 4-[2-[[(2R)-2-(tert-butoxycarbonylamino)-4-oxo-4-(tritylamino)butanoyl]amino]ethyl]benzoate (2.5 g, 3.93 mmol, 93.31% yield) as a yellow solid.

### Step 3

A mixture of methyl 4-[2-[[(2R)-2-(tert-butoxycarbonylamino)-4-oxo-4-(tritylamino)butanoyl]amino]ethyl]benzoate (2 g, 3.15 mmol, 1 eq), TES (0.5 mL), H₂O (0.5 mL), DCM (1 mL) and TFA (18 mL) was stirred at 25° C. for 3 hr under N2 atmosphere. The reaction mixture was concentrated under reduced pressure to give methyl 4-[2-[[(2R)-2,4-diamino-4-oxo-butanoyl]amino]ethyl]benzoate (1.3 g, crude, TFA) as a yellow solid.

### Step 4

To a solution of methyl 4-[2-[[(2R)-2,4-diamino-4-oxo-butanoyl]amino]ethyl]benzoate (1.3 g, 3.19 mmol, 1 eq, TFA) in DMF (5 mL) was added octanoyl octanoate (1.29 g, 4.79 mmol, 1.5 eq) and DIEA (1.24 g, 9.57 mmol, 1.67 mL, 3 eq). The mixture was stirred at 25° C. for 1 hr. The reaction mixture was filtered and the filter cake was concentrated under reduced pressure to give methyl 4-[2-[[(2R)-4-amino-2-(octanoylamino)-4-oxo-butanoyl]amino]ethyl]benzoate (1.3 g, crude) as a white solid.

### Step 5

To a mixture of methyl 4-[2-[[(2R)-4-amino-2-(octanoylamino)-4-oxo-butanoyl]amino]ethyl]benzoate (200 mg, 476.74 umol, 1 eq) in THF (2 mL) and H₂O (2 mL) was added LiOH·H₂O (20.01 mg, 476.74 umol, 1 eq) in one portion at 0° C. The mixture was stirred at 0° C. for 1 hr. The reaction mixture was slowly poured in to the H₂O (10 ml) and extracted with ethyl acetate (5 mL*3). The combined organic layers were filtered and concentrated under reduced pressure to give a residue. The mixture was purified by p-HPLC(column: Waters Xbridge Prep OBD C18 150*40 mm*10 um; mobile phase: [water(0.05% NH₃H₂O+10 mM NH₄HCO₃)-ACN]; B %: 5%-30%, 8 min) to give 4-[2-[[(2R)-4-amino-2-(octanoylamino)-4-oxo-butanoyl]amino]ethyl]benzoic acid (6 mg, 16.18 umol, 3.39% yield, 81% purity) as a white solid.

### Example 172: (R)-N′-(4-methoxyphenethyl)-2-octanamidosuccinamide and (R)-N′-(4-hydroxyphenethyl)-2-octanamidosuccinamide

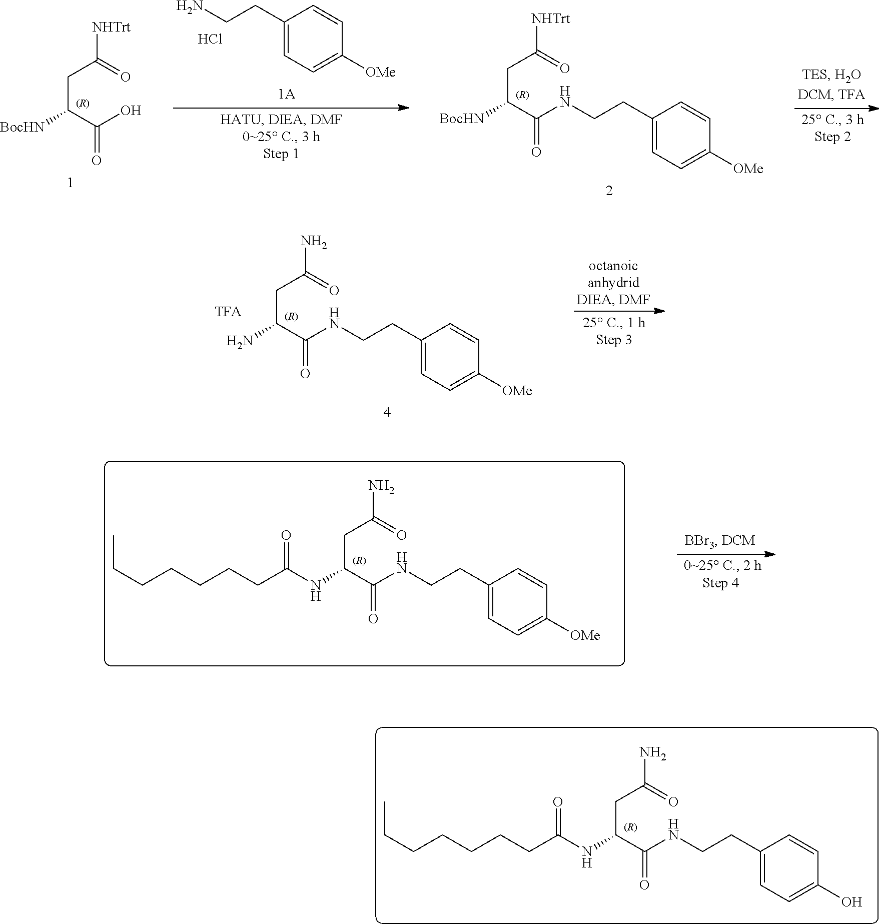

### Step 1

To a solution of (2R)-2-(tert-butoxycarbonylamino)-4-oxo-4-(tritylamino)butanoic acid (1 g, 2.11 mmol, 1 eq) and 2-(4-methoxyphenyl)ethanamine (382.36 mg, 2.53 mmol, 371.22 uL, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 3 h. LC-MS showed the desired compound was detected. The reaction mixture was poured to water (100 mL) and then the mixture was filtered and the filter cake was concentrated under reduced pressure to give compound tert-butyl N-[(1R)-1-[2-(4-methoxyphenyl)ethylcarbamoyl]-3-oxo-3-(tritylamino) propyl]carbamate (1.1 g, 1.81 mmol, 85.89% yield) as white solid.

### Step 2

A mixture of triethylsilane (364.00 mg, 3.13 mmol, 0.5 mL, 1.73 eq), H₂O (500.00 mg, 27.75 mmol, 0.5 mL, 15.33 eq), DCM (1.32 g, 15.54 mmol, 1 mL, 8.59 eq) and TFA (27.72 g, 243.11 mmol, 18 mL, 134.31 eq) was added tert-butyl N-[(1R)-1-[2-(4-methoxyphenyl)ethylcarbamoyl]-3-oxo-3-(tritylamino)propyl]carbamate (1.1 g, 1.81 mmol, 1 eq). The mixture was stirred at 25° C. for 3 h under N₂ atmosphere. LC-MS showed the desired compound was detected. The reaction mixture was concentrated under reduced pressure to give the product (2R)-2-amino-N-[2-(4-methoxyphenyl)ethyl]butanediamide (480 mg, crude, TFA) as white solid.

### Step 3

To a solution of (2R)-2-amino-N-[2-(4-methoxyphenyl)ethyl]butanediamide (480 mg, 1.27 mmol, 1 eq, TFA) in DMF (10 mL) was added DIPEA (490.63 mg, 3.80 mmol, 661.22 uL, 3 eq) and octanoyl octanoate (410.61 mg, 1.52 mmol, 1.2 eq). The mixture was stirred at 25° C. for 1 h. LC-MS showed the desired compound was detected. The reaction mixture was concentrated under reduced pressure to give a residue. The residue was purified by prep-HPLC ([water(10 mM NH₄HCO₃)-ACN]) to give compound (2R)-N-[2-(4-methoxyphenyl)ethyl]-2-(octanoylamino) butanediamide (52 mg, 132.82 umol, 10.50% yield) as white solid.

### Step 4

To a solution of (2R)-N-[2-(4-methoxyphenyl)ethyl]-2-(octanoylamino)butanediamide (25 mg, 63.86 umol, 1 eq) in DCM (3 mL) was added BBr₃ (79.99 mg, 319.28 umol, 30.76 uL, 5 eq) at 0° C. The mixture was stirred at 25° C. for 2 h. LC-MS showed the desired compound was detected. The reaction mixture was concentrated under reduced pressure to give a residue. The residue was purified by prep-HPLC([water(10 mM NH₄HCO₃)-ACN]) to give compound (2R)-N-[2-(4-hydroxyphenyl)ethyl]-2-(octanoylamino)butanediamide (7 mg, 17.62 umol, 11.08% yield, 95% purity) as white solid.

### Example 173: (R)-N′-benzyl-2-octanamidosuccinamide

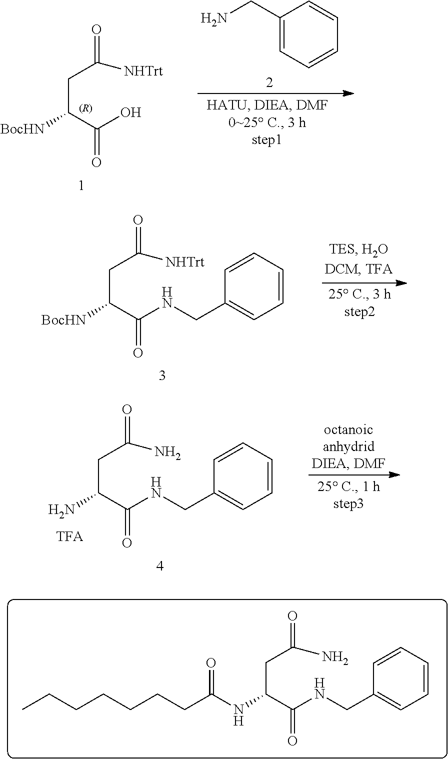

### Step 1

To a solution of (2R)-2-(tert-butoxycarbonylamino)-4-oxo-4-(tritylamino)butanoic acid (1 g, 2.11 mmol, 1 eq) and phenylmethanamine (270.96 mg, 2.53 mmol, 275.65 uL, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 3 hr. The reaction mixture was quenched by addition H₂O 60 mL at 0° C., and the precipitate was filtered and filter cake was concentrated. Compound tert-butyl N-[(1R)-1-(benzylcarbamoyl)-3-oxo-3-(tritylamino)propyl]carbamate (1 g, 1.61 mmol, 76.61% yield, 91% purity) was obtained as a white solid without purification.

### Step 2

A mixture of tert-butyl N-[(1R)-1-(benzylcarbamoyl)-3-oxo-3-(tritylamino)propyl]carbamate (0.8 g, 1.42 mmol, 1 eq), TES (0.5 mL), H₂O (0.5 mL), DCM (1 mL), TFA (18 mL) was stirred at 25° C. for 3 hr under N₂ atmosphere. TLC indicated tert-butyl N-[(1R)-1-(benzylcarbamoyl)-3-oxo-3-(tritylamino)propyl]carbamate was consumed completely and one new spot formed. The reaction mixture was concentrated under reduced pressure to give compound (2R)-2-amino-N-benzyl-butanediamide (0.4 g, crude, TFA) as a white solid.

### Step 3

A mixture of (2R)-2-amino-N-benzyl-butanediamide (0.4 g, 1.19 mmol, 1 eq, TFA) in DMF (5 mL) was added DIEA (462.58 mg, 3.58 mmol, 623.42 uL, 3 eq), octanoyl octanoate (483.91 mg, 1.79 mmol, 1.5 eq) and was degassed and purged with N₂ for 3 times. The mixture was stirred at 25° C. for 1 hr under N2 atmosphere. The reaction mixture was diluted with DMF 20 mL. The residue was purified by prep-HPLC (column: Phenomenex luna C18 100*40 mm*5 um; mobile phase: [water(0.1% TFA)-ACN]; B %: 10%-53%, 8 min) to give compound (2R)-N-benzyl-2-(octanoylamino)butanediamide (88 mg, 245.68 umol, 20.59% yield, 97% purity) as a white solid.

### Example 174: (R)-N′-(2-cyclohexylethyl)-2-octanamidosuccinamide

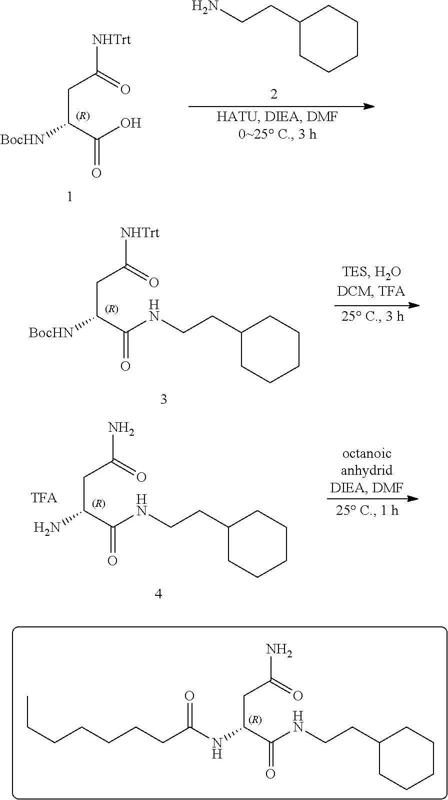

### Step 1

To a solution of (2R)-2-(tert-butoxycarbonylamino)-4-oxo-4-(tritylamino)butanoic acid (1.5 g, 3.16 mmol, 1 eq) and 2-cyclohexylethanamine hydrochloride (620.88 mg, 3.79 mmol, 1.2 eq) in DMF (15 mL) was added HATU (1.44 g, 3.79 mmol, 1.2 eq) and DIEA (898.75 mg, 6.95 mmol, 1.21 mL, 2.2 eq) at 0° C. The mixture was stirred at 25° C. for 3 hr. The reaction mixture was quenched by addition H₂O 30 mL at 0° C., and then filtered and the filter cake was concentrated under reduced pressure to give N-[(1R)-1-(2-cyclohexylethylcarbamoyl)-3-oxo-3-(tritylamino) propyl]carbamate (1.5 g, 2.57 mmol, 81.29% yield) as a white solid.

### Step 2

A mixture of tert-butyl N-[(1R)-1-(2-cyclohexylethylcarbamoyl)-3-oxo-3-(tritylamino)propyl]carbamate (0.8 g, 1.37 mmol, 1 eq), TES (0.5 mL), H₂O (0.5 mL), DCM (1 mL) and TFA (18 mL) was stirred at 25° C. for 3 hr under N₂ atmosphere. The reaction mixture was concentrated under reduced pressure to give (2R)-2-amino-N-(2-cyclohexylethyl)butanediamide (500 mg, crude, TFA) as a yellow solid.

### Step 3

To a solution of (2R)-2-amino-N-(2-cyclohexylethyl)butanediamide (0.5 g, 1.41 mmol, 1 eq, TFA) in DMF (5 mL) was added octanoyl octanoate (570.72 mg, 2.11 mmol, 1.5 eq) and DIEA (545.54 mg, 4.22 mmol, 735.23 uL, 3 eq). The mixture was stirred at 25° C. for 1 hr. The reaction mixture was concentrated under reduced pressure to give a residue. The residue was purified by prep-HPLC (column: Waters Xbridge Prep OBD C18 150*40 mm*10 um; mobile phase: [water(10 mM NH₄HCO₃)-ACN]; B %: 40%-70%, 8 min), prep-TLC (SiO₂, DCM: MeOH=10:1) and triturated with PE:EA=5:1 (0.5 mL) at 25° C. for 30 min to give (2R)-N-(2-cyclohexylethyl)-2-(octanoylamino)butanediamide (33 mg, 87.10 umol, 6.19% yield, 97% purity) as a white solid.

### Example 175: (R)-2-octanamido-N′-((R)-1-phenylpropan-2-yl)succinimide and (R)-2-octanamido-N′-((S)-1-phenylpropan-2-yl)succinimide

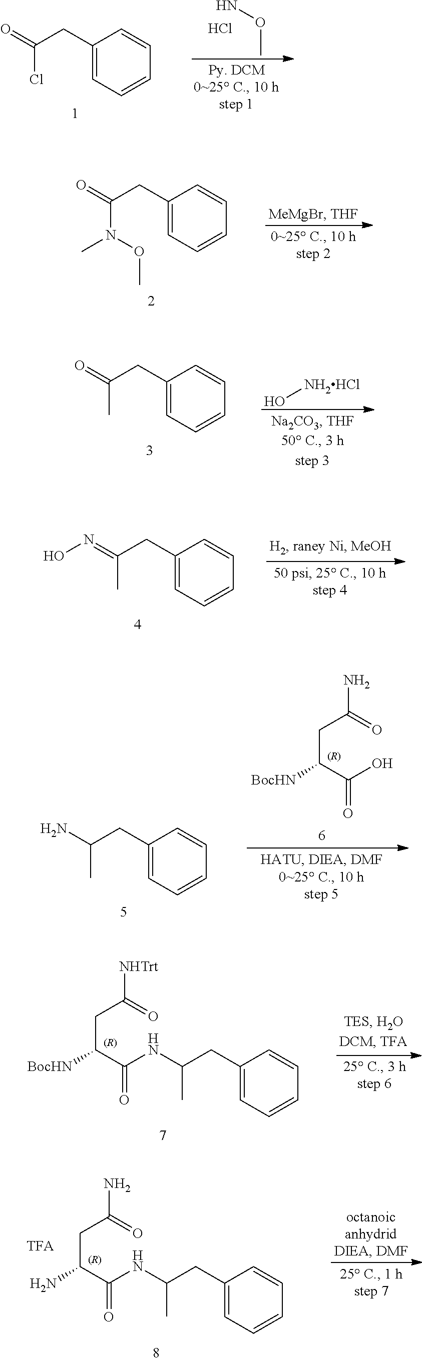

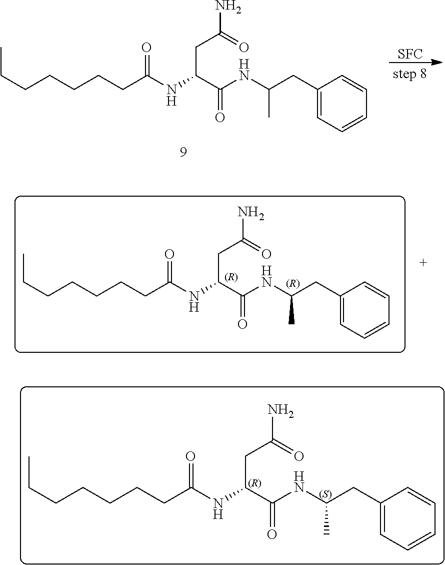

### Step 1

To a solution of N-methoxymethanamine hydrochloride (9.46 g, 97.03 mmol, 1 eq) and pyridine (16.88 g, 213.46 mmol, 17.23 mL, 2.2 eq) in DCM (500 mL) was dropwise added 2-phenylacetyl chloride (15 g, 97.03 mmol, 12.93 mL, 1 eq) at 0° C. The mixture was stirred at 25° C. for 10 hr under N₂ atmosphere. The reaction mixture was quenched by addition 0.1 NH₄Cl 100 mL at 0° C., and extracted with EtOAc 90 mL (30 mL*3). The combined organic layers were washed with brine 100 mL, dried over Na₂SO₄, filtered and concentrated under reduced pressure to give compound N-methoxy-N-methyl-2-phenyl-acetamide (10 g, 55.80 mmol, 57.51% yield) as yellow oil.

¹H NMR (400 MHz, CDCl₃)

### Step 2

To a mixture of N-methoxy-N-methyl-2-phenyl-acetamide (9.2 g, 51.33 mmol, 1 eq) in THF (200 mL) was added MeMgBr (3 M, 25.67 mL, 1.5 eq) at 0° C., then the mixture was stirred at 25° C. for 10 hr under N₂ atmosphere. The reaction mixture was quenched by addition 0.1 N HCl 200 mL at 0° C. and extracted with EtOAc 900 mL (300 mL*3). The combined organic layers were washed with brine 250 mL, dried over Na₂SO₄, filtered and the filtrate was concentrated under reduced pressure to give a compound 1-phenylpropan-2-one (5 g, 37.26 mmol, 72.59% yield) as yellow oil.

### Step 3

The mixture of 1-phenylpropan-2-one (2.00 g, 14.91 mmol, 1 eq) and hydroxylamine hydrochloride (2.07 g, 29.83 mmol, 2 eq) and Na₂CO₃ (3.16 g, 29.83 mmol, 2 eq) in a solution of THF (60 mL) and H₂O (30 mL) was stirred at 50° C. for 10 hr under N2 atmosphere. The reaction mixture was diluted with H₂O 100 mL and extracted with EtOAc 300 mL (100 mL*3). The combined organic layers were washed with brine 50 mL, dried over Na₂SO₄, filtered and concentrated under reduced pressure to give compound 1-phenylpropan-2-one oxime (1.5 g, crude) as yellow oil.

### Step 4

The mixture of 1-phenylpropan-2-one oxime (3 g, 20.11 mmol, 1 eq) and Raney nickel (1.72 g, 20.11 mmol, 1 eq) in MeOH (100 mL) was stirred at 25° C. for 10 hr under H₂ atmosphere at 50 psi. The reaction mixture was filtered and the filtrate was concentrated under reduced pressure to give a residue. The residue was purified by column chromatography (SiO₂, DCM: MeOH=5:1) to give compound 1-phenylpropan-2-amine (1.7 g, 12.57 mmol, 62.53% yield) as yellow oil.

### Step 5

The mixture of (2R)-2-(tert-butoxycarbonylamino)-4-oxo-4-(tritylamino)butanoic acid (2.5 g, 5.27 mmol, 1 eq) and 1-phenylpropan-2-amine (783.52 mg, 5.80 mmol, 1.1 eq) in DMF (5 mL) was added HATU (2.20 g, 5.80 mmol, 1.1 eq) and DIEA (1.36 g, 10.54 mmol, 1.84 mL, 2 eq) at 0° C. and the mixture was stirred at 25° C. for 10 hr under N₂ atmosphere. The reaction mixture was quenched by addition H₂O 100 mL at 0° C., filtered and the filter cake was concentrated under reduced pressure to give a residue. The residue was washed with EtOAc (5 ml), filtered and the filter cake was concentrated under reduced pressure to give compound tert-butyl N-[(1R)-1-[(1-methyl-2-phenyl-ethyl)carbamoyl]-3-oxo-3-(tritylamino)propyl]carbamate (3 g, 3.95 mmol, 75.06% yield, 78% purity) as a white solid.

### Step 6

A solution of tert-butyl N-[(1R)-1-[(1-methyl-2-phenyl-ethyl)carbamoyl]-3-oxo-3-(tritylamino)propyl]carbamate (800 mg, 1.35 mmol, 1 eq), TES (0.25 mL), H₂O (0.25 mL), DCM (0.25 mL) and TFA (9 mL) was stirred at 25° C. for 3 hr. The reaction mixture was filtered and filter cake discarded and filtrate concentrated under reduced pressure to give a residue. The crude product was triturated with EtOAc=(50 mL) at 25° C. for 20 min and filter cake concentrated under reduced pressure to give compound (2R)-2-amino-N-(1-methyl-2-phenyl-ethyl)butanediamide (330 mg, 1.32 mmol, 97.91% yield) as a white solid.

### Step 7

To a solution of (2R)-2-amino-N-(1-methyl-2-phenyl-ethyl)butanediamide (330 mg, 1.32 mmol, 1 eq, TFA) in DMF (5 mL) was added DIPEA (513.21 mg, 3.97 mmol, 691.66 uL, 3 eq) and octanoyl octanoate (536.90 mg, 1.99 mmol, 1.5 eq). The mixture was stirred at 25° C. for 1 hr. The reaction mixture was diluted with H₂O 20 mL and filtered and filter cake concentrated under reduced pressure to give a residue. The crude product was triturated with H₂O (20 mL) and EtOAc (30 mL) at 25° C. for 30 min, filtered and filter cake concentrated under reduced pressure to give a residue. The crude product was triturated with PE:EA=2:1 (21 mL) at 25° C. for 20 min and filtered and filter cake concentrated under reduced pressure to give compound (2R)-N-(1-methyl-2-phenyl-ethyl)-2-(octanoylamino)butanediamide (327 mg, 853.41 umol, 64.47% yield, 98% purity) as a white solid.

### Step 8

Compound (2R)-N-(1-methyl-2-phenyl-ethyl)-2-(octanoylamino)butanediamide was separated by SFC (column: DAICEL CHIRALPAK IG (250 mm*30 mm, 10 um); mobile phase: [Neu-ETOH]; B %: 40%-40%, min) to give (2R)-N-[(1R)-1-methyl-2-phenyl-ethyl]-2-(octanoylamino)butanediamide (106 mg, 265.35 umol, 33.21% yield, 94% purity) as a white solid.

(2R)-N-[(1S)-1-methyl-2-phenyl-ethyl]-2-(octanoylamino)butanediamide (118 mg, 298.53 umol, 37.37% yield, 95% purity) was obtained as a white solid.

### Example 176: (R)-2-octanamido-N1-(2-(pyridin-2-yl)ethyl)succinamide

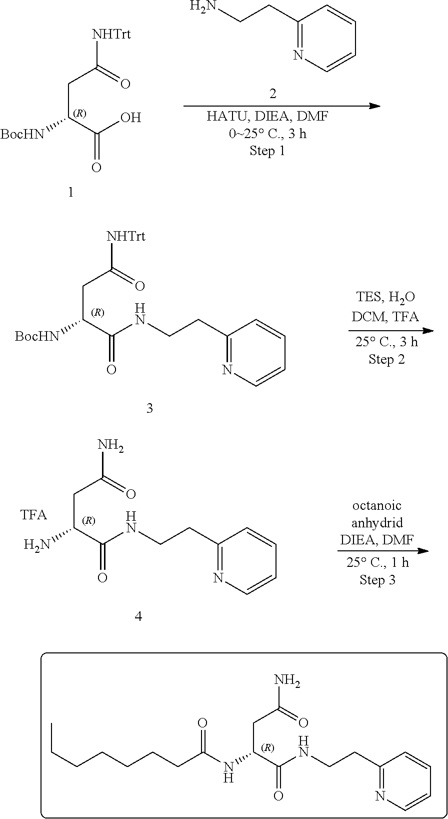

### Step 1

To a solution of (2R)-2-(tert-butoxycarbonylamino)-4-oxo-4-(tritylamino)butanoic acid (1 g, 2.11 mmol, 1 eq) and 2-(2-pyridyl)ethanamine (308.93 mg, 2.53 mmol, 302.87 uL, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 3 h. LC-MS showed the desired compound was detected. The reaction mixture was poured to 100 mL H₂O and then the mixture was filtered and the filter cake was concentrated under reduced pressure to give a residue. The product tert-butyl N-[(1R)-3-oxo-1-[2-(2-pyridyl)ethylcarbamoyl]-3-(tritylamino)propyl]carbamate (700 mg, 1.17 mmol, 55.68% yield, 97% purity) as white solid.

### Step 2

A mixture of triethylsilane (364.00 mg, 3.13 mmol, 0.5 mL, 3.02 eq), H₂O (500.00 mg, 27.75 mmol, 0.5 mL, 26.77 eq), DCM (1.32 g, 15.54 mmol, 1 mL, 14.99 eq) and TFA (27.72 g, 243.11 mmol, 18 mL, 234.48 eq) was added tert-butyl N-[(1R)-3-oxo-1-[2-(2-pyridyl)ethylcarbamoyl]-3-(tritylamino)propyl]carbamate (600 mg, 1.04 mmol, 1 eq). Then the mixture was stirred at 25° C. for 3 h under N₂ atmosphere. LC-MS showed the desired compound was detected. The reaction mixture was concentrated under reduced pressure to give a crude product (2R)-2-amino-N-[2-(2-pyridyl)ethyl]butanediamide (400 mg, crude, TFA) as white solid.

### Step 3

To a solution of (2R)-2-amino-N-[2-(2-pyridyl)ethyl]butanediamide (400 mg, 1.14 mmol, 1 eq, TFA) in DMF (10 mL) was added DIEA (442.75 mg, 3.43 mmol, 596.70 uL, 3 eq) and octanoyl octanoate (370.54 mg, 1.37 mmol, 1.2 eq). The mixture was stirred at 25° C. for 1 h. LC-MS showed the desired compound was detected. The reaction mixture was concentrated under reduced pressure to give a residue. The residue was purified by prep-HPLC ([water(10 mM NH₄HCO₃)-ACN]) to give compound (2R)-2-(octanoylamino)-N-[2-(2-pyridyl) ethyl]butanediamide(75 mg, 0.19 mmol, 10% yield) as white solid.

### Example 177: (R)-2-octanamido-N′-(2-(thiophen-3-yl)ethyl)succinamide

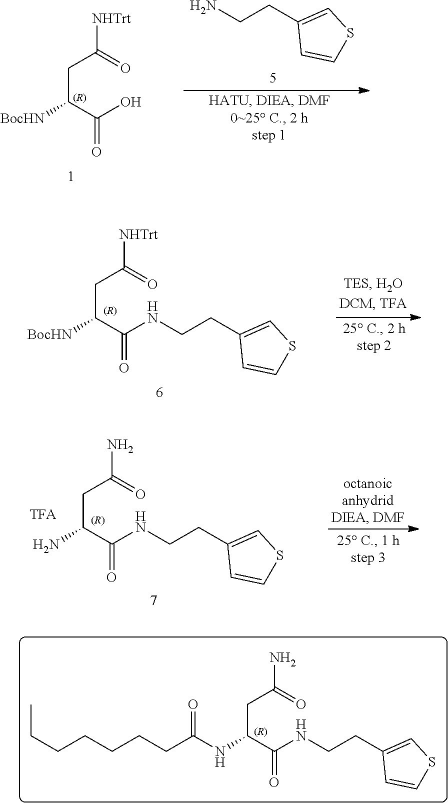

### Step 1

To a solution of (2R)-2-(tert-butoxycarbonylamino)-4-oxo-4-(tritylamino)butanoic acid (1 g, 2.11 mmol, 1 eq) and 2-(3-thienyl)ethanamine (413.87 mg, 2.53 mmol, 1.2 eq, HCl) in DMF (10 mL) was added HATU (961.50 mg, 2.53 mmol, 1.2 eq) and DIPEA (817.05 mg, 6.32 mmol, 1.10 mL, 3 eq) at 0° C. The mixture was stirred at 25° C. for 2 hr. The reaction mixture was quenched by addition H₂O 30 mL at 0° C. The precipitate was filtered and the filter cake was concentrated under reduced pressure to give compound tert-butyl N-[(1R)-3-oxo-1-[2-(3-thienyl)ethylcarbamoyl]-3-(tritylamino) propyl]carbamate (1 g, 1.64 mmol, 78.04% yield, 96% purity) as a white solid.

### Step 2

The tert-butyl N-[(1R)-3-oxo-1-[2-(3-thienyl)ethylcarbamoyl]-3-(tritylamino)propyl]carbamate (800 mg, 1.37 mmol, 1 eq) in TES (0.2 mL), H₂O (0.2 mL), DCM (0.4 mL) and TFA (7.2 mL) was stirred at 25° C. for 2 hr. The mixture was concentrated under reduced pressure to give compound (2R)-2-amino-N-[2-(3-thienyl)ethyl]butanediamide (486 mg, crude, TFA) as a white solid.

### Step 3

To a solution of (2R)-2-amino-N-[2-(3-thienyl)ethyl]butanediamide (480 mg, 1.35 mmol, 1 eq, TFA) and octanoyl octanoate (547.92 mg, 2.03 mmol, 1.5 eq) in DMF (5 mL) was added DIPEA (523.76 mg, 4.05 mmol, 705.88 uL, 3 eq). The mixture was stirred at 25° C. for 1 hr. The mixture was concentrated under reduced pressure to give a residue. The residue was purified by prep-HPLC (column: Phenomenex Luna C18 100*30 mm*5 um; mobile phase: [water (0.1% TFA)-ACN]; B %: 30%-60%, 10 min) to give compound (2R)-2-(octanoylamino)-N-[2-(3-thienyl)ethyl]butanediamide (70 mg, 190.47 umol, 14.10% yield, 100% purity) as a white solid.

### Example 178: (R)-N-(3-amino-1-(1H-benzo[d]imidazol-2-yl)-3-oxopropyl)octanamide

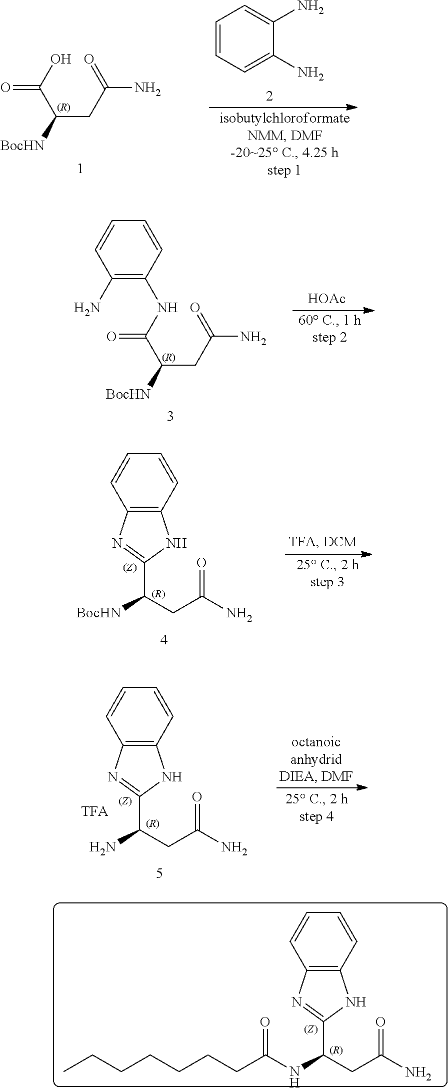

### Step 1

To a mixture of (2R)-4-amino-2-(tert-butoxycarbonylamino)-4-oxo-butanoic acid (10 g, 43.06 mmol, 1 eq) and NMM (4.36 g, 43.06 mmol, 4.73 mL, 1 eq) in DMF (60 mL) was added isobutyl carbonochloridate (5.88 g, 43.06 mmol, 5.65 mL, 1 eq) dropwise at −20° C. under N₂. The mixture was stirred at −20° C. for 15 min. Then benzene-1,2-diamine (4.66 g, 43.06 mmol, 1 eq) was added. The mixture was allowed to slowly warming to 25° C. over 1 h and then stirred for 3 hours. The reaction mixture was concentrated under reduced pressure to give a residue. To the residue was added water (100 mL) and the mixture was extracted with ethyl acetate (100 mL*2). The combined organic phase was washed with 5% NaHCO₃ solution (100 mL) and brine (100 mL), dried with anhydrous Na₂SO₄, filtered and concentrated in vacuum. The residue was triturated with (Petroleum ether: Ethyl acetate=1/1, 40 mL) at 25° C. for 10 min. The mixture was filtered and the filter cake was concentrated in vacuum to get tert-butyl N-[(1R)-3-amino-1-[(2-aminophenyl) carbamoyl]-3-oxo-propyl]carbamate (3.03 g, 7.43 mmol, 17.24% yield, 79% purity) as a yellow solid.

### Step 2

A mixture of tert-butyl N-[(1R)-3-amino-1-[(2-aminophenyl)carbamoyl]-3-oxo-propyl]carbamate (1 g, 3.10 mmol, 1 eq) in AcOH (10 mL) was stirred at 60° C. for 1 hour. LCMS showed the reaction was completed. The mixture was concentrated in vacuum. The crude product was triturated with (Petroleum ether: Ethyl acetate=1/1) at 25° C. for 10 min. The mixture was filtered and the filter cake was concentrated in vacuum to get tert-butyl N-[(1R)-3-amino-1-(1H-benzimidazol-2-yl)-3-oxo-propyl]carbamate (500 mg, 1.47 mmol, 47.37% yield, 89.44% purity) as a yellow solid.

### Step 3

A mixture of tert-butyl N-[(1R)-3-amino-1-(1H-benzimidazol-2-yl)-3-oxo-propyl]carbamate (500 mg, 1.47 mmol, 89.44% purity, 1 eq) in DCM (40 mL) and TFA (10 mL) was stirred at 25° C. for 2 hours. The mixture was concentrated in reduced pressure to get (3R)-3-amino-3-(1H-benzimidazol-2-yl) propanamide (450 mg, crude, TFA) as a yellow solid.

### Step 4

To a mixture of (3R)-3-amino-3-(1H-benzimidazol-2-yl)propanamide (450 mg, 1.41 mmol, 1 eq, TFA) and octanoyl octanoate (573.52 mg, 2.12 mmol, 1.5 eq) in DMF (10 mL) was added DIPEA (913.73 mg, 7.07 mmol, 1.23 mL, 5 eq) in one portion at 25° C. under N₂. The mixture was stirred at 25° C. for 2 hours. LCMS showed the reaction was completed and desired mass was detected. To the mixture was added water (50 mL) and the mixture was extracted with ethyl acetate (50 mL*3). The combined organic phase was washed with brine (100 mL), dried with anhydrous Na₂SO₄, filtered and concentrated in vacuum. The residue was purified by p-HPLC (column: Phenomenex Gemini-NX C18 75*30 mm*3 um; mobile phase: [water(10 mM NH4HCO3)-ACN]; B %: 15%-45%, 6 min) to give N-[(1R)-3-amino-1-(1H-benzimidazol-2-yl)-3-oxo-propyl]octanamide (64 mg, 186.81 umol, 13.21% yield, 96.45% purity) as a white solid.

### Example 179: (R)-2-octanamido-N′-(2-(pyridin-4-yl)ethyl)succinamide

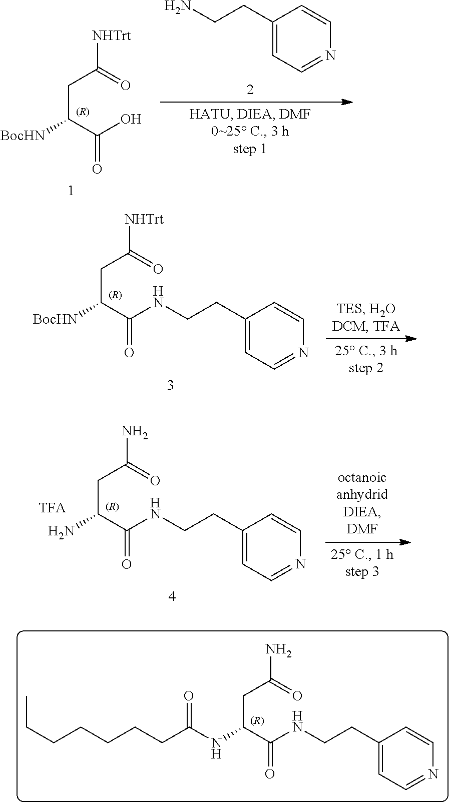

### Step 1

To a mixture of (2R)-2-(tert-butoxycarbonylamino)-4-oxo-4-(tritylamino)butanoic acid (2 g, 4.21 mmol, 1 eq) and 2-(4-pyridyl)ethanamine (617.86 mg, 5.06 mmol, 611.74 uL, 1.2 eq) in DMF (60 mL) was added HATU (1.92 g, 5.06 mmol, 1.2 eq) and DIPEA (1.20 g, 9.27 mmol, 1.62 mL, 2.2 eq) at 0° C. under N2. The mixture was stirred at 25° C. for 3 hours. LCMS showed the reaction was completed and desired mass was detected. The mixture was cooled to 0° C. and water (120 mL) was added slowly, then the precipitate was filtered and the filter cake was washed by water (30 mL*2). The filter cake was concentrated in vacuum to give compound tert-butyl N-[(1R)-3-oxo-1-[2-(4-pyridyl)ethylcarbamoyl]-3-(tritylamino)propyl]carbamate (2.1 g, 3.59 mmol, 85.09% yield, 98.82% purity) as a white solid.

### Step 2

The mixture of tert-butyl N-[(1R)-3-oxo-1-[2-(4-pyridyl)ethylcarbamoyl]-3-(tritylamino)propyl]carbamate (2 g, 3.46 mmol, 1 eq), TES (792.29 mg, 3.46 mmol, 1.25 mL, 1 eq), H2O (1.25 g, 69.37 mmol, 1.25 mL, 20.07 eq), DCM (3.30 g, 38.86 mmol, 2.50 mL, 11.24 eq) and TFA (69.30 g, 607.79 mmol, 45.00 mL, 175.86 eq) was stirred at 25° C. for 3 hr. TLC showed two new spots formed. The reaction mixture was concentrated to give a compound (2R)-2-amino-N-[2-(4-pyridyl)ethyl]butanediamide (1.5 g, crude, TFA) as a yellow solid.

### Step 3

To a solution of (2R)-2-amino-N-[2-(4-pyridyl)ethyl]butanediamide (1.21 g, 3.45 mmol, 1 eq, TFA) and octanoyl octanoate (1.40 g, 5.18 mmol, 1.5 eq) in DMF (25 mL) was added DIPEA (1.34 g, 10.36 mmol, 1.81 mL, 3 eq). The mixture was stirred at 25° C. for 1 hr. The reaction mixture was treated with water (40 mL), and then extracted with EtOAc (50 mL*3). The combined organic phase was washed with brine (40 mL), dried with anhydrous Na₂SO₄, filtered and concentrated in vacuum to give compound (2R)-2-(octanoylamino)-N-[2-(4-pyridyl)ethyl]butanediamide (300 mg, 816.91 umol, 23.65% yield, 98.7% purity) as a white solid.

### Example 180: Benzyl (S)-(4-amino-1-(heptylamino)-1,4-dioxobutan-2-yl)carbamate

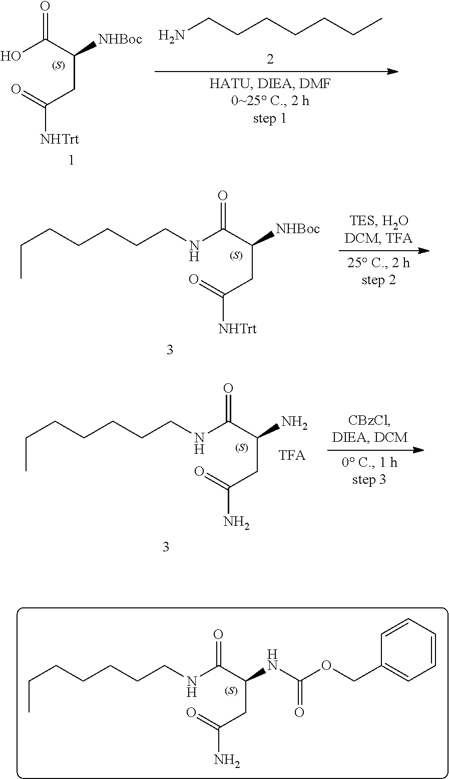

### Step 1

To a solution of (2S)-2-(tert-butoxycarbonylamino)-4-oxo-4-(tritylamino)butanoic acid (3 g, 6.32 mmol, 1 eq) and heptan-1-amine (874.05 mg, 7.59 mmol, 1.13 mL, 1.2 eq) in DMF (30 mL) was added HATU (2.88 g, 7.59 mmol, 1.2 eq) and DIPEA (1.80 g, 13.91 mmol, 2.42 mL, 2.2 eq) at 0° C. The mixture was stirred at 25° C. for 2 hr. TLC showed the starting reactant was consumed and one new spot was formed. The reaction mixture was quenched by addition H₂O 50 mL at 0° C. and the mixture was filtered and the filter cake was concentrated under reduced pressure to give compound tert-butyl N-[(1S)-1-(heptylcarbamoyl)-3-oxo-3-(tritylamino)propyl]carbamate (3 g, crude) as a white solid.

### Step 2

The tert-butyl N-[(1S)-1-(heptylcarbamoyl)-3-oxo-3-(tritylamino)propyl]carbamate (1.5 g, 2.62 mmol, 1 eq) in TES (0.5 mL), H₂O (0.5 mL), DCM (1 mL) and TFA (18 mL) was stirred at 25° C. for 2 hr. The mixture was concentrated under reduced pressure to give compound (2S)-2-amino-N-heptyl-butanediamide (900 mg, crude, TFA) as a white solid.

### Step 3

To a solution of (2S)-2-amino-N-heptyl-butanediamide (300 mg, 873.77 umol, 1 eq, TFA) in DCM (5 mL) was added DIPEA (225.86 mg, 1.75 mmol, 304.39 uL, 2 eq) and benzyl carbonochloridate (163.96 mg, 961.14 umol, 136.64 uL, 1.1 eq). The mixture was stirred at 0° C. for 1 hr. The mixture was concentrated under reduced pressure to give a residue. The residue was purified by prep-HPLC (column: Phenomenex Luna C18 100*30 mm*5 um; mobile phase: [water (0.1% TFA)-ACN]; B %: 30%-60%, 10 min) to give compound benzyl N-[(1S)-3-amino-1-(heptylcarbamoyl)-3-oxo-propyl]carbamate (88 mg, 242.12 umol, 27.71% yield) as a white solid.

### Example 181: Phenyl (S)-(4-amino-1-(heptylamino)-1,4-dioxobutan-2-yl)carbamate

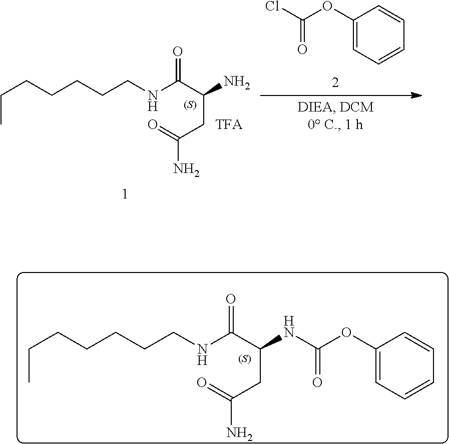

To a solution of (2S)-2-amino-N-heptyl-butanediamide (300 mg, 873.77 umol, 1 eq, TFA) in DCM (5 mL) was added DIPEA (225.86 mg, 1.75 mmol, 304.39 uL, 2 eq). Then phenyl carbonochloridate (150.48 mg, 961.14 umol, 120.39 uL, 1.1 eq) was added at 0° C. The mixture was stirred at 0° C. for 1 hr. The mixture was concentrated under reduced pressure to give a residue. The residue was purified by prep-HPLC (column: Phenomenex Luna C18 100*30 mm*5 um; mobile phase: [water(0.1% TFA)-ACN]; B %: 30%-60%, 10 min) to give compound phenyl N-[(1S)-3-amino-1-(heptylcarbamoyl)-3-oxo-propyl]carbamate (97 mg, 277.60 umol, 31.77% yield) as a white solid.

### Example 182: (R)-N-(3-amino-3-oxo-1-(5-phenethyl-1,3,4-oxadiazol-2-yl)propyl)octanamide

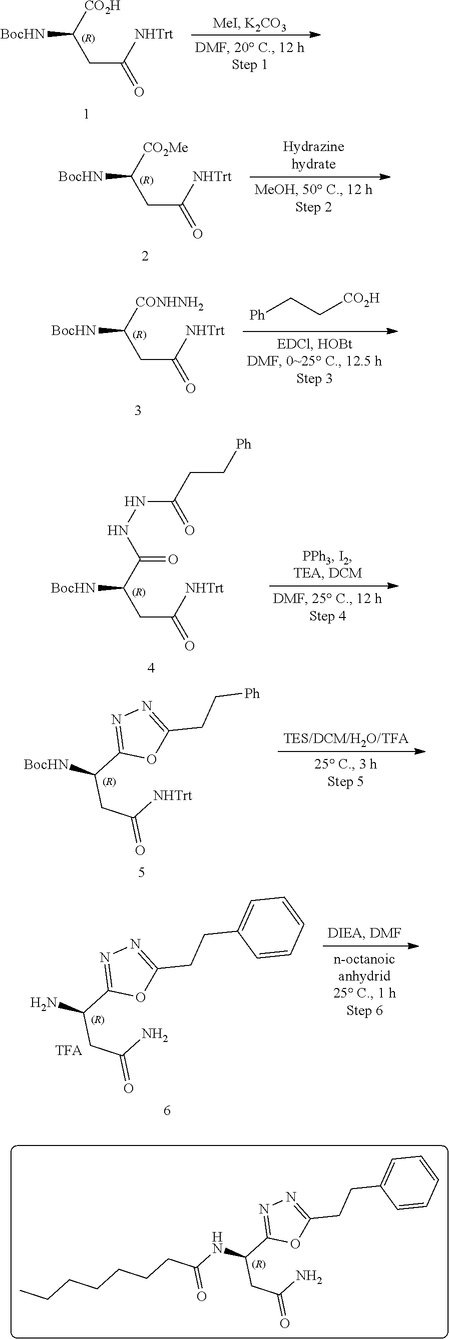

### Step 1

To a solution of (2R)-2-(tert-butoxycarbonylamino)-4-oxo-4-(tritylamino)butanoic acid (5 g, 10.54 mmol, 1 eq) in DMF (50 mL) was added K₂CO₃ (2.18 g, 15.80 mmol, 1.5 eq) and iodomethane (1.79 g, 12.64 mmol, 787.11 uL, 1.2 eq). The mixture was stirred at 20° C. for 12 hr. TLC indicated reactant was consumed completely. The reaction mixture was poured to water (100 mL) and then the mixture was filtered and the filter cake was concentrated under reduced pressure to give compound methyl (2R)-2-(tert-butoxycarbonylamino)-4-oxo-4-(tritylamino)butanoate (4 g, 8.19 mmol, 77.70% yield) as white solid.

### Step 2

To a solution of methyl (2R)-2-(tert-butoxycarbonylamino)-4-oxo-4-(tritylamino)butanoate (2 g, 4.09 mmol, 1 eq) in MeOH (20 mL) was added hydrazine hydrate (1.25 g, 24.56 mmol, 1.22 mL, 98% purity, 6 eq). The mixture was stirred at 50° C. for 12 h. TLC indicated reactant was consumed completely. The reaction mixture was concentrated under reduced pressure to remove MeOH. The residue was diluted with DCM/MeOH=10/1 200 mL and extracted with H₂O 150 mL (50 mL*3). The combined organic layers were washed with brine 100 mL (50 mL*2), dried over Na₂SO₄, filtered and the filtrate was concentrated under reduced pressure to give the product tert-butyl N-[(1R)-1-(hydrazinecarbonyl)-3-oxo-3-(tritylamino)propyl]carbamate (1.7 g, 3.48 mmol, 85.00% yield) as white solid.

### Step 3

To a solution of 3-phenylpropanoic acid (400 mg, 2.66 mmol, 373.83 uL, 1 eq) in DMF (10 mL) was added HOBt (377.91 mg, 2.80 mmol, 1.05 eq) and EDCI (536.14 mg, 2.80 mmol, 1.05 eq) for 30 min, then tert-butyl N-[(1R)-1-(hydrazinecarbonyl)-3-oxo-3-(tritylamino)propyl]carbamate (1.30 g, 2.66 mmol, 1 eq) was added to the mixture and the mixture was stirred at 25° C. for 12 h. TLC indicated 3-phenylpropanoic acid was consumed completely. The reaction mixture was partitioned between H₂O and EtOAc. The organic phase was separated, washed with brine 60 ml (30 mL*2), dried over Na₂SO₄, filtered and the filtrate was concentrated under reduced pressure to give compound tert-butyl N-[(1R)-3-oxo-1-[(3-phenylpropanoylamino)carbamoyl]-3-(tritylamino)propyl]carbamate (1.1 g, 1.77 mmol, 66.53% yield) as white solid.

### Step 4

To a solution of triphenylphosphane (422.54 mg, 1.61 mmol, 2 eq) in DCM (5 mL) was added I₂ (408.88 mg, 1.61 mmol, 324.51 uL, 2 eq) for 5 min, then TEA (342.33 mg, 3.38 mmol, 470.88 uL, 4.2 eq) and tert-butyl N-[(1R)-3-oxo-1-[(3-phenylpropanoylamino)carbamoyl]-3-(tritylamino)propyl]carbamate (500 mg, 805.50 umol, 1 eq) in DMF (5 mL) were added to the mixture and the mixture was stirred at 25° C. for 12 h. The reaction mixture was partitioned between H₂O and EtOAc, The organic phase was separated, washed with brine 60 ml (20 mL*2), dried over Na₂SO₄, filtered and the filtrate was concentrated under reduced pressure to give a residue. The residue was purified by prep-TLC (SiO₂, PE: EtOAc=1:1) to give compound tert-butylN-[(1R)-3-oxo-1-[5-(2-phenylethyl)-1,3,4-oxadiazol-2-yl]-3-(tritylamino)propyl]carbamate (160 mg, 254.84 umol, 31.64% yield, 96% purity) as white solid.

### Step 5

A mixture of triethylsilane (72.80 mg, 626.09 umol, 0.1 mL, 2.36 eq), H₂O (100.00 mg, 5.55 mmol, 0.1 mL, 20.91 eq), DCM (264.00 mg, 3.11 mmol, 0.2 mL, 11.71 eq) and TFA (5.54 g, 48.62 mmol, 3.6 mL, 183.16 eq) was added tert-butyl N-[(1R)-3-oxo-1-[5-(2-phenylethyl)-1,3,4-oxadiazol-2-yl]-3-(tritylamino)propyl]carbamate (160 mg, 265.46 umol, 1 eq) and the mixture was stirred at 25° C. for 3 hr under N₂ atmosphere. The reaction mixture was concentrated under reduced pressure to give the product (3R)-3-amino-3-[5-(2-phenylethyl)-1,3,4-oxadiazol-2-yl]propanamide (80 mg, 213.72 umol, 80.51% yield, TFA) as white solid.

### Step 6

To a solution of (3R)-3-amino-3-[5-(2-phenylethyl)-1,3,4-oxadiazol-2-yl]propanamide (80 mg, 213.72 umol, 1 eq, TFA) in DMF (5 mL) was added DIPEA (82.87 mg, 641.17 umol, 111.68 uL, 3 eq) and octanoyl octanoate (86.69 mg, 320.59 umol, 1.5 eq). The mixture was stirred at 25° C. for 1 hr. The reaction mixture was concentrated under reduced pressure to give a residue. The residue was purified by prep-HPLC ([water (10 mM NH₄HCO₃)-ACN]) to give compound N-[(1R)-3-amino-3-oxo-1-[5-(2-phenylethyl)-1,3,4-oxadiazol-2-yl]propyl]octanamide (37 mg, 95.73 umol, 44.79% yield) as white solid.

### Example 183: (R)-2-(2,2-dimethyloctanamido)-N′-phenethylsuccinamide

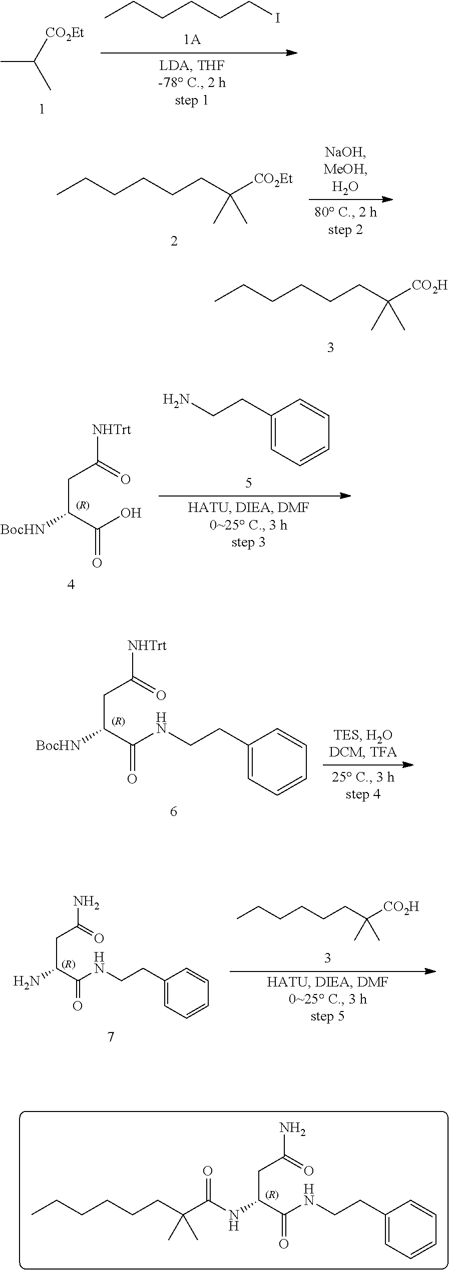

### Step 1

To a solution of ethyl 2-methylpropanoate (2 g, 17.22 mmol, 2.31 mL, 1 eq) in THF (17 mL) was added LDA (2 M, 2.48 mL, 0.288 eq) at −78° C. under N₂, and the reaction mixture was stirred for 30 min. Then 1-iodohexane (4.02 g, 18.94 mmol, 2.79 mL, 1.1 eq) was added to the mixture, and the mixture was stirred at −78° C. for 1.5 hr. TLC showed new spots formed. The reaction mixture was treated with 1 N HCl aq (30 mL), and then extracted with EtOAc (50 mL*3), dried with anhydrous Na₂SO₄, filtered and concentrated in vacuum to give compound ethyl 2,2-dimethyloctanoate (2.4 g, crude) as a brown oil.

¹H NMR (400 MHz, CDCl₃)

### Step 2

To a mixture of ethyl 2,2-dimethyloctanoate (2.3 g, 11.48 mmol, 1 eq), MeOH (15 mL) and H₂O (15 mL) was added NaOH (1.38 g, 34.45 mmol, 3 eq), and the mixture was stirred at 80° C. for 2 hr. New spot was detected by TLC. The reaction mixture treated with 1 N HCl aq to pH=1, and extracted with EtOAc (60 mL*3), dried with anhydrous Na₂SO₄, filtered and concentrated in vacuum. The residue was purified by flash silica gel chromatography (Silica Flash Column, Eluent of 15˜20% Ethyl acetate/Petroleum ether gradient) to give compound 2,2-dimethyloctanoic acid (300 mg, crude) as a yellow oil.

### Step 3

To a mixture of (2R)-2-(tert-butoxycarbonylamino)-4-oxo-4-(tritylamino)butanoic acid (3 g, 6.32 mmol, 1 eq) and 2-phenylethanamine (919.29 mg, 7.59 mmol, 952.63 uL, 1.2 eq) in DMF (90 mL) was added HATU (2.88 g, 7.59 mmol, 1.2 eq) and DIPEA (1.80 g, 13.91 mmol, 2.42 mL, 2.2 eq) at 0° C. under N₂. The mixture was stirred at 25° C. for 3 hours. The mixture was cooled to 0° C. and water (150 mL) was added slowly, then the precipitate was filtered and the filter cake was washed by water (50 mL*2). The filter cake was concentrated in vacuum to give compound tert-butyl N-[(1R)-3-oxo-1-(2-phenylethylcarbamoyl)-3-(tritylamino)propyl]carbamate (6.4 g, 11.08 mmol, 87.62% yield) as a white solid.

### Step 4

A mixture of tert-butyl N-[(1R)-3-oxo-1-(2-phenylethylcarbamoyl)-3-(tritylamino)propyl]carbamate (3.2 g, 5.54 mmol, 1 eq) in TFA (107.80 g, 945.45 mmol, 70 mL, 170.69 eq), DCM (5.28 g, 62.17 mmol, 4 mL, 11.22 eq), H₂O (2.00 g, 110.99 mmol, 2 mL, 20.04 eq), triethylsilane (1.46 g, 12.52 mmol, 2 mL, 2.26 eq) was stirred at 25° C. for 3 h. LCMS showed the reaction was completed and desired mass was detected. The mixture was concentrated in vacuum. Then the mixture was treated by sat. NaHCO₃ (150 mL) aq. to pH=8. The aqueous phase was extracted with ethyl acetate (100 mL*2). Then the aqueous phase was concentrated in vacuum. The residue was purified by silica gel chromatography (silica gel, Petroleum ether/Ethyl acetate=1/1, 0/1 to THF/MeOH=1/0, 3/1) to give compound (2R)-2-amino-N-(2-phenylethyl) butanediamide (2.2 g, 8.79 mmol, 79.34% yield, 94% purity) as a white solid.

### Step 5

To a solution of 2,2-dimethyloctanoic acid (231.15 mg, 1.34 mmol, 9.71e-1 eq) and (2R)-2-amino-N-(2-phenylethyl)butanediamide (390 mg, 1.66 mmol, 1.2 eq) in DMF (5 mL) was added HATU (630.26 mg, 1.66 mmol, 1.2 eq) and DIPEA (214.23 mg, 1.66 mmol, 288.72 uL, 1.2 eq) at 0° C. And the mixture was stirred at 25° C. for 3 hr. The desired mass was detected by LCMS. The reaction mixture was treated with water (20 mL), and extracted with EtOAc (30 mL*3). The combined organic phase was washed with brine (30 mL), dried with anhydrous Na₂SO₄, filtered and concentrated in vacuum. The reaction mixture was purified by prep-HPLC (column: Phenomenex Gemini-NX 80*40 mm*3 um; mobile phase: [water(10 mM NH₄HCO₃)-ACN]; B %: 25%-55%, 8 min) to give compound (2R)-2-(2,2-dimethyloctanoylamino)-N-(2-phenylethyl) butanediamide (17 mg, 44.98 umol, 3.26% yield, 99% purity) as a white solid.

### Example 184: (R)-2-octanamido-N′-(3-phenylpropyl)succinamide

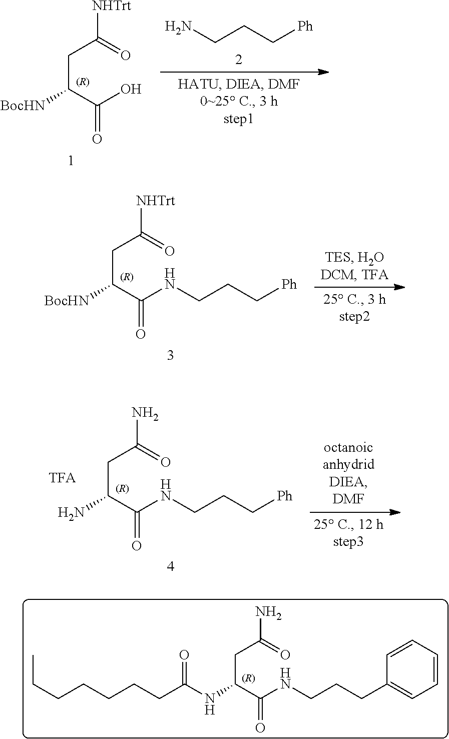

### Step 1

To a solution of (2R)-2-(tert-butoxycarbonylamino)-4-oxo-4-(tritylamino)butanoic acid (1 g, 2.11 mmol, 1 eq) and 3-phenylpropan-1-amine (341.90 mg, 2.53 mmol, 361.03 uL, 1.2 eq) in DMF (10 mL) was added HATU (961.50 mg, 2.53 mmol, 1.2 eq) and DIEA (599.16 mg, 4.64 mmol, 807.49 uL, 2.2 eq) at 0° C. The mixture was stirred at 25° C. for 3 hr. TLC showed the starting reactant was consumed and one new spots formed. The reaction mixture was quenched by addition H₂O 60 mL at 0° C., and the precipitate was filtered and filter cake was concentrated under reduced pressure. Compound tert-butyl N-[(1R)-3-oxo-1-(3-phenylpropylcarbamoyl)-3-(tritylamino)propyl]carbamate (1 g, 1.57 mmol, 74.58% yield, 93% purity) was obtained as a white solid without purification.

### Step 2

A mixture of tert-butyl N-[(1R)-3-oxo-1-(3-phenylpropylcarbamoyl)-3-(tritylamino)propyl]carbamate (0.9 g, 1.52 mmol, 1 eq), TES (871.69 mg, 3.80 mmol, 0.5 mL, 2.5 eq), H₂O (562.50 mg, 31.22 mmol, 562.50 uL, 20.52 eq), DCM (1.49 g, 17.48 mmol, 1.13 mL, 11.50 eq) and TFA (31.18 g, 273.50 mmol, 20.25 mL, 179.83 eq) stirred at 25° C. for 3 hr under N₂ atmosphere. TLC indicated tert-butyl N-[(1R)-3-oxo-1-(3-phenylpropylcarbamoyl)-3-(tritylamino)propyl]carbamate was consumed completely and one new spot formed. The reaction mixture was concentrated under reduced pressure to give compound (2R)-2-amino-N-(3-phenylpropyl)butanediamide (500 mg, crude, TFA) as a white solid.

### Step 3

A mixture of (2R)-2-amino-N-(3-phenylpropyl)butanediamide (0.5 g, 1.38 mmol, 1 eq, TFA) in DMF (5 mL) was added DIEA (533.56 mg, 4.13 mmol, 719.09 uL, 3 eq), octanoyl octanoate (558.18 mg, 2.06 mmol, 1.5 eq) and purged with N2 for 3 times. The mixture was stirred at 25° C. for 12 hr under N₂ atmosphere. The mixture was filtered and the filter cake was concentrated under reduced pressure to give a residue. The residue was purified by prep-HPLC (column: Phenomenex luna C18 100*40 mm*5 um; mobile phase: [water(0.1% TFA)-ACN]; B %:1%-30%, 8 min) to give compound (2R)-2-(octanoylamino)-N-(3-phenylpropyl) butanediamide(100 mg, 266.31 umol, 19.35% yield, 100% purity) as a white solid.

### Example 185: (2R)-N′-phenethyl-2-((1,1,1-trifluorononan-2-yl)amino)succinamide

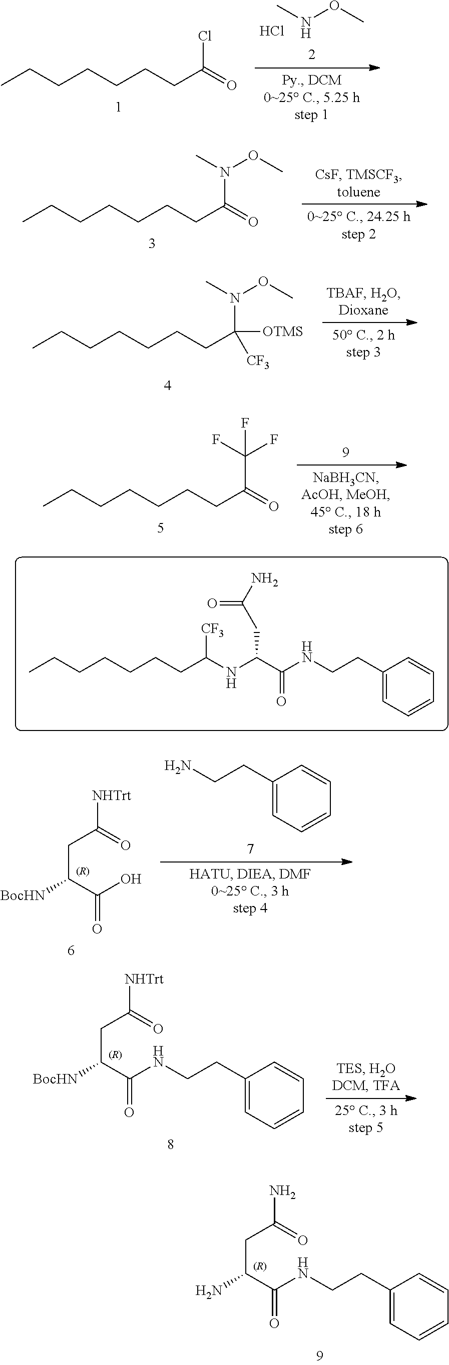

### Step 1

To a solution of hydrogen chloride (6.60 g, 67.63 mmol, 6.47 mL, 1.1 eq, HCl) and pyridine (10.70 g, 135.25 mmol, 10.92 mL, 2.2 eq) in DCM (100 mL) was added octanoyl chloride (10 g, 61.48 mmol, 10.49 mL, 1 eq) drop-wise at 0° C. over a period of 15 mins under N2. The reaction mixture was warmed to 25° C. and stirred for 5 hours. The mixture was diluted with DCM (100 mL), washed with 1N HCl (150 mL*2), sat·NaHCO₃ (100 mL*2) and brine (100 mL). The organic phase was dried over anhydrous Na₂SO₄, filtered and concentrated in vacuo to get N-methoxy-N-methyloctanamide (11 g, 54.72 mmol, 89.01% yield, 93.17% purity) as colorless oil.

¹H NMR (400 MHz, CDCl3)

### Step 2

A solution of N-methoxy-N-methyl-octanamide (10 g, 53.40 mmol, 1 eq) and CsF (1.62 g, 10.68 mmol, 393.74 uL, 0.2 eq) in toluene (100 mL) was stirred at 0° C. for 15 min. Then TMSCF₃ (15.19 g, 106.79 mmol, 2 eq) was added at 0° C. under N2. The reaction mixture was warmed to 25° C. and stirred at 25° C. for 24 hours. TLC showed the reaction was completed. The mixture was concentrated in vacuum to give 1,1,1-trifluoro-N-methoxy-N-methyl-2-trimethylsilyloxy-nonan-2-amine (15 g, crude) as yellow oil.

### Step 3

To a mixture of 1,1,1-trifluoro-N-methoxy-N-methyl-2-trimethylsilyloxy-nonan-2-amine (15 g, 45.53 mmol, 1 eq) in H₂O (50 mL) and Hexane (40 mL) was added TBAF (1 M, 25.00 mL, 5.49e-1 eq) in one portion under N₂. The mixture was stirred at 50° C. for 2 hours. The mixture was cooled to 25° C. and extracted with ethyl acetate (150 mL*2). The combined organic phase was washed with brine (200 mL), dried with anhydrous Na₂SO₄, filtered and concentrated in vacuum under 30° C. The crude product was purified by distilled in vacuum (50° C. oil temperature, water pump) to get product 1,1,1-trifluorononan-2-one (500 mg, 2.55 mmol, 5.60% yield) as colorless oil.

### Step 4

To a mixture of (2R)-2-(tert-butoxycarbonylamino)-4-oxo-4-(tritylamino)butanoic acid (3 g, 6.32 mmol, 1 eq) and 2-phenylethanamine (919.29 mg, 7.59 mmol, 952.63 uL, 1.2 eq) in DMF (90 mL) was added HATU (2.88 g, 7.59 mmol, 1.2 eq) and DIPEA (1.80 g, 13.91 mmol, 2.42 mL, 2.2 eq) at 0° C. under N₂. The mixture was stirred at 25° C. for 3 hours. The mixture was cooled to 0° C. and water (150 mL) was added slowly. Then the precipitate was filtered and the filter cake was washed by water (50 mL*2). The filter cake was concentrated in vacuum to get tert-butyl N-[(1R)-3-oxo-1-(2-phenylethylcarbamoyl)-3-(tritylamino)propyl]carbamate (6.4 g, 11.08 mmol, 87.62% yield) as a white solid.

### Step 5

A mixture of tert-butyl N-[(1R)-3-oxo-1-(2-phenylethylcarbamoyl)-3-(tritylamino)propyl]carbamate (3.2 g, 5.54 mmol, 1 eq), TFA (107.80 g, 945.45 mmol, 70 mL, 170.69 eq), DCM (5.28 g, 62.17 mmol, 4 mL, 11.22 eq), H₂O (2.00 g, 110.99 mmol, 2 mL, 20.04 eq), triethylsilane (1.46 g, 12.52 mmol, 2 mL, 2.26 eq) was stirred at 25° C. for 3 h. LCMS showed the reaction was completed and desired mass was detected. The mixture was concentrated in vacuum. Then the mixture was treated by sat·NaHCO₃ (150 mL) to pH=8. The aqueous phase was extracted with ethyl acetate (100 mL*2). Then the aqueous phase was concentrated in vacuum to give a residue. The residue was purified by silica gel chromatography (silica gel, Petroleum ether/Ethyl acetate=1/1 to THF/MeOH=3/1) to get (2R)-2-amino-N-(2-phenylethyl)butanediamide (2.2 g, 8.79 mmol, 79.34% yield, 94% purity) as a white solid.

### Step 6

A mixture of (2R)-2-amino-N-(2-phenylethyl)butanediamide (200 mg, 850.05 umol, 1 eq) and 1,1,1-trifluorononan-2-one (250.18 mg, 1.28 mmol, 1.5 eq) in MeOH (3 mL) was added AcOH (102.09 mg, 1.70 mmol, 97.23 uL, 2 eq) and the mixture was stirred at 45° C. for 10 hr. Then NaBH₃CN (160.26 mg, 2.55 mmol, 3 eq) was added in one portion under N₂. The mixture was stirred at 45° C. for 8 hours. LCMS showed 19% of product was detected. The mixture was purified directly by p-HPLC(column: Phenomenex Luna C18 100*30 mm*5 um; mobile phase: [water(0.05% HCl)-ACN]; B %: 45%-80%, 10 min) to get (2R)-N-(2-phenylethyl)-2-[1-(trifluoro methyl)octylamino]butanediamide (10 mg, 99% purity) as a white solid.

### Example 186: (R)-4-amino-1,4-dioxo-1-(phenethylamino)butan-2-yl octanoate

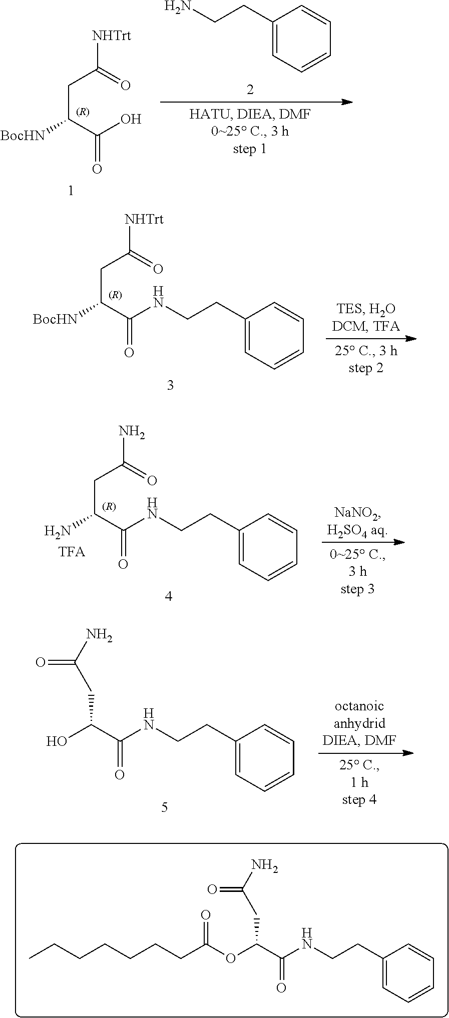

### Step 1

To a solution of (2S)-2-(tert-butoxycarbonylamino)-4-oxo-4-(tritylamino)butanoic acid (3 g, 6.32 mmol, 1 eq) and 2-phenylethanamine (919.29 mg, 7.59 mmol, 952.63 uL, 1.2 eq) in DMF (30 mL) was added HATU (2.88 g, 7.59 mmol, 1.2 eq) and DIPEA (1.80 g, 13.91 mmol, 2.42 mL, 2.2 eq) at 0° C. The mixture was stirred at 25° C. for 2 hr. TLC showed the starting reactant was consumed and one new spot was formed. The reaction mixture was quenched by addition H₂O 50 mL at 0° C., the precipitate was filtered and the filter cake was concentrated under reduced pressure to give compound tert-butyl N-[(1R)-3-oxo-1-(2-phenylethylcarbamoyl)-3-(tritylamino)propyl]carbamate (3.5 g, 5.88 mmol, 92.96% yield, 97% purity) as a white solid.

### Step 2

The mixture of tert-butyl N-[(1R)-3-oxo-1-(2-phenylethylcarbamoyl)-3-(tritylamino)propyl]carbamate (3.5 g, 6.06 mmol, 1 eq) in TES (0.5 mL), H₂O (0.5 mL), DCM (1 mL) and TFA (18 mL) was stirred at 25° C. for 3 hr. The mixture was concentrated under reduced pressure to give a residue. The residue was purified by re-crystallization with PE:EtOAc (1:1, 50 mL) at 25° C. for 30 min. Then the precipitate was filtered and the filter cake was concentrated under reduced pressure to give compound (2R)-2-amino-N-(2-phenylethyl)butanediamide (1.8 g, 5.15 mmol, 85.06% yield, TFA) as a white solid.

### Step 3

To a solution of (2R)-2-amino-N-(2-phenylethyl)butanediamide (200 mg, 572.57 umol, 1 eq, TFA) in H₂SO₄ (0.5 M, 3.40 mL, 2.97 eq) was added NaNO₂ (237.03 mg, 3.44 mmol, 6 eq) in H₂O (0.8 mL) at 0° C. The mixture was stirred at 25° C. for 3 hr. The mixture was concentrated under reduced pressure to give compound (2R)-2-hydroxy-N-(2-phenylethyl)butanediamide (135 mg, crude) as a white solid.

### Step 4

To a solution of (2R)-2-hydroxy-N-(2-phenylethyl)butanediamide (135 mg, 571.39 umol, 1 eq) and octanoyl octanoate (231.76 mg, 857.08 umol, 1.5 eq) in DMF (5 mL) was added DIPEA (221.54 mg, 1.71 mmol, 298.58 uL, 3 eq). The mixture was stirred at 25° C. for 1 hr. The mixture was concentrated under reduced pressure to give a residue. The residue was purified by prep-HPLC(column: Phenomenex Luna C18 100*30 mm*5 um; mobile phase: [water(0.1% TFA)-ACN]; B %: 35%-65%, 10 min) to give compound [(1R)-3-amino-3-oxo-1-(2-phenylethylcarbamoyl)propyl]octanoate (14 mg, 38.24 umol, 6.69% yield, 99% purity) as a white solid.

### Example 187: (R)-N⁴-methyl-2-octanamido-N′-phenethylsuccinamide

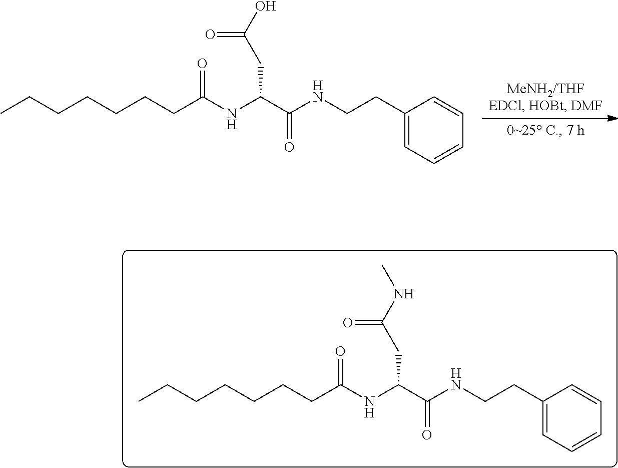 [SMILES: CCCCCCCC(=O)N[C@H](CC(=O)O)C(=O)NCCc1ccccc1 ; CCCCCCCC(=O)N[C@H](CC(=O)NC)C(=O)NCCc1ccccc1]

To a solution of MeNH₂ (2 M, 1.03 mL, 3 eq) in DMF (2.5 mL) was added (3R)-3-(octanoylamino)-4-oxo-4-(2-phenylethylamino)butanoic acid (250 mg, 689.73 umol, 1 eq), 1-hydroxybenzotriazole (121.16 mg, 896.65 umol, 1.3 eq) and 3-(ethyliminomethyleneamino)-N,N-dimethyl-propan-1-amine hydrochloride (171.89 mg, 896.65 umol, 1.3 eq) at 0° C. Then the mixture was allowed to warm to 25° C., and the mixture was stirred for 7 hr. LCMS showed desired mass was detected. The reaction mixture was treated with water (8 mL), and extracted with EtOAc (15 mL*3), washed with brine (40 mL). The combined organic phase was treated with MeOH (50 mL) to get homogeneous solution, dried with anhydrous Na₂SO₄, filtered and concentrated in vacuum. The reaction mixture was treated with EtOAc (50 mL), and the precipitate was filtered and the filter cake was concentrated to give compound (2R)-N′-methyl-2-(octanoylamino)-N-(2-phenylethyl) utanediamide (69 mg, 98.85% purity) as a white solid.

### Example 188: (R)-N⁴,N⁴-dimethyl-2-octanamido-N′-phenethylsuccinamide

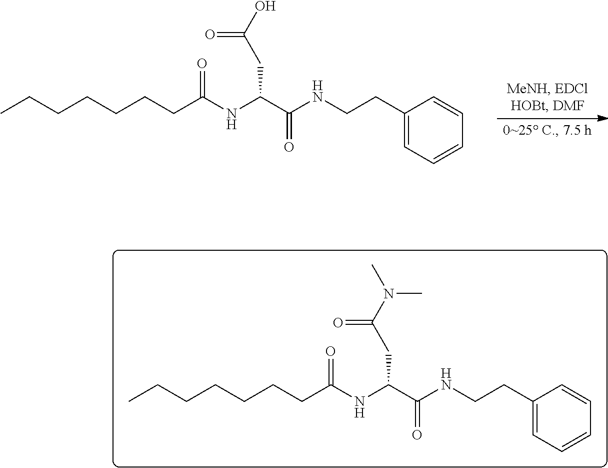 [SMILES: CCCCCCCC(=O)N[C@H](CC(=O)O)C(=O)NCCc1ccccc1 ; CCCCCCCC(=O)N[C@H](CC(=O)N(C)C)C(=O)NCCc1ccccc1]

To a solution of (3R)-3-(octanoylamino)-4-oxo-4-(2-phenylethylamino)butanoic acid (200 mg, 551.78 umol, 1 eq) in DMF (2 mL) was added 3-(ethyliminomethyleneamino)-N,N-dimethyl-propan-1-amine hydrochloride (137.51 mg, 717.32 umol, 1.3 eq) and 1-hydroxybenzotriazole (96.93 mg, 717.32 umol, 1.3 eq) at 0° C. The mixture was stirred at 0° C. for 0.5 hr. Then Me₂NH (2 M, 827.67 uL, 3 eq) was added to the mixture and the mixture was allowed to warm to 25° C. The mixture was further stirred for 7 hr. The reaction mixture was treated with water (5 mL) and extracted with EtOAc (10 mL*4). The combined organic phase was washed with brine (15 mL), dried with anhydrous Na₂SO₄, filtered and concentrated in vacuum. The residue was purified by prep-HPLC (column: Phenomenex Gemini-NX C18 75*30 mm*3 um; mobile phase: [water(10 mM NH₄HCO₃)-ACN]; B %: 35%-60%, 8 min) to give compound(2R)-N′,N′-dimethyl-2-(octanoylamino)-N-(2-phenylethyl)butanediamide (82 mg, 100% purity) as a white solid.

### Example 189: (R)-2-octanamido-N′-(2-(piperidin-4-yl)ethyl)succinamide

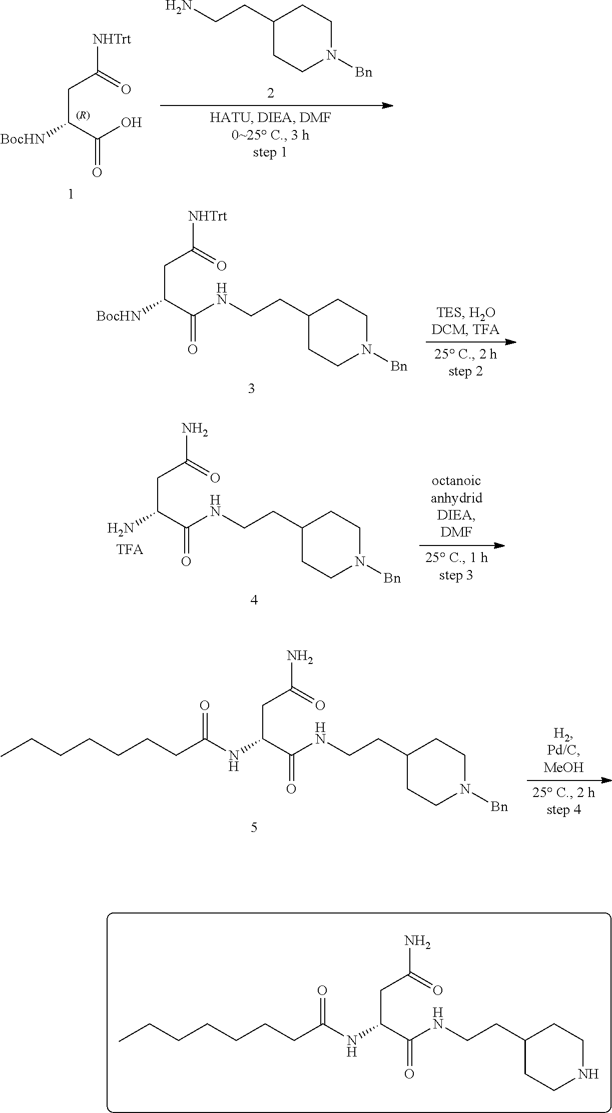

### Step 1

To a solution of (2R)-2-(tert-butoxycarbonylamino)-4-oxo-4-(tritylamino)butanoic acid (800 mg, 1.69 mmol, 1 eq) and 2-(1-benzyl-4-piperidyl)ethanamine (441.69 mg, 2.02 mmol, 1.2 eq) in DMF (10 mL) was added HATU (769.20 mg, 2.02 mmol, 1.2 eq) and DIPEA (479.34 mg, 3.71 mmol, 646.01 uL, 2.2 eq) at 0° C. The mixture was stirred at 25° C. for 3 hr. TLC showed the starting reactant was consumed and one new spot was formed. The reaction mixture was quenched by addition H₂O 30 mL at 0° C. The precipitate was filtered and the filter cake was concentrated under reduced pressure to give compound tert-butyl N-[(1R)-1-[2-(1-benzyl-4-piperidyl) ethylcarbamoyl]-3-oxo-3-(tritylamino)propyl]carbamate (1 g, 1.48 mmol, 87.90% yield) as a white solid.

### Step 2

Put tert-butyl N-[(1R)-1-[2-(1-benzyl-4-piperidyl)ethylcarbamoyl]-3-oxo-3-(tritylamino)propyl]carbamate (800 mg, 1.19 mmol, 1 eq), TES (0.2 mL), H₂O (0.2 mL), DCM (0.4 mL), TFA (7.2 mL). The mixture was stirred at 25° C. for 2 hr. TLC showed the starting reactant was consumed. The mixture was concentrated under reduced pressure to give compound (2R)-2-amino-N-[2-(1-benzyl-4-piperidyl)ethyl]butanediamide (500 mg, crude, TFA) as a white solid.

### Step 3

To a solution of (2R)-2-amino-N-[2-(1-benzyl-4-piperidyl)ethyl]butanediamide (500 mg, 1.12 mmol, 1 eq, TFA) and octanoyl octanoate (454.25 mg, 1.68 mmol, 1.5 eq) in DMF (5 mL) was added DIPEA (434.22 mg, 3.36 mmol, 585.21 uL, 3 eq). The mixture was stirred at 25° C. for 1 hr. The mixture was concentrated under reduced pressure to give a residue. The residue was purified by column chromatography (SiO₂, EtOAc/MeOH=1/0 to 2/1) to give compound (2R)-N-[2-(1-benzyl-4-piperidyl)ethyl]-2-(octanoylamino)butanediamide (420 mg, 915.76 umol, 81.77% yield) as colorless oil.

### Step 4

To a solution of (2R)-N-[2-(1-benzyl-4-piperidyl)ethyl]-2-(octanoylamino)butanediamide (100 mg, 218.04 umol, 1 eq) in MeOH (5 mL) was added Pd/C (0.1 g, 10%). The mixture was stirred at 25° C. for 2 hr under H₂ (15 psi). The mixture was filtered and the filtrate was concentrated to get a residue. The residue was purified by prep-HPLC (column: Phenomenex Luna C18 100*30 mm*5 um; mobile phase: [water(0.04% HCl)-ACN]; B %: 20%-50%, 10 min) to give compound (2R)-2-(octanoylamino)-N-[2-(4-piperidyl)ethyl]butanediamide (10 mg, 24.69 umol, 11.33% yield, HCl) as a white solid.

### Example 190: (R)-N′-(2-(1-methylpiperidin-4-yl)ethyl)-2-octanamidosuccinamide

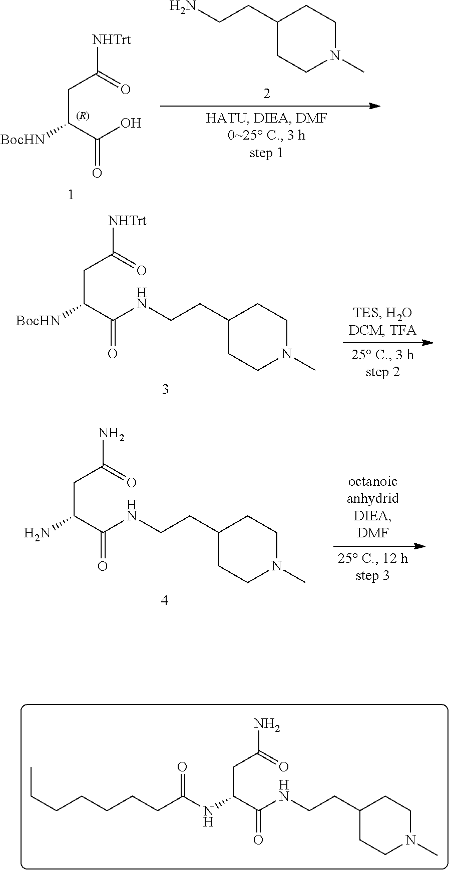

### Step 1

To a mixture of (2R)-2-(tert-butoxycarbonylamino)-4-oxo-4-(tritylamino)butanoic acid (1 g, 2.11 mmol, 1 eq) and 2-(1-methyl-4-piperidyl)ethanamine (359.69 mg, 2.53 mmol, 1.2 eq) in DMF (30 mL) was added HATU (961.50 mg, 2.53 mmol, 1.2 eq) and DIPEA (599.16 mg, 4.64 mmol, 807.49 uL, 2.2 eq) at 0° C. under N₂. The mixture was stirred at 25° C. for 3 hours. LCMS showed the reaction was completed and desired mass was detected. To the mixture was added water (60 mL) and brine (60 mL). The mixture was extracted with ethyl acetate (60 mL*3). The combined organic phase was washed with brine (100 mL), dried with anhydrous Na₂SO₄, filtered and concentrated in vacuum to get tert-butyl N-[(1R)-1-[2-(1-methyl-4-piperidyl)ethylcarbamoyl]-3-oxo-3-(tritylamino)propyl]carbamate (1 g, crude) as a yellow oil.

### Step 2

A mixture of tert-butyl N-[(1R)-1-[2-(1-methyl-4-piperidyl)ethylcarbamoyl]-3-oxo-3-(tritylamino) propyl]carbamate (1 g, 1.67 mmol, 1 eq), TFA (30.80 g, 269.98 mmol, 20.00 mL, 156.06 eq), DCM (2.64 g, 31.08 mmol, 2 mL, 8.98 eq), H₂O (1.00 g, 55.49 mmol, 1 mL, 16.04 eq), and triethylsilane (728.00 mg, 6.26 mmol, 1 mL, 1 eq) was stirred at 25° C. for 3 h. The mixture was concentrated in vacuum to get (2R)-2-amino-N-[2-(1-methyl-4-piperidyl)ethyl]butanediamide (500 mg, crude, TFA) as a white solid.

### Step 3

To a mixture of (2R)-2-amino-N-[2-(1-methyl-4-piperidyl)ethyl]butanediamide (500 mg, 1.35 mmol, 1 eq, TFA) and octanoyl octanoate (547.58 mg, 2.03 mmol, 1.5 eq) in DMF (15 mL) was added DIPEA (872.38 mg, 6.75 mmol, 1.18 mL, 5 eq) under N2. The mixture was stirred at 25° C. for 3 hour. LCMS showed the reaction was completed and desired mass was detected. To the mixture was added water (30 mL). The mixture was extracted with ethyl acetate (20 mL*3). The combined organic phase was washed with brine (30 mL), dried with anhydrous Na₂SO₄, filtered and concentrated in vacuum. The residue was purified by p-HPLC (column: Phenomenex Gemini-NX C18 75*30 mm*3 um; mobile phase: [water(10 mM NH₄HCO₃)-ACN]; B %: 5%-35%, 12 min) to give (2R)-N-[2-(1-methyl-4-piperidyl)ethyl]-2-(octanoylamino)butanediamide (150 mg, 392.12 umol, 29.05% yield, 100% purity) as a white solid.

### Example 191: (R)-2-octanamido-N′-(2-(piperazin-1-yl)ethyl)succinamide

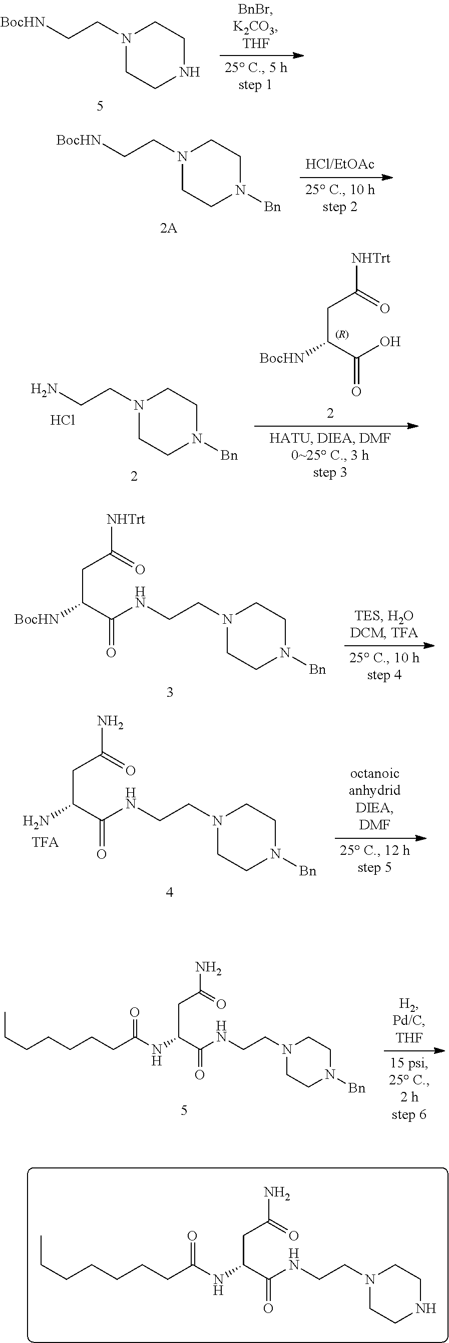

### Step 1

To a solution of tert-butyl N-(2-piperazin-1-ylethyl)carbamate (1 g, 4.36 mmol, 1 eq) in THF (10 mL) was added K₂CO₃ (1.21 g, 8.72 mmol, 2 eq) and bromomethylbenzene (895.00 mg, 5.23 mmol, 621.53 uL, 1.2 eq) was degassed and purged with N₂ for 3 times. The mixture was stirred at 25° C. for 5 hr under N2 atmosphere. The combined organic phase was diluted with EtOAc 100 mL and washed with water 120 mL (40 mL*3) and brine 40 mL (20 mL*2), dried with anhydrous Na₂SO₄, filtered and concentrated under reduced pressure to give a residue. The residue was purified by column chromatography (SiO₂, Petroleum ether/Ethyl acetate=1/0 to 3/1) to give compound tert-butyl N-[2-(4-benzylpiperazin-1-yl)ethyl]carbamate (400 mg, 1.18 mmol, 26.99% yield, 94% purity) as white solid.

### Step 2

A solution of tert-butyl N-[2-(4-benzylpiperazin-1-yl)ethyl]carbamate (400 mg, 1.25 mmol, 1 eq) in HCV/EtOAc (4 M, 2.87 mL, 9.17 eq) was stirred at 25° C. for 10 hr under N₂ atmosphere. The reaction mixture was concentrated under reduced pressure to give compound 2-(4-benzylpiperazin-1-yl)ethanamine (200 mg, 781.90 umol, 62.44% yield, HCl) as white solid.

### Step 3

A mixture of (2R)-2-(tert-butoxycarbonylamino)-4-oxo-4-(tritylamino)butanoic acid (247.37 mg, 521.27 umol, 1 eq) and 2-(4-benzylpiperazin-1-yl)ethanamine (200 mg, 781.90 umol, 265.81 uL, 1.5 eq, HCl) in DMF (10 mL) was added HATU (237.84 mg, 625.52 umol, 1.2 eq) and DIEA (202.11 mg, 1.56 mmol, 272.38 uL, 3 eq) at 0° C. The mixture was stirred at 25° C. for 3 hr. The reaction mixture was quenched by addition H₂O 60 mL at 0° C. The precipitate was filtered and filter cake was concentrated under reduced pressure to give compound tert-butyl N-[(1R)-1-[2-(4-benzylpiperazin-1-yl)ethylcarbamoyl]-3-oxo-3-(tritylamino)propyl]carbamate (320 mg, 440.33 umol, 84.47% yield, 93% purity) as white solid.

### Step 4

A mixture of tert-butyl N-[(1R)-1-[2-(4-benzylpiperazin-1-yl)ethylcarbamoyl]-3-oxo-3-(tritylamino)propyl]carbamate (270 mg, 399.49 umol, 1 eq), TES (228.96 mg, 998.73 umol, 0.25 mL, 2.5 eq), H₂O (250.00 mg, 13.87 mmol, 250.00 uL, 34.73 eq), DCM (660.00 mg, 7.77 mmol, 500.00 uL, 19.45 eq) and TFA (13.86 g, 121.56 mmol, 9.00 mL, 304.28 eq) was stirred at 25° C. for 10 hr under N₂ atmosphere. The reaction mixture was concentrated under reduced pressure to give compound (2R)-2-amino-N-[2-(4-benzylpiperazin-1-yl)ethyl]butanediamide (150 mg, crude) as white solid.

### Step 5

A solution of (2R)-2-amino-N-[2-(4-benzylpiperazin-1-yl)ethyl]butanediamide (150 mg, 335.23 umol, 1 eq, TFA) in DMF (5 mL) was added DIEA (129.98 mg, 1.01 mmol, 175.17 uL, 3 eq), octanoyl octanoate (135.97 mg, 502.85 umol, 1.5 eq) and was degassed and purged with N2 for 3 times. The mixture was stirred at 25° C. for 12 hr under N2 atmosphere. TLC showed desired spot was detected. The combined organic phase was diluted with EtOAc 20 mL and washed with water 60 mL (20 mL*3) and brine 40 mL (20 mL*2), dried with anhydrous Na₂SO₄, filtered and the filtrate was concentrated under reduced pressure to give a residue. The residue was purified by prep-TLC (SiO₂, Ethyl acetate: MeOH=1:1) to give compound (2R)-N-[2-(4-benzylpiperazin-1-yl)ethyl]-2-(octanoylamino)butanediamide (100 mg, 217.57 umol, 64.90% yield) as white solid.

### Step 6

To a solution of methyl (2R)-N-[2-(4-benzylpiperazin-1-yl)ethyl]-2-(octanoylamino) butanediamide (0.1 g, 217.57 umol, 1 eq) in THF (3 mL) was added Pd/C (0.01 g, 217.57 umol, 10% purity, 1 eq) under N₂. The suspension was degassed under vacuum and purged with H₂ several times. The mixture was stirred under H₂ (15 psi) at 25° C. for 2 hr. The reaction mixture was filtered and the filtrate was concentrated under reduced pressure to give a residue. The residue was purified by prep-HPLC (column: Phenomenex Gemini-NX C18 75*30 mm*3 um; mobile phase: [water(10 mM NH4HCO3)-ACN]; B %: 15%-30%, 12 min) to give compound (2R)-2-(octanoylamino)-N-(2-piperazin-1-ylethyl)butanediamide (8.5 mg, 22.08 umol, 10.15% yield, 96% purity) as white solid.

### Example 192: (R)-2-decanamido-N′-(4-hydroxyphenethyl)succinamide

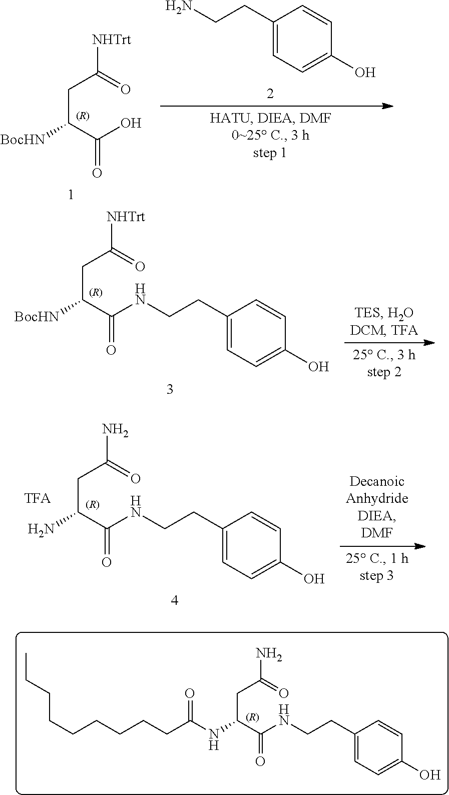

### Step 1

To a mixture of (2R)-2-(tert-butoxycarbonylamino)-4-oxo-4-(tritylamino)butanoic acid (2 g, 4.21 mmol, 1 eq) and 4-(2-aminoethyl)phenol (693.77 mg, 5.06 mmol, 1.2 eq) in DMF (60 mL) was added HATU (1.92 g, 5.06 mmol, 1.2 eq) and DIPEA (1.20 g, 9.27 mmol, 1.61 mL, 2.2 eq) at 0° C. under N₂. The mixture was stirred at 25° C. for 3 hours. LCMS showed the reaction was completed and desired mass was detected. The mixture was cooled to 0° C. and water (120 mL) was added slowly. Then the precipitate was filtered and the filter cake was washed by water (50 mL*2). The filter cake was concentrated in vacuum to get tert-butyl N-[(1R)-1-[2-(4-hydroxyphenyl)ethylcarbamoyl]-3-oxo-3-(tritylamino)propyl]carbamate (2.2 g, 3.56 mmol, 84.41% yield, 96% purity) as a white solid.

### Step 2

A mixture of tert-butyl N-[(1R)-1-[2-(4-hydroxyphenyl)ethylcarbamoyl]-3-oxo-3-(tritylamino) propyl]carbamate (2 g, 3.37 mmol, 1 eq), TFA (55.44 g, 486.23 mmol, 36.00 mL, 144.34 eq), DCM (5.28 g, 62.17 mmol, 4 mL, 18.46 eq), H₂O (2.00 g, 110.99 mmol, 2 mL, 32.95 eq) and triethylsilane (1.46 g, 12.52 mmol, 2 mL, 3.72 eq) was stirred at 25° C. for 3 h. LCMS showed the reaction was completed and desired mass was detected. The mixture was concentrated in vacuum get the crude compound as TFA salt. Then the crude compound was treated by sat. NaHCO₃ (100 mL) to pH=8. The aqueous phase was extracted with ethyl acetate (50 mL*2). Then the aqueous phase was concentrated in vacuum. The residue was purified by silica gel chromatography (silica gel, Petroleum ether/Ethyl acetate=1/1, 0/1 to THF/MeOH=1/0, 3/1) to get (2R)-2-amino-N-[2-(4-hydroxyphenyl)ethyl]butanediamide (250 mg, 835.72 umol, 24.81% yield, 84% purity) as a yellow oil.

### Step 3

To a mixture of (2R)-2-amino-N-[2-(4-hydroxyphenyl)ethyl]butanediamide (300 mg, 509.16 umol, 62% purity, 1 eq, TFA) and decanoyl decanoate (199.50 mg, 611.00 umol, 1.2 eq) in DMF (10 mL) was added DIPEA (329.02 mg, 2.55 mmol, 443.43 uL, 5 eq) under N2. The mixture was stirred at 25° C. for 1 hour. LCMS showed the reaction was completed and desired mass was detected. To the mixture was added water (30 mL) at 0° C. Then the mixture was filtered and the filter cake was washed by water (20 mL*2) and concentrated in vacuum. The residue was purified by prep-HPLC (column: Phenomenex Gemini-NX C18 75*30 mm*3 um; mobile phase: [water(10 mM NH4HCO3)-ACN]; B %: 25%-55%, 10 min) to get (2R)-2-(decanoylamino)-N-[2-(4-hydroxyphenyl)ethyl]butanediamide (49 mg, 116.00 umol, 22.78% yield, 96% purity) as a white solid.

### Example 193: (R)-2-(4-cyclohexylbutanamido)-N′-(4-hydroxyphenethyl)succinamide

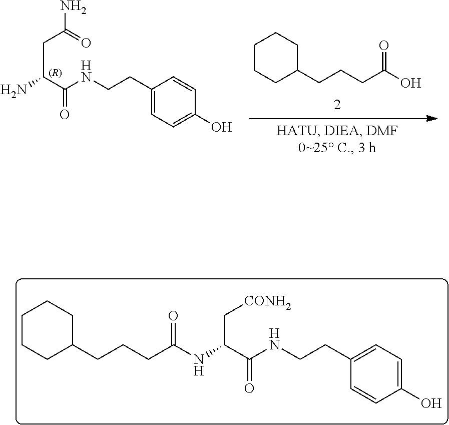

To a mixture of (2R)-2-amino-N-[2-(4-hydroxyphenyl)ethyl]butanediamide (200 mg, 795.92 umol, 1 eq) and 4-cyclohexylbutanoic acid (162.60 mg, 955.11 umol, 1.2 eq) in DMF (4 mL) was added HATU (363.16 mg, 955.11 umol, 1.2 eq) and DIPEA (205.73 mg, 1.59 mmol, 277.26 uL, 2 eq) at 0° C. under N2. The mixture was stirred at 25° C. for 3 hour. LCMS showed the reaction was completed and desired mass was detected. The mixture was concentrated in vacuum. The residue was purified by prep-HPLC (column: Phenomenex Gemini-NX C18 75*30 mm*3 um; mobile phase: [water(10 mM NH4HCO3)-ACN]; B %: 20%-50%, 10 min) to give (2R)-2-(4-cyclohexylbutanoyl amino)-N-[2-(4-hydroxyphenyl)ethyl]butanediamide (81 mg, 98% purity, 100% ee) as a white solid.

### Example 194: (R)-N′-(3-hydroxyphenethyl)-2-octanamidosuccinamide

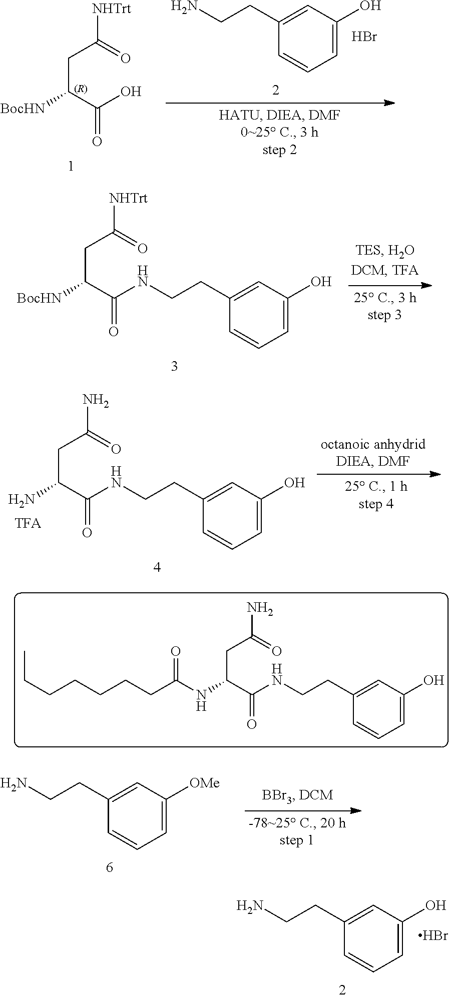

### Step 1

To a stirred solution of 2-(3-methoxyphenyl)ethanamine (1 g, 6.61 mmol, 970.87 uL, 1 eq) in DCM (30 mL) was added BBr₃ (4.97 g, 19.84 mmol, 3 eq) in DCM (7.5 mL) drop wise at −78° C. under N₂ atmosphere. The mixture was allowed to slowly warm to 25° C. and then the reaction mixture was stirred at 25° C. for 20 hr. LCMS showed the reaction was completed. The reaction mixture was treated with DCM (50 mL), and then extracted with water (60 mL*3), and the combined aqueous phases were concentrated in vacuum to get compound 3-(2-aminoethyl) phenol (1.5 g, crude, HBr) as a yellow solid.

### Step 2

To a solution of (2R)-2-(tert-butoxycarbonylamino)-4-oxo-4-(tritylamino)butanoic acid (900 mg, 1.90 mmol, 1 eq) and 3-(2-aminoethyl)phenol (390.25 mg, 2.84 mmol, 1.5 eq) in DMF (15 mL) was added HATU (721.12 mg, 1.90 mmol, 1 eq) and DIPEA (245.12 mg, 1.90 mmol, 330.34 uL, 1 eq) at 0° C., and then the mixture was stirred at 25° C. for 3 hr. The desired mass was detected by LCMS and the reaction was completed. The reaction mixture was cooled to 0° C. Then the mixture was treated with water (25 mL), and the precipitate was filtered and the filter cake was concentrated in vacuum to give the crude product as a white solid. The crude product was purified by prep-TLC to give compound tert-butyl N-[(1R)-1-[2-(3-hydroxyphenyl) ethylcarbamoyl]-3-oxo-3-(tritylamino) propyl]carbamate as a white solid.

### Step 3

The tert-butyl N-[(1R)-1-[2-(3-hydroxyphenyl)ethylcarbamoyl]-3-oxo-3-(tritylamino)propyl]carbamate (1 g, 1.68 mmol, 1 eq), TES (386.13 mg, 1.68 mmol, 0.75 mL, 1 eq), H₂O (625.00 mg, 34.69 mmol, 625.00 uL, 20.60 eq), TFA (34.65 g, 303.89 mmol, 22.50 mL, 180.42 eq) and DCM (1.65 g, 19.43 mmol, 1.25 mL, 11.53 eq) was stirred at 25° C. for 3 hr. The desired mass was detected by LCMS and the reaction was completed. The reaction mixture was concentrated to give compound (2R)-2-amino-N-[2-(3-hydroxyphenyl) ethyl]butanediamide (1.05 g, crude, TFA) as a yellow solid.

### Step 4

To a solution of (2R)-2-amino-N-[2-(3-hydroxyphenyl)ethyl]butanediamide (50 mg, 30.45 umol, 22.25% purity, 1 eq, TFA) and octanoyl octanoate (12.35 mg, 45.68 umol, 1.5 eq) in DMF (1 mL) was added DIPEA (11.81 mg, 91.36 umol, 15.91 uL, 3 eq). The mixture was stirred at 25° C. for 1 hr. The reaction mixture was concentrated in vacuum to get a residue which was purified by prep-HPLC (column: Phenomenex luna C18 250*50 mm*10 um; mobile phase: [water(0.04% HCl)-ACN]; B %: 30%-60%, 10 min) to give compound (2R)-N-[2-(3-hydroxyphenyl) ethyl]-2-(octanoylamino)butanediamide (54 mg, 97.89% purity) as a white solid.

### Example 195: (R)-N′-(2-hydroxyphenethyl)-2-octanamidosuccinamide

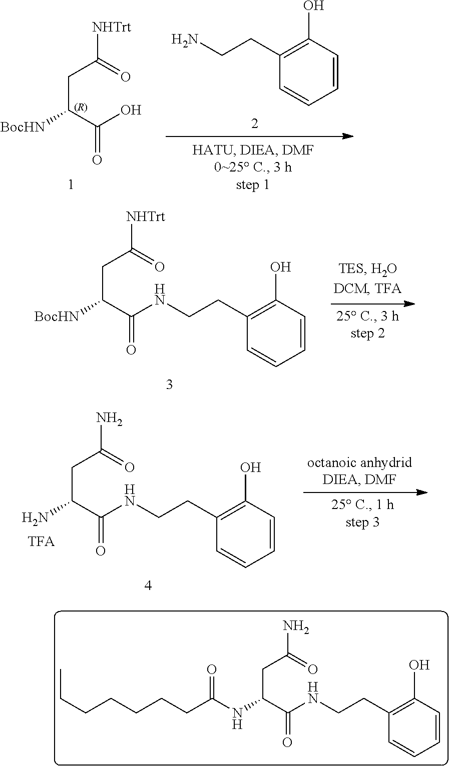

### Step 1

To a solution of (2R)-2-(tert-butoxycarbonylamino)-4-oxo-4-(tritylamino)butanoic acid (1 g, 2.11 mmol, 1 eq) and 2-(2-aminoethyl)phenol (346.89 mg, 2.53 mmol, 1.2 eq) in DMF (15 mL) was added HATU (801.25 mg, 2.11 mmol, 1 eq) and DIPEA (272.35 mg, 2.11 mmol, 367.05 uL, 1 eq) at 0° C., and then the mixture was stirred at 25° C. for 3 hr. The reaction mixture was cooled to 0° C., treated with H₂O 25 mL, and the precipitate was filtered and the filter cake was concentrated to give compound tert-butyl N-[(1R)-1-[2-(2-hydroxyphenyl)ethylcarbamoyl]-3-oxo-3-(tritylamino)propyl]carbamate (1.1 g, 1.41 mmol, 66.82% yield, 76% purity) as a white solid.

### Step 2

The tert-butyl N-[(1R)-1-[2-(2-hydroxyphenyl)ethylcarbamoyl]-3-oxo-3-(tritylamino) propyl]carbamate (1.1 g, 1.85 mmol, 1 eq) in TES (424.75 mg, 1.85 mmol, 0.75 mL, 1 eq), H₂O (670.73 mg, 37.23 mmol, 670.73 uL, 20.10 eq), DCM (1.77 g, 20.85 mmol, 1.34 mL, 11.25 eq) and TFA (37.19 g, 326.12 mmol, 24.15 mL, 176.02 eq) was stirred at 25° C. for 3 hr. Desired mass was detected by LCMS. The reaction was concentrated in vacuum to give compound (2R)-2-amino-N-[2-(2-hydroxyphenyl)ethyl]butanediamide (1.05 g, crude, TFA) as a yellow solid.

### Step 3

To a solution of (2R)-2-amino-N-[2-(2-hydroxyphenyl)ethyl]butanediamide (1.05 g, 574.86 umol, 20% purity, 1 eq, TFA) and octanoyl octanoate (108.81 mg, 402.40 umol, 0.7 eq) in DMF (10 mL) was added DIPEA (222.89 mg, 1.72 mmol, 300.39 uL, 3 eq). The mixture was stirred at 25° C. for 1 hr. Desired mass was detected by LCMS. The reaction mixture was concentrated in vacuum. The reaction mixture was purified by prep-HPLC (column: Kromasil C18 (250*50 mm*10 um); mobile phase: [water(10 mM NH₄HCO₃)-ACN]; B %: 25%-55%, 10 min) to give compound (2R)-N-[2-(2-hydroxyphenyl) ethyl]-2-(octanoylamino) butanediamide (70 mg, 174.26 umol, 35.37% yield, 93.97% purity) as a white solid.

### Example 196: (R)-N′-(2,4-dihydroxyphenethyl)-2-octanamidosuccinamide

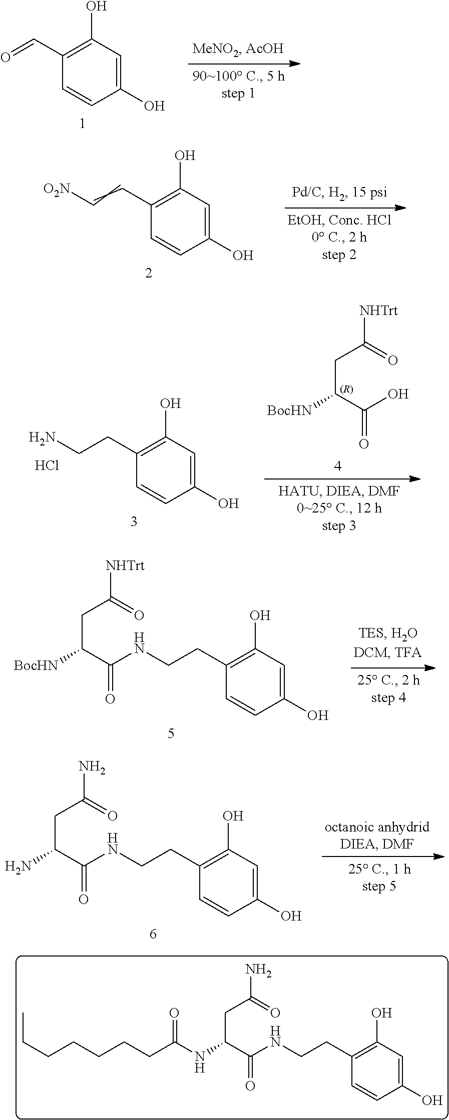

### Step 1

To a solution of NH₄OAc (3.49 g, 45.25 mmol, 2.5 eq), AcOH (70 mL) and nitromethane (39.55 g, 647.94 mmol, 35.00 mL, 35.80 eq) was added 2,4-dihydroxybenzaldehyde (2.5 g, 18.10 mmol, 1 eq) at 90° C., then the mixture was stirred at 100° C. for 5 h. LCMS showed the starting reactant consumed. The reaction mixture was cooled to 15° C., quenched with H₂O (200 mL) and sat·NaHCO₃ (500 mL), then extracted with ethyl acetate (100 mL*3). The organic extracts was dried with Na₂SO₄ and concentrated in vacuo. The residue was purified by column chromatography. (SiO₂, Petroleum ether/Ethyl acetate=10/1 to 1/1) to give the product 4-[(E)-2-nitrovinyl]benzene-1,3-diol (1.3 g, 8.28 mmol, 45.75% yield) as brown oil.

### Step 2

To a solution of Pd/C (200 mg, 10% purity) and HCl (12 M, 92.01 uL, 1 eq) in EtOH (10 mL) was added 4-[(E)-2-nitrovinyl]benzene-1,3-diol (200 mg, 1.10 mmol, 1 eq) at 0° C. The mixture was stirred at 0° C. for 2 h under H₂ under 15 psi. LCMS showed the starting reactant consumed. The mixture was filtered and the filtrate was concentrated under reduced pressure to give the product 4-(2-aminoethyl)benzene-1,3-diol (167 mg, crude, HCl) as a brown solid.

### Step 3

To a solution of (2R)-2-(tert-butoxycarbonylamino)-4-oxo-4-(tritylamino)butanoic acid (250 mg, 526.82 umol, 1 eq) and 4-(2-aminoethyl)benzene-1,3-diol (149.86 mg, 790.23 umol, 1.5 eq, HCl) in DMF (5 mL) was added HATU (240.37 mg, 632.18 umol, 1.2 eq) and DIPEA (204.26 mg, 1.58 mmol, 275.28 uL, 3 eq) at 0° C. The mixture was stirred at 25° C. for 12 h. TLC showed the starting reactant consumed. The mixture was poured into H₂O (10 mL), then filtered and the filter cake was concentrated to give the product tert-butyl N-[(1R)-1-[2-(2,4-dihydroxyphenyl) ethyl carbamoyl]-3-oxo-3-(tritylamino)propyl]carbamate (200 mg, crude) as a brown solid.

### Step 4

The tert-butyl N-[(1R)-1-[2-(2,4-dihydroxyphenyl)ethylcarbamoyl]-3-oxo-3-(tritylamino) propyl]carbamate (200 mg, 328.02 umol, 1 eq) was dissolved with TES (0.5 mL), H₂O (0.5 mL), DCM (1 mL) and TFA (18 mL). The mixture was stirred at 25° C. for 2 h. LCMS showed the starting reactant consumed. The mixture was concentrated under reduced pressure to give the product (2R)-2-amino-N-[2-(2,4-dihydroxyphenyl) ethyl]butanediamide (100 mg, crude, TFA) as a brown solid.

### Step 5

To a solution of octanoyl octanoate (106.37 mg, 393.39 umol, 1.5 eq) and (2R)-2-amino-N-[2-(2,4-dihydroxyphenyl)ethyl]butanediamide (100 mg, 262.26 umol, 1 eq, TFA) in DMF (3 mL) was added DIPEA (101.68 mg, 786.78 umol, 137.04 uL, 3 eq). The mixture was stirred at 25° C. for 1 h. LCMS showed the starting reactant was consumed and have the desired mass. The mixture was concentrated under reduced pressure to give a residue. The residue was purified by prep-HPLC (column: Phenomenex Gemini-NX C18 75*30 mm*3 um; mobile phase: [water(0.05% NH3H2O+10 mM NH4HCO3)-ACN]; B %: 20%-40%, 8 min) to give the compound (2R)-2-(octanoylamino)-N-(2-pyrazol-1-ylethyl)butanediamide (30 mg, 81.09 umol, 30.92% yield, 95% purity) as a white solid.

### Example 197: (R)-N′-(3,4-dihydroxyphenethyl)-2-octanamidosuccinamide

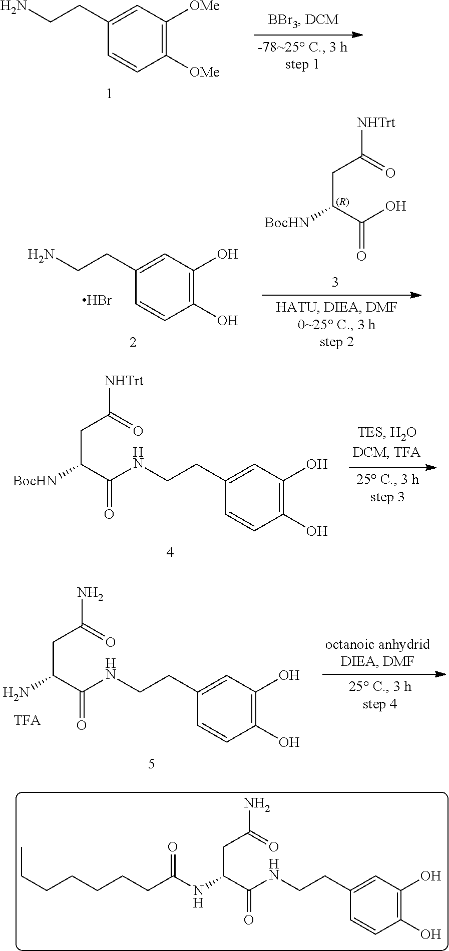

### Step 1

To a mixture of 2-(3,4-dimethoxyphenyl)ethanamine (2.00 g, 11.04 mmol, 1.83 mL, 1 eq) in DCM (20 mL) was added BBr₃ (8.29 g, 33.11 mmol, 3.19 mL, 3 eq) dropwise at −78° C. under N2. The mixture was stirred at 25° C. for 12 hours. LCMS showed the reaction was completed and desired mass was detected. The mixture was quenched by water (30 mL) at −78° C. Then the aqueous phase was extracted with DCM (20 mL*2). The aqueous phase was filtered and concentrated in vacuum to get 4-(2-aminoethyl)benzene-1,2-diol (3 g, crude, HBr) as a brown solid.

### Step 2

To a mixture of (2R)-2-(tert-butoxycarbonylamino)-4-oxo-4-(tritylamino)butanoic acid (1 g, 2.11 mmol, 1 eq) and 4-(2-aminoethyl)benzene-1,2-diol (840.84 mg, 3.16 mmol, 88% purity, 1.5 eq, HBr) in DMF (30 mL) was added HATU (961.50 mg, 2.53 mmol, 1.2 eq) and DIPEA (2.18 g, 16.86 mmol, 2.94 mL, 8 eq) at 0° C. under N₂. The mixture was stirred at 25° C. for 3 hours. The mixture was cooled to 0° C. and water (60 mL) was added slowly. Then the precipitate was filtered and the filter cake was washed by water (30 mL*2). The filter cake was concentrated in vacuum to get tert-butyl N-[(1R)-1-[2-(3,4-dihydroxyphenyl)ethylcarbamoyl]-3-oxo-3-(tritylamino)propyl]carbamate (1 g, 1.31 mmol, 62.27% yield, 80% purity) as a white solid.

### Step 3

A mixture of tert-butyl N-[(1R)-1-[2-(3,4-dihydroxyphenyl)ethylcarbamoyl]-3-oxo-3-(tritylamino) propyl]carbamate (0.9 g, 1.18 mmol, 80% purity, 1 eq), TFA (24.64 g, 216.10 mmol, 16 mL, 183.00 eq), DCM (2.38 g, 27.98 mmol, 1.8 mL, 23.69 eq), H₂O (900.00 mg, 49.94 mmol, 0.9 mL, 42.29 eq) and triethylsilane (655.20 mg, 5.63 mmol, 0.9 mL, 4.77 eq) was stirred at 25° C. for 3 h. LCMS showed the reaction was completed and desired mass was detected. The mixture was concentrated in vacuum to get (2R)-2-amino-N-[2-(3,4-dihydroxyphenyl)ethyl]butanediamide (1 g, crude) as a yellow solid.

### Step 4

To a mixture of (2R)-2-amino-N-[2-(3,4-dihydroxyphenyl)ethyl]butanediamide (1.5 g, 1.46 mmol, 37% purity, 1 eq, TFA) and octanoyl octanoate (432.94 mg, 1.60 mmol, 1.1 eq) in DMF (20 mL) was added DIPEA (1.32 g, 10.19 mmol, 1.77 mL, 7 eq) under N2. The mixture was stirred at 25° C. for 3 hour. To the mixture was added water (50 mL) at 0° C. and the mixture was filtered, the filter cake was concentrated in vacuum to get the crude product. The crude product was purified by p-HPLC (column: Phenomenex luna C18 80*40 mm*3 um; mobile phase: [water(0.04% HCl)-ACN]; B %: 32%-47%, 5.5 min) to give (2R)-N-[2-(3,4-dihydroxyphenyl) ethyl]-2-(octanoylamino)butanediamide (32 mg, 76.92 umol, 5.28% yield, 94.58% purity) as a light pink solid.

### Example 198: (R)-N′-(2-(1H-imidazol-4-yl)ethyl)-2-octanamidosuccinamide

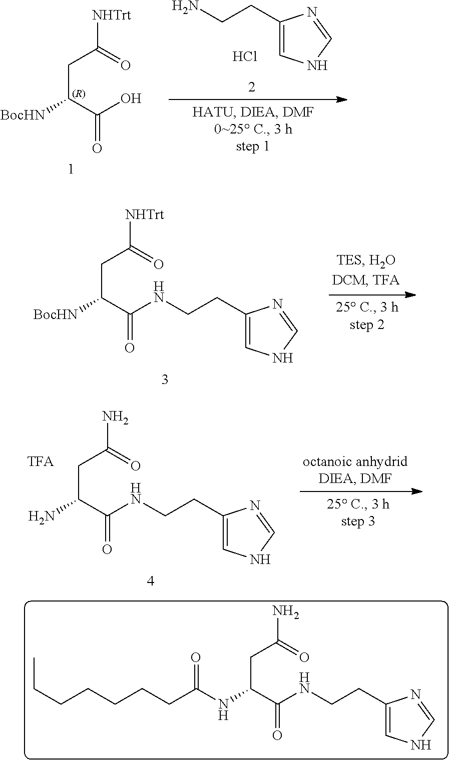

### Step 1

To a mixture of (2R)-2-(tert-butoxycarbonylamino)-4-oxo-4-(tritylamino)butanoic acid (1 g, 2.11 mmol, 1 eq) and 2-(1H-imidazol-4-yl)ethanamine (373.25 mg, 2.53 mmol, 1.2 eq, HCl) in DMF (30 mL) was added HATU (961.50 mg, 2.53 mmol, 1.2 eq) and DIPEA (1.36 g, 10.54 mmol, 1.84 mL, 5 eq) at 0° C. under N2. The mixture was stirred at 25° C. for 3 hours. LCMS showed the reaction was completed and desired mass was detected. The mixture was cooled to 0° C. and water (60 mL) was added slowly, then the precipitate was filtered and the filter cake was washed by water (30 mL*2). The filter cake was concentrated in vacuum to get tert-butyl N-[(1R)-1-[2-(1H-imidazol-4-yl)ethylcarbamoyl]-3-oxo-3-(tritylamino)propyl]carbamate (1 g, 1.39 mmol, 66.04% yield, 79% purity) as a white solid.

### Step 2

A mixture of tert-butyl N-[(1R)-1-[2-(1H-imidazol-4-yl)ethylcarbamoyl]-3-oxo-3-(tritylamino) propyl]carbamate (1 g, 1.39 mmol, 79% purity, 1 eq) in TFA (27.72 g, 243.12 mmol, 18 mL, 174.70 eq), DCM (2.64 g, 31.08 mmol, 2 mL, 22.34 eq), H₂O (1.00 g, 55.49 mmol, 1 mL, 39.88 eq), triethylsilane (728.00 mg, 6.26 mmol, 1 mL, 4.50 eq) was stirred at 25° C. for 3 h. LCMS showed the reaction was completed and desired mass was detected. The mixture was concentrated in vacuum to get (2R)-2-amino-N-[2-(1H-imidazol-4-yl)ethyl]butanediamide (1.3 g, crude, TFA) as a white solid.

### Step 3

To a mixture of (2R)-2-amino-N-[2-(1H-imidazol-4-yl)ethyl]butanediamide (1.2 g, 1.63 mmol, 46% purity, 1 eq, TFA) and octanoyl octanoate (527.95 mg, 1.95 mmol, 1.2 eq) in DMF (30 mL) was added DIPEA (1.47 g, 11.39 mmol, 1.98 mL, 7 eq) under N₂. The mixture was stirred at 25° C. for 3 hour. LCMS showed the reaction was completed and desired mass was detected. To the mixture was added water (50 mL) at 0° C. The precipitate was filtered and the filter cake was washed with water (20 mL*2) and concentrated in vacuum. The residue was purified by p-HPLC (column: Phenomenex luna C18 80*40 mm*3 um; mobile phase: [water(0.04% HCl)-ACN]; B %: 5%-30%, 7 min) to get (2R)-N-[2-(1H-imidazol-4-yl)ethyl]-2-(octanoylamino)butanediamide (55 mg, 99% purity, 100% ee, HCl salt) as a white solid.

### Example 199: (R)-N′-(2-(1H-pyrazol-1-yl)ethyl)-2-octanamidosuccinamide

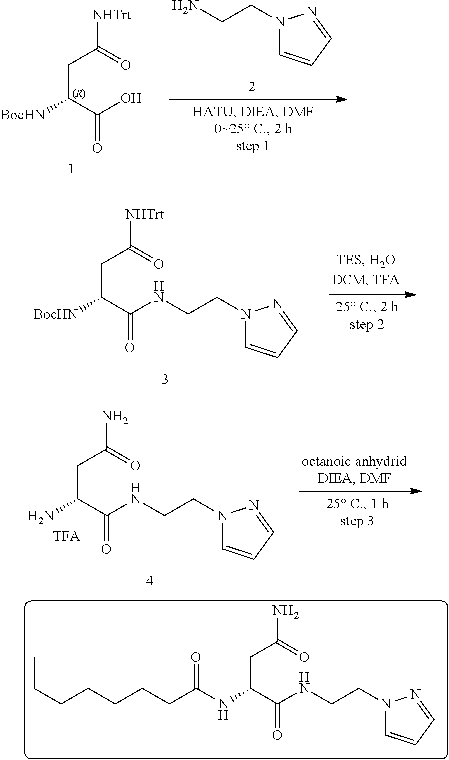

### Step 1

A mixture of (2R)-2-(tert-butoxycarbonylamino)-4-oxo-4-(tritylamino)butanoic acid (1 g, 2.11 mmol, 1 eq), 2-(1H-pyrazol-2-yl)ethanamine (281.06 mg, 2.53 mmol, 1.2 eq), HATU (961.50 mg, 2.53 mmol, 1.2 eq), DIEA (599.16 mg, 4.64 mmol, 807.49 uL, 2.2 eq) in DMF (3 mL) was stirred at 0° C. for 0.5 h, then stirred at 25° C. for 1.5 h under N₂ atmosphere. The reaction mixture was quenched by addition H₂O 100 mL at 0° C., and extracted with EtOAc 90 mL (30 mL*3). The combined organic layers were washed with brine 100 mL, dried over Na₂SO₄, filtered and the filtrate was concentrated under reduced pressure to give a residue to give compound tert-butyl N-[(1R)-3-oxo-1-[2-(1H-pyrazol-2-yl)ethylcarbamoyl]-3-(tritylamino) propyl]carbamate (1 g, 80.72% yield) as a white solid.

### Step 2

The tert-butyl N-[(1R)-3-oxo-1-(2-pyrazol-1-ylethylcarbamoyl)-3-(tritylamino)propyl]carbamate (300 mg, 528.47 umol, 1 eq) was dissolved with TES (0.5 mL), H₂O (0.5 mL), DCM (1 mL) and TFA (18 mL), then the reaction mixture was stirred at 25° C. for 2 h. TLC showed the starting reactant was consumed. The mixture was concentrated under reduced pressure to give the crude product (2R)-2-amino-N-(2-pyrazol-1-ylethyl)butanediamide (180 mg, crude, TFA) as a white solid.

### Step 3

To a solution of (2R)-2-amino-N-(2-pyrazol-1-ylethyl)butanediamide (180 mg, 530.55 umol, 1 eq, TFA) and octanoyl octanoate (215.20 mg, 795.83 umol, 1.5 eq) in DMF (3 mL) was added DIPEA (205.71 mg, 1.59 mmol, 277.24 uL, 3 eq) at 25° C. The mixture was stirred at 25° C. for 1 h. The mixture was filtered and the filter cake was concentrated under reduced pressure to give a residue, then purified by re-crystallization with EtOAc(5 mL) at 25° C. to get the compound (2R)-2-(octanoylamino)-N-(2-pyrazol-1-ylethyl)butanediamide (110 mg, 313.00 umol, 58.99% yield) as a white solid.

### Example 200: (R)-N′-(2-(1H-imidazol-1-yl)ethyl)-2-octanamidosuccinamide

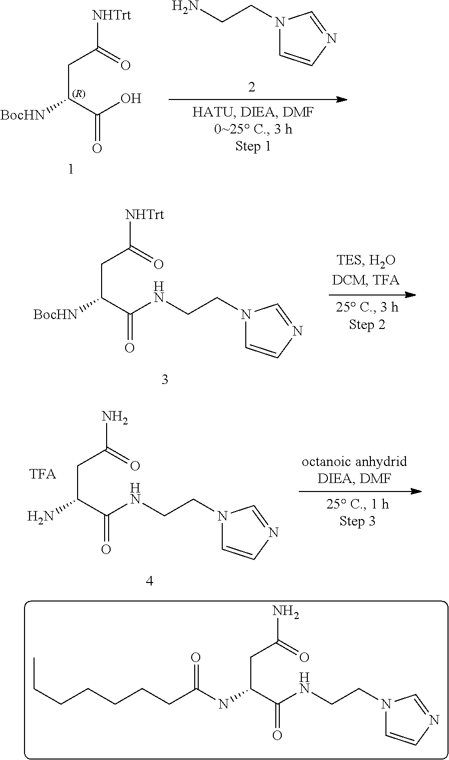

### Step 1

To a solution of (2R)-2-(tert-butoxycarbonylamino)-4-oxo-4-(tritylamino)butanoic acid (1 g, 2.11 mmol, 1 eq) and 2-imidazol-1-ylethanamine (281.05 mg, 2.53 mmol, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 3 hr. The reaction mixture was poured to water, filtered and the filter cake was concentrated under reduced pressure to give the product tert-butyl N-[(1R)-1-(2-imidazol-1-ylethylcarbamoyl)-3-oxo-3-(tritylamino)propyl]carbamate (1 g, 1.57 mmol, 74.40% yield, 89% purity) as white solid.

### Step 2

A mixture of TFA (27.72 g, 243.11 mmol, 18.00 mL, 138.01 eq), DCM (1.32 g, 15.54 mmol, 1.00 mL, 8.82 eq), H₂O (500.00 mg, 27.75 mmol, 0.5 mL, 15.76 eq) and triethylsilane (364.00 mg, 3.13 mmol, 0.5 mL, 1.78 eq) was added tert-butyl N-[(1R)-1-(2-imidazol-1-ylethylcarbamoyl)-3-oxo-3-(tritylamino)propyl]carbamate (1 g, 1.76 mmol, 1 eq). The mixture was stirred at 25° C. for 3 hr under N2 atmosphere. The reaction mixture was concentrated under reduced pressure to give the product (2R)-2-amino-N-(2-imidazol-1-ylethyl)butanediamide (590 mg, crude, TFA) as white solid.

### Step 3

To a solution of (2R)-2-amino-N-(2-imidazol-1-ylethyl)butanediamide (590 mg, 1.74 mmol, 1 eq, TFA) in DMF (5 mL) was added DIEA (674.27 mg, 5.22 mmol, 908.72 uL, 3 eq) and octanoyl octanoate (564.30 mg, 2.09 mmol, 1.2 eq). The mixture was stirred at 25° C. for 1 hr. The reaction mixture was concentrated under reduced pressure to give a residue. The residue was purified by prep-HPLC ([water(10 mM NH₄HCO₃)-ACN]) to give compound (2R)-N-(2-imidazol-1-ylethyl)-2-(octanoylamino)butanediamide (104 mg, 290.00 umol, 16.68% yield, 98% purity) as white solid.

### Example 201: (R)-2-octanamido-N′-(2-(pyridin-3-yl)ethyl)succinamide

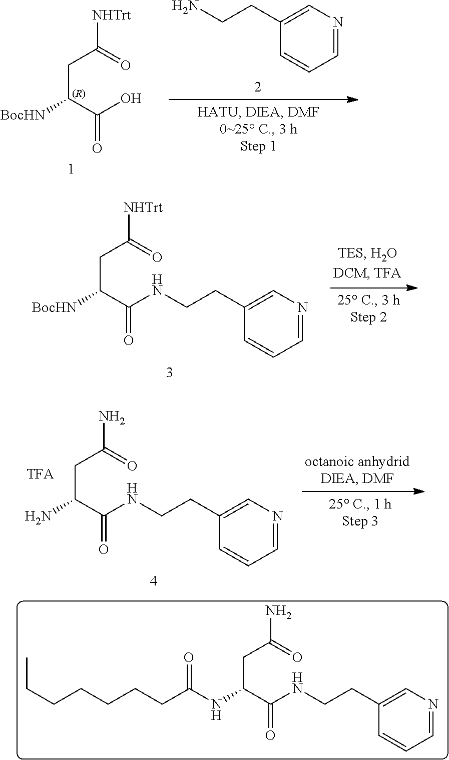

### Step 1

To a solution of (2R)-2-(tert-butoxycarbonylamino)-4-oxo-4-(tritylamino)butanoic acid (1 g, 2.11 mmol, 1 eq) and 2-(3-pyridyl)ethanamine (308.93 mg, 2.53 mmol, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 3 hr. LC-MS showed the desired compound was detected. The reaction mixture was poured to water (20 ml), filtered and the filter cake was concentrated under reduced pressure to give the product tert-butyl N-[(1R)-3-oxo-1-[2-(3-pyridyl) ethylcarbamoyl]-3-(tritylamino)propyl]carbamate (1 g, 1.66 mmol, 78.72% yield, 96% purity) as white solid.

### Step 2

A mixture of DCM (1.32 g, 15.54 mmol, 1 mL, 9.99 eq), TFA (27.72 g, 243.11 mmol, 18 mL, 156.32 eq), H₂O (500.00 mg, 27.75 mmol, 0.5 mL, 17.85 eq) and triethylsilane (364.00 mg, 3.13 mmol, 0.5 mL, 2.01 eq) was added tert-butyl N-[(1R)-3-oxo-1-[2-(3-pyridyl)ethylcarbamoyl]-3-(tritylamino)propyl]carbamate (900 mg, 1.56 mmol, 1 eq). The mixture was stirred at 25° C. for 3 hr under N2 atmosphere. The reaction mixture was concentrated under reduced pressure to give the crude product (2R)-2-amino-N-[2-(3-pyridyl)ethyl]butanediamide (540 mg, crude, TFA) as white solid.

### Step 3

To a solution of (2R)-2-amino-N-[2-(3-pyridyl)ethyl]butanediamide (540 mg, 1.54 mmol, 1 eq, TFA) in DMF (5 mL) was added DIEA (597.71 mg, 4.62 mmol, 805.54 uL, 3 eq) and octanoyl octanoate (500.22 mg, 1.85 mmol, 1.2 eq). The mixture was stirred at 25° C. for 1 hr. The reaction mixture was concentrated under reduced pressure to give a residue. The residue was purified by prep-HPLC ([water(10 mM NH₄HCO₃)-ACN]) to give compound (2R)-2-(octanoylamino)-N-[2-(3-pyridyl)ethyl]butanediamide (15 mg, 39.31 umol, 2.55% yield, 95% purity) as white solid.

### Example 202: (R)-N′-(2-(furan-2-yl)ethyl)-2-octanamidosuccinamide

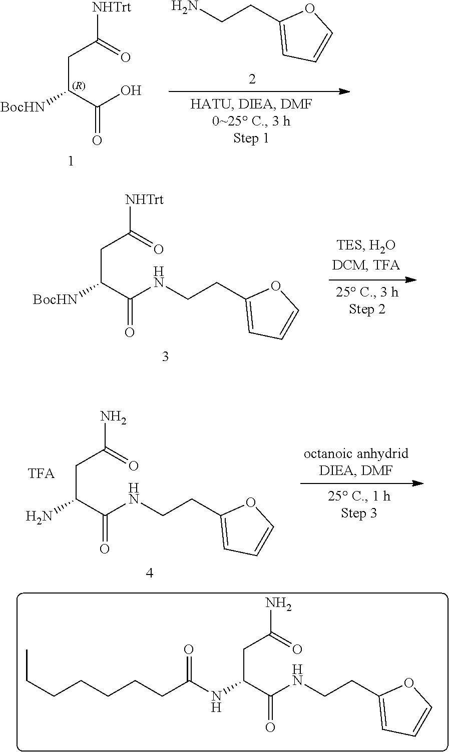

### Step 1

A mixture of (2R)-2-(tert-butoxycarbonylamino)-4-oxo-4-(tritylamino)butanoic acid (1 g, 2.11 mmol, 1 eq), 2-(2-furyl)ethanamine (281.05 mg, 2.53 mmol, 1.2 eq) and HATU (961.50 mg, 2.53 mmol, 1.2 eq) in DMF (10 mL) was added DIEA (599.17 mg, 4.64 mmol, 807.51 uL, 2.2 eq) at 0° C. Then the mixture was stirred at 25° C. for 3 hr under N₂ atmosphere. The reaction mixture was quenched by addition H₂O 100 mL at 0° C., and then diluted with H₂O 200 mL and extracted with EtOAc 900 mL (300 mL*3). The combined organic layers were washed with brine 50 mL dried over Na₂SO₄, filtered and concentrated under reduced pressure to give a residue to give compound tert-butyl N-[(1R)-1-[2-(2-furyl)ethylcarbamoyl]-3-oxo-3-(tritylamino)propyl]carbamate (500 mg, 854.36 umol, 40.54% yield, 97% purity) as a white solid.

### Step 2

A mixture of tert-butyl N-[(1R)-1-[2-(2-furyl)ethylcarbamoyl]-3-oxo-3-(tritylamino) propyl]carbamate (0.5 g, 854.36 umol, 40.54% yield, 97% purity, 1 eq), TFA (18 mL), TESH (1 mL), DCM (1 mL) and H₂O (0.5 mL) was stirred at 25° C. for 5 hr under N2 atmosphere. The reaction mixture was concentrated under reduced pressure to give compound (2R)-2-amino-N-[2-(2-furyl)ethyl]butanediamide (150 mg, crude, TFA) as a white solid.

### Step 3

A mixture of (2R)-2-amino-N-[2-(2-furyl)ethyl]butanediamide (150 mg, 442.13 umol, 1 eq, TFA) octanoyl octanoate (179.33 mg, 663.19 umol, 1.5 eq) and DIEA (57.14 mg, 442.13 umol, 77.01 uL, 1 eq) in DMF (5 mL) was stirred at 25° C. for 5 hr under N₂ atmosphere. The residue was purified by prep-HPLC (column: Welch Xtimate C18 100*25 mm*3 um; mobile phase: [water(0.05% HCl)-ACN]; B %: 30%-60%, 8 min) to give compound (2R)-N-[2-(2-furyl)ethyl]-2-(octanoylamino)butanediamide (6 mg, 16.73 umol, 3.78% yield, 98% purity) as a white solid.

### Example 203: (R)-2-octanamido-N′-(2-(thiazol-5-yl)ethyl)succinamide

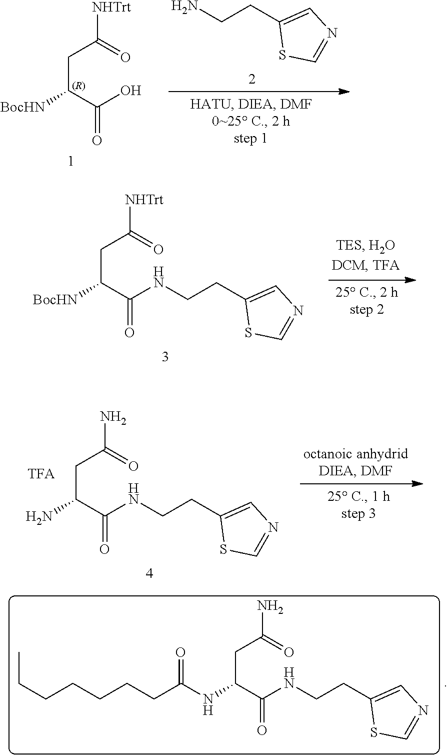

### Step 1

To a solution of (2R)-2-(tert-butoxycarbonylamino)-4-oxo-4-(tritylamino)butanoic acid (300 mg, 632.18 umol, 1 eq) and 2-thiazol-5-ylethanamine (152.57 mg, 758.62 umol, 1.2 eq, 2HCl) in DMF (5 mL) was added HATU (288.45 mg, 758.62 umol, 1.2 eq) and DIPEA (245.11 mg, 1.90 mmol, 330.34 uL, 3 eq) at 0° C. The mixture was stirred at 25° C. for 2 h. LCMS showed the starting reactant was consumed. The reaction mixture was quenched by addition H₂O 20 mL at 0° C., the precipitate was filtered and the filter cake was concentrated under reduced pressure to give compound tert-butyl N-[(1R)-3-oxo-1-(2-thiazol-5-ylethylcarbamoyl)-3-(tritylamino) propyl]carbamate (350 mg, 598.57 umol, 94.68% yield) as a white solid.

### Step 2

The mixture of tert-butyl N-[(1R)-3-oxo-1-(2-thiazol-5-ylethylcarbamoyl)-3-(tritylamino) propyl]carbamate (350 mg, 598.57 umol, 1 eq), TES (0.25 mL), H₂O (0.25 mL), DCM (0.5 mL), TFA (9 mL) was stirred at 25° C. for 2 h. The mixture was concentrated under reduced pressure to give compound (2R)-2-amino-N-(2-thiazol-5-ylethyl)butanediamide (210 mg, crude, TFA) was obtained as a white solid.

### Step 3

To a solution of (2R)-2-amino-N-(2-thiazol-5-ylethyl)butanediamide (210 mg, 589.36 umol, 1 eq, TFA) and octanoyl octanoate (239.05 mg, 884.04 umol, 1.5 eq) in DMF (5 mL) was added DIPEA (228.51 mg, 1.77 mmol, 307.97 uL, 3 eq). The mixture was stirred at 25° C. for 1 h. The mixture was concentrated under reduced pressure to give a residue. The residue was purified by prep-HPLC (column: Phenomenex Luna C18 100*30 mm*5 um; mobile phase: [water(0.04% HCl)-ACN]; B %: 20%-50%, 10 min) to give compound (2R)-2-(octanoylamino)-N-(2-thiazol-5-ylethyl)butanediamide (103 mg, 254.35 umol, 43.16% yield, 100% purity, HCl) as a white solid.

### Example 204: (R)-N′-(2-(1H-tetrazol-5-yl)ethyl)-2-octanamidosuccinamide

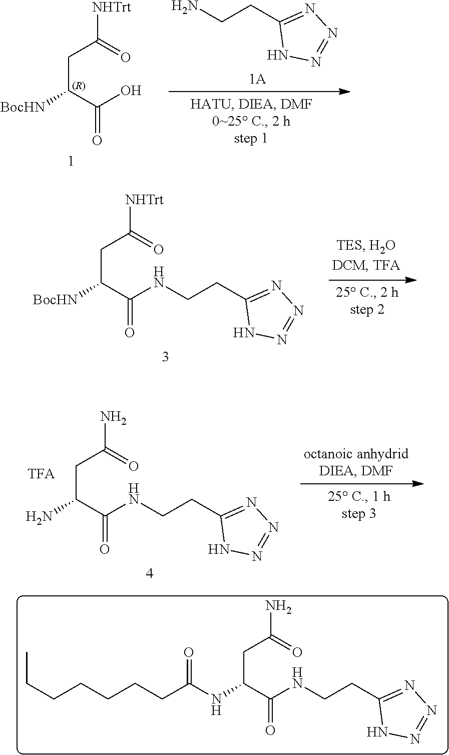

### Step 1

To a solution of (2R)-2-(tert-butoxycarbonylamino)-4-oxo-4-(tritylamino)butanoic acid (500 mg, 1.05 mmol, 1 eq) and 2-(1H-tetrazol-5-yl)ethanamine (143.03 mg, 1.26 mmol, 1.2 eq) in DMF (5 mL) was added HATU (480.75 mg, 1.26 mmol, 1.2 eq) and DIPEA (299.59 mg, 2.32 mmol, 403.75 uL, 2.2 eq) at 0° C. The mixture was stirred at 25° C. for 2 h. LCMS showed the starting reactant was consumed. The reaction mixture was quenched by addition H2O 20 mL at 0° C., the precipitate was filtered and the filter cake was concentrated under reduced pressure to give compound tert-butyl N-[(1R)-3-oxo-1-[2-(1H-tetrazol-5-yl)ethylcarbamoyl]-3-(tritylamino)propyl]carbamate (550 mg, 965.50 umol, 91.64% yield) as a white solid.

### Step 2

The tert-butyl N-[(1R)-3-oxo-1-[2-(1H-tetrazol-5-yl)ethylcarbamoyl]-3-(tritylamino)propyl]carbamate (550 mg, 965.50 umol, 1 eq) was dissolved with TES (0.5 mL), H₂O (0.5 mL), DCM (1 mL) and TFA (18 mL). The mixture was stirred at 25° C. for 2 h. LCMS showed the starting reactant consumed. The mixture was concentrated under reduced pressure to give compound (2R)-2-amino-N-[2-(1H-tetrazol-5-yl)ethyl]butanediamide (330 mg, crude, TFA) as yellow oil.

### Step 3

To a solution of octanoyl octanoate (392.24 mg, 1.45 mmol, 1.5 eq) and (2R)-2-amino-N-[2-(1H-tetrazol-5-yl)ethyl]butanediamide (330 mg, 967.04 umol, 1 eq, TFA) in DMF (2 mL) was added DIEA (374.94 mg, 2.90 mmol, 505.31 uL, 3 eq). The reaction mixture was stirred at 25° C. for 1 h. LCMS showed the starting reactant consumed. The mixture was concentrated under reduced. The residue was purified by prep-HPLC (column: Phenomenex Luna C18 100*30 mm*5 um; mobile phase: [water(10 mM NH₄HCO₃)-ACN]; B %: 15%-45%, 10 min) to give compound (2R)-2-(octanoylamino)-N-[2-(1H-tetrazol-5-yl)ethyl]butanediamide (62 mg, 159.02 umol, 16.44% yield, 100% purity, HCl) as a white solid.

### Example 205: (R)-N′-(2-(4H-1,2,4-triazol-4-yl)ethyl)-2-octanamidosuccinamide

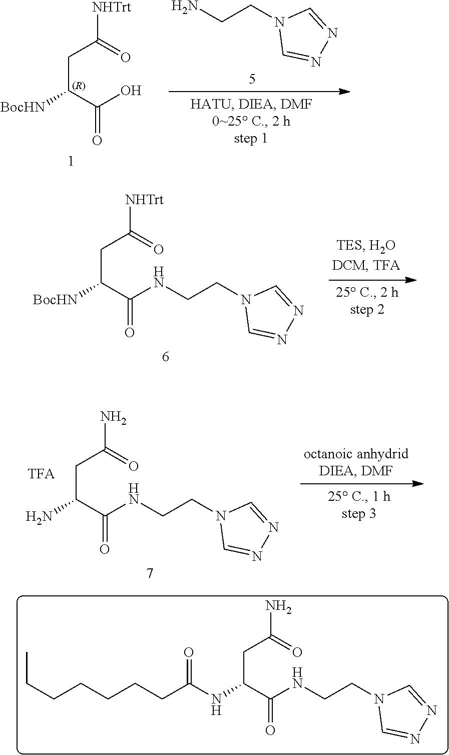

### Step 1

To a solution of (2R)-2-(tert-butoxycarbonylamino)-4-oxo-4-(tritylamino)butanoic acid (200 mg, 421.45 umol, 1 eq) and 2-(1,2,4-triazol-4-yl)ethanamine (56.71 mg, 505.74 umol, 1.2 eq) in DMF (5 mL) was added HATU (192.30 mg, 505.74 umol, 1.2 eq) and DIPEA (119.83 mg, 927.20 umol, 161.50 uL, 2.2 eq) at 0° C. The mixture was stirred at 25° C. for 2 h. The reaction mixture was quenched by addition H₂O 30 mL at 0° C. The precipitate was filtered and the filter cake was concentrated under reduced pressure to give compound tert-butyl N-[(1R)-3-oxo-1-[2-(1,2,4-triazol-4-yl)ethylcarbamoyl]-3-(tritylamino)propyl]carbamate (200 mg, 351.70 umol, 83.45% yield) as a white solid.

### Step 2

The tert-butyl N-[(1R)-3-oxo-1-[2-(1,2,4-triazol-4-yl)ethylcarbamoyl]-3-(tritylamino)propyl]carbamate (200 mg, 351.70 umol, 1 eq), TES (0.1 mL), H₂O (0.1 mL), DCM (0.2 mL) and TFA (3.6 mL) was stirred at 25° C. for 2 h. The mixture was concentrated under reduced pressure to give compound (2R)-2-amino-N-[2-(1,2,4-triazol-4-yl)ethyl]butanediamide (180 mg, crude, TFA) as a white solid.

### Step 3

To a solution of (2R)-2-amino-N-[2-(1,2,4-triazol-4-yl)ethyl]butanediamide (180 mg, 529.01 umol, 1 eq, TFA) and octanoyl octanoate (214.57 mg, 793.52 umol, 1.5 eq) in DMF (3 mL) was added DIPEA (205.11 mg, 1.59 mmol, 276.43 uL, 3 eq). The mixture was stirred at 25° C. for 1 h. The mixture was concentrated under reduced pressure to give a residue. The residue was purified by prep-HPLC (column: Phenomenex Luna C18 100*30 mm*5 um; mobile phase: [water(0.04% HCl)-ACN]; B %: 40%-70%, 10 min), the product in mobile phase was adjusted PH=7 by NaHCO₃, then precipitation product was further triturated with EtOAc at 25° C. for 30 min, then filtered and the filter cake was concentrated under reduced pressure to give compound (2R)-2-(octanoylamino)-N-[2-(1,2,4-triazol-4-yl)ethyl]butanediamide (21 mg, 58.99 umol, 11.15% yield, 99% purity) as a white solid.

### Example 206: (R)-2-octanamido-N′-(2-(6-oxo-1,6-dihydropyridin-3-yl)ethyl)succinamide

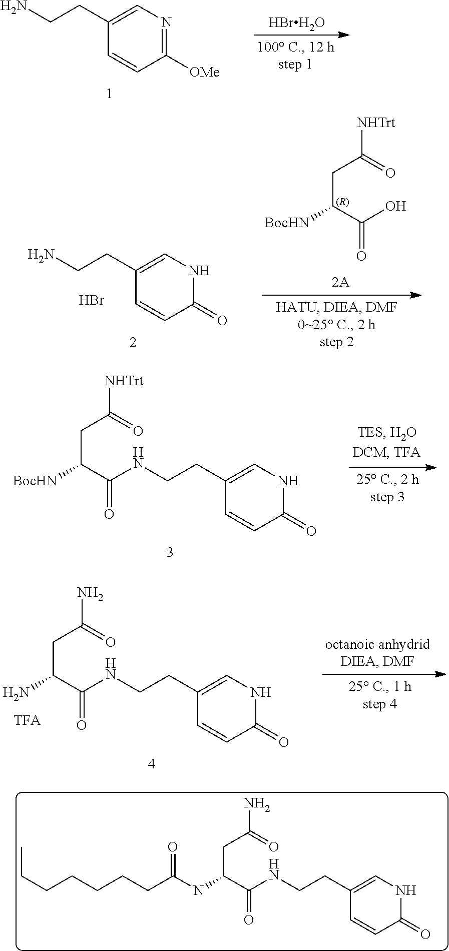

### Step 1

The 2-(6-methoxy-3-pyridyl)ethanamine (200 mg, 1.31 mmol, 1 eq) in HBr (5.96 g, 35.36 mmol, 4.00 mL, 48% purity, 26.91 eq) was stirred at 100° C. for 12 hr. The mixture was concentrated under reduced pressure to give compound 5-(2-aminoethyl)-1H-pyridin-2-one (280 mg, crude, HBr) as a brown solid.

### Step 2

To a solution of (2R)-2-(tert-butoxycarbonylamino)-4-oxo-4-(tritylamino)butanoic acid (300 mg, 632.18 umol, 1 eq) and 5-(2-aminoethyl)-1H-pyridin-2-one (166.20 mg, 758.62 umol, 1.2 eq, HBr) in DMF (8 mL) was added HATU (288.45 mg, 758.62 umol, 1.2 eq) and DIPEA (179.75 mg, 1.39 mmol, 242.25 uL, 2.2 eq) at 0° C. The mixture was stirred at 25° C. for 2 hr. The mixture was concentrated under reduced pressure to give a residue. The residue was purified by column chromatography (SiO₂, EtOAc/MeOH=1/0 to 2/1) to give compound tert-butyl N-[(1R)-3-oxo-1-[2-(6-oxo-1H-pyridin-3-yl)ethylcarbamoyl]-3-(tritylamino) propyl]carbamate (330 mg, 554.90 umol, 87.78% yield) as a white solid.

### Step 3

The tert-butyl N-[(1R)-3-oxo-1-[2-(6-oxo-1H-pyridin-3-yl)ethylcarbamoyl]-3-(tritylamino)propyl]carbamate (330 mg, 554.90 umol, 1 eq) in TES (0.05 mL), H₂O (0.05 mL), DCM (0.1 mL) and TFA (3.6 mL) was stirred at 25° C. for 2 hr. The mixture was concentrated under reduced pressure to give compound (2R)-2-amino-N-[2-(6-oxo-1H-pyridin-3-yl)ethyl]butanediamide (200 mg, crude, TFA) as a white solid.

### Step 4

To a solution of (2R)-2-amino-N-[2-(6-oxo-1H-pyridin-3-yl)ethyl]butanediamide (200 mg, 546.01 umol, 1 eq, TFA) and octanoyl octanoate (221.47 mg, 819.02 umol, 1.5 eq) in DMF (3 mL) was added DIPEA (211.70 mg, 1.64 mmol, 285.32 uL, 3 eq). The mixture was stirred at 25° C. for 1 hr. LCMS showed the starting reactant was consumed and have the desired. The mixture was concentrated under reduced pressure to give a residue. The crude product was purified by re-crystallization with EtOAc (5 mL) at 25° C. The precipitate was filtered and concentrated under reduced pressure to give compound (2R)-2-(octanoylamino)-N-[2-(6-oxo-1H-pyridin-3-yl)ethyl]butanediamide (135 mg, 356.70 umol, 65.33% yield) as a white solid.

### Example 207: (R)-2-(2,2-difluorooctanamido)-N′-(4-hydroxyphenethyl)succinamide

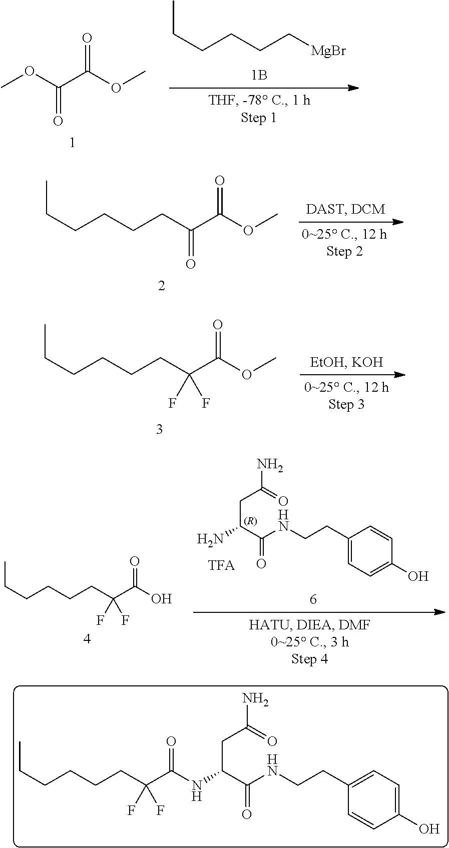

### Step 1

To a solution of dimethyl oxalate (2 g, 16.94 mmol, 1 eq) in THF (20 mL) was added bromo(hexyl)magnesium (1 M, 20.32 mL, 1.2 eq) at −78° C. The mixture was stirred at −78° C. for 1 hr. TLC indicated the reaction was completed. The reaction mixture was neutralized with 1N HCl to pH=7, and then extracted with ethyl acetate 120 mL (40 mL*3). The combined organic layers were dried over Na₂SO₄, filtered and the filtrate was concentrated under reduced pressure to give compound methyl 2-oxooctanoate (600 mg, 3.48 mmol, 20.57% yield) as colorless oil.

### Step 2

To a solution of methyl 2-oxooctanoate (500 mg, 2.90 mmol, 1 eq) in DCM (10 mL) was added DAST (2.34 g, 14.52 mmol, 1.92 mL, 5 eq) at 0° C. The mixture was stirred at 25° C. for 12 hr. TLC indicated methyl 2-oxooctanoate was consumed completely. The reaction mixture was quenched by H₂O (10 mL), then added NaHCO₃ aq. to adjust PH=7, extracted with DCM 90 mL (30 mL*3). The combined organic layers were dried over Na₂SO₄, filtered and the filtrate was concentrated under reduced pressure to give the product methyl 2,2-difluorooctanoate (300 mg, 1.54 mmol, 53.20% yield) as yellow oil.

### Step 3

To a solution of methyl 2,2-difluorooctanoate (300 mg, 1.54 mmol, 1 eq) in EtOH (10 mL) was added KOH (866.64 mg, 15.45 mmol, 10 eq) at 0° C. The mixture was stirred at 25° C. for 12 hr. TLC indicated methyl 2,2-difluorooctanoate was consumed completely. The reaction mixture was diluted by H₂O (30 mL), and then added 1N HCl to adjust PH=2, extracted with ethyl acetate 120 mL (40 mL*3). The combined organic layers were dried over Na₂SO₄, filtered and the filtrate concentrated under reduced pressure to give compound 2,2-difluorooctanoic acid (200 mg, 1.11 mmol, 71.86% yield) as yellow oil.

### Step 4

To a solution of 2,2-difluorooctanoic acid (200 mg, 1.11 mmol, 1 eq) and (2R)-2-amino-N-[2-(4-hydroxyphenyl)ethyl]butanediamide (486.55 mg, 1.33 mmol, 1.2 eq, TFA) in DMF (10 mL) was added HATU (506.43 mg, 1.33 mmol, 1.2 eq) and DIEA (315.58 mg, 2.44 mmol, 425.31 uL, 2.2 eq) at 0° C. The mixture was stirred at 25° C. for 3 hr. The reaction mixture was concentrated under reduced pressure to give a residue. The residue was purified by prep-HPLC ([water(10 mM NH₄HCO₃)-ACN]) to give compound (2R)-2-(2,2-difluorooctanoylamino)-N-[2-(4-hydroxyl phenyl)ethyl]butanediamide (122 mg, 295.07 umol, 24.40% yield) as white solid.

### Example 208: (R)-N′-(2-(6-methoxypyridin-3-yl)ethyl)-2-octanamidosuccinamide

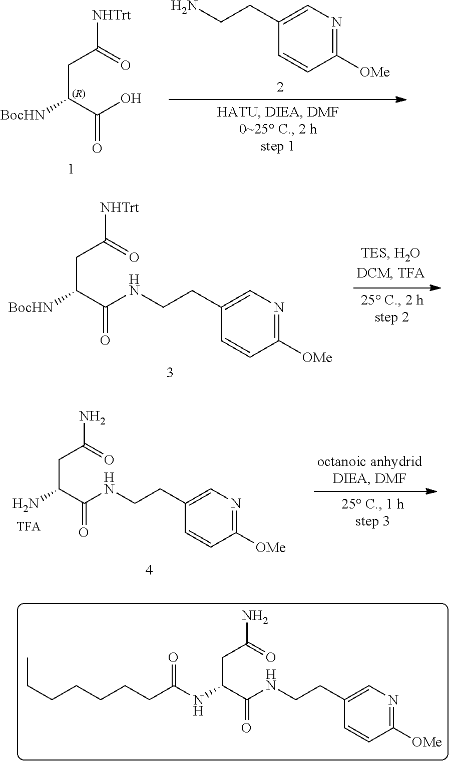

### Step 1

To a solution of (2R)-2-(tert-butoxycarbonylamino)-4-oxo-4-(tritylamino)butanoic acid (1 g, 2.11 mmol, 1 eq) and 2-(6-methoxy-3-pyridyl)ethanamine (384.86 mg, 2.53 mmol, 1.2 eq) in DMF (5 mL) was added HATU (961.50 mg, 2.53 mmol, 1.2 eq) and DIPEA (599.17 mg, 4.64 mmol, 807.51 uL, 2.2 eq) at 0° C. The mixture was stirred at 25° C. for 2 hr. LCMS showed the starting reactant was consumed. The reaction mixture was quenched by addition H₂O 30 mL at 0° C., the precipitate was filtered and the filter cake was concentrated under reduced pressure to give a compound tert-butyl N-[(1R)-1-[2-(6-methoxy-3-pyridyl) ethylcarbamoyl]-3-oxo-3-(tritylamino)propyl]carbamate (1.1 g, 1.81 mmol, 85.75% yield) as white solid.

### Step 2

A solution of tert-butyl N-[(1R)-1-[2-(6-methoxy-3-pyridyl)ethylcarbamoyl]-3-oxo-3-(tritylamino) propyl]carbamate (1.1 g, 1.81 mmol, 1 eq), TES (0.5 mL), H₂O (0.5 mL), DCM (1 mL) and TFA (18 mL) was stirred at 25° C. for 2 hr. LCMS showed the starting reactant was consumed. The mixture was concentrated under reduced pressure to give compound (2R)-2-amino-N-[2-(6-methoxy-3-pyridyl)ethyl]butanediamide (680 mg, crude, TFA) as a white solid.

### Step 3

To a solution of (2R)-2-amino-N-[2-(6-methoxy-3-pyridyl)ethyl]butanediamide (370 mg, 972.87 umol, 1 eq, TFA) and octanoyl octanoate (394.60 mg, 1.46 mmol, 1.5 eq) in DMF (5 mL) was added DIPEA (377.21 mg, 2.92 mmol, 508.37 uL, 3 eq). The mixture was stirred at 25° C. for 1 hr. LCMS showed the starting reactant was consumed. The mixture was concentrated under reduced pressure to give a residue. The residue was purified by prep-HPLC (column: Welch Xtimate C18 100*25 mm*3 um; mobile phase: [water(0.05% HCl)-ACN]; B %: 5%-35%, 8 min) to give compound (2R)-N-[2-(6-methoxy-3-pyridyl)ethyl]-2-(octanoylamino) butanediamide (120 mg, 279.75 umol, 28.76% yield, HCl) as a white solid.

### Example 209: (R,E)-2-(4-cycloheptylbut-2-enamido)-N′-phenethylsuccinamide and (R)-2-(4-cycloheptylbutanamido)-N1-phenethylsuccinamide

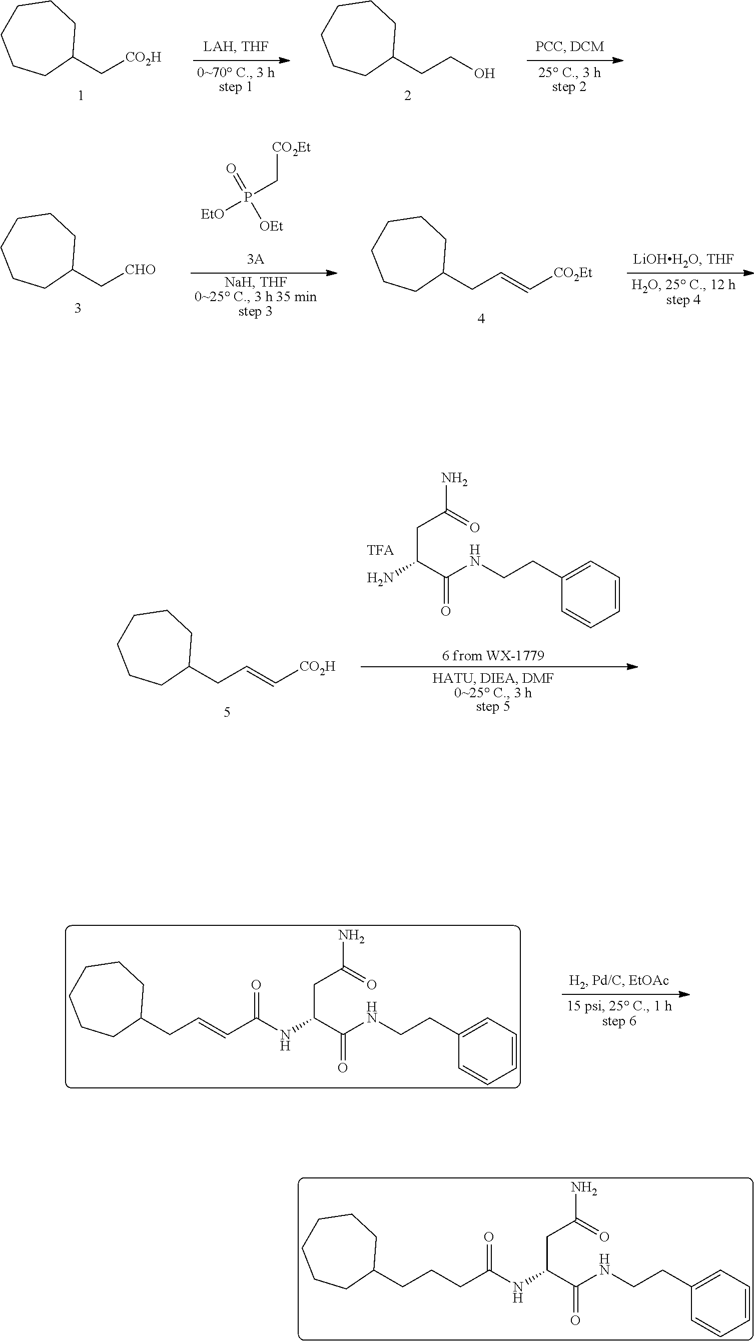

### Step 1

2-cycloheptylacetic acid (0.5, 3.20 mmol, 1 eq) was dissolved in THF (25 mL) under N₂, and the resulting solution was added LAH (364.43 mg, 9.60 mmol, 3 eq) slowly in portions at 0° C. Then the reaction mixture was allowed to warm to 25° C. and refluxing for 3 hr at 70° C. The reaction mixture was added water (5 mL). Then diluted with EtOAc (75 mL) and subsequently acidified by addition of 4 M HCl (25 mL) to dissolve the precipitate. The organic phase was separated and washed with sat. NaHCO₃ aq. (2* 75 mL), dried over anhydrous Na₂SO₄, filtered and concentrated in vacuum to give compound 2-cycloheptylethanol (400 mg, crude) as colorless oil.

### Step 2

To a solution of 2-cycloheptylethanol (0.4 g, 2.81 mmol, 1 eq) in DCM (120 mL) was added PCC (909.27 mg, 4.22 mmol, 1.5 eq) under N2 at 25° C. The mixture was stirred at 25° C. for 3 hr. Desired mass was detected by LCMS. The reaction mixture was diluted with EtOAc(120 mL) and stirred at 25° C. for 1 hr, and then filtered through a pad of celite and silica (1:1). The filtrate was dried with anhydrous Na₂SO₄, filtered and concentrated in vacuum to get compound 2-cycloheptylacetaldehyde (0.5 g, crude) as yellow oil.

### Step 3

To a solution of NaH (185.40 mg, 4.64 mmol, 60% purity, 1.3 eq) in THF (10 mL) was added ethyl 2-diethoxyphosphorylacetate (879.35 mg, 3.92 mmol, 778.19 uL, 1.1 eq) drop wise at 0° C. After 5 min, 2-cycloheptylacetaldehyde (500 mg, 3.57 mmol, 1 eq) in THF (5 mL) was added and the mixture was stirred at 0° C. for 30 min. The reaction mixture was allowed to warm to 25° C. and stirred for 3 hr. LCMS showed the reaction was completed and desired mass was detected. The reaction mixture was quenched by water (30 mL), and then extracted with EtOAc (40 mL*3), washed with brine (60 mL), dried with anhydrous Na₂SO₄, filtered and concentrated in vacuum to get compound ethyl 4-cycloheptylbut-2-enoate (560 mg, crude) as a yellow oil.

### Step 4

To a mixture of ethyl 4-cycloheptylbut-2-enoate (560 mg, 2.66 mmol, 1 eq) in THF (2 mL), EtOH (2 mL) and H₂O (2 mL) was added LiOH·H₂O (558.64 mg, 13.31 mmol, 5 eq) in one portion at 25° C. The mixture was stirred at 25° C. for 12 hours. LCMS showed desired mass was detected. The reaction mixture was treated with water (30 mL). The organic phase was separated. The aqueous phase was adjusted to pH=3 with 1 N HCl, then extracted with EtOAc(20 mL*3), the combined organic phase was dried with anhydrous Na₂SO₄, filtered and concentrated in vacuum to give compound 4-cycloheptylbut-2-enoic acid (320 mg, 1.70 mmol, 62.08% yield, 97% purity) as a yellow oil.

### Step 5

To a solution of 4-cycloheptylbut-2-enoic acid (110 mg, 603.54 umol, 1 eq) and (2R)-2-amino-N-(2-phenylethyl)butanediamide (316.23 mg, 724.25 umol, 80% purity, 1.2 eq, TFA) in DMF (1.5 mL) was added HATU (275.38 mg, 724.25 umol, 1.2 eq) and DIPEA (171.60 mg, 1.33 mmol, 231.27 uL, 2.2 eq) at 0° C. The mixture was stirred at 25° C. for 3 hrs. The reaction mixture was treated with water (15 mL) and the precipitate was filtered and concentrated under reduced pressure to get the crude product as a white solid. The crude product was purified by p-HPLC (column: Waters Xbridge BEH C18 100*30 mm*10 um; mobile phase: [water(10 mM NH₄HCO₃)-ACN]; B %: 30%-60%, 8 min) to give compound (2R)-2-(4-cycloheptylbut-2-enoylamino)-N-(2-phenylethyl)butanediamide (13 mg, 98% purity) as a white solid.

### Step 6

To a solution of (2R)-2-(4-cycloheptylbut-2-enoylamino)-N-[2-(4-hydroxyphenyl)ethyl]utanediamide (50 mg, 120.33 umol, 1 eq) in EtOAc (3.75 mL) was added Pd/C (0.1 g, 10% purity) under Ar. Then the mixture was degassed under reduced pressure and purged with H₂ three times, and the mixture was stirred at 25° C. under H₂ for 1 hr at 15 psi. Desired mass was detected by LCMS. The reaction mixture was filtered through a pad of celite in vacuum to get a residue. The residue was purified by prep-HPLC (column: Waters Xbridge BEH C18 100*25 mm*5 um; mobile phase: [water (10 mM NH4HCO3)-ACN]; B %: 30%-60%, 10 min) to get compound (2R)-2-(4-cycloheptylbutanoylamino)-N-(2-phenylethyl) butanediamide (72 mg, 95.88% purity) as a white solid.

### Example 210: (R,E)-2-(4-cycloheptylbut-2-enamido)-N1-(4-hydroxyphenethyl)succinamide and (R)-2-(4-cycloheptylbutanamido)-N1-(4-hydroxyphenethyl)succinamide

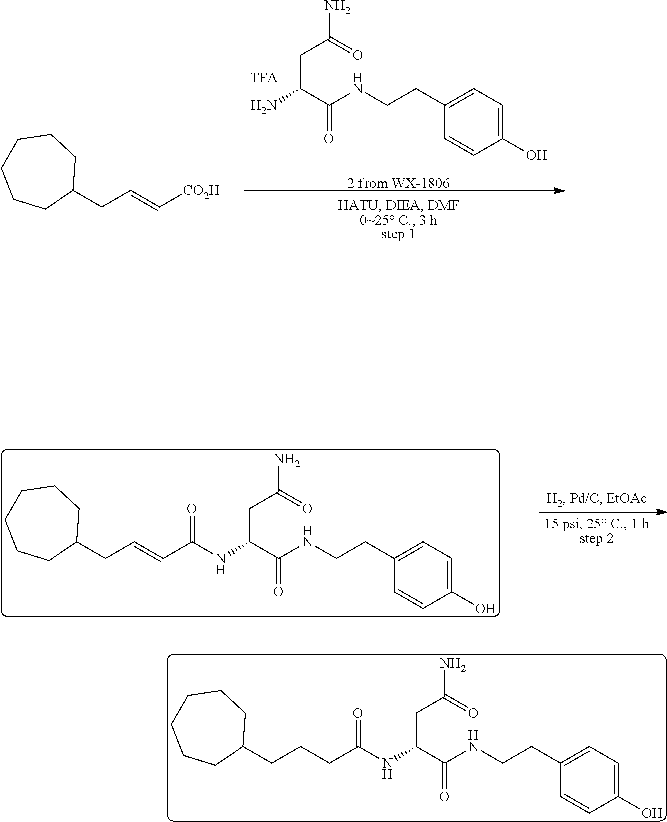

### Step 1

To a solution of 4-cycloheptylbut-2-enoic acid (110 mg, 603.54 umol, 1 eq) and (2R)-2-amino-N-[2-(4-hydroxyphenyl)ethyl]butanediamide (293.97 mg, 724.25 umol, 90% purity, 1.2 eq, TFA) in DMF (1.5 mL) was added HATU (275.38 mg, 724.25 umol, 1.2 eq) and DIPEA (171.60 mg, 1.33 mmol, 231.27 uL, 2.2 eq) at 0° C. The mixture was stirred at 25° C. for 3 hr. The residue was treated with water (10 mL) and the precipitate was filtered and concentrated under reduced pressure to give the crude product. The crude product was purified by prep-HPLC (column: Welch Xtimate C18 100*25 mm*3 um; mobile phase: [water(0.05% HCl)-ACN]; B %: 20%-45%, 8 min) to give compound (2R)-2-(4-cycloheptylbut-2-enoylamino)-N-[2-(4-hydroxyl phenyl)ethyl]butane diamide (23 mg, 98.11% purity) as a white solid.

### Step 2

To a solution of (2R)-2-(4-cycloheptylbut-2-enoylamino)-N-[2-(4-hydroxyphenyl)ethyl]butanediamide (50 mg, 120.33 umol, 1 eq) in EtOAc (3.75 mL) was added Pd/C (0.1 g, 10% purity) under Ar. The mixture was degassed under reduced pressure and purged with H₂ three times and stirred at 25° C. for 1 hr under H₂ (15 psi). LCMS showed desired compound was detected. The reaction mixture was filtered under reduced pressure to get compound (2R)-2-(4-cycloheptylbutanoylamino)-N-[2-(4-hydroxyphenyl) ethyl]butanediamide (35 mg, 96.15% purity) as a white solid.

### Example 211: (R)-2-(3-cyclohexylpropanamido)-N1-(4-hydroxyphenethyl)succinamide

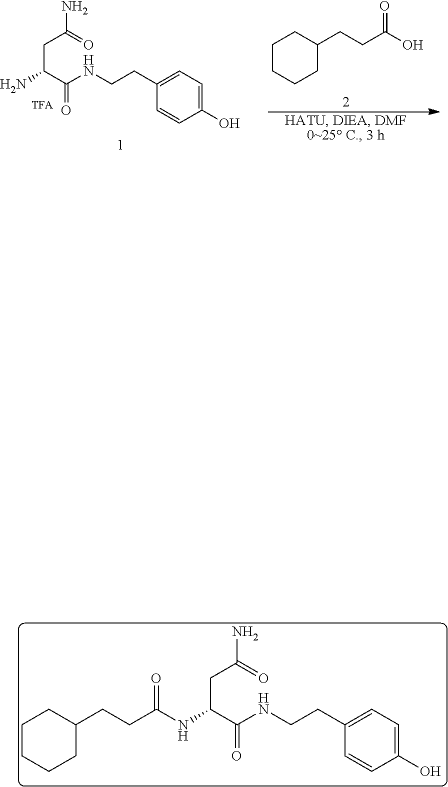

To a mixture of (2R)-2-amino-N-[2-(4-hydroxyphenyl)ethyl]butanediamide (300 mg, 517.38 umol, 63% purity, 1 eq, TFA) and 3-cyclohexylpropanoic acid (80.83 mg, 517.38 umol, 88.62 uL, 1 eq) in DMF (10 mL) was added DIPEA (468.06 mg, 3.62 mmol, 630.81 uL, 7 eq) and HATU (236.07 mg, 620.85 umol, 1.2 eq) at 0° C. under N₂. The mixture was stirred at 25° C. for 3 hour. LCMS showed the reaction was completed. The mixture was cooled to 0° C. and water (30 mL) was added slowly, then the precipitate was filtered and the filter cake was washed by water (50 mL*2). The filter cake was concentrated in vacuum. The residue was purified by p-HPLC (column: Kromasil C18 (250*50 mm*10 um); mobile phase: [water(10 mM NH4HCO3)-ACN]; B %: 25%-45%, 10 min) to give (2R)-2-(3-cyclohexylpropanoylamino)-N-[2-(4-hydroxyphenyl)ethyl]butanediamide (57 mg, 141.96 umol, 27.44% yield, 97% purity) as a white solid.

### Example 212: (R)-2-(3-cyclohexylpropanamido)-N′-phenethylsuccinamide

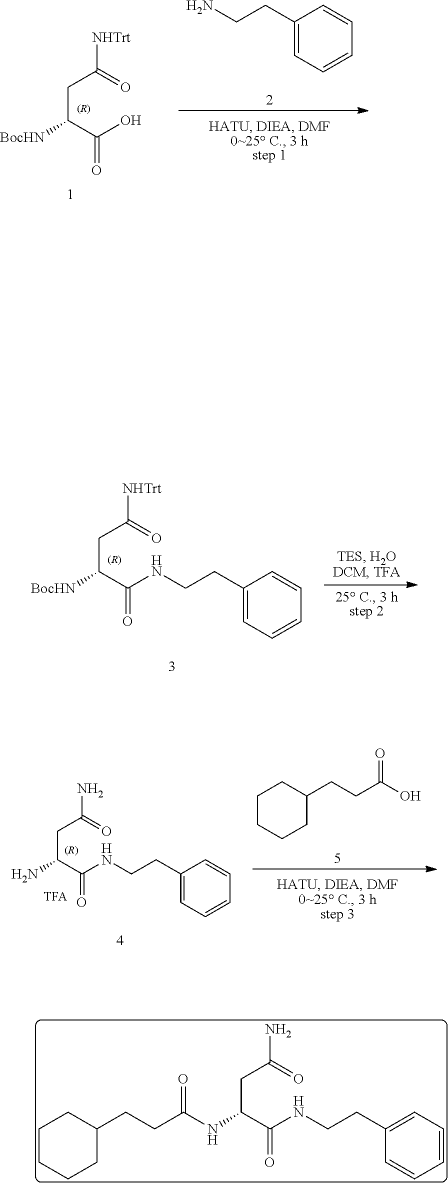

### Step 1

To a solution of (2R)-2-(tert-butoxycarbonylamino)-4-oxo-4-(tritylamino)butanoic acid (6 g, 12.64 mmol, 1 eq) and 2-phenylethanamine (1.84 g, 15.17 mmol, 1.91 mL, 1.2 eq) in DMF (70 mL) was added HATU (5.77 g, 15.17 mmol, 1.2 eq) and DIPEA (3.60 g, 27.82 mmol, 4.85 mL, 2.2 eq) at 0° C. The mixture was stirred at 25° C. for 3 hr. TLC showed the reaction was completed and new spots formed. The reaction mixture was treated with H₂O (100 mL) at 0° C., and the precipitate was filtered and concentrated in vacuum to get compound tert-butyl N-[(1R)-3-oxo-1-(2-phenylethylcarbamoyl)-3-(tritylamino)propyl]carbamate (6.5 g, crude) as a white solid.

### Step 2

The tert-butyl N-[(1R)-3-oxo-1-(2-phenylethylcarbamoyl)-3-(tritylamino)propyl]carbamate (6.5 g, 11.25 mmol, 1 eq), TES (2.58 g, 11.25 mmol, 3.5 mL, 1 eq), H₂O (4.14 g, 229.60 mmol, 4.14 mL, 20.41 eq), DCM (10.92 g, 128.57 mmol, 8.27 mL, 11.43 eq) and TFA (229.32 g, 2.01 mol, 148.91 mL, 178.75 eq) was stirred at 25° C. for 3 hr. TLC showed two new spots formed and reactant was consumed completely. The reaction mixture was concentrated in vacuum to give compound (2R)-2-amino-N-(2-phenylethyl)butanediamide (4.6 g, crude, TFA) as a yellow solid.

### Step 3

To a solution of (2R)-2-amino-N-(2-phenylethyl)butanediamide (0.3 g, 343.54 umol, 40% purity, 1.2 eq, TFA) and 3-cyclohexylpropanoic acid (44.72 mg, 286.28 umol, 49.04 uL, 1 eq) in DMF (3 mL) was added HATU (130.62 mg, 343.54 umol, 1.2 eq) and DIPEA (185.00 mg, 1.43 mmol, 249.33 uL, 5 eq) at 0° C., and then the mixture was stirred at 25° C. for 3 hr. TLC showed new spots formed and the reaction was completed. The reaction mixture was treated with water (50 ml), and the precipitate was filtered and concentrated in vacuum to give compound (2R)-2-(3-cyclohexylpropanoylamino)-N-(2-phenylethyl) butanediamide (110 mg, 97.23% purity) as a white solid.

### Example 213: (R)-N′-((S)-1-(4-hydroxyphenyl)propan-2-yl)-2-octanamidosuccinamide and (R)-N′-((R)-1-(4-hydroxyphenyl)propan-2-yl)-2-octanamidosuccinamide

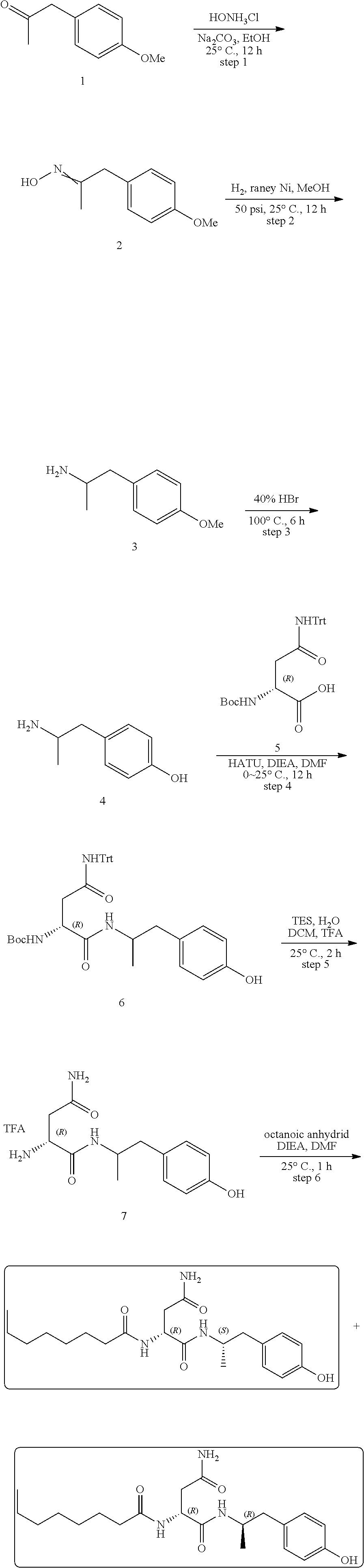

### Step 1

To a solution of 1-(4-methoxyphenyl)propan-2-one (2 g, 12.18 mmol, 1 eq) and NH₂OH·HCl (1.69 g, 24.36 mmol, 2 eq) in EtOH (30 mL) was added Na₂CO₃ (2.58 g, 24.36 mmol, 2 eq), stirred at 25° C. for 12 h. The mixture was added H₂O (50 mL), extracted with EtOAc (20 mL*3), the organic layer was washed with brine (20 mL*2), dried over Na₂SO₄, filtered and the filtrate was concentrated under reduced pressure to give the compound 1-(4-methoxyphenyl)propan-2-one oxime (2 g, crude) as yellow oil.

### Step 2

To a solution of Raney-Ni (2 g, 23.34 mmol, 2.09 eq) in MeOH (40 mL) was added 1-(4-methoxyphenyl)propan-2-one oxime (2 g, 11.16 mmol, 1 eq). The mixture was stirred at 25° C. for 12 h under H₂ under 50 psi. TLC showed the starting reactant consumed. The mixture was filtered and concentrated the filtrate under reduced pressure. The residue was purified by column chromatography (SiO₂, Petroleum ether/Ethyl acetate=3/1 to MeOH) to give the compound 1-(4-methoxyphenyl)propan-2-amine (1.2 g, crude) as a white solid.

### Step 3

A solution of 1-(4-methoxyphenyl)propan-2-amine (200 mg, 1.21 mmol, 1 eq) in HBr (3.23 g, 15.98 mmol, 2.17 mL, 40% purity, 13.20 eq) was stirred at 100° C. for 6 h. LCMS showed the starting reactant consumed. The mixture was concentrated under reduced pressure. The crude product was triturated with EtOAc at 25° C. for 15 min to give the compound 4-(2-aminopropyl) phenol (210 mg, 904.72 umol, 74.74% yield, HBr) as a gray white solid.

### Step 4

To a solution of (2R)-2-(tert-butoxycarbonylamino)-4-oxo-4-(tritylamino)butanoic acid (290 mg, 611.11 umol, 1 eq) and 4-(2-aminopropyl)phenol (210 mg, 904.72 umol, 1.48 eq, HBr) in DMF (5 mL) was added HATU (278.83 mg, 733.33 umol, 1.2 eq) and DIPEA (236.94 mg, 1.83 mmol, 319.33 uL, 3 eq) at 0° C. The mixture was stirred at 25° C. for 12 h. The mixture was poured into H₂O (10 mL), then filtered and filter cake was concentrated under reduced pressure to give the compound tert-butyl N-[1-[[2-(4-hydroxyphenyl)-1-methyl-ethyl]carbamoyl]-3-oxo-3-(tritylamino)propyl]carbamate (350 mg, crude) as a brown solid.

### Step 5

The tert-butyl N-[1-[[2-(4-hydroxyphenyl)-1-methyl-ethyl]carbamoyl]-3-oxo-3-(tritylamino) propyl]carbamate (350 mg, 575.91 umol, 1 eq) was added to a solution of TES (0.5 mL), H₂O (0.5 mL), DCM (1 mL) and TFA (18 mL) and stirred at 25° C. for 2 h. The mixture was concentrated under reduced pressure to give the compound (2R)-2-amino-N-[2-(4-hydroxyphenyl)-1-methyl-ethyl]butanediamide (200 mg, crude, TFA) as yellow solid.

### Step 6

To a mixture of octanoyl octanoate (213.86 mg, 790.87 umol, 1.5 eq) and (2R)-2-amino-N-[2-(4-hydroxyphenyl)-1-methyl-ethyl]butanediamide (200 mg, 527.24 umol, 1 eq, TFA) in DMF (3 mL) was added DIPEA (204.42 mg, 1.58 mmol, 275.50 uL, 3 eq). The mixture was stirred at 25° C. for 1 h. The mixture was concentrated under reduced pressure to give a residue. The residue was purified by prep-HPLC (column: Phenomenex luna C18 80*40 mm*3 um; mobile phase: [water(0.04% HCl)-ACN]; B %: 35%-40%, 7 min) to give

(2R)-N-[(1S)-2-(4-hydroxyphenyl)-1-methyl-ethyl]-2-(octanoylamino)butanediamide (27 mg, 66.90 umol, 12.69% yield, 97% purity) as a white solid.

(2R)-N-[(1R)-2-(4-hydroxyphenyl)-1-methyl-ethyl]-2-(octanoylamino)butanediamide (35 mg, 86.72 umol, 16.45% yield, 97% purity) as a white solid.

### Example 214: (R)-N′-(4-hydroxyphenethyl)-N4-methyl-2-octanamidosuccinamide and

(R)-N⁴-hydroxy-N′-(4-hydroxyphenethyl)-2-octanamidosuccinamide

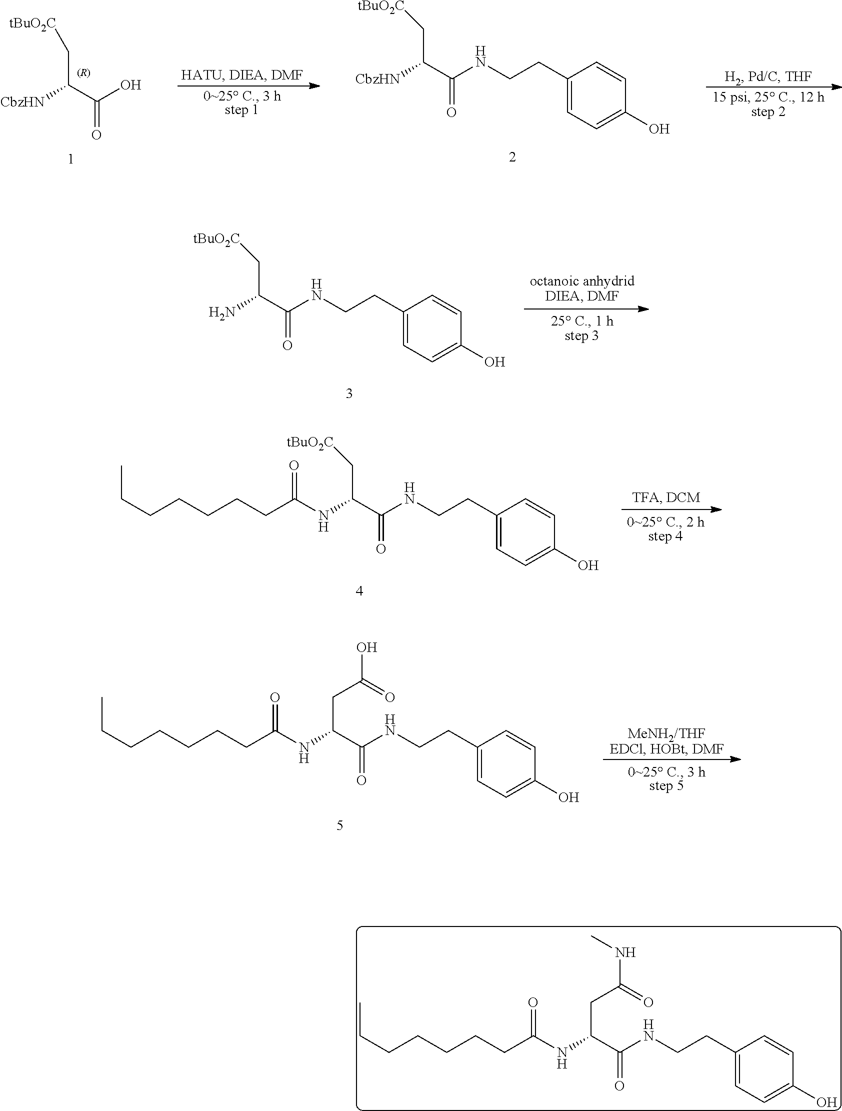

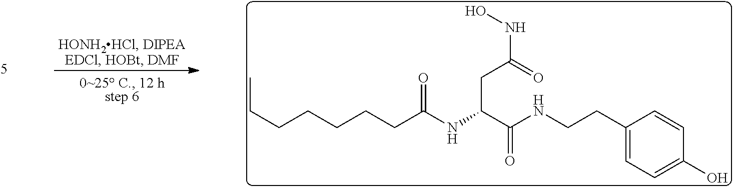

### Step 1

To a mixture of 4-(2-aminoethyl)phenol (1.21 g, 8.79 mmol, 1.2 eq) and (2R)-2-(benzyloxycarbonylamino)-4-tert-butoxy-4-oxo-butanoic acid hydrate (2.5 g, 7.32 mmol, 1 eq) in DMF (75 mL) was added HATU (3.34 g, 8.79 mmol, 1.2 eq) and DIPEA (2.08 g, 16.11 mmol, 2.81 mL, 2.2 eq) at 0° C. under N₂. The mixture was stirred at 25° C. for 3 hours. The mixture was added water (200 mL). The mixture was extracted with ethyl acetate (100 mL*2). The combined organic phase was washed with brine (200 mL), dried with anhydrous Na₂SO₄, filtered and concentrated in vacuum to get 3.05 g crude product as yellow oil.

### Step 2

To a solution of tert-butyl (3R)-3-(benzyloxycarbonylamino)-4-[2-(4-hydroxyphenyl) ethylamino]-4-oxo-butanoate (2.5 g, 5.65 mmol, 1 eq) in THF (100 mL) was added Pd/C (1 g, 5.65 mmol, 10% purity, 1.00 eq) under H₂. The mixture was stirred at 25° C. for 12 hr under H₂ (15 psi). TLC showed new spots formed. The reaction mixture was filtered through a pad of celite and concentrated in vacuum to give compound tert-butyl (3R)-3-amino-4-[2-(4-hydroxyphenyl) ethylamino]-4-oxo-butanoate (2 g, crude) as a yellow oil.

### Step 3

To a solution of tert-butyl (3R)-3-amino-4-[2-(4-hydroxyphenyl)ethylamino]-4-oxo-butanoate (1.5 g, 4.23 mmol, 87% purity, 1 eq) and octanoyl octanoate (1.26 g, 4.66 mmol, 1.1 eq) in DMF (25 mL) was added DIPEA (1.64 g, 12.70 mmol, 2.21 mL, 3 eq). The mixture was stirred at 25° C. for 1 hr. The reaction mixture was concentrated in vacuum to get a residue. The residue was purified by reversed-phase HPLC(column: 330 g Agela C18 column; mobile phase: A: Water; B: MEOH; B %: 45-75% 25 min; 75% 10 min, 70 ml/min) to give compound tert-butyl (3R)-4-[2-(4-hydroxyphenyl)ethylamino]-3-(octanoylamino)-4-oxo-butane ate (1.4 g, 98.39% purity) as a yellow oil.

### Step 4

To a solution of tert-butyl (3R)-4-[2-(4-hydroxyphenyl)ethylamino]-3-(octanoylamino)-4-oxo-butanoate (1 g, 1.66 mmol, 72% purity, 1 eq) in DCM (10 mL) was added TFA (15.10 g, 132.42 mmol, 9.80 mL, 79.92 eq). The reaction mixture was stirred at 0° C. for 1 hr and then stirred at 25° C. for 1 hr. Desired mass was detected by LCMS. The reaction mixture was treated with toluene (35 mL*2) and concentrated in vacuum to remove solvent and TFA to get compound (3R)-4-[2-(4-hydroxyphenyl)ethylamino]-3-(octanoylamino)-4-oxo-butanoic acid (2 g, crude) as a white solid.

### Step 5

To a solution of MeNH₂ (2 M, 990.85 uL, 3 eq) in DMF (2.5 mL) was added (3R)-4-[2-(4-hydroxyphenyl)ethylamino]-3-(octanoylamino)-4-oxo-butanoic acid (250 mg, 660.57 umol, 1 eq), 1-hydroxybenzotriazole (116.03 mg, 858.74 umol, 1.3 eq) and 3-(ethyliminomethyleneamino)-N,N-dimethyl-propan-1-amine hydrochloride (164.62 mg, 858.74 umol, 1.3 eq) at 0° C. Then the mixture was allowed to warm to 25° C. The mixture was stirred for 3 hr. The reaction mixture was treated with water (80 mL) and the precipitate was filtered and concentrated in vacuum to get a residue. The residue was purified by prep-HPLC(column: Phenomenex Gemini-NX C18 75*30 mm*3 um; mobile phase: [water(10 mM NH4HCO3)-ACN]; B %: 15%-55%, 8 min) to give compound (2R)-N-[2-(4-hydroxyphenyl)ethyl]-N′-methyl-2-(octanoylamino)butanediamide (60 mg, 151.72 umol, 22.97% yield, 99% purity) as a white solid.

### Step 6

To a mixture of (3R)-4-[2-(4-hydroxyphenyl)ethylamino]-3-(octanoylamino)-4-oxo-butanoic acid (500 mg, 1.32 mmol, 1 eq), HATU (552.57 mg, 1.45 mmol, 1.1 eq), DIPEA (341.50 mg, 2.64 mmol, 460.24 uL, 2 eq) in DMF (5 mL) was added a mixture of hydroxylamine hydrochloride (279.95 mg, 2.64 mmol, 2 eq, HCl), DIPEA (341.50 mg, 2.64 mmol, 460.24 uL, 2 eq) and DMF (1.25 mL) in portions at 0° C. The resulting mixture was stirred at 25° C. for 12 hr. The reaction mixture was treated with water (10 mL), and extracted with EtOAc (20 mL*3), washed with brine (50 mL). And then dried with anhydrous Na₂SO₄, filtered and concentrated in vacuum. The reaction mixture was purified by prep-HPLC (column: Phenomenex luna C18 80*40 mm*3 um; mobile phase: [water(0.04% HCl)-ACN]; B %: 29%-45%, 7 min) to get compound N-[(1R)-3-(hydroxyamino)-1-[2-(4-hydroxyphenyl)ethylcarbamoyl]-3-oxo-propyl]octanamide (24 mg, 57.96 umol, 4.39% yield, 95.03% purity) as a white solid.

### Example 215: (R)-N′-(3-methoxyphenethyl)-2-octanamidosuccinamide

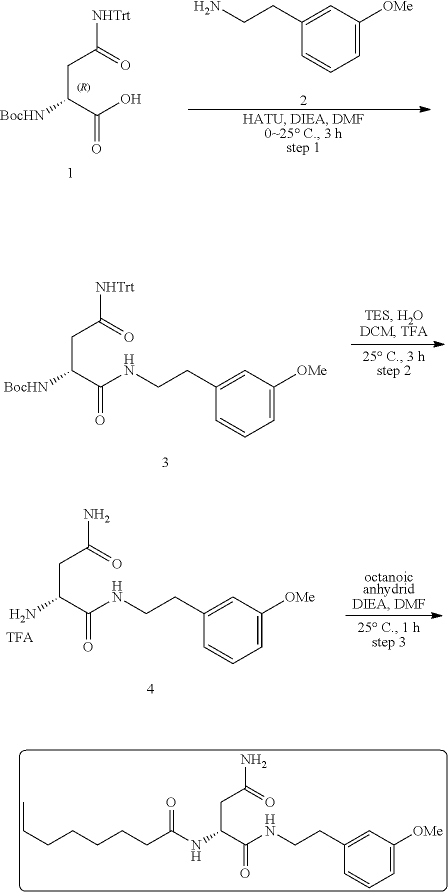

### Step 1

To a solution of (2R)-2-(tert-butoxycarbonylamino)-4-oxo-4-(tritylamino)butanoic acid (1 g, 2.11 mmol, 1 eq) and 2-(3-methoxyphenyl)ethanamine (382.36 mg, 2.53 mmol, 371.22 uL, 1.2 eq) in DMF (40 mL) was added DIPEA (599.16 mg, 4.64 mmol, 807.49 uL, 2.2 eq) and HATU (961.50 mg, 2.53 mmol, 1.2 eq) at 0° C. The mixture was stirred at 25° C. for 3 hr. The reaction mixture was treated with water (60 mL) and the precipitate was filtered, concentrated under reduced pressure to give compound tert-butyl N-[(1R)-1-[2-(3-methoxyphenyl)ethylcarbamoyl]-3-oxo-3-(tritylamino)propyl]carbamate (0.5 g, 780.77 umol, 37.05% yield, 94.9% purity) as a white solid.

### Step 2

The tert-butyl N-[(1R)-1-[2-(3-methoxyphenyl)ethylcarbamoyl]-3-oxo-3-(tritylamino)propyl]carbamate (0.5 g, 822.72 umol, 1 eq), TES (188.61 mg, 822.72 umol, 0.25 mL, 1 eq), H₂O (312.50 mg, 17.34 mmol, 312.50 uL, 21.08 eq), TFA (17.33 g, 151.95 mmol, 11.25 mL, 184.69 eq) and DCM (825.00 mg, 9.71 mmol, 625.00 uL, 11.81 eq) was stirred at 25° C. for 3 hr. LCMS showed the reaction was completed. The reaction mixture was concentrated to give compound (2R)-2-amino-N-[2-(3-methoxyphenyl)ethyl]butanediamide (0.7 g, crude, TFA) as a yellow solid.

### Step 3

To a solution of (2R)-2-amino-N-[2-(3-methoxyphenyl)ethyl]butanediamide (0.1 g, 263.62 umol, 1 eq, TFA) and octanoyl octanoate (106.93 mg, 395.43 umol, 1.5 eq) in DMF (1 mL) was added DIPEA (204.42 mg, 1.58 mmol, 275.50 uL, 6 eq). The mixture was stirred at 25° C. for 1 hr. Desired mass was detected by LCMS. The reaction mixture was concentrated under reduced pressure and purified by prep-HPLC (column: Phenomenex Gemini-NX C18 75*30 mm*3 um; mobile phase: [water(10 mM NH4HCO3)-ACN]; B %: 30%-50%, 8 min) to give compound (2R)-N-[2-(3-methoxyphenyl)ethyl]-2-(octanoylamino)butanediamide (67 mg, 169.42 umol, 64.27% yield, 99% purity) as a white solid.

### Example 216: (R)-N′-(3,4-dimethoxyphenethyl)-2-octanamidosuccinamide

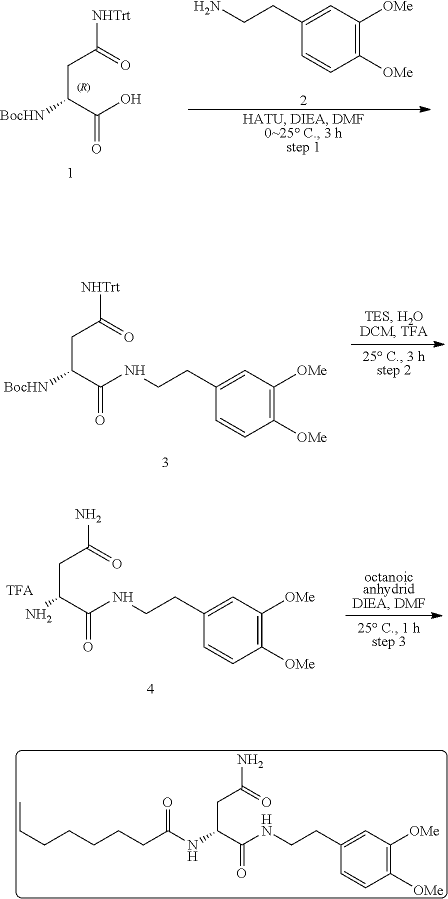

### Step 1

To a mixture of (2R)-2-(tert-butoxycarbonylamino)-4-oxo-4-(tritylamino)butanoic acid (500 mg, 1.05 mmol, 1 eq) and 2-(3,4-dimethoxyphenyl)ethanamine (228.35 mg, 1.26 mmol, 209.50 uL, 1.2 eq) in DMF (15 mL) was added HATU (479.09 mg, 1.26 mmol, 1.2 eq) and DIPEA (298.54 mg, 2.31 mmol, 402.35 uL, 2.2 eq) at 0° C. under N2. The mixture was stirred at 25° C. for 3 hours. The mixture was cooled to 0° C. and water (30 mL) was added slowly. The precipitate was filtered and the filter cake was washed by water (30 mL*2). The filter cake was concentrated in vacuum to get tert-butyl N-[(1R)-1-[2-(3,4-dimethoxyphenyl)ethylcarbamoyl]-3-oxo-3-(tritylamino)propyl]carbamate (600 mg, 799.67 umol, 76.16% yield, 85% purity) as a white solid.

### Step 2

A mixture of tert-butyl N-[(1R)-1-[2-(3,4-dimethoxyphenyl)ethylcarbamoyl]-3-oxo-3-(trityl amino)propyl]carbamate (0.5 g, 666.39 umol, 85% purity, 1 eq), TFA (13.86 g, 121.56 mmol, 9 mL, 182.41 eq), DCM (1.32 g, 15.54 mmol, 1 mL, 23.32 eq), H₂O (500.00 mg, 27.75 mmol, 0.5 mL, 41.64 eq) and triethylsilane (364.00 mg, 3.13 mmol, 0.5 mL, 4.70 eq) was stirred at 25° C. for 3 h. The mixture was concentrated in vacuum to get (2R)-2-amino-N-[2-(3,4-dimethoxy phenyl)ethyl]butanediamide (1 g, crude) as a white solid.

### Step 3

To a mixture of (2R)-2-amino-N-[2-(3,4-dimethoxyphenyl)ethyl]butanediamide (0.8 g, 586.29 umol, 30% purity, 1 eq, TFA) and octanoyl octanoate (190.24 mg, 703.54 umol, 1.2 eq) in DMF (20 mL) was added DIPEA (530.40 mg, 4.10 mmol, 714.83 uL, 7 eq) under N₂. The mixture was stirred at 25° C. for 3 hour. LCMS showed the reaction was completed and desired mass was detected. The mixture was concentrated in vacuum. The residue was purified by prep-HPLC (column: Waters Xbridge Prep OBD C18 150*40 mm*10 um; mobile phase: [water(10 mM NH4HCO3)-ACN]; B %: 20%-50%, 8 min) to give (2R)-N-[2-(3,4-dimethoxyphenyl)ethyl]-2-(octanoylamino)butanediamide (117 mg, 99% purity, 100% ee) as a white solid.

### Example 217: (R)-2-(3-cyclopentylpropanamido)-N′-phenethylsuccinamide

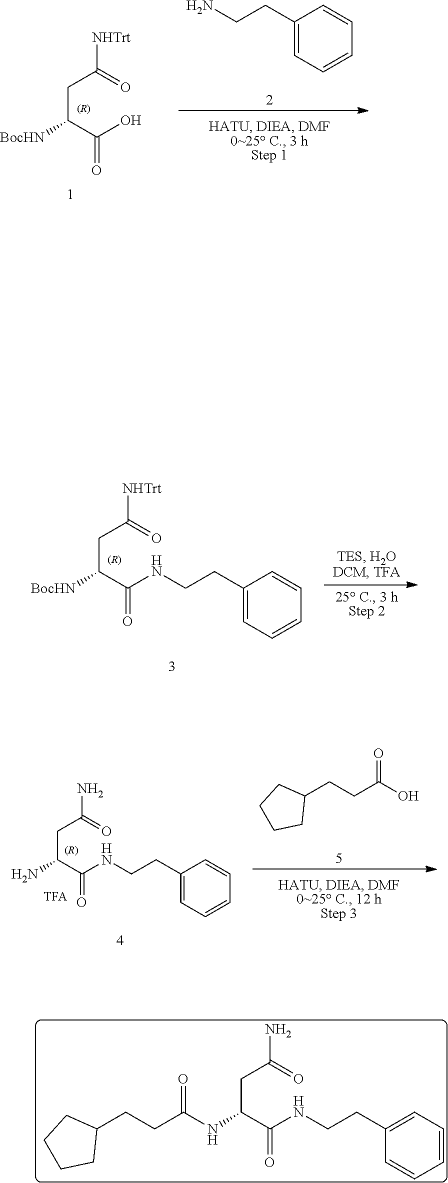

### Step 1

To a solution of (2R)-2-(tert-butoxycarbonylamino)-4-oxo-4-(tritylamino)butanoic acid (1 g, 2.11 mmol, 1 eq) and 2-phenylethanamine (306.43 mg, 2.53 mmol, 317.54 uL, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 3 hr. The reaction mixture was poured to water (20 ml), filtered and the filter cake was concentrated under reduced pressure to give the product tert-butyl N-[(1R)-3-oxo-1-(2-phenylethylcarbamoyl)-3-(tritylamino)propyl]carbamate (1 g, 1.73 mmol, 82.14% yield) as white solid.

### Step 2

A mixture of DCM (1.32 g, 15.54 mmol, 1 mL, 8.98 eq), TFA (27.72 g, 243.12 mmol, 18 mL, 140.45 eq), H₂O (500.00 mg, 27.75 mmol, 0.5 mL, 16.03 eq) and triethylsilane (364.00 mg, 3.13 mmol, 0.5 mL, 1.81 eq) was added tert-butyl N-[(1R)-3-oxo-1-(2-phenylethylcarbamoyl)-3-(tritylamino)propyl]carbamate (1 g, 1.73 mmol, 1 eq). The mixture was stirred at 25° C. for 3 hr under N2 atmosphere. The reaction mixture was concentrated under reduced pressure to give the crude product (2R)-2-amino-N-(2-phenylethyl)butanediamide (600 mg, crude, TFA) as white solid.

### Step 3

To a solution of 3-cyclopentylpropanoic acid (150 mg, 1.05 mmol, 150.60 uL, 1 eq) and (2R)-2-amino-N-(2-phenylethyl)butanediamide (500 mg, 1.43 mmol, 1.36 eq, TFA) in DMF (10 mL) was added HATU (481.32 mg, 1.27 mmol, 1.2 eq) and DIEA (409.01 mg, 3.16 mmol, 551.23 uL, 3 eq) at 0° C. The mixture was stirred at 25° C. for 12 hr. The reaction mixture was poured to H₂O (100 ml), filtered and the filter cake was concentrated under reduced pressure to give compound (2R)-2-(3-cyclopentylpropanoylamino)-N-(2-phenylethyl)butanediamide (72 mg, 184.28 umol, 17.47% yield, 100% purity) as yellow solid.

### Example 218: (R)-N′-(4-hydroxyphenethyl)-2-(4-(pyridin-4-yl)butanamido)succinamide

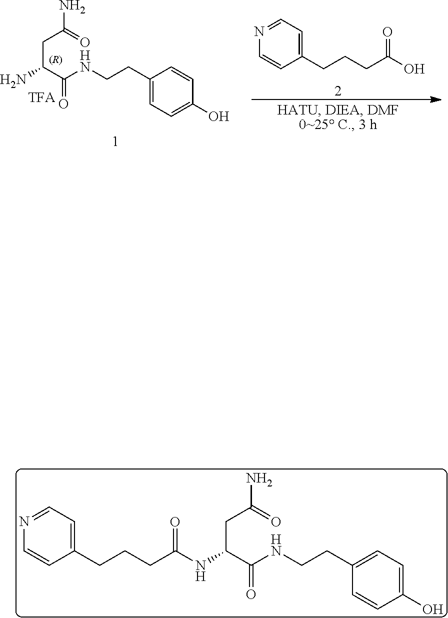

To a solution of 4-(4-pyridyl)butanoic acid (100 mg, 605.37 umol, 1 eq) and (2R)-2-amino-N-[2-(4-hydroxyphenyl)ethyl]butanediamide (300 mg, 821.23 umol, 1.36 eq, TFA) in DMF (5 mL) was added HATU (276.22 mg, 726.44 umol, 1.2 eq) and DIEA (172.13 mg, 1.33 mmol, 231.98 uL, 2.2 eq) at 0° C. The mixture was stirred at 25° C. for 3 hr. LC-MS showed the desired compound was detected. The reaction mixture was concentrated under reduced pressure to give a residue. The residue was purified by prep-HPLC ([water(10 mM NH₄HCO₃)-ACN]) to give compound (2R)-N-[2-(4-hydroxyphenyl)ethyl]-2-[4-(4-pyridyl)butanoylamino]butanedi amide (42 mg, 105.41 umol, 17.41% yield) as white solid.

### Example 219: (R)-N′-(4-hydroxyphenethyl)-2-(4-(piperidin-4-yl)butanamido)succinamide

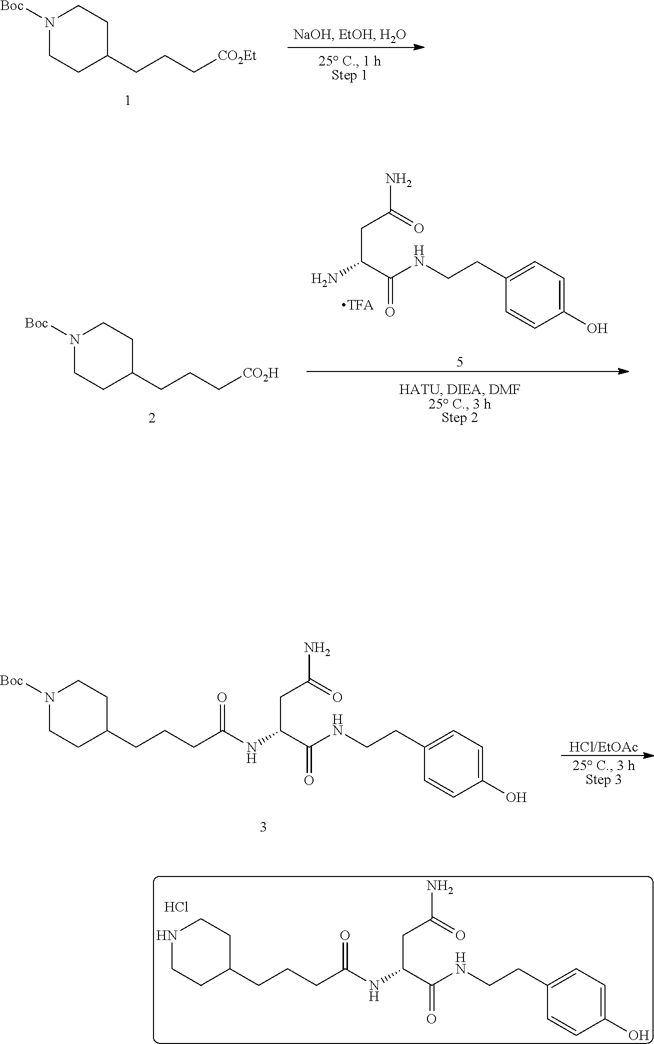

### Step 1

To a solution of tert-butyl 4-(4-ethoxy-4-oxo-butyl)piperidine-1-carboxylate (500 mg, 1.67 mmol, 1 eq) in EtOH (10 mL) and H₂O (4 mL) was added NaOH (200.38 mg, 5.01 mmol, 3 eq). The mixture was stirred at 25° C. for 1 hr. The reaction mixture was concentrated under reduced pressure to get a residue. The residue was adjusted pH to 3-4 with HCl (1 M) and extracted with EtOAc 200 mL (100 mL*2). The combined organic layers were dried over Na₂SO₄, filtered and concentrated under reduced pressure to give a residue. The residue was purified by column chromatography (SiO2, Petroleum ether/Ethyl acetate=10/1 to 0/1) to give compound 4-(1-tert-butoxycarbonyl-4-piperidyl)butanoic acid (250 mg, 626.49 umol, 37.52% yield, 68% purity) as colorless oil.

### Step 2

A mixture of 4-(1-tert-butoxycarbonyl-4-piperidyl)butanoic acid (200 mg, 737.05 umol, 1 eq), (2R)-2-amino-N-[2-(4-hydroxyphenyl)ethyl]butanediamide (269.25 mg, 737.05 umol, 1 eq, TFA), HATU (420.37 mg, 1.11 mmol, 1.5 eq) and DIPEA (285.78 mg, 2.21 mmol, 385.14 uL, 3 eq) in DMF (10 mL) was stirred at 25° C. for 3 hr under N₂ atmosphere. The reaction mixture was diluted with H₂O 100 mL and extracted with EtOAc 200 mL (100 mL*2). The combined organic layers were dried over Na₂SO₄, filtered and concentrated under reduced pressure to give a residue. The residue was purified by column chromatography (column: Welch Xtimate C18 100*25 mm*3 um; mobile phase:[water(0.05% HCl)-ACN]; B %: 20%-40%, 8 min) to give compound tert-butyl 4-[4-[[(1R)-3-amino-1-[2-(4-hydroxyphenyl)ethylcarbamoyl]-3-oxo-propyl]amino]-4-oxo-butyl]piperidine-1-carboxylate (150 mg, 277.22 umol, 37.61% yield) as colorless oil.

### Step 3

A solution of tert-butyl 4-[4-[[(1R)-3-amino-1-[2-(4-hydroxyphenyl)ethylcarbamoyl]-3-oxo-propyl]amino]-4-oxo-butyl]piperidine-1-carboxylate (50 mg, 92.41 umol, 1 eq) in HCl/EtOAc (5 mL) was stirred at 25° C. for 3 hr under N₂ atmosphere. The reaction mixture was concentrated under reduced pressure to give compound (2R)-N-[2-(4-hydroxyphenyl)ethyl]-2-[4-(4-piperidyl) butanoylamino]butanediamide (20 mg, 46.97 umol, 50.83% yield, 95% purity, HCl salt) as a yellow oil.

### Example 220: (R)-N′-(4-hydroxyphenethyl)-2-(4-(4-hydroxyphenyl)butanamido)-succinamide

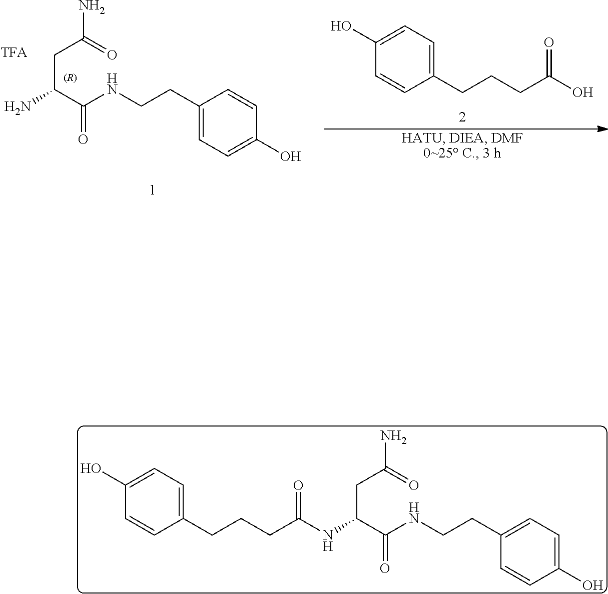

To a solution of 4-(4-hydroxyphenyl)butanoic acid (120 mg, 665.93 umol, 1 eq) and (2R)-2-amino-N-[2-(4-hydroxyphenyl)ethyl]butanediamide (300 mg, 821.23 umol, 1.23 eq, TFA) in DMF (5 mL) was added HATU (303.85 mg, 799.11 umol, 1.2 eq) and DIEA (189.35 mg, 1.47 mmol, 255.18 uL, 2.2 eq) at 0° C. The mixture was stirred at 25° C. for 3 hr. LC-MS showed the desired compound was detected. The reaction mixture was concentrated under reduced pressure to give a residue. The residue was purified by prep-HPLC ([water (10 mM NH₄HCO₃)-ACN]) to give compound2R)-2-[4-(4-hydroxyphenyl)butanoylamino]-N-[2-(4-hydroxyphenyl)ethyl]butanediamide (152 mg, 367.62 umol, 55.20% yield) as white solid.

### Example 221: (R)-2-octanamido-N′-(2-phenoxyethyl)succinamide

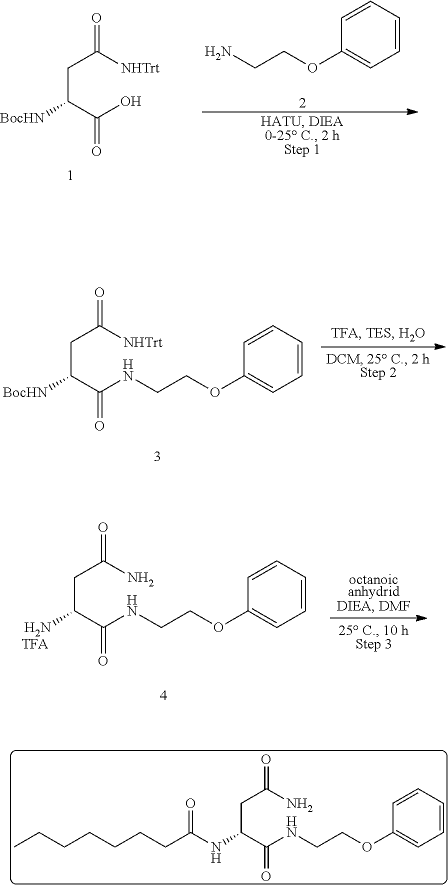

### Step 1

To a solution of (2R)-2-(tert-butoxycarbonylamino)-4-oxo-4-(tritylamino)butanoic acid (1 g, 2.11 mmol, 1 eq) and 2-phenoxyethanamine (346.89 mg, 2.53 mmol, 330.37 uL, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 2 hr. LC-MS showed the desired compound was detected. The reaction mixture was pour to water (20 ml), filtered and the filter cake was concentrated under reduced pressure to give the product tert-butyl N-[(1R)-3-oxo-1-(2-phenoxyethylcarbamoyl)-3-(tritylamino)propyl]carbamate (1 g, 1.62 mmol, 76.73% yield, 96% purity) as white solid.

### Step 2

A mixture of TFA (27.72 g, 243.11 mmol, 18 mL, 160.37 eq), DCM (1.32 g, 15.54 mmol, 1 mL, 10.25 eq), triethylsilane (364.00 mg, 3.13 mmol, 0.5 mL, 2.07 eq) and H₂O (500.00 mg, 27.75 mmol, 0.5 mL, 18.31 eq) was added tert-butyl N-[(1R)-3-oxo-1-(2-phenoxyethylcarbamoyl)-3-(tritylamino)propyl]carbamate (900 mg, 1.52 mmol, 1 eq), then the mixture was stirred at 25° C. for 2 hr under N₂ atmosphere. The reaction mixture was concentrated under reduced pressure to give a residue. The crude product (2R)-2-amino-N-(2-phenoxyethyl)butanediamide (500 mg, crude, TFA) as white solid.

### Step 3

To a solution of (2R)-2-amino-N-(2-phenoxyethyl)butanediamide (500 mg, 1.37 mmol, 1 eq, TFA) in DMF (10 mL) was added DIEA (530.69 mg, 4.11 mmol, 715.22 uL, 3 eq) and octanoyl octanoate (444.14 mg, 1.64 mmol, 1.2 eq). The mixture was stirred at 25° C. for 10 hr. LC-MS showed the desired compound was detected. The reaction mixture was filtered and the filter cake was concentrated under reduced pressure to give a residue. The residue was purified by prep-HPLC ([water (10 mM NH₄HCO₃)-ACN]) to give compound (2R)-2-(octanoylamino)-N-(2-phenoxyethyl)butanediamide (120 mg, 305.18 umol, 22.30% yield, 96% purity) as white solid.

### Example 222: (R)-N′-(2-(2,6-difluorophenoxy)ethyl)-2-octanamidosuccinamide

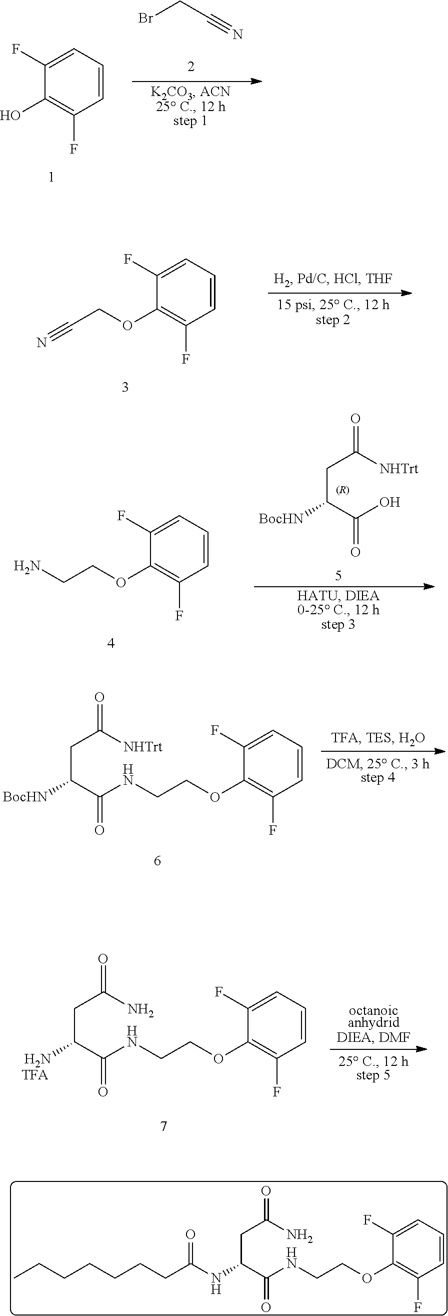

### Step 1

To a mixture of 2,6-difluorophenol (8 g, 61.49 mmol, 1 eq), 2-bromoacetonitrile (8.85 g, 73.79 mmol, 4.92 mL, 1.2 eq) in ACN (50 mL) was added K₂CO₃ (17.00 g, 122.99 mmol, 2 eq) and was stirred at 25° C. for 12 hr under N2 atmosphere. The combined organic phase was diluted with EtOAc 100 mL and washed with water 300 mL (100 mL*3) and brine 120 mL (60 mL*2), dried with anhydrous Na₂SO₄, filtered and concentrated under reduced pressure to give compound 2-(2,6-difluorophenoxy)acetonitrile (4 g, 23.65 mmol, 38.46% yield) as brown solid without purification.

### Step 2

To a solution of methyl 2-(2,6-difluorophenoxy)acetonitrile (0.4 g, 2.37 mmol, 1 eq) in THF (10 mL) was added HCl (12 M, 394.18 uL, 2 eq), Pd/C (0.03 g, 39.99 mmol, 10% purity, 16.91 eq) under N₂. The suspension was degassed under vacuum and purged with H₂ several times. The mixture was stirred under H₂ (15 psi) at 25° C. for 12 hr. The mixture is filtered through celite and the solvent is removed under reduced pressure to give compound 2-(2,6-difluorophenoxy) ethanamine (0.3 g, 1.43 mmol, 60.51% yield, HCl) as white solid.

### Step 3

To a solution of (2R)-2-(tert-butoxycarbonylamino)-4-oxo-4-(tritylamino)butanoic acid (456 mg, 960.92 umol, 1 eq) and 2-(2,6-difluorophenoxy)ethanamine (241.71 mg, 1.15 mmol, 361.03 uL, 1.2 eq, HCl) in DMF (10 mL) was added HATU (438.44 mg, 1.15 mmol, 1.2 eq) and DIEA (273.22 mg, 2.11 mmol, 368.21 uL, 2.2 eq) at 0° C. The mixture was stirred at 25° C. for 12 hr. The reaction mixture was quenched by addition H₂O 60 mL and extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=10/1 to 2/1) to give compound tert-butyl N-[(1R)-1-[2-(2,6-difluorophenoxy)ethylcarbamoyl]-3-oxo-3-(tritylamino)propyl]carbamate (0.3 g, 457.37 umol, 47.60% yield, 96% purity) as white solid.

### Step 4

The tert-butyl N-[(1R)-1-[2-(2,6-difluorophenoxy)ethylcarbamoyl]-3-oxo-3-(tritylamino) propyl]carbamate (0.4 g, 635.23 umol, 1 eq) in TES (364.07 mg, 1.59 mmol, 0.5 mL, 2.5 eq), H₂O (500.00 mg, 27.75 mmol, 0.5 mL, 43.68 eq), DCM (1.32 g, 15.54 mmol, 1.00 mL, 24.47 eq) and TFA (27.72 g, 243.12 mmol, 18.00 mL, 382.72 eq) was stirred at 25° C. for 3 hr under N₂ atmosphere. The reaction mixture was concentrated under reduced pressure to give compound (2R)-2-amino-N-[2-(2,6-difluorophenoxy)ethyl]butanediamide (0.4 g, crude, TFA) as white solid.

### Step 5

To a mixture of (2R)-2-amino-N-[2-(2,6-difluorophenoxy)ethyl]butanediamide (0.4 g, 996.80 umol, 1 eq, TFA) in DMF (5 mL) was added DIEA (386.48 mg, 2.99 mmol, 520.86 uL, 3 eq), octanoyl octanoate (404.31 mg, 1.50 mmol, 1.5 eq). The mixture was degassed and purged with N₂ for 3 times. The mixture was stirred at 25° C. for 12 hr under N₂ atmosphere. The reaction mixture was concentrated under reduced pressure to get a residue. The residue was purified by prep-HPLC (column: Phenomenex luna C18 250*50 mm*10 um; mobile phase: [water(0.1% TFA)-ACN]; B %: 30%-60%, 10 min) to give compound (2R)-N-[2-(2,6-difluorophenoxy)ethyl]-2-(octanoylamino) butanediamide (166 mg, 401.49 umol, 40.28% yield, 100% purity) as white solid.

### Example 223: (R)-N′-(3,5-difluoro-4-hydroxyphenethyl)-2-octanamidosuccinamide

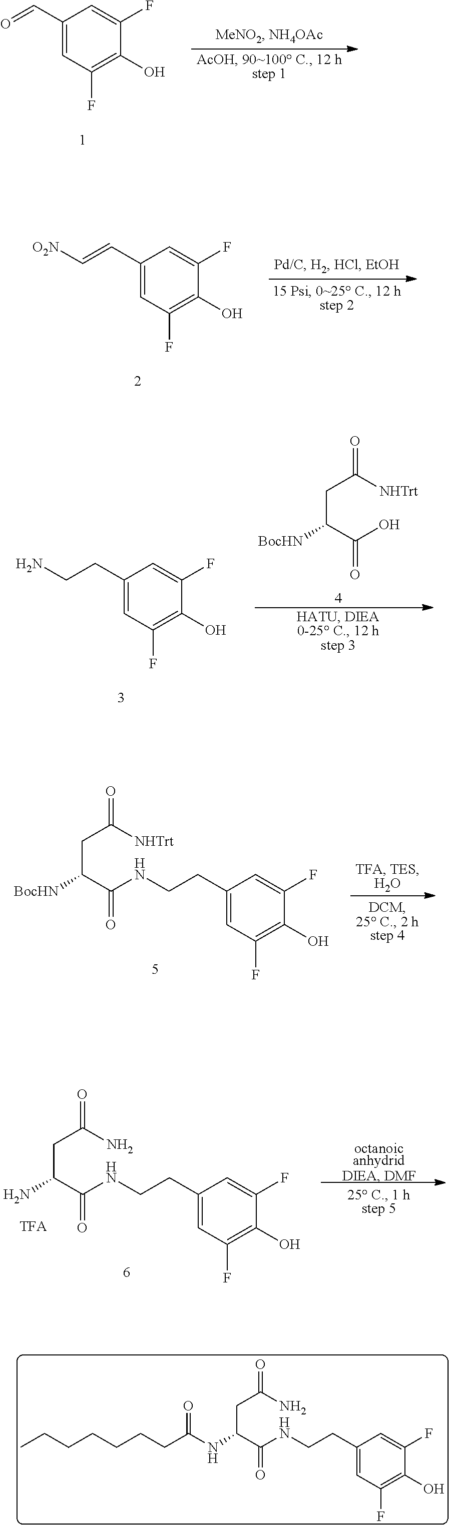

### Step 1

To a solution of NH₄OAc (3.05 g, 39.53 mmol, 2.5 eq) in AcOH (70 mL) and nitromethane (27.84 g, 456.10 mmol, 24.64 mL, 28.84 eq) was added 3,5-difluoro-4-hydroxy-benzaldehyde (2.5 g, 15.81 mmol, 1 eq) at 90° C., and stirred at 100° C. for 12 h. TLC showed the starting reactant consumed. The reaction mixture was cooled to 15° C., quenched with H₂O (200 mL) and sat·NaHCO₃ (500 mL). The mixture was extracted with ethyl acetate (100 mL*3), and the organic phase was dried with Na₂SO₄, filtered and the filtrate was concentrated in vacuo to 2,6-difluoro-4-[(E)-2-nitrovinyl]phenol (3 g, crude) as yellow oil.

### Step 2

To a solution of Pd/C (400 mg, 10% purity) and HCl (12 M, 3.11 mL, 2.5 eq) in EtOH (40 mL) was added 2,6-difluoro-4-[(E)-2-nitrovinyl]phenol (3 g, 14.92 mmol, 1 eq) at 0° C., stirred at 25° C. for 12 h under H₂ under 15 psi. LCMS showed the starting reactant consumed. The mixture was filtered and concentrated the filtrate under reduced pressure. The crude product was triturated with EtOAc (10 mL) at 25° C. for 15 min and then filtered, the solid was collected and concentrated under reduced pressure to give 4-(2-aminoethyl)-2,6-difluoro-phenol (1 g, crude, HCl) as a gray white solid.

### Step 3

To a solution of (2R)-2-(tert-butoxycarbonylamino)-4-oxo-4-(tritylamino)butanoic acid (300 mg, 632.18 umol, 1 eq) and 4-(2-aminoethyl)-2,6-difluoro-phenol (198.78 mg, 948.27 umol, 1.5 eq, HCl) in DMF (5 mL) was added HATU (288.45 mg, 758.62 umol, 1.2 eq) and DIPEA (245.11 mg, 1.90 mmol, 330.34 uL, 3 eq) at 0° C. The mixture was stirred at 25° C. for 12 h. TLC showed the starting reactant consumed. The mixture was poured into H₂O (10 mL), then extracted with EtOAc (10 mL*3), combined organic layer was washed with brine (10 mL*2), dried over Na₂SO₄, filtered and concentrated the filtrate under reduced pressure. The residue was purified by column chromatography (SiO₂, Petroleum ether/Ethyl acetate=10 to 3/1) to give the product tert-butyl N-[(1R)-1-[2-(3,5-difluoro-4-hydroxy-phenyl)ethylcarbamoyl]-3-oxo-3-(tritylamino)propyl]carbamate (300 mg, 476.42 umol, 75.36% yield, —purity) as a brown solid.

### Step 4

The tert-butyl N-[(1R)-1-[2-(3,5-difluoro-4-hydroxy-phenyl)ethylcarbamoyl]-3-oxo-3-(tritylamino) propyl]carbamate (300 mg, 476.42 umol, 1 eq) was dissolved with TES (0.5 mL), H₂O (0.5 mL), DCM (1 mL) and TFA (18 mL), stirred at 25° C. for 2 h. LCMS showed the starting reactant was consumed. The mixture was concentrated under reduced pressure to give (2R)-2-amino-N-[2-(3,5-difluoro-4-hydroxy-phenyl)ethyl]butanediamide (190 mg, crude, TFA) as yellow oil.

### Step 5

To a solution of octanoyl octanoate (140.83 mg, 520.83 umol, 1.1 eq) and (2R)-2-amino-N-[2-(3,5-difluoro-4-hydroxy-phenyl)ethyl]butanediamide (190 mg, 473.48 umol, 1 eq, TFA) in DMF (3 mL) was added DIPEA (183.58 mg, 1.42 mmol, 247.41 uL, 3 eq). The mixture was stirred at 25° C. for 1 hr. LCMS showed the starting reactant was consumed. The mixture was concentrated under reduced pressure to give a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 20%-40%, 8 min) to give compound (2R)-N-[2-(3,5-difluoro-4-hydroxy-phenyl)ethyl]-2-(octanoylamino)butanediamide (140 mg, 335.22 umol, 70.80% yield, 99% purity) as a white solid.

### Example 224: (R)-N-(4-amino-1-(1-methyl-1H-imidazol-2-yl)-1,4-dioxobutan-2-yl)octanamide

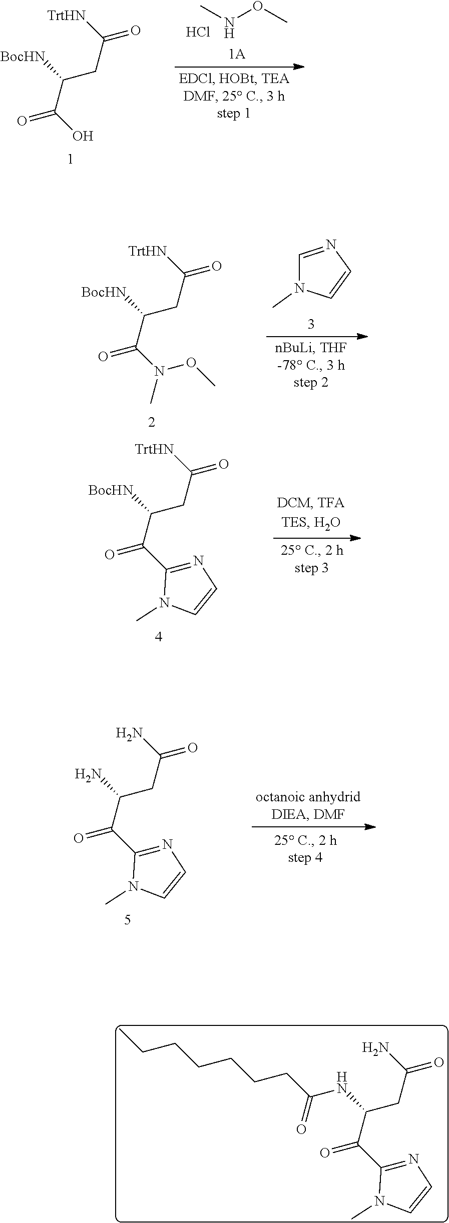

### Step 1

To a solution of (2R)-2-(tert-butoxycarbonylamino)-4-oxo-4-(tritylamino)butanoic acid (2 g, 4.21 mmol, 1 eq) and N-methoxymethanamine (308.92 mg, 5.06 mmol, 1.2 eq) in DMF (20 mL) was added EDCI (969.51 mg, 5.06 mmol, 1.2 eq) and HOBt (683.36 mg, 5.06 mmol, 1.2 eq) and TEA (1.28 g, 12.64 mmol, 1.76 mL, 3 eq). The mixture was stirred at 25° C. for 3 hr. The reaction mixture was concentrated under reduced pressure to remove solvent. The residue was diluted with H₂O 30 mL and extracted with EtOAc 40 mL (20 mL* 2). The combined organic layers were washed with brine 20 mL (10 mL*2), dried over, filtered and concentrated under reduced pressure to give a residue. The residue was purified by column chromatography (SiO₂, Petroleum ether/Ethyl acetate=1/0 to 0/1) to give compound tert-butyl N-[(1R)-1-[methoxy(methyl)carbamoyl]-3-oxo-3-(tritylamino)propyl]carbamate (0.73 g, 1.41 mmol, 66.36% yield) as white solid.

### Step 2

To a solution of 1-methylimidazole (396.55 mg, 4.83 mmol, 385.00 uL, 5 eq) in THF (35 mL) was added dropwise n-BuLi (2.5 M, 2.82 mL, 7.3 eq) at −78° C. After addition, the mixture was stirred at this temperature for 30 min. The tert-butyl N-[(1R)-1-[methoxy(methyl)carbamoyl]-3-oxo-3-(tritylamino)propyl]carbamate (0.5 g, 965.97 umol, 1 eq) in THF (35 mL) was added dropwise at −78° C. The resulting mixture was stirred at −78° C. for 2.5 hr. The reaction mixture was quenched by addition sat·NH₄Cl 50 mL at 0° C., diluted with H₂O 10 mL and extracted with EtOAc 60 mL (30 mL*2). The combined organic layers were dried over Na₂SO₄, filtered and concentrated under reduced pressure to give a residue. The residue was purified by column chromatography (SiO₂, Petroleum ether/Ethyl acetate=1/0 to 0/1) to give compound tert-butyl N-[(1R)-1-(1-methylimidazole-2-carbonyl)-3-oxo-3-(tritylamino)propyl]carbamate (0.22 g, 408.44 umol, 42.28% yield) as white solid.

### Step 3

A mixture of tert-butyl N-[(1R)-1-(1-methylimidazole-2-carbonyl)-3-oxo-3-(tritylamino)propyl]carbamate (0.2 g, 371.31 umol, 1 eq), TFA (9 mL), DCM (0.5 mL), H₂O (0.25 mL) and TES (0.25 mL) was stirred at 25° C. for 2 hr under N2 atmosphere. The reaction mixture was concentrated under reduced pressure to give compound (3R)-3-amino-4-(1-methylimidazol-2-yl)-4-oxo-butanamide (0.2 g, crude) as colorless oil.

### Step 4

A mixture of (3R)-3-amino-4-(1-methylimidazol-2-yl)-4-oxo-butanamide (0.2 g, 644.69 umol, 1 eq, TFA), octanoyl octanoate (174.33 mg, 644.69 umol, 1 eq) and DIPEA (416.61 mg, 3.22 mmol, 561.46 uL, 5 eq) in DMF (10 mL) was stirred at 25° C. for 2 hr under N₂ atmosphere. The reaction mixture was concentrated under reduced pressure to get residue. The residue was purified by prep-HPLC (column: Welch Xtimate C18 100*25 mm*3 um; mobile phase: [water(0.05% HCl)-ACN]; B %: 10%-30%, 8 min) and prep-TLC (SiO2, DCM: MeOH=10:1) to give compound N-[(1R)-3-amino-1-(1-methylimidazole-2-carbonyl)-3-oxo-propyl]octanamide (6 mg, 12.66 umol, 2.72% yield, 68% purity) as white solid.

### Example 225: (R)-N-(4-amino-1-(1-benzyl-1H-imidazol-2-yl)-1,4-dioxobutan-2-yl)octanamide

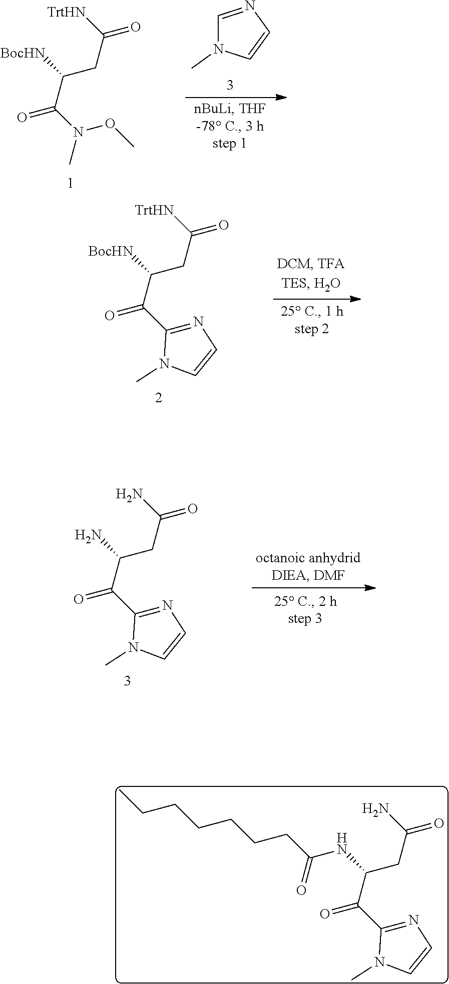

### Step 1

To a solution of 1-benzylimidazole (458.45 mg, 2.90 mmol, 5 eq) in THF (10 ml) was added dropwise n-BuLi (2.5 M, 1.69 mL, 7.3 eq) at −78° C. The mixture was stirred at this temperature for 30 min, and then tert-butyl N-[(1R)-1-[methoxy(methyl)carbamoyl]-3-oxo-3-(tritylamino)propyl]carbamate (0.3 g, 579.58 umol, 1 eq) in THF (5 ml) was added dropwise at −78° C. The resulting mixture was stirred at −78° C. for 2.5 hr. The reaction mixture was quenched by addition sat·NH₄Cl 50 mL at 0° C., and then diluted with H₂O 10 mL and extracted with EtOAc 60 mL (30 mL*2). The combined organic layers were dried over Na₂SO₄, filtered and concentrated under reduced pressure to give a residue. The residue was purified by column chromatography (SiO₂, Petroleum ether/Ethyl acetate=1/0 to 0/1) to give compound tert-butyl N-[(1R)-1-(1-benzylimidazole-2-carbonyl)-3-oxo-3-(tritylamino)propyl]carbamate (0.18 g, crude) as white solid.

### Step 2

A mixture of tert-butyl N-[(1R)-1-(1-benzylimidazole-2-carbonyl)-3-oxo-3-(tritylamino)propyl]carbamate (160 mg, 260.28 umol, 1 eq), TFA (3.6 mL), DCM (0.2 mL), H₂O (0.1 mL) and TES (0.1 mL) was stirred at 25° C. for 1 hr under N₂ atmosphere. The reaction mixture was concentrated under reduced pressure to give compound (3R)-3-amino-4-(1-benzylimidazol-2-yl)-4-oxo-butanamide (160 mg, crude) as colorless oil.

### Step 3

A mixture of (3R)-3-amino-4-(1-benzylimidazol-2-yl)-4-oxo-butanamide (160 mg, 518.20 umol, 1 eq, HCl), octanoyl octanoate (140.12 mg, 518.20 umol, 1 eq), DIPEA (334.87 mg, 2.59 mmol, 451.30 uL, 5 eq) in DMF (2 mL) was stirred at 25° C. for 1 hr under N₂ atmosphere. The reaction mixture was concentrated under reduced pressure to get a residue. The residue was purified by prep-HPLC (column: Phenomenex Gemini NX-C18 (75*30 mm*3 um); mobile phase: [water(0.05% HCl)-ACN]; B %: 20%-30%, 8 min) to give compound N-[(1R)-3-amino-1-(1-benzylimidazole-2-carbonyl)-3-oxo-propyl]octanamide (7 mg, 15.81 umol, 18.00% yield, 90% purity) as white solid.

### Example 226: (R)-2-hexanamido-N1-(4-(trifluoromethyl)phenethyl)succinamide

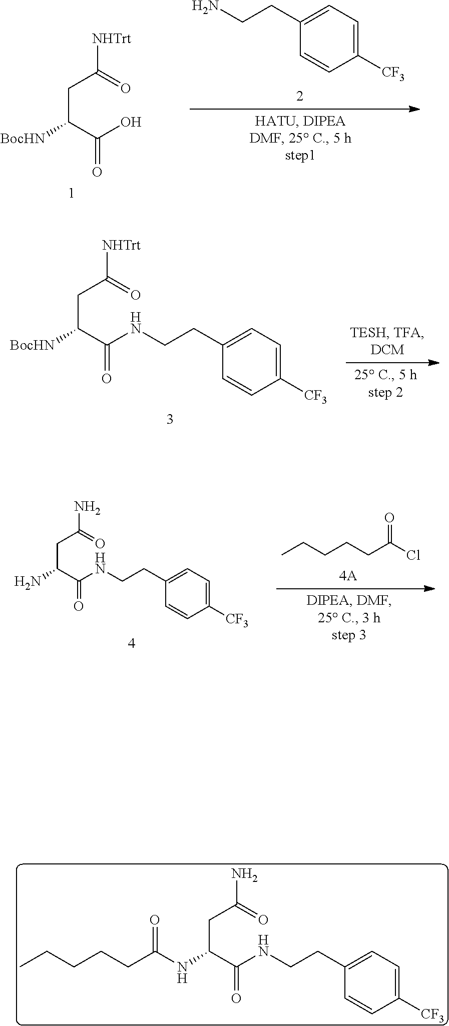

### Step 1

A solution of (2R)-2-(tert-butoxycarbonylamino)-4-oxo-4-(tritylamino)butanoic acid (500 mg, 1.05 mmol, 1 eq) and 2-[4-(trifluoromethyl)phenyl]ethanamine (50.03 mg, 264.46 umol, 2.51e-1 eq) in DMF (20 mL) was added HATU (600.94 mg, 1.58 mmol, 1.5 eq) and DIPEA (272.35 mg, 2.11 mmol, 367.05 uL, 2 eq) at 25° C. The mixture was stirred at 25° C. for 5 hr under N₂ atmosphere. The reaction mixture was diluted with H₂O 100 mL and extracted with EtOAc 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=10/1 to 0/1) to give the compound tert-butyl N-[(1R)-3-oxo-1-[2-[4-(trifluoromethyl)phenyl]ethylcarbamoyl]-3-(tritylamino)propyl]carbamate (300 mg, 362.39 umol, 34.39% yield, 78% purity) as white solid.

### Step 2

A mixture of tert-butyl N-[(1R)-3-oxo-1-[2-[4-(trifluoromethyl)phenyl]ethylcarbamoyl]-3-(tritylamino)propyl]carbamate (200 mg, 309.74 umol, 1 eq), DCM (1 mL), TESH (0.5 mL) H₂O (0.5 mL) and TFA (10 mL) was stirred at 25° C. for 5 hr under N₂ atmosphere. The reaction mixture was concentrated under reduced pressure to give compound (2R)-2-amino-N-[2-[4-(trifluoromethyl)phenyl]ethyl]butanediamide (100 mg, crude, TFA) as white solid.

### Step 3

A mixture of (2R)-2-amino-N-[2-[4-(trifluoromethyl)phenyl]ethyl]butanediamide (100 mg, 239.63 umol, 1 eq, TFA), hexanoyl chloride (48.38 mg, 359.45 umol, 50.19 uL, 1.5 eq), DIPEA (92.91 mg, 718.90 umol, 125.22 uL, 3 eq) in DMF (5 mL) was stirred at 25° C. for 3 hr under N₂ atmosphere. The reaction mixture was diluted with H₂O 100 mL and extracted with EtOAc 200 mL (100 mL*2). The combined organic layers were dried over Na₂SO₄, filtered and concentrated under reduced pressure to give a residue. The residue was purified by prep-HPLC (column: Welch Xtimate C18 100*25 mm*3 um; mobile phase: [water(0.05% HCl)-ACN]; B %: 20%-40%, 8 min) to give compound (2R)-2-(hexanoylamino)-N-[2-[4-(trifluoromethyl)phenyl]ethyl]butanediamide (40 mg, 99.65 umol, 69.30% yield) as white solid.

### Example 227: (R,E)-2-(4-cyclopentylbut-2-enamido)-N′-(4-hydroxyphenethyl)succinamide and (R)-2-(4-cyclopentylbutanamido)-N′-(4-hydroxyphenethyl)succinamide

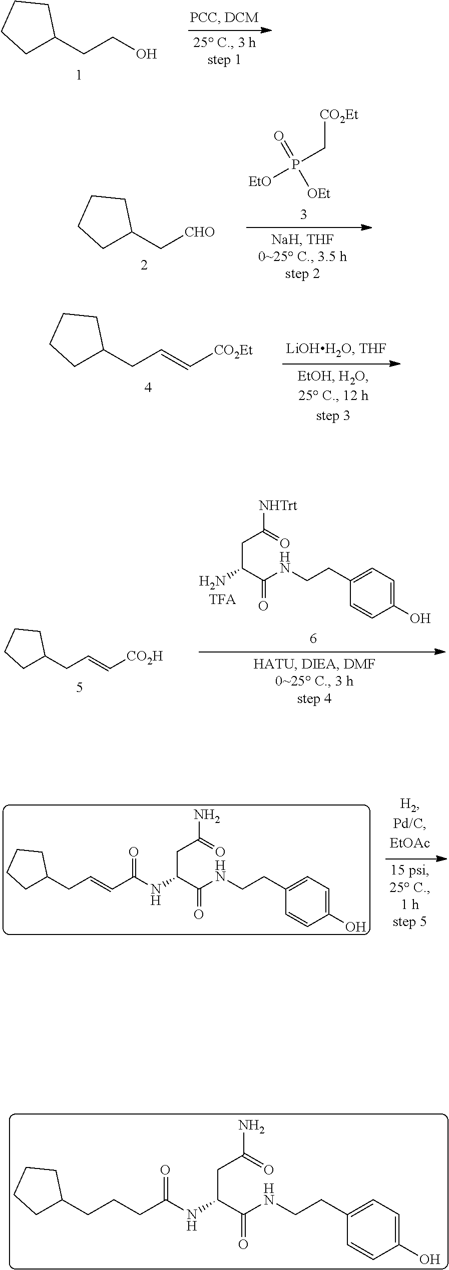

### Step 1

To a solution of 2-cyclopentylethanol (2 g, 17.52 mmol, 2.17 mL, 1 eq) in DCM (500 mL) was added PCC (5.66 g, 26.27 mmol, 1.5 eq) and stirred at 25° C. for 3 h. TLC showed the reaction was completed. The reaction mixture was diluted with MTBE (300 mL) stirred at 25° C. for 1 h, and then filtered through a pad of celite and silica gel (1:1). The filtrate was carefully concentrated to give compound 2-cyclopentylacetaldehyde (2 g, crude) was obtained as a yellow oil.

### Step 2

To a mixture of NaH (927.08 mg, 23.18 mmol, 60% purity, 1.3 eq) in THF (50 mL) was added ethyl 2-diethoxyphosphorylacetate (4.20 g, 18.72 mmol, 3.71 mL, 1.05 eq) dropwise at 0° C. under N₂. The mixture was stirred at 0° C. for 5 min. A solution of 2-cyclopentylacetaldehyde (2 g, 17.83 mmol, 1 eq) in THF (20 mL) was added dropwise at 0° C. The mixture was stirred at 0° C. for 25 min and then warmed to 25° C. stirred for 3 hr. TLC showed the reaction was completed and desired mass was detected by LCMS. The mixture was quenched by sat. NH₄Cl (50 mL) and the mixture was extracted with ethyl acetate (50 mL*2). The combined organic phase was washed with brine (50 mL), dried with anhydrous Na₂SO₄, filtered and concentrated in vacuum to get ethyl 4-cyclopentylbut-2-enoate (3.3 g, crude) as colorless oil.

### Step 3

To a mixture of ethyl 4-cyclopentylbut-2-enoate (2.5 g, 13.72 mmol, 1 eq) in THF (6 mL), EtOH (6 mL) and H₂O (6 mL) was added LiOH·H₂O (2.88 g, 68.58 mmol, 5 eq) in one portion at 25° C. The mixture was stirred at 25° C. for 12 hours. TLC showed the reaction was completed. To the mixture was added water (20 mL) and the mixture was extracted with ethyl acetate (20 mL). Then the aqueous phase treated with 4N HCl (20 mL) to pH=2-3. The aqueous phase was extracted with ethyl acetate (20 mL*4). The combined organic phase was washed with brine (50 mL), dried with anhydrous Na₂SO₄, filtered and concentrated in vacuum to get 4-cyclopentylbut-2-enoic acid (1 g, 6.48 mmol, 47.28% yield) as a colorless oil.

### Step 4

To a mixture of (2R)-2-amino-N-[2-(4-hydroxyphenyl)ethyl]butanediamide (500 mg, 944.42 umol, 69% purity, 1 eq, TFA) and 4-cyclopentylbut-2-enoic acid (150 mg, 944.42 umol, 203.51 uL, 97% purity, 1 eq) in DMF (15 mL) was added DIPEA (854.40 mg, 6.61 mmol, 1.15 mL, 7 eq) and HATU (430.92 mg, 1.13 mmol, 1.2 eq) at 0° C. under N₂. The mixture was stirred at 25° C. for 3 hour. LCMS showed the reaction was completed and desired mass was detected. The mixture was cooled to 0° C. and water (30 mL) was added slowly, then the precipitate was filtered and the filter cake was washed by water (30 mL). The filter cake was concentrated in vacuum to get crude product as a yellow solid. The crude product was purified by p-HPLC (column: Waters Xbridge BEH C18 100*30 mm*10 um; mobile phase: [water(10 mM NH4HCO3)-ACN]; B %: 20%-40%, 8 min) to get (2R)-2-(4-cyclopentylbut-2-enoylamino)-N-[2-(4-hydroxyphenyl)ethyl]butanediamide (12 mg, 30.66 umol, 3.25% yield, 99% purity) as a white solid.

### Step 5

To a solution of (2R)-2-(4-cyclopentylbut-2-enoylamino)-N-[2-(4-hydroxyphenyl)ethyl]butane diamide (150 mg, 162.59 umol, 1.09 mL, 42% purity, 1 eq) in EtOAc (5 mL) was added Pd/C (0.1 g, 10% purity) under Ar. Then the mixture was degassed under vacuum and purged with H₂ three times. The mixture was stirred under H₂ (15 psi) at 25° C. for 1 hour. LCMS showed the reaction was completed and desired mass was detected. The reaction mixture was filtered through a pad of celite and the filtrate was concentrated in vacuum. The residue was purified by p-HPLC (column: Waters Xbridge BEH C18 100*30 mm*10 um; mobile phase: [water(10 mM NH₄HCO₃)-ACN]; B %: 20%-40%, 8 min) to give (2R)-2-(4-cyclopentylbutanoylamino)-N-[2-(4-hydroxyphenyl)ethyl]butanediamide (24 mg, 59.72 umol, 36.73% yield, 96.91% purity) as a white solid.

### Example 228: (R,E)-2-(4-cyclopentylbut-2-enamido)-N′-phenethylsuccinamide and (R)-2-(4-cyclopentylbutanamido)-N′-phenethylsuccinamide

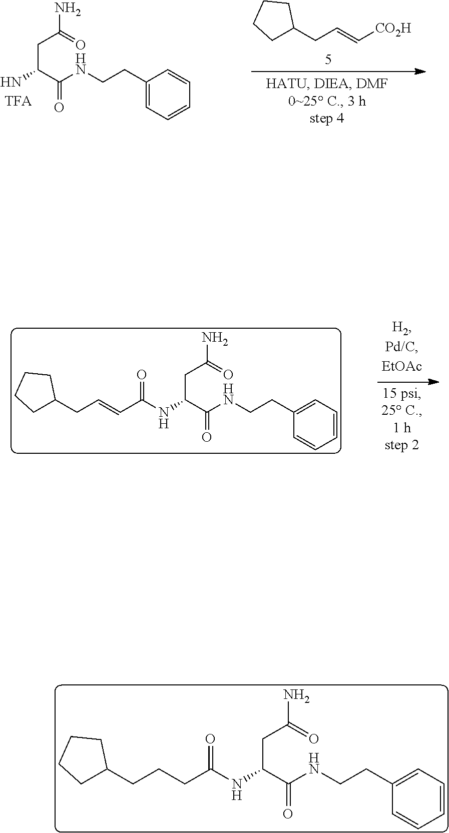 [SMILES: NC(=O)C[C@@H](N)C(=O)NCCc1ccccc1 ; O=C(O)/C=C/CC1CCCC1 ; NC(=O)C[C@@H](NC(=O)/C=C/CC1CCCC1)C(=O)NCCc1ccccc1 ; NC(=O)C[C@@H](NC(=O)CCCC1CCCC1)C(=O)NCCc1ccccc1]

### Step 1

To a mixture of (2R)-2-amino-N-(2-phenylethyl)butanediamide (300 mg, 772.96 umol, 90% purity, 1 eq, TFA) and 4-cyclopentylbut-2-enoic acid (150 mg, 943.54 umol, 203.51 uL, 97% purity, 1.22 eq) in DMF (3 mL) was added DIPEA (699.29 mg, 5.41 mmol, 942.43 uL, 7 eq) and HATU (352.69 mg, 927.56 umol, 1.2 eq) at 0° C. under N₂. The mixture was stirred at 25° C. for 3 hour. LCMS showed the reaction was completed and desired mass was detected. The mixture was cooled to 0° C. and was added water (30 mL) slowly, then the precipitate was filtered and the filter cake was washed by water (30 mL). The filter cake was concentrated in vacuum to get a crude product. The crude product was purified by p-HPLC (column: Waters Xbridge BEH C18 100*30 mm*10 um; mobile phase: [water(10 mM NH4HCO3)-ACN]; B %: 25%-55%, 8 min) to give (2R)-2-(4-cyclopentylbut-2-enoylamino)-N-(2-phenylethyl)butanediamide (22 mg, 58.63 umol, 7.59% yield, 99% purity) as a white solid.

### Step 2

To a solution of (2R)-2-(4-cyclopentylbut-2-enoylamino)-N-(2-phenylethyl)butanediamide (100 mg, 220.74 umol, 1.09 mL, 82% purity, 1 eq) in EtOAc (5 mL) was added Pd/C (0.1 g, 10% purity) under Ar. Then the mixture was degassed under vacuum and purged with H₂ three times. The mixture was stirred under H₂ (15 psi) at 25° C. for 1 hour. TLC showed the reaction was completed. The reaction mixture was filtered and the filter was concentrated. The crude product was purified by (column: Waters Xbridge Prep OBD C18 150*40 mm*10 um; mobile phase: [water(10 mM NH₄HCO₃)-ACN]; B %: 30%-60%, 8 min) to get (2R)-2-(4-cyclopentylbutanoyl amino)-N-(2-phenylethyl)butanediamide (37 mg, 99% purity, 100% ee) a white solid.

### Example 229: (R)-8-((4-amino-1,4-dioxo-1-(phenethylamino)butan-2-yl)amino)-8-oxooctanoic acid

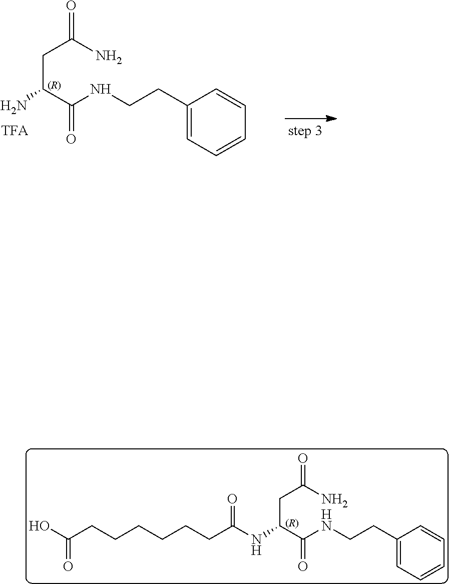

Oxonane-2,9-dione (0.221 g, 1.42 mmol, 1.1 eq) was added to a stirring solution of (1R)-2-carbamoyl-1-[(2-phenylethyl)carbamoyl]ethan-1-aminium trifluoroacetate (0.450 g, 1.29 mmol, 1.0 eq) and N,N-diisopropylethylamine (0.67 mL, 3.87 mmol, 3.0 eq) in anhydrous dichloromethane (5 mL). After 24 h, LC-MS analysis confirmed the formation of the desired product. Water was added to the reaction mixture and the precipitate was filtered off (side product: N-[(1R)-2-carbamoyl-1-[(2-phenylethyl)carbamoyl]ethyl]-N′-{2-carbamoyl-1-[(2-phenylethyl)carbamoyl]ethyl}octanediamide, white solid). The solvents were further evaporated and crude was purified by preparative HPLC to give 7-{1[(1R)-2-carbamoyl-1-[(2-phenylethyl)carbamoyl]ethyl]carbamoyl}heptanoic acid as a white solid (47 mg, 9%).

¹H NMR (400 MHz, DMSO-d₆) δ 12.28 (s, 1H), 7.94 (d, J=8.1 Hz, 1H), 7.80 (t, J=5.7 Hz, 1H), 7.34-7.14 (m, 6H), 6.85 (s, 1H), 4.50 (td, J=7.9, 5.8 Hz, 1H), 3.29-3.20 (m, 2H), 2.68 (t, J=7.4 Hz, 2H), 2.45 (dd, J=15.2, 5.9 Hz, 1H), 2.37-2.27 (m, 1H), 2.18 (t, J=7.4 Hz, 2H), 2.09 (t, J=7.5 Hz, 2H), 1.47 (h, J=7.1 Hz, 4H), 1.34-1.14 (m, 4H).

### Example 230: (R)-2-acetamido-N′-phenethylsuccinamide

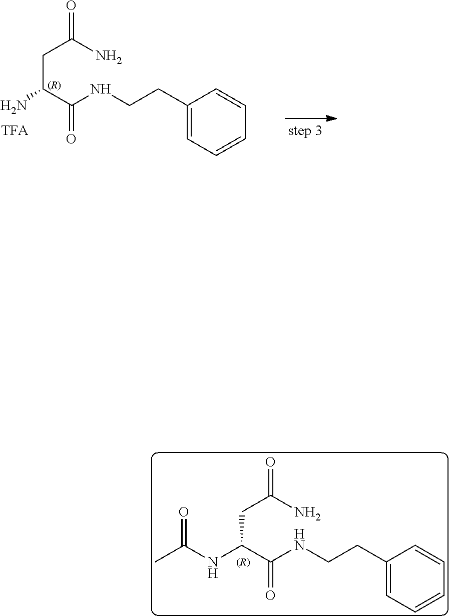

Acetic anhydride (0.104 ml, 1.1 mmol, 1.5 eq) was added to a stirring solution of (1R)-2-carbamoyl-1-[(2-phenylethyl)carbamoyl]ethan-1-aminium trifluoroacetate (0.284 g, 0.732 mmol, 1.0 eq) and N,N-diisopropylethylamine (0.284 g, 2.197 mmol, 3.0 eq) in anhydrous dichloromethane (3 mL). After 1 h, LC-MS analysis confirmed the formation of the desired product. Water was added to the mixture and the precipitate was filtered and washed subsequently with NH₄Cl, 1M NaOH and water.

(2R)-2-acetamido-N-(2-phenylethyl)butanediamide (0.053 g, 26%) was obtained as a white solid.

¹H NMR (300 MHz, DMSO-d₆): δ 7.98 (d, J=8.1 Hz, 1H), 7.82 (t, J=5.8 Hz, 1H), 7.35-7.12 (m, 6H), 6.85 (s, 1H), 4.49 (td, J=7.8, 5.6 Hz, 1H), 3.30-3.16 (m, 2H), 2.68 (t, J=7.4 Hz, 2H), 2.47-2.40 (m, 1H), 2.31 (dd, J=15.2, 7.9 Hz, 1H), 1.83 (s, 3H).

### Example 231: (R)-2-decanamido-N′-phenethylsuccinamide

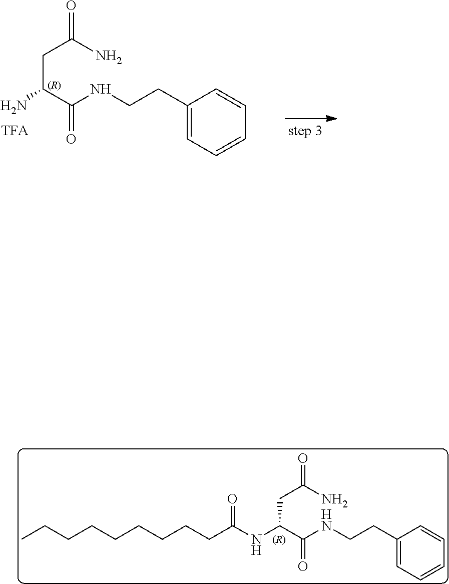

To a stirring solution of (1R)-2-carbamoyl-1-[(2-phenylethyl)carbamoyl]ethan-1-aminium trifluoroacetate (0.284 g, 0.732 mmol, 1.0 eq) and N,N-diisopropylethylamine (0.284 g, 2.20 mmol, 3.0 eq) in anhydrous DMF (2.5 mL) was added decanoic anhydride (0.359 g, 1.10 mmol, 1.5 eq). After 1 h, LC-MS analysis showed the formation of the desired product. Water was added to the reaction mixture and the precipitate was subsequently washed with NH₄Cl, 1M NaOH and water to give (2R)-2-decanamido-N-(2-phenylethyl)butanediamide (0.188 g, 0.483 mmol, 48%) as a white solid.

¹H NMR (300 MHz, DMSO-d₆): δ 7.99 (d, J=8.0 Hz, 1H), 7.82 (t, J=5.7 Hz, 1H), 7.33-7.17 (m, 6H), 6.83 (s, 1H), 4.50 (q, J=7.4 Hz, 1H), 3.25 (q, J=7.2, 6.6 Hz, 2H), 2.68 (t, J=7.4 Hz, 2H), 2.46-2.41 (m, 1H), 2.32 (dd, J=15.3, 7.7 Hz, 1H), 2.08 (t, J=7.5 Hz, 2H), 1.50-1.40 (m, 2H), 1.24 (s, 13H), 0.86 (t, J=6.6 Hz, 3H).

### Example 232: (R)-2-hexanamido-N1-phenethylsuccinamide

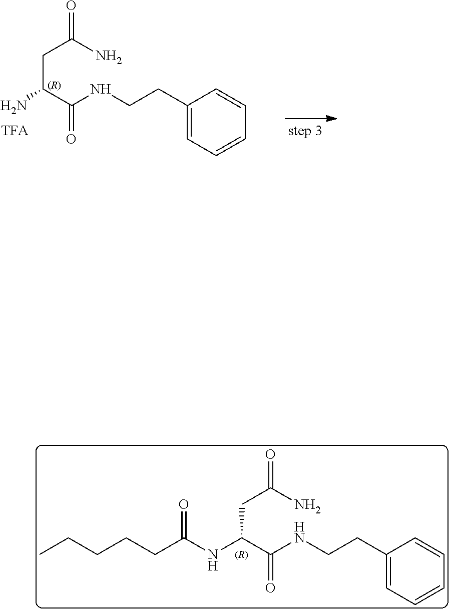

Hexanoic anhydride (0.236 g, 1.10 mmol, 1.5 eq) was added to a solution of (1R)-2-carbamoyl-1-[(2-phenylethyl)carbamoyl]ethan-1-aminium trifluoroacetate (0.284 g, 0.732 mmol, 1.0 eq) and N,N-diisopropylethylamine (0.284 g, 2.20 mmol, 3.0 eq) in anhydrous DMF (2.5 mL). After 1 h, LC-MS analysis confirmed the formation of the desired product. Water was added to the mixture and the precipitate was filtered and washed with 1M NaOH and water to give (2R)-2-hexanamido-N-(2-phenylethyl)butanediamide (0.20 g, 59%) as a white solid.

¹H NMR (300 MHz, DMSO-d₆): δ 7.91 (d, J=8.1 Hz, 1H), 7.78 (t, J=5.7 Hz, 1H), 7.32-7.16 (m, 6H), 6.84 (s, 1H), 4.50 (td, J=7.8, 5.8 Hz, 1H), 3.25 (dt, J=8.5, 6.7 Hz, 2H), 2.68 (t, J=7.4 Hz, 2H), 2.47-2.41 (m, 1H), 2.31 (dd, J=15.2, 7.8 Hz, 1H), 2.08 (t, J=7.4 Hz, 2H), 1.47 (p, J=7.4 Hz, 2H), 1.25 (ddt, J=12.5, 9.7, 5.9 Hz, 4H), 0.86 (t, J=6.8 Hz, 3H).

### Example 233: (R)-2-butyramido-N′-phenethylsuccinamide

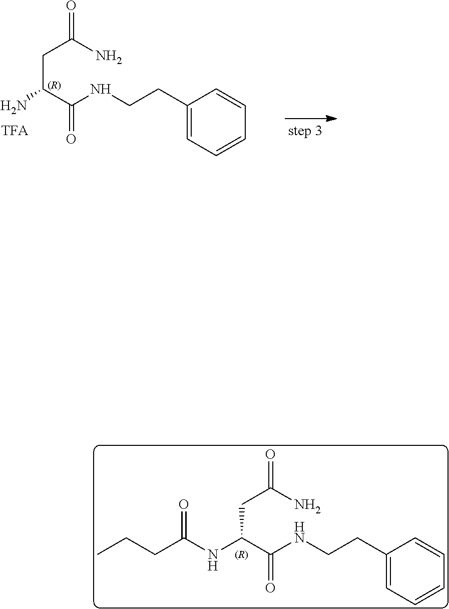

To a solution of (1R)-2-carbamoyl-1-[(2-phenylethyl)carbamoyl]ethan-1-aminium trifluoroacetate (0.278 g, 0.716 mmol, 1.0 eq) and N,N-diisopropylethylamine (0.278 g, 2.15 mmol, 3.0 eq) in anhydrous DMF (2.5 mL) was added butyric anhydride (0.17 g, 1.08 mmol, 1.5 eq). After 1 h, LC-MS analysis confirmed the formation of the desired product. Water was added to the reaction mixture and precipitate was washed subsequently with NH₄Cl, 1M NaOH and water to give (2R)-2-butanamido-N-(2-phenylethyl)butanediamide (0.053 g, 17%) as a white solid.

¹H NMR (300 MHz, DMSO-d₆): δ 7.91 (d, J=8.1 Hz, 1H), 7.79 (t, J=5.7 Hz, 1H), 7.33-7.16 (m, 6H), 6.85 (s, 1H), 4.50 (td, J=7.9, 5.8 Hz, 1H), 3.25 (qd, J=7.3, 2.5 Hz, 2H), 2.68 (t, J=7.4 Hz, 2H), 2.47-2.41 (m, 1H), 2.31 (dd, J=15.2, 7.9 Hz, 1H), 2.07 (t, J=7.3 Hz, 2H), 1.49 (h, J=7.4 Hz, 2H), 0.84 (t, J=7.4 Hz, 3H).

### Example 234: (R)-N′-phenethyl-2-(4-phenylbutanamido)succinamide

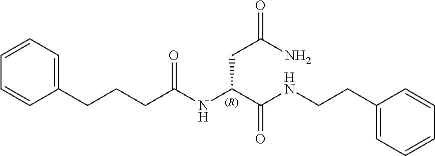

### Step 1: Synthesis of 2-(4-phenylbutanamido)-N-(2-phenylethyl)-N′-(triphenylmethyl)-butanediamide

To a solution of 2-amino-N-(2-phenylethyl)-N′-(triphenylmethyl)butanediamide (0.200 g, 0.42 mmol, 1.0 eq) and 4-phenylbutyric acid (0.07 g, 0.42 mmol, 1.0 eq) in anhydrous ACN (5 ml) was added HBTU (0.19 g, 0.50 mmol, 1.2 eq) and N,N-diisopropylethylamine (0.06 g, 0.46 mmol, 1.1 eq). After 12 h, the LCMS analysis confirmed the formation of desired product. The precipitate was filtered, washed with water and then acetonitrile, and dried under reduced pressure to give 2-(4-phenylbutanamido)-N-(2-phenylethyl)-N′-(triphenylmethyl)butanediamide (0.15 g, 56%) as a white solid.

### Step 2: Synthesis of amino-N-(2-phenylethyl)butanediamide

To a solution of 2-(4-phenylbutanamido)-N-(2-phenylethyl)-N′-(triphenylmethyl)butanediamide (0.15 g, 0.24 mmol, 1.0 eq) in dichloromethane (2 ml) was added water (0.1 mL, 20.0 eq), triethylsilane (0.05 ml, 0.48 mmol, 2.0 eq) and finally trifluoroacetic acid (3.0 ml, 520 mmol, 100.0 eq). After 12 h, LC-MS analysis confirmed the formation of the desired product. Water was added and the aqueous phase was washed with dichloromethane. After that, the aqueous layer was dried under reduced pressure and was further purified by flash column chromatography (DCM:MeOH, 9:1) to give 2-amino-N-(2-phenylethyl)butanediamide (0.046 g, 49%) as a white solid.

¹H NMR (400 MHz, DMSO-d₆) δ 7.94 (d, J=8.1 Hz, 1H), 7.79 (t, J=5.7 Hz, 1H), 7.33-7.22 (m, 5H), 7.22-7.14 (m, 6H), 6.85 (s, 1H), 4.51 (td, J=8.0, 5.7 Hz, 1H), 3.31-3.17 (m, 2H), 2.68 (t, J=7.4 Hz, 2H), 2.60-2.53 (m, 2H), 2.45 (dd, J=15.2, 5.7 Hz, 1H), 2.38-2.27 (m, 1H), 2.12 (t, J=7.5 Hz, 2H), 1.77 (p, J=7.6 Hz, 2H).

### Example 235: (R)-2-(4-cyclohexylbutanamido)-N′-phenethylsuccinamide

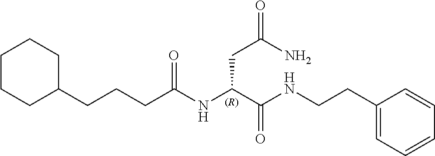

Step 1: Synthesis of 2-(4-cyclohexylbutanamido)-N-(2-phenylethyl)-N′-(triphenylmethyl)butanediamide

To a solution of 2-amino-N-(2-phenylethyl)-N′-(triphenylmethyl)butanediamide (0.200 g, 0.42 mmol, 1.0 eq) and 4-cyclohexanelbutyric acid (0.07 g, 0.42 mmol, 1.0 eq) in anhydrous ACN (5 ml) was added HBTU (0.19 g, 0.50 mmol, 1.2 eq) and N,N-diisopropylethylamine (0.06 g, 0.46 mmol, 1.1 eq). After 12 h, the LCMS analysis confirmed the formation of desired product. The precipitate was filtered, washed with water, acetonitrile and dried under reduced pressure to give 2-(4-cyclohexylbutanamido)-N-(2-phenylethyl)-N′-(triphenylmethyl)butanediamide (0.14 g, 52%) as a white solid.

### Step 2: Synthesis of 2-(4-cyclohexylbutanamido)-N-(2-phenylethyl)butanediamide

To a solution 2-(4-cyclohexylbutanamido)-N-(2-phenylethyl)-N′-(triphenylmethyl)butanediamide (0.14 g, 0.24 mmol, 1.0 eq) in dichloromethane (2 ml) was added water (0.1 mL, 20.0 eq), triethylsilane (0.05 ml, 0.48 mmol, 2.0 eq) and finally trifluoroacetic acid (3.0 ml, 520 mmol, 100.0 eq). After 12 h, LC-MS analysis confirmed the formation of the desired product. Then water was added to the reaction mixture and the aqueous phase was washed with dichloromethane. After that, the aqueous layer was dried under reduced pressure and further purified by flash column chromatography (DCM:MeOH; 9:1) to give 2-(4-cyclohexylbutanamido)-N-(2-phenylethyl)butanediamide (0.035 g, 35%) as a white solid.

¹H NMR (400 MHz, DMSO-d₆) δ 7.90 (d, J=8.1 Hz, 1H), 7.77 (t, J=5.7 Hz, 1H), 7.32-7.15 (m, 6H), 6.84 (s, 1H), 4.50 (td, J=7.9, 5.9 Hz, 1H), 3.29-3.20 (m, 2H), 2.68 (t, J=7.4 Hz, 2H), 2.45 (dd, J=15.2, 6.0 Hz, 1H), 2.36-2.26 (m, 1H), 2.06 (t, J=7.5 Hz, 2H), 1.72-1.56 (m, 5H), 1.48 (p, J=7.6 Hz, 2H), 1.27-1.04 (m, 6H), 0.91-0.75 (m, 2H).

### Example 236: (R)-2-(2-cyclohexylacetamido)-N′-phenethylsuccinamide

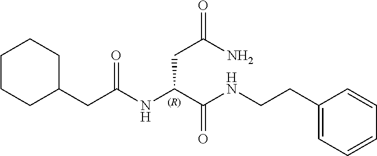

### Step 1: Synthesis of 2-(2-cyclohexylacetamido)-N-(2-phenylethyl)-N′-(triphenylmethyl)butanediamide

To a solution of 2-amino-N-(2-phenylethyl)-N′-(triphenylmethyl)butanediamide (0.200 g, 0.42 mmol, 1.0 eq) and cyclohexaneacetic acid (0.06 g, 0.42 mmol, 1.0 eq) in anhydrous ACN (5 ml) was added HBTU (0.19 g, 0.50 mmol, 1.2 eq) and N,N-diisopropylethylamine (0.06 g, 0.46 mmol, 1.1 eq). After 12 h, the LCMS analysis confirmed the formation of desired product. The precipitate was filtered, washed with water, acetonitrile and dried under reduced pressure to give 2-(2-cyclohexylacetamido)-N-(2-phenylethyl)-N′-(triphenylmethyl)butanediamide (0.135 g, 53%) as a white solid.

### Step 2: Synthesis of 2-(2-cyclohexylacetamido)-N-(2-phenylethyl)butanediamide

To a solution 2-(2-cyclohexylacetamido)-N-(2-phenylethyl)-N′-(triphenylmethyl)butanediamide (0.15 g, 0.24 mmol, 1.0 eq) in dichloromethane (2 ml) was added water (0.1 mL, 20.0 eq), triethylsilane (0.05 ml, 0.48 mmol, 2.0 eq) and finally trifluoroacetic acid (3.0 ml, 520 mmol, 100 eq). After 12 h, LC-MS analysis confirmed the formation of the desired product and the consumption of the starting material. Water were added to the reaction mixture and the aqueous phase was washed with dichloromethane. After that, the aqueous layer was dried under reduced pressure and further purified by flash column chromatography (DCM:MeOH, 9:1) to give 2-(2-cyclohexylacetamido)-N-(2-phenylethyl)butanediamide (0.018 g, 32%) as a white solid.

¹H NMR (400 MHz, DMSO-d₆) δ 7.91 (d, J=8.1 Hz, 1H), 7.77 (t, J=5.5 Hz, 1H), 7.35-7.13 (m, 6H), 6.85 (s, 1H), 4.58-4.43 (m, 1H), 3.28-3.17 (m, 2H), 2.69 (t, J=7.7 Hz, 2H), 2.44-2.40 (m, 1H), 2.36-2.26 (m, 1H), 1.97 (d, J=6.7 Hz, 2H), 1.72-1.53 (m, 6H), 1.28-1.03 (m, 3H), 0.97-0.78 (m, 2H).

### Example 237: (R)-N-(1,4-dioxo-1-phenyl-4-(tritylamino)butan-2-yl)octanamide

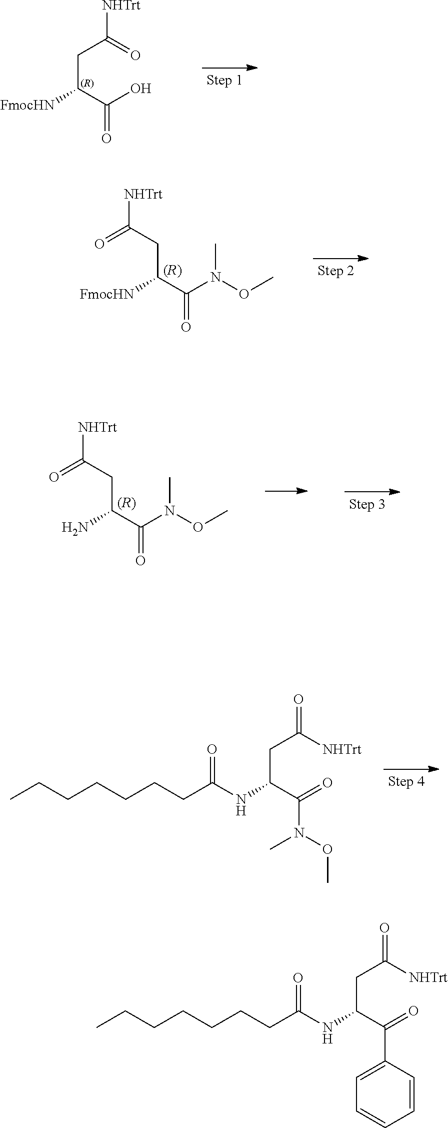

### Step 1: Synthesis of (9H-fluoren-9-yl)methyl N-[(1R)-1-[methoxy(methyl)carbamoyl]-2-[(triphenylmethyl)carbamoyl]ethyl]carbamate

To a solution of (2R)-2-({[(9H-fluoren-9-yl)methoxy]carbonyl}amino)-3-[(triphenylmethyl)carbamoyl]propanoic acid (3.00 g, 5.028 mmol, 1.00 eq) and 1-hydroxybenzotriazole monohydrate (770 mg, 5.028 mmol, 1.00 eq) in anhydrous DMF (25 mL), diisopropylcarbodiimide (DIC, 779 μL, 5.028 mmol, 1.00 eq) was added dropwise. The mixture was stirred at room temperature for 30 min and then followed by the addition of N,N-diisopropylethylamine (DIPEA, 876 μL, 5.028 mmol, 1.00 eq) and N,O-dimethylhydroxylamine hydrochloride (490 mg, 5.028 mmol, 1.00 eq) portionwise. The reaction was continued for 72 h and monitored by TLC (75% AcOEt in Hexane) and LC-MS. The mixture was diluted with AcOEt, washed with water, sat. Na₂CO₃ and brine. Organic layer was dried over anhydrous Na₂SO₄, filtered and concentrated. The residue was purified by FCC (Hexane—75% AcOEt in Hexane). Product as a white solid (2.94 g, 91%).

¹H NMR (300 MHz, DMSO-d₆) δ 8.52 (s, 1H), 7.89 (d, J=7.5 Hz, 2H), 7.73 (dd, J=7.6, 3.9 Hz, 2H), 7.47-7.35 (m, 2H), 7.35-7.25 (m, 2H), 7.25-7.12 (m, 15H), 5.49 (d, J=7.7 Hz, 1H), 4.79-4.61 (m, 1H), 4.38 (dd, J=9.9, 6.5 Hz, 1H), 4.32-4.15 (m, 2H), 3.64 (s, 3H), 3.10 (s, 3H), 2.61 (dd, J=14.3, 9.7 Hz, 1H), 2.49-2.37 (m, 1H).

### Step 2: Synthesis of (2R)-2-amino-N-methoxy-N-methyl-N′-(triphenylmethyl)-butanediamide

To a solution of (9H-fluoren-9-yl)methyl N-[(1R)-1-[methoxy(methyl)carbamoyl]-2-[(triphenylmethyl)carbamoyl]ethyl]carbamate (2.930 g, 4.580 mmol, 1.00 eq) in MeCN (25 mL), piperidine (2.714 mL, 27.479 mmol, 6 eq) was added dropwise. The reaction was stirred vigorously for 8 h at room temperature during which time a thick white solid appeared. Precipitate was filtered off, washed with hot hexanes and dried to afford the title compound (1.72 g, 90%) as a white solid.

¹H NMR (300 MHz, DMSO-d₆) δ 9.17 (s, 1H), 7.33-7.10 (m, 15H), 3.98-3.88 (m, 1H), 3.63 (s, 3H), 3.10 (s, 3H), 2.45-2.25 (m, 2H).

### Step 3: Synthesis of (2R)-N-methoxy-N-methyl-2-octanamido-N′-(triphenylmethyl)-butanediamide

(2R)-2-amino-N-methoxy-N-methyl-N′-(triphenylmethyl)-butanediamide (1.717 g, 4.112 mmol, 1.00 eq) was dissolved in anhydrous DMF (12 mL). N,N-diisopropylethylamine (DIPEA, 1.576 mL, 9.047 mmol, 2.00 eq) was added, followed by dropwise addition of n-caprylic anhydride (1.344 mL, 4.524 mmol, 1.1 eq). The reaction was carried out at room temperature for 2 h until substrate was fully consumed. Reaction mixture was then diluted in AcOEt and washed with water and brine. Organic layer was dried over Na₂SO₄, filtered and concentrated. The residue was purified by FCC (Hexane—AcOEt 100%) to afford the title compound as a white solid (1.810 g, 77%).

¹H NMR (300 MHz, DMSO-d₆) δ 8.52 (s, 1H), 8.15 (d, J=8.1 Hz, 1H), 7.30-7.11 (m, 15H), 5.02-4.87 (m, 1H), 3.65 (s, 3H), 3.09 (s, 3H), 2.54 (d, J=6.7 Hz, 2H), 2.10 (t, J=7.4 Hz, 2H), 1.47 (t, J=5.9 Hz, 2H), 1.24 (s, 8H), 0.89-0.80 (m, 3H).

### Step 4: Synthesis of N-[(2R)-1-oxo-1-phenyl-3-[(triphenylmethyl)carbamoyl]propan-2-yl]octanamide

(2R)-N-methoxy-N-methyl-2-octanamido-N′-(triphenylmethyl)-butanediamide (300 mg, 0.552 mmol, 1.00 eq) was dissolved in anhydrous THF (4 mL) and cooled to −78° C. under inert atmosphere. Phenyllithium (1.9 M in dibutyl ether, 0.929 mL, 1.766 mmol, 3.20 eq) was slowly added dropwise. The reaction was continued at −78° C. for 80 min and monitored by TLC (50% AcOEt in Hexane)/LC-MS. Upon completion of the reaction, the temperature was increased up to 0° C. and sat. NH₄Cl was added slowly. The mixture was diluted with AcOEt and extracted. Organic layer was dried over anhydrous Na₂SO₄, filtered and concentrated. The residue was purified by FCC (Hexane—50% AcOEt in Hexane) to afford the title compound as a white solid (182 mg, 59%)

¹H NMR: ¹H NMR (300 MHz, Chloroform-d) δ 7.82 (d, J=7.7 Hz, 2H), 7.53 (t, J=7.3 Hz, 1H), 7.40 (t, J=7.6 Hz, 2H), 7.33-7.27 (m, 3H), 7.24-7.11 (m, 7H), 7.05 (d, J=8.5 Hz, 1H), 6.87 (s, 1H), 5.60 (dt, J=8.7, 4.5 Hz, 1H), 3.01-2.73 (m, 2H), 2.17-1.97 (m, 2H), 1.55-1.40 (m, 2H), 1.30-1.22 (m, 2H), 1.22-1.10 (m, 6H), 0.85 (t, J=6.7 Hz, 3H).

### Example 238: Ethyl (R,E)-6-amino-4-octanamido-6-oxohex-2-enoate

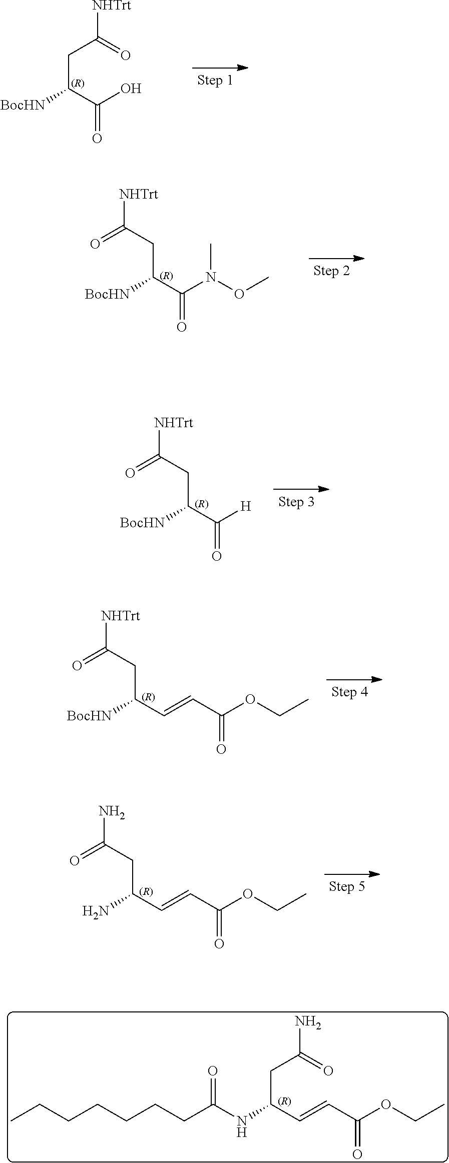

Step 1: Synthesis of tert-butyl N-[(2R)-1-oxo-3-[(triphenylmethyl)carbamoyl]propan-2-yl]carbamate

To a solution of N-[(1R)-1-[methoxy(methyl)carbamoyl]-2-[(triphenylmethyl)carbamoyl]-ethyl]carbamate (1.00 g, 1.93 mmol, 1.0 eq), in THF (25 mL) LiAlH₄ (2 mL, 2M sol. in THF, 2.0 eq) was added and the mixture was stirred at −15° C. for 1 h. The reaction was controlled by LC-MS. After full consumption of starting material reaction mixture was quenched with water and diluted with DCM. The organic layer was washed with water, dried over Na₂SO₄, filtered and concentrated under reduced pressure to give tert-butyl N-[(2R)-1-oxo-3-[(triphenylmethyl)carbamoyl]propan-2-yl]carbamate (0.10 g, crude). The crude product was used in the next step without purification.

Note: The formation of intramolecular condensation product was observed (main product).

### Step 2: Synthesis of ethyl (2E,4R)-4-{[(tert-butoxy)carbonyl]amino}-5-[(triphenylmethyl)carbamoyl]pent-2-enoate

To a solution of crude tert-butyl N-[(2R)-1-oxo-3-[(triphenylmethyl)carbamoyl]propan-2-yl]carbamate (1.00 g, 2.18 mmol, 1.0 eq), in THF (25 mL) ethyl 2-(diethoxyphosphoryl)acetate (0.98 g, 4.37 mmol 2.0 eq) and potassium tert-butoxide (0.49 g, 4.37 mmol, 2.0 eq) were added and mixture was stirred at 20° C. for 1 h. The reaction was monitored by LC-MS. After 2 hours solvent was removed and the residue was diluted with ethyl acetate. The organic layer was washed with water, NH₄Cl solution, dried over Na₂SO₄, filtered and concentrated under reduced pressure. The crude was purified by flash column chromatograph (20% EtOAc in hexane) to give ethyl (2E,4R)-4-{[(tert-butoxy)carbonyl]amino}-5-[(triphenylmethyl)carbamoyl]pent-2-enoate (0.11 g, 8%).

¹H NMR (300 MHz, DMSO-d₆) δ 7.34-7.10 (m, 15H), 6.77 (dd, J=15.6, 5.2 Hz, 1H), 5.83-5.72 (m, 1H), 4.14 (d, J=7.1 Hz, 2H), 4.04 (s, 1H), 1.39 (s, 9H), 1.22 (t, J=7.1 Hz, 3H).

### Step 3: Synthesis of ethyl (2E,4R)-4-amino-5-carbamoylpent-2-enoate

To a solution of ethyl (2E,4R)-4-{[(tert-butoxy)carbonyl]amino}-5-[(triphenylmethyl)-carbamoyl]pent-2-enoate (0.095 g, 0.17 mmol, 1.0 eq) in dichloromethane (2 ml) was added water (0.2 mL, 10.0 eq) and trifluoroacetic acid (0.40 ml, 3.41 mmol, 20.0 eq). After 12 h, LC-MS analysis confirmed consumption of the starting material. Water was added to the reaction mixture and the aqueous phase was washed with dichloromethane. After that, the aqueous layer was concentrated under reduced pressure to give ethyl (2E,4R)-4-amino-5-carbamoylpent-2-enoate (0.030 g, 93%) as a yellow solid. Crude product was used in the next step without purification.

### Step 4: Synthesis of ethyl (2E,4R)-5-carbamoyl-4-octanamidopent-2-enoate

To a stirring solution of crude ethyl (2E,4R)-4-amino-5-carbamoylpent-2-enoate (0.028 g, 0.16 mmol, 1.0 eq) and N,N-diisopropylethylamine (0.04 g, 0.32 mmol, 2.0 eq) in anhydrous ACN (2 mL) was added octanoic anhydride (0.090 g, 0.32 mmol, 2.0 eq). After 1 h, LC-MS analysis showed the formation of desired product. The solvent was evaporated and crude product was purified by preparative HPLC to give ethyl (2E,4R)-5-carbamoyl-4-octanamidopent-2-enoate (0.007 g, 13%) as a white solid.

¹H NMR (400 MHz, DMSO-d₆) δ 8.01 (d, J=8.2 Hz, 1H), 7.38 (s, 1H), 6.98-6.72 (m, 2H), 5.81 (dd, J=15.7, 1.8 Hz, 1H), 4.78 (t, J=6.7 Hz, 1H), 4.12 (q, J=7.1 Hz, 2H), 2.42-2.25 (m, 2H), 2.10 (td, J=7.3, 3.5 Hz, 2H), 1.50 (d, J=9.4 Hz, 2H), 1.34-1.07 (m, 11H), 0.86 (t, J=6.7 Hz, 3H).

### Example 239: (R)-N′-(4-hydroxyphenethyl)-N1-isopropyl-2-octanamidosuccinamide

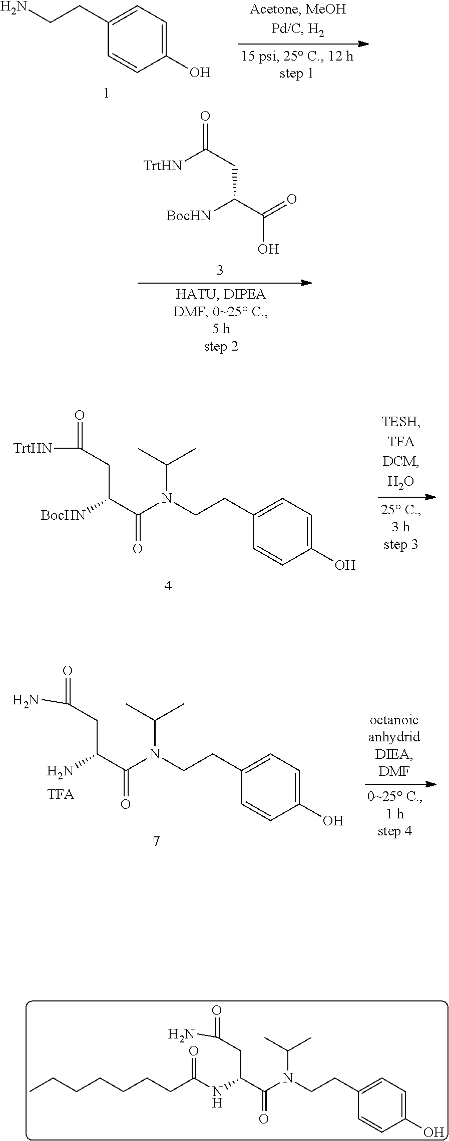

### Step 1

To a solution of 4-(2-aminoethyl)phenol (200 mg, 1.46 mmol, 1 eq) and acetone (84.68 mg, 1.46 mmol, 107.19 uL, 1 eq) in MeOH (5 mL) was added Pd/C (10%, 0.08 g) under H₂ atmosphere. The suspension was degassed and purged with H₂ for 3 times. The mixture was stirred under H₂ (15 Psi) at 25° C. for 12 hr. The reaction mixture was filtered and filtrate concentrated under reduced pressure to give a residue. The crude product was triturated with PE:EA=10:1 (22 mL) at 25° C. for 20 min. Then filtered and filter cake concentrated under reduced pressure to give compound 4-[2-(isopropylamino)ethyl]phenol (230 mg, 1.28 mmol, 88.00% yield) as a white solid.

### Step 2

To a solution of (2R)-2-(tert-butoxycarbonylamino)-4-oxo-4-(tritylamino)butanoic acid (0.5 g, 1.05 mmol, 1 eq) and 4-[2-(isopropylamino)ethyl]phenol (226.65 mg, 1.26 mmol, 1.2 eq) in DMF (30 mL) added HATU (480.75 mg, 1.26 mmol, 1.2 eq) and DIPEA (272.34 mg, 2.11 mmol, 367.04 uL, 2 eq) at 0° C. The mixture was warmed to 25° C. and stirred for 3 hr. The reaction mixture was poured into H₂O (50 mL) slowly, filtered and filter cake concentrated under reduced pressure to give a residue. The crude product was triturated with PE:EA=10:1 (44 mL) at 25° C. for 30 min. filtered and filter cake concentrated under reduced pressure to give compound tert-butyl N-[(1R)-1-[2-(4-hydroxyphenyl)ethyl-isopropyl-carbamoyl]-3-oxo-3-(tritylamino) propyl]carbamate (520 mg, 817.88 umol, 77.62% yield) as a white solid.

### Step 3

A solution of tert-butyl N-[(1R)-1-[2-(4-hydroxyphenyl)ethyl-isopropyl-carbamoyl]-3-oxo-3-(tritylamino)propyl]carbamate (520 mg, 817.88 umol, 1 eq, TES (0.25 mL), DCM (0.5 mL), TFA (9 mL) and H₂O (0.25 mL) was stirred at 25° C. for 3 hr. The reaction mixture was concentrated under reduced pressure to give product (2R)-2-amino-N-[2-(4-hydroxyphenyl)ethyl]-N-isopropyl-butanediamide (200 mg, 490.94 umol, 60.03% yield, TFA) as a white solid.

### Step 4

To a solution of (2R)-2-amino-N-[2-(4-hydroxyphenyl)ethyl]-N-isopropyl-butanediamide (200 mg, 490.94 umol, 1 eq, TFA) and octanoyl octanoate (159.30 mg, 589.13 umol, 1.2 eq) in DMF (1 mL) was added DIEA (190.35 mg, 1.47 mmol, 256.54 uL, 3 eq) at 0° C. The mixture was warmed to 25° C. and stirred for 3 hr. The reaction mixture was pour into H₂O (50 mL) slowly, filtered and filter cake concentrated under reduced pressure to give a residue. The residue was purified by prep-TLC (SiO2, DCM: MeOH=5:1) to give compound (2R)-N-[2-(4-hydroxyphenyl)ethyl]-N-isopropyl-2-(octanoylamino)butanediamide(14.5 mg, 34.18 umol, 6.96% yield, 98.9% purity) as a white solid.

### Example 240: (R)-N′-(4-hydroxyphenethyl)-N1-methyl-2-octanamidosuccinamide

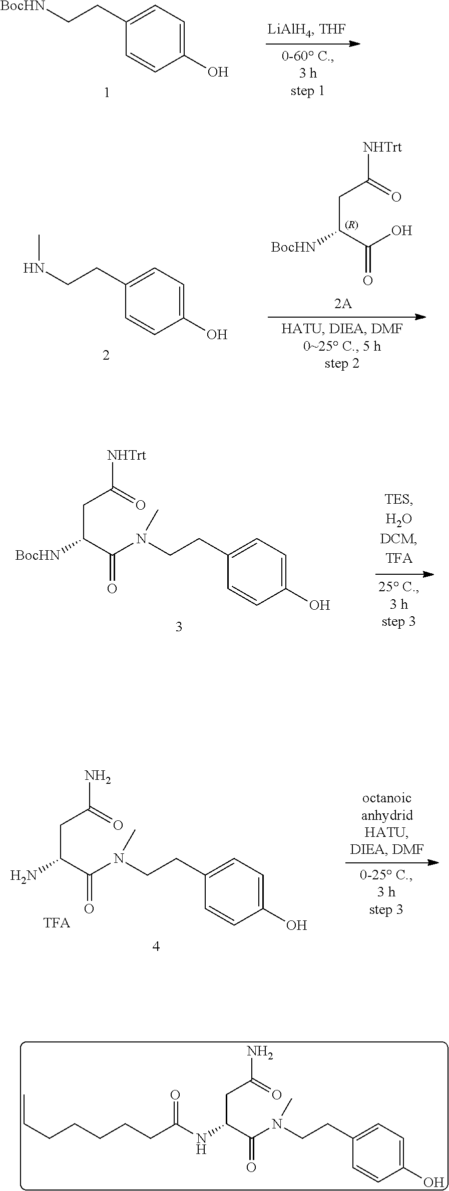

### Step 1

To a solution of LiAlH4 (1.28 g, 33.71 mmol, 4 eq) in THF (20 mL) cooled at 0° C. was added tert-butyl N-[2-(4-hydroxyphenyl)ethyl]carbamate (2 g, 8.43 mmol, 1 eq) slowly. The mixture was warmed to 60° C. and stirred for 3 hr under N₂. The reaction mixture was pour into wet Na₂SO₄ slowly, filtered and concentrated under reduced pressure to give a residue. The crude product was triturated with PE:EA=10:1 (22 mL) at 25° C. for 30 min. filtered and filtrate was concentrated under reduced pressure to give compound 4-[2-(methylamino)ethyl]phenol (480 mg, 3.17 mmol, 37.66% yield) as a white solid.

### Step 2

To a solution of (2R)-2-(tert-butoxycarbonylamino)-4-oxo-4-(tritylamino)butanoic acid (500 mg, 1.05 mmol, 1 eq) and 4-[2-(methylamino)ethyl]phenol (175.25 mg, 1.16 mmol, 1.1 eq) in DMF (10 mL) was added HATU (480.75 mg, 1.26 mmol, 1.2 eq) and DIEA (299.59 mg, 2.32 mmol, 403.75 uL, 2.2 eq) at 0° C. The mixture was warmed to 25° C. and stirred for 5 hr. The reaction mixture was pour into H₂O (30 mL) slowly, filtered and filter cake concentrated under reduced pressure to give a residue. The crude product was triturated with PE:EA=10:1 (22 mL) at 25° C. for 30 min. filtered and filter cake concentrated under reduced pressure to give compound tert-butylN-[(1R)-1-[2-(4-hydroxyphenyl)ethyl-methyl-carbamoyl]-3-oxo-3-(tritylamino)propyl]carbamate (600 mg, 987.27 umol, 93.70% yield) as a white solid.

### Step 3

A solution of tert-butyl N-[(1R)-1-[2-(4-hydroxyphenyl)ethyl-methyl-carbamoyl]-3-oxo-3-(tritylamino)propyl]carbamate (600 mg, 987.27 umol, 1 eq), TFA (18 mL), DCM (1 mE), TES (0.5 mL) and H₂O (0.5 mL) was stirred at 25° C. for 3 hr. The reaction mixture was concentrated under reduced pressure. The crude product (2R)-2-amino-N-[2-(4-hydroxyphenyl)ethyl]-N-methyl-butanediamide (200 mg, 527.24 umol, 53.40% yield, TFA) as a white solid.

### Step 4

The mixture of (2R)-2-amino-N-[2-(4-hydroxyphenyl)ethyl]-N-methyl-butanediamide (200 mg, 527.24 umol, 1 eq, TFA) and octanoyl octanoate (171.08 mg, 632.69 umol, 1.2 eq) in DMF (5 mL) was cooled to 0° C. DIEA (204.43 mg, 1.58 mmol, 275.51 uL, 3 eq) and HATU (240.57 mg, 632.69 umol, 1.2 eq) was added to the mixture. The mixture was warmed to 25° C. and stirred for 3 hr. The reaction mixture was pour into H₂O (50 mL) slowly, filtered and filter cake concentrated under reduced pressure to give a residue. The residue was purified by prep-HPLC (column: Phenomenex luna C18 80*40 mm*3 um; mobile phase: [water(0.1% TFA)-ACN]; B %: 25%-45%, 7 min) to give compound (2R)-N-[2-(4-hydroxyphenyl)ethyl]-N-methyl-2-(octanoylamino)butanediamide (190 mg, 479.00 umol, 90.85% yield, 98.7% purity) as a white solid.

### Example 241: (R)-N′-(2-(4-methylpiperazin-1-yl)ethyl)-2-octanamidosuccinamide

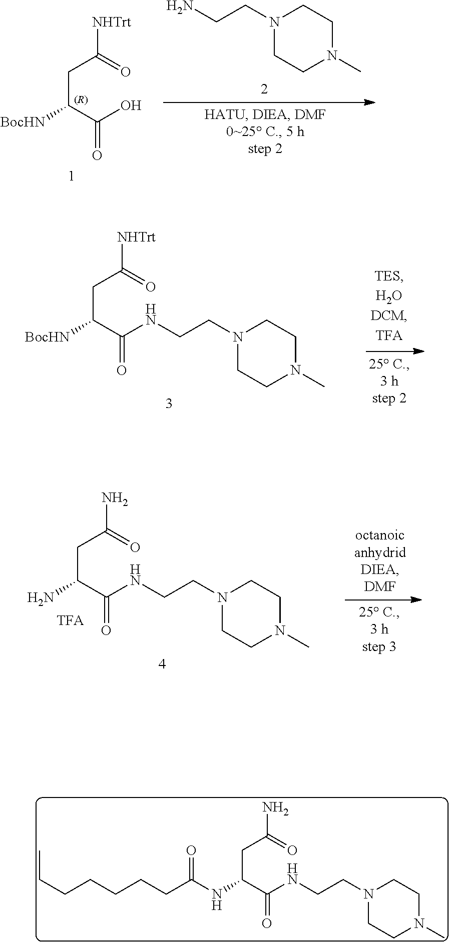

### Step 1

To a mixture of (2R)-2-(tert-butoxycarbonylamino)-4-oxo-4-(tritylamino)butanoic acid (1 g, 2.11 mmol, 1 eq) and 2-(4-methylpiperazin-1-yl)ethanamine (362.19 mg, 2.53 mmol, 1.2 eq) in DMF (30 mL) was added HATU (961.50 mg, 2.53 mmol, 1.2 eq) and DIPEA (599.16 mg, 4.64 mmol, 807.49 uL, 2.2 eq) at 0° C. under N₂. The mixture was stirred at 25° C. for 3 hours. LCMS showed the reaction was completed and desired mass was detected. To the mixture was added water (60 mL) and brine (60 mL). Then the mixture was extracted with ethyl acetate (60 mL*3). The combined organic phase was washed with brine (100 mL), dried with anhydrous Na₂SO₄, filtered and concentrated in vacuum to get tert-butyl N-[(1R)-1-[2-(4-methylpiperazin-1-yl) ethylcarbamoyl]-3-oxo-3-(tritylamino)propyl]carbamate (1 g, crude) as a white solid.

### Step 2

A mixture of tert-butyl N-[(1R)-1-[2-(4-methylpiperazin-1-yl)ethylcarbamoyl]-3-oxo-3-(trityl amino)propyl]carbamate (1 g, 1.67 mmol, 1 eq), TFA (27.72 g, 243.12 mmol, 18 mL, 145.81 eq), DCM (2.64 g, 31.08 mmol, 2 mL, 18.64 eq), H₂O (1.00 g, 55.49 mmol, 1 mL, 33.28 eq), triethylsilane (728.00 mg, 6.26 mmol, 1 mL, 3.76 eq) was stirred at 25° C. for 3 h. LCMS showed the reaction was completed and a little of desired mass was detected. The mixture was concentrated in vacuum to get (2R)-2-amino-N-[2-(4-methylpiperazin-1-yl)ethyl]butanediamide (500 mg, crude) as a white solid.

### Step 3

To a mixture of (2R)-2-amino-N-[2-(4-methylpiperazin-1-yl)ethyl]butanediamide (500 mg, 538.57 umol, 40% purity, 1 eq, TFA) and octanoyl octanoate (218.45 mg, 807.85 umol, 1.5 eq) in DMF (15 mL) was added DIPEA (348.02 mg, 2.69 mmol, 469.03 uL, 5 eq) under N₂. The mixture was stirred at 25° C. for 3 hour. LCMS showed the reaction was completed and desired mass was detected. The mixture was concentrated in vacuum to get a residue. The residue was purified by prep-HPLC (column: Phenomenex Luna C18 100*30 mm*5 um; mobile phase: [water(0.04%/oHCl)-ACN]; B %: 10%-40%, 10 min) to give (2R)-N-[2-(4-methylpiperazin-1-yl)ethyl]-2-(octanoylamino) butanediamide (96 mg, 219.43 umol, 40.74% yield, 96% purity, HCl) as a white solid.

### Example 242: methyl ((S)-4-amino-2-octanamido-4-oxobutyl)-L-alaninate

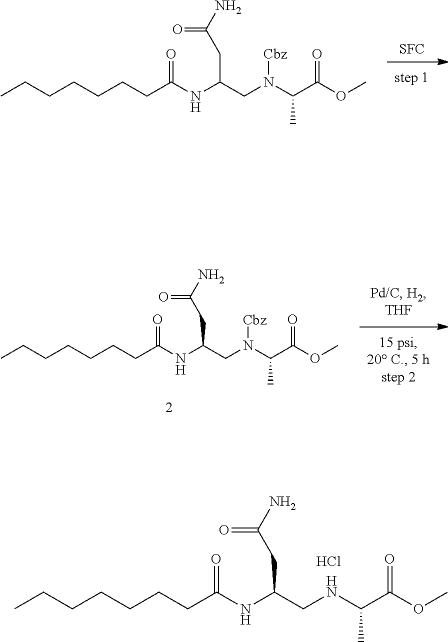

### Step 1

Methyl (2S)-2-[[4-amino-2-(octanoylamino)-4-oxo-butyl]-benzyloxycarbonyl-amino]propanoate (280.00 mg, 604.01 umol, 1 eq) was separated by SFC (column: DAICEL CHIRALPAK AS(250 mm*30 mm, 10 um); mobile phase: [0.1% NH3H2O MEOH]; B %: 30%-20%, 10 min). Compound methyl (2S)-2-[[(2S)-4-amino-2-(octanoylamino)-4-oxo-butyl]-benzyloxycarbonyl-amino]propanoate (40 mg, 79.38 umol, 13.14% yield, 92% purity) was obtained as colorless oil.

### Step 2

To a solution of methyl methyl (2S)-2-[[(2S)-4-amino-2-(octanoylamino)-4-oxo-butyl]-benzyloxycarbonyl-amino]propanoate (30.00 mg, 64.72 umol, 1 eq) in THF (5 mL) was added Pd/C (10 mg, 64.72 umol, 10% purity) under N₂. The suspension was degassed under vacuum and purged with H₂ several times. The mixture was stirred under H₂ (15 psi) at 20° C. for 5 hr. TLC indicated methyl (2S)-2-[[(2S)-4-amino-2-(octanoylamino)-4-oxo-butyl]-benzyloxycarbonyl-amino]propanoate was consumed completely and two new spots formed. The reaction mixture was filtered and the filter was concentrated under reduced pressure to give product. The product was freeze-dried after work-up with HCl (0.25 M, 242.84 uL, 2 eq). Compound methyl (2S)-2-[[(2S)-4-amino-2-(octanoylamino)-4-oxo-butyl]amino]propanoate (25 mg, 65.59 umol, 108.04% yield, 96% purity, HCl) was obtained as a white solid.

### Example 243: (R)-N′-benzyl-3-octanamido-2-oxopentanediamide

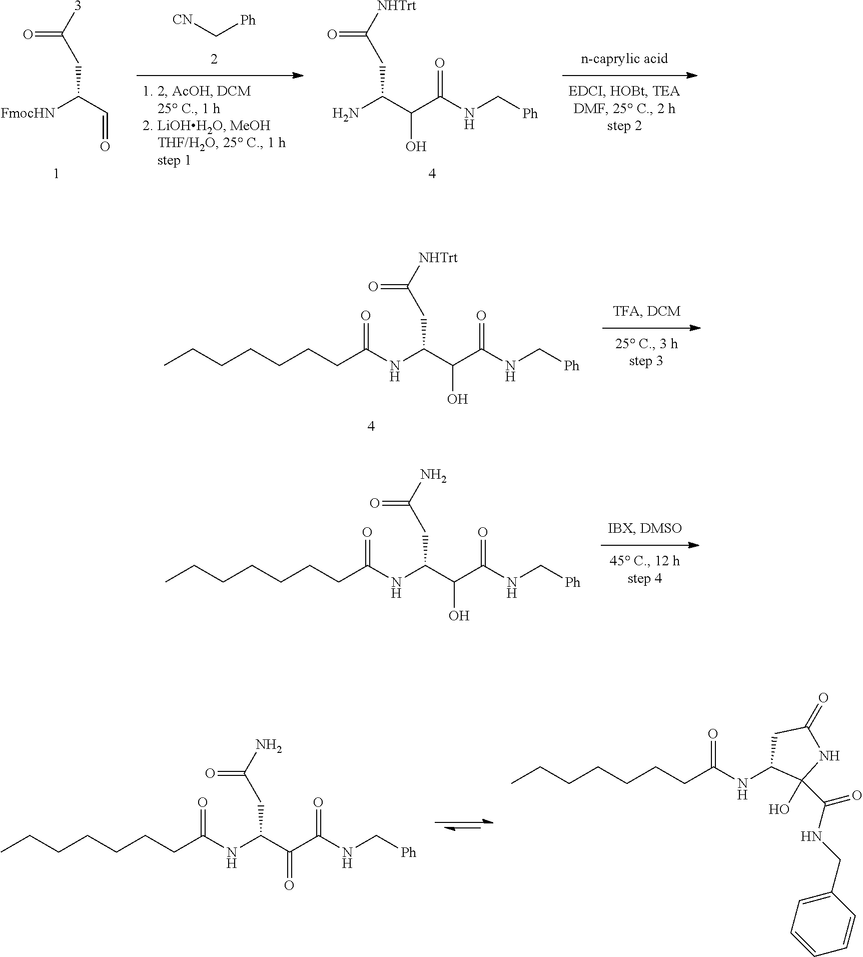

### Step 1

To a solution of 9H-fluoren-9-ylmethyl N-[(1R)-1-formyl-3-oxo-3-(tritylamino)propyl]carbamate (2 g, 3.44 mmol, 1 eq) in DCM (5 mL) was added CH₃COOH (620.51 mg, 10.33 mmol, 590.96 uL, 3 eq) and isocyanomethylbenzene (484.19 mg, 4.13 mmol, 503.32 uL, 1.2 eq) at 25° C. over 1 hr. The solution was concentrated and the obtained residue dissolved in EtOAc (5 mL). The organic phase was washed with 1N HCl (5 mL), 5% NaHCO₃ (10 mL) and brine, dried over Na₂SO₄ and concentrated under reduced pressure to give a residue. The residue in MeOH (1.5 mL), THF (4 mL) and H₂O (0.5 mL) was added LiOH·H₂O (289.07 mg, 6.89 mmol, 2 eq) at 25° C. The mixture was stirred at 25° C. for 1 h. The reaction mixture was concentrated under reduced pressure to give a residue. The residue was purified by prep-HPLC (column: Welch Xtimate C18 250*50 mm*10 um; mobile phase: [water(0.05% NH₃H2O+10 mM NH₄HCO₃)-ACN]; B %: 40%-70%, 30 min) to give compound (3R)-3-amino-N-benzyl-2-hydroxy-N′-trityl-pentanediamide (500 mg, 1.01 mmol, 29.41% yield) as a white solid.

### Step 2

To a solution of (3R)-3-amino-N-benzyl-2-hydroxy-N′-trityl-pentanediamide (300 mg, 607.79 umol, 1 eq) and octanoic acid (131.47 mg, 911.68 umol, 144.48 uL, 1.5 eq) in DMF (5 mL) was added EDCI (128.17 mg, 668.56 umol, 1.1 eq), HOBt (90.34 mg, 668.56 umol, 1.1 eq) and TEA (123.00 mg, 1.22 mmol, 169.19 uL, 2 eq). The mixture was stirred at 25° C. for 2 hr. The reaction mixture was diluted with H₂O 100 mL and filtered and filter cake concentrated under reduced pressure to give a residue. The crude product was triturated with H₂O (5 mL) and PE:EA=10:1 (5.5 mL) and filtered. The filter cake concentrated under reduced pressure to give compound (3R)-N-benzyl-2-hydroxy-3-(octanoylamino)-N′-trityl-pentanediamide (195 mg, 314.62 umol, 51.77% yield) as a white solid.

### Step 3

To a solution of (3R)-N-benzyl-2-hydroxy-3-(octanoylamino)-N′-trityl-pentanediamide (200 mg, 322.69 umol, 1 eq) in DCM (5 mL) was added TFA (1.54 g, 13.51 mmol, 1 mL, 41.85 eq). The mixture was stirred at 25° C. for 3 hr. The reaction mixture was concentrated under reduced pressure to remove DCM (5 mL) and TFA (1 mL). The residue was purified by prep-HPLC (column: Phenomenex Gemini-NX C18 75*30 mm*3 um; mobile phase: [water(10 mM NH4HCO3)-ACN]; B %: 20%-50%, 12 min) to give compound (3R)-N-benzyl-2-hydroxy-3-(octanoylamino)pentanediamide (27 mg, 71.53 umol, 22.17% yield) as a white solid.

### Step 4

To a solution of (3R)-N-benzyl-2-hydroxy-3-(octanoylamino)pentanediamide (25 mg, 66.23 umol, 1 eq) in DMSO (1 mL) was added IBX (74.18 mg, 264.92 umol, 4 eq). The mixture was stirred at 45° C. for 12 hr. LC-MS showed desired MS was detected. The reaction mixture was concentrated under reduced pressure to give a residue. The residue was purified by prep-HPLC (column: Phenomenex Gemini-NX C18 75*30 mm*3 um; mobile phase: [water(10 mM NH₄HCO₃)-ACN]; B %: 20%-45%, 12 min) to give compound (3R)-N-benzyl-2-hydroxy-3-octanamido-5-oxopyrrolidine-2-carboxamide (4 mg, 8.10 umol, 12.23% yield, 76% purity) as a white solid.

### Example 244: (R)-2-(3-cyclohexylpropanamido)-N′-(3,4-dihydroxyphenethyl)-N⁴-hydroxysuccinamide

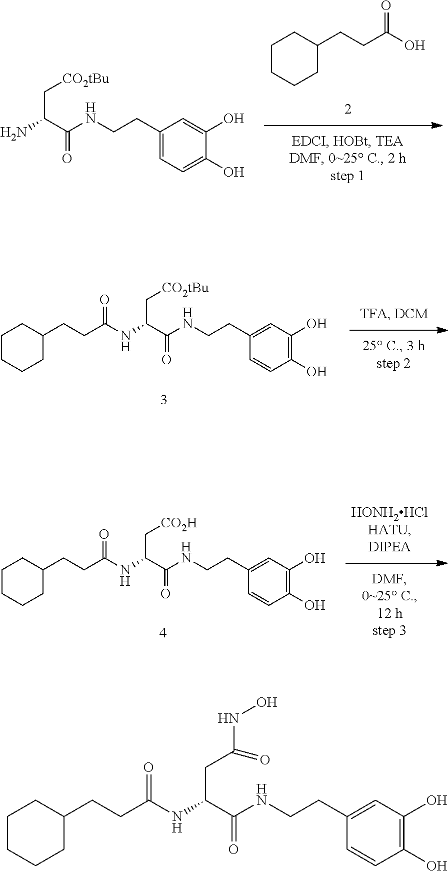 [SMILES: CC(C)(C)OC(=O)C[C@@H](N)C(=O)NCCc1ccc(O)c(O)c1 ; O=C(O)CCC1CCCCC1 ; CC(C)(C)OC(=O)C[C@@H](NC(=O)CCC1CCCCC1)C(=O)NCCc1ccc(O)c(O)c1 ; O=C(O)C[C@@H](NC(=O)CCC1CCCCC1)C(=O)NCCc1ccc(O)c(O)c1 ; O=C(C[C@@H](NC(=O)CCC1CCCCC1)C(=O)NCCc1ccc(O)c(O)c1)NO]

### Step 1

To a solution of tert-butyl (3R)-3-amino-4-[2-(3,4-dihydroxyphenyl)ethylamino]-4-oxo-butanoate (350 mg, 1.08 mmol, 1 eq) and 3-cyclohexylpropanoic acid (202.28 mg, 1.29 mmol, 221.80 uL, 1.2 eq) in DMF (5 mL) was added HOBt (174.96 mg, 1.29 mmol, 1.2 eq), EDCI (248.22 mg, 1.29 mmol, 1.2 eq) and TEA (218.37 mg, 2.16 mmol, 300.37 uL, 2 eq). The mixture was warmed to 25° C. and stirred for 2 hr. The reaction mixture was poured into H₂O (20 mL) slowly, filtered and filter cake concentrated under reduced pressure to give a crude product. The crude product was triturated with PE:EA=10:1 (22 mL) at 25° C. for 20 min. The mixture was filtered and filter cake concentrated under reduced pressure to give a residue. The residue was purified by column chromatography (SiO₂, Petroleum ether/Ethyl acetate=5/1 to 2/1) to give the compound tert-butyl (3R)-3-(3-cyclohexylpropanoylamino)-4-[2-(3,4-dihydroxyphenyl)ethylamino]-4-oxo-butanoate (400 mg, 864.72 umol, 80.14% yield) as a white solid.

### Step 2

To a solution of tert-butyl (3R)-3-(3-cyclohexylpropanoylamino)-4-[2-(3,4-dihydroxyphenyl) ethylamino]-4-oxo-butanoate (400 mg, 864.72 umol, 1 eq) in DCM (2 mL) was added TFA (3.08 g, 27.01 mmol, 2 mL, 1 eq). The mixture was stirred at 25° C. and for 3 hr. The reaction mixture was concentrated under reduced pressure to remove the solvent. The residue was purified by prep-TLC (SiO₂, DCM: MeOH=5:1) to give the compound (3R)-3-(3-cyclohexyl propanoylamino)-4-[2-(3,4-dihydroxyphenyl)ethylamino]-4-oxo-butanoic acid (150 mg, 369.03 umol) as a white solid.

### Step 3

To a solution of (3R)-3-(3-cyclohexylpropanoylamino)-4-[2-(3,4-dihydroxyphenyl)ethylamino]-4-oxo-butanoic acid (48 mg, 118.09 umol, 1 eq) and HATU (53.88 mg, 141.71 umol, 1.2 eq) in DMF (1.5 mL) was added dropwise DIPEA (30.52 mg, 236.18 umol, 41.14 uL, 2 eq) at 0° C. Then a mixture of DIPEA (30.52 mg, 236.18 umol, 41.14 uL, 2 eq) and hydroxylamine hydrochloride (16.41 mg, 236.18 umol, 2 eq) in DMF (0.5 mL) was added dropwise to the mixture at 0° C. The resulting mixture was stirred at 0° C. for 3 hr. The reaction mixture was poured into H₂O (10 mL) slowly, filtered and filter cake concentrated under reduced pressure to give a residue. The residue was purified by prep-HPLC (column: Phenomenex luna C18 80*40 mm*3 um; mobile phase: [water(0.04% HCl)-ACN]; B %: 23%-37%, 7 min) to give the compound (2R)-2-(3-cyclohexylpropanoylamino)-N-[2-(3,4-dihydroxyphenyl)ethyl]-4-(hydroxyamino)-4-oxo-butanamide (10 mg) as a white solid. LCMS: (M+H⁺): 422.2 @ 1.952 min.

### Example 245: (R)-N′-(4-hydroxyphenethyl)-N⁴-methoxy-2-octanamidosuccinamide

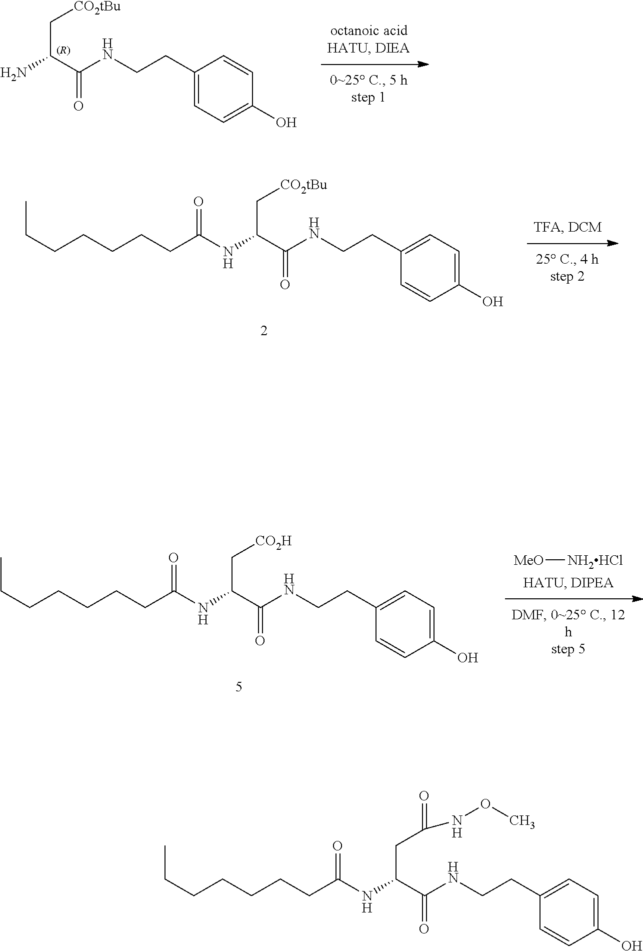

### Step 1

A mixture of tert-butyl (3R)-3-amino-4-[2-(4-hydroxyphenyl)ethylamino]-4-oxo-butanoate (0.9 g, 2.92 mmol, 1 eq), octanoic acid (420.89 mg, 2.92 mmol, 462.51 uL, 1 eq) in DMF (5 mL) was added HATU (1.33 g, 3.50 mmol, 1.2 eq), DIEA (829.83 mg, 6.42 mmol, 1.12 mL, 2.2 eq) at 0° C. and was degassed and purged with N2 for 3 times. The mixture was stirred at 25° C. for 5 hr under N₂ atmosphere. The combined organic phase was diluted with EtOAc 10 mL and washed with water 30 mL (10 mL*3) and brine 40 mL (20 mL*2), dried with anhydrous Na₂SO₄, filtered and concentrated under reduced pressure to give a residue. The residue was purified by column chromatography (SiO₂, Petroleum ether/Ethyl acetate=10/1 to 1/1) to give the compound tert-butyl (3R)-4-[2-(4-hydroxyphenyl)ethylamino]-3-(octanoylamino)-4-oxo-butanoate (1.3 g, 2.69 mmol, 92.25% yield, 90% purity) as white solid.

### Step 2

A mixture of tert-butyl (3R)-4-[2-(4-hydroxyphenyl)ethylamino]-3-(octanoylamino)-4-oxo-butanoate (0.3 g, 690.34 umol, 1 eq) in DCM (1 mL) was added TFA (1.54 g, 13.51 mmol, 1 mL, 19.56 eq) and was stirred at 25° C. for 4 hr under N2 atmosphere. The reaction mixture was concentrated under reduced pressure to give the compound (3R)-4-[2-(4-hydroxyphenyl)ethyl amino]-3-(octanoylamino)-4-oxo-butanoic acid (270 mg, crude) as white solid.

### Step 3

A mixture of O-methylhydroxylamine (101.51 mg, 1.22 mmol, 2 eq, HCl), DIEA (157.09 mg, 1.22 mmol, 211.71 uL, 2 eq) in DMF (1 mL) was added to (3R)-4-[2-(4-hydroxyphenyl) ethylamino]-3-(octanoylamino)-4-oxo-butanoic acid (230 mg, 607.72 umol, 1 eq), DIEA (157.09 mg, 1.22 mmol, 211.71 uL, 2 eq) and HATU (254.18 mg, 668.50 umol, 1.1 eq) in DMF (2 mL) at 0° C. and the mixture was stirred at 25° C. for 12 hr under N₂ atmosphere. The reaction mixture was poured into ACN 5 mL and filtered, the filtrate was concentrated under reduced pressure to give a residue. The residue was purified by prep-HPLC (column: Welch Xtimate C18 100*25 mm*3 um; mobile phase: [water(0.05% HCl)-ACN]; B %: 20%-50%, 8 min) to give the compound (2R)-N-[2-(4-hydroxyphenyl)ethyl]-N′-methoxy-2-(octanoylamino)butanediamide (100 mg, 233.13 umol, 38.36% yield, 95% purity) as white solid.

### Example 246: (R)-N′-((S)-2-(4-hydroxyphenyl)propyl)-2-octanamidosuccinamide &

(R)-N′-((R)-2-(4-hydroxyphenyl)propyl)-2-octanamidosuccinamide

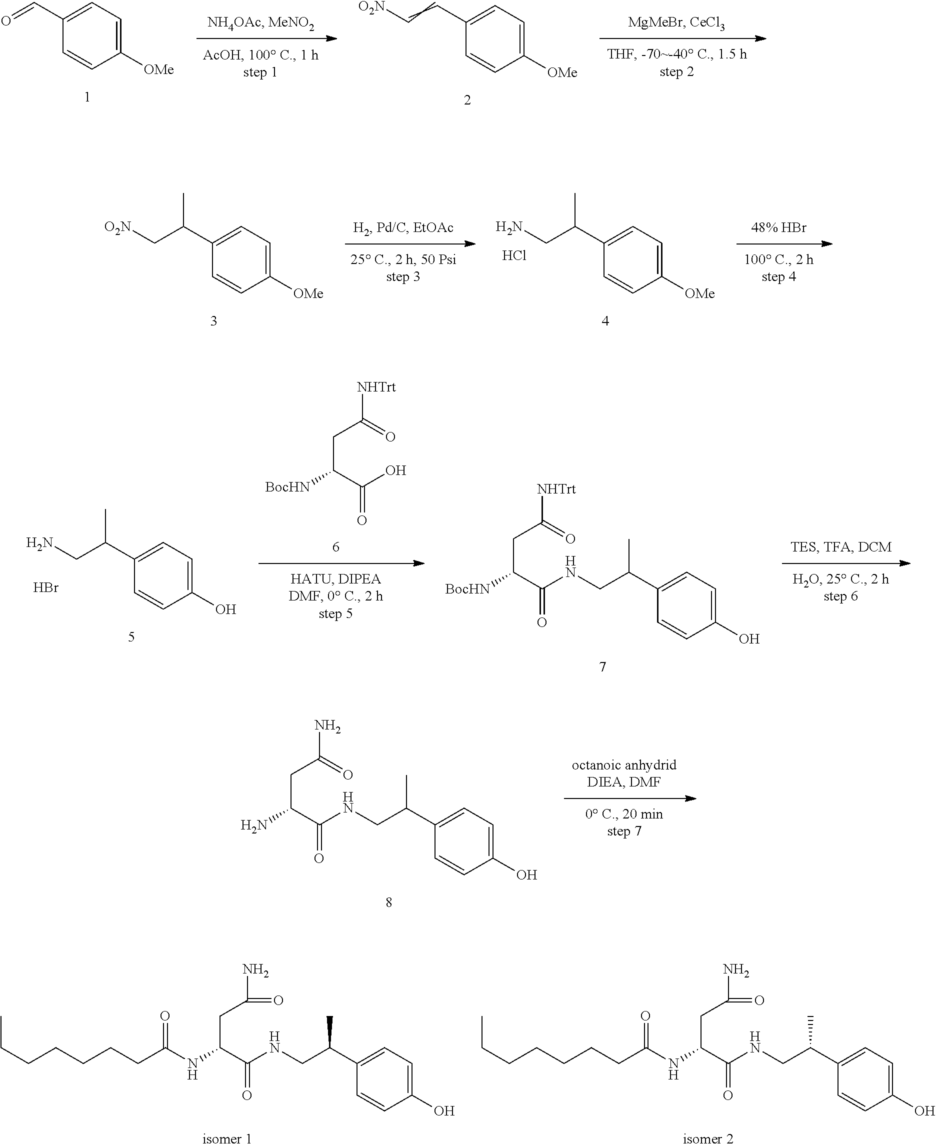

### Step 1

To a solution of 4-methoxybenzaldehyde (10 g, 73.45 mmol, 8.93 mL, 1 eq) in AcOH (100 mL) was added NH₄OAc (9.62 g, 124.87 mmol, 1.7 eq) and nitromethane (51.51 g, 843.87 mmol, 45.58 mL, 11.49 eq), stirred at 100° C. for 1 h. The mixture was added into H₂O (400 mL), filtered and the solid was concentrated under reduced pressure to give the 1-methoxy-4-(2-nitrovinyl) benzene (8 g, crude) as a yellow solid.

### Step 2

a solution of CeCl₃ (5.50 g, 22.32 mmol, 1.40 mL, 2 eq) in THF (30 mL) was stirred at −70° C. for 20 min, then MeMgBr (3 M, 7.44 mL, 2 eq) was added into the mixture and stirred at −70° C. for 1 h. The reaction mixture was warmed to −40° C. To the mixture was added a solution of 1-methoxy-4-(2-nitrovinyl)benzene (2 g, 11.16 mmol, 1 eq) in THF (40 mL) and stirred at −40° C. for 10 min. The reaction was quenched with acetic acid (10 mL) and diluted with water (50 mL) and extracted with MTBE (20 mL*3). The organic extracts were washed with brine (50 mL*3), dried with Na₂SO₄ and filtered. The filtrate was concentrated under reduced pressure. The residue was purified by column chromatography (SiO₂, Petroleum ether/Ethyl acetate=100/1 to 50/1) to give the 1-methoxy-4-(1-methyl-2-nitro-ethyl)benzene (1 g, 5.12 mmol, 45.89% yield) as colorless oil.

### Step 3

The solution of 10% Pd/C (300 mg, 1.00 eq) in EtOAc (20 mL) was added 1-methoxy-4-(1-methyl-2-nitro-ethyl)benzene (1 g, 5.12 mmol, 1 eq) and stirred at 25° C. for 2 h under 50 Psi under H₂. The mixture was filtered and the filtrate was concentrated under reduced pressure. 2-(4-methoxyphenyl)propan-1-amine (700 mg, crude) was obtained as colorless oil.

### Step 4

A solution of 2-(4-methoxyphenyl)propan-1-amine (200 mg, 1.21 mmol, 1 eq) in HBr (3.10 g, 18.42 mmol, 2.08 mL, 48% purity, 15.21 eq) was stirred at 100° C. for 2 h. The mixture was concentrated under reduced pressure to remove the solvent. The residue was diluted with water (50 mL) and extracted with EtOAc (2 mL*3), then water phase was concentrated under reduced pressure to give the compound 4-(2-amino-1-methyl-ethyl)phenol (260 mg, crude, HBr) as a brown solid.

### Step 5

To a solution of (2R)-2-(tert-butoxycarbonylamino)-4-oxo-4-(tritylamino)butanoic acid (150 mg, 316.09 umol, 1 eq) and 4-(2-amino-1-methyl-ethyl)phenol (86.67 mg, 373.37 umol, 1.18 eq, HBr) in DMF (5 mL) was added DIEA (102.13 mg, 790.23 umol, 137.64 uL, 2.5 eq) and HATU (180.28 mg, 474.14 umol, 1.5 eq) at 0° C. and stirred at 0° C. for 2 h. The mixture was added into H₂O (15 mL), filtered and the solid was concentrated under reduced pressure to give the compound tert-butyl N-[(1R)-1-[2-(4-hydroxyphenyl) propylcarbamoyl]-3-oxo-3-(tritylamino) propyl]carbamate (600 mg, crude) as a white solid.

### Step 6

A solution of tert-butyl N-[(1R)-1-[2-(4-hydroxyphenyl)propylcarbamoyl]-3-oxo-3-(tritylamino)propyl]carbamate (100 mg, 164.54 umol, 1 eq) in TES (0.5 mL), DCM (1 mL), TFA (18 mL), H₂O (0.5 mL) was stirred at 25° C. for 2 h. The mixture was filtered and the filtrate concentrated under reduced pressure to give the compound (2R)-2-amino-N-[2-(4-hydroxyphenyl)propyl]butanediamide (261 mg, crude) as a yellow solid.

### Step 7

To a solution of (2R)-2-amino-N-[2-(4-hydroxyphenyl)propyl]butanediamide (130 mg, 490.00 umol, 1 eq) and octanoyl octanoate (106.00 mg, 392.00 umol, 0.8 eq) in DMF (5 mL) was added DIEA (126.65 mg, 979.99 umol, 170.69 uL, 2 eq) at 0° C. and stirred at 0° C. for 20 min. The mixture was concentrated under reduced pressure. The residue was purified by prep-HPLC(column: Welch Xtimate C18 100*25 mm*3 um; mobile phase: [water(0.05% HCl)-ACN]; B %: 15%-45%, 8 min) followed by SFC (column: DAICEL CHIRALPAK AD (250 mm*30 mm, 10 um); mobile phase: [0.10% NH₃H₂O ETOH]; B %: 35%-35%, 6 min). The compounds isomer 1 and isomer 2 were obtained as white solids.

### Example 247: (3R)-N-benzyl-3-decanamido-2-hydroxy-5-oxopyrrolidine-2-carboxamide

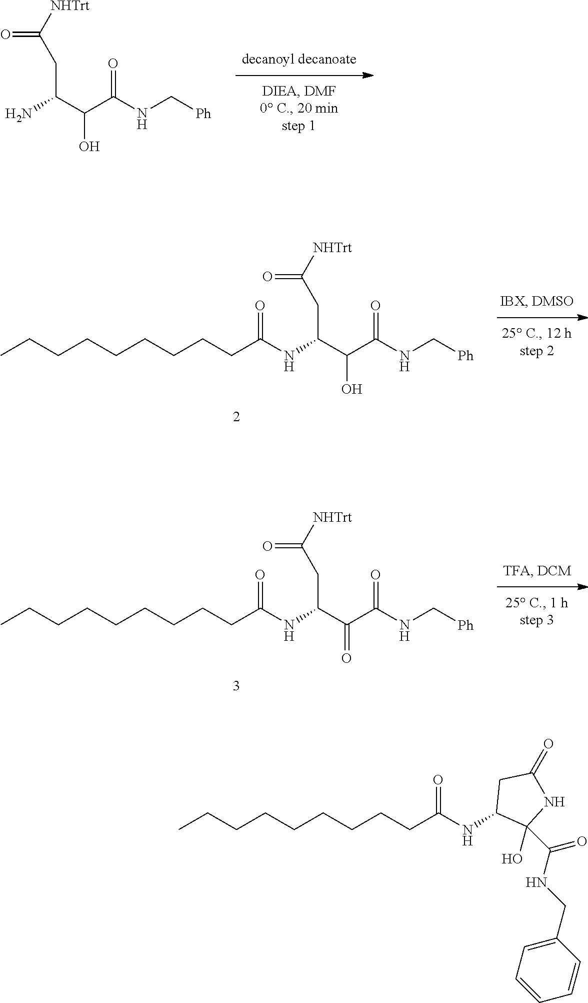

### Step 1

To a solution of (3R)-3-amino-N-benzyl-2-hydroxy-N′-trityl-pentanediamide (600 mg, 1.22 mmol, 1 eq) in DMF (5 mL) was added decanoyl decanoate (396.90 mg, 1.22 mmol, 1 eq) and DIEA (188.52 mg, 1.46 mmol, 254.07 uL, 1.2 eq) at 0° C., then stirred at 0° C. for 20 min. The mixture was concentrated under reduced pressure. The residue was purified by column chromatography (SiO₂, Petroleum ether/Ethyl acetate=10/1 to 1/1) to give the compound (3R)-N-benzyl-3-(decanoylamino)-2-hydroxy-N′-trityl-pentanediamide (500 mg, 50% yield) as a white solid.

### Step 2

To a solution of (3R)-N-benzyl-3-(decanoylamino)-2-hydroxy-N′-trityl-pentanediamide (300 mg, 463.07 umol, 1 eq) in DMSO (5 mL) was added IBX (389.01 mg, 1.39 mmol, 3 eq), and stirred at 25° C. for 12 h. TLC showed the starting reactant consumed. The mixture was added into H₂O (10 mL), the solid was formed and filtered. The solid was concentrated under reduced pressure. The residue was purified by column chromatography (SiO₂, Petroleum ether/Ethyl acetate=10/1 to 1/1). (3R)-N-benzyl-3-(decanoylamino)-2-oxo-N′-trityl-pentanediamide (140 mg, 216.78 umol, 46.81% yield) was obtained as a yellow solid.

### Step 3

To a solution of (3R)-N-benzyl-3-(decanoylamino)-2-oxo-N′-trityl-pentanediamide (30 mg, 46.45 umol, 1 eq) in DCM (2.5 mL) was added TFA (463.43 mg, 4.06 mmol, 300.93 uL, 87.50 eq) and stirred at 25° C. for 1 h. The mixture was concentrated under reduced pressure. The residue was purified by prep-HPLC(column: Phenomenex Luna C18 100*30 mm*5 um; mobile phase: [water(0.04% HCl)-ACN]; B %: 30%-60%, 10 min) to give the compound (2S,3R)-N-benzyl-3-(decanoylamino)-2-hydroxy-5-oxo-pyrrolidine-2-carboxamide (5.7 mg, 11.16 umol, 24.02% yield, 79% purity) as a white solid.

### Example 248: (3S)-N-benzyl-2-hydroxy-3-octanamido-5-oxopyrrolidine-2-carboxamide

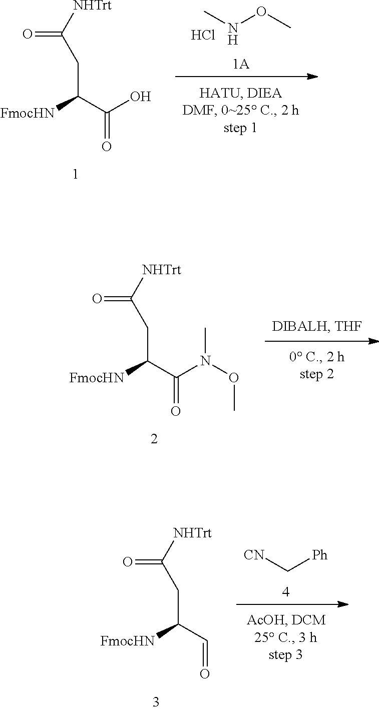

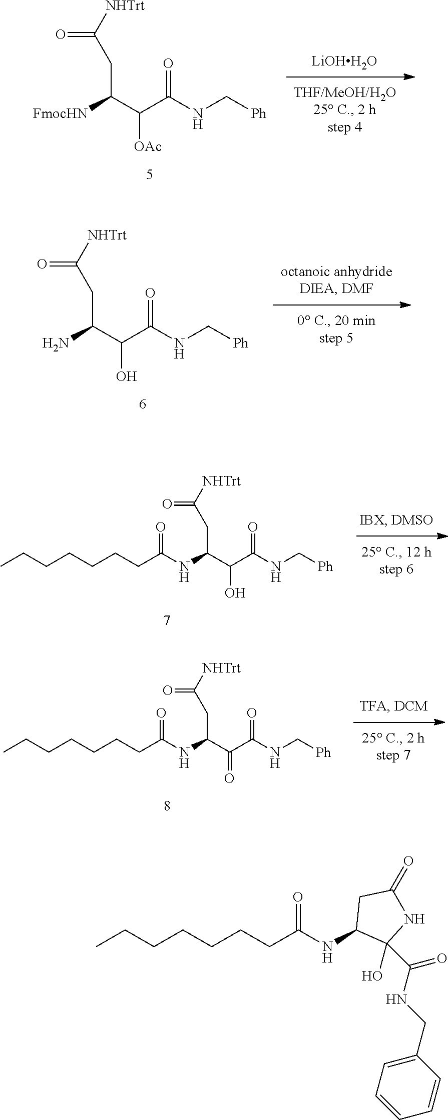

### Step 1

To a solution of N-methoxymethanamine (767.80 mg, 12.57 mmol, 1.5 eq) and (2S)-2-(9H-fluoren-9-ylmethoxycarbonylamino)-4-oxo-4-(tritylamino)butanoic acid (5.00 g, 8.38 mmol, 1 eq) in DMF (50 mL) was added HATU (3.82 g, 10.06 mmol, 1.2 eq) and DIPEA (3.25 g, 25.14 mmol, 4.38 mL, 3 eq) at 0° C. The mixture was stirred at 25° C. for 2 h. The mixture was poured into H₂O (100 mL). The solid was formed, and the solid was filtered and concentrated the filter cake under reduced pressure to give the compound 9H-fluoren-9-ylmethyl N-[(1S)-1-[methoxy(methyl)carbamoyl]-3-oxo-3-(trityl amino)propyl]carbamate (5 g, crude) as a brown solid.

### Step 2

To a solution of 9H-fluoren-9-ylmethyl N-[1-[methoxy(methyl)carbamoyl]-3-oxo-3-(tritylamino) propyl]carbamate (1 g, 1.56 mmol, 1 eq) in THF (20 mL) was added DIBAL-H (1 M, 3.13 mL, 2 eq) at 0° C. The mixture was stirred at 0° C. for 2 h. TLC showed the starting reactant consumed. The mixture was poured into H₂O (10 mL), then extracted with EtOAc (10 mL*3), combined organic layer was washed with brine (10 mL*2), dried over Na₂SO₄, filtered and the filtrate was concentrated under reduced pressure. The residue was purified by column chromatography (SiO₂, Petroleum ether/Ethyl acetate=10/1 to 1/1) to give the 9H-fluoren-9-ylmethyl N-[1-formyl-3-oxo-3-(tritylamino)propyl]carbamate (300 mg, 516.64 umol, 33.05% yield) as a white solid.

### Steps 3 and 4

To a solution of 9H-fluoren-9-ylmethyl N-[1-formyl-3-oxo-3-(tritylamino)propyl]carbamate (3.6 g, 6.20 mmol, 1 eq) in DCM (40 mL) was added AcOH (744.64 mg, 12.40 mmol, 709.19 uL, 2 eq) and isocyanomethylbenzene (798.95 mg, 6.82 mmol, 830.51 uL, 1.1 eq). The mixture was stirred at 25° C. for 3 h. The mixture was concentrated under reduced pressure to get a residue. The residue was dissolved with THF (40 mL) and MeOH (15 mL). A solution of LiOH·H₂O (520.28 mg, 12.40 mmol, 2 eq) in H₂O (5 mL) was added into the mixture and stirred at 25° C. for 2 h. The mixture was concentrated under reduced pressure to remove the solvent, then the residue was added H₂O (10 mL) and extracted with EtOAc (10 mL*3), combined organic layer was washed with brine (10 mL*2), dried over Na₂SO₄, filtered and the filtrate was concentrated under reduced pressure to give the crude product. The crude product was triturated with PE/EA=10/1 (20 mL) at 25° C. for 30 min, then the solid was filtered and the filtrate was concentrated under reduced pressure to give (3S)-3-amino-N-benzyl-2-hydroxy-N′-trityl-pentanediamide (2 g, 4.05 mmol, 65.35% yield) as a yellow solid.

### Step 5

To a solution of (3S)-3-amino-N-benzyl-2-hydroxy-N′-trityl-pentanediamide (1 g, 2.03 mmol, 1 eq) in DMF (5 mL) was added octanoyl octanoate (548.93 mg, 2.03 mmol, 1 eq) and DIEA (314.84 mg, 2.44 mmol, 424.31 uL, 1.2 eq) at 0° C. and stirred for 20 min. The mixture was concentrated under reduced pressure. The residue was purified by column chromatography (SiO₂, Petroleum ether/Ethyl acetate=10/1 to 0/1) to give the compound (3S)-N-benzyl-2-hydroxy-3-(octanoylamino)-N′-trityl-pentanediamide (500 mg, 806.72 umol, 39.74% yield) as a yellow solid.

### Step 6

To a solution of (3S)-N-benzyl-2-hydroxy-3-(octanoylamino)-N′-trityl-pentanediamide (500 mg, 806.72 umol, 1 eq) in DMSO (5 mL) was added IBX (677.69 mg, 2.42 mmol, 3 eq), and stirred at 25° C. for 12 h. The mixture was added H₂O (10 mL) and extracted with EtOAc (5 mL*3), combined organic layer was washed with brine (5 mL*2), dried over Na₂SO₄, filtered. The filtrate was concentrated under reduced pressure. The residue was purified by column chromatography (SiO₂, Petroleum ether/Ethyl acetate=10/1 to 0/1) to give the compound (3S)-N-benzyl-3-(octanoylamino)-2-oxo-N′-trityl-pentanediamide (200 mg, 323.74 umol, 40.13% yield) as a yellow solid.

### Step 7

To a solution of (3S)-N-benzyl-3-(octanoylamino)-2-oxo-N′-trityl-pentanediamide (50 mg, 80.94 umol, 1 eq) in DCM (2.5 mL) was added TFA (770.00 mg, 6.75 mmol, 500.00 uL, 83.44 eq). The mixture was stirred at 25° C. for 2 h. The mixture was concentrated under reduced pressure. The residue was purified by prep-HPLC (column: Phenomenex Luna C18 100*30 mm*5 um; mobile phase: [water(0.04% HCl)-ACN]; B %: 30%-60%, 10 min) to give the compound (3S)-N-benzyl-2-hydroxy-3-(octanoylamino)-5-oxo-pyrrolidine-2-carboxamide (12 mg, 28.13 umol, 34.75% yield) as a white solid.

### Example 249: (3R)-N-butyl-2-hydroxy-3-octanamido-5-oxopyrrolidine-2-carboxamide

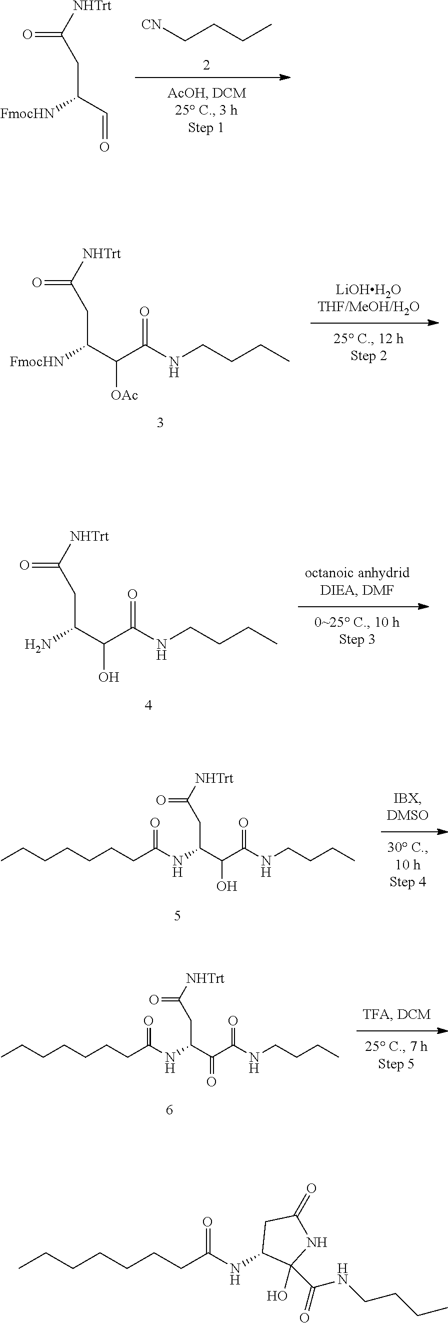

### Step 1

To a solution of 9H-fluoren-9-ylmethyl N-[(1R)-1-formyl-3-oxo-3-(tritylamino)propyl]carbamate (3.5 g, 6.03 mmol, 1 eq) in DCM (20 mL) was added AcOH (1.09 g, 18.08 mmol, 1.03 mL, 3 eq) and 1-isocyanobutane (501.08 mg, 6.03 mmol, 630.28 uL, 1 eq). The mixture was stirred at 25° C. for 3 hr. The reaction mixture was concentrated under reduced pressure to give a residue and the residue was dissolved in EtOAc. The organic phase was washed with 1N HCl, 5% NaHCO₃ and brine, dried over Na₂SO₄ and the filtrate was concentrated under reduced pressure to give a residue. The residue was purified by column chromatography (SiO₂, Petroleum ether/Ethyl acetate=1/0 to 0/1) to give the compound [(2R)-1-(butylcarbamoyl)-2-(9H-fluoren-9-ylmethoxycarbonylamino)-4-oxo-4-(tritylamino)butyl]acetate (1.6 g, 2.21 mmol, 36.67% yield) as a white solid.

### Step 2

To a solution of [(2R)-1-(butylcarbamoyl)-2-(9H-fluoren-9-ylmethoxycarbonylamino)-4-oxo-4-(tritylamino)butyl]acetate (1.6 g, 2.21 mmol, 1 eq) in THF (16 mL) and MeOH (6 mL) was added LiOH·H₂O (185.51 mg, 4.42 mmol, 2 eq) in H₂O (1.5 mL). The mixture was stirred at 25° C. for 12 hr. The reaction mixture was concentrated under reduced pressure to remove the solvent. The residue was diluted with water 7 mL and extracted with EtOAc (30 mL, 10 mL*3), dried over Na₂SO₄, filtered and the filtrate concentrated under reduced pressure to give a residue. The residue was purified by column chromatography (SiO₂, Petroleum ether/Ethyl acetate=1/0 to 0/1) to get (3R)-3-amino-N-butyl-2-hydroxy-N′-trityl-pentanediamide (0.8 g, 1.74 mmol, 78.75% yield) as a white solid.

### Step 3

To a solution of (3R)-3-amino-N-butyl-2-hydroxy-N′-trityl-pentanediamide (200 mg, 435.18 umol, 1 eq) in DMF (10 mL) was added octanoyl octanoate (105.91 mg, 391.66 umol, 0.9 eq) and DIEA (168.73 mg, 1.31 mmol, 227.40 uL, 3 eq) at 0° C. The mixture was stirred at 25° C. for 10 hr. The mixture was poured into H₂O (30 mL) at 0° C., then extracted with EtOAc (15 mL*3), combined organic layer was washed with brine (10 mL*2), dried over Na₂SO₄, filtered and concentrated. The filtrate under reduced pressure to give a residue. The residue was purified by column chromatography (SiO₂, Petroleum ether/Ethyl acetate=1/0 to 0/1) to get (3R)-N-butyl-2-hydroxy-3-(octanoylamino)-N′-trityl-pentanediamide (80 mg, 136.57 umol, 31.38% yield) as a white solid.

### Step 4

To a solution of (3R)-N-butyl-2-hydroxy-3-(octanoylamino)-N′-trityl-pentanediamide (80 mg, 136.57 umol, 1 eq) in DMSO (2 mL) was added IBX (114.73 mg, 409.71 umol, 3 eq). The mixture was stirred at 30° C. for 10 hr. The mixture was poured into H₂O (7 mL) at 0° C. and extracted with DCM (15 mL*3), dried over Na₂SO₄, filtered and the filtrate was concentrated under reduced pressure to give a residue. The residue was purified by column chromatography (SiO₂, Petroleum ether/Ethyl acetate=1/0 to 0/1) to give the compound (3R)-N-butyl-3-(octanoylamino)-2-oxo-N′-trityl-pentanediamide (25 mg, 28.27 umol, 20.70% yield, 66% purity) as a white solid.

### Step 5

A mixture of (3R)-N-butyl-3-(octanoylamino)-2-oxo-N′-trityl-pentanediamide (25 mg, 42.83 umol, 1 eq) in DCM (1 mL) and TFA (0.2 mL) was stirred at 25° C. for 7 hr under N2 atmosphere. The reaction mixture was concentrated under reduced pressure to give a residue. The residue was purified by prep-HPLC (column: Phenomenex Luna Cis 100*30 mm*5 um; mobile phase: [water(0.04% HCl)-ACN]; B %: 40%-70%, 10 min) to give the compound (3R)-N-butyl-2-hydroxy-3-(octanoylamino)-5-oxo-pyrrolidine-2-carboxamide (2.2 mg, 5.67 umol, 13.24% yield, 88% purity) as a white solid.

### Example 250: (R)-2-(6-(benzyl(methyl)amino)hexanamido)-N′-(4-hydroxyphenethyl)succinamide

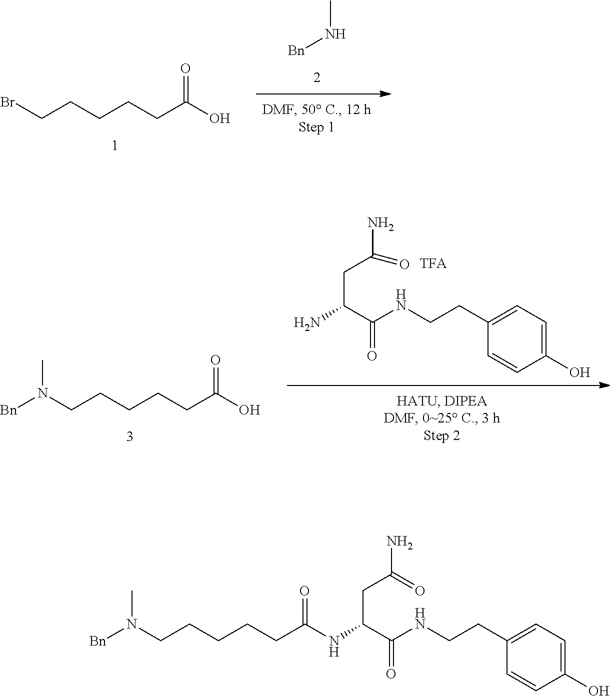

### Step 1

To a solution of 6-bromohexanoic acid (1 g, 5.13 mmol, 1 eq) in DMF (10 mL) was added N-methyl-1-phenyl-methanamine (3.11 g, 25.63 mmol, 3.31 mL, 5 eq). The mixture was stirred at 50° C. for 12 hr. The reaction mixture concentrated under reduced pressure to give a residue. The residue was purified by column chromatography (SiO₂, Petroleum ether/Ethyl acetate=1/1 to Ethyl acetate/MeOH=0/1) to give the compound 6-[benzyl(methyl)amino]hexanoic acid (800 mg, 3.40 mmol, 66.31% yield) as yellow oil.

### Step 2

To a solution of 6-[benzyl(methyl)amino]hexanoic acid (100 mg, 424.95 umol, 1 eq) and (2R)-2-amino-N-[2-(4-hydroxyphenyl)ethyl]butanediamide (201.81 mg, 552.44 umol, 1.3 eq, TFA) in DMF (5 mL) was added HATU (193.89 mg, 509.94 umol, 1.2 eq) and DIEA (137.30 mg, 1.06 mmol, 185.05 uL, 2.5 eq) at 0° C. The mixture was stirred at 25° C. for 3 hr. The reaction mixture was concentrated under reduced pressure to give a residue. The residue was purified by column chromatography (SiO₂, Petroleum ether/Ethyl acetate=0/1 to Ethyl acetate/MeOH=0/1) and prep-HPLC ([water (10 mM NH₄HCO₃)-ACN]) to give the compound (2R)-2-[6-[benzyl (methyl)amino]hexanoylamino]-N-[2-(4-hydroxyphenyl) ethyl]butanediamide (12 mg, 25.61 umol, 6.03% yield) as white solid.

### Example 251: (R)-N′-(4-hydroxyphenethyl)-2-(6,6,6-trifluorohexanamido)succinamide

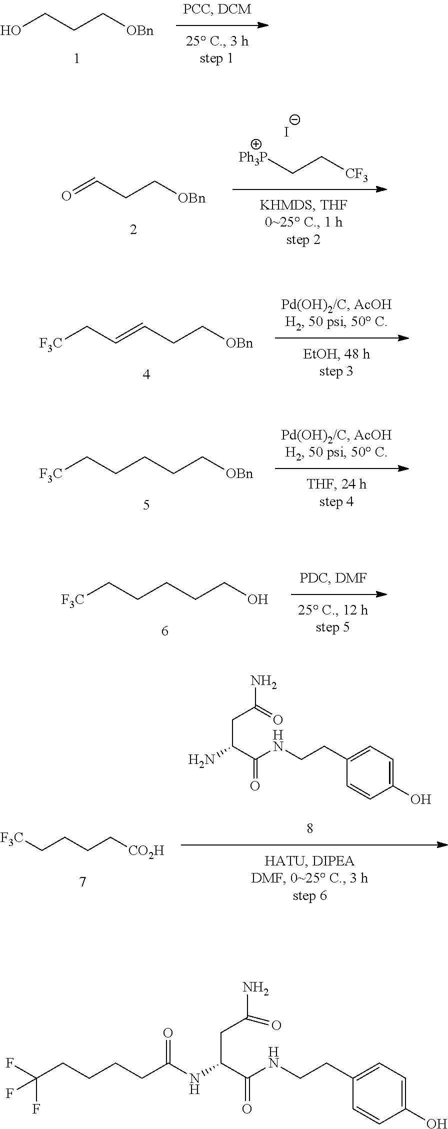

### Step 1

To a mixture of 3-benzyloxypropan-1-ol (5 g, 30.08 mmol, 4.76 mL, 1 eq) in DCM (1250 mL) was added PCC (9.86 g, 45.72 mmol, 1.52 eq) and the mixture was stirred at 25° C. for 3 hr. The reaction mixture was treated with MTBE (1000 mL), then filtered through a pad of celite and silica gel (1:1). The filtrate was concentrated in vacuum to get compound 3-benzyloxypropanal (5 g, crude) as brown oil.

### Step 2

To a mixture of triphenyl(3,3,3-trifluoropropyl)phosphonium iodide (19.25 g, 39.59 mmol, 1.3 eq) in THF (225 mL) was added KHMDS (1 M, 38.06 mL, 1.25 eq) dropwise at 0° C. under N₂. The mixture changed from white to orange to green and it was stirred at 25° C. for 30 min. Then the mixture was cooled to 0° C. and a solution of 3-benzyloxypropanal (5 g, 30.45 mmol, 1 eq) in THF (25 mL) was added dropwise at 0° C. The mixture was stirred at 0° C. for 30 min. The mixture was quenched by sat. NH₄Cl (200 mL) and the mixture was extracted with ethyl acetate (120 mL*2). The combined organic phase was washed with brine (60 mL), dried with anhydrous Na₂SO₄, filtered and the filtrate was concentrated in vacuum to get a residue. The residue was purified by flash silica gel chromatography (Silica Flash Column, Eluent of 0-20% Ethyl acetate/Petroleum ethergradient) to give the compound [(E)-6,6,6-trifluorohex-3-enoxy]methylbenzene (3 g, crude) as yellow oil.

### Step 3

To a solution of [(E)-6,6,6-trifluorohex-3-enoxy]methylbenzene (3.9 g, 15.97 mmol, 1 eq) in EtOH (160 mL) was added 10% Pd(OH)₂/C (100 mg, 15.97 mmol, 1.00 eq) and AcOH (1 mL) under N₂. The suspension was degassed under vacuum and purged with H₂ several times. The mixture was stirred under H₂ (50 psi) at 50° C. for 48 hours. The mixture is filtered through celite and the solvent is removed under reduced pressure to give a residue. The residue was purified by column chromatography (SiO₂, Petroleum ether/Ethyl acetate=20/1 to 10/1) to give the compound 6,6,6-trifluorohexoxymethylbenzene (2.1 g, 8.53 mmol, 53.41% yield) as colorless liquid.

### Step 4

To a solution of 6,6,6-trifluorohexoxymethylbenzene (2.1 g, 8.53 mmol, 1 eq) in THF (200 mL) was added 10% Pd(OH)₂/C (2 g, 8.53 mmol, 1.00 eq) and AcOH (1 mL) under N2. The suspension was degassed under vacuum and purged with H₂ several times. The mixture was stirred under H₂ (50 psi) at 50° C. for 24 hours. The mixture is filtered through celite and the solvent is removed under reduced pressure to give the compound 6,6,6-trifluorohexan-1-ol (3 g, crude) as white solid.

### Step 5

To a solution of 6,6,6-trifluorohexan-1-ol (2.3 g, 14.73 mmol, 1 eq) in DMF (2 mL) was added PDC (16.62 g, 44.19 mmol, 3 eq) and stirred at 25° C. for 12 h. The reaction mixture was diluted with H₂O (200 mL) and extracted with EtOAc (600 mL, 200 mL*3). The combined organic layers were dried over Na₂SO₄, filtered and concentrated under reduced pressure to give the compound 6,6,6-trifluorohexanoic acid (1.5 g, 8.82 mmol, 59.86% yield) as yellow liquid.

### Step 6

A mixture of 6,6,6-trifluorohexanoic acid (400 mg, 2.35 mmol, 1 eq), (2R)-2-amino-N-[2-(4-hydroxyphenyl)ethyl]butanediamide (1.30 g, 2.82 mmol, 79% purity, 1.20 eq, TFA), HATU (1.07 g, 2.82 mmol, 1.2 eq) in DMF (3 mL) was added DIEA (668.50 mg, 5.17 mmol, 900.94 uL, 2.2 eq) at 0° C. and was degassed and purged with N₂ for 3 times. The mixture was stirred at 25° C. for 5 hr under N₂ atmosphere. The mixture was diluted with ACN (3 mL) and filtered. The filtrate was concentrated under reduced pressure to give a residue. The residue was purified by prep-HPLC (column: Waters Xbridge Prep OBD C18 150*40 mm*10 um; mobile phase: [water(10 mM NH4HCO3)-ACN]; B %: 10%-30%, 8 min) to give the compound (2R)-N-[2-(4-hydroxyphenyl)ethyl]-2-(6,6,6-trifluorohexanoylamino)butanediamide (100 mg, 245.42 umol, 10.44% yield, 99% purity) as white solid.

### Example 252: (R)-N′-(3,4-dihydroxyphenethyl)-N⁴-hydroxy-2-octanamidosuccinamide

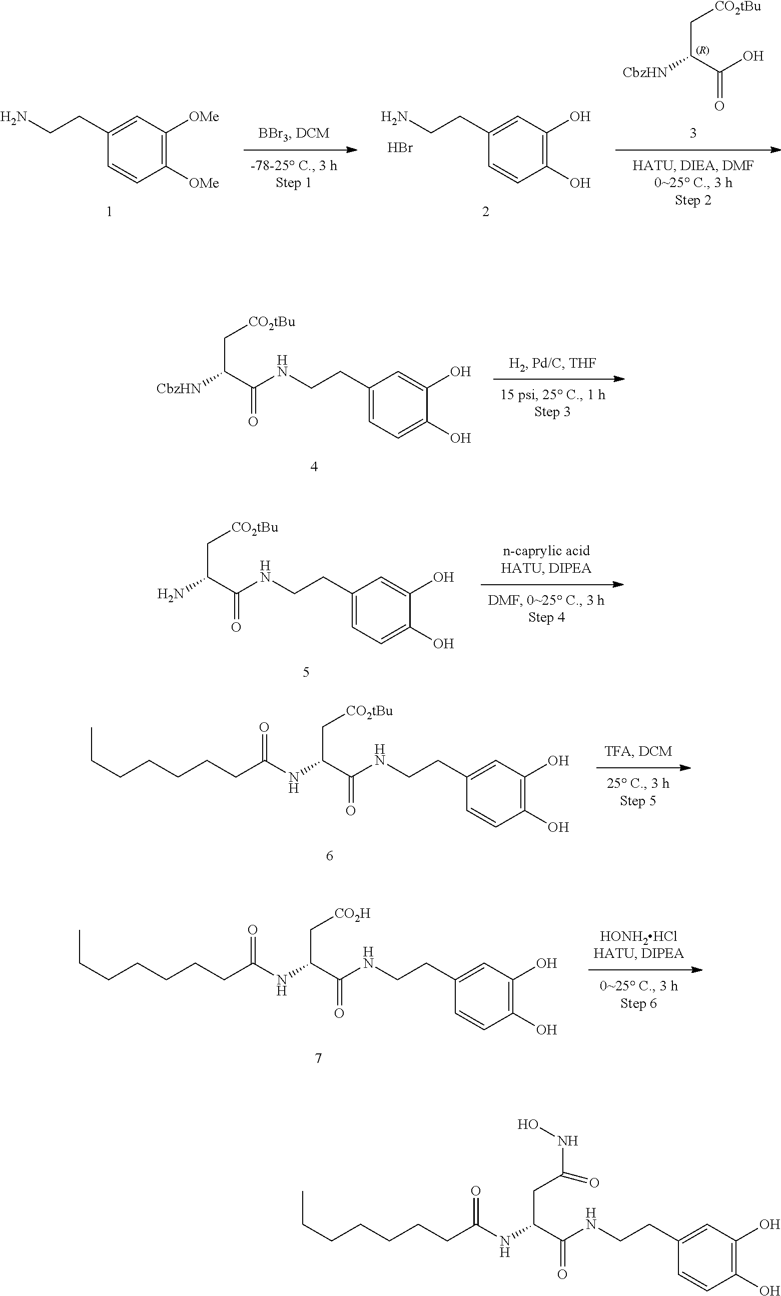

### Step 1

To a solution of 2-(3,4-dimethoxyphenyl)ethanamine (10 g, 55.18 mmol, 9.17 mL, 1 eq) in DCM (100 mL) was added BBr₃ (41.47 g, 165.53 mmol, 15.95 mL, 3 eq) at −78° C. The mixture was stirred at 25° C. for 3 hr. The reaction mixture was quenched by H₂O 100 mL at −78° C., and then extracted with DCM 150 mL (50 mL*3). The combined water layers were concentrated under reduced pressure to give a the compound 4-(2-aminoethyl)benzene-1,2-diol (10 g, 37.59 mmol, 68.13% yield, 88% purity, HBr) as brown solid.

### Step 2

To a solution of (2R)-2-(benzyloxycarbonylamino)-4-tert-butoxy-4-oxo-butanoic acid (5 g, 15.46 mmol, 1 eq) and 4-(2-aminoethyl)benzene-1,2-diol (5 g, 21.36 mmol, 1.38 eq, HBr) in DMF (50 mL) was added HATU (7.06 g, 18.56 mmol, 1.2 eq) and DIPEA (6.00 g, 46.39 mmol, 8.08 mL, 3 eq) at 0° C. The mixture was stirred at 25° C. for 3 hr. The reaction mixture was concentrated under reduced pressure to give a residue. The residue was purified by column chromatography (SiO₂, Petroleum ether/Ethyl acetate=10/1 to 1/1) to give the compound tert-butyl (3R)-3-(benzyloxycarbonylamino)-4-[2-(3,4-dihydroxyphenyl)ethylamino]-4-oxo-butanoate (5 g, 9.16 mmol, 52.50% yield, 84% purity) as colorless oil.

### Step 3

To a solution of tert-butyl (3R)-3-(benzyloxycarbonylamino)-4-[2-(3,4-dihydroxyphenyl) ethylamino]-4-oxo-butanoate (5 g, 10.91 mmol, 1 eq) in THF (30 mL) was added 10% Pd/C (3 g, 10.91 mmol,) and the mixture was stirred under H₂ (15 Psi.) at 25° C. for 1 hr. The reaction mixture was filtered and the filtrate was concentrated under reduced pressure to give the product tert-butyl (3R)-3-amino-4-[2-(3,4-dihydroxyphenyl)ethylamino]-4-oxo-butanoate (3 g, 9.25 mmol, 84.81% yield) as brown solid.

### Step 4

To a solution of octanoic acid (200 mg, 1.39 mmol, 219.78 uL, 1 eq) and tert-butyl (3R)-3-amino-4-[2-(3,4-dihydroxyphenyl)ethylamino]-4-oxo-butanoate (541.05 mg, 1.67 mmol, 1.2 eq) in DMF (10 mL) was added HATU (634.22 mg, 1.67 mmol, 1.2 eq) and DIPEA (395.22 mg, 3.06 mmol, 532.64 uL, 2.2 eq) at 0° C. The mixture was stirred at 25° C. for 3 hr. The reaction mixture was concentrated under reduced pressure to give a residue. The residue was purified by column chromatography (SiO₂, Petroleum ether/Ethyl acetate=10/1 to 1/1) to give the compound tert-butyl (3R)-4-[2-(3,4-dihydroxyphenyl)ethylamino]-3-(octanoylamino)-4-oxo-butanoate (500 mg, 887.77 umol, 40.00% yield, 80% purity) as colorless oil.

### Step 5

To a solution of tert-butyl (3R)-4-[2-(3,4-dihydroxyphenyl)ethylamino]-3-(octanoylamino)-4-oxo-butanoate (250 mg, 554.86 umol, 1 eq) in DCM (3 mL) was added TFA (4.62 g, 40.52 mmol, 3 mL, 73.02 eq). The mixture was stirred at 25° C. for 3 hr. The reaction mixture was concentrated under reduced pressure to give the product (3R)-4-[2-(3,4-dihydroxyphenyl)ethylamino]-3-(octanoylamino)-4-oxo-butanoic acid (200 mg, crude) as yellow oil.

### Step 6

To a solution of (3R)-4-[2-(3,4-dihydroxyphenyl)ethylamino]-3-(octanoylamino)-4-oxo-butanoic acid (200 mg, 507.02 umol, 1 eq) and hydroxylamine; hydrochloride (70.47 mg, 1.01 mmol, 2 eq) in DMF (3 mL) was added HATU (231.34 mg, 608.43 umol, 1.2 eq) and DIPEA (131.06 mg, 1.01 mmol, 176.63 uL, 2 eq) at 0° C. The mixture was stirred at 25° C. for 3 hr. The reaction mixture was concentrated under reduced pressure to give a residue. The residue was purified by prep-HPLC ([water (0.05% HCl)-ACN]) to give the compound N-[(1R)-1-[2-(3,4-dihydroxy phenyl)ethylcarbamoyl]-3-(hydroxyamino)-3-oxo-propyl]octanamide (20 mg, 47.38 umol, 3.88% yield, 97% purity) as white solid.

### Example 253: (R,E)-2-(3-cyclohexylacrylamido)-N⁴-hydroxy-N′-(4-hydroxyphenethyl)succinamide

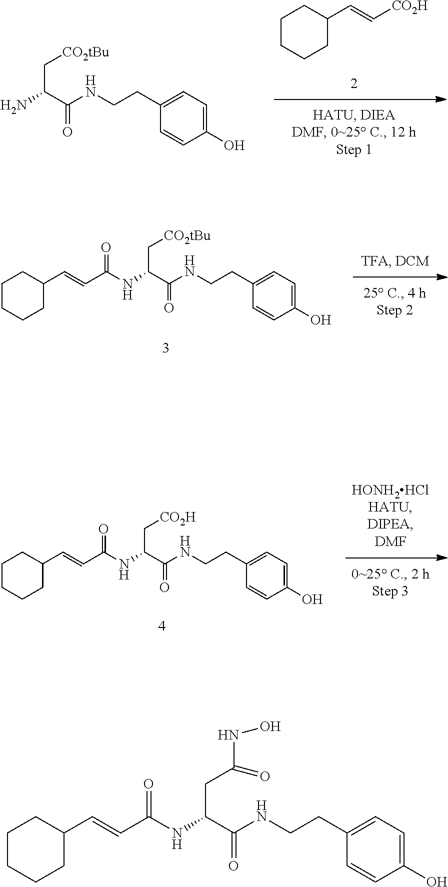 [SMILES: CC(C)(C)OC(=O)C[C@@H](N)C(=O)NCCc1ccc(O)cc1 ; O=C(O)/C=C/C1CCCCC1 ; CC(C)(C)OC(=O)C[C@@H](NC(=O)/C=C/C1CCCCC1)C(=O)NCCc1ccc(O)cc1 ; O=C(O)C[C@@H](NC(=O)/C=C/C1CCCCC1)C(=O)NCCc1ccc(O)cc1 ; O=C(/C=C/C1CCCCC1)N[C@H](CC(=O)NO)C(=O)NCCc1ccc(O)cc1]

### Step 1

To a solution of tert-butyl (3R)-3-amino-4-[2-(4-hydroxyphenyl)ethylamino]-4-oxo-butanoate (960 mg, 3.11 mmol, 1.2 eq) and (E)-3-cyclohexylprop-2-enoic acid (400.05 mg, 2.59 mmol, 1 eq) in DMF (10 mL) was added HATU (1.18 g, 3.11 mmol, 1.2 eq) and DIPEA (737.64 mg, 5.71 mmol, 994.13 uL, 2.2 eq) at 0° C. The mixture was stirred at 25° C. for 12 hr. The mixture was poured into H₂O (30 mL), then extracted with EtOAc (15 mL*3). The combined organic layer was washed with brine (10 mL*2), dried over Na₂SO₄, filtered and the filtrate was concentrated under reduced pressure to give a residue. The residue was purified by column chromatography (SiO₂, Petroleum ether/Ethyl acetate=1/0 to 0/1) to get tert-butyl (3R)-3-[[(E)-3-cyclohexylprop-2-enoyl]amino]-4-[2-(4-hydroxyphenyl)ethylamino]-4-oxo-butanoate (1 g, 1.66 mmol, 64.16% yield, 74% purity) as a yellow solid.

### Step 2

A mixture of tert-butyl (3R)-3-[[(E)-3-cyclohexylprop-2-enoyl]amino]-4-[2-(4-hydroxyphenyl) ethylamino]-4-oxo-butanoate (0.8 g, 1.80 mmol, 1 eq) in DCM (10 mL) and TFA (10 mL) was stirred at 25° C. for 4 hr under N₂ atmosphere. The reaction mixture was concentrated under reduced pressure to get (3R)-3-[[(E)-3-cyclohexylprop-2-enoyl]amino]-4-[2-(4-hydroxyphenyl) ethylamino]-4-oxo-butanoic acid (0.6 g, crude) as a yellow oil.

### Step 3

To a solution of (3R)-3-[[(E)-3-cyclohexylprop-2-enoyl]amino]-4-[2-(4-hydroxyphenyl)ethyl amino]-4-oxo-butanoic acid (0.6 g, 1.54 mmol, 1 eq), HATU (704.75 mg, 1.85 mmol, 1.2 eq) and DIPEA (399.25 mg, 3.09 mmol, 538.07 uL, 2 eq) in DMF (5 mL) was added DIPEA (399.25 mg, 3.09 mmol, 538.07 uL, 2 eq) and hydroxylamine; hydrochloride (214.67 mg, 3.09 mmol, 2 eq) in DMF (5 mL) at 0° C. The mixture was stirred at 25° C. for 3 hr. The reaction mixture was concentrated under reduced pressure to give a residue. The residue was purified by prep-HPLC (column: Phenomenex luna C18 80*40 mm*3 um; mobile phase: [water(0.04% HCl)-ACN]; B %: 35%-45%, 5 min) to get (2R)-2-[[(E)-3-cyclohexylprop-2-enoyl]amino]-4-(hydroxyamino)-N-[2-(4-hydroxyphenyl)ethyl]-4-oxo-butanamide (38 mg, 90.42 umol, 5.85% yield, 96% purity) as a white solid.

### Example 254: (R)-2-(3-cyclohexylpropanamido)-N⁴-hydroxy-N′-(4-hydroxyphenethyl)succinamide

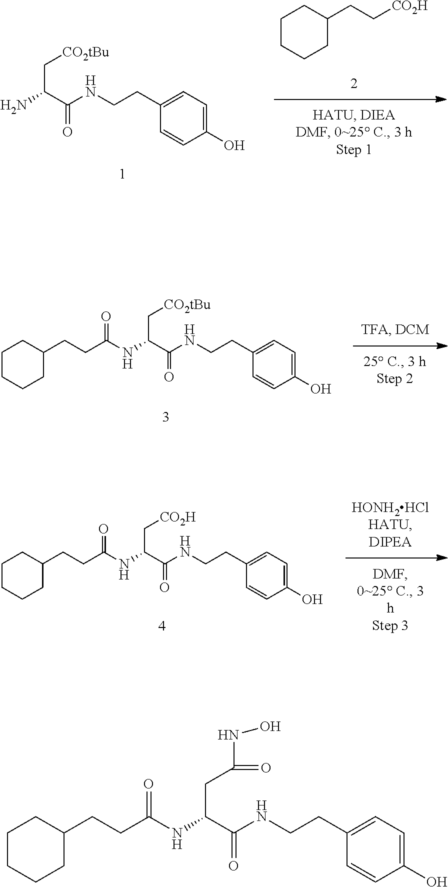 [SMILES: CC(C)(C)OC(=O)C[C@@H](N)C(=O)NCCc1ccc(O)cc1 ; O=C(O)CCC1CCCCC1 ; CC(C)(C)OC(=O)C[C@@H](NC(=O)CCC1CCCCC1)C(=O)NCCc1ccc(O)cc1 ; O=C(O)C[C@@H](NC(=O)CCC1CCCCC1)C(=O)NCCc1ccc(O)cc1 ; O=C(C[C@@H](NC(=O)CCC1CCCCC1)C(=O)NCCc1ccc(O)cc1)NO]

### Step 1

To a solution of 3-cyclohexylpropanoic acid (400 mg, 2.56 mmol, 438.60 uL, 1 eq) and tert-butyl (3R)-3-amino-4-[2-(4-hydroxyphenyl)ethylamino]-4-oxo-butanoate (700 mg, 2.27 mmol, 8.87e-1 eq) in DMF (10 mL) was added HATU (1.17 g, 3.07 mmol, 1.2 eq) and DIEA (728.03 mg, 5.63 mmol, 981.17 uL, 2.2 eq) at 0° C. The mixture was stirred at 25° C. for 3 hr. The reaction mixture was concentrated under reduced pressure to give a residue. The residue was purified by column chromatography (SiO₂, Petroleum ether/Ethyl acetate=15/1 to Ethyl acetate/Petroleum ether=2/1) to give the compound tert-butyl (3R)-3-(3-cyclohexylpropanoylamino)-4-[2-(4-hydroxyphenyl)ethylamino]-4-oxo-butanoate (1 g, 1.99 mmol, 77.83% yield, 89% purity) as white solid.

### Step 2

To a solution of tert-butyl (3R)-3-(3-cyclohexylpropanoylamino)-4-[2-(4-hydroxyphenyl) ethylamino]-4-oxo-butanoate (400 mg, 895.70 umol, 1 eq) in DCM (3 mL) was added TFA (4.62 g, 40.52 mmol, 3 mL, 45.24 eq). The mixture was stirred at 25° C. for 3 hr. The mixture was concentrated under reduced pressure to give the crude product (3R)-3-(3-cyclohexylpro panoylamino)-4-[2-(4-hydroxyphenyl)ethylamino]-4-oxo-butanoic acid (400 mg, crude) as red oil.

### Step 3

To a solution of (3R)-3-(3-cyclohexylpropanoylamino)-4-[2-(4-hydroxyphenyl)ethylamino]-4-oxo-butanoic acid (350 mg, 896.35 umol, 1 eq) and hydroxylamine; hydrochloride (124.58 mg, 1.79 mmol, 2 eq) in DMF (10 mL) was added HATU (374.90 mg, 985.99 umol, 1.1 eq) and DIEA (231.69 mg, 1.79 mmol, 312.26 uL, 2 eq) at 0° C. The mixture was stirred at 25° C. for 3 hr. The reaction mixture was concentrated under reduced pressure to give a residue. The residue was purified by prep-HPLC ([water (0.05% HCl)-ACN]) to give the compound (2R)-2-(3-cyclohexylpropanoylamino)-4-(hydroxyamino)-N-[2-(4-hydroxyphenyl)ethyl]-4-oxo-butanamide (72 mg, 177.56 umol, 19.81% yield) as white solid.

### Example 255: (R)-2-(4-cyclohexylbutanamido)-N4-hydroxy-N′-(4-hydroxyphenethyl)succinamide

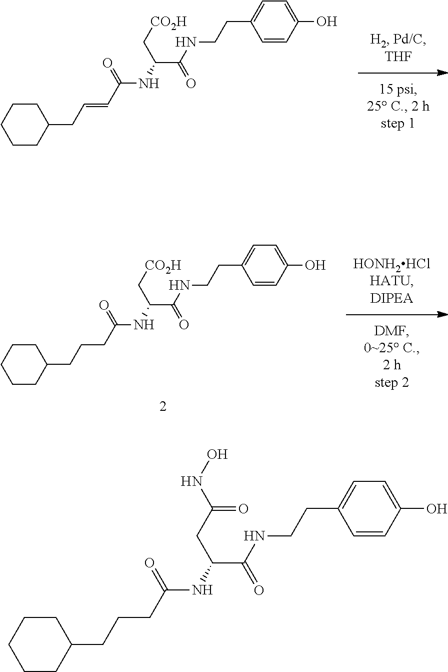 [SMILES: O=C(O)C[C@@H](NC(=O)/C=C/CC1CCCCC1)C(=O)NCCc1ccc(O)cc1 ; O=C(O)C[C@@H](NC(=O)CCCC1CCCCC1)C(=O)NCCc1ccc(O)cc1 ; O=C(C[C@@H](NC(=O)CCCC1CCCCC1)C(=O)NCCc1ccc(O)cc1)NO]

### Step 1

A mixture of (3R)-3-[[(E)-4-cyclohexylbut-2-enoyl]amino]-4-[2-(4-hydroxyphenyl)ethylamino]-4-oxo-butanoic acid (0.5 g, 1.24 mmol, 1 eq) and 10% Pd/C (0.25 g, 1.24 mmol, 1.00 eq) in THF (20 mL) was stirred at 25° C. for 2 hr under H₂ (15 Psi) atmosphere. The reaction mixture was filtered and the filtrate was concentrated under reduced pressure to give the compound (3R)-3-(4-cyclohexylbutanoylamino)-4-[2-(4-hydroxyphenyl) ethylamino]-4-oxo-butanoic acid (0.45 g, 1.11 mmol, 89.55% yield) as a yellow solid.

### Step 2

To a solution of (3R)-3-(4-cyclohexylbutanoylamino)-4-[2-(4-hydroxyphenyl)ethylamino]-4-oxo-butanoic acid (0.4 g, 988.88 umol, 1 eq) in DMF (5 mL) was added DIEA (255.61 mg, 1.98 mmol, 344.48 uL, 2 eq) and HATU (451.20 mg, 1.19 mmol, 1.2 eq) at 0° C., then hydroxylamine; hydrochloride (137.44 mg, 1.98 mmol, 2 eq) and DIEA (255.61 mg, 1.98 mmol, 344.48 uL, 2 eq) in DMF (3 mL) was added to the mixture at 0° C. and stirred at 25° C. for 2 hr. The reaction mixture was concentrated under reduced pressure to give a residue. The residue was purified by p-HPLC (column: Phenomenex luna C18 80*40 mm*3 um; mobile phase: [water(0.04% HCl)-ACN]; B %: 40%-46%, 5 min) to give (2R)-2-(4-cyclohexylbutanoylamino)-4-(hydroxyamino)-N-[2-(4-hydroxyphenyl)ethyl]-4-oxo-butanamide (64 mg, 152.56 umol, 15.43% yield, 100% purity) as a white solid.

### Example 256: (R,E)-2-(4-cyclohexylbut-2-enamido)-N⁴-hydroxy-N′-(4-hydroxyphenethyl)succinamide

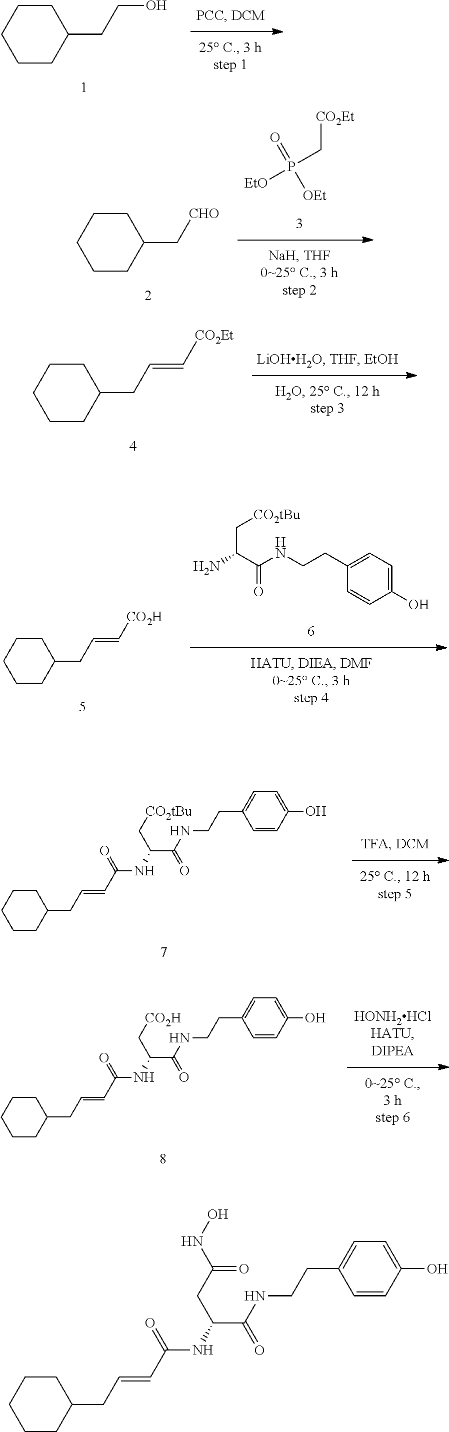

### Step 1

To a solution of 2-cyclohexylethanol (4 g, 31.20 mmol, 4.35 mL, 1 eq) in DCM (1000 mL) was added PCC (10.09 g, 46.80 mmol, 1.5 eq). The mixture was stirred at 25° C. for 3 hr. The reaction mixture was diluted with MTBE (800 mL) and stirred at 25° C. for 1 h, before it was filtered through a pad of celite and silica gel (10 g, 1:1) and the filtrate was carefully concentrated in vacuum at 30° C. to give the compound 2-cyclohexylacetaldehyde (3.9 g, crude) as a yellow oil.

### Step 2

To a solution of NaH (1.61 g, 40.18 mmol, 60%, 1.3 eq) in THF (80 mL) was added ethyl 2-diethoxyphosphorylacetate (7.27 g, 32.45 mmol, 6.44 mL, 1.05 eq) at 0° C., then 2-cyclohexylacetaldehyde (3.9 g, 30.90 mmol, 1 eq) in THF (20 mL) was added dropwise to the mixture at 0° C. and stirred at 25° C. for 2.5 hr. The mixture was quenched by sat. NH₄Cl (50 mL) aq. and the mixture was extracted with ethyl acetate (50 mL*2). The combined organic phase was washed with brine (50 mL), dried with anhydrous Na₂SO₄, filtered and the filtrate was concentrated in vacuum to give the compound ethyl (E)-4-cyclohexylbut-2-enoate (6 g, crude) as a yellow oil.

### Step 3

To a solution of ethyl (E)-4-cyclohexylbut-2-enoate (6 g, 30.57 mmol, 1 eq) in THF (20 mL), EtOH (20 mL) and H₂O (20 mL) was added LiOH·H₂O (6.41 g, 152.84 mmol, 5 eq). The mixture was stirred at 25° C. for 12 hr. To the mixture was added H₂O (50 mL) and the mixture was extracted with ethyl acetate (50 mL). The aqueous phase was adjusted pH=2-3 with 4N HCl. and extracted with ethyl acetate (30 mL*4). The combined organic phase was washed with brine (50 mL), dried with anhydrous Na₂SO₄, filtered and the filtrate was concentrated in vacuum to give (E)-4-cyclohexylbut-2-enoic acid (4 g, 23.78 mmol, 77.78% yield) as a yellow oil.

### Step 4

To a solution of (E)-4-cyclohexylbut-2-enoic acid (1 g, 5.94 mmol, 1 eq) and tert-butyl (3R)-3-amino-4-[2-(4-hydroxyphenyl)ethylamino]-4-oxo-butanoate (2.02 g, 6.54 mmol, 1.1 eq) in DMF (20 mL) was added HATU (2.71 g, 7.13 mmol, 1.2 eq) and DIEA (1.69 g, 13.08 mmol, 2.28 mL, 2.2 eq) at 0° C. The mixture was stirred at 25° C. for 3 hr. The reaction mixture was quenched by addition H₂O 20 mL at 0° C., and then diluted with EtOAc 20 mL and extracted with EtOAc 60 mL (20 mL*3). The combined organic layers were washed with brine 30 mL, dried over Na₂SO₄, filtered and the filtrate was concentrated under reduced pressure to give a residue. The residue was purified by column chromatography (SiO₂, Petroleum ether/Ethyl acetate=1/0 to 0/1) to give the compound tert-butyl (3R)-3-[[(E)-4-cyclohexylbut-2-enoyl]amino]-4-[2-(4-hydroxyphenyl) ethylamino]-4-oxo-butanoate (1.1 g, 2.40 mmol, 40.35% yield) as a yellow solid.

### Step 5

A mixture of tert-butyl (3R)-3-[[(E)-4-cyclohexylbut-2-enoyl]amino]-4-[2-(4-hydroxyphenyl) ethylamino]-4-oxo-butanoate (1.1 g, 2.40 mmol, 1 eq) in DCM (10 mL) and TFA (10 mL) was stirred at 25° C. for 12 hr under N₂ atmosphere. The reaction mixture was concentrated under reduced pressure to give the compound (3R)-3-[[(E)-4-cyclohexylbut-2-enoyl]amino]-4-[2-(4-hydroxyphenyl)ethylamino]-4-oxo-butanoic acid (1 g, crude) as a yellow solid.

### Step 6

To a solution of (3R)-3-[[(E)-4-cyclohexylbut-2-enoyl]amino]-4-[2-(4-hydroxyphenyl) ethylamino]-4-oxo-butanoic acid (0.45 g, 1.12 mmol, 1 eq) in DMF (3 mL) was added HATU (510.14 mg, 1.34 mmol, 1.2 eq) and DIEA (289.00 mg, 2.24 mmol, 389.49 uL, 2 eq) at 0° C. The hydroxylamine hydrochloride (155.39 mg, 2.24 mmol, 2 eq) and DIEA (289.00 mg, 2.24 mmol, 389.49 uL, 2 eq) in DMF (2 mL) was added to the mixture at 0° C. and stirred at 25° C. for 3 hr. The reaction mixture was concentrated under reduced pressure to give a residue. The residue was purified by prep-HPLC (column: Phenomenex luna C18 80*40 mm*3 um; mobile phase: [water(0.04% HCl)-ACN]; B %: 40%-46%, 5 min) to give (2R)-2-[[(E)-4-cyclohexylbut-2-enoyl]amino]-4-(hydroxyamino)-N-[2-(4-hydroxyphenyl)ethyl]-4-oxo-butanamide (86 mg, 201.87 umol, 18.06% yield, 98% purity) as a yellow solid.

### Example 257: (R,E)-2-(4-cyclopentylbut-2-enamido)-N⁴-hydroxy-N′-(4-hydroxyphenethyl)succinimide

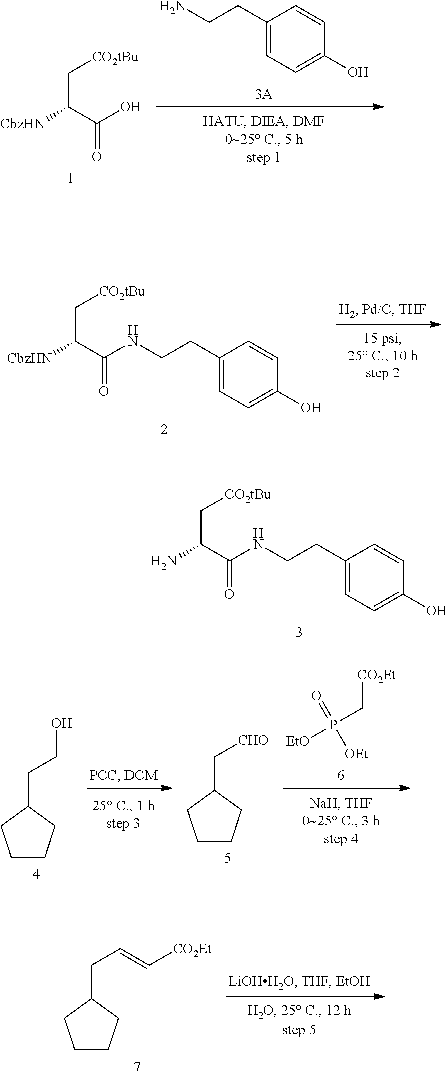

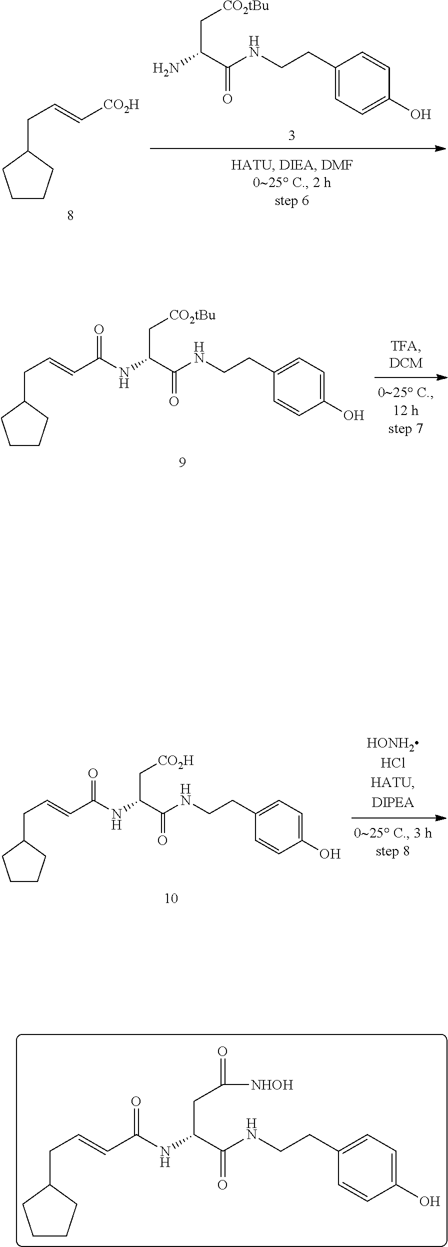

### Step 1

A mixture of (2R)-2-(benzyloxycarbonylamino)-4-tert-butoxy-4-oxo-butanoic acid; hydrate (15 g, 43.95 mmol, 1 eq), 4-(2-aminoethyl)phenol (7.23 g, 52.74 mmol, 1.2 eq), HATU (20.04 g, 52.74 mmol, 1.2 eq), in DMF (200 mL) was added DIEA (12.48 g, 96.66 mmol, 16.83 mL, 2.2 eq) at 0° C. The mixture was stirred at 25° C. for 5 hr under N₂ atmosphere. The reaction mixture was quenched by addition H₂O 500 mL at 0° C., and extracted with EtOAc 900 mL (300 mL*3). The combined organic layers were washed with brine 150 mL, dried over Na₂SO₄, filtered and concentrated under reduced pressure to give a residue. The residue was purified by column chromatography (SiO₂, Petroleum ether/Ethyl acetate=10/1 to 0/1) to give the compound tert-butyl (3R)-3-(benzyloxycarbonylamino)-4-[2-(4-hydroxyphenyl)ethylamino]-4-oxo-butanoate (12 g, 26.58 mmol, 60.48% yield, 98% purity) as a yellow oil.

### Step 2

A mixture of tert-butyl (3R)-3-(benzyloxycarbonylamino)-4-[2-(4-hydroxyphenyl)ethylamino]-4-oxo-butanoate (12 g, 27.12 mmol, 1 eq), and 10% Pd/C (2 g,) in THF (500 mL) was stirred at 25° C. for 10 hr under H₂ atmosphere (15 psi). The reaction mixture was filtered and the filtrate was concentrated under reduced pressure to give the compound tert-butyl (3R)-3-amino-4-[2-(4-hydroxyphenyl)ethylamino]-4-oxo-butanoate (7 g, 22.70 mmol, 83.71% yield) as a yellow oil.

### Step 3

To a solution of 2-cyclopentylethanol (5 g, 43.79 mmol, 5.43 mL, 1 eq) in DCM (1500 mL) was added PCC (14.16 g, 65.68 mmol, 1.5 eq). The mixture was stirred at 25° C. for 1 hr. The reaction mixture was diluted with MTBE (800 mL) stirred at 25° C. for 1 h, and filtered through a pad of celite and silica gel (1:1). The filtrate was carefully concentrated to give the compound 2-cyclopentylacetaldehyde (5 g, crude) as a brown solid.

### Step 4

To a solution of NaH (2.32 g, 57.95 mmol, 60% purity, 1.3 eq) in THF (175 mL) was added ethyl 2-diethoxyphosphorylacetate (9.99 g, 44.58 mmol, 8.84 mL, 1 eq) at 0° C. and stirred for 5 min. Then 2-cyclopentylacetaldehyde (5 g, 44.58 mmol, 1 eq) was added to the mixture at 0° C. and stirred at 0° C. for 25 min. The mixture was stirred at 25° C. for 1.5 h. The mixture was quenched by sat. NH₄Cl (100 mL) and the mixture was extracted with ethyl acetate (100 mL*2). The combined organic phase was washed with brine (100 mL), dried with anhydrous Na₂SO₄, filtered and concentrated in vacuum to give a residue. The residue was purified by column chromatography (SiO₂, Petroleum ether/Ethyl acetate=1/0 to 10/1) to give the compound ethyl (E)-4-cyclopentylbut-2-enoate (5 g, 27.43 mmol, 61.54% yield) as yellow oil.

### Step 5

To a solution of ethyl (E)-4-cyclopentylbut-2-enoate (5 g, 27.43 mmol, 1 eq) in THF (20 mL), EtOH (20 mL), H₂O (20 mL) was added LiOH·H₂O (5.76 g, 137.17 mmol, 5 eq). The mixture was stirred at 25° C. for 12 hr. To the mixture was added water (50 mL) and the mixture was extracted with ethyl acetate (50 mL). Then the aqueous phase treated with 4 N HCl to pH=2-3. The aqueous phase was extracted with ethyl acetate (50 mL*4). The combined organic phase was dried with anhydrous Na₂SO₄, filtered and concentrated in vacuum to give the compound (E)-4-cyclopentylbut-2-enoic acid (3.8 g, 24.64 mmol, 89.83% yield) as yellow liquid.

### Step 6

To a solution of tert-butyl (3R)-3-amino-4-[2-(4-hydroxyphenyl)ethylamino]-4-oxo-butanoate (1.5 g, 4.86 mmol, 1 eq) and (E)-4-cyclopentylbut-2-enoic acid (900.12 mg, 5.84 mmol, 1.2 eq) in DMF (15 mL) was added HATU (2.22 g, 5.84 mmol, 1.2 eq) and DIPEA (1.38 g, 10.70 mmol, 1.86 mL, 2.2 eq) at 0° C. The mixture was stirred at 25° C. for 2 hr. The mixture was concentrated under reduced pressure to give a residue. The residue was purified by column chromatography (SiO₂, Petroleum ether/Ethyl acetate=10/1 to 1/1) to give the compound tert-butyl (3R)-3-[[(E)-4-cyclopentylbut-2-enoyl]amino]-4-[2-(4-hydroxyphenyl)ethylamino]-4-oxo-butanoate (2 g, 4.50 mmol, 92.49% yield) as brown oil.

### Step 7

To a solution of tert-butyl (3R)-3-[[(E)-4-cyclopentylbut-2-enoyl]amino]-4-[2-(4-hydroxyphenyl) ethylamino]-4-oxo-butanoate (2 g, 4.50 mmol, 1 eq) in DCM (10 mL) was added TFA (10 mL) at 0° C. The mixture was stirred at 25° C. for 12 hr. The mixture was concentrated under reduced pressure to give the compound (3R)-3-[[(E)-4-cyclopentylbut-2-enoyl]amino]-4-[2-(4-hydroxyphenyl)ethylamino]-4-oxo-butanoic acid (1.5 g, crude) as brown oil.

### Step 8

To a solution of (3R)-3-[[(E)-4-cyclopentylbut-2-enoyl]amino]-4-[2-(4-hydroxyphenyl)ethylamino]-4-oxo-butanoic acid (500 mg, 1.29 mmol, 1 eq) in DMF (5 mL) was added HATU (538.35 mg, 1.42 mmol, 1.1 eq) and DIPEA (332.71 mg, 2.57 mmol, 448.40 uL, 2 eq), then hydroxylamine hydrochloride (178.89 mg, 2.57 mmol, 2 eq) in DMF (1.2 mL) and DIPEA (332.71 mg, 2.57 mmol, 448.40 uL, 2 eq) was added to the mixture at 0° C. The mixture was stirred at 25° C. for 3 hr. The mixture was concentrated under reduced pressure to give a residue. The residue was purified by prep-HPLC (column: Welch Xtimate C18 100*25 mm*3 um; mobile phase: [water(0.05% HCl)-ACN]; B %: 20%-50%, 8 min) to give the compound (2R)-2-[[(E)-4-cyclopentylbut-2-enoyl]amino]-4-(hydroxyamino)-N-[2-(4-hydroxyphenyl)ethyl]-4-oxo-butanamide (82 mg, 197.14 umol, 15.32% yield, 97% purity) as a white solid.

### Example 258: (R)-2-(4-cyclopentylbutanamido)-N⁴-hydroxy-N′-(4-hydroxyphenethyl)succinamide

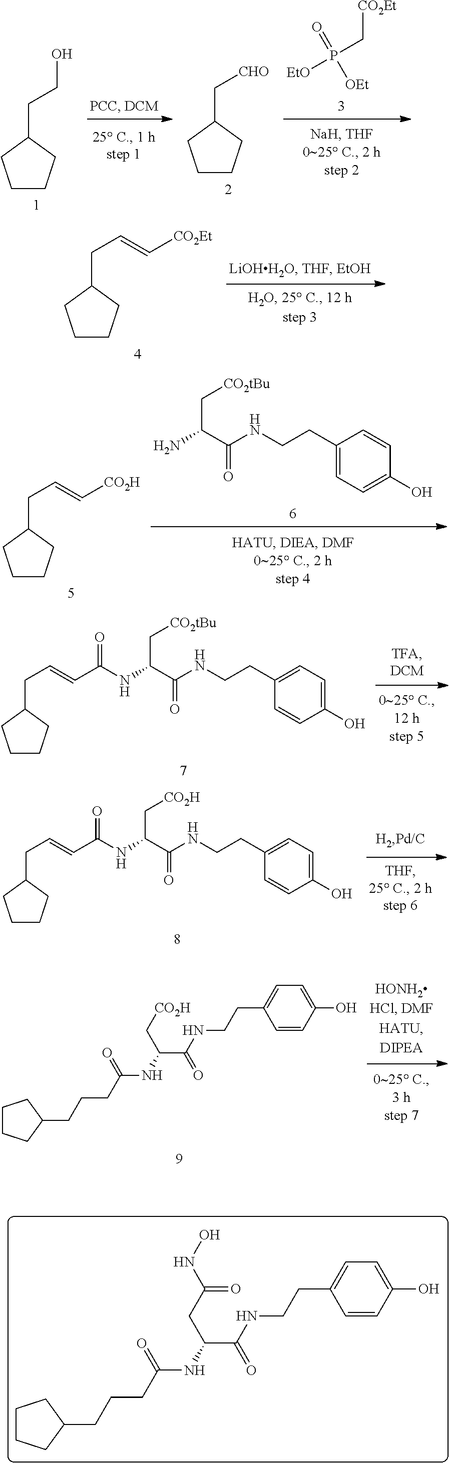

### Step 1

To a solution of 2-cyclopentylethanol (5 g, 43.79 mmol, 5.43 mL, 1 eq) in DCM (1500 mL) was added PCC (14.16 g, 65.68 mmol, 1.5 eq). The mixture was stirred at 25° C. for 1 hr. The reaction mixture was diluted with MTBE (800 mL) stirred at 25° C. for 1 h, then filtered through a pad of celite and silica gel (1:1). The filtrate was carefully concentrated in vacuum to get the compound 2-cyclopentylacetaldehyde (5 g, crude) as a brown solid.

### Step 2

To a solution of NaH (2.32 g, 57.95 mmol, 60%, 1.3 eq) in THF (175 mL) was added ethyl 2-diethoxyphosphorylacetate (9.99 g, 44.58 mmol, 8.84 mL, 1 eq) at 0° C. and stirred for 5 min. Then 2-cyclopentylacetaldehyde (5 g, 44.58 mmol, 1 eq) was added to the mixture at 0° C. for 25 min. The mixture was stirred at 25° C. for 1.5 h. TLC showed one new spot was formed. The mixture was quenched by sat·NH₄Cl aq. (100 mL) and the mixture was extracted with ethyl acetate (100 mL*2). The combined organic phase was washed with brine (100 mL), dried with anhydrous Na₂SO₄, filtered and the filtrate was concentrated in vacuum to get a residue. The residue was purified by column chromatography (SiO₂, Petroleum ether/Ethyl acetate=1/0 to 10/1) to give the compound ethyl (E)-4-cyclopentylbut-2-enoate (5 g, 27.43 mmol, 61.54% yield) as yellow oil.

### Step 3

To a solution of ethyl (E)-4-cyclopentylbut-2-enoate (5 g, 27.43 mmol, 1 eq) in THF (20 mL), EtOH (20 mL), H₂O (20 mL) was added LiOH·H₂O (5.76 g, 137.17 mmol, 5 eq). The mixture was stirred at 25° C. for 12 hr. LCMS showed 78% of the desired product. H₂O (50 mL) was added to the mixture and the mixture was extracted with ethyl acetate (50 mL). Then the aqueous phase treated with 4 N HCl to pH=2-3. The aqueous phase was extracted with ethyl acetate (50 mL*4). The combined organic phase was dried with anhydrous Na₂SO₄, filtered and concentrated in vacuum to the compound (E)-4-cyclopentylbut-2-enoic acid (3.8 g, 24.64 mmol, 89.83% yield) as yellow liquid.

### Step 4

To a solution of tert-butyl (3R)-3-amino-4-[2-(4-hydroxyphenyl)ethylamino]-4-oxo-butanoate (1.5 g, 4.86 mmol, 1 eq) and (E)-4-cyclopentylbut-2-enoic acid (900.12 mg, 5.84 mmol, 1.2 eq) in DMF (15 mL) was added HATU (2.22 g, 5.84 mmol, 1.2 eq) and DIPEA (1.38 g, 10.70 mmol, 1.86 mL, 2.2 eq) at 0° C. The mixture was stirred at 25° C. for 2 hr. TLC showed the starting reactant was consumed and two new spots were formed. The mixture was concentrated under reduced pressure to give a residue. The residue was purified by column chromatography (SiO₂, Petroleum ether/Ethyl acetate=10/1 to 1/1) to give the compound tert-butyl (3R)-3-[[(E)-4-cyclopentylbut-2-enoyl]amino]-4-[2-(4-hydroxyphenyl)ethylamino]-4-oxo-butanoate (2 g, 4.50 mmol, 92.49% yield) as brown oil.

### Step 5

To a solution of tert-butyl (3R)-3-[[(E)-4-cyclopentylbut-2-enoyl]amino]-4-[2-(4-hydroxyphenyl) ethylamino]-4-oxo-butanoate (2 g, 4.50 mmol, 1 eq) in DCM (10 mL) was added TFA (10 mL) at 0° C. The mixture was stirred at 25° C. for 12 hr. The mixture was concentrated under reduced pressure to give the compound (3R)-3-[[(E)-4-cyclopentylbut-2-enoyl]amino]-4-[2-(4-hydroxy phenyl)ethylamino]-4-oxo-butanoic acid (1.5 g, crude) as brown oil.

### Step 6

To a solution of (3R)-3-[[(E)-4-cyclopentylbut-2-enoyl]amino]-4-[2-(4-hydroxyphenyl)ethyl amino]-4-oxo-butanoic acid (500 mg, 1.29 mmol, 1 eq) in THF (5 mL) was added 10% Pd/C (0.2 g, 1.00 eq). The mixture was stirred at 25° C. for 2 hr under H₂ (15 psi). The mixture was filtered and the filtrate was concentrated under reduced pressure to give the compound (3R)-3-(4-cyclopentylbutanoylamino)-4-[2-(4-hydroxyphenyl) ethylamino]-4-oxo-butanoic acid (360 mg, 921.96 umol, 71.63% yield) as a white solid.

### Step 7

To a mixture of (3R)-3-(4-cyclopentylbutanoylamino)-4-[2-(4-hydroxyphenyl)ethylamino]-4-oxo-butanoic acid (300 mg, 768.30 umol, 1 eq), HATU (321.34 mg, 845.13 umol, 1.1 eq), DIPEA (198.59 mg, 1.54 mmol, 267.65 uL, 2 eq) in DMF (5 mL) was added a mixture of hydroxylamine hydrochloride (106.78 mg, 1.54 mmol, 2 eq), DIPEA (198.59 mg, 1.54 mmol, 267.65 uL, 2 eq) and DMF (1 mL) in portions at 0° C. The resulting mixture was stirred at 25° C. for 3 hr. The mixture was concentrated under reduced pressure to give a residue. The residue was purified by prep-HPLC (column: Welch Xtimate C18 100*25 mm*3 um; mobile phase: [water(0.05% HCl)-ACN]; B %: 20%-50%, 8 min) to give the compound (2R)-2-(4-cyclopentylbutanoylamino)-4-(hydroxyamino)-N-[2-(4-hydroxyphenyl)ethyl]-4-oxo-butanamide (114 mg, 281.14 umol, 36.59% yield, 100% purity) as a white solid.

### Example 259: (R)-N′-(4-hydroxyphenethyl)-2-(6-(methylamino)hexanamido)succinamide

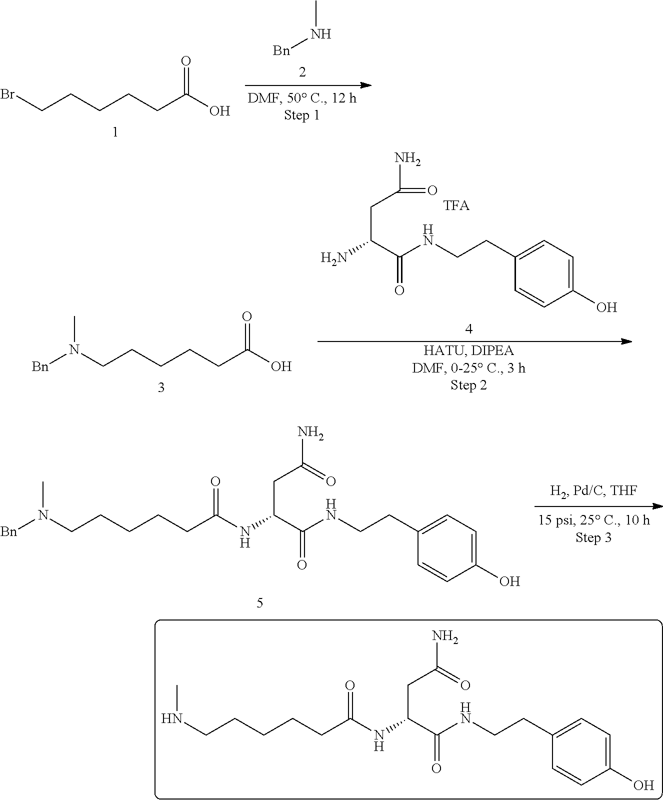

### Step 1

To a solution of 6-bromohexanoic acid (1 g, 5.13 mmol, 1 eq) in DMF (10 mL) was added N-methyl-1-phenyl-methanamine (3.11 g, 25.63 mmol, 3.31 mL, 5 eq). The mixture was stirred at 50° C. for 12 hr. The reaction mixture was concentrated under reduced pressure to give a residue. The residue was purified by column chromatography (SiO₂, Petroleum ether/Ethyl acetate=1/1 to Ethyl acetate/MeOH=0/1) to give the compound 6-[benzyl(methyl)amino]hexanoic acid (800 mg, 3.40 mmol, 66.31% yield) as yellow oil.

### Step 2

To a solution of 6-[benzyl(methyl)amino]hexanoic acid (100 mg, 424.95 umol, 1 eq) and (2R)-2-amino-N-[2-(4-hydroxyphenyl)ethyl]butanediamide (201.81 mg, 552.44 umol, 1.3 eq, TFA) in DMF (5 mL) was added HATU (193.89 mg, 509.94 umol, 1.2 eq) and DIEA (137.30 mg, 1.06 mmol, 185.05 uL, 2.5 eq) at 0° C. The mixture was stirred at 25° C. for 3 hr. The reaction mixture was concentrated under reduced pressure to give a residue. The residue was purified by column chromatography (SiO₂, Petroleum ether/Ethyl acetate=0/1 to Ethyl acetate/MeOH=0/1) and prep-HPLC ([water (10 mM NH₄HCO₃)-ACN]) to give the compound (2R)-2-[6-[benzyl(methyl)amino]hexanoylamino]-N-[2-(4-hydroxyphenyl) ethyl]butanediamide (12 mg, 25.61 umol, 6.03% yield) as white solid.

### Step 3

To a solution of (2R)-2-[6-[benzyl(methyl)amino]hexanoylamino]-N-[2-(4-hydroxyphenyl) ethyl]butanediamide (80 mg, 170.73 umol, 1 eq) in MeOH (10 mL) was added Pd/C (100 mg, 10% purity) and stirred under H₂ (15 Psi) at 25° C. for 10 hr. LCMS showed the desired compound was detected. The reaction mixture was concentrated under reduced pressure to give a residue. The residue was purified by prep-HPLC ([water(10 mM NH₄HCO₃)-ACN]) to give the compound (2R)-N-[2-(4-hydroxyphenyl)ethyl]-2-[6-(methylamino)hexanoylamino]butanediamide (8 mg, 21.14 umol, 12.38% yield) as white solid.

### Example 260: (R)-2-octanamido-N′-((R)-2-phenylpropyl)succinamide

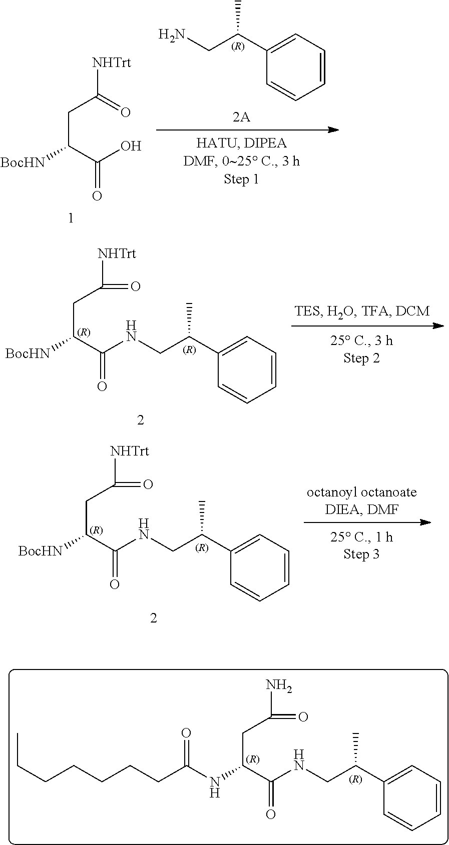

### Step 1

To a solution of (2R)-2-(tert-butoxycarbonylamino)-4-oxo-4-(tritylamino)butanoic acid (1 g, 2.11 mmol, 1 eq) and (2R)-2-phenylpropan-1-amine (341.90 mg, 2.53 mmol, 361.80 uL, 1.2 eq) in DMF (10 mL) was added HATU (961.50 mg, 2.53 mmol, 1.2 eq) and DIEA (599.17 mg, 4.64 mmol, 807.51 uL, 2.2 eq) at 0° C. The mixture was stirred at 25° C. for 3 hr. The reaction mixture was poured into H₂O (20 ml), filtered and the filter cake was concentrated under reduced pressure to give the compound tert-butyl N-[(1R)-3-oxo-1-[[(2R)-2-phenylpropyl]carbamoyl]-3-(tritylamino)propyl]carbamate (1 g, 1.62 mmol, 76.99% yield, 96% purity) as white solid.

### Step 2

A mixture of TFA (24.95 g, 218.80 mmol, 16.20 mL, 143.86 eq), DCM (1.19 g, 13.99 mmol, 900.00 uL, 9.20 eq), H₂O (450.00 mg, 24.98 mmol, 450.00 uL, 16.42 eq), triethylsilane (327.60 mg, 2.82 mmol, 450.00 uL, 1.85 eq) and tert-butyl N-[(1R)-3-oxo-1-[[(2R)-2-phenylpropyl]carbamoyl]-3-(tritylamino)propyl]carbamate (900 mg, 1.52 mmol, 1 eq), then the mixture was stirred at 25° C. for 3 hr under N₂ atmosphere. The reaction mixture was concentrated under reduced pressure to give the crude product (2R)-2-amino-N-[(2R)-2-phenylpropyl]butanediamide (500 mg, crude, TFA) as white solid.

### Step 3

To a solution of (2R)-2-amino-N-[(2R)-2-phenylpropyl]butanediamide (500 mg, 1.38 mmol, 1 eq, TFA) in DMF (5 mL) was added octanoyl octanoate (334.91 mg, 1.24 mmol, 0.9 eq) and DIEA (213.43 mg, 1.65 mmol, 287.64 uL, 1.2 eq). The mixture was stirred at 25° C. for 1 hr. The reaction mixture was concentrated under reduced pressure to give the residue. The residue was purified by re-crystallization from DMF:EtOAc=1:1 (10 mL) to give the compound (2R)-2-(octanoylamino)-N-[(2R)-2-phenylpropyl]butanediamide (170 mg, 443.67 umol, 23.80% yield, 98% purity) as white solid.

### Example 261: (R)-2-octanamido-N′-((S)-2-phenylpropyl)succinamide

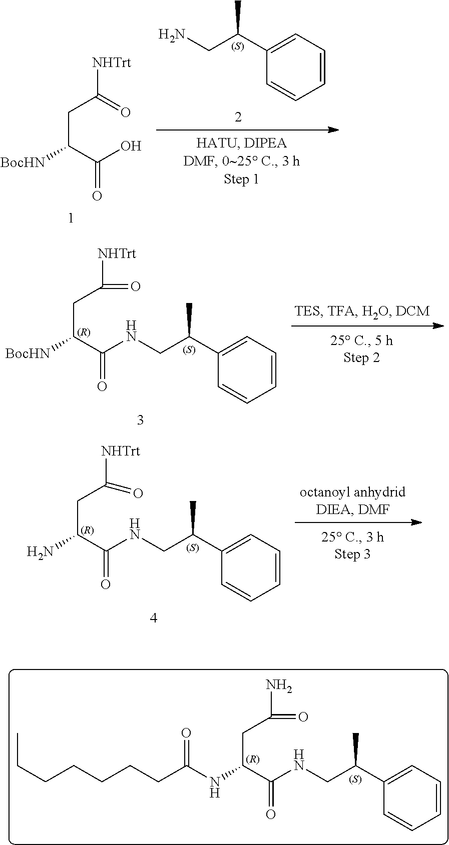

### Step 1

To a solution of (2R)-2-(tert-butoxycarbonylamino)-4-oxo-4-(tritylamino)butanoic acid (2.00 g, 4.21 mmol, 1 eq) and (2S)-2-phenylpropan-1-amine (683.80 mg, 5.06 mmol, 723.60 uL, 1.2 eq) in DMF (20 mL) was added HATU (1.92 g, 5.06 mmol, 1.2 eq) and DIPEA (1.20 g, 9.27 mmol, 1.61 mL, 2.2 eq) at 0° C. The mixture was stirred at 25° C. for 3 hr. The reaction mixture was poured into H₂O (20 mL), filtered and the filter cake was concentrated under reduced pressure to give tert-butylN-[(1R)-3-oxo-1-[[(2S)-2-phenylpropyl]carbamoyl]-3-(tritylamino)propyl]carbamate (1.8 g, 2.92 mmol, 69.29% yield, 96% purity) as a white solid.

### Step 2

A mixture of tert-butyl N-[(1R)-3-oxo-1-[[(2S)-2-phenylpropyl]carbamoyl]-3-(tritylamino) propyl]carbamate (1.3 g, 2.20 mmol, 1 eq), TES (0.25 mL), H₂O (0.25 mL), DCM (0.5 mL) and TFA (9 mL) was stirred at 25° C. for 5 hr under N₂ atmosphere. The reaction mixture was concentrated under reduced pressure to give (2R)-2-amino-N-[(2S)-2-phenylpropyl]butanediamide (0.58 g, crude, TFA) as a yellow solid.

### Step 3

To a solution of (2R)-2-amino-N-[(2S)-2-phenylpropyl]butanediamide (0.58 g, 1.60 mmol, 1 eq, TFA) in DMF (10 mL) was added DIEA (618.95 mg, 4.79 mmol, 834.16 uL, 3 eq) and octanoyl octanoate (388.50 mg, 1.44 mmol, 0.9 eq). The mixture was stirred at 25° C. for 3 hr. The reaction mixture was poured into H₂O (25 mL), filtered and the filter cake was concentrated under reduced pressure to give a residue. The residue was triturated with EtOAc:DMF=1:1 (5 mL) at 25° C. for 5 min, filtered and the filter cake was concentrated under reduced pressure to give (2R)-2-(octanoylamino)-N-[(2S)-2-phenylpropyl]butanediamide (36 mg, 90.12 umol, 5.65% yield, 94% purity) as a white solid.

### Example 262: (R)-2-(4-cyclohexylbutanamido)-N′-(2-(pyridin-2-yl)ethyl)succinamide

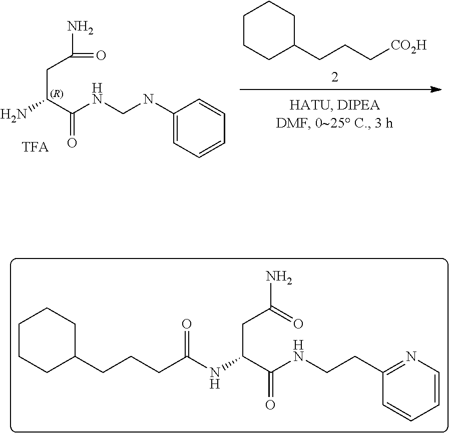

To a mixture of (2R)-2-amino-N-[2-(2-pyridyl)ethyl]butanediamide (400 mg TFA) and 4-cyclohexylbutanoic acid (125.98 mg, 739.95 umol, 1.2 eq) in DMF (12 mL) was added HATU (281.35 mg, 739.95 umol, 1.2 eq) and DIPEA (557.85 mg, 4.32 mmol, 751.82 uL, 7 eq) at 0° C. under N₂. The mixture was stirred at 25° C. for 3 hour. The mixture was added H₂O (20 mL) slowly, then the precipitate was filtered and the filter cake was washed by H₂O (30 mL). The filter cake was concentrated in vacuum to get a residue. The residue was purified by prep-HPLC (column: Phenomenex Gemini-NX 80*40 mm*3 um; mobile phase: [water(10 mM NH4HCO3)-ACN]; B %: 10%-50%, 8 min) to give (2R)-2-(4-cyclohexylbutanoylamino)-N-[2-(2-pyridyl)ethyl]butanediamide (13 mg, 94.70% purity) as a white solid.

### Example 263: (R)-N′-(4-hydroxyphenethyl)-2-(10,10,10-trifluorodecanamido)succinamide

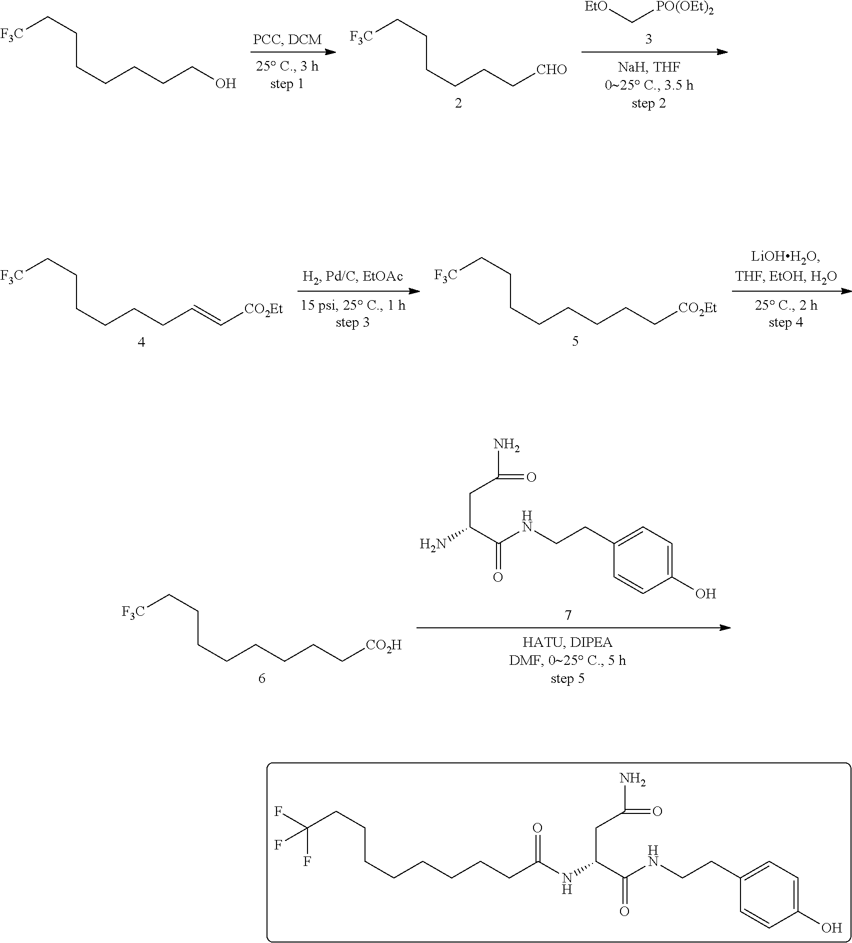

### Step 1

To a solution of 8,8,8-trifluorooctan-1-ol (1 g, 5.43 mmol, 1 eq) in DCM (250 mL) was added PCC (1.76 g, 8.14 mmol, 1.5 eq) and stirred at 25° C. for 3 h. The reaction mixture was diluted with MTBE (150 mL) stirred at 25° C. for 1 h, before it was filtered through a pad of celite and silica gel (1:1). The filtrate was concentrated to give the compound 8,8,8-trifluorooctanal (1 g, crude) as yellow oil.

### Step 2

To a mixture of NaH (285.43 mg, 7.14 mmol, 60% purity, 1.3 eq) in THF (25 mL) was added ethyl 2-diethoxyphosphorylacetate (1.29 g, 5.76 mmol, 1.14 mL, 1.05 eq) dropwise at 0° C. under N2. The mixture was stirred at 0° C. for 5 min. Then a solution of 8,8,8-trifluorooctanal (1 g, 5.49 mmol, 1 eq) in THF (10 mL) was added dropwise at 0° C. The mixture was stirred at 0° C. for 25 min and then warmed to 25° C. stirred for 3 hr. The mixture was quenched by sat·NH₄Cl (50 mL) and the mixture was extracted with ethyl acetate (30 mL*2). The combined organic phase was washed with brine (50 mL), dried with anhydrous Na₂SO₄, filtered and concentrated in vacuum to give the compound ethyl (E)-10,10,10-trifluorodec-2-enoate (1.4 g, crude) as colorless oil.

### Step 3

To a solution of ethyl (E)-10,10,10-trifluorodec-2-enoate (1.4 g, 5.55 mmol, 1 eq) in EtOAc (40 mL) was added 10% Pd/C (0.5 g,) under N₂. The suspension was degassed under vacuum and purged with H₂ three times. The mixture was stirred under H₂ (15 psi) at 25° C. for 1 hour. The mixture was filtered through celite and the filtrate was concentrated to give the compound ethyl 10,10,10-trifluorodecanoate (1.5 g, crude) as yellow oil.

### Step 4

To a mixture of ethyl 10,10,10-trifluorodecanoate (1.5 g, 5.90 mmol, 1 eq) in THF (5 mL), EtOH (5 mL) and H₂O (5 mL) was added LiOH·H₂O (1.24 g, 29.49 mmol, 5 eq) in one portion at 25° C. The mixture was stirred at 25° C. for 2 hours. To the mixture was added water (20 mL) and washed with ethyl acetate (20 mL). Then the aqueous phase was adjusted pH=2-3 with 4N HCl (20 mL). The aqueous phase was extracted with ethyl acetate (20 mL*4). The combined organic phase was washed with brine (50 mL), dried with anhydrous Na₂SO₄, filtered and concentrated in vacuum. The residue was used directly without purification to give the compound 10,10,10-trifluorodecanoic acid (1 g, crude) as colorless oil.

### Step 5

A mixture of 10,10,10-trifluorodecanoic acid (300 mg, 1.33 mmol, 1 eq), (2R)-2-amino-N-[2-(4-hydroxyphenyl)ethyl]butanediamide (1.35 g, 1.59 mmol, 43% purity, 1.2 eq, TFA), HATU (605.05 mg, 1.59 mmol, 1.2 eq) in DMF (3 mL) was added DIEA (377.03 mg, 2.92 mmol, 508.13 uL, 2.2 eq) and was degassed and purged with N2 for 3 times at 0° C. The mixture was stirred at 25° C. for 5 hr under N₂ atmosphere. The reaction mixture was added H₂O 10 mL and was filtered. The filter cake was concentrated under reduced pressure to give a residue. The residue was purified by flash silica gel chromatography (column: Welch Ultimate XB NH₂ 10 u 100*30 mm; mobile phase: [Heptane-EtOH]; B %: 10%-70%, 10 min) to give the compound (2R)-N-[2-(4-hydroxyphenyl)ethyl]-2-(10,10,10-trifluorodecanoylamino)butanediamide (100 mg, 206.75 umol, 15.59% yield, 95% purity) as white solid.

### Example 264: (R)-N′-(4-hydroxyphenethyl)-2-(8,8,8-trifluorooctanamido)succinamide

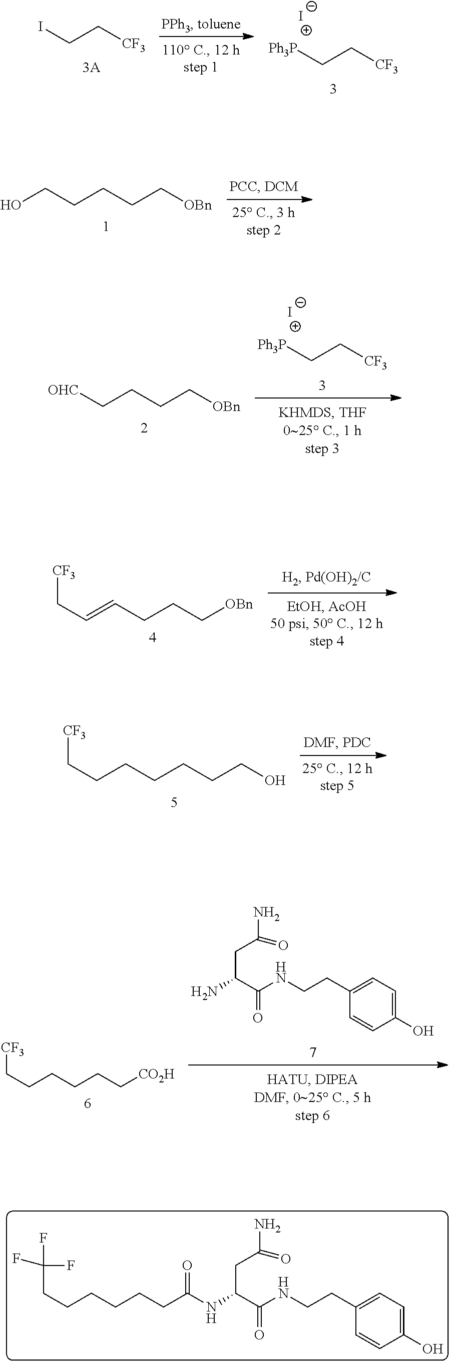

### Step 1

A mixture of 1,1,1-trifluoro-3-iodo-propane (10 g, 44.65 mmol, 5.24 mL, 1 eq) and PPh₃ (35.13 g, 133.95 mmol, 3 eq) in toluene (40 mL) was stirred at 110° C. for 12 hours under N₂. The solid precipitated from the reaction mixture. The mixture was cooled to 0-5° C., the mixture was filtered and the filter cake was washed by PE (50 mL*2), the filter cake was concentrated in vacuum to give the compound triphenyl(3,3,3-trifluoropropyl)phosphonium iodide (35 g, 71.98 mmol, 80.60% yield) as white solid.

### Step 2

To a solution of 5-benzyloxypentan-1-ol (5 g, 25.74 mmol, 1 eq) in DCM (1250 mL) was added PCC (8.32 g, 38.61 mmol, 1.5 eq) and stirred at 25° C. for 3 h. The reaction mixture was diluted with MTBE (800 mL) stirred at 25° C. for 1 h, filtered through a pad of celite and silica gel (1:1). The filtrate was concentrated to give the compound 5-benzyloxypentanal (5 g, crude) as yellow oil.

### Step 3

To a mixture of triphenyl(3,3,3-trifluoropropyl)phosphonium iodide (13.15 g, 27.05 mmol, 1.3 eq) in THF (180 mL) was added KHMDS (1 M, 26.01 mL, 1.25 eq) dropwise at 0° C. under N2. The color was changed from white to green. The mixture was stirred at 25° C. for 30 min. Then the mixture was cooled to 0° C. and a solution of 5-benzyloxypentanal (4 g, 20.81 mmol, 1 eq) in THF (20 mL) was added dropwise at 0° C. The mixture was stirred at 0° C. for 30 min. The mixture was quenched by sat. NH₄Cl (150 mL) and extracted with ethyl acetate (100 mL*2). The combined organic phase was washed with brine (50 mL), dried with anhydrous Na₂SO₄, filtered and concentrated in vacuum to give the compound [(E)-8,8,8-trifluorooct-5-enoxy]methylbenzene (5.1 g) as colorless oil.

### Step 4

To a solution of [(E)-8,8,8-trifluorooct-5-enoxy]methylbenzene (4 g, 14.69 mmol, 1 eq) in EtOH (160 mL) and AcOH (1 mL) was added Pd(OH)₂/C (1 g, 10% purity) under N₂. The suspension was degassed under vacuum and purged with H₂ three times. The mixture was stirred under H₂ (50 psi) at 50° C. for 12 hours. TLC showed the starting material was consumed completely and one new spot formed. The reaction mixture was filtered by celite and the filtrate was concentrated in vacuum to give the compound 8,8,8-trifluorooctan-1-ol (crude) (2 g) as colorless oil.

### Step 5

To a solution of 8,8,8-trifluorooctan-1-ol (0.5 g, 2.71 mmol, 1 eq) in DMF (9 mL) was added PDC (3.06 g, 8.14 mmol, 3 eq) and stirred at 25° C. for 12 h. The reaction mixture was added water (20 mL) and extracted with ethyl acetate (20 mL*2). The combined organic phase was washed with sat. NaHCO₃ (20 mL*2). The combined aqueous phase was acidified with 4N HCl (50 mL) to pH=3, aqueous phase was extracted with ethyl acetate (20 mL*2). The combined organic phase was washed with brine (40 mL), dried with anhydrous Na₂SO₄, filtered and concentrated in vacuum to give the compound 8,8,8-trifluorooctanoic acid (250 mg, 1.17 mmol, 43.17% yield, 92.9% purity) as yellow oil.

### Step 6

A mixture of 6,6,6-trifluorohexanoic acid (400 mg, 2.35 mmol, 1 eq), (2R)-2-amino-N-[2-(4-hydroxyphenyl)ethyl]butanediamide (1.30 g, 2.82 mmol, 79% purity, 1.20 eq, TFA), HATU (1.07 g, 2.82 mmol, 1.2 eq) in DMF (3 mL) was added DIEA (668.50 mg, 5.17 mmol, 900.94 uL, 2.2 eq) at 0° C. and was stirred at 25° C. for 5 hr under N₂ atmosphere. The mixture was diluted with ACN (3 mL) and filtered. The filtrate was concentrated under reduced pressure to give a residue. The residue was purified by prep-HPLC (column: Waters Xbridge Prep OBD C18 150*40 mm*10 um; mobile phase: [water(10 mM NH4HCO3)-ACN]; B %: 10%-30%, 8 min) to give the compound (2R)-N-[2-(4-hydroxyphenyl)ethyl]-2-(6,6,6-trifluorohexanoylamino) butanediamide (100 mg, 245.42 umol, 10.44% yield, 99% purity) as white solid.

### Example 265: (R)-N′-(2,6-difluoro-4-hydroxyphenethyl)-2-octanamidosuccinamide

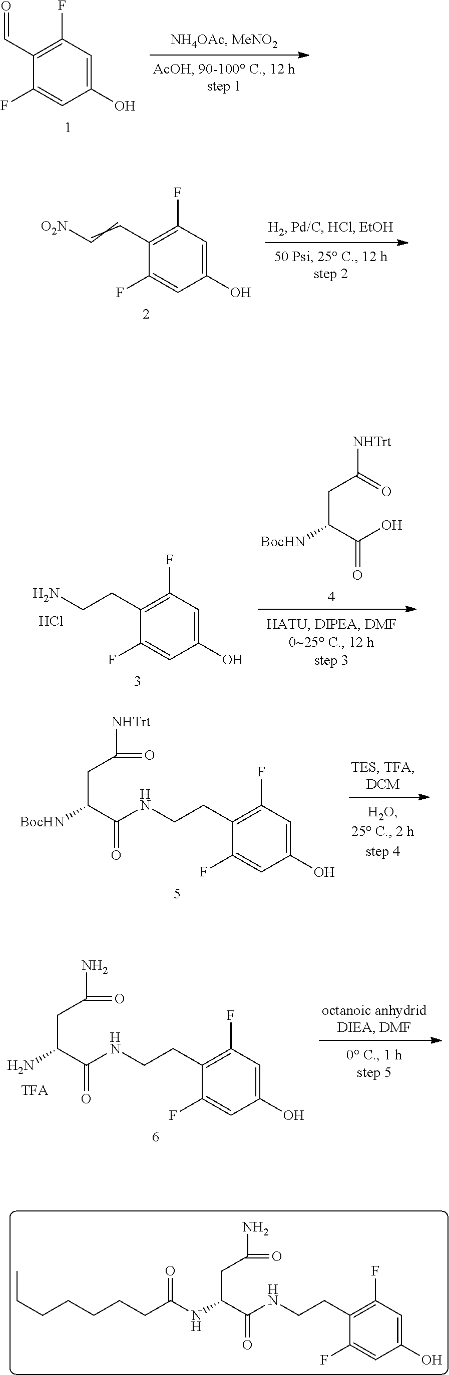

### Step 1

To a solution of NH₄OAc (3.05 g, 39.53 mmol, 2.5 eq) in AcOH (70 mL) and nitromethane (30.87 g, 505.73 mmol, 27.32 mL, 31.98 eq) was added 2,6-difluoro-4-hydroxy-benzaldehyde (2.5 g, 15.81 mmol, 1 eq) at 90° C., and stirred at 100° C. for 12 h. The reaction mixture was cooled to 15° C. and quenched with H₂O (200 mL) and sat·NaHCO₃ (500 mL), then extracted with ethyl acetate (100 mL*3), and the organic extracts was dried with Na₂SO₄, filtered and concentrated the filtrate in vacuo to give the 3,5-difluoro-4-[(E)-2-nitrovinyl]phenol (3 g, crude) as yellow oil.

### Step 2

To a solution of 10% Pd/C (400 mg) and HCl (12 M, 207.17 uL, 1 eq) in EtOH (20 mL) was added 3,5-difluoro-4-[(E)-2-nitrovinyl]phenol (500 mg, 2.49 mmol, 1 eq), stirred at 25° C. for 12 h under H₂ under 50 psi. The mixture was filtered and the filtrate was concentrated in vacuum to give a residue. The residue was triturated with EtOAc (10 mL) at 25° C. for 15 min and then filtered, the solid was concentrated under reduced pressure to give the compound 4-(2-aminoethyl)-3,5-difluoro-phenol (240 mg, 1.14 mmol, 46.06% yield, HCl) as a gray white solid.

### Step 3

To a solution of (2R)-2-(tert-butoxycarbonylamino)-4-oxo-4-(tritylamino)butanoic acid (440 mg, 927.20 umol, 1 eq) and 4-(2-aminoethyl)-3,5-difluoro-phenol (233.23 mg, 1.11 mmol, 1.2 eq, HCl) in DMF (5 mL) was added HATU (423.06 mg, 1.11 mmol, 1.2 eq) and DIPEA (359.49 mg, 2.78 mmol, 484.49 uL, 3 eq) at 0° C. The mixture was stirred at 25° C. for 12 h. The mixture was poured into H₂O (10 mL), then extracted with EtOAc (10 mL*3). The combined organic layer was washed with brine (10 mL*2), dried over Na₂SO₄, filtered and concentrated the filtrate under reduced pressure. The residue was purified by column chromatography (SiO₂, Petroleum ether/Ethyl acetate=10 to 3/1) to give the tert-butyl N-[(1R)-1-[2-(2,6-difluoro-4-hydroxy-phenyl)ethyl carbamoyl]-3-oxo-3-(tritylamino)propyl]carbamate (400 mg, 635.23 umol, 68.51% yield) as a brown solid.

### Step 4

The tert-butyl N-[(1R)-1-[2-(2,6-difluoro-4-hydroxy-phenyl)ethylcarbamoyl]-3-oxo-3-(tritylamino) propyl]carbamate (400 mg, 635.23 umol, 1 eq) was dissolved with TES (0.5 mL), H₂O (0.5 mL), DCM (1 mL) and TFA (18 mL), and stirred at 25° C. for 2 h. The mixture was concentrated under reduced pressure. (2R)-2-amino-N-[2-(2,6-difluoro-4-hydroxy-phenyl)ethyl]butanediamide (254 mg, crude, TFA) was obtained as yellow oil.

### Step 5

To a solution of octanoyl octanoate (134.77 mg, 498.40 umol, 1 eq) and (2R)-2-amino-N-[2-(2,6-difluoro-4-hydroxy-phenyl)ethyl]butanediamide (200 mg, 498.40 umol, 1 eq, TFA) in DMF (3 mL) was added DIPEA (193.24 mg, 1.50 mmol, 260.43 uL, 3 eq) and the mixture was stirred at 0° C. for 1 hr. The mixture was concentrated under reduced pressure to give a residue. The residue was purified by prep-HPLC (column: Welch Xtimate C18 100*25 mm*3 um; mobile phase: [water(0.05% HCl)-ACN]; B %: 35%-35%, 8 min) to give the compound (2R)-N-[2-(2,6-difluoro-4-hydroxy-phenyl)ethyl]-2-(octanoylamino) butanediamide (125 mg, 302.33 umol, 60.66% yield) as a white solid.

### Example 266: (R)-2-dodecanamido-N′-(4-hydroxyphenethyl)succinamide

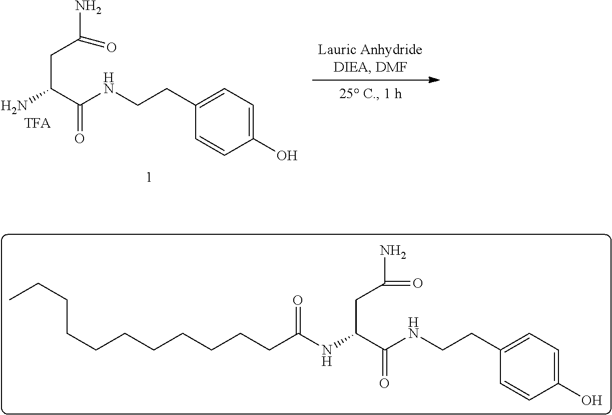 [SMILES: NC(=O)C[C@@H](N)C(=O)NCCc1ccc(O)cc1 ; CCCCCCCCCCCC(=O)N[C@H](CC(N)=O)C(=O)NCCc1ccc(O)cc1]

To a solution of (2R)-2-amino-N-[2-(4-hydroxyphenyl)ethyl]butanediamide (200 mg, 547.49 umol, 1 eq, TFA) and dodecanoyl dodecanoate (314.22 mg, 821.23 umol, 1.5 eq) in DMF (5 mL) was added DIPEA (212.28 mg, 1.64 mmol, 286.09 uL, 3 eq). The mixture was stirred at 25° C. for 1 hr. The mixture was concentrated under reduced pressure to give a residue. The residue was purified by prep-HPLC (column: Welch Xtimate C18 100*25 mm*3 um; mobile phase: [water(0.05% HCl)-ACN]; B %:30%-60%, 8 min) to give the compound (2R)-2-(dodecanoylamino)-N-[2-(4-hydroxyl phenyl)ethyl]butanediamide (47 mg, 104.06 umol, 19.01% yield, 96% purity) as a white solid.

### Example 267: (R)-N′-(4-hydroxyphenethyl)-2-(4-(1-methylpiperidin-4-yl)butanamido)succinamide

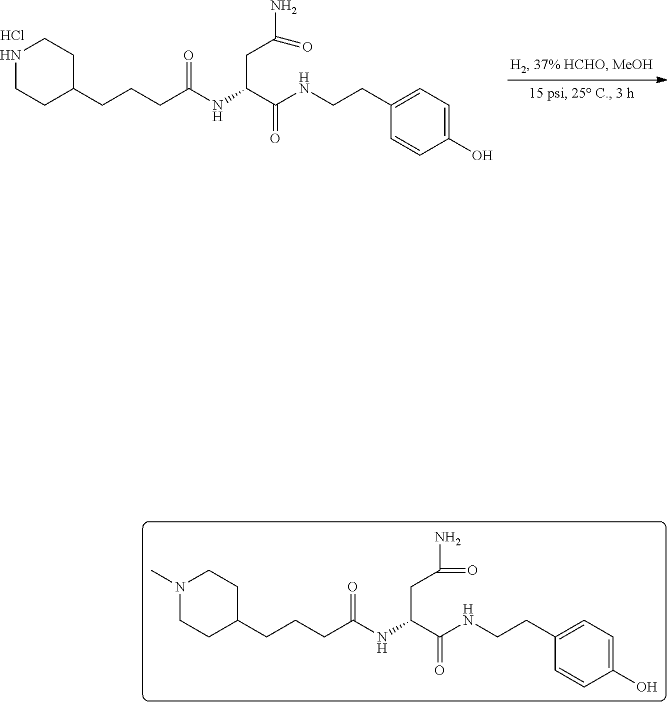 [SMILES: Cl ; NC(=O)C[C@@H](NC(=O)CCCC1CCNCC1)C(=O)NCCc1ccc(O)cc1 ; CN1CCC(CCCC(=O)N[C@H](CC(N)=O)C(=O)NCCc2ccc(O)cc2)CC1]

A mixture of (2R)-N-[2-(4-hydroxyphenyl)ethyl]-2-[4-(4-piperidyl)butanoylamino]butanediamide (50 mg, 113.39 umol, 1 eq, HCl), HCHO 37% aq. (36.81 mg, 453.55 umol, 33.77 uL, 4 eq), and 10% Pd/C (0.1 g,) in MeOH (5 mL) was degassed and purged with H₂ for 3 times, and then the mixture was stirred at 25° C. for 3 hr under H₂ atmosphere. The reaction mixture was filtered and concentrated under reduced pressure to give a residue. The residue was purified by prep-HPLC (column: Phenomenex Gemini-NX C18 75*30 mm*3 um; mobile phase: [water(10 mM NH₄HCO₃)-ACN]; B %: 1%-30%, 12 min) to give the compound (2R)-N-[2-(4-hydroxyphenyl) ethyl]-2-[4-(1-methyl-4-piperidyl)butanoylamino]butanediamide (1.8 mg, 4.21 umol, 3.52% yield, 97.8% purity) as a white solid.

### Example 268: (R)-2-decanamido-N1-(2-(pyridin-2-yl)ethyl)succinamide

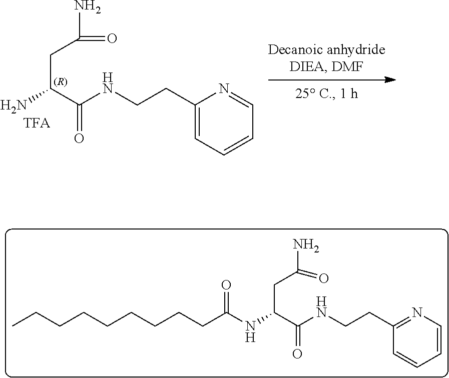

To a mixture of (2R)-2-amino-N-[2-(2-pyridyl)ethyl]butanediamide (400 mg, TFA) and decanoyl decanoate (241.60 mg, 739.95 umol, 1.2 eq) in DMF (12 mL) was added DIPEA (557.85 mg, 4.32 mmol, 751.82 uL, 7 eq) under N₂. The mixture was stirred at 25° C. for 1 hour. To the mixture was added H₂O (20 mL) slowly, then the precipitate was filtered and the filter cake was washed by H₂O (30 mL). The filter cake was concentrated in vacuum. The residue was purified by prep-HPLC (column: Phenomenex Gemini-NX 80*40 mm*3 um; mobile phase: [water(10 mM NH₄HCO₃)-ACN]; B %: 15%-55%, 8 min) to give (2R)-2-(decanoylamino)-N-[2-(2-pyridyl)ethyl]butanediamide (46 mg, 96.95% purity) as a white solid.

### Example 269: (R)-2-dodecanamido-N1-(2-(pyridin-2-yl)ethyl)succinamide

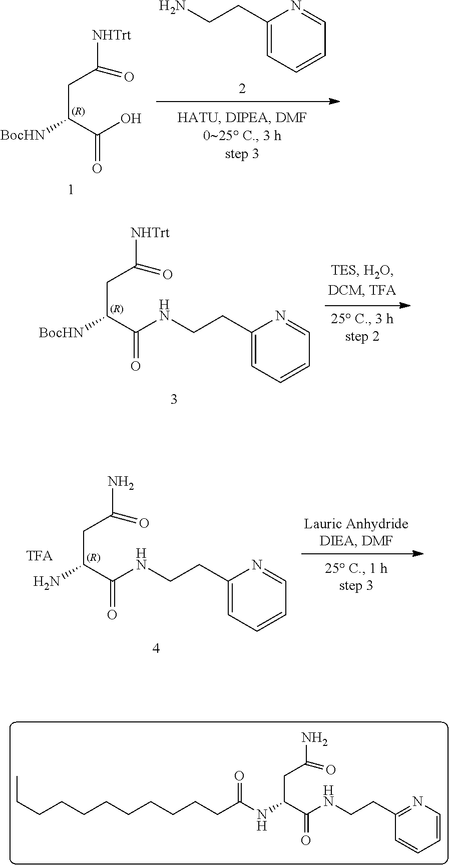

### Step 1

To a mixture of (2R)-2-(tert-butoxycarbonylamino)-4-oxo-4-(tritylamino)butanoic acid (1.5 g, 3.16 mmol, 1 eq) and 2-(2-pyridyl)ethanamine (463.39 mg, 3.79 mmol, 454.31 uL, 1.2 eq) in DMF (45 mL) was added HATU (1.44 g, 3.79 mmol, 1.2 eq) and DIPEA (898.74 mg, 6.95 mmol, 1.21 mL, 2.2 eq) at 0° C. under N2. The mixture was stirred at 25° C. for 3 hours. LCMS showed the reaction was completed and desired mass was detected. H₂O (90 mL) was added slowly to the mixture at 0° C., then filtered and the filter cake was washed by water (30 mL). The filter cake was concentrated in vacuum to give tert-butyl N-[(1R)-3-oxo-1-[2-(2-pyridyl)ethylcarbamoyl]-3-(tritylamino)propyl]carbamate (1.7 g, 98% purity) as a white solid.

### Step 2

A mixture of tert-butyl N-[(1R)-3-oxo-1-[2-(2-pyridyl)ethylcarbamoyl]-3-(tritylamino)propyl]carbamate (1.5 g, 2.59 mmol, 1 eq) in TFA (41.58 g, 364.67 mmol, 27.00 mL, 140.69 eq), DCM (3.96 g, 46.63 mmol, 3.00 mL, 17.99 eq), H₂O (1.50 g, 83.24 mmol, 1.50 mL, 32.11 eq) and triethylsilane (1.09 g, 9.39 mmol, 1.50 mL, 3.62 eq) was stirred at 25° C. for 3 h. The mixture was concentrated in vacuum to get (2R)-2-amino-N-[2-(2-pyridyl)ethyl]butanediamide (3 g, crude, TFA) as a yellow solid.

### Step 3

To a mixture of (2R)-2-amino-N-[2-(2-pyridyl)ethyl]butanediamide (400 mg, TFA) and dodecanoyl dodecanoate (283.12 mg, 739.95 umol, 1.2 eq) in DMF (12 mL) was added DIPEA (557.86 mg, 4.32 mmol, 751.84 uL, 7 eq) in one portion at 25° C. under N₂. The mixture was stirred at 25° C. for 1 hour. LCMS showed the reaction was completed. To the mixture was added water (20 mL) slowly, then the precipitate was filtered and the filter cake was washed by water (30 mL). The filter cake was concentrated in vacuum. The residue was purified by p-HPLC (column: Phenomenex Gemini-NX 80*40 mm*3 um; mobile phase: [water(10 mM NH₄HCO₃)-ACN]; B %: 20%-60%, 8 min) to give (2R)-2-(dodecanoylamino)-N-[2-(2-pyridyl) ethyl]butanediamide (7.3 mg, 96.93% purity) as a white solid.

### Example 270: (R)-N′-(4-hydroxyphenethyl)-2-methyl-2-octanamidosuccinamide

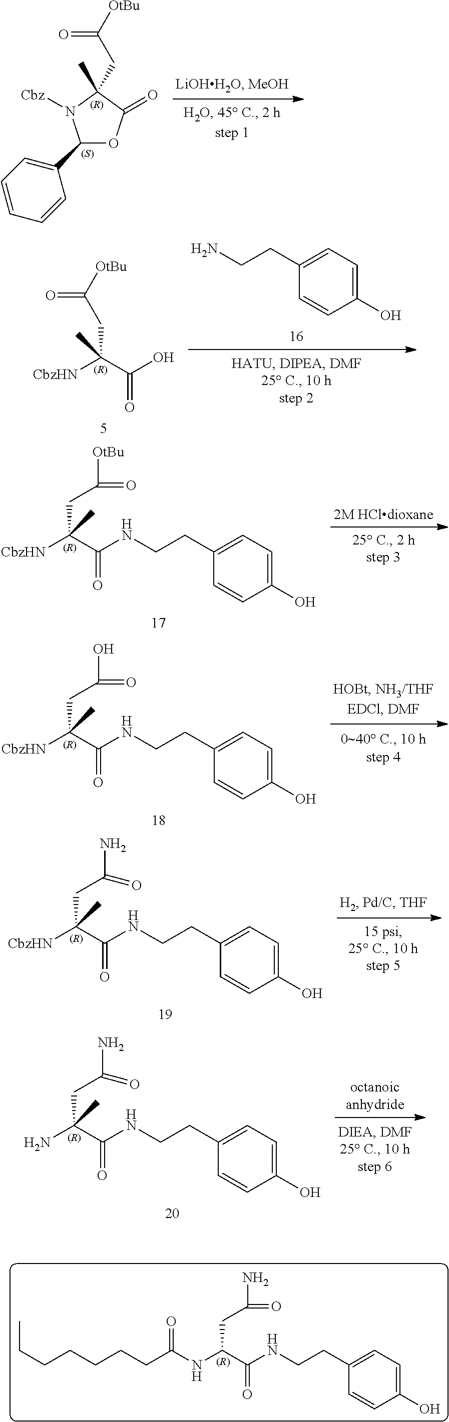

### Step 1

A mixture of benzyl (2S,4R)-4-(2-tert-butoxy-2-oxo-ethyl)-4-methyl-5-oxo-2-phenyl-oxazolidine-3-carboxylate (2 g, 4.70 mmol, 1 eq) in MeOH (4.7 mL) was added LiOH·H₂O (2 M, 4.70 mL, 2 eq) and the mixture was stirred at 45° C. for 2 hr under N2 atmosphere. The reaction mixture was diluted with H₂O (20 mL) and washed with EtOAc (60 mL, 20 mL*3). The aqueous phase was adjusted to pH=3 with aqueous 1N HCl. Then the aqueous phase was extracted with EtOAc (60 mL, 30 mL*2). The combined organic layers were dried over Na₂SO₄, filtered and the filtrate was concentrated under reduced pressure to give a residue. The residue was purified by column chromatography (SiO₂, Petroleum ether/Ethyl acetate=10/1 to 5/1) to give the compound (2R)-2-(benzyloxycarbonylamino)-4-tert-butoxy-2-methyl-4-oxo-butanoic acid (1 g, 2.82 mmol, 59.91% yield, 95% purity) as white solid.

### Step 2

A mixture of (2R)-2-(benzyloxycarbonylamino)-4-tert-butoxy-2-methyl-4-oxo-butanoic acid (0.4 g, 1.19 mmol, 1 eq), 4-(2-aminoethyl)phenol (195.18 mg, 1.42 mmol, 268.00 uL, 1.2 eq), HATU (540.99 mg, 1.42 mmol, 1.2 eq), DIEA (337.11 mg, 2.61 mmol, 454.33 uL, 2.2 eq) in DMF (3 mL) was stirred at 25° C. for 10 hr. The combined organic phase was diluted with EtOAc (20 mL) and washed with water (60 mL, 20 mL*3) and brine (40 mL, 20 mL*2), dried with anhydrous Na₂SO₄, filtered and the filtrate was concentrated under reduced pressure to give a residue. The residue was purified by column chromatography (SiO₂, Petroleum ether/Ethyl acetate=10/1 to 4/1) to give the compound tert-butyl (3R)-3-(benzyloxycarbonylamino)-4-[2-(4-hydroxyphenyl)ethylamino]-3-methyl-4-oxo-butanoate (0.32 g, 686.92 umol, 57.94% yield, 98% purity) as white solid.

### Step 3

A mixture of tert-butyl (3R)-3-(benzyloxycarbonylamino)-4-[2-(4-hydroxyphenyl)ethylamino]-3-methyl-4-oxo-butanoate (0.32 g, 700.94 umol, 1 eq) in HCl/dioxane (2 M, 7.58 mL, 21.63 eq) was stirred at 25° C. for 2 hr under N₂ atmosphere. The reaction mixture was concentrated to give the compound (3R)-3-(benzyloxycarbonylamino)-4-[2-(4-hydroxyphenyl)ethylamino]-3-methyl-4-oxo-butanoic acid (0.3 g, crude) as white solid.

### Step 4

A mixture of (3R)-3-(benzyloxycarbonylamino)-4-[2-(4-hydroxyphenyl)ethylamino]-3-methyl-4-oxo-butanoic acid (0.3 g, 749.21 umol, 1 eq), HOBt (121.48 mg, 899.05 umol, 1.2 eq), EDCI (172.35 mg, 899.05 umol, 1.2 eq) in DMF (2 mL) was degassed and purged with N₂ for 3 times and stirred at 0° C. for 30 min. The NH₃·THF (9.4 M, 1 mL, 12.55 eq) was added to the mixture and the mixture was stirred at 40° C. for 9.5 hr under N₂ atmosphere. The reaction mixture was concentrated under reduced pressure to get a residue. The residue was purified by column chromatography (SiO₂, Petroleum ether/Ethyl acetate=3/1 to 0/1) to give the compound benzyl N-[(1R)-3-amino-1-[2-(4-hydroxyphenyl)ethylcarbamoyl]-1-methyl-3-oxo-propyl]carbamate (0.1 g, 245.34 umol, 16.37% yield, 98% purity) as white solid.

### Step 5

To a solution of benzyl N-[(1R)-3-amino-1-[2-(4-hydroxyphenyl)ethylcarbamoyl]-1-methyl-3-oxo-propyl]carbamate (0.1 g, 250.35 umol, 1 eq) in THF (3 mL) was added 10% Pd/C (70 mg,) under N₂. The suspension was degassed under vacuum and purged with H₂ several times. The mixture was stirred under H₂ (15 psi) at 25° C. for 10 hr. The mixture was filtered through celite and the solvent was removed under reduced pressure to give the compound (2R)-2-amino-N-[2-(4-hydroxyphenyl)ethyl]-2-methyl-butanediamide (80 mg, crude) as colourless oil.

### Step 6

A mixture of (2R)-2-amino-N-[2-(4-hydroxyphenyl)ethyl]-2-methyl-butanediamide (0.08 g, 301.54 umol, 1 eq) in DMF (2 mL) was added DIEA (38.97 mg, 301.54 umol, 52.52 uL, 1 eq), octanoyl octanoate (73.38 mg, 271.38 umol, 0.9 eq) and was stirred at 25° C. for 10 hr under N₂ atmosphere. The reaction was diluted with EtOAc (20 mL) and washed with water (60 mL, 20 mL*3) and brine (40 mL, 20 mL*2), dried with anhydrous Na₂SO₄, filtered and concentrated under reduced pressure to give a residue. The residue was purified by prep-HPLC (column: Welch Xtimate C18 100*25 mm*3 um; mobile phase: [water(0.05% HCl)-ACN]; B %: 15%-45%, 8 min) to give the compound (2R)-N-[2-(4-hydroxyphenyl)ethyl]-2-methyl-2-(octanoylamino)butanedi amide (45 mg, 109.19 umol, 36.21% yield, 95% purity) as white solid.

### Example 271: (R)-N-(3-amino-1-(1-benzyl-1H-1,2,4-triazol-5-yl)-3-oxopropyl)octanamide

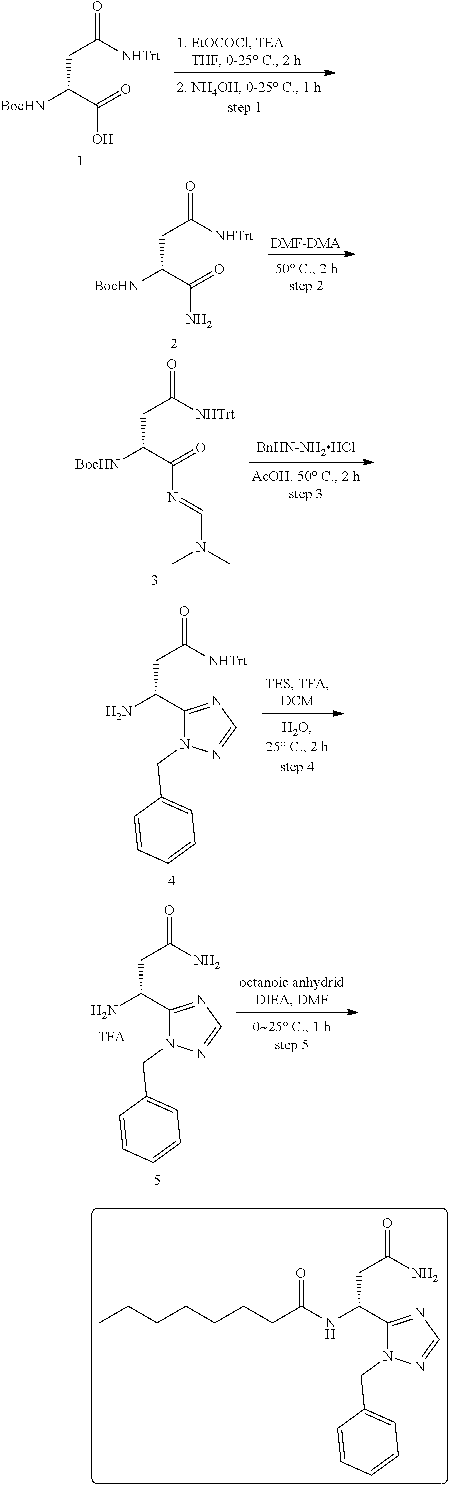

### Step 1

To a solution of (2R)-2-(tert-butoxycarbonylamino)-4-oxo-4-(tritylamino)butanoic acid (2 g, 4.21 mmol, 1 eq) in THF (40 mL) was added ethyl chloroformate (0.89 g, 8.20 mmol, 780.70 uL, 1.95 eq) and TEA (639.70 mg, 6.32 mmol, 879.92 uL, 1.5 eq) at 0° C., and then stirred at 25° C. for 2 h. The reaction was cooled to 0° C., then NH₃·H₂O (3.64 g, 25.97 mmol, 4.00 mL, 25%, 6.16 eq) was added into the mixture and stirred at 25° C. for 1 h. The solvent was removed under reduced pressure, the residue was washed with H₂O (20 mL) then filtered and the filter cake was concentrated under reduced pressure to give the tert-butyl N-[(1R)-1-carbamoyl-3-oxo-3-(tritylamino)propyl]carbamate (1.9 g, crude) as a white solid.

### Step 2

A solution of tert-butyl N-[(1R)-1-carbamoyl-3-oxo-3-(tritylamino)propyl]carbamate (1.9 g, 4.01 mmol, 1 eq) in DMF-DMA (20 mL) was stirred at 50° C. for 2 h. The mixture was poured into H₂O (50 mL), the solid was concentrated under reduce pressure to give the compound tert-butyl N-[(1R)-1-[(E)-dimethylaminomethylenecarbamoyl]-3-oxo-3-(tritylamino) propyl]carbamate (1.5 g, 2.70 mmol, 67.19% yield) as a white solid.

### Step 3

A solution of tert-butyl N-[(1R)-1-[(E)-dimethylaminomethylenecarbamoyl]-3-oxo-3-(tritylamino)propyl]carbamate (1.5 g, 2.84 mmol, 1 eq) and benzylhydrazine dihydrochloride (664.27 mg, 3.40 mmol, 1.2 eq) in AcOH (30 mL) was stirred at 50° C. for 2 h. The solvent was removed under reduced pressure, and the product was diluted H₂O (2 mL). The pH was adjusted to 7 with NH₃·H₂O, and the aqueous phase was extracted with EtOAc (10 mL*3). The combined organics were dried over sodium sulfate, filtered, and concentrated under reduced pressure. The residue was purified by column chromatography (SiO₂, Petroleum ether/Ethyl acetate=20/1 to 0/1) to give the compound (R)-3-amino-3-(1-benzyl-1H-1,2,4-triazol-5-yl)-N-tritylpropanamide (400 mg, 820.36 umol, 28.91% yield) as a white solid.

### Step 4

(R)-3-amino-3-(1-benzyl-1H-1,2,4-triazol-5-yl)-N-tritylpropanamide (100 mg, 205.09 umol, 1 eq) was dissolved with TES (0.5 mL), H₂O (0.5 mL), DCM (1 mL) and TFA (18 mL), stirred at 25° C. for 2 h. The mixture was concentrated under reduced pressure. (R)-3-amino-3-(1-benzyl-1H-1,2,4-triazol-5-yl)propanamide (50 mg, crude, TFA) was obtained as yellow oil.

### Step 5

To a solution of octanoyl octanoate (41.39 mg, 153.07 umol, 1.1 eq) and (R)-3-amino-3-(1-benzyl-1H-1,2,4-triazol-5-yl)propanamide (50 mg, 139.16 umol, 1 eq, TFA) in DMF (3 mL) was added DIPEA (53.95 mg, 417.48 umol, 72.71 uL, 3 eq) at 0° C. The mixture was stirred at 25° C. for 1 hr. The mixture was poured into H₂O (10 mL), filtered and the solid was concentrated under reduced pressure. The crude product was triturated with PE/EA=20/1 (10 mL) at 25° C. for 10 min, then the solid was filtered and concentrated under reduced pressure to give the compound N-[(1R)-3-amino-1-(2-benzyl-1,2,4-triazol-3-yl)-3-oxo-propyl]octanamide (19 mg, 51.15 umol, 36.76% yield) as a gray solid.

### Example 272: Isolation of ClbP for biochemical assays

The soluble peptidase domain (amino acids 38 to 375) of ClbP or the catalytically inert mutant S95A was cloned into pET28a using as a starting point previously defined boundaries such as those in Brotherton, J. Am. Chem. Soc., 135:3359-3362, 20 2013.

WT ClbP or S95A ClbP was isolated as an N-terminally tagged His5-SLJMO1-fusion protein from pET28b using E. coli B121 (DE3)RIPl to express the protein. The cells were lysed in 30 mM Tris pH 8.0, 300 mM NaCl, 10% (v/v) glycerol, benzonase (Sigma-Millipore), lysozyme, B-per (Thermo Fisher), and Yper (ThermoFisher). The lysate was clarified via centrifugation (13,000 g, 20 min), and the supernatant was incubated with HiBond Ni-NTA agarose (5 ml). The resin was subsequently washed with wash buffer (30 mM Tris pH 7.5, 250 mM NaCl, 10 mM imidazole, 3% (v/v) glycerol, and 2 mM TCEP). The resin was incubated at 30° C. for 1 h with 1.4 mg UlP1 protease. The resin was then washed with an additional fraction of wash buffer. Centrifuge filtration was used to concentrate the protein, and the concentrated protein was subsequently applied to a 16/600 Superdex 200 size exclusion column with an isocratic elution of 25 mM HEPES, 25 mM Tris pH 7.4, 325 mM NaCl, 10 mM KCl and 3% (v/v) glycerol at a flow rate of X ml/min. The fractions containing the protein were determined by SDS-PAGE, combined, and concentrated by centrifuge filtration. 3 mM TCEP was added to the concentrated protein, which was then aliquoted, flash frozen in liquid N₂, and stored at −80° C.

### Example 273: Assay with purified ClbP protein

To assess the activity of the purified enzyme, the purified protein or its catalytically inactive variant was incubated with compound. As a control, the compound was also incubated with buffer alone. After a designated interval, the reaction was stopped by addition of organic solvent. LCMS analysis was used to determine the relative amounts of starting material and product in the reactions with the active enzyme, the inert enzyme, and the no-enzyme control.

ClbP was incubated with test compounds in an in vitro assay. In a typical assay, 0, 1, or 20 μM purified WT or S95A ClbP was incubated with 100 or 500 μM compound in reaction buffer (50 mM Tris-HCl pH 7.5, 37.5 mM NaCl) at room temperature for 24 h. The total volume of this reaction was 150 μl.

After 24 h, 2 volumes of acetonitrile (300 μl) was added to the reaction to quench the solution. The reaction was centrifuged (4,000 g, 15 min), and then the supernatant was analyzed by LCMS. 5 μl of the supernatant was injected onto an Acquity UPLC BEH C18 1.7 μm 2.1×100 column.

Buffer A was 0.1% (v/v) formic acid in water and buffer B was 0.1% (v/v) formic acid in acetonitrile. The flow rate was 0.5 ml/min. The initial % B was 10, which was held for 30 s then ramped up to 90% B over 3 min, held at 90% B for 1 min, ramped down to 10% B over 15 s, and then held at 10% B for 15 s. The UV-VIS spectrum from 210-400 nm was monitored with 3.6 nm resolution. Positive and negative mode ESI were monitored from 100-1000 m/z with a 20 V cone voltage at 5 Hz sampling frequency in centroid collection.

The relative starting material remaining in the WT ClbP reaction was determined by LCMS and then compared to the residual starting material in the no-enzyme and catalytically inert enzyme (S95A ClbP) reactions. In an ideal case, the amount of residual starting material in the WT ClbP reaction was <25% of the amount in the S95A ClbP and no-enzyme conditions. In an acceptable case, the amount of residual starting material in the WT ClbP reaction was 25-75% of the amount in the S95A ClbP and no enzyme conditions. In a non-preferred case, the amount of residual starting material in the WT ClbP reaction was >75% of the amount in the S95A ClbP and no-enzyme conditions.

Formation of the anticipated product was likewise quantified by LCMS analysis and confirmed by coelution with an external product standard. In an ideal case, the amount of product formed in the WT ClbP reaction corresponded to >75% of the theoretical maximum. In an acceptable case, the amount of product formed in the WT ClbP reaction corresponded to 25-75% of the theoretical maximum. In a nonpreferred case, the amount of product formed in the WT ClbP reaction corresponded to <25% of the theoretical maximum.

### Example 274: Cell-based assay with bacterial enzymes with an LCMS readout

A vector was designed to express an enzyme in its native form or a catalytically inactive version. The vector was transformed into a heterologous host, for example an E. coli expression strain. In the case of ClbP, a strain natively encoding ClbP such as E. coli CFT073 ATCC 700928 or E. coli Nissle 1917 was used. E. coli BL21 (DE3) does not encode ClbP and therefore was used as a negative control.

The heterologous host (containing the wild-type enzyme, a catalytically inert version, or the empty vector) or the native strain was grown to a desirable level.

Compound was added to the bacterial growth or uninoculated medium. The mixture was incubated for a designated period of time.

After incubation, the mixture was rendered compatible with LCMS analysis via addition of organic solvent or lyophilization followed by dissolution in organic solvent. The amount of residual starting material and the product was quantified by LCMS.

### Example 275: Cell-based assay with ClbP with an LCMS readout

ClbP was tested for its ability to activate compounds. Various test compounds were incubated with E. coli natively expressing full-length WT ClbP (E. coli CFT073 ATCC 700928 and/or E. coli Nissle 1917) or, as a negative control, an E. coli strain that does not encode ClbP (E. coli BL21 (DE3)). Each of these three strains was struck out onto an appropriate solid media such as LB agar or BHI agar. The bacteria were then grown aerobically overnight at 37° C.

A single colony was isolated from the plate and inoculated into 10 ml LB. The liquid cultures were grown overnight aerobically at 37° C. with rocking on a nutator.

The saturated E. coli cultures were transferred into a biosafety cabinet. 5 μL of each culture was inoculated into fresh LB broth (200 μL total, 1:40 (v/v) dilution of the overnight culture), and the tested compound was added to a final concentration of 10 μM. To establish the native stability of the compound in media alone, the compound was also added to uninoculated media at a final concentration of 10 μM. Each compound was tested in duplicate with each strain or the media-alone incubation. The plate was sealed and incubated aerobically on a nutator at 37° C. for 20 h.

After 20 h, the reactions were stopped by addition of 200 μL acetonitrile. The contents of each 40 well were mixed, and then the plate was centrifuged (4,000 rpm, 10 min). The plate was diluted serially to a final dilution of 1:100 first with Acetonitrile (10λ dilution) and then with (4:1) Mobile Phase A—20 mM ammonium formate+water/Mobile Phase B−Isopropanol: Methanol: Water (4:4:2)+20 mM ammonium formate (10λ dilution).

The amount of each remaining parent compound in each incubation was quantified by LC-MS analysis, with multiple-reaction monitoring targeting transitions specific to each parent compound. The amount of compound present in the media-alone condition was normalized to 100%. The amount of parent compound present in the reaction with the ClbP-encoding E. coli (E. coli CFT073 ATCC 700928 and/or E. coli Nissle 1917) was compared to the amount present in the media-alone and expressed as a percent. Likewise, the amount of parent compound present in the reaction with the negative control E. coli BL21 (DE3) was compared to the amount present in the media-alone and expressed as a percent.

In an ideal case, the amount of residual starting material in the reaction(s) with ClbP-encoding E. coli was <25% of the amount in the E. coli BL21 (DE3) and media-alone conditions. In an acceptable case, the amount of residual starting material in the reaction(s) with ClbP-encoding E. coli was 25-75% of the amount in the E. coli BL21 (DE3) and media-alone conditions. In a non-preferred case, the amount of residual starting material in the reaction(s) with ClbP-encoding E. coli was >75% of the amount in the E. coli BL21 (DE3) and media-alone conditions.

### Example 276: in vitro stability studies

### Assay 1. Stability of Compounds in Simulated Gastric Fluid (SGF)

This assay was used to assess the stability of a compound in a stomach.

Medium was prepared by dissolving 2 g of sodium chloride in 0.6 L in ultrapure water (MilliQ®, Millipore Sigma, Darmstadt, Germany). The pH was adjusted to 1.6 with 1N hydrochloric acid, and the volume was then adjusted to 1 L with purified water.

60 mg FaSSIF powder (Biorelevant™, London, UK) were dissolved in 500 mL buffer (above). Pepsin was added (0.1 mg/mL) (Millipore Sigma, Darmstadt, Germany), and the solution was stirred. The resulting SGF media were used fresh for each experiment.

Test compounds were dissolved in DMSO stock to 1 mM. An aliquot of the DMSO stock solution was removed and diluted in the SGF Media in 15 mL falcon tubes to generate a total compound concentration of 1 μM. A 1 mL aliquot was immediately removed and diluted once with 1 volume of acetonitrile for TO timepoint. The mixture was sealed and mixed at 37° C. in an incubator. Aliquots (1 mL) were removed at regular intervals and immediately quenched by the addition of 1 volume of acetonitrile. The resulting samples were analyzed by LCMS to determine degradation rates in SGF

### Assay 2. Stability of Compounds in Simulated Intestinal Fluid (SIF)

This assay was used to assess the stability of a compound in a small intestine.

Phosphate buffer was prepared by dissolving 0.42 g of sodium hydroxide pellets and 3.95 g of monobasic sodium phosphate monohydrate and 6.19 g of sodium chloride in ultrapure water (MilliQ®, Millipore Sigma, Darmstadt, Germany). The pH was adjusted to 6.7 using aq. HCl and aq. NaOH, as necessary, and the solution was diluted with ultrapure water to produce 1 L of the pH 6.7 buffer.

112 mg FaSSIF powder (Biorelevant™, London, UK) was dissolved in 50 mL of the pH 6.7 buffer. 2 to 3 mL of the resulting solution were then added to 500 mg pancreatin (Millipore Sigma, Darmstadt, Germany). The resulting mixture was agitated by finger tapping the vessel containing the mixture until milky suspension formed. At this time, the remainder of the 50 mL FaSSiF/pH 6.7 buffer solution was added. The resulting suspension was flipped upside down 10 times to produce SIF, which was used fresh.

Test compounds were dissolved in DMSO stock to 1 mM. An aliquot of the DMSO stock solution was removed and diluted in the SIF media in 15 mL falcon tubes to produce a mixture with a tested compound concentration of 1 μM. A 1 mL aliquot was immediately removed and diluted once with 1 volume of acetonitrile for TO timepoint. The mixture was sealed and agitated at 37° C. in an incubator. Aliquots (1 mL) were removed at regular intervals and immediately quenched by the addition of 1 volume of acetonitrile. The resulting samples were analyzed by LCMS to determine degradation rates.

### Example 277: Caco-2 permeability assay

Caco-2 Permeability/Uptake Assay

Days 1-21:

1) Caco-2 cells were maintained and fed on a Monday/Wednesday/Friday schedule in T75 flasks (Corning #431464U) in DMEM (Dulbeco's Modified Eagle Medium, Gibco #11965092) supplemented with 1×Modified Eagle Medium (MEM)/nonessential amino acids (NEAA; media was Gibco #1037088) and 10% heat inactivated FBS (Gibco #16000044). The flasks were split at less than 85% confluency and reseeded with 2 million cells per flask.

2) Assay plates were seeded at 75,000 cells per 100 μL apical well with 20 mL growth medium as above in the feeding chamber. Cells are grown at 37° C. 5% CO₂. Media was exchanged Monday/Wednesday/Friday for 21 days until day of assay.

Day 21:

1) Buffer solution was prepared in Hank's Balanced Salt Solution (HBSS) supplemented with Ca and Mg without phenol red (Gibco #14025-0092) and 10 mM HEPES (made from Gibco #15630080).

2) 21-day plates were removed from the incubator, washed 2× with above buffer in the apical well and feeding chambers and then blotted dry on paper towels.

3) Dosing solutions were prepared in above buffer at 5 μM final concentration test article.

4) Assay plates were dosed in the following orientation (below) with 60 μL dosing solution added to the apical side and 180 μL added to the basolateral side.

APICAL PLATE

| | 1 | 2 | 3 | 4 | 5 | 6 | 7 | 8 | 9 | 10 | 11 | 12 |
|---|---|---|---|---|---|---|---|---|---|---|---|---|
| A | CDP1 | CDP1 | CDP1 | BUFFER | BUFFER | BUFFER | CDP9 | CDP9 | CDP9 | BUFFER | BUFFER | BUFFER |
| B | CDP2 | CDP2 | CDP2 | BUFFER | BUFFER | BUFFER | CDP10 | CDP10 | CDP10 | BUFFER | BUFFER | BUFFER |
| C | CDP3 | CDP3 | CDP3 | BUFFER | BUFFER | BUFFER | CDP11 | CDP11 | CDP11 | BUFFER | BUFFER | BUFFER |
| D | CDP4 | CDP4 | CDP4 | BUFFER | BUFFER | BUFFER | CDP12 | CDP12 | CDP12 | BUFFER | BUFFER | BUFFER |
| E | CDP5 | CDP5 | CDP5 | BUFFER | BUFFER | BUFFER | CDP13 | CDP13 | CDP13 | BUFFER | BUFFER | BUFFER |
| F | CDP6 | CDP6 | CDP6 | BUFFER | BUFFER | BUFFER | CDP14 | CDP14 | CDP14 | BUFFER | BUFFER | BUFFER |
| G | CDP7 | CDP7 | CDP7 | BUFFER | BUFFER | BUFFER | CDP15 | CDP15 | CDP15 | BUFFER | BUFFER | BUFFER |
| H | CDP8 | CDP8 | CDP8 | BUFFER | BUFFER | BUFFER | CDP16 | CDP16 | CDP16 | BUFFER | BUFFER | BUFFER |

BASOLATERAL PLATE

| | 1 | 2 | 3 | 4 | 5 | 6 | 7 | 8 | 9 | 10 | 11 | 12 |
|---|---|---|---|---|---|---|---|---|---|---|---|---|
| A | BUFFER | BUFFER | BUFFER | CDP1 | CDP1 | CDP1 | BUFFER | BUFFER | BUFFER | CDP9 | CDP9 | CDP9 |
| B | BUFFER | BUFFER | BUFFER | CDP2 | CDP2 | CDP2 | BUFFER | BUFFER | BUFFER | CDP10 | CDP10 | CDP10 |
| C | BUFFER | BUFFER | BUFFER | CDP3 | CDP3 | CDP3 | BUFFER | BUFFER | BUFFER | CDP11 | CDP11 | CDP11 |
| D | BUFFER | BUFFER | BUFFER | CDP4 | CDP4 | CDP4 | BUFFER | BUFFER | BUFFER | CDP12 | CDP12 | CDP12 |
| E | BUFFER | BUFFER | BUFFER | CDP5 | CDP5 | CDP5 | BUFFER | BUFFER | BUFFER | CDP13 | CDP13 | CDP13 |
| F | BUFFER | BUFFER | BUFFER | CDP6 | CDP6 | CDP6 | BUFFER | BUFFER | BUFFER | CDP14 | CDP14 | CDP14 |
| G | BUFFER | BUFFER | BUFFER | CDP7 | CDP7 | CDP7 | BUFFER | BUFFER | BUFFER | CDP15 | CDP15 | CDP15 |
| H | BUFFER | BUFFER | BUFFER | CDP8 | CDP8 | CDP8 | BUFFER | BUFFER | BUFFER | CDP16 | CDP16 | CDP16 |

5) The plates were assembled and incubated at 37° C. in ambient air for 2 h with gentle rocking (˜1 rock every 2 seconds).

6) After 2 hours the plates were removed and disassembled. Sampling was as follows:
Receiver wells: 40 μL was added to 260 μL acetonitrile
Donor wells: 8 μL was added to 32 μL HBSS and then 260 μL acetonitrile for a ⅕
dilution. The dilution was performed to prevent saturation in LC/MS. Depending on
sensitivity, the dilution scheme can be adjusted.

7) A 6-point standard curve was prepared by threefold serial dilution of the dosing solution in HBSS. 40 μL of each standard was transferred into 260 μL acetonitrile.

8) Assay and standard curve plates were diluted 1:1 (v/v) into a 96 well plate containing acetonitrile supplemented with 0.1% (v/v) formic acid and 50 nM tolbutamide.

9) The concentrations of analytes were interpolated based on the standard curve for each analyte. Pₐₚₚ was calculated with the following concentration:
Vr=volume receiver and was 0.06 mL or 0.18 mL
A=Filter area (0.1 cm2)
T=incubation time (7200 s)
[Rec]=receiver concentration (calculated from the standard curve)
[Donor]=Donor concentration (5 μM)
Pₐₚₚ=((Vᵣ/(A*T))*([Rec]/[Donor])*1000000=Pₐₚₚ*10⁻⁶ cm/s
Pₐₚₚ ratio was calculated with:

P_{app,A-B}/P_{app,B-A}
% recovery was calculated as:
([calculated donor well]+(dilution*[rec well])/[actual donor])*100=% recovery

### Example 278: Solubility determination assay

The stock solutions of test compounds and control compound progesterone were prepared in DMSO at the concentrations of 10 mM. 15 μL of stock solution (10 mM) of each sample was placed in order into their proper 96-well rack. 485 μL of PBS pH 1.6 and pH 7.4 were added into each vial of the cap-less Solubility Sample plate. The assay was performed in singlet. One stir stick was added to each vial and then the vial was sealed using a molded PTFE/Silicone plug. The solubility sample plates were then transferred to the Eppendorf Thermomixer Comfort plate shaker and shaken at 25° C. at 1100 rpm for 2 hours. After completion of the 2 hours, plugs were removed, and the stir sticks were removed using a big magnet. The samples from the Solubility Sample plate were transferred into the filter plate. Using the Vacuum Manifold, all the samples were filtered. An aliquot of 5 μL was taken from the filtrate followed by addition of 495 μL of a mixture of H₂O and acetonitrile containing internal standard (1:1). A certain proportion of ultrapure water was used to dilute the diluent according to the peak shape. The dilution factor was changed according to the solubility values and the LC-MS signal response.

From the 10 mM DMSO STD plate, 6 μL was transferred into the remaining empty plate, and then 194 μL of DMSO were added to that plate to have a STD concentration of 300 μM. From the 300 μM DMSO STD plate, 5 μL were transferred into the remaining empty plate, and then 495 μL of a mixture of H₂O and acetonitrile containing internal standard (1:1) were added to that plate to have a final STD concentration of 3 μM. A certain proportion of ultrapure water was used to dilute the diluent according to the peak shape. The concentrations of the standard samples were changed according to the LC-MS signal response.

The plate was placed into the well plate autosampler. The samples were evaluated by LC-MS/MS analysis.

All calculations were carried out using Microsoft Excel.

The filtrate was analyzed and quantified against a standard of known concentration using LC coupled with mass spectral peak identification and quantitation. Solubility values of the test compound and control compound were calculated as follows:

$$[ Sample ] = \frac{Area ratio Sample \times INJ VOL STD \times DF Sample \times [ S T D ]}{Area ratio STD \times INJ VOL Sample}$$

Any value of the compounds that was not within the specified limits was rejected and the experiment was repeated.

### Example 279: Growth and concentration of bacterial strains

E. coli Nissle 1917 and E. coli BL21(DE3) were streaked out onto a BHI or LB plate and grown aerobically overnight at 37° C. to select for individual colonies. The day before the assay, a single colony of each strain was inoculated into 30 mL LB and grown aerobically overnight at 37° C. on a nutator. This saturated overnight culture was centrifuged to create a firm pellet (3,200 g, 10 min, 4° C.), and the supernatant was removed. The pellet was resuspended in 30 mL sterile PBS under sterile conditions, and the cells were again centrifuged (3,200 g, 10 min, 4° C.). The supernatant was removed. The pellet was resuspended in 3 mL sterile PBS buffer to make the working solutions of E. coli cells.

### Example 280: Whole cell, cell lysate, and enzymatic fluorescence assay to generate an IC50 curve

Whole Cell Activity Assay

Day 1:

1) E. coli ClbP+ were taken from a frozen stock and streaked onto BHI plates. They were grown aerobically at 37° C. overnight in the incubator.

Day 2:

1) A single colony was selected from the plate and inoculated into 30 mL LB in a 50 mL conical vial eight times (240 mL total). The bacteria were grown aerobically overnight (16-20 h) at 37° C. on a nutator with rocking.

Day 3:

1) The saturated overnight culture was centrifuged to create a firm pellet (3,200 g, 10 min, 4° C.). In a biosafety cabinet, the supernatant was removed and then the pellet was resuspended in 30 mL sterile PBS.

2) The bacteria were centrifuged again (3,200 g, 10 min, 4° C.). The supernatant was removed, and the pellet was resuspended in sterile PBS buffer (6 mL) in each of the 8 tubes. This fivefold concentrated bacterial solution was the working solution.

3) The bacterial working solution (192 μL) was mixed with inhibitor (6 μL, final concentration was 300, 100, 33.3, 11.1, 3.7, 1.2, 0.4, or 0 μM). Fluorogenic substrate (2 μL, final concentration 5 μM) was immediately added across the plate to initiate the reaction. As a control, each plate contained Compound No. III in this dose response curve. The bacterial concentration was fivefold concentrated compared to the original saturated overnight culture. Carryover from DMSO was 4% (v/v). All compounds were tested in singlicate.

4) The plate was loaded into a spectrophotometer preheated to 37° C. The fluorescence was recorded every 2 min for 46 min. The excitation wavelength was 360 nm (20 nm bandwidth), the emission wavelength was 460 nm (20 nm bandwidth), the optics position was bottom, the gain was 65, the light source was a tungsten bulb, and the read speed was normal.

5) The linear portion of the fluorescence increase was 10-30 min. The rate of fluorescence increase (A.U./min) for each well in an inhibitor series was normalized to the 0 μM inhibitor condition. The normalized activity as a function of inhibitor concentration was then fit to a three parameter IC50 curve in Graphpad Prism.

The IC₅₀ levels from the whole cell activity assay of compounds of the disclosure are reported in Table 6 below. “A” indicates an IC₅₀ value less than 100 μM, “B” indicates an IC₅₀ between 100 and 300 μM, “C” indicates an IC₅₀ above 300 μM.

Cell Lysate Activity Assay

Day 1:

1) E. coli ClbP+ were taken from a frozen stock and streaked onto BHI plates. They were grown aerobically at 37° C. overnight in the incubator.

Day 2:

1) A single colony was selected from the plate and inoculated into 30 mL LB in a 50 mL conical vial eight times (240 mL total). The bacteria were grown aerobically overnight (16-20 h) at 37° C. on a nutator with rocking.

Day 3:

1) The saturated overnight culture was centrifuged to create a firm pellet (3,200 g, 10 min, 4° C.). The supernatant was removed and then the pellet was resuspended in 30 mL sterile PBS.

2) The bacteria were centrifuged again (3,200 g, 10 min, 4° C.). The supernatant was removed, and the pellet was resuspended in sterile PBS buffer (3 mL) in each 8 tubes to make a tenfold concentrated bacterial solution.

3) A lysozyme-EDTA (LE) solution was made in ddH2O (0.05 mg/mL lysozyme, 0.25 M EDTA).

4) For each of the eight tubes, LE solution (3 mL) was mixed with concentrated bacterial solution (3 mL). This mixture was incubated at 37° C. for 30 min to generate the lysates.

5) The lysates (192 μL) were mixed with inhibitor (6 μL, final concentration was 300, 100, 33.3, 11.1, 3.7, 1.2, 0.4, or 0 μM). Fluorogenic substrate (2 μL, final concentration 5 μM) was immediately added across the plate to initiate the reaction. As a control, each plate contained Compound No. III in this dose response curve. The final concentration of bacterial lysates was fivefold concentrated compared to the original saturated overnight culture. Carryover from DMSO was 4% (v/v). All compounds were tested in singlicate.

6) The plate was loaded into a spectrophotometer preheated to 37° C. The fluorescence was recorded every 2 min for 46 min. The excitation wavelength was 360 nm (20 nm bandwidth), the emission wavelength was 460 nm (20 nm bandwidth), the optics position was bottom, the gain was 65, the light source was a tungsten bulb, and the read speed was normal.

7) The linear portion of the fluorescence increase was 0-20 min. The rate of fluorescence increase (A.U./min) for each well in an inhibitor series was normalized to the 0 μM inhibitor condition. The normalized activity as a function of inhibitor concentration was then fit to a three parameter IC50 curve in Graphpad Prism.

The IC₅₀ levels from the cell lysate activity assay of compounds of the disclosure is reported in Table 6 below. “A” indicates an IC₅₀ value less than 100 μM, “B” indicates an IC₅₀ between 100 and 300 μM, “C” indicates an IC₅₀ above 300 μM.

ClbP Molecular (Purified Protein) Assay

1) Full-length WT ClbP was thawed on ice. A diluted enzyme solution (2 nM) was made by mixing 3.57 μL of enzyme with 20 mL of reaction buffer (50 mM Tris pH 8.0, 200 mM NaCl, 0.02% (w/v) n-dodecyl-b-D-maltoside (DDM)).

2) In a 96 well plate, the diluted protein solution (73.5 μL) was mixed with inhibitor (1.5 μL, of varying concentrations, see below) and preincubated for 15 min at room temperature. Then, reaction buffer (75 μL) containing 10 μM fluorogenic substrate was added to initiate the reaction. After addition of substrate, the final concentrations were 1 nM full-length WT ClbP, 5 μM fluorogenic substrate in 50 mM Tris pH 8.0, 200 mM NaCl, 0.02% (w/v) DDM, with either 0, 0.14, 0.41, 1.2, 3.7, 11, 33, or 100 μM inhibitor. For all conditions, the DMSO carryover was 2% (v/v) at the preincubation stage and 1% in the reaction stage. Compound No. III was always present as a control in each plate, and all compounds were tested in singlicate. In previous assays, the Km of the fluorogenic substrate was determined to be approximately 5 μM.

3) Immediately after addition of substrate to enzyme, the plate was transferred into a spectrophotometer held at room temperature. The fluorescence was recorded every 1.5 min for 15 min. The excitation wavelength was 360 nm (20 nm bandwidth), the emission wavelength was 440 nm (20 nm bandwidth), the optics position was bottom, the gain was extended, the light source was a xenon flash lamp, and the read speed was normal.

4) The linear portion of the fluorescence increase is at least from 0-6 min, so this time regime is used. The rate of fluorescence increase (A.U./min) for each well in an inhibitor series was normalized to the 0 μM inhibitor condition. The normalized activity as a function of inhibitor concentration was then fit to a three parameter IC50 curve in Graphpad Prism.

The IC₅₀ levels from the enzymatic inhibition activity assay of compounds of the disclosure is reported in Table 6 below. “A” indicates an IC₅₀ value less than 100 μM, “B” indicates an IC₅₀ between 100 and 300 μM, “C” indicates an IC₅₀ above 300 μM.

TABLE 6

| | ClbP Whole Cell | ClbP Lysate | ClbP Biochemical |
|---|---|---|---|
| Compound | Inhibition Assay: | Inhibition Assay: | Inhibition Assay: |
| No. | IC50 | IC50 | IC50 |
| III | A | A | A |
| IX | C | C | B |
| LXX | C | C | B |
| LXXVI | A | A | B |
| | A | A | not determined |
| VIII | A | A | A |
| XCIX | C | C | |
| XVI | C | A | A |
| | C | A | A |
| | not determined | not determined | A |
| XVIII | C | C | B |
| XIX | C | C | B |
| CC | A | A | A |
| CCI | A | A | A |
| CCIII | A | A | A |
| CCIV | C | C | B |
| CCV | A | A | A |
| CCVI | A | A | A |
| CCVII | C | C | A |
| CCVIII | A | A | A |
| CCIX | C | C | B |
| CCX | C | C | B |
| CCXI | C | C | B |
| CCXII | C | C | B |
| CCXIII | A | A | A |
| CCXIV | C | C | A |
| CCXIX | A | B | A |
| CCXL | A | A | A |
| CCXLI | A | A | A |
| CCXLII | A | A | A |
| CCXLIII | A | A | A |
| CCXLIV | A | A | A |
| CCXLIX | A | A | A |
| CCXLV | A | A | A |
| CCXLVI | A | B | A |
| CCXLVII | A | A | A |
| CCXLVIII | B | A | A |
| CCXVI | A | B | B |
| CCXVIII | A | A | B |
| | A | A | not determined |
| CCXX | A | A | A |
| | A | A | not determined |
| CCXXI | A | A | A |
| CCXXII | A | A | A |
| CCXXIII | A | A | A |
| CCXXIV | A | A | A |
| CCXXV | A | A | A |
| CCXXVI | A | A | A |
| CCXXVII | A | A | A |
| CCXXVIII | A | A | A |
| CCXXX | A | A | A |
| CCXXXI | A | A | A |
| CCXXXII | A | A | A |
| CCXXXIII | A | A | A |
| CCXXXIV | A | C | B |
| CCXXXV | A | C | A |
| CCXXXVI | C | A | A |
| CCXXXVII | A | A | A |
| CCXXXVIII | A | A | A |
| CCXXXIX | A | A | A |
| CCL | A | A | A |
| CCLI | A | B | A |
| CCLII | C | C | B |
| CCLIII | A | A | A |
| CCLIV | A | A | A |
| CCLV | A | A | A |
| CCLVI | A | A | A |
| CCLVII | C | C | B |
| CCLVIII | C | C | B |
| CCLIX | A | B | B |
| CCLX | A | A | A |
| CCLXI | A | A | A |
| CCLXII | C | C | A |
| CCLXIII | A | A | A |
| CCLXIV | C | C | B |
| CCLXV | C | C | B |
| CCLXVI | A | A | A |
| CCLXVII | A | B | A |
| CCLXVIII | A | C | B |
| CCLXIX | A | A | A |
| CCLXX | A | A | A |
| CCLXXI | B | C | A |
| CCLXXIII | C | C | B |
| CCLXXIV | A | A | A |
| CCLXXV | C | C | A |
| CCLXXVI | C | A | A |
| CCLXXVII | A | A | A |
| CCLXXX | C | C | B |
| CCLXXXI | C | C | B |
| CXCI | A | B | B |
| CXCII | C | A | B |
| CXC | C | C | B |
| CXCIV | A | A | A |
| CXCIX | A | A | A |
| CXCV | C | C | B |
| CXCVI | A | A | A |
| CXCVII | A | A | A |
| CXCVIII | A | B | A |
| CCLXXXIII | A | A | A |
| CCLXXXIV | B | B | A |
| CCLXXXV | B | B | A |
| CCLXXXVIII | B | B | A |
| CCLXXXIX | B | B | A |
| CCXC | A | A | A |
| CCXCI | A | A | A |
| CCXCII | A | A | A |
| CCXCIII | A | A | A |
| CCXCIV | A | A | A |
| CCXCV | A | A | A |
| CCXCVI | A | A | A |
| CCXCVII | A | A | A |
| CCXCVIII | A | A | A |
| CCXCIX | A | A | A |
| CCC | C | C | C |
| CCCI | A | A | A |
| CCCII | A | A | A |
| CCCIII | A | A | A |
| CCCIV | A | A | A |
| CCCV | A | A | A |
| CCCVI | A | A | A |
| CCCVII | A | A | A |
| CCCVIII | A | C | C |
| CCCIX | A | A | A |
| CCCX | C | A | A |
| CCCXI | C | C | C |
| CCCXII | C | C | C |

### Example 281: Inhibition in fecal+ assay

E. coli Nissle 1917 and E. coli BL21(DE3) are prepared as specified in Example 247. A ClbP-free fecal sample (as determined by whole genome sequencing) is transferred into an anaerobic chamber and thoroughly resuspended to make a fecal slurry (15% w/v) in PBS. The large particulate matter is allowed to settle for 15 min.

The fecal slurry is then combined with the working solution of E. coli to make seven different microbial consortia with different ratios of E. coli (ClbP+ or ClbP−) and fecal bacteria. These solutions include 100% fecal slurry (no added E. coli), 1 E. coli to 100 fecal bacteria, 1 E. coli to 10 fecal bacteria, 1 E. coli to 1 fecal bacterium, 10 E. coli to 1 fecal bacteria, 100 E. coli to 1 fecal bacterium, and 100% E. coli (no added fecal slurry). By estimating the number of bacteria in stool and the number of E. coli in our typical working solution, and varying the volumes of the fecal slurry and E. coli, we hold the total microbial load constant across each these seven solutions. The combined bacterial load between the fecal slurry and the E. coli is identical in each case.

For reversible inhibitors, to this bacterial inoculum is added profluorogenic substrate (100 μM final concentration), inhibitor (at a range of concentrations, for example, 0-400 μM), and PBS to a balance of 200 μL reaction volume. The contents of each well are mixed thoroughly. The plate is sealed with a lid and the fluorescence is recorded every 10 min for 24 h. For irreversible inhibitors, to each bacterial inoculum is added the test inhibitor (at a range of concentrations, for example, 0-400 μM), and the mixture is incubated at 37° C. for 30 min. The fluorogenic substrate is added (100 μM final concentration), and the fluorescence is recorded every 10 min for 24 h. At predefined timepoints, aliquots from the reaction are withdrawn and analyzed by LCMS to confirm the liberation of the fluorophore via an orthogonal method.

### Example 282: Using ClbP activity as a proxy for colibactin activity in a sample

A sample of colorectal cancer and/or healthy adjacent normal tissue is homogenized via physical means (for example, grinding on dry ice or with a handheld sonicator). The tissue is then resuspended in PBS pH 7.4, and a profluorogenic substrate is added to a concentration of 100 μM. The sample is incubated at 37° C. for 2 h and then centrifuged. The fluorescence is recorded as a proxy for the amount of colibactin that is produced.

Alternatively, after homogenized, the nucleic acids (either RNA or DNA) are extracted. For RNA, the total RNA is reverse transcribed into cDNA and then analyzed by qRT-PCR with primers specific to a gene required for colibactin biosynthesis, for example, ClbP. For DNA, the total DNA is extracted and then analyzed by qPCR with primers specific to a gene required for colibactin biosynthesis, for example, ClbP. The DNA can additionally be analyzed by whole genome or whole exome sequencing to look for mutations that are specifically reported to be induced by colibactin production.

### Example 283: Raising an antibody to the colibactin-DNA adduct

The colibactin-adenine adduct is generated in an in vitro assay by incubating a specific precolibactin with calf thymus DNA at 37° C. for 20 h. The reaction mixture is then heated at 100° C. for 1 h to liberate single nucleotides, lyophilized, and resuspended in acetonitrile/water. The colibactin-adenine adduct is isolated by using semi-preparative HPLC and lyophilized to dryness. The process is repeated to enable sufficient quantities of the molecule to be used in downstream purposes.

The adduct is used as an antigen to raise antibodies in mice or rabbits. The adduct is conjugated to a suitable non-immunogenic carrier protein and then is introduced into the animals bloodstream. The animals are immunized using an appropriate schedule. Optionally, this process may be made more effective by administration of an adjuvant. The animals sera is then screened for antibodies binding to the antigen, and the antibody titer and subtype are determined. If additional characterization is desirable, the antibody is purified by using its ability to bind to the colibactin-adenine adduct. The antibody can be immobilized for usage in ELISA protocols and as a way to detect or quantify the amount of colibactin-DNA adduct present in cells.

### Example 284: In vitro cell coinfection assay

E. coli Nissle 1917 and E. coli BL21(DE3) are prepared as specified in the section “Growth and concentration of bacterial strains.” The bacterial cell count for each is determined using a Quantom cell counter protocol. The bacteria are centrifuged, washed with 1×Dulbecco's phosphate-buffered saline (DPBS), centrifuged, and resuspended in 1×DPBS. Separately, HeLa cells are grown to confluence in DMEM medium. Each bacterial strain is then incubated with the HeLa cells at multiplicity of infection (MOI) of 1000:1, 100:1, or 10:1 bacterial cells to HeLa cells for 1 h at 37° C. The HeLa cells are washed with 1×DPBS to remove E. coli and then incubated in gentamicin for 23 h at 37° C. to kill any residual E. coli. The HeLa cells are isolated and analyzed by an LCMS method to quantify the amount of colibactin-adenine adduct that is formed at each MOI. Alternatively, after the E. coli is removed with washing and gentamicin treatment, the HeLa cells can be isolated and examined for general DNA damage using a Western blot to look for γH2AX. Doing so would enable an orthogonal quantitative measurement of the amount of colibactin produced through the pathway, particularly in comparison to the E. coli ΔcIbP S95A strain. As a third orthogonal method for quantifying damage due to colibactin production, after the E. coli is removed, the whole genomic DNA from the HeLa cells is extracted and analyzed by whole genome sequencing. A computational method is used to quantify the number of mutations (for example, single nucleotide polymorphisms or indels) that have accumulated at adenine-thymidine rich stretches of DNA that are reported to be damaged specifically by colibactin. This measurement provides a readout at the extent of colibactin production and its reduction in the presence of an inhibitor.

To test for the ability of inhibitors to suppress formation of the colibactin-adenine adduct specifically or DNA damage in general, the above protocol is used and the inhibitors are spiked into the coinfection media with the E. coli. The inhibitors are amended to the reaction mixture across a range of concentrations (for example, 0-400 μM). The bacteria is removed as above, and the colibactin-adenine adduct is quantified by LCMS. Doing so will provide a dose-response curve for the amount of colibactin-adenine adduct as a function of inhibitor concentration and provide an IC50 for the coinfection assay. Alternatively, a Western blot for γH2AX and densitometry is performed to obtain a quantitative measurement of the amount of DNA damage occurring due to colibactin and its possible reduction by small molecule inhibitors. Optionally, the whole genomic DNA as above and a computational method is used to assess the amount of DNA at colibactin-specific stretches of DNA.

### Example 285: Antibiotic assessment

E. coli Nissle 1917 and E. coli BL21(DE3) are prepared as specified in the section “Growth and concentration of bacterial strains.” To assess if inhibitors are bactericidal, the inhibitors are incubated across a range of concentrations (for example, 0-400 μM) with the saturated cultures for 2 h at 37° C., and then a dilution series is plated onto BHI plates. The plates are grown for 24 h at 37° C., and then plates with between 30 and 300 colonies are assessed for how many colonies they each contain, with a bacterialcidal clearly showing a reduction in the colony forming units compared to a vehicle control.

To assess if the inhibitors are bacteriostatic or bactericidal, E. coli Nissle 1917 and E. coli BL21(DE3) are prepared as specified in the section “Growth and concentration of bacterial strains.” The saturated cultures are used to freshly inoculate fresh LB media at a ratio of 1:100. The strains are then grown in a plate reader at 37° C., and the OD600 is recorded every 10 min. At mid-log growth (an OD600 of 0.4, for example), inhibitor is added across a range of concentrations (for example, 0-400 μM). The cultures are incubated at 37° C., and the OD600 is then recorded every 10 min for 6 hours. Bacteriostatic compounds result in premature growth termination. Bactericidal compounds result in a reduction in the OD600.

### Example 286: Time-dependent inhibition of the enzyme for irreversible inhibitors

An aliquot of WT ClbP or S95A ClbP is thawed on ice. The compounds are added to each protein (1 μM) across a range of concentrations (for example, 0, 5, 10, 20, 40, 100, 200, and 400 μM final concentration) in assay buffer (50 mM sodium phosphate, 200 mM NaCl, 0.02% (w/v) DDM, pH 8.0). The solution is incubated at 37° C. At different times of preincubation (for example, 0, 5, 10, 15, 30, 45, 60, and 90 min after combining the protein and the inhibitor), a portion of protein is withdrawn from each of the different inhibitor concentrations. The residual activity of this protein is immediately assessed by diluting it 10 to 100-fold into assay buffer containing a profluorogenic substrate (100 μM final concentration) and recording the fluorescence in a BioTek Synergy HTX Multimode plate reader. The plate is read from the bottom or top with an excitation wavelength of 360 nm (40 nm bandwidth) and the emission wavelength is 460 nm (40 nm bandwidth). The light source is tungsten, the gain is 35, and the speed read is normal.

This experiment provides a time-dependent inactivation curve for each tested inhibitor concentration. Each curve is fit to a single exponential decay to extract the rate constant (k_{obs}) at each inhibitor concentration, and then the k_{obs} vs inhibitor concentration is fit to a hyperbola to derive the parameters k_{inact} and K₁. These are the rate constants for covalent, irreversible inhibitors.

### Example 287: Inhibitor binding evaluated by thermal denaturation

An aliquot of WT ClbP or S95A ClbP is thawed on ice. In a RT-PCR compatible 96-well plate, the compounds are added to each protein (1 μM final concentration) across a range of concentrations (for example, 0, 5, 10, 20, 40, 100, 200, and 400 μM final concentration) in assay buffer (50 mM sodium phosphate, 200 mM NaCl, 0.02% (w/v) DDM, pH 8.0) with SYPRO Orange (2×, from a stock solution of SYPRO Orange 5000×). The contents of each well are mixed thoroughly by pipetting, and then the plate is placed into an instrument capable of performing RT-PCR, for example a QuantStudio 6. A thermal denaturation curve is run using the following protocol:

### 1) Incubate at 22° C. for 1 min.

2) Record the fluorescence (excitation wavelength of 470 nm, emission wavelength of 570 nm).
3) Increase the temperature by 1° C. and incubate for 30 s.
4) Record the fluorescence (excitation wavelength of 470 nm, emission wavelength of 570 nm).
5) Go to step 3 73 times such that the final temperature is 95° C.
6) End the protocol.

The temperature-dependent fluorescence at each concentration of potential inhibitor can then be fit using known methodology to extract a melting temperature shift. Thermal stabilization at increasing concentrations of inhibitors indicates thermal stabilization and a likely binding event.

### Example 288: Counterscreen against AmpC

AmpC protein (UniProt ID: P00811) is expressed and purified as previously reported. The AmpC protein (10 nM) is incubated with its substrate CENTA (60 μM), and the product formation at 405 nm is monitored via the released chromophore as previously reported. The AmpC activity is modulated by amending the reaction mixture to contain authentic AmpC inhibitors, recapitulating the reported inhibitory activity. The AmpC/CENTA reaction mixture is also amended to contain test inhibitors developed against ClbP, and the IC50 of these ClbP inhibitors is determined to evaluate their ability to selectively inhibit ClbP and not closely-related enzymes present in the GI microbiome.

### Example 289: Mouse GI time course/plasma PK

C57BL/6J mice aged 6-8 weeks are gavaged with a therapeutic dose of inhibitor. At 0, 30, 60, 240, and 1440 minutes post-gavage, blood is drawn from a group of 3 mice and the mice are sacrificed. The intestinal tract is harvested. The amount of inhibitor present in circulation and in the colon at each timepoint is determined by LCMS quantification against an isotopically labeled inhibitor or internal standard. Optionally, the products of potential microbial or hepatic metabolism may be determined by LCMS.

### Example 290: Germ-free mouse model for disruption of colibactin adducts

Germ-free C57BL/6J mice aged 6-8 weeks are housed in two separate incubators. On day 0, the animals are gavaged with either 5×10⁷ CFUs E. coli Nissle 1917 (first incubator) or E. coli Nissle 1917 ΔcIbP in a volume of 100 μL PBS (second incubator). At days 7 and day 14, fecal pellets from each incubator are harvested. On day 14, the mice are sacrificed, and colons are harvested from each mouse. The intestinal colonic epithelial cells are isolated by dissociating the tissue by rocking in PBS containing EDTA, dithiothreitol, and FBS for 10 min on ice. The tissues are vortexed and passed through a 100 μm strainer to collect the epithelial cells. The colibactin-adenine adduct is quantified by LCMS as above. Optionally, a third group of germ-free C57BL/6J mice is also included and gavaged with 5×10⁷ CFUs E. coli Nissle 1917 in a volume of 100 μL PBS. This group is also be treated gavaged once per day with a therapeutic dose of inhibitor. After 14 days colonic epithelial cells are harvested and the colibactin-adenine adduct is quantified to determine the efficacy of the tested inhibitor. Alternatively, the intestinal epithelial cells are isolated and examined for general DNA damage using a Western blot to look for γH2AX. Doing so would enable an orthogonal quantitative measurement of the amount of colibactin produced through the pathway, particularly in comparison to the E. coli ΔcIbP S95A strain. As a third orthogonal method for quantifying damage due to colibactin production, whole genome sequencing is performed on the isolated epithelial cells. A computational method is used to quantify the number of mutations (for example, single nucleotide polymorphisms or indels) that have accumulated at adenine-thymidine rich stretches of DNA that are reported to be damaged specifically by colibactin. This measurement provides a readout at the extent of colibactin production and its reduction in the presence of an inhibitor.

### Example 291: Animal disease model for evaluating carcinogenesis due to colibactin production

Mucosal inflammation is induced in male APC^{Min/+} mice by exposing them to 2% (w/v) in their drinking water from days 0-7. One group is a naive control to monitor disease progression, a second group housed separately is gavaged with 5×10⁷ CFUs E. coli Nissle 1917 in 100 μL PBS, and a third group housed separately is gavaged with 5×10⁷ CFUs E. coli Nissle 1917 ΔcIbP S95A in 100 μL PBS, and a fourth group housed separately is gavaged with 5×10⁷ CFUs E. coli Nissle 1917 in 100 μL PBS as well as an inhibitor at a therapeutic dose. Animals are monitored daily for survival, the presence or absence of diarrhea, and the presence or absence of blood in the stool. Colitis, polyp size, and the number of polyps are assessed by video endoscopy on days 7, 14, 21, and 28. Surviving animals are euthanized on day 28. Blood (plasma) and colon (the most distal 5 cm: proximal and distal 2 cm formalin-fixed and middle 1 cm flash-frozen) are collected at euthanasia.

The reduction in carcinogenesis from colibactin production is assessed by quantifying the colitis severity using a clinical score, the polyp size, and the number of polyps. Furthermore, the colonic epithelial cells are harvested, and can be assessed for the quantity of colibactin-adenine adduct as determined by LCMS, the amount of general DNA damage by performing a Western-blot for γH2AX, or the amount of mutations accumulating at colibactin-specific stretches of DNA by whole genome sequencing (as in the section “Germ-free mouse model for disruption of colibactin adducts”). Reduction in these measurements as well as gross biology (colitis severity, polyp size, polyp number) confirm that the inhibitors can decrease colibactin production and carcinogenesis in a translatable disease model.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A compound of Formula A): 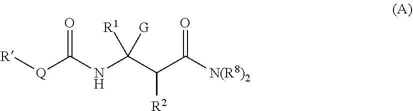
a pharmaceutically acceptable salt thereof, or a solvate of any of the foregoing,
R′ is chosen from hydrogen, C₁₋₁₄ alkyl and C₁₋₁₄ alkenyl, wherein alkyl and alkenyl are optionally substituted;
Q is CR″R″′, NR″, or O;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl; and
G is chosen from C(═O)CF₃, C(═O)H, B(OH)₂, B(Oalkyl)₂, CN, P(═O)(Oaryl)₂, P(═O)(OCH₃)F, S(═O)₂F, C(═O)aryl, C(═O)heteroaryl, 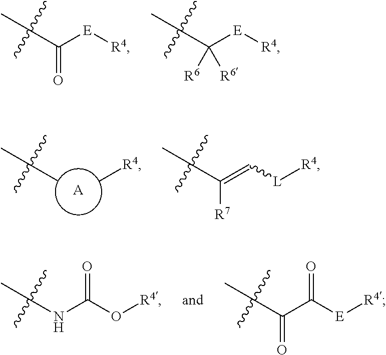
Wherein alkyl, aryl, and heteroaryl are optionally substituted;
wherein:
E is chosen from CR³R³′, NR³, and O, wherein optionally R¹ and R³ or R² and R³ together form a ring;
L is chosen from S(═O)₂, C(═O), C(═O)NR³, and a bond;
R³ and R^{3′} each independently is chosen from H, halogen, and C₁₋₃ alkyl;
Ring A is chosen from 4-8 membered heteroaryl and 4-8 membered heterocyclyl; wherein the 4-8 membered heteroaryl or 4-8 membered heterocyclyl is optionally substituted;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted;
R^{4′} is chosen from C₁₋₇ alkyl and C₀₋₆ alkyl-R⁵; wherein the C₁₋₇ alkyl or C₀₋₆ alkyl-R⁵ is optionally substituted;
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted;
R⁶ and R^{6′} each independently is chosen from H, OH, haloalkyl, and C₁₋₃ alkyl, or R⁶ and R^{6′} together form a 4-6 membered ring; and
R⁷ is chosen from H, C₁₋₃ alkyl, and halogen.

2. The compound of claim 1, wherein R′ is heptyl.

3. The compound of claim 1 or 2, wherein Q is NH.

4. The compound of claim 1 or 2, wherein Q is CH₂.

5. A compound of claim 1 chosen from: 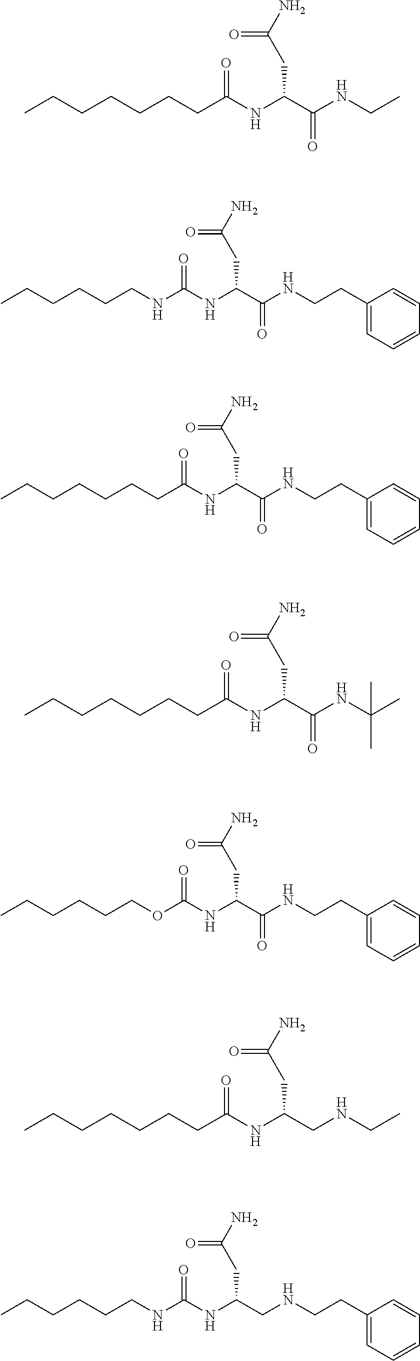 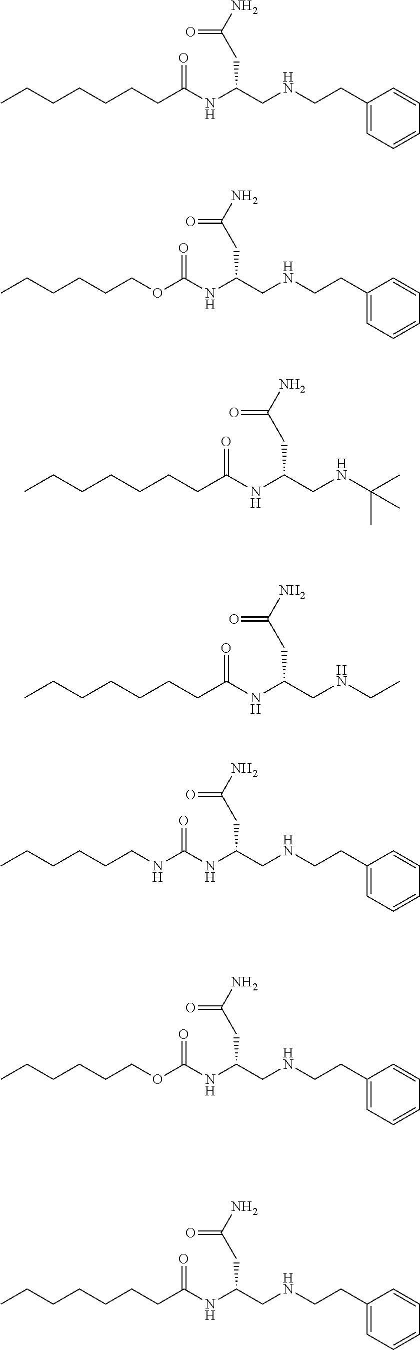 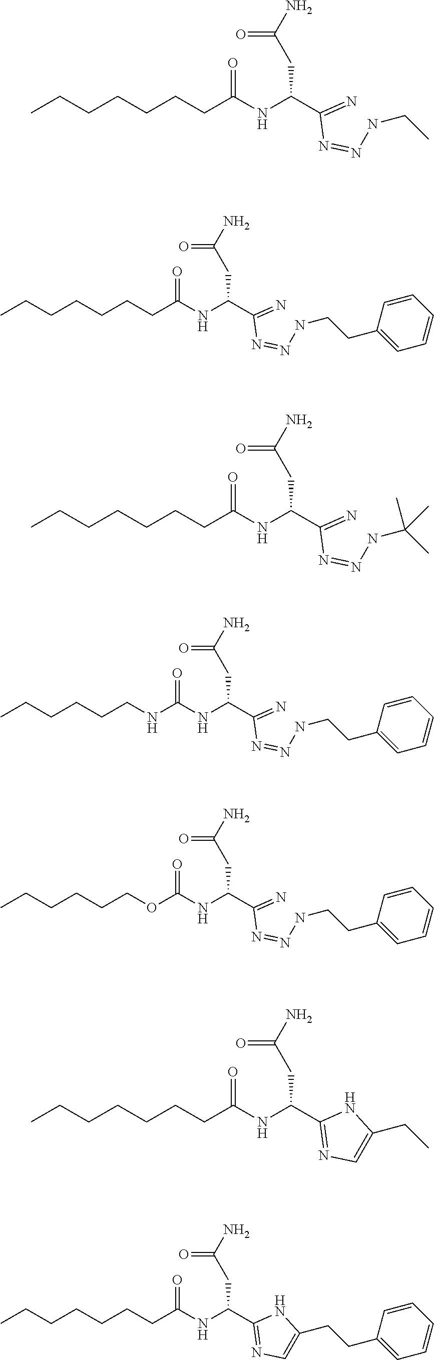 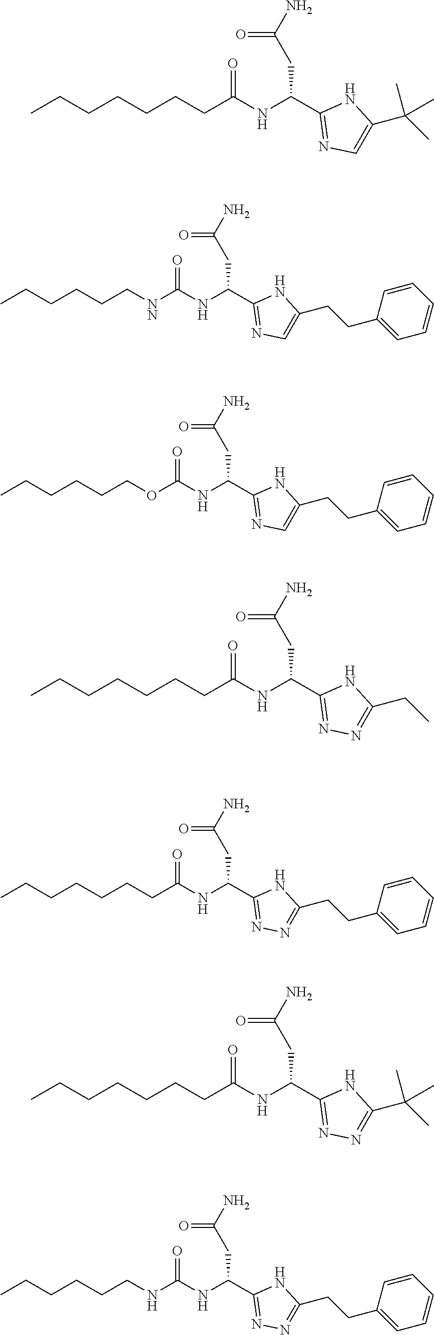 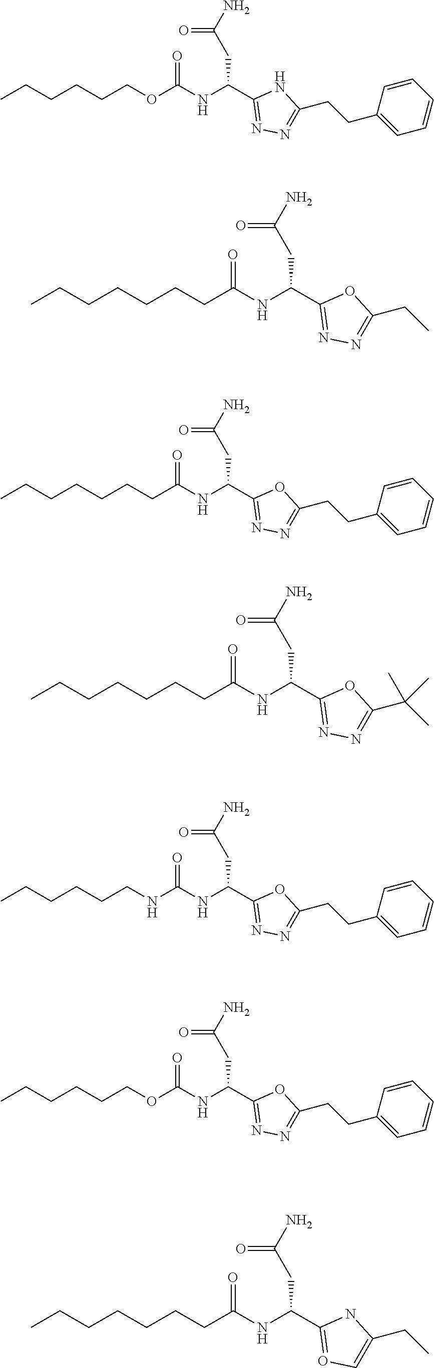 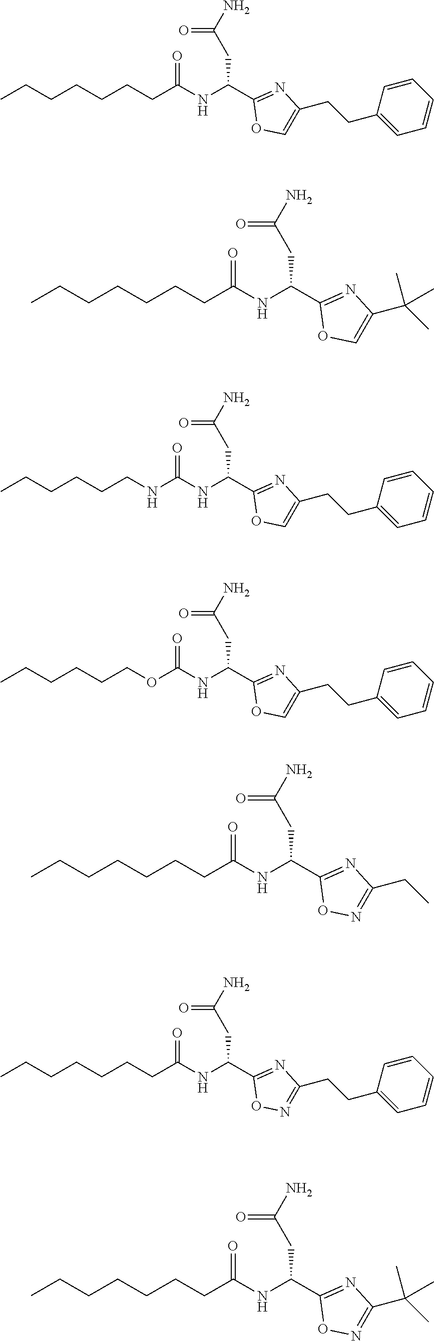 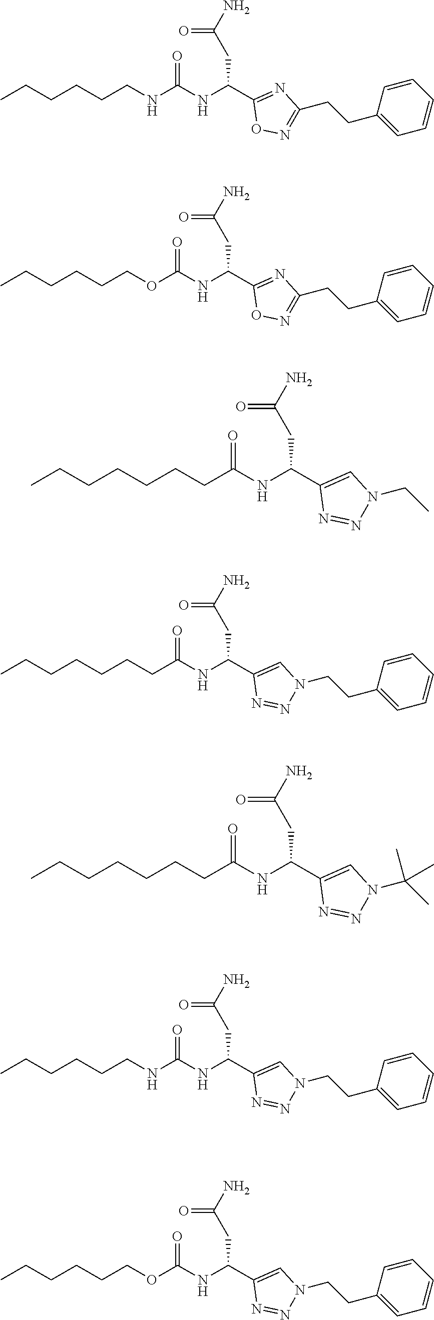 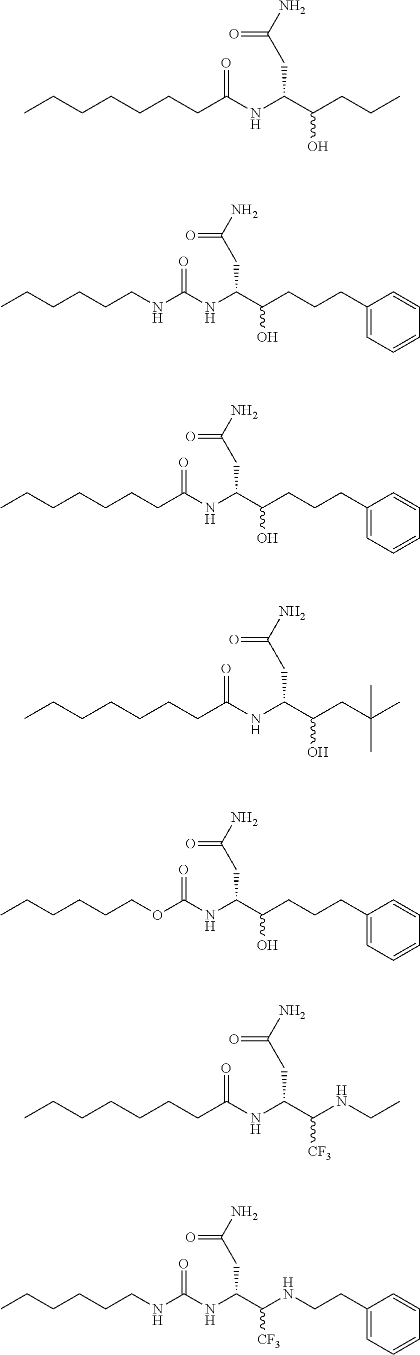 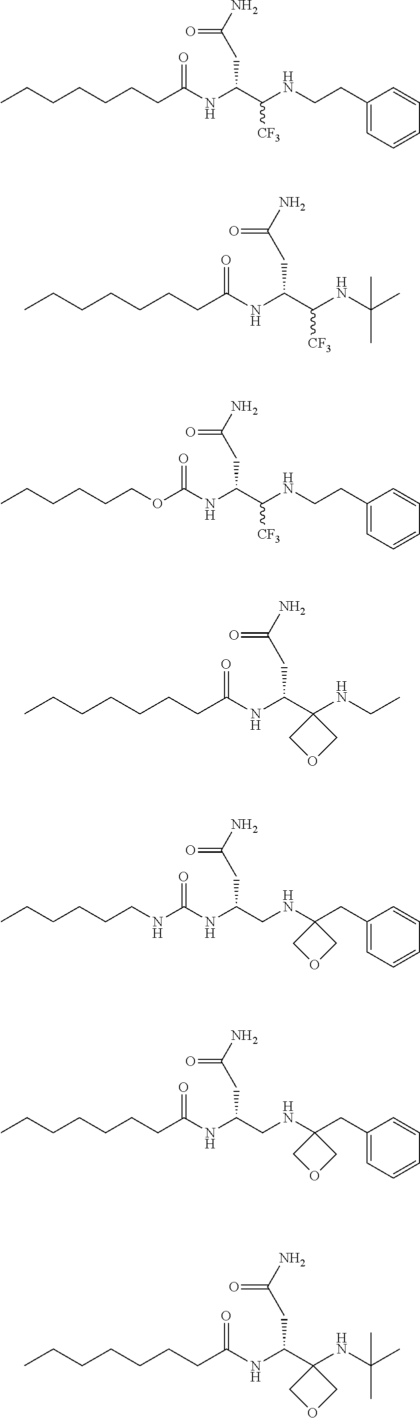 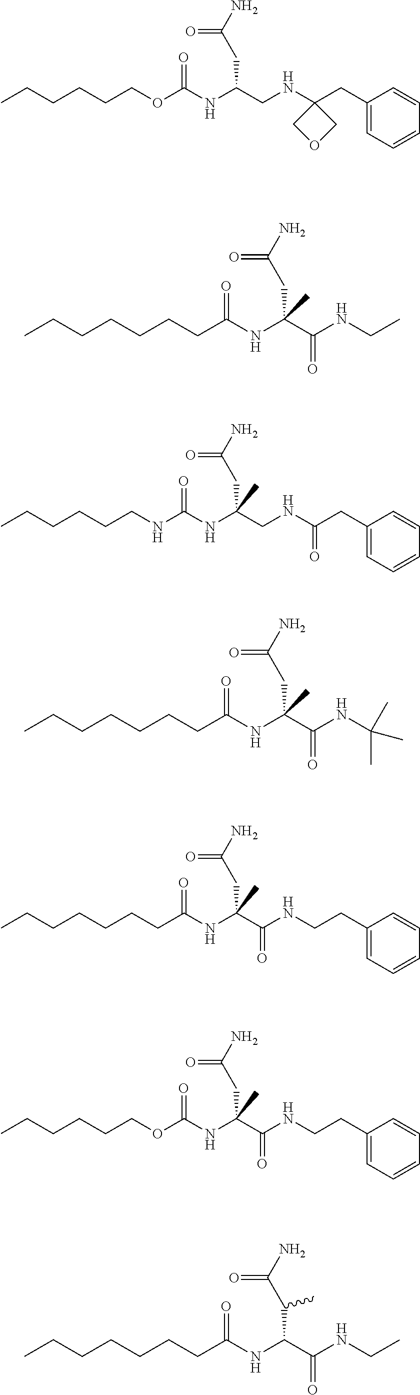 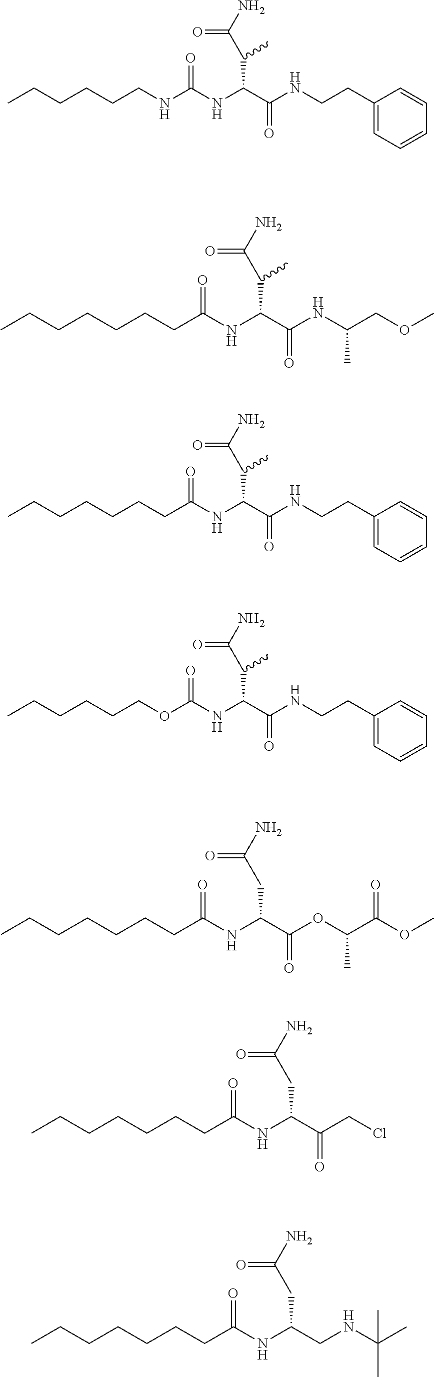 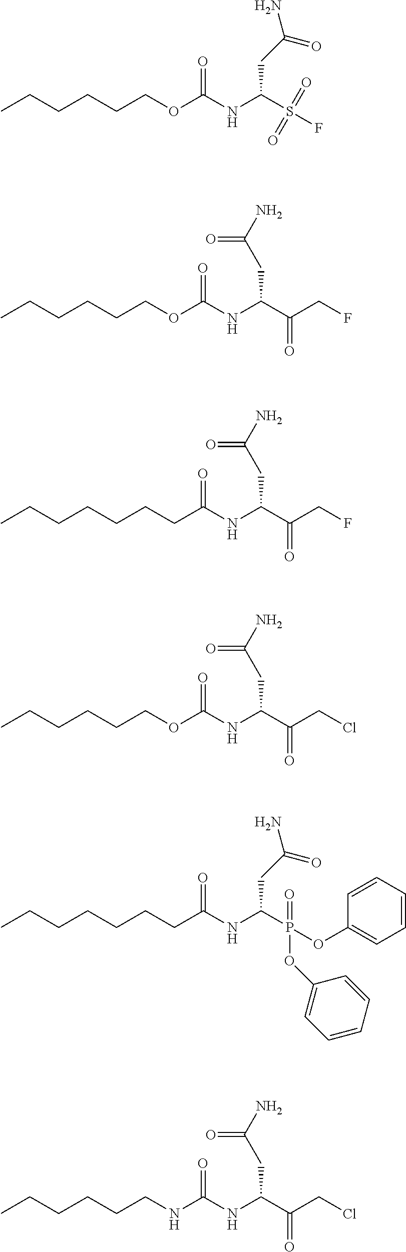 [SMILES: CCCCCCOC(=O)N[C@H](CC(N)=O)S(=O)(=O)F ; CCCCCCOC(=O)N[C@H](CC(N)=O)C(=O)CF ; CCCCCCCC(=O)N[C@H](CC(N)=O)C(=O)CF ; CCCCCCOC(=O)N[C@H](CC(N)=O)C(=O)CCl ; CCCCCCCC(=O)N[C@H](CC(N)=O)P(=O)(Oc1ccccc1)Oc1ccccc1 ; CCCCCCNC(=O)N[C@H](CC(N)=O)C(=O)CCl] 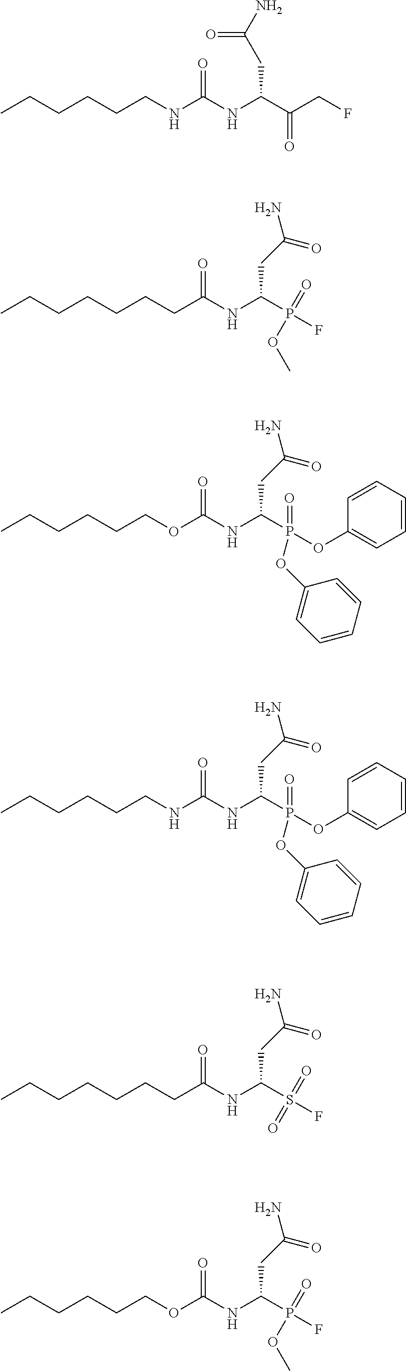 [SMILES: CCCCCCNC(=O)N[C@H](CC(N)=O)C(=O)CF ; CCCCCCCC(=O)N[C@H](CC(N)=O)P(=O)(F)OC ; CCCCCCOC(=O)N[C@H](CC(N)=O)P(=O)(Oc1ccccc1)Oc1ccccc1 ; CCCCCCNC(=O)N[C@H](CC(N)=O)P(=O)(Oc1ccccc1)Oc1ccccc1 ; CCCCCCCC(=O)N[C@H](CC(N)=O)S(=O)(=O)F ; CCCCCCOC(=O)N[C@H](CC(N)=O)P(=O)(F)OC] 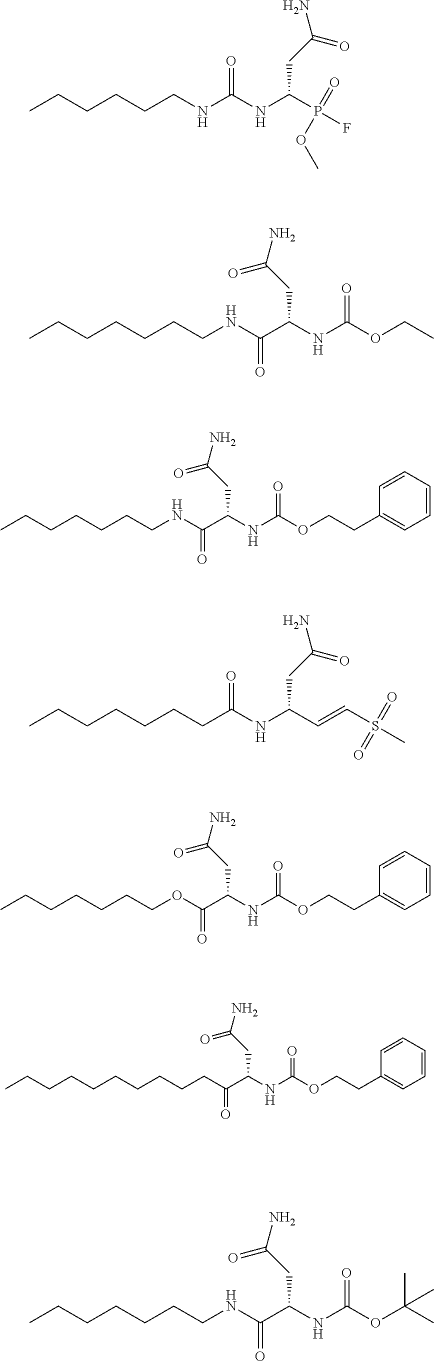 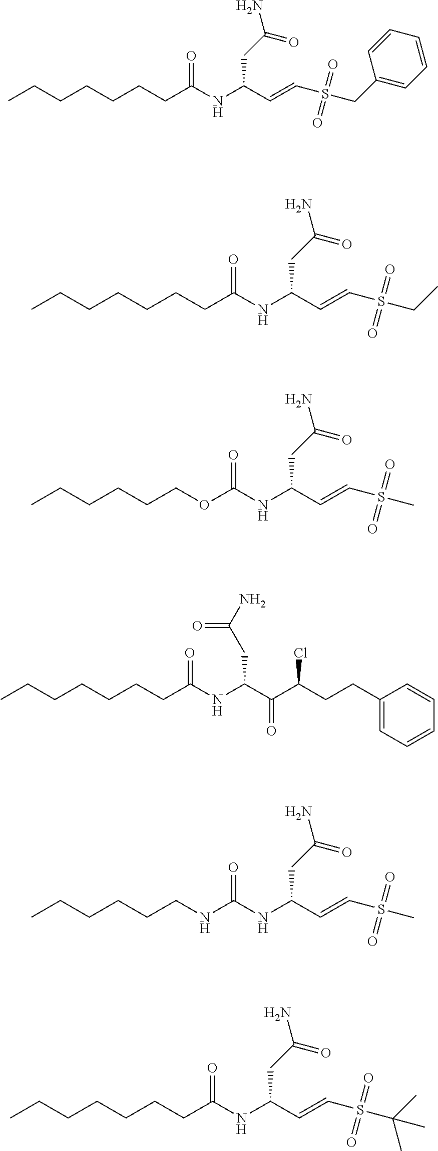 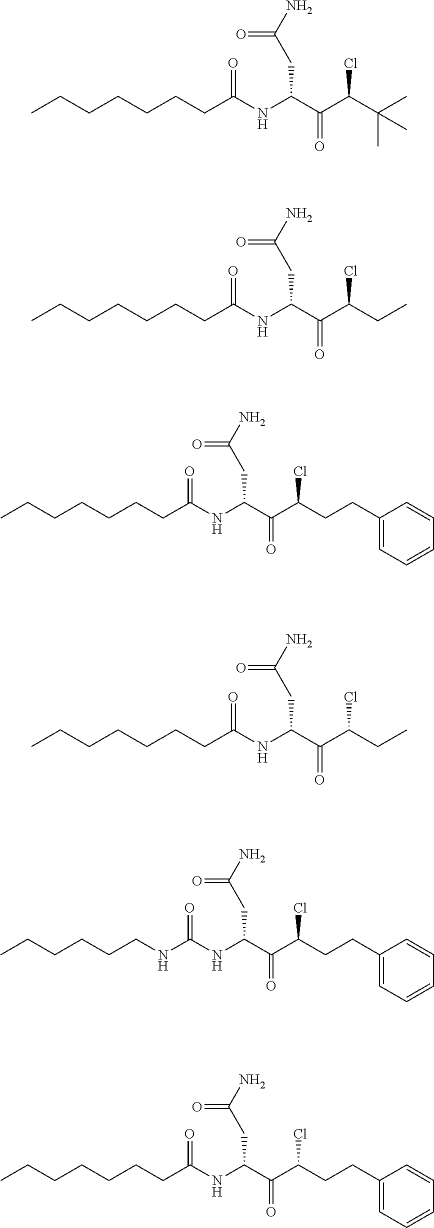 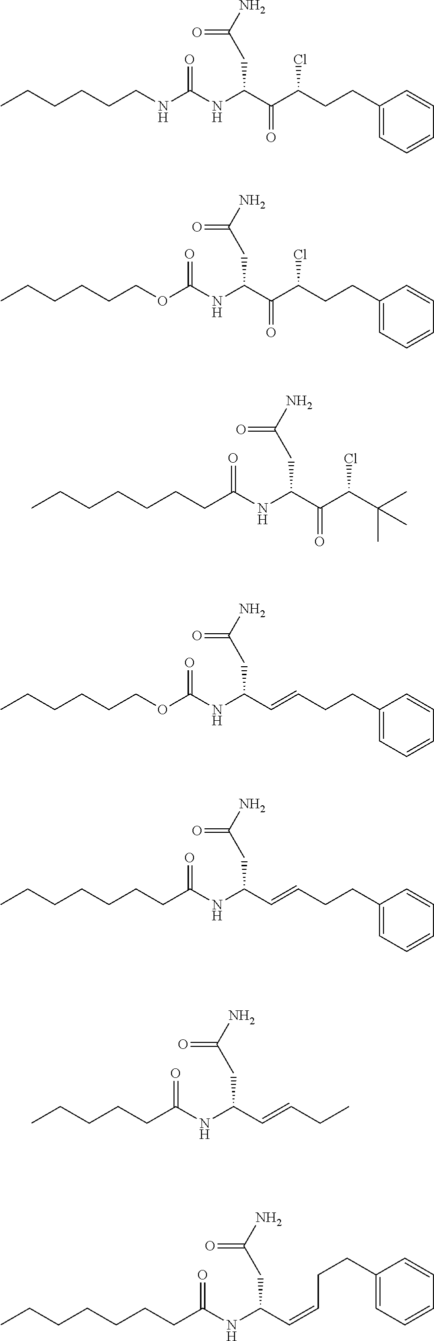 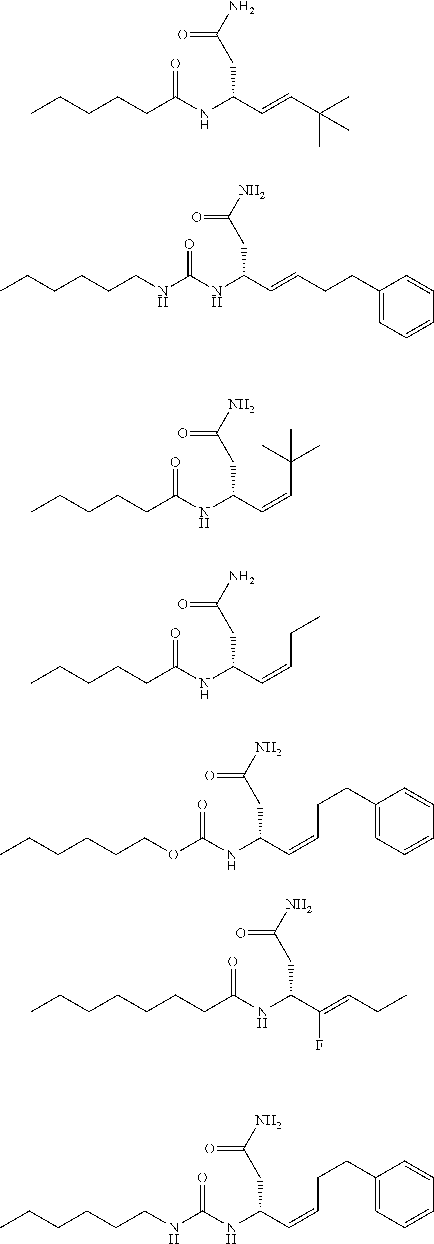 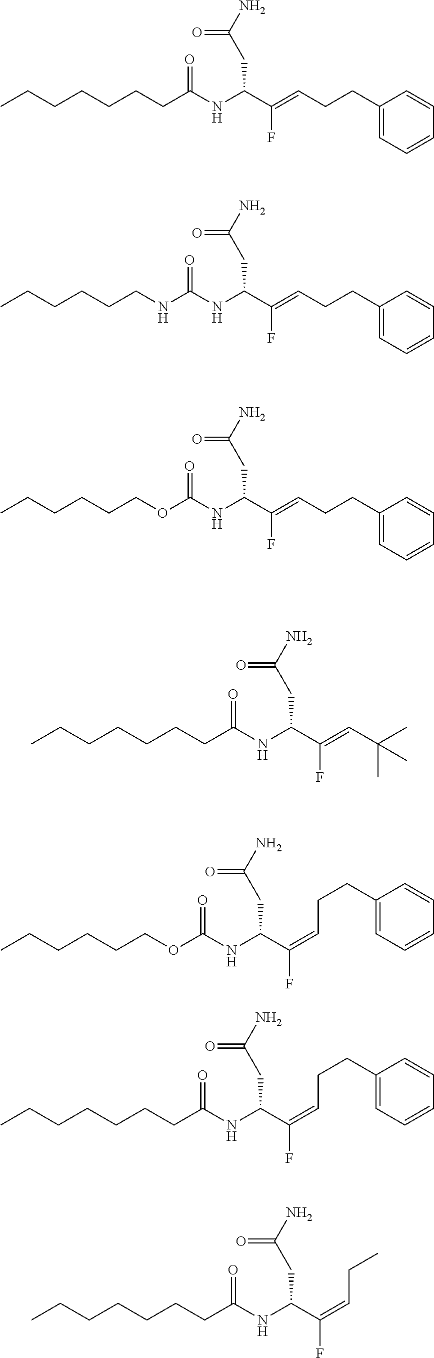 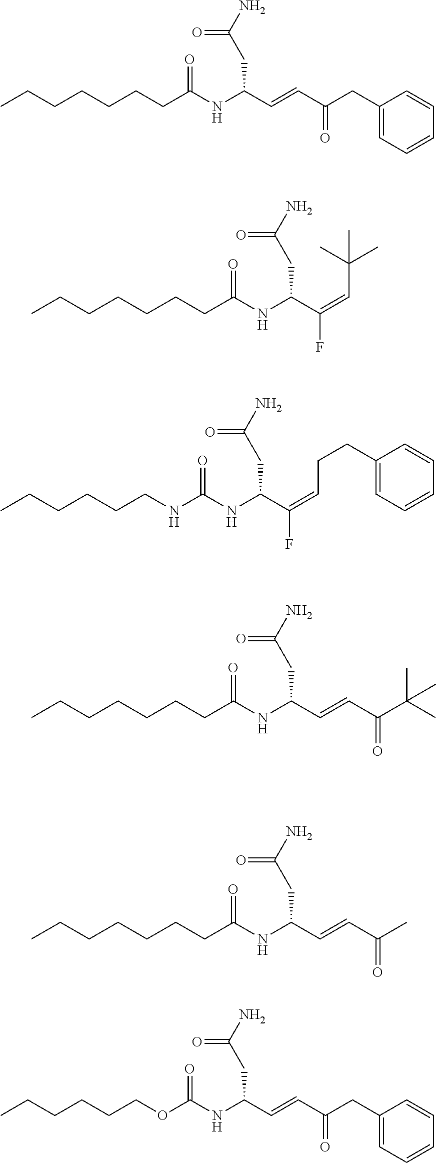 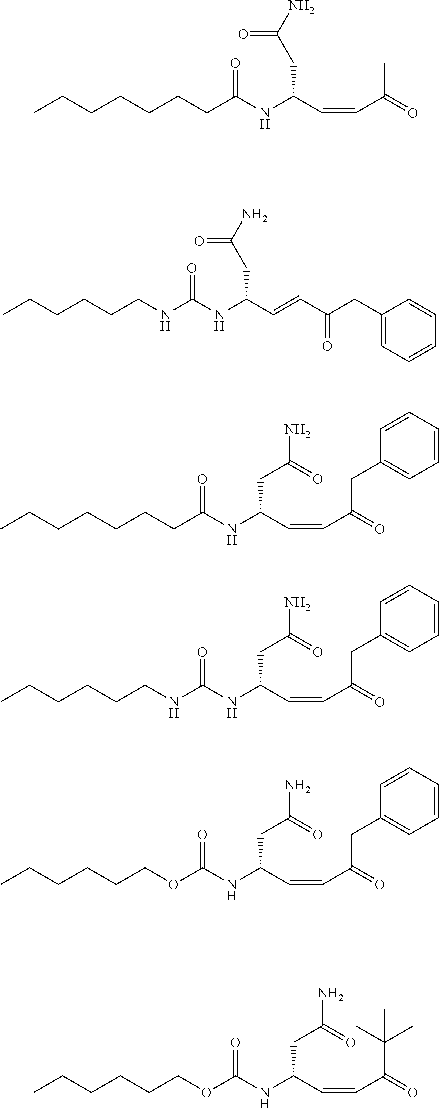 [SMILES: CCCCCCCC(=O)N[C@@H](/C=C\C(C)=O)CC(N)=O ; CCCCCCNC(=O)N[C@@H](/C=C/C(=O)Cc1ccccc1)CC(N)=O ; CCCCCCCC(=O)N[C@@H](/C=C\C(=O)Cc1ccccc1)CC(N)=O ; CCCCCCNC(=O)N[C@@H](/C=C\C(=O)Cc1ccccc1)CC(N)=O ; CCCCCCOC(=O)N[C@@H](/C=C\C(=O)Cc1ccccc1)CC(N)=O ; CCCCCCOC(=O)N[C@@H](/C=C\C(=O)C(C)(C)C)CC(N)=O] 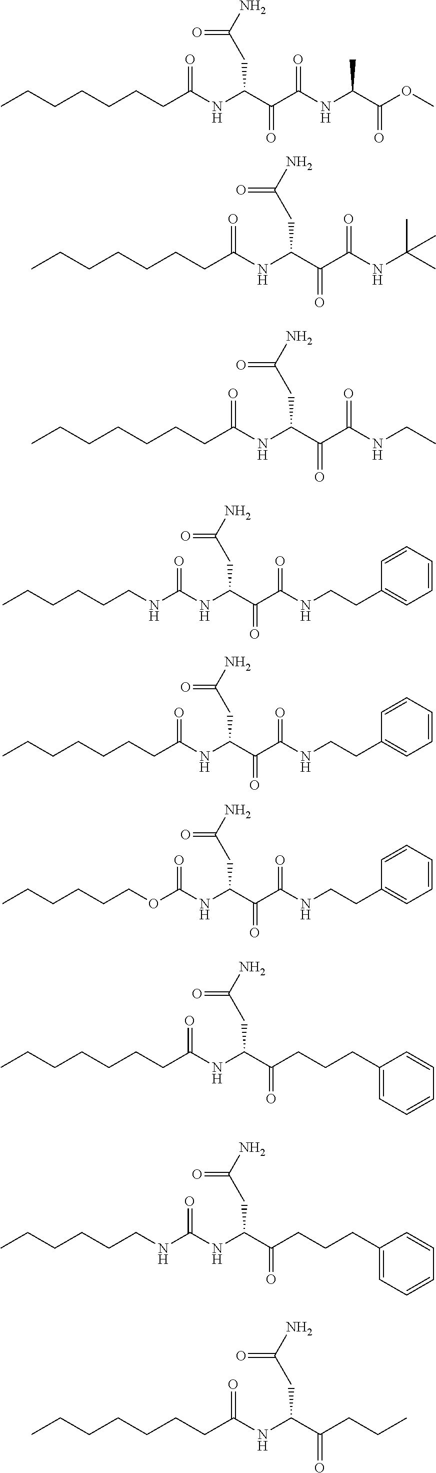 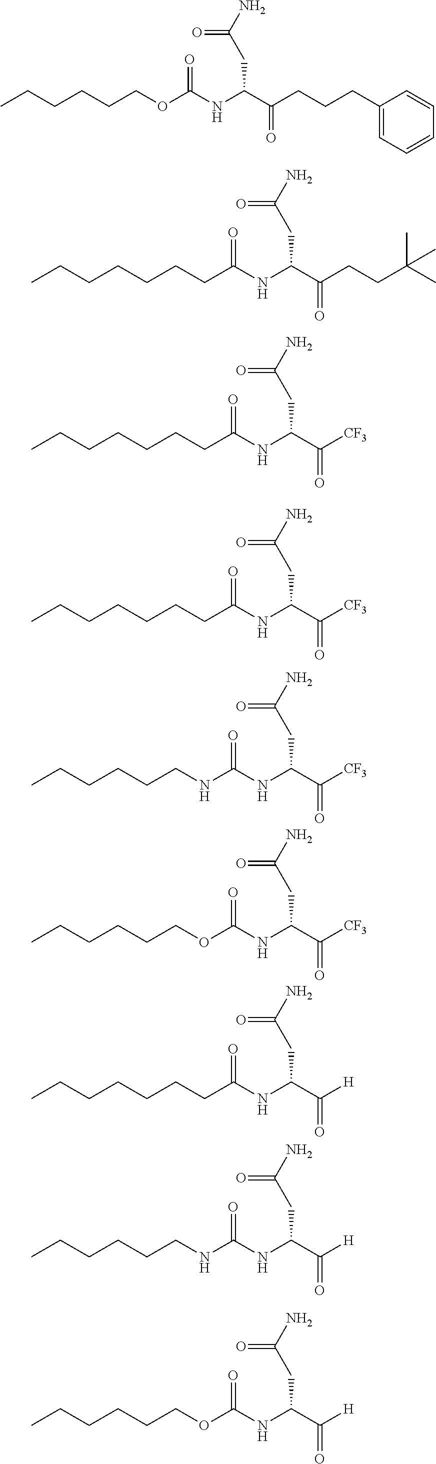 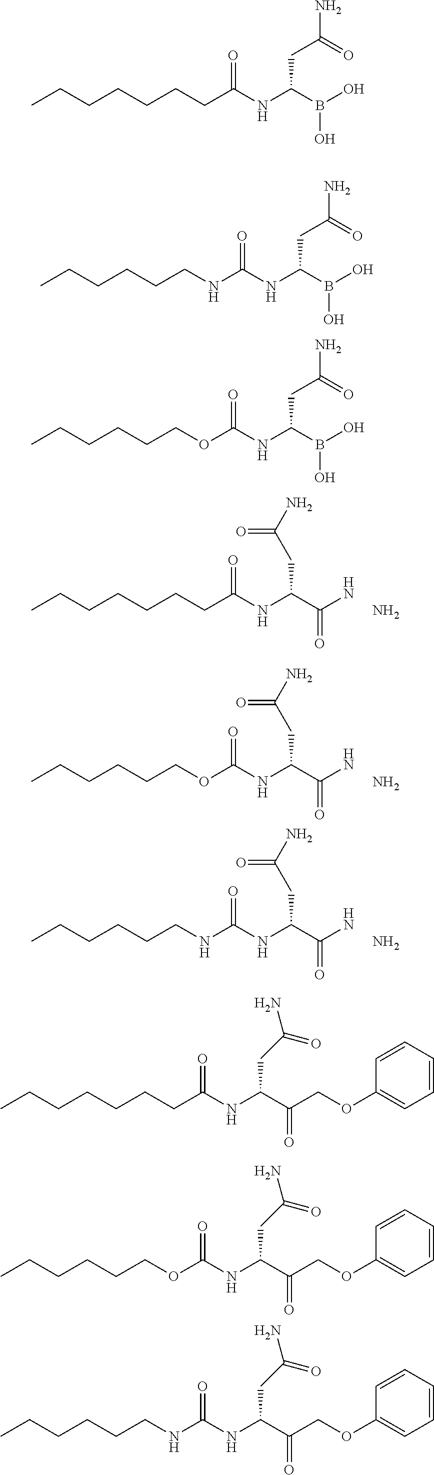 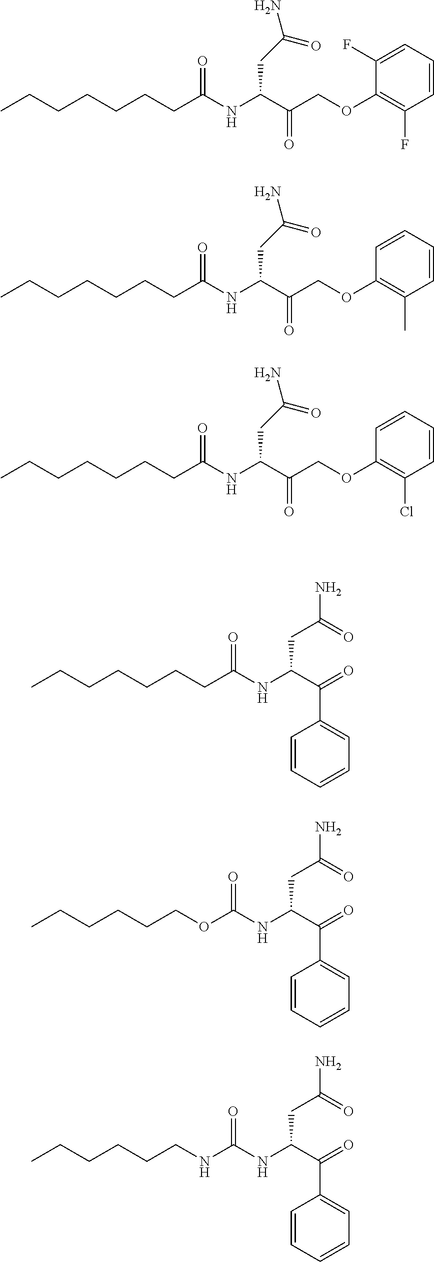 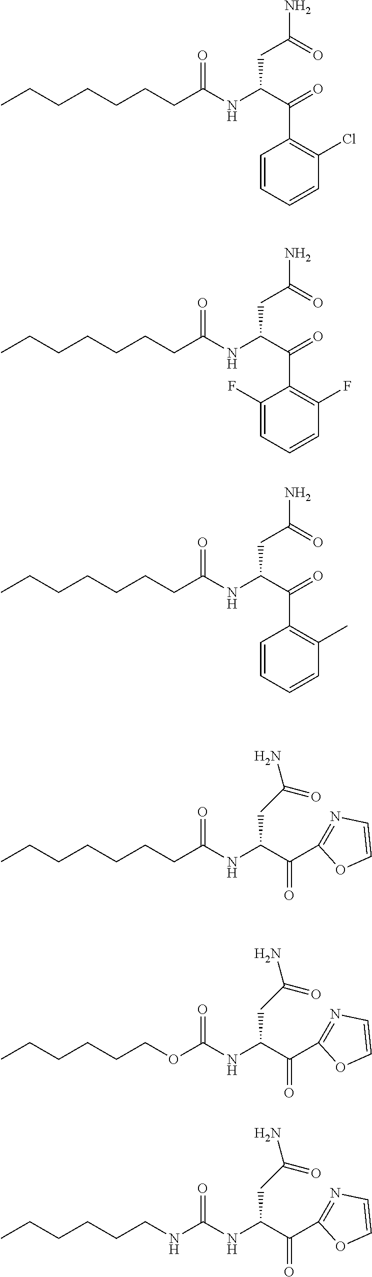 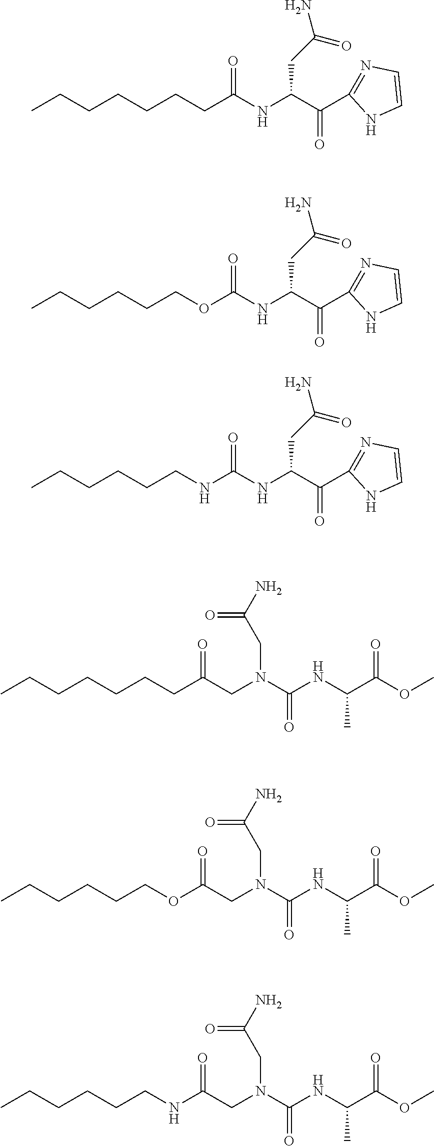 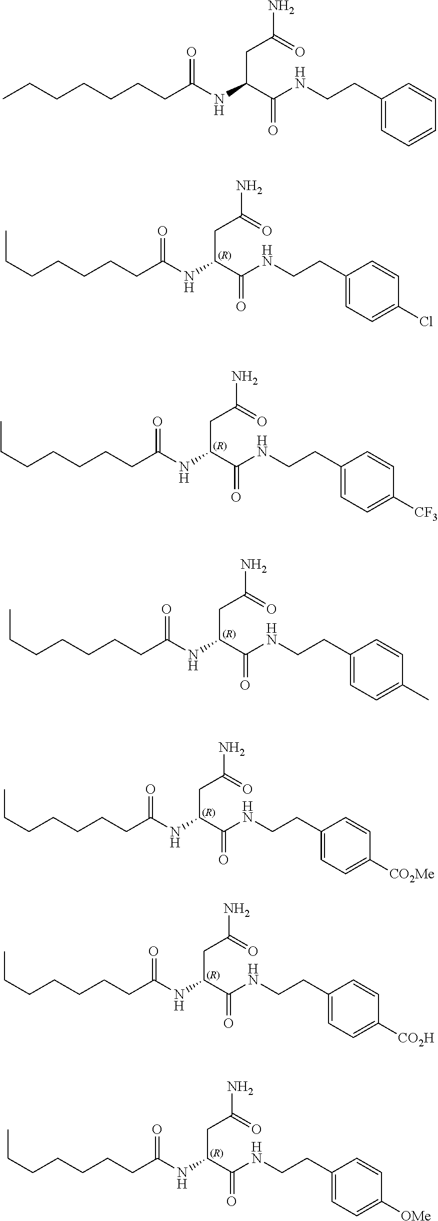 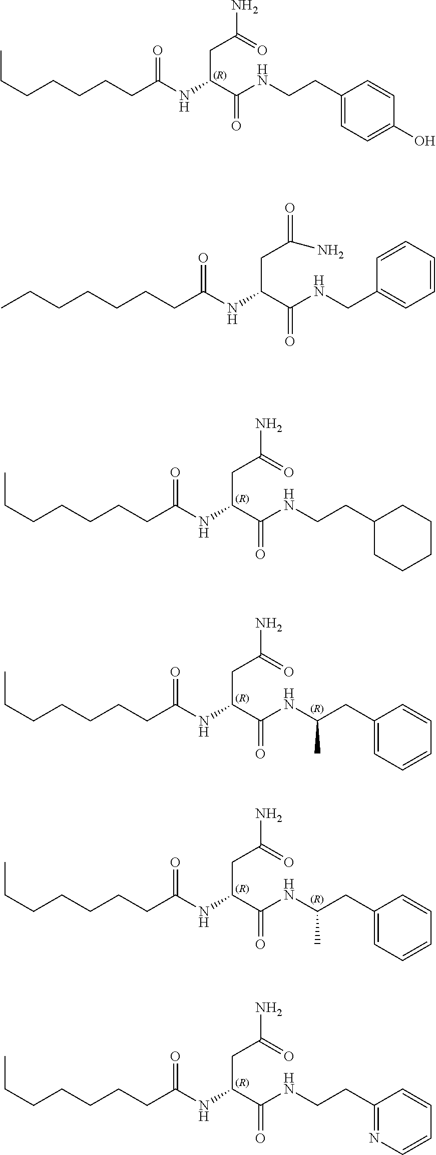 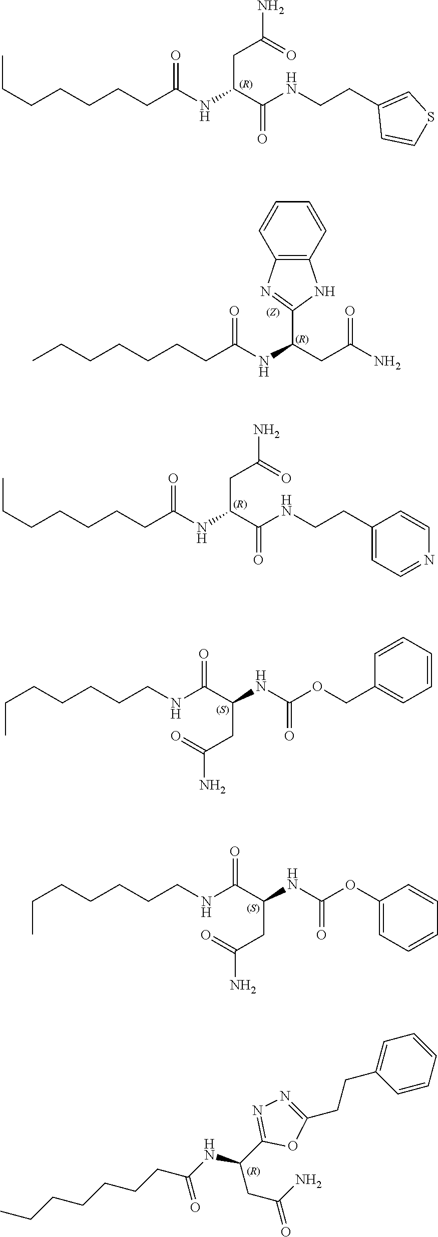 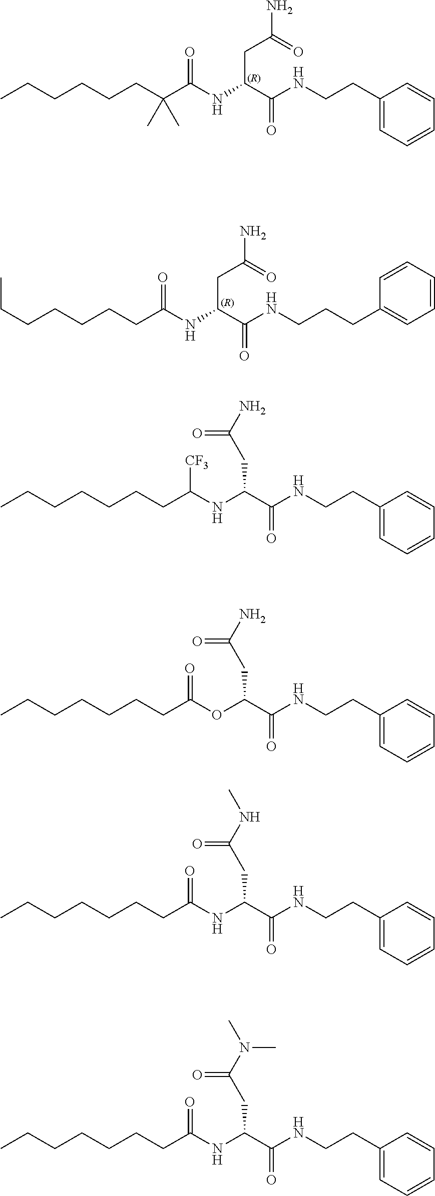 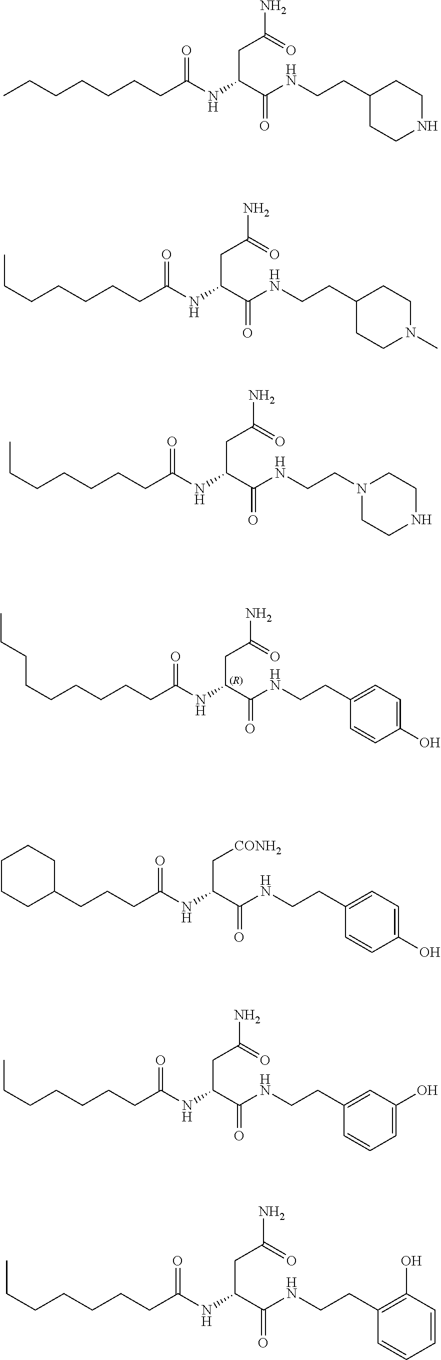 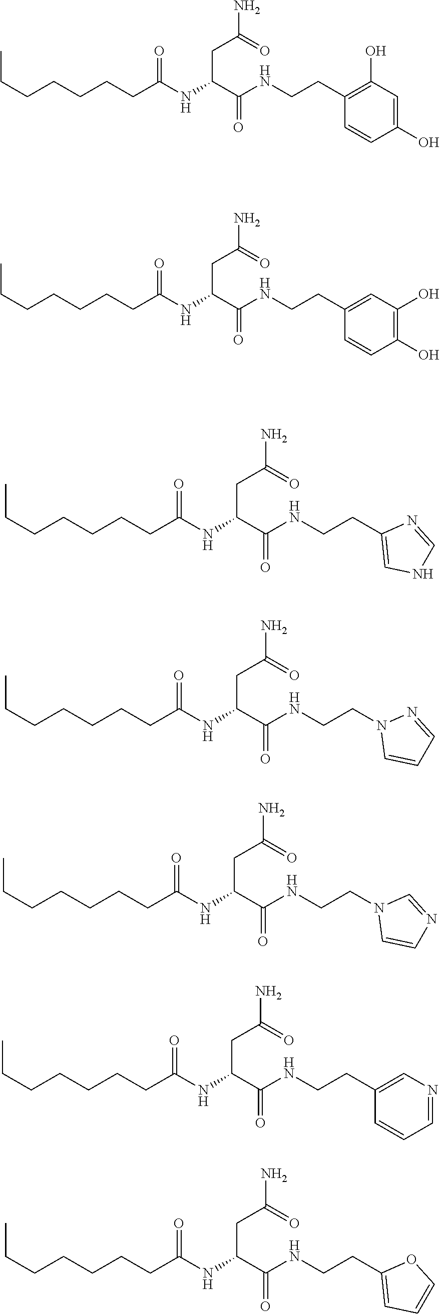 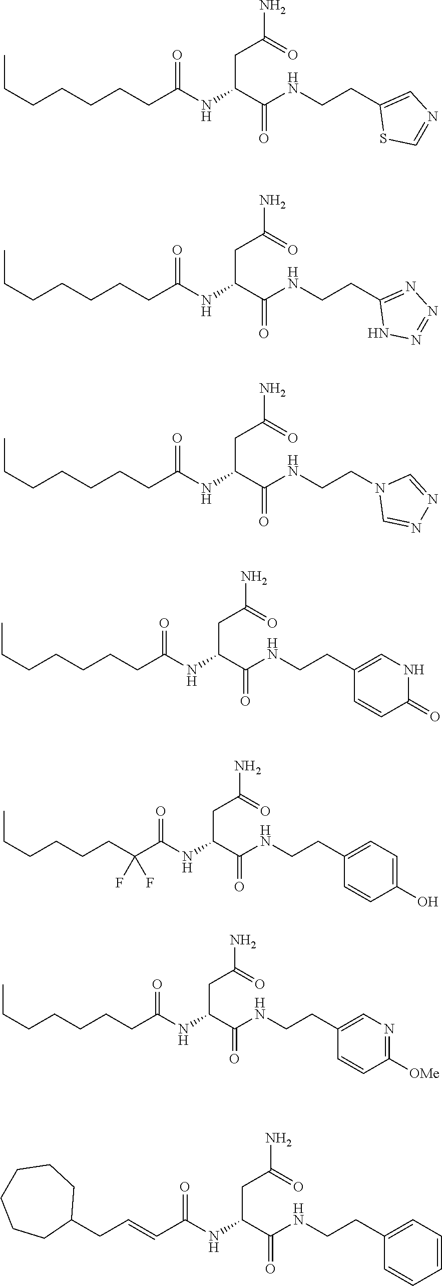 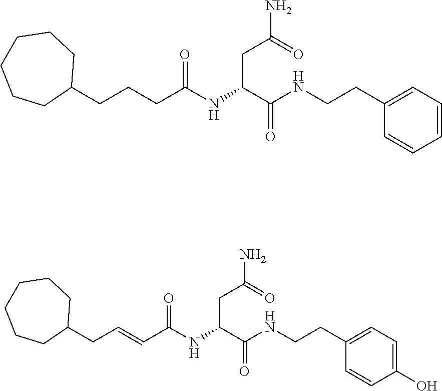 [SMILES: NC(=O)C[C@@H](NC(=O)CCCC1CCCCCC1)C(=O)NCCc1ccccc1 ; NC(=O)C[C@@H](NC(=O)/C=C/CC1CCCCCC1)C(=O)NCCc1ccc(O)cc1] 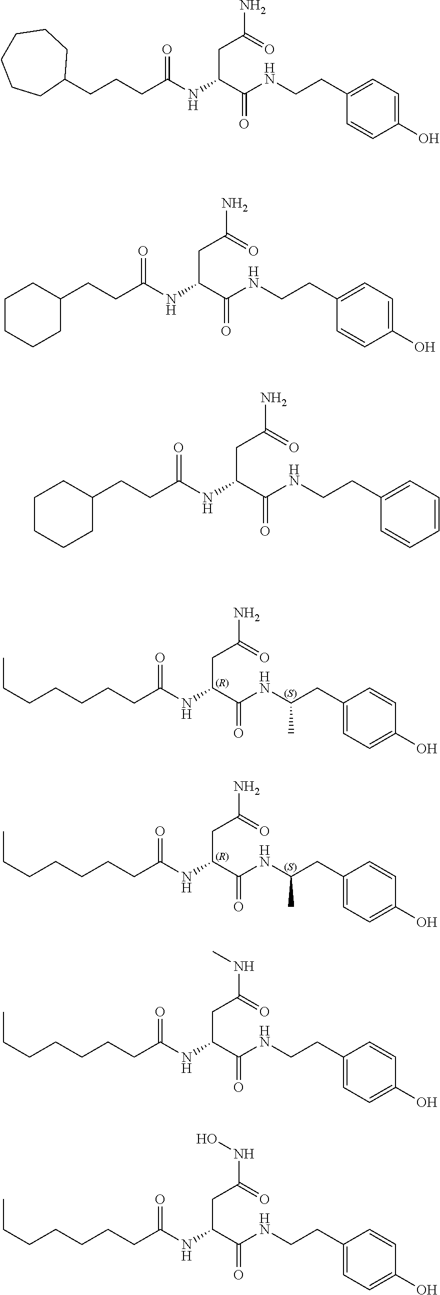 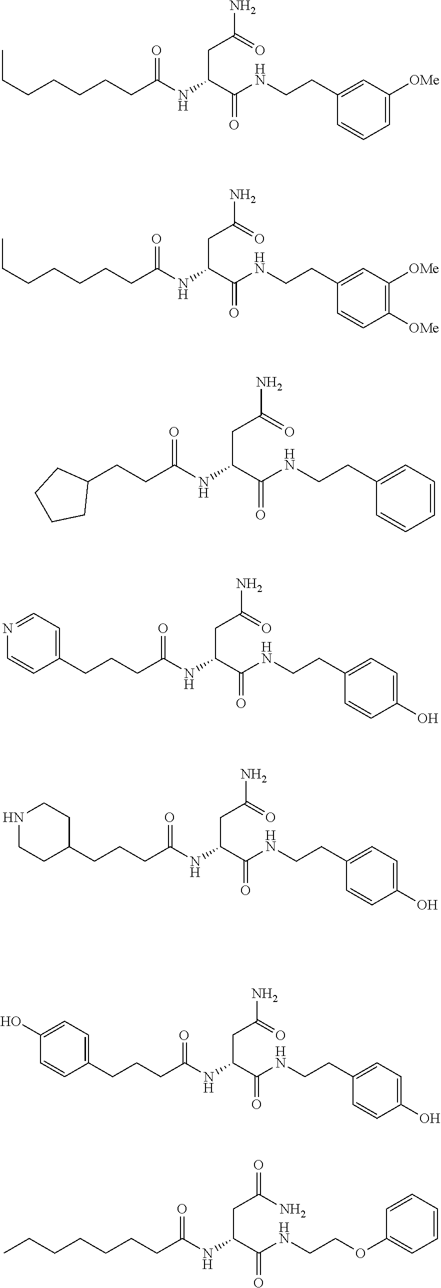 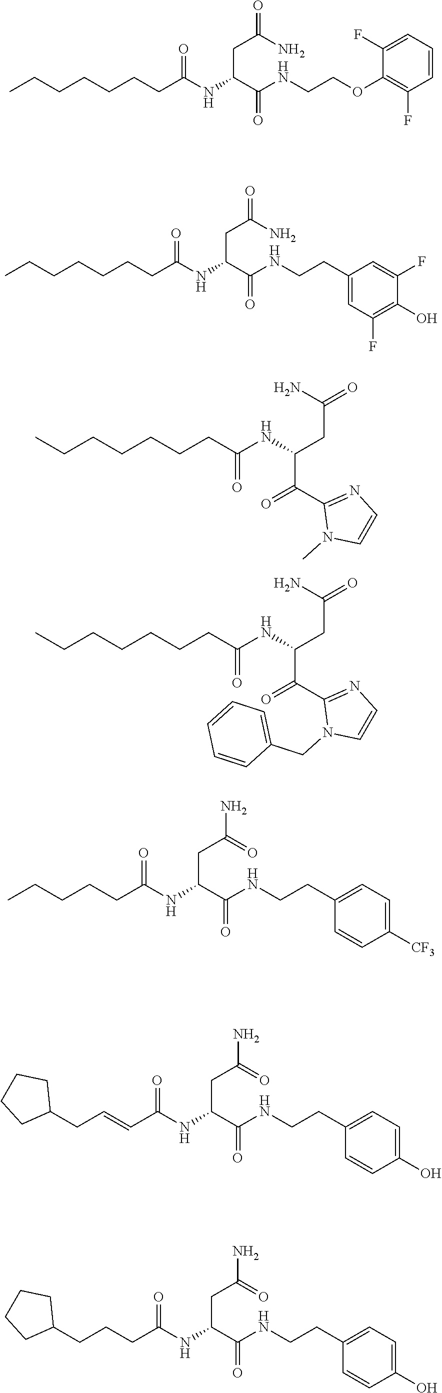 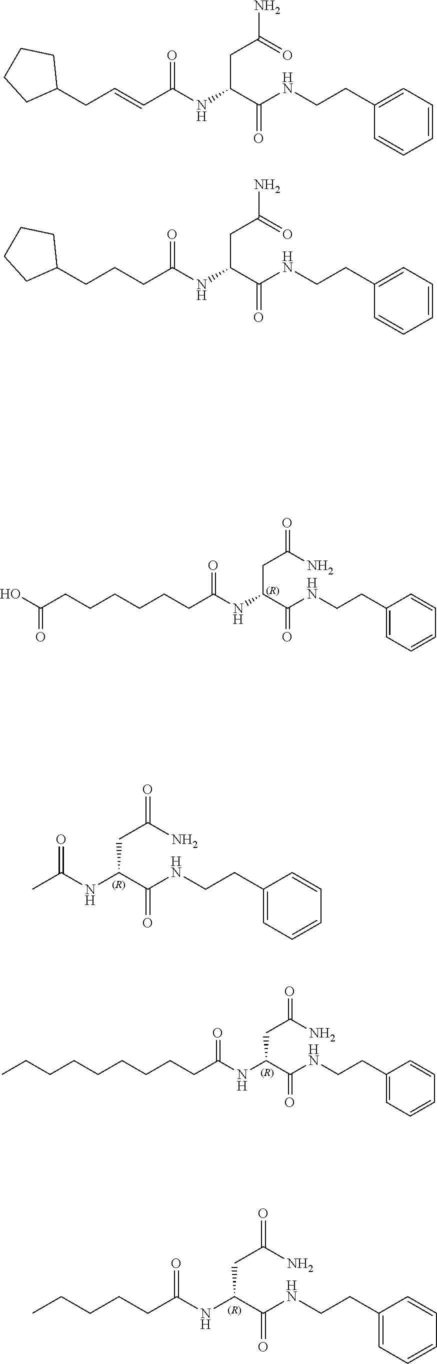 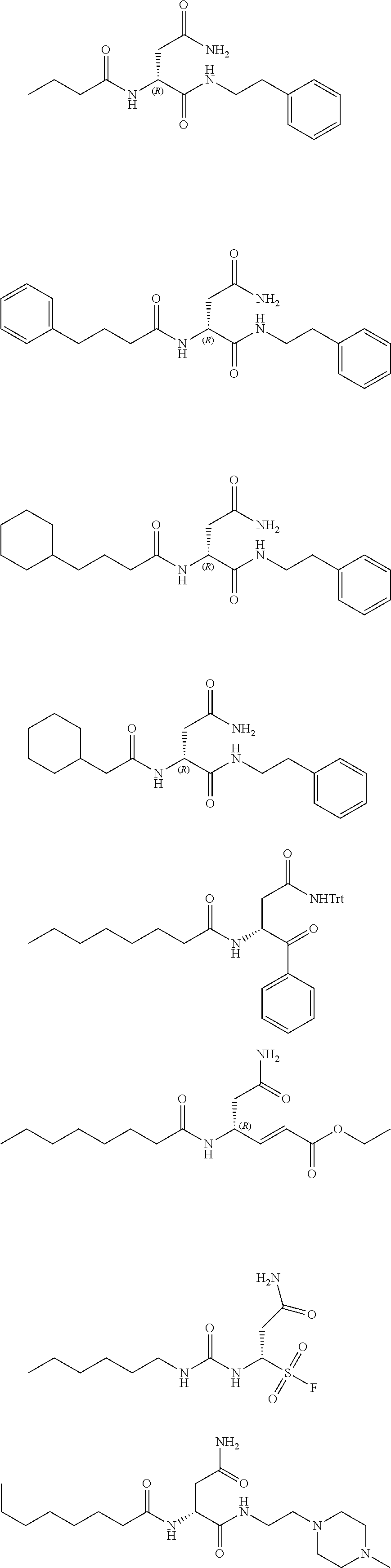 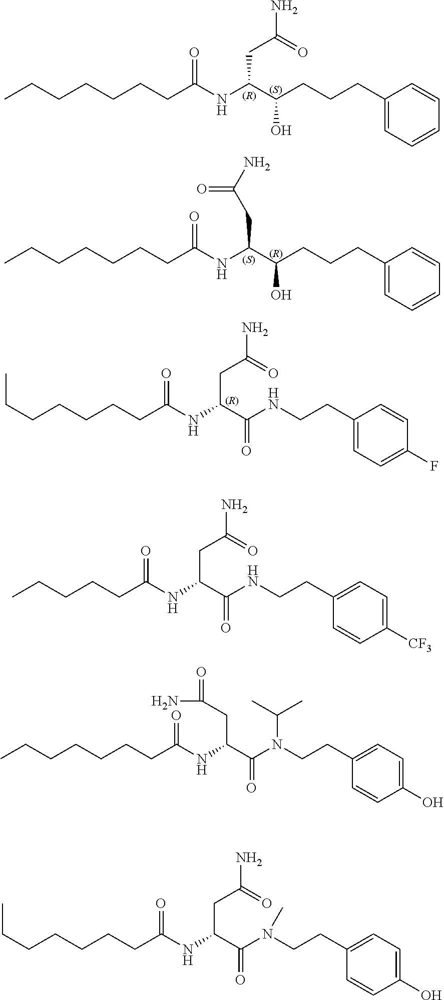 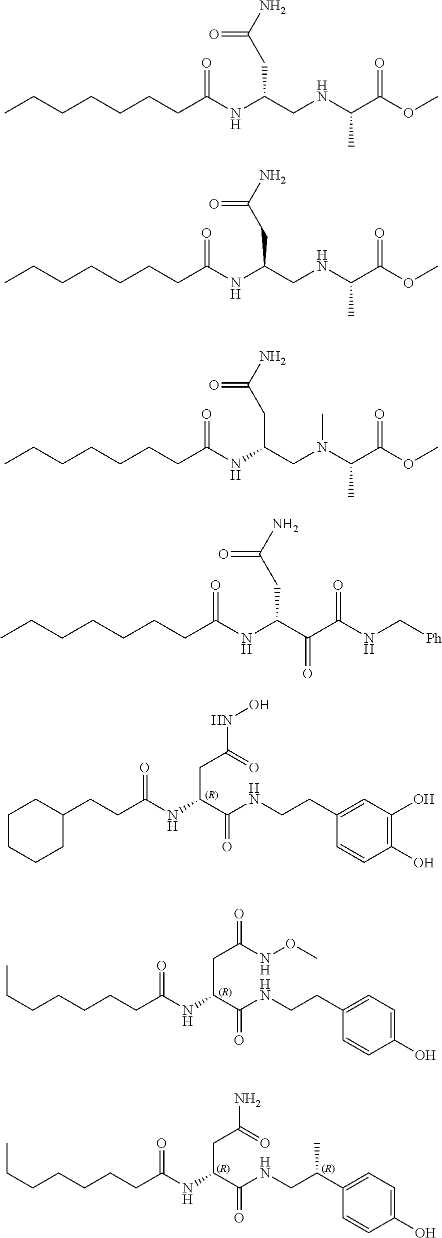 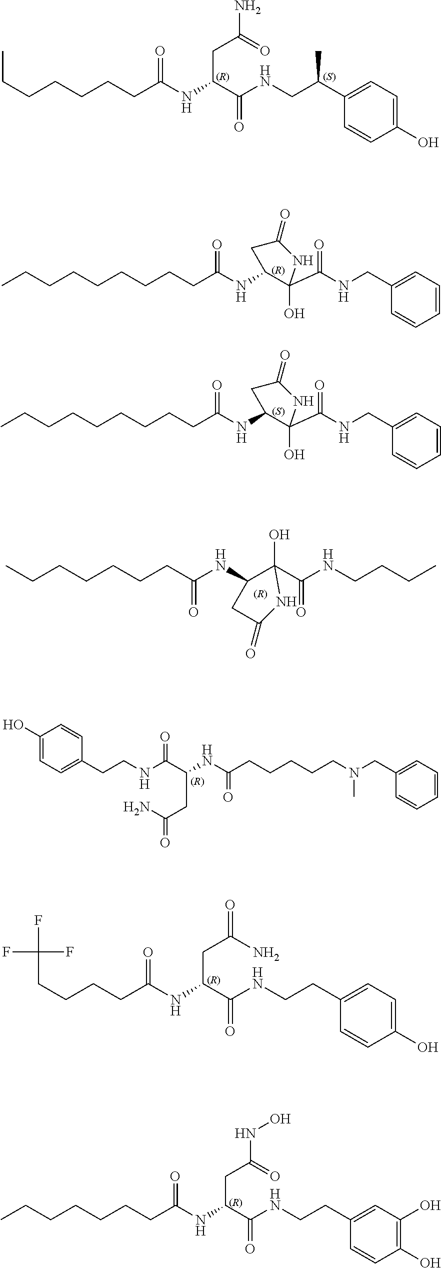 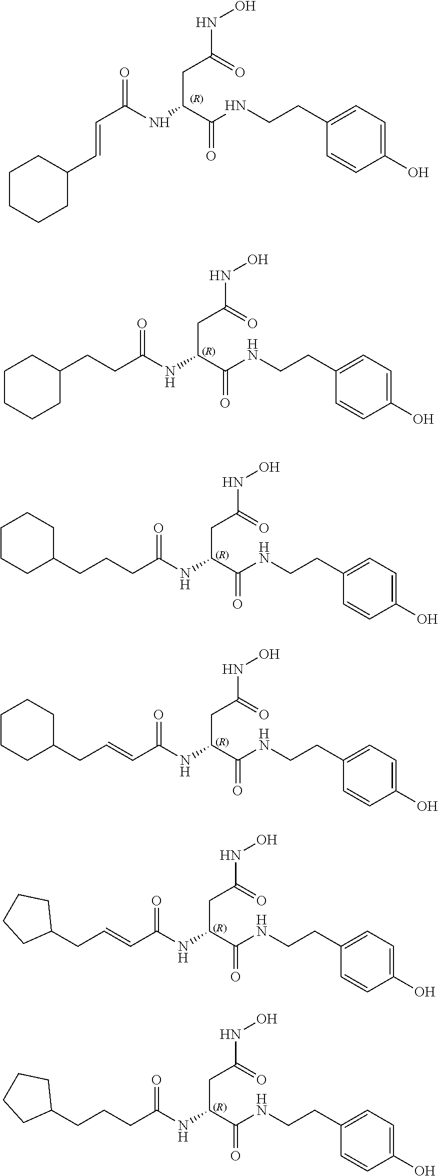 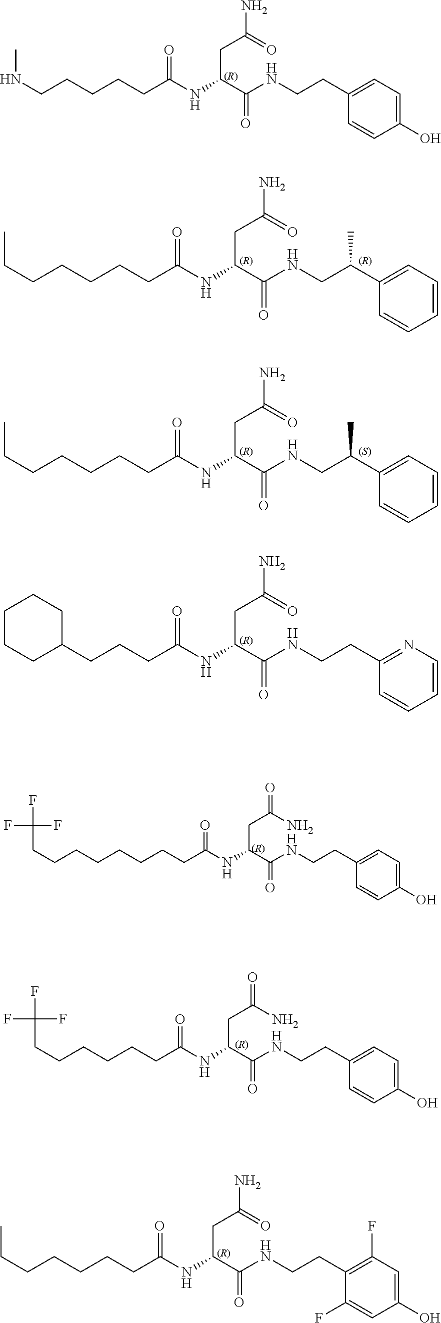 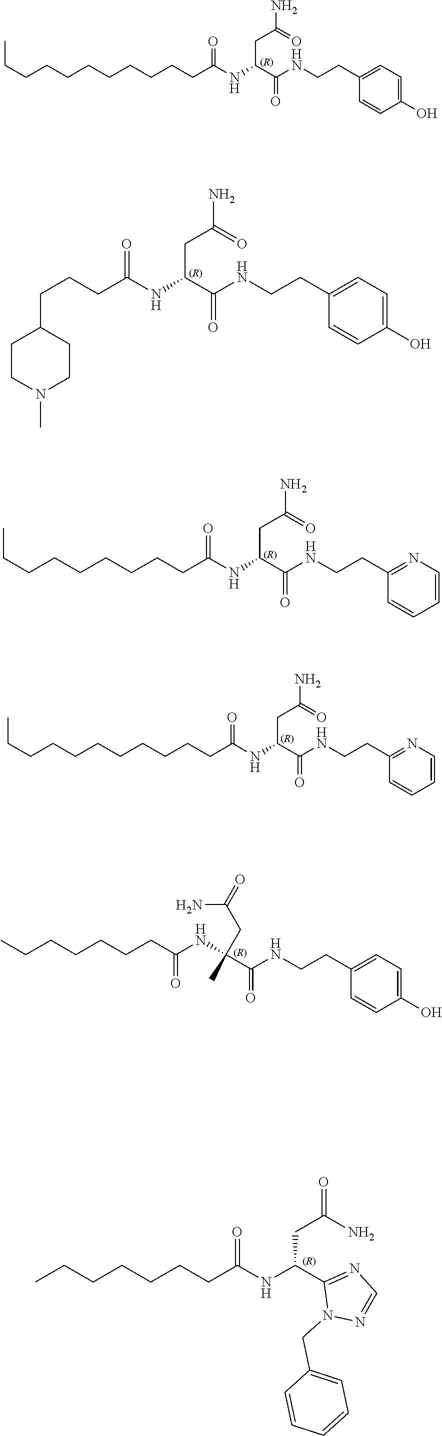
pharmaceutically acceptable salts thereof, and solvates of any of the foregoing.

6. A compound of Formula (I): 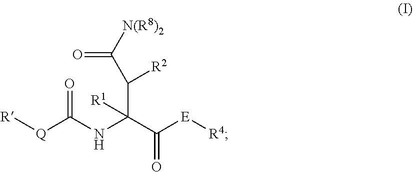
a pharmaceutically acceptable salt thereof, or a solvate of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
E is chosen from CR³R³′, NR³, and O, wherein optionally R¹ and R³ or R² and R³ together form a ring;
R³ and R^{3′} each independently is chosen from H, halogen, and C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁_alkyl-R⁵, C(═O)—C₀₋₆ alkyl-R⁵, O-alkyl or O-aryl is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.

7. The compound of claim 6, wherein R′ is heptyl.

8. The compound of claim 6 or 7, wherein Q is NH.

9. The compound of claim 6 or 7, wherein Q is CH₂.

10. A compound of Formula (II): 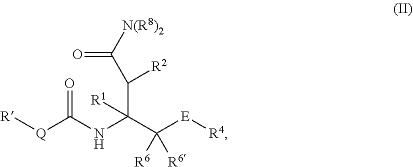
a pharmaceutically acceptable salt thereof, or a solvate of any of the foregoing, wherein:
Q is chosen from CR″R″′, NR″, and 0;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
E is chosen from CR³R³′, NR³, and O, wherein optionally R¹ and R³ or R² and R³ together form a ring;
R³ and R^{3′} each independently is chosen from H, halogen, or C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl, and O-aryl; wherein the C₁₋₇ alkyl, C₁₋₆ alkyl-R⁵, C(═O)—C₀₋₆ alkyl-R⁵, O-alkyl or O-aryl is optionally substituted;
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted; and
R⁶ and R^{6′} each independently is chosen from H, OH, haloalkyl, and C₁₋₃ alkyl, or R⁶ and R^{6′} together form a 4-6 membered ring.

11. The compound of claim 10, wherein R′ is heptyl.

12. The compound of claim 10 or 11, wherein Q is NH.

13. The compound of claim 10 or 11, wherein Q is CH₂.

14. A compound of Formula (III): 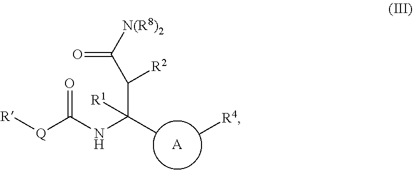
a pharmaceutically acceptable salt thereof, or a solvate of any of the foregoing, wherein:
Q is chosen from CR″R″′, NR″, and O;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
Ring A is chosen from 4-8 membered heteroaryl and 4-8 membered heterocyclyl; wherein the 4-8 membered heteroaryl or 4-8 membered heterocyclyl is optionally substituted;
R⁴ is chosen from C₁₋₇ alkyl, C₁₋₆ alkyl-R⁵, C(═O)—C₀₋₆ alkyl-R⁵, Cl, F, NH₂, O-alkyl, and O-aryl; wherein the C₁₋₇ alkyl, C₁₋₆ alkyl-R⁵, C(═O)—C₀₋₆ alkyl-R⁵, O-alkyl or O-aryl is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.

15. The compound of claim 14, wherein R′ is heptyl.

16. The compound of claim 14 or 15, wherein Q is NH.

17. The compound of claim 14 or 15, wherein Q is CH₂.

18. A compound of Formula (IV): 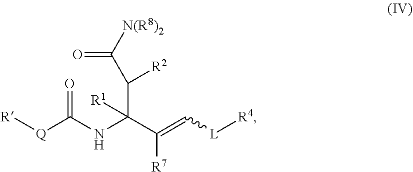
a pharmaceutically acceptable salt thereof, or a solvate of any of the foregoing, wherein:
Q is chosen from CR″R″′, NR″, and 0;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
L is chosen from S(═O)₂, C(═O), C(═O)NR³, and a bond;
R³ and R^{3′} each independently is chosen from H, halogen, and C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted;
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted; and
R⁷ is chosen from H, C₁₋₃ alkyl, and halogen.

19. The compound of claim 18, wherein R′ is heptyl.

20. The compound of claim 18 or 19, wherein Q is NH.

21. The compound of claim 18 or 19, wherein Q is CH₂.

22. A compound of Formula (V): 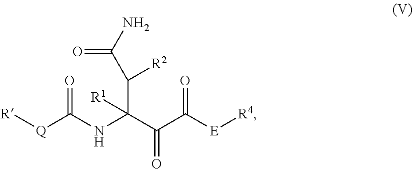
a pharmaceutically acceptable salt thereof, or a solvate of any of the foregoing,
Q is chosen from CR″R″′, NR″, and 0;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
E is chosen from CR³R³′, NR³, and O, wherein optionally R¹ and R³ or R² and R³ together form a ring;
R³ and R^{3′} each independently is chosen from H, halogen, and C₁₋₃ alkyl;
R⁴ is chosen from C₁₋₇ alkyl, C₁₋₆ alkyl-R⁵, C(═O)—C₀₋₆ alkyl-R⁵, Cl, F, NH₂, O-alkyl and O-aryl; wherein the C₁₋₇ alkyl, C₁₋₆ alkyl-R⁵, C(═O)—C₀₋₆ alkyl-R⁵, O-alkyl or O-aryl is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.

23. The compound of claim 22, wherein R′ is heptyl.

24. The compound of claim 22 or 23, wherein Q is NH.

25. The compound of claim 22 or 23, wherein Q is CH₂.

26. A compound of Formula (VI): 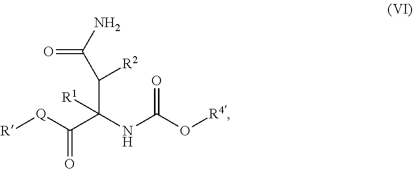
a pharmaceutically acceptable salt thereof, or a solvate of any of the foregoing, pharmaceutically acceptable salts thereof, and solvates of any of the foregoing, wherein:
Q is chosen from CR″R″′, NR″, and 0;
R′ is chosen from hydrogen, C₁₋₁₄ alkyl, and C₁₋₁₄ alkenyl;
R″ and R″′ each independently is chosen from H, C₁₋₃ alkyl, and C₃₋₆ cycloalkyl;
R¹ and R² each independently is chosen from H, halogen, C₁₋₃ alkyl, and C₃₋₆ cycloalkyl;
R⁸ each independently is chosen from H, OH, O-alkyl, O-aryl, and C₁₋₆ alkyl;
R^{4′} is chosen from C₁₋₇ alkyl and C₀₋₆ alkyl-R⁵; wherein the C₁₋₇ alkyl or C₀₋₆ alkyl-R⁵ is optionally substituted; and
R⁵ is chosen from C₆₋₁₀ aryl, 5-10 membered heteroaryl, 3-10 member cycloalkyl, 3-10 membered heterocycloalkyl and C(═O)O—C₁₋₆ alkyl; wherein the C₆₋₁₀ aryl, 5-10 membered heteroaryl, C₃₋₁₀ membered heterocycloalkyl, 3-10 member cycloalkyl, or C(═O)O—C₁₋₆ alkyl are optionally substituted.

27. The compound of claim 26, wherein R′ is heptyl.

28. The compound of claim 26 or 27, wherein Q is NH.

29. The compound of claim 26 or 27, wherein Q is CH₂.

30. A pharmaceutical composition comprising at least one pharmaceutically acceptable excipient and at least one entity chosen from compounds of any one of claims 1 to 29, pharmaceutically acceptable salts thereof, and solvates of any of the foregoing.

31. A method of treating a disease in a subject in need thereof, the method comprising administering to the subject a therapeutically effective amount of the at least one entity chosen from compounds of any one of claims 1 to 29, pharmaceutically acceptable salts thereof, and solvates of any of the foregoing or a pharmaceutical composition of claim 30.

32. A method of treating a disease or condition associated with colibactin expression, the method comprising administering to the subject a therapeutically effective amount of the at least one entity chosen from compounds of any one of claims 1 to 29, pharmaceutically acceptable salts thereof, and solvates of any of the foregoing or a pharmaceutical composition of claim 30.

33. The method of claim 31 or 32, wherein the disease is cancer or familial adenomatous polyposis (FAP).

34. The method of claim 33, wherein the cancer is colorectal cancer.

35. A method of modulating a cancer marker in a subject in need thereof, the method comprising administering to the subject a therapeutically effective amount of the at least one entity chosen from compounds of any one of claims 1 to 29, pharmaceutically acceptable salts thereof, and solvates of any of the foregoing.

36. The method of claim 35, wherein the cancer marker is a colorectal cancer marker selected from carbohydrate antigen 19-9 and/or carcinoembryonic antigen levels in the subject's blood, plasma, serum, stool, or urine.

37. A method of treating colorectal cancer in a patient in need thereof, comprising:
(a) determining from a sample that the patient has a detectable level of a colibactin-adenine adduct or a metabolic derivative thereof, and
(b) administering an inhibitor of colibactin synthesis to the patient.

38. A method of treating a colorectal cancer patient in need thereof, comprising:
(a) obtaining a biological sample from the patient;
(b) determining from the sample that the patient has a detectable level of a colibactin-adenine adduct or a metabolic derivative thereof, and
(c) administering an inhibitor of colibactin synthesis to the patient.

39. The method of claim 38, wherein the biological sample comprises a plasma sample, a urine sample, a stool sample, an intestinal sample, or a combination thereof.

40. The method of any one of claims 37 to 39, wherein the inhibitor of colibactin synthesis is a ClbP inhibitor or an antibiotic.
